(19) United States
(12) Patent Application Publication
(10) Pub. No.: US 2025/0115600 A1
(43) Pub. Date: Apr. 10, 2025

(54) COVALENT MODIFIERS OF AKT1 AND USES THEREOF

(71) Applicants: Terremoto Biosciences, Inc., South San Francisco, CA (US); The Regents of the University of California, Oakland, CA (US)

(72) Inventors: John Taunton, Oakland, CA (US); Gregory B. Craven, Oakland, CA (US); Solomon H. Reisberg, San Mateo, CA (US); Kin S. Yang, San Mateo, CA (US); Chu Hang, San Mateo, CA (US); Jordan D. Carelli, San Francisco, CA (US); Peter A. Thompson, Kirkland, WA (US)

(21) Appl. No.: 18/819,786

(22) Filed: Aug. 29, 2024

Related U.S. Application Data
(63) Continuation of application No. PCT/US2023/063513, filed on Mar. 1, 2023.
(60) Provisional application No. 63/315,933, filed on Mar. 2, 2022.

(51) Int. Cl. C07D 471/04; A61K 31/437; A61P 35/00; C12N 9/12

(52) U.S. Cl. CPC C07D 471/04; A61K 31/437; A61P 35/00; C12N 9/12; C12Y 207/11001

(57) ABSTRACT

Described herein are compounds and methods for the modulation of AKT1 proteins, such as AKT1 E17K. In some aspects, the present disclosure provides an AKT1 protein, wherein a compound is bound to a lysine residue of the AKT1 protein. In another aspect, the present disclosure provides compounds of Formula (I) and (II), useful for the modulation of AKT1 proteins. In another aspect, the instant disclosure provides a method of attenuating AKT1 activity using the compounds described herein.

### CROSS REFERENCE TO RELATED APPLICATIONS

This application is the by-pass continuation of International Application No. PCT/US2023/063513, filed Mar. 1, 2023, which claims the benefit of U.S. Provisional Application No. 63/315,933, filed Mar. 2, 2022, the entire contents of each of which are incorporated herein by reference.

### SEQUENCE LISTING

The instant application contains a Sequence Listing which has been submitted electronically in XML file format and is hereby incorporated by reference in its entirety. Said XML copy, created on May 15, 2023, is named 61402-704_601_SL.xml and is 12,141 bytes in size.

### BACKGROUND OF THE INVENTION

The AKT or Protein Kinase B (PKB) family of serine/threonine protein kinases is comprised of 3 highly homologous members, AKT1, AKT2 and AKT3. The family of AKT proteins are involved in signal transduction pathways that regulate cellular processes including apoptosis, proliferation, differentiation and metabolism. The AKT1 pathway is the most frequently deregulated signaling pathways in human cancers. Enhanced activation of all the isoforms can be implicated in tumor development and progression, and has been demonstrated in breast, ovarian, pancreatic, and prostate cancers among others (Song et al., 2019). In cancer cells, AKT1 is involved in proliferation and growth, promoting tumor initiation and suppressing apoptosis, whereas AKT2 regulates cytoskeleton dynamics, favoring local tissue invasion and metastasis. The role of AKT3 hyperactivation in cancer is hypothesized to be involved with possible stimulation of cell proliferation (Hinz, N. et al. Cell Commun Signal 2019, 17(1), 154; Pascual, J. et al. Ann. Oncol. 2019, 30(7), 1051-1060). Expression of these AKT family members is altered in many human malignant carcinomas including gastric, breast, prostate, ovarian and pancreatic. AKT family members are rarely mutated however, the most common mutation is AKT1 E17K which has been reported in 6-8% of breast cancers, 2-6% of colorectal cancers, and in 6% of meningiomas, in human (Yu, Y., et al. PLoS One 2015, 10 (10) No. e0140479). Thus, there is a need to develop new treatments for the modulation of AKT1 and mutants thereof.

### SUMMARY OF THE INVENTION

In one aspect, the present disclosure provides an AKT1 protein covalently bound to a compound, wherein the compound is covalently bound to a lysine residue of the AKT1 protein. In some embodiments, the compound is an exogenous AKT1 modulator. In some embodiments, the compound is an exogenous AKT1 inhibitor. In some embodiments, the AKT1 protein comprises a E17K mutation, a E40K mutation, or a E49K mutation. In some embodiments, the lysine residue is selected from K17, K40, K49, K158, K163, K179, K267, and K297. In some embodiments, the lysine residue is K17. In some embodiments, the lysine residue is selected from K40, K49, K158, K163, K179, K267, and K297. In some embodiments, the AKT1 protein is in vivo. In some embodiments, the AKT1 protein is an in vivo engineered AKT1 protein, wherein the in vivo engineered AKT1 protein is generated by contacting the AKT1 protein in vivo with the compound. In some embodiments, the in vivo engineered AKT1 protein is a human in vivo engineered AKT1 protein. In some embodiments, the covalent bond between the compound and the lysine residue is a reversible covalent bond. In some embodiments, the reversible covalent bond in the in vivo AKT1 protein is a carbon-nitrogen double bond. In some embodiments, the carbon-nitrogen double bond results from a reversible reaction between the amine functional group of K17 and an aldehyde functional group on the compound. In some embodiments, the aldehyde functional group is an aromatic aldehyde.

In another aspect, the present disclosure provides an in vivo engineered AKT1 protein comprising a non-naturally occurring reversible covalent modification at a lysine residue, the reversible covalent modification being generated from an in vivo nucleophilic reaction between an exogenous aromatic aldehyde and a lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the AKT1 protein comprises a E17K mutation. In some embodiments, the lysine residue is selected from K17, K40, K49, K158, K163, K179, K267, and K297. In some embodiments, the lysine residue is K17. In some embodiments, the lysine is selected from K40, K49, K158, K163, K179, K267, and K297. In some embodiments, the in vivo engineered AKT1 protein is a human in vivo engineered AKT1 protein.

In another aspect, the present disclosure provides a method of covalently modifying an AKT1 protein, comprising contacting the AKT1 protein with an exogenous AKT1 modulator, wherein the AKT1 modulator comprises a reversible electrophilic moiety thereby forming a reversible covalent AKT1 adduct. In some embodiments, the contacting is in vitro. In some embodiments, the contacting is in vivo. In some embodiments, the AKT1 modulator is an AKT1 inhibitor. In some embodiments, the reversible electrophilic moiety is an aromatic aldehyde.

In another aspect, the present disclosure provides a method of attenuating AKT1 activity, comprising contacting AKT1 protein with an AKT1 inhibitor, wherein the AKT1 inhibitor comprises a reversible electrophilic moiety. In some embodiments, the contacting is in vitro. In some embodiments, following the contacting, the AKT1 activity is attenuated by 50% or more relative to a control in the absence of the exogenous AKT1 inhibitor. In some embodiments, following the contacting, the AKT1 activity is attenuated by 70% or more relative to a control in the absence of the exogenous AKT1 inhibitor.

In another aspect, the present disclosure provides a compound of Formula (I):

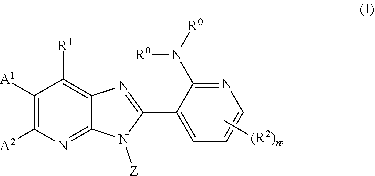

or a pharmaceutically acceptable salt thereof; wherein,
   R⁰ is independently selected at each occurrence from hydrogen, alkyl, heteroalkyl, and alkenyl, alkynyl, any of which is independently unsubstituted or substituted;
   R¹ and R² are each independently selected from hydrogen, halogen, alkyl, heteroalkyl, alkenyl, alkynyl, —OR¹⁰, —SR¹⁰, —N(R¹⁰)₂, —NO₂, and —CN, wherein each alkyl, heteroalkyl, alkenyl, and alkynyl of R¹ and R² is independently unsubstituted or substituted;
   n is selected from 0, 1, 2, and 3;
   A¹ and A² are each independently selected from: hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, —CN, alkyl, heteroalkyl, alkenyl, alkynyl, carbocycle and heterocycle, wherein each alkyl, heteroalkyl, alkenyl, alkynyl, carbocycle and heterocycle of A¹ and A² is independently unsubstituted or substituted;
   Z is represented by

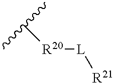

wherein,
   R²⁰ is selected from heterocyclene and phenylene, any of which is unsubstituted or substituted;
   L is a bond or represented by -L¹-L²-L³-L⁴-, wherein each L¹, L², L³, and L⁴ is independently selected from (a) and (b):
      (a) —O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, —(R¹⁴)NC(O)N(R¹⁴)—, and —(R¹⁴)NC(O)N(R¹⁴)N(R¹⁴)—; and
      (b) alkylene, heteroalkylene, alkenylene, alkynylene, carbocyclene, and heterocyclene, any of which is independently unsubstituted or substituted;
      wherein L², L³, and L⁴ are each optionally absent;
      wherein no more than two of L¹, L², L³, and L⁴ are selected from (a) and the two selected are not adjacent to each other;
   R²¹ is selected from heterocycle and carbocycle, any of which is substituted with C(O)H, wherein R²¹ is optionally further substituted; and
R¹⁰, R¹¹, and R¹⁴ are each independently selected at each occurrence from hydrogen, alkyl, heteroalkyl, carbocycle, and heterocycle, any of which is independently unsubstituted or substituted.

In some embodiments, the compound or salt of Formula (I) is a compound represented by the structure of Formula (I-A):

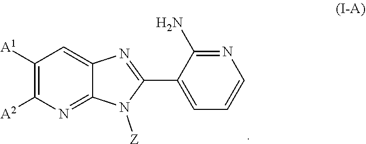

or a pharmaceutically acceptable salt thereof.

In another aspect, the present disclosure provides a compound of Formula (II):

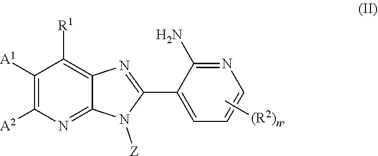

or a pharmaceutically acceptable salt thereof; wherein,
   R¹ and R² are each independently selected from hydrogen, halogen, C₁₋₄ alkyl, C₁₋₄ haloalkyl, —OR¹⁰, —SR¹⁰, —N(R¹⁰)₂, —NO₂, and —CN;
   n is selected from 0, 1, 2, and 3;
   A¹ and A² are each independently selected from:
      hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, and —CN;
      C₁₋₆ alkyl, C₂₋₆ alkyenyl, and C₂₋₆ alkynyl, any of which is optionally substituted with one or more substituents independently selected from halogen, —OR¹¹, —SR¹¹, —N(R¹¹)₂, —C(O)N(R¹¹)₂, —N(R¹¹)C(O)R¹¹, ═O, ═S, ═N(R¹¹), and —CN; and
      3- to 10-membered heterocycle and C₃₋₁₀ carbocycle, any one of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
         C₁₋₆ alkyl, C₂₋₆ alkenyl, and C₃₋₆ alkynyl, any one of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), and —CN; and
         C₃₋₁₀ carbocycle and 3- to 10-membered heterocycle; any one of which is optionally substituted with one or more substituents selected from halogen, C₁₋₄ alkyl, C₁₋₄ haloalkyl, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O;
   Z is represented by

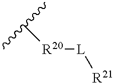

wherein,
   R²⁰ is selected from 5- to 6-membered heterocyclene and phenylene, any of which is optionally substituted with one or more substituents independently selected from halogen, C₁₋₄ alkyl, C₁₋₄ haloalkyl —OR¹², —SR¹², —N(R¹²)₂, —NO₂, ═O, ═S, ═N(R¹²), and —CN;
   L is a bond or represented by -L¹-L²-L³-L⁴-, wherein each L¹, L², L³, and L⁴ is independently selected from (a) and (b):
      (a) —O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, (R¹⁴)NC(O)N(R¹⁴), and (R¹⁴)NC(O)N(R¹⁴)N(R¹⁴); and
      (b) C₁₋₆ alkylene, C₂₋₆ alkenylene, C₂₋₆ alkynylene, C₃₋₈ carbocyclene, and 3- to 8-membered heterocyclene, any one of which is optionally substituted with one or more substituents independently selected from halogen, —OR¹³, —SR¹³, —N(R¹³)₂, ═O, ═S, and —CN;
      wherein L², L³, and L⁴ are each optionally absent;
      wherein no more than two of L¹, L², L³, and L⁴ are selected from (a) and the two selected are not adjacent to each other;
   R²¹ is selected from 5- to 6-membered heterocycle and C₃₋₆ carbocycle, any of which is substituted with —C(O)H, wherein R²¹ is further optionally substituted with one or more substituents independently selected from:
      halogen, C₁₋₄ alkyl, C₁₋₄ haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN; and
R¹⁰, R¹¹, R¹², R¹³, R¹⁴, and R¹⁵ are each independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O.

In some embodiments, the compound or salt of Formula (II) is a compound represented by the structure of Formula (II-A):

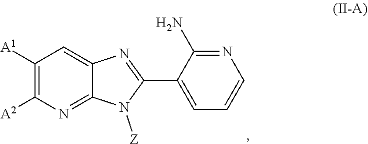

or a pharmaceutically acceptable salt thereof.

In some embodiments, the compound or salt of Formula (II) is a compound represented by the structure of Formula (II-B):

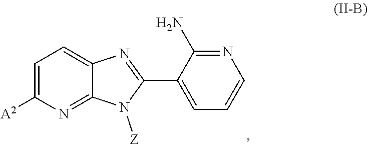

or a pharmaceutically acceptable salt thereof.

In some embodiments, the compound or salt of Formula (II) is a compound represented by the structure of Formula (II-C):

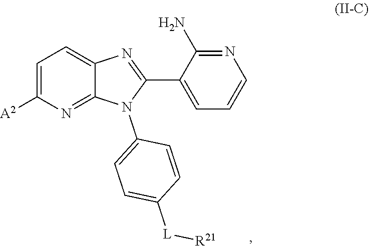

or pharmaceutically acceptable salt thereof.

In some embodiments, the compound is a compound of Table 1, or a salt of any one thereof.

In another aspect, the present disclosure provides a pharmaceutical composition comprising a pharmaceutically acceptable excipient and a compound of Formula (I) or (II), or a pharmaceutically acceptable salt thereof.

In another aspect, the present disclosure provides a method of modulating activity of mutant AKT1 comprising, administering to a subject in need thereof a compound of Formula (I) or (II), or a pharmaceutically acceptable salt thereof, or a pharmaceutical composition of a compound of Formula (I) or (II), or a pharmaceutically acceptable salt thereof.

In another aspect, the present disclosure provides a method of selectively modulating activity a mutant AKT1 over a wild type AKT comprising administering to a subject in need thereof a compound of Formula (I) or (II), or a pharmaceutically acceptable salt thereof, or a pharmaceutical composition of a compound of Formula (I) or (II), or a pharmaceutically acceptable salt thereof, wherein the wild type AKT is selected from wild type AKT1 and wild type AKT2.

In another aspect, the present disclosure provides a method of treating cancer in a subject in need thereof, the method comprising administering to the subject a compound of Formula (I) or (II), or a pharmaceutically acceptable salt thereof, or a pharmaceutical composition of a compound of Formula (I) or (II), or a pharmaceutically acceptable salt thereof. In some embodiments, the cancer is selected from breast cancer, colorectal cancer, and meningioma. In some embodiments, the administration modulates activity of a mutant AKT1. In some embodiments, the mutant AKT1 is AKT1 E17K.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-B provides intact-protein mass spectra for the AKT1 WT protein incubated with Compound 133, both without a competitor and with a significant stoichiometric excess of a competitor molecule ARQ-092, respectively. FIG. 1C-D provides intact-protein mass spectra for the AKT1 E17K mutant protein incubated with Compound 133, both without and with a significant stoichiometric excess of a competitor molecule ARQ-092, respectively.

FIG. 2A-B provides intact-protein mass spectra for the AKT1 WT protein incubated with Compound 132, both without a competitor and with a significant stoichiometric excess of a competitor molecule ARQ-092, respectively. FIG. 2C-D provides intact-protein mass spectra for the AKT1 E17K mutant protein incubated with Compound 132, both without a competitor and with a significant stoichiometric excess of a competitor molecule ARQ-092, respectively.

FIG. 3A-B provides intact-protein mass spectra for the AKT1 WT protein incubated with Compound 125, both without a competitor and with a significant stoichiometric excess of a competitor molecule ARQ-092, respectively. FIG. 3C-D provides intact-protein mass spectra for the AKT1 E17K mutant protein incubated with Compound 125 both without a competitor and with a significant stoichiometric excess of a competitor molecule ARQ-092, respectively.

FIG. 4A-B provides intact-protein mass spectra for the AKT1 WT protein incubated with Compound 122, both without a competitor and with a significant stoichiometric excess of a competitor molecule ARQ-092, respectively. FIG. 4C-D provides intact-protein mass spectra for the AKT1 E17K mutant protein incubated with Compound 122 both without a competitor and with a significant stoichiometric excess of a competitor molecule ARQ-092, respectively.

FIG. 5A-B provides intact-protein mass spectra for the AKT1 WT protein incubated with Compound 114 both without a competitor and with a significant stoichiometric excess of a competitor molecule ARQ-092, respectively. FIG. 5C-D provides intact-protein mass spectra for the AKT1 E17K mutant protein incubated with Compound 114 both without a competitor and with a significant stoichiometric excess of a competitor molecule ARQ-092, respectively.

FIG. 6A-B provides intact-protein mass spectra for the AKT1 WT protein incubated with Compound 110, both without a competitor and with a significant stoichiometric excess of a competitor molecule ARQ-092, respectively. FIG. 6C-D provides intact-protein mass spectra for the AKT1 E17K mutant protein incubated with Compound 110, both without a competitor and with a significant stoichiometric excess of a competitor molecule ARQ-092, respectively.

FIG. 7A-B provides intact-protein mass spectra for the AKT1 WT protein incubated with Compound 107, both without a competitor and with a significant stoichiometric excess of a competitor molecule ARQ-092, respectively. FIG. 7C-D provides intact-protein mass spectra for the AKT1 E17K mutant protein incubated with Compound 107, both without a competitor and with a significant stoichiometric excess of a competitor molecule ARQ-092, respectively.

FIG. 8A-B provides intact-protein mass spectra for the AKT1 WT protein incubated with Compound 96, both without a competitor and with a significant stoichiometric excess of a competitor molecule ARQ-092, respectively. FIG. 8C-D provides intact-protein mass spectra for the AKT1 E17K mutant protein incubated with Compound 96 both without a competitor and with a significant stoichiometric excess of a competitor molecule ARQ-092, respectively.

FIG. 9A-B provides intact-protein mass spectra for the AKT1 WT protein incubated with Compound 94 both without a competitor and with a significant stoichiometric excess of a competitor molecule ARQ-092, respectively. FIG. 9C-D provides intact-protein mass spectra for the AKT1 E17K mutant protein incubated with Compound 94, both without a competitor and with a significant stoichiometric excess of a competitor molecule ARQ-092, respectively.

FIG. 10A-B provides intact-protein mass spectra for the AKT1 WT protein incubated with Compound 91, both without a competitor and with a significant stoichiometric excess of a competitor molecule ARQ-092, respectively. FIG. 10C-D provides intact-protein mass spectra for the AKT1 E17K mutant protein incubated with Compound 91, both without a competitor and with a significant stoichiometric excess of a competitor molecule ARQ-092, respectively.

FIG. 11A-B provides intact-protein mass spectra for the AKT1 WT and E17K mutant protein incubated with Compound 75, respectively.

FIG. 12A-B provides intact-protein mass spectra for the AKT1 WT protein incubated with Compound 74, both without a competitor and with a significant stoichiometric excess of a competitor molecule ARQ-092, respectively. FIG. 12C-D provides intact-protein mass spectra for the AKT1 E17K mutant protein incubated with Compound 74 both without a competitor and with a significant stoichiometric excess of a competitor molecule ARQ-092, respectively.

FIG. 13A-B provides intact-protein mass spectra for the AKT1 WT and E17K mutant protein incubated with Compound 73, respectively.

FIG. 14A-B provides intact-protein mass spectra for the AKT1 WT protein incubated with Compound 72, both without a competitor and with a significant stoichiometric excess of a competitor molecule ARQ-092, respectively. FIG. 14C-D provides intact-protein mass spectra for the AKT1 E17K mutant protein incubated with Compound 72, both without a competitor and with a significant stoichiometric excess of a competitor molecule ARQ-092, respectively.

FIG. 15A-B provides intact-protein mass spectra for the AKT1 WT protein incubated with Compound 58, both without a competitor and with a significant stoichiometric excess of a competitor molecule ARQ-092, respectively. FIG. 15C-D provides intact-protein mass spectra for the AKT1 E17K mutant protein incubated with Compound 58, both without a competitor and with a significant stoichiometric excess of a competitor molecule ARQ-092, respectively.

FIG. 16A-B provides intact-protein mass spectra for the AKT1 WT protein incubated with Compound 56, both without a competitor and with a significant stoichiometric excess of a competitor molecule ARQ-092, respectively. FIG. 16C-D provides intact-protein mass spectra for the AKT1 E17K mutant protein incubated with Compound 56, both without a competitor and with a significant stoichiometric excess of a competitor molecule ARQ-092, respectively.

FIG. 17A-B provides intact-protein mass spectra for the AKT1 WT protein incubated with Compound 53, both without a competitor and with a significant stoichiometric excess of a competitor molecule ARQ-092, respectively. FIG. 17C-D provides intact-protein mass spectra for the AKT1 E17K mutant protein incubated with Compound 53, both without a competitor and with a significant stoichiometric excess of a competitor molecule ARQ-092, respectively.

FIG. 18A-B provides intact-protein mass spectra for the AKT1 WT protein incubated with Compound 52, both without a competitor and with a significant stoichiometric excess of a competitor molecule ARQ-092, respectively. FIG. 18C-D provides intact-protein mass spectra for the AKT1 E17K mutant protein incubated with Compound 52, both without a competitor and with a significant stoichiometric excess of a competitor molecule ARQ-092, respectively.

FIG. 19A-B provides intact-protein mass spectra for the AKT1 WT protein incubated with Compound 49, both without a competitor and with a significant stoichiometric excess of a competitor molecule ARQ-092, respectively. FIG. 19C-D provides intact-protein mass spectra for the AKT1 E17K mutant protein incubated with Compound 49, both without a competitor and with a significant stoichiometric excess of a competitor molecule ARQ-092, respectively.

FIG. 20A-B provides intact-protein mass spectra for the AKT1 WT protein incubated with Compound 31, both without a competitor and with a significant stoichiometric excess of a competitor molecule ARQ-092, respectively. FIG. 20C-D provides intact-protein mass spectra for the AKT1 E17K mutant protein incubated with Compound 31, both without a competitor and with a significant stoichiometric excess of a competitor molecule ARQ-092, respectively.

FIG. 21A-B provides intact-protein mass spectra for the AKT1 WT protein incubated with Compound 21, both without a competitor and with a significant stoichiometric excess of a competitor molecule ARQ-092, respectively. FIG. 21C-D provides intact-protein mass spectra for the AKT1 E17K mutant protein incubated with Compound 21, both without a competitor and with a significant stoichiometric excess of a competitor molecule ARQ-092, respectively.

FIG. 22A-B provides intact-protein mass spectra for the AKT1 WT protein incubated with Compound 20, both without a competitor and with a significant stoichiometric excess of a competitor molecule ARQ-092, respectively. FIG. 22C-D provides intact-protein mass spectra for the AKT1 E17K mutant protein incubated with Compound 20, both without a competitor and with a significant stoichiometric excess of a competitor molecule ARQ-092, respectively.

FIG. 23A-B provides intact-protein mass spectra for the AKT1 WT protein incubated with Compound 139, both without a competitor and with a significant stoichiometric excess of a competitor molecule ARQ-092, respectively. FIG. 23C-D provides intact-protein mass spectra for the AKT1 E17K mutant protein incubated with Compound 139, both without a competitor and with a significant stoichiometric excess of a competitor molecule ARQ-092, respectively.

FIG. 24A-B provides intact-protein mass spectra for the AKT1 WT protein incubated with Compound 140, both without a competitor and with a significant stoichiometric excess of a competitor molecule ARQ-092, respectively. FIG. 24C-D provides intact-protein mass spectra for the AKT1 E17K mutant protein incubated with Compound 140, both without a competitor and with a significant stoichiometric excess of a competitor molecule ARQ-092, respectively.

FIG. 25A-B provides intact-protein mass spectra for the AKT1 WT protein incubated with Compound 141, both without a competitor and with a significant stoichiometric excess of a competitor molecule ARQ-092, respectively. FIG. 25C-D provides intact-protein mass spectra for the AKT1 E17K mutant protein incubated with Compound 141, both without a competitor and with a significant stoichiometric excess of a competitor molecule ARQ-092, respectively.

FIG. 26A-B provide 2-D diagram close-ups from the crystal structure of AKT1-E17K/Compound 133 detailing the interactions between Compound 133 and residues of the AKT1 E17K mutant protein.

FIG. 27A-B provide 2-D diagram close-ups from the crystal structure of AKT1-WT/Compound 133-NB41 detailing the interactions between Compound 133 and residues of the AKT1 WT protein. The AKT1 WT protein construct used in FIG. 27A-B corresponds to a truncated AKT1 WT protein comprising an N-terminus truncation of seven amino acid residues. Therefore, for example, residues K290, T204, and Y265 in FIG. 27A-B correspond to residues K297, T211, and Y272 in wild-type human AKT1. Accordingly, the imine bond at the lysine residue labeled K290 in FIG. 27A-B corresponds to an imine bond at K297 in wild-type human AKT1.

FIG. 28A-B provide 2-D diagram close-ups from the crystal structure of AKT1-E17K/Compound 110-NB41 detailing the interactions between Compound 110 and residues of the AKT1 E17K mutant protein.

FIG. 29A-29F illustrates intact-protein mass spectra for different proteins and protein adducts. FIG. 29A-29B illustrate mass spectra for AKT1 wild-type and AKT1 E17K, respectively. FIG. 29C illustrates mass spectra for AKT1 wild-type and Compound A. FIG. 29D illustrates mass spectra for AKT1 E17K and Compound A. FIG. 29E illustrates mass spectra for AKT1 wild-type and Compound B. FIG. 29F illustrates mass spectra for AKT1 E17K and Compound B.

FIG. 30A-30B illustrate MS/MS spectra of AKT1(E17K) and Compound A. The labelled protein was digested with trypsin and the resulting peptides were analyzed on a tandem mass spectrometer. Peptides bearing lysine residues modified with Compound A were identified by spectral matching, with matched fragment ions indicated. FIG. 30A illustrates the covalent modification of AKT1 E17K by Compound A at lysine residue K17. FIG. 30B illustrates the covalent modification of AKT1 E17K by Compound A at lysine residue K297.

FIG. 31 illustrates in-gel fluorescence and western blot wash out experiments of BEAS2B-AKT1(WT) and BEAS2B-AKT1(E17K) following incubation of the cells with Compound B for 0.5 hr. Wash out periods were 0 hr, 0.5 hr, 1 hr, 1.5 hr, 3 hrs, and 21 hrs.

FIG. 32A-32B illustrates off-rate studies of Compounds from wild-type AKT1 and AKT1 E17K. FIG. 32A provides dissociation rate data from wild-type AKT1 and AKT1 E17K treated with Compound A (GTC100) via competition with excess ARQ-092. FIG. 32B provides dissociation rate data from wild-type AKT1 and AKT1 E17K treated with Compound B (GCT137) via competition with excess ARQ-092 (miransertib).

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Defintions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an”, and “the” includes plural references unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below. As used herein, the terms “treatment,” “treat,” and “treating” are meant to include the full spectrum of intervention for the cancer from which the subject is suffering, such as administration of the combination to alleviate, slow, stop, or reverse one or more symptoms of the cancer and to delay the progression of the cancer even if the cancer is not actually eliminated. Treatment can include, for example, a decrease in the severity of a symptom, the number of symptoms, or frequency of relapse, e.g., the inhibition of tumor growth, the arrest of tumor growth, or the regression of already existing tumors.

The term “exogenous” refers to any material introduced from or originating from outside a cell, a tissue or an organism that is not produced by or does not originate from the same cell, tissue, or organism in which it is being introduced. By way of example a chemical compound, nucleic acid or antibody.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

The term “N-terminally positioned” when referring to a position of a first domain or sequence relative to a second domain or sequence in a polypeptide primary amino acid sequence means that the first domain is located closer to the N-terminus of the polypeptide primary amino acid sequence. In some embodiments, there may be additional sequences and/or domains between the first domain or sequence and the second domain or sequence.

The term “C-terminally positioned” when referring to a position of a first domain or sequence relative to a second domain or sequence in a polypeptide primary amino acid sequence means that the first domain is located closer to the C-terminus of the polypeptide primary amino acid sequence. In some embodiments, there may be additional sequences and/or domains between the first domain or sequence and the second domain or sequence.

The term “antibody” is used herein in the broadest sense and encompasses monoclonal antibodies, polyclonal antibodies, dimers, multimers, multispecific antibodies (e.g., bispecific antibodies), and antibody fragments, so long as they exhibit the desired biological activity (Miller et al., J Immunol. 2003, 170, 4854-4861). Antibodies may be murine, human, humanized, chimeric, or derived from other species. (Janeway, C., Travers, P., Walport, M., Shlomchik (2001) Immunol. Biology, 5th Ed., Garland Publishing, New York). Antibodies bound to various types of molecules, such as polyethylene glycols (PEGs), may be used as modified antibodies. Methods for modifying antibodies are already established in the art.

The AKT1 protein described herein includes the naturally occurring (e.g., homo sapien) protein and variants thereof (e.g., Wild-type (WT) AKT1 protein, and mutant AKT1 variants, such as but not limited to E17K AKT1 mutant protein). In some embodiments, the AKT1 protein as described herein can be found in the NCBI database as GenBank: AAL55732.1 (https://www.ncbi.nlm.nih.gov/protein/AAL55732.1).

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

The terms “polypeptide,” “peptide” and “protein” are often used interchangeably herein in reference to a polymer of amino acid residues. A protein, generally, refers to a full-length polypeptide as translated from a coding open reading frame, or as processed to its mature form, while a polypeptide or peptide informally refers to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

The term electrophile as used herein refers to a chemical moiety that is capable of accepting an electron pair (e.g. Lewis acid, electron pair acceptor). For example, an electrophile as used herein is a chemical moiety that accepts a pair of electrons thereby forming a covalent bond.

The term “reversible covalent bond” as used herein refers to a labile bond between the amine of a lysine residue and a compound as disclosed herein (e.g. between an electron deficient functional group and the amine of the lysine reside). As used herein a reversible covalent inhibitor or reversible covalent modifier, refer to classes of compounds that comprise a reversible covalent bond. The reversible covalent bond may be a bond as described herein (e.g., carbon nitrogen double bond or sulfur-nitrogen single bond). Bandyopadhyay, A. et al. Curr Opin Chem Biol. October 2016, (34) pp. 110-116; Serafimova, I. M. et al. Nat Chem Biol. May 2012, 8(5), pp. 471-476; and Bradshaw M. J. et al. Nat Chem Biol. Jul. 2015, 11(7): 525-531, describe reversible covalent modifiers or reversible covalent chemical kinetics, the entire contents of each of which are incorporated herein by reference.

The term “contacting” may include allowing two species to react, interact, or physically touch, for example the two species may be an exogenous compound and an AKT1 protein or mutant thereof as described herein.

As used herein “cancer” in a subject refers to the presence of cells possessing characteristics typical of cancer-causing cells, such as uncontrolled proliferation, immortality, metastatic potential, rapid growth and proliferation rate, and certain characteristic morphological features. In some circumstances, cancers will be in the form of a tumor, or such cells that may exist locally within a subject, or circulate in the blood stream as independent cells.

The term “tumor” can refer to a solid or fluid-filled lesion or structure that may be formed by cancerous or non-cancerous cells, such as cells exhibiting aberrant cell growth or division. The terms “mass” and “nodule” are often used synonymously with “tumor”. Tumors include malignant tumors or benign tumors. An example of a malignant tumor can be a carcinoma which is known to comprise transformed cells.

The term “in vivo” is used to describe an event that takes place in a subject's body.

The term “ex vivo” is used to describe an event that takes place outside of a subject's body. An “ex vivo” assay is not performed on a subject. Rather, it is performed upon a sample separate from a subject. An example of an ‘ex vivo’ assay performed on a sample is an ‘in vitro’ assay.

The term “in vitro” is used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms ““administer”, “administered”, “administers” and “administering” a compound should be understood to mean providing a compound of the invention or a prodrug of a compound of the invention to the individual in need.

The terms “determining”, “measuring”, “evaluating”, “assessing, ““assaying, “and “analyzing” are often used interchangeably herein to refer to forms of measurement, and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or thereof.

The term “kinase activity” as herein refers to a variance in the nucleotide sequence of agene that results in a modulated kinase activity (increased or decreased). The modulated kinase activity is a result of the variance in the nucleic acid and is associated with the protein for which the gene encodes.

“Alkyl” refers to a straight or branched hydrocarbon chain monovalent radical consisting solely of carbon and hydrogen atoms, containing no unsaturation, and preferably having from one to twelve carbon atoms (i.e., C₁₋₁₂ alkyl). The alkyl is attached to the remainder of the molecule through a single bond. An alkyl chain may be optionally substituted by one or more substituents such as those substituents described herein. In certain embodiments, an alkyl comprises one to twelve carbon atoms (i.e., C₁₋₁₂ alkyl). In certain embodiments, an alkyl comprises one to eight carbon atoms (i.e., C₁₋₈ alkyl). In other embodiments, an alkyl comprises one to five carbon atoms (i.e., C₁₋₅ alkyl). In other embodiments, an alkyl comprises one to four carbon atoms (i.e., C₁₋₄ alkyl). In other embodiments, an alkyl comprises one to three carbon atoms (i.e., C₁₋₃ alkyl). In other embodiments, an alkyl comprises one to two carbon atoms (i.e., C₁₋₂ alkyl). In other embodiments, an alkyl comprises one carbon atom (i.e., C₁ alkyl). In other embodiments, an alkyl comprises five to fifteen carbon atoms (i.e., C₅₋₁₅ alkyl). In other embodiments, an alkyl comprises five to eight carbon atoms (i.e., C₅₋₈ alkyl). In other embodiments, an alkyl comprises two to five carbon atoms (i.e., C₂₋₅ alkyl). In other embodiments, an alkyl comprises three to five carbon atoms (i.e., C₃₋₅ alkyl). For example, the alkyl group may be attached to the rest of the molecule by a single bond, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂₋₁₂ alkenyl). An alkenyl chain may be optionally substituted by one or more substituents such as those substituents described herein. In certain embodiments, an alkenyl comprises two to eight carbon atoms (i.e., C₂₋₈ alkenyl). In certain embodiments, an alkenyl comprises two to six carbon atoms (i.e., C₂₋₆ alkenyl). In other embodiments, an alkenyl comprises two to four carbon atoms (i.e., C₂₋₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon—carbon triple bond, and preferably having from two to twelve carbon atoms (i.e., C₂₋₁₂ alkynyl). An alkylnyl chain may be optionally substituted by one or more substituents such as those substituents described herein. In certain embodiments, an alkynyl comprises two to eight carbon atoms (i.e., C₂₋₈ alkynyl). In other embodiments, an alkynyl comprises two to six carbon atoms (i.e., C₂₋₆ alkynyl). In other embodiments, an alkynyl comprises two to four carbon atoms (i.e., C₂₋₄ alkynyl). The alkynyl is attached to the rest of the molecule by a single bond, for example, ethynyl, propynyl, butynyl, pentynyl, hexynyl, and the like.

“Alkylene” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, (methyl)ethylene, butylene, and the like. The alkylene chain is attached to the rest of the molecule through a single bond and to the radical group through a single bond. An alkylene chain may be optionally substituted by one or more substituents such as those substituents described herein. In certain embodiments, an alkylene comprises one to ten carbon atoms (i.e., C₁₋₁₀ alkylene). In certain embodiments, an alkylene comprises one to eight carbon atoms (i.e., C₁₋₈ alkylene). In other embodiments, an alkylene comprises one to five carbon atoms (i.e., C₁₋₅ alkylene). In other embodiments, an alkylene comprises one to four carbon atoms (i.e., C₁₋₄ alkylene). In other embodiments, an alkylene comprises one to three carbon atoms (i.e., C₁₋₃ alkylene). In other embodiments, an alkylene comprises one to two carbon atoms (i.e., C₁₋₂ alkylene). In other embodiments, an alkylene comprises one carbon atom (i.e., C₁ alkylene). In other embodiments, an alkylene comprises five to eight carbon atoms (i.e., C₅₋₈ alkylene). In other embodiments, an alkylene comprises two to five carbon atoms (i.e., C₂₋₅ alkylene). In other embodiments, an alkylene comprises three to five carbon atoms (i.e., C₃₋₅ alkylene).

“Alkenylene” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. An alkenylene chain may be optionally substituted by one or more substituents such as those substituents described herein. In certain embodiments, an alkenylene comprises two to ten carbon atoms (i.e., C₂₋₁₀ alkenylene). In certain embodiments, an alkenylene comprises two to eight carbon atoms (i.e., C₂₋₈ alkenylene). In other embodiments, an alkenylene comprises two to five carbon atoms (i.e., C₂₋₅ alkenylene). In other embodiments, an alkenylene comprises two to four carbon atoms (i.e., C₂₋₄ alkenylene). In other embodiments, an alkenylene comprises two to three carbon atoms (i.e., C₂₋₃ alkenylene). In other embodiments, an alkenylene comprises two carbon atoms (i.e., C₂ alkenylene). In other embodiments, an alkenylene comprises five to eight carbon atoms (i.e., C₅₋₈ alkenylene). In other embodiments, an alkenylene comprises three to five carbon atoms (i.e., C₃₋₅ alkenylene).

“Alkynylene” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. An alkynylene chain may be optionally substituted by one or more substituents such as those substituents described herein. In certain embodiments, an alkynylene comprises two to ten carbon atoms (i.e., C₂₋₁₀ alkynylene). In certain embodiments, an alkynylene comprises two to eight carbon atoms (i.e., C₂₋₈ alkynylene). In other embodiments, an alkynylene comprises two to five carbon atoms (i.e., C₂₋₅ alkynylene). In other embodiments, an alkynylene comprises two to four carbon atoms (i.e., C₂₋₄ alkynylene). In other embodiments, an alkynylene comprises two to three carbon atoms (i.e., C₂₋₃ alkynylene). In other embodiments, an alkynylene comprises two carbon atoms (i.e., C₂ alkynylene). In other embodiments, an alkynylene comprises five to eight carbon atoms (i.e., C₅₋₈ alkynylene). In other embodiments, an alkynylene comprises three to five carbon atoms (i.e., C₃₋₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to saturated hydrocarbon groups, including straight-chain alkyl and branched-chain alkyl groups that contain from 1 to 6 carbons. The term -C_{x-y} alkylene-refers to a alkylene chain with from x to y carbons in the alkylene chain. For example, —C₁₋₆ alkylene-may be selected from methylene, ethylene, propylene, butylene, pentylene, and hexylene, any one of which may be optionally substituted.

The terms “C_{x-y} alkenyl” and “C_{x-y} alkynyl” refer to unsaturated aliphatic groups analogous in length and possible substitution to the alkyls described above, but that contain at least one double or triple bond, respectively. The term -C_{x-y} alkenylene-refers to a alkenylene chain with from x to y carbons in the alkenylene chain. For example, —C₂₋₆ alkenylene-may be selected from ethenylene, propenylene, butenylene, pentenylene, and hexenylene, any one of which may be optionally substituted. An alkenylene chain may have one double bond or more than one double bond in the alkenylene chain. The term -C_{x-y} alkynylene-refers to a alkynylene chain with from x to y carbons in the alkynylene chain. For example, —C₂₋₆ alkynylene-may be selected from ethynylene, propynylene, butynylene, pentynylene, and hexynylene, any one of which may be optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle includes 3- to 10-membered monocyclic rings and polycyclic rings (e.g., 6- to 12-membered bicyclic rings). Each ring of a polycyclic carbocycle may be selected from saturated, unsaturated, and aromatic rings. Polycyclic carbocycles may be fused, bridged or spiro-ring systems. Each ring of a bicyclic carbocycle may be selected from saturated, unsaturated, and aromatic rings. Bicyclic carbocycles may be fused, bridged or spiro-ring systems. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

The term “carbocyclene” as used herein refers to a divalent saturated, unsaturated or aromatic ring in which each atom of the ring is carbon. The carbocyclene is attached to the rest of the molecule through a single bond and to the radical group through a single bond. A carbocyclene may be optionally substituted by one or more substituents such as those substituents described herein. Carbocyclene includes divalent 3- to 10-membered monocyclic rings and divalent polycyclic rings (e.g., 6- to 12-membered bicyclic rings). Each ring of a polycyclic carbocyclene may be selected from saturated, unsaturated, and aromatic rings. Polycyclic carbocyclenes may be fused, bridged or spiro-ring systems. Polycyclic carbocyclenes may be fused, bridged or spiro-ring systems. The single bond connecting the carbocyclene to the rest of the molecule and the single bond connecting the carbocyclene to the radical group may be located on the same ring or different rings of a polycyclic carbocyclene. In some embodiments, the carbocycle is an arylene, for example, a phenylene. A “phenylene” as used herein refers to a divalent benzene group. The phenylene is attached to the rest of the molecule through a single bond and to the radical group through a single bond. A phenylene may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused, bridged, or spiro-ring systems, and preferably having from three to twelve carbon atoms (i.e., C₃₋₁₂ cycloalkyl). In certain embodiments, a cycloalkyl comprises three to ten carbon atoms (i.e., C₃₋₁₀ cycloalkyl). In other embodiments, a cycloalkyl comprises five to seven carbon atoms (i.e., C₅₋₇ cycloalkyl). The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbomyl (i.e., bicyclo[2.2.1]heptanyl), norbornenyl, decalinyl, 7,7-dimethyl-bicyclo[2.2.1]heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e., C₃₋₁₂ cycloalkenyl). In certain embodiments, a cycloalkenyl comprises three to ten carbon atoms (i.e., C₃₋₁₀ cycloalkenyl). In other embodiments, a cycloalkenyl comprises five to seven carbon atoms (i.e., C₅₋₇ cycloalkenyl). The cycloalkenyl may be attached to the rest of the molecule by a single bond. Examples of monocyclic cycloalkenyls include, e.g., cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic polycyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) p-electron system in accordance with the Huckel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring. For example, the term “C₃₋₆ carbocycle” can be a saturated, unsaturated or aromatic ring system that contains from 3 to 6 carbon atoms—any one of which may be optionally substituted as provided herein.

The term “heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and polycyclic rings (e.g., 6- to 12-membered bicyclic rings). Polycyclic heterocycles may be fused, bridged or spiro-ring systems. Each ring of a polycyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

The term “heterocyclene” as used herein refers to a divalent saturated, unsaturated, non-aromatic or aromatic ring comprising one or more heteroatoms. Exemplary heteroatoms include N, O, Si, P, B, and S atoms. The heterocyclene is attached to the rest of the molecule through a single bond and to the radical group through a single bond. The single bond attaching the heterocyclene group to the rest of the molecule and the single bond attaching the heterocyclene group to the radical group may be each independently connected through any atom of the heterocyclene as valency permits, including a carbon atom in the heterocyclene ring or a heteroatom in the heterocyclene ring. A heterocyclene may be optionally substituted by one or more substituents such as those substituents described herein. Heterocyclenes include 3- to 10-membered monocyclic rings and polycyclic rings (e.g., 6- to 12-membered bicyclic rings). Each ring of a polycyclic heterocyclene may be selected from saturated, unsaturated, and aromatic rings. Polycyclic heterocyclenes may be fused, bridged or spiro-ring systems. The single bond connecting the heterocyclene to the rest of the molecule and the single bond connecting the heterocyclene to the radical group may be located on the same ring or different rings of a polycyclic heterocyclene, and may be attached to the rest of the molecule or the radical group through any atom of the heterocyclene, valence permitting, such as a carbon or nitrogen atom of the heterocycle. In some embodiments, the heterocyclene comprises at least one heteroatom selected from oxygen, nitrogen, sulfur, or any combination thereof. In some embodiments, the heterocyclene comprises at least one heteroatom selected from oxygen, nitrogen, or any combination thereof. In some embodiments, the heterocyclene comprises at least one heteroatom selected from oxygen, sulfur, or any combination thereof. In some embodiments, the heterocyclene comprises at least one heteroatom selected from nitrogen, sulfur, or any combination thereof. In some embodiments, the heterocyclene is a heteroarylene. In some embodiments, the heterocyclene is a heterocycloalkylene.

“Heterocycloalkyl” refers to a stable 3 to 12 membered non-aromatic ring radical that comprises two to twelve carbon atoms and at least one heteroatom wherein each heteroatom may be selected from N, O, Si, P, B, and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused, bridged, or spiro-ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxothiomorpholinyl, and 1,1-dioxothiomorpholinyl. Heterocycloalkyl may be optionally substituted by one or more substituents such as those substituents described herein.

The term “heteroaryl” refers to a radical derived from a 3- to 12-membered aromatic ring radical that comprises one to eleven carbon atoms and at least one heteroatom wherein each heteroatom may be selected from N, O, and S. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. As used herein, the heteroaryl ring may be selected from monocyclic or bicyclic and fused or bridged ring systems wherein at least one of the rings in the ring system is aromatic, i.e., it contains a cyclic, delocalized (4n+2) p-electron system in accordance with the Hückel theory. The heteroatom(s) in the heteroaryl radical may be optionally oxidized. One or more nitrogen atoms, if present, are optionally quatemized. The heteroaryl may be attached to the rest of the molecule through any atom of the heteroaryl, valence permitting, such as a carbon or nitrogen atom of the heteroaryl. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl may be optionally substituted by one or more substituents such as those substituents described herein.

An “X-membered heterocycle” refers to the number of endocyclic atoms, i.e., X, in the ring. For example, a 5-membered heteroaryl ring or 5-membered aromatic heterocycle has 5 endocyclic atoms, e.g., triazole, oxazole, thiophene, etc.

“Alkoxy” refers to a radical bonded through an oxygen atom of the formula: O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, and I). When an alkyl group is substituted with more than one halogen radical, each halogen may be independently selected for example, 1-chloro,2-fluoroethane.

The term “substituted” refers to moieties having substituents replacing a hydrogen on one or more carbons or substitutable heteroatoms, e.g., an NH or NH₂ of a compound. Unless specified otherwise (e.g., by using the terms “substituted” or “optionally substituted”, or by the inclusion of an “—R” group), chemical groups described herein are unsubstituted. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. Unless specified otherwise (e.g., by the phrase “two or more substituents come together to form a carbocycle or heterocycle”), two or more substituents on the same or different substituted carbon or substitutable heteroatom will not come together to form a ring structure (e.g., two substituents on an alkyl chain forming a monocyclic ring, or two substituents on a ring system to form a spiro, fused, or bridged polycyclic ring system). As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)R^{a}, —R^{b}—OC(O)OR^{a}, —R^{b}—OC(O)N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 0, 1, or 2), —R^{b}—S(O)ₜOR^{a} (where t is 1 or 2), —R^{b}—S(O)ₜN(R^{a})₂ (where t is 1 or 2), and —P(O)(R^{a})₂; and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any one of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo(═N—OH), hydrazine(═N—NH₂), —R^{b}—OR^{a}, —R^{b}—OC(O)R^{a}, —R^{b}—OC(O)OR^{a}, —R^{b}—OC(O)N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 0, 1, or 2), —R^{b}—S(O)OR^{a} (where t is 1 or 2), —R^{b}—S(O)ₜN(R^{a})₂ (where t is 1 or 2), and —P(O)(R^{a})₂; wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a},
—R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}, —C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 0, 1, or 2), —R^{b}—S(O)OR^{a} (where t is 1 or 2), —R^{b}—S(O)ₜN(R^{a})₂ (where t is 1 or 2), and —P(O)(R^{a})₂; and wherein each R^{b} is independently selected from a direct bond or a straight or branched alkylene, alkenylene, or alkynylene chain, and each R^{c} is a straight or branched alkylene, alkenylene or alkynylene chain. It will be understood by those skilled in the art that substituents can themselves be substituted, if appropriate.

In some embodiments, “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂CI, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted alkenyl (e.g., C₁-C₈ alkylenyl, C₁-C₆ alkylenyl, or C₁-C₄ alkylenyl), unsubstituted alkynyl (e.g., C₁-C₈ alkynyl, C₁-C₆ alkynyl, or C₁-C₄ alkynyl), unsubstituted heteroalkyl (e.g., 2 to 8 membered heteroalkyl, 2 to 6 membered heteroalkyl, or 2 to 4 membered heteroalkyl), unsubstituted cycloalkenyl (e.g., C₃-C₈ cycloalkenyl, C₃-C₆ cycloalkenyl, or C₅-C₆ cycloalkenyl), unsubstituted carbocycle (e.g., C₃-C₈ carbocycle, C₃-C₆ carbocycle, or C₅-C₆ carbocycle), unsubstituted cycloalkyl (e.g., C₃-C₈ cycloalkyl, C₃-C₆ cycloalkyl, or C₅-C₆ cycloalkyl), unsubstituted heterocycle (e.g., 3 to 8 membered heterocycle, 3 to 6 membered heterocycle, or 5 to 6 membered heterocycle), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂CI, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted alkenyl (e.g., C₁-C₈ alkylenyl, C₁-C₆ alkylenyl, or C₁-C₄ alkylenyl), unsubstituted alkynyl (e.g., C₁-C₈ alkynyl, C₁-C₆ alkynyl, or C₁-C₄ alkynyl), unsubstituted heteroalkyl (e.g., 2 to 8 membered heteroalkyl, 2 to 6 membered heteroalkyl, or 2 to 4 membered heteroalkyl), unsubstituted cycloalkenyl (e.g., C₃-C₈ cycloalkenyl, C₃-C₆ cycloalkenyl, or C₅-C₆ cycloalkenyl), unsubstituted carbocycle (e.g., C₃-C₈ carbocycle, C₃-C₆ carbocycle, or C₅-C₆ carbocycle), unsubstituted cycloalkyl (e.g., C₃-C₈ cycloalkyl, C₃-C₆ cycloalkyl, or C₅-C₆ cycloalkyl), unsubstituted heterocycle (e.g., 3 to 8 membered heterocycle, 3 to 6 membered heterocycle, or 5 to 6 membered heterocycle), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), alkenyl (e.g., C₁-C₈ alkylenyl, C₁-C₆ alkylenyl, or C₁-C₄ alkylenyl), alkynyl (e.g., C₁-C₈ alkynyl, C₁-C₆ alkynyl, or C₁-C₄ alkynyl), heteroalkyl (e.g., 2 to 8 membered heteroalkyl, 2 to 6 membered heteroalkyl, or 2 to 4 membered heteroalkyl), cycloalkenyl (e.g., C₃-C₈ cycloalkenyl, C₃-C₆ cycloalkenyl, or C₅-C₆ cycloalkenyl), carbocycle (e.g., C₃-C₈ carbocycle, C₃-C₆ carbocycle, or C₅-C₆ carbocycle), cycloalkyl (e.g., C₃-C₈ cycloalkyl, C₃-C₆ cycloalkyl, or C₅-C₆ cycloalkyl), heterocycle (e.g., 3 to 8 membered heterocycle, 3 to 6 membered heterocycle, or 5 to 6 membered heterocycle), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), or heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂CI, —OCH₂Br, —OCH₂I, —OCH₂F, —CN, —OH, —NH₂, —COOH, —CONH₂, —N O₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted alkenyl (e.g., C₁-C₈ alkylenyl, C₁-C₆ alkylenyl, or C₁-C₄ alkylenyl), unsubstituted alkynyl (e.g., C₁-C₈ alkynyl, C₁-C₆ alkynyl, or C₁-C₄ alkynyl), unsubstituted heteroalkyl (e.g., 2 to 8 membered heteroalkyl, 2 to 6 membered heteroalkyl, or 2 to 4 membered heteroalkyl), unsubstituted cycloalkenyl (e.g., C₃-C₈ cycloalkenyl, C₃-C₆ cycloalkenyl, or C₅-C₆ cycloalkenyl), unsubstituted carbocycle (e.g., C₃-C₈ carbocycle, C₃-C₆ carbocycle, or C₅-C₆ carbocycle), unsubstituted cycloalkyl (e.g., C₃-C₈ cycloalkyl, C₃-C₆ cycloalkyl, or C₅-C₆ cycloalkyl), unsubstituted heterocycle (e.g., 3 to 8 membered heterocycle, 3 to 6 membered heterocycle, or 5 to 6 membered heterocycle), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), alkenyl (e.g., C₁-C₈ alkylenyl, C₁-C₆ alkylenyl, or C₁-C₄ alkylenyl), alkynyl (e.g., C₁-C₈ alkynyl, C₁-C₆ alkynyl, or C₁-C₄ alkynyl), heteroalkyl (e.g., 2 to 8 membered heteroalkyl, 2 to 6 membered heteroalkyl, or 2 to 4 membered heteroalkyl), cycloalkenyl (e.g., C₃-C₈ cycloalkenyl, C₃-C₆ cycloalkenyl, or C₅-C₆ cycloalkenyl), carbocycle (e.g., C₃-C₈ carbocycle, C₃-C₆ carbocycle, or C₅-C₆ carbocycle), cycloalkyl (e.g., C₃-C₈ cycloalkyl, C₃-C₆ cycloalkyl, or C₅-C₆ cycloalkyl), heterocycle (e.g., 3 to 8 membered heterocycle, 3 to 6 membered heterocycle, or 5 to 6 membered heterocycle), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), or heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂CI, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted alkenyl (e.g., C₁-C₈ alkylenyl, C₁-C₆ alkylenyl, or C₁-C₄ alkylenyl), unsubstituted alkynyl (e.g., C₁-C₈ alkynyl, C₁-C₆ alkynyl, or C₁-C₄ alkynyl), unsubstituted heteroalkyl (e.g., 2 to 8 membered heteroalkyl, 2 to 6 membered heteroalkyl, or 2 to 4 membered heteroalkyl), unsubstituted cycloalkenyl (e.g., C₃-C₈ cycloalkenyl, C₃-C₆ cycloalkenyl, or C₅-C₆ cycloalkenyl), unsubstituted carbocycle (e.g., C₃-C₈ carbocycle, C₃-C₆ carbocycle, or C₅-C₆ carbocycle), unsubstituted cycloalkyl (e.g., C₃-C₈ cycloalkyl, C₃-C₆ cycloalkyl, or C₅-C₆ cycloalkyl), unsubstituted heterocycle (e.g., 3 to 8 membered heterocycle, 3 to 6 membered heterocycle, or 5 to 6 membered heterocycle), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted alkenyl is a substituted or unsubstituted C₁-C₂₀ alkenyl, each substituted or unsubstituted alkynyl is a substituted or unsubstituted C₁-C₂₀ alkynyl, each substituted or unsubstituted carbocycle is a substituted or unsubstituted C₃-C₈ carbocycle, each substituted or unsubstituted cycloalkenyl is a substituted or unsubstituted C₃-C₈ cycloalkenyl, each substituted or unsubstituted heterocycle is a substituted or unsubstituted 3 to 8 membered heterocycle, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, each substituted or unsubstituted alkenyl is a substituted or unsubstituted C₁-C₈ alkenyl, each substituted or unsubstituted alkynyl is a substituted or unsubstituted C₁-C₈ alkynyl, each substituted or unsubstituted carbocycle is a substituted or unsubstituted C₃-C₇ carbocycle, each substituted or unsubstituted cycloalkenyl is a substituted or unsubstituted C₃-C₇ cycloalkenyl, each substituted or unsubstituted heterocycle is a substituted or unsubstituted 5 to 6 membered heterocycle, and each substituted or unsubstituted heteroaryl is a substituted or unsubstituted 5 to 6 membered heteroaryl.

In some embodiments, each substituted group (e.g. substituted alkyl, alkenyl, alkynyl, heteroalkyl, carbocycle, cycloalkyl, cycloalkenyl, aryl, heteroaryl, heterocycle, heterocycloalkalkylene, alkenylene, alkynylene, heteroalkylene, carbocyclene, arylene, heterarylene and/or heterocyclene) described in the compounds herein is substituted with at least one substituent group. In other embodiments, at least one or all of these groups are substituted with at least one size-limited substituent group. In other embodiments, at least one or all of these groups are substituted with at least one lower substituent group.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### AKT1 Protein

In some aspects, the present disclosure provides an AKT1 protein covalently bound to a compound, wherein the compound is covalently bound to a lysine residue of the AKT1 protein. In some embodiments, the compound is exogenous. In some embodiments, the exogenous compound is selected from an exogenous AKT1 inhibitor and an exogenous AKT1 activator. In some embodiments, the exogenous compound is an exogenous AKT1 modulator. In some embodiments, the exogenous compound is an exogenous AKT1 inhibitor.

In some embodiments, the AKT1 protein is selected from a wild-type AKT1 protein and a mutated AKT1 protein. In some embodiments, the AKT1 protein is a mutated AKT1 protein. In some embodiments, the mutated AKT1 protein comprises a mutation selected from a E17K mutation, a E40K mutation, and a E49K mutation. In some embodiments, the mutated AKT1 protein comprises a E17K mutation. In some embodiments, the mutated AKT1 protein comprises a E40K mutation. In some embodiments, the mutated AKT1 protein comprises a E49K mutation.

In some embodiments, the exogenous compound is in contact a lysine residue of the AKT1 protein as described herein. In some embodiments, the contact is between the lysine reside of the AKT1 protein and the exogenous compound is a covalent bond. In some embodiments, the lysine reside is selected from K17, K40, K49, K158, K163, K179, K276, and K297. In some embodiments, the lysine residue is K17. In some embodiments, the lysine residue is selected from K158, K163, and K179. In some embodiments, the lysine residue is K158. In some embodiments, the lysine residue is K163. In some embodiments, the lysine residue is K179. In some embodiments, the lysine residue is selected from K40, K49, K276, and K297. In some embodiments, the lysine residue is K40. In some embodiments, the lysine residue is K49. In some embodiments, the lysine residue is K276. In some embodiments, the lysine residue is K297. In some embodiments, the lysine residue is selected from K17 and K297.

In some embodiments, the covalent bond between the exogenous compound and the lysine residue is a reversible covalent bond. In some embodiments, the reversible covalent bond is selected from a single bond, a double bound, and a triple bond. In some embodiments, the reversible covalent bond is a single bond. In some embodiments the reversible covalent bond is a double bond. In some embodiments, the reversible covalent bond is a triple bond. In some embodiments, the reversible covalent bond is a double bond between a carbon atom on the exogenous compound and the nitrogen atom on the sidechain of the lysine residue.

In some embodiments, the covalent bond between the exogenous compound and the lysine residue is a reversible covalent bond, wherein the lysine residue is selected from K17, K40, K49, K158, K163, K179, K276, and K297. In some embodiments, the covalent bond between the exogenous compound and the lysine residue is a reversible covalent bond selected from a single bond, a double bound, and a triple bond, wherein the lysine residue is selected from K17, K40, K49, K158, K163, K179, K276, and K297. In some embodiments, the covalent bond between the exogenous compound and the lysine residue is a reversible covalent single bond, wherein the lysine residue is selected from K17, K40, K49, K158, K163, K179, K276, and K297. In some embodiments, the covalent bond between the exogenous compound and the lysine residue is a reversible covalent double bond, wherein the lysine residue is selected from K17, K40, K49, K158, K163, K179, K276, and K297.

In some embodiments, the covalent bond between the exogenous compound and the lysine residue is a reversible covalent bond, wherein the lysine residue is selected from K17, K40, and K49. In some embodiments, the covalent bond between the exogenous compound and the lysine residue is a reversible covalent bond, wherein the lysine residue is K17. In some embodiments, the covalent bond between the exogenous compound and the lysine residue is a reversible covalent bond selected from a single bond, a double bound, and a triple bond, wherein the lysine residue is K17. In some embodiments, the covalent bond between the exogenous compound and the lysine residue is a reversible covalent single bond, wherein the lysine residue is K17. In some embodiments, the covalent bond between the exogenous compound and the lysine residue is a reversible covalent double bond, wherein the lysine residue is K17.

In some embodiments, the reversible covalent bond in the in vivo AKT1 protein comprises a carbon-nitrogen interaction. In some embodiments, the carbon-nitrogen interaction is selected form a carbon-nitrogen single bond and a carbon-nitrogen double bond. In some embodiments, the carbon-nitrogen interaction is a carbon-nitrogen single bond. In some embodiments, the carbon-nitrogen interaction is a carbon-nitrogen double bond.

In some embodiments, the AKT1 protein is covalently bound with the exogenous compound, wherein the exogenous compound is bound at only one residue of the AKT1 protein. In some embodiments, the AKT1 protein is covalently bond with the exogenous compound via one covalent bond. In some embodiments, the AKT1 protein is covalently bound with the exogenous compound, wherein the exogenous compound is bound at one lysine residue. In some embodiments, the AKT1 protein has a single covalent bond between a lysine residue and the exogenous compound. In some embodiments, the AKT1 protein has a single covalent bond between K17 and the exogenous compound. In some embodiments, the AKT1 protein has a single covalent bond between K158 and the exogenous compound. In some embodiments, the AKT1 protein has a single covalent bond between K163 and the exogenous compound. In some embodiments, the AKT1 protein has a single covalent bond between K179 and the exogenous compound.

In some embodiments, the exogenous compound has reduced engagement at other lysine residues when covalently bound at a lysine residue selected from K17, K40, K49, K158, K163, K179, K276, and K297. In some embodiments, the exogenous compound is in contact with one lysine residue selected from K17, K40, K49, K158, K163, K179, K276, and K297, and has reduced engagement at the remaining lysine residues. In some embodiments, the exogenous compound is in contact with K17, and has reduced engagement at K40, K49, K158, K163, K179, K276, or K297. In some embodiments, the exogenous compound is in contact with K158, and has reduced engagement at K17, K40, K49, K163, K179, K276, or K297. In some embodiments, the exogenous compound is in contact with K163, and has reduced engagement at K17, K40, K49, K158, K179, K276, or K297. In some embodiments, the exogenous compound is in contact with K179, and has reduced engagement at K17, K40, K49, K158, K163, K276, or K297. In some embodiments, the exogenous compound is in contact with K40, and has reduced engagement at K17, K49, K158, K163, K179, K276, or K297. In some embodiments, the exogenous compound is in contact with K49, and has reduced engagement at K17, K40, K158, K163, K179, K276, or K297. In some embodiments, the exogenous compound is in contact with K276, and has reduced engagement at K17, K40, K49, K158, K163, K179, or K297. In some embodiments, the exogenous compound is in contact with K297, and has reduced engagement at K17, K40, K49, K158, K163, K179, or K276.

In some embodiments, the AKT1 protein has a single lysine residue covalently bound with the exogenous compound. In some embodiments, the AKT1 protein has a single lysine residue covalently bound with the exogenous compound, wherein the single lysine residue is selected from K17, K40, K49, K158, K163, K179, K276, and K297. In some embodiments, the AKT1 protein has a single lysine residue covalently bound with the exogenous compound, wherein the single lysine residue is K17. In some embodiments, the AKT1 protein has a single lysine residue covalently bound with the exogenous compound, wherein the single lysine residue is K158. In some embodiments, the AKT1 protein has a single lysine residue covalently bound with the exogenous compound, wherein the single lysine residue is K163. In some embodiments, the AKT1 protein has a single lysine residue covalently bound with the exogenous compound, wherein the single lysine residue is K179. In some embodiments, the AKT1 protein has a single lysine residue covalently bound with the exogenous compound, wherein the single lysine residue is K40. In some embodiments, the AKT1 protein has a single lysine residue covalently bound with the exogenous compound, wherein the single lysine residue is K49. In some embodiments, the AKT1 protein has a single lysine residue covalently bound with the exogenous compound, wherein the single lysine residue is K276. In some embodiments, the AKT1 protein has a single lysine residue covalently bound with the exogenous compound, wherein the single lysine residue is K297.

In some embodiments, the AKT1 protein is in vivo. In some embodiments, the AKT1 protein is in vitro. In some embodiments, the AKT1 protein is ex vivo. In some embodiments, the AKT1 protein is an in vivo engineered protein.

In some embodiments, the AKT1 protein is an in vivo engineered AKT1 protein, wherein the in vivo engineered AKT1 protein is generated by contacting the AKT1 protein in vivo with the exogenous compound. In some embodiments, the AKT1 protein is a mammalian in vivo engineered AKT1 protein, wherein the in vivo engineered AKT1 protein is generated by contacting the AKT1 protein in vivo with the exogenous compound. In some embodiments, the AKT1 protein is a human in vivo engineered AKT1 protein, wherein the in vivo engineered AKT1 protein is generated by contacting the AKT1 protein in vivo with the exogenous compound.

In some embodiments, the reversible covalent bond in the in vivo AKT1 protein is between a carbon atom and a nitrogen atom. In some embodiments, the reversible covalent bond in the in vivo AKT1 protein is between a carbon atom of the exogenous compound and a nitrogen atom of the lysine residue. In some embodiments, the reversible covalent bond in the in vivo AKT1 protein is between a carbon atom of the exogenous compound and a nitrogen atom of the lysine residue selected from K17, K40, K49, K158, K163, K179, K276, and K297. In some embodiments, the reversible covalent bond in the in vivo AKT1 protein is between a carbon atom of the exogenous compound and a nitrogen atom of the K17 lysine residue. In some embodiments, the reversible covalent bond in the in vivo AKT1 protein is between a carbon atom of the exogenous compound and a nitrogen atom of the K158 lysine residue. In some embodiments, the reversible covalent bond in the in vivo AKT1 protein is between a carbon atom of the exogenous compound and a nitrogen atom of the K163 lysine residue. In some embodiments, the reversible covalent bond in the in vivo AKT1 protein is between a carbon atom of the exogenous compound and a nitrogen atom of the K179 lysine residue.

In some embodiments, the reversible covalent bond in the in vivo AKT1 protein is a carbon-nitrogen double bond. In some embodiments, the reversible covalent bond in the in vivo AKT1 protein is a carbon-nitrogen double bond between the exogenous compound and the lysine residue. In some embodiments, the carbon-nitrogen double bond is between the exogenous compound and the lysine residue, wherein the lysine residue is selected from K17, K40, K49, K158, K163, K179, K276, and K297. In some embodiments, the carbon-nitrogen double bond is between the exogenous compound and the K17 lysine residue. In some embodiments, the carbon-nitrogen double bond is between the exogenous compound and the K158 lysine residue. In some embodiments, the carbon-nitrogen double bond is between the exogenous compound and the K163 lysine residue. In some embodiments, the carbon-nitrogen double bond is between the exogenous compound and the K179 lysine residue. In some embodiments, the carbon-nitrogen double bond is between the exogenous compound and the K40 lysine residue. In some embodiments, the carbon-nitrogen double bond is between the exogenous compound and the K49 lysine residue. In some embodiments, the carbon-nitrogen double bond is between the exogenous compound and the K276 lysine residue. In some embodiments, the carbon-nitrogen double bond is between the exogenous compound and the K297 lysine residue.

In some embodiments, the carbon-nitrogen double bond is an imine bound. In some embodiments, the imine bond is between a carbon atom of the exogenous compound and a nitrogen atom of the lysine residue In some embodiments, the imine bond is between the exogenous compound and the lysine residue, wherein the lysine residue is selected from K17, K40, K49, K158, K163, K179, K276, and K297. In some embodiments, the imine bond is between the exogenous compound and the K 17 lysine residue. In some embodiments, the imine bond is between the exogenous compound and the K158 lysine residue. In some embodiments, the imine bond is between the exogenous compound and the K163 lysine residue. In some embodiments, the imine bond is between the exogenous compound and the K179 lysine residue. In some embodiments, the imine bond is between the exogenous compound and the K40 lysine residue. In some embodiments, the imine bond is between the exogenous compound and the K49 lysine residue. In some embodiments, the imine bond is between the exogenous compound and the K276 lysine residue. In some embodiments, the imine bond is between the exogenous compound and the K297 lysine residue.

In some embodiments, the exogenous compound comprises a functional group. In some embodiments, the exogenous compound comprises an aldehyde functional group. In some embodiments, the reversible covalent bond is between the aldehyde functional group and the lysine residue. In some embodiments, the reversible covalent bond is between the aldehyde functional group and the lysine residue selected from K17, K40, K49, K158, K163, K179, K276, and K297. In some embodiments, the reversible covalent bond is between the aldehyde functional group and the K17 lysine residue. In some embodiments, the reversible covalent bond is between the aldehyde functional group and the K158 lysine residue. In some embodiments, the reversible covalent bond is between the aldehyde functional group and the K163 lysine residue. In some embodiments, the reversible covalent bond is between the aldehyde functional group and the K179 lysine residue. In some embodiments, the reversible covalent bond is between the aldehyde functional group and the K40 lysine residue. In some embodiments, the reversible covalent bond is between the aldehyde functional group and the K49 lysine residue. In some embodiments, the reversible covalent bond is between the aldehyde functional group and the K276 lysine residue. In some embodiments, the reversible covalent bond is between the aldehyde functional group and the K297 lysine residue.

In some embodiments, the carbon-nitrogen bond is a reversible bond that results from a reversible reaction. In some embodiments, the carbon-nitrogen bond is a reversible bond that results from a reversible reaction between the exogenous compound and a lysine residue. In some embodiments, the carbon-nitrogen bond is a reversible bond that results from a reversible reaction between the exogenous compound and a lysine residue selected from K17, K40, K49, K158, K163, K179, K276, and K297. In some embodiments, the carbon-nitrogen bond is a reversible bond that results from a reversible reaction between the exogenous compound and the K17 lysine residue. In some embodiments, the carbon-nitrogen bond is a reversible bond that results from a reversible reaction between the exogenous compound and the K158 lysine residue. In some embodiments, the carbon-nitrogen bond is a reversible bond that results from a reversible reaction between the exogenous compound and the K163 lysine residue. In some embodiments, the carbon-nitrogen bond is a reversible bond that results from a reversible reaction between the exogenous compound and the K179 lysine residue. In some embodiments, the carbon-nitrogen bond is a reversible bond that results from a reversible reaction between the exogenous compound and the K40 lysine residue. In some embodiments, the carbon-nitrogen bond is a reversible bond that results from a reversible reaction between the exogenous compound and the K49 lysine residue. In some embodiments, the carbon-nitrogen bond is a reversible bond that results from a reversible reaction between the exogenous compound and the K276 lysine residue. In some embodiments, the carbon-nitrogen bond is a reversible bond that results from a reversible reaction between the exogenous compound and the K297 lysine residue.

In some embodiments, the carbon-nitrogen double bond results from a reversible reaction between the amine functional group of a lysine residue and an aldehyde functional group on the exogenous compound. In some embodiments, the carbon-nitrogen double bond results from a reversible reaction between the amine functional group of a lysine residue and an aldehyde functional group on the exogenous compound, wherein the lysine residue is selected from K17, K40, K49, K158, K163, K179, K276, and K297. In some embodiments, the carbon-nitrogen double bond results from a reversible reaction between the amine functional group of K17 and an aldehyde functional group on the exogenous compound. In some embodiments, the carbon-nitrogen double bond results from a reversible reaction between the amine functional group of K158 and an aldehyde functional group on the exogenous compound. In some embodiments, the carbon-nitrogen double bond results from a reversible reaction between the amine functional group of K163 and an aldehyde functional group on the exogenous compound. In some embodiments, the carbon-nitrogen double bond results from a reversible reaction between the amine functional group of K179 and an aldehyde functional group on the exogenous compound. In some embodiments, the carbon-nitrogen double bond results from a reversible reaction between the amine functional group of K40 and an aldehyde functional group on the exogenous compound. In some embodiments, the carbon-nitrogen double bond results from a reversible reaction between the amine functional group of K49 and an aldehyde functional group on the exogenous compound. In some embodiments, the carbon-nitrogen double bond results from a reversible reaction between the amine functional group of K276 and an aldehyde functional group on the exogenous compound. In some embodiments, the carbon-nitrogen double bond results from a reversible reaction between the amine functional group of K297 and an aldehyde functional group on the exogenous compound.

In some embodiments, the aldehyde functional group of the exogenous compound is an aromatic aldehyde. In some embodiments, the carbon-nitrogen double bond results from a reversible reaction between the amine functional group of a lysine residue and the aromatic aldehyde group on the exogenous compound. In some embodiments, the carbon-nitrogen double bond results from a reversible reaction between the amine functional group of a lysine residue and the aromatic aldehyde group on the exogenous compound, wherein the lysine residue is selected from K17, K40, K49, K158, K163, K179, K276, and K297. In some embodiments, the carbon-nitrogen double bond results from a reversible reaction between the amine functional group of K17 and the aromatic aldehyde group on the exogenous compound. In some embodiments, the carbon-nitrogen double bond results from a reversible reaction between the amine functional group of K158 and the aromatic aldehyde group on the exogenous compound. In some embodiments, the carbon-nitrogen double bond results from a reversible reaction between the amine functional group of K163 and the aromatic aldehyde group on the exogenous compound. In some embodiments, the carbon-nitrogen double bond results from a reversible reaction between the amine functional group of K179 and the aromatic aldehyde group on the exogenous compound. In some embodiments, the carbon-nitrogen double bond results from a reversible reaction between the amine functional group of K40 and the aromatic aldehyde group on the exogenous compound. In some embodiments, the carbon-nitrogen double bond results from a reversible reaction between the amine functional group of K49 and the aromatic aldehyde group on the exogenous compound. In some embodiments, the carbon-nitrogen double bond results from a reversible reaction between the amine functional group of K276 and the aromatic aldehyde group on the exogenous compound. In some embodiments, the carbon-nitrogen double bond results from a reversible reaction between the amine functional group of K297 and the aromatic aldehyde group on the exogenous compound.

In some embodiments, the AKT1 modulator is selected from N-(4-(2-(2-aminopyridin-3-yl)-5-(3-ethynylphenyl)-3H-imidazo[4,5-b]pyridin-3-yl)benzyl)-4-formyl-3-hydroxybenzamide (Compound A) and 4-(2-((4-(2-(2-aminopyridin-3-yl)-5-(3-ethynylphenyl)-3H-imidazo[4,5-b]pyridin-3-yl)benzyl)amino)ethyl)-2-hydroxybenzaldehyde (Compound B). In some embodiments, the AKT1 modulator is selected from

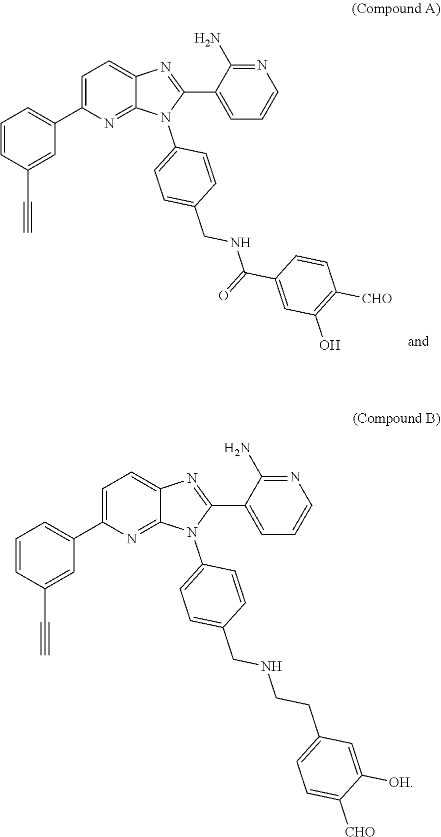 [SMILES: C#Cc1cccc(-c2ccc3nc(-c4cccnc4N)n(-c4ccc(CNC(=O)c5ccc(C=O)c(O)c5)cc4)c3n2)c1 ; C#Cc1cccc(-c2ccc3nc(-c4cccnc4N)n(-c4ccc(CNCCc5ccc(C=O)c(O)c5)cc4)c3n2)c1]

In some embodiments, the AKT1 modulator is Compound A. In some embodiments, the AKT1 modulator is Compound B.

In some embodiments, the AKT1 modulator is a compound or salt as disclosed herein. In some embodiments, the AKT1 modulator is selected from a compound or salt of Formula (I) or Formula (II). In some embodiments, the AKT1 modulator is selected from a compound or salt in Table 1.

In some embodiments, the AKT1 inhibitor is selected from N-(4-(2-(2-aminopyridin-3-yl)-5-(3-ethynylphenyl)-3H-imidazo[4,5-b]pyridin-3-yl)benzyl)-4-formyl-3-hydroxybenzamide (Compound A) and 4-(2-((4-(2-(2-aminopyridin-3-yl)-5-(3-ethynylphenyl)-3H-imidazo[4,5-b]pyridin-3-yl)benzyl)amino)ethyl)-2-hydroxybenzaldehyde (Compound B). In some embodiments, the AKT1 inhibitor is selected from

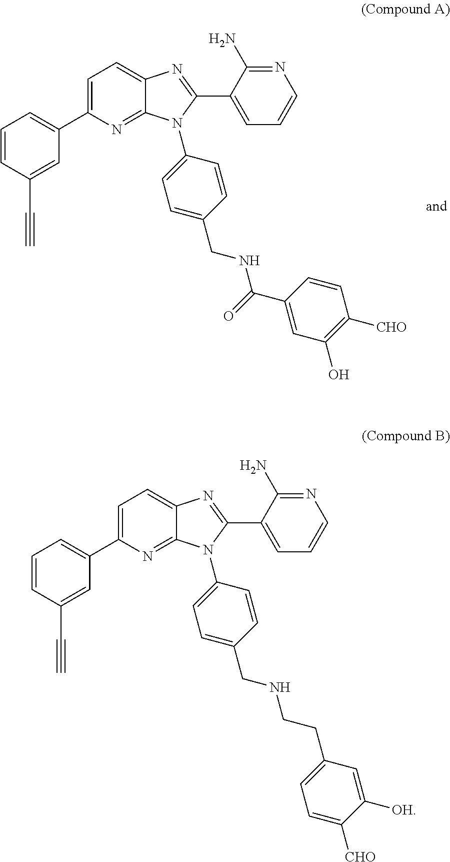 [SMILES: C#Cc1cccc(-c2ccc3nc(-c4cccnc4N)n(-c4ccc(CNC(=O)c5ccc(C=O)c(O)c5)cc4)c3n2)c1 ; C#Cc1cccc(-c2ccc3nc(-c4cccnc4N)n(-c4ccc(CNCCc5ccc(C=O)c(O)c5)cc4)c3n2)c1]

In some embodiments, the AKT1 inhibitor is Compound A. In some embodiments, the AKT1 inhibitor is Compound B.

In some embodiments, the AKT1 inhibitor is a compound or salt as disclosed herein. In some embodiments, the AKT1 inhibitor is selected from a compound or salt of Formula (I) or Formula (II). In some embodiments, the AKT1 inhibitor is selected from a compound or salt in Table 1.

In some embodiments, the exogenous compound is selected from N-(4-(2-(2-aminopyridin-3-yl)-5-(3-ethynylphenyl)-3H-imidazo[4,5-b]pyridin-3-yl)benzyl)-4-formyl-3-hydroxybenzamide (Compound A) and 4-(2-((4-(2-(2-aminopyridin-3-yl)-5-(3-ethynylphenyl)-3H-imidazo[4,5-b]pyridin-3-yl)benzyl)amino)ethyl)-2-hydroxybenzaldehyde (Compound B). In some embodiments, the exogenous compound is selected from

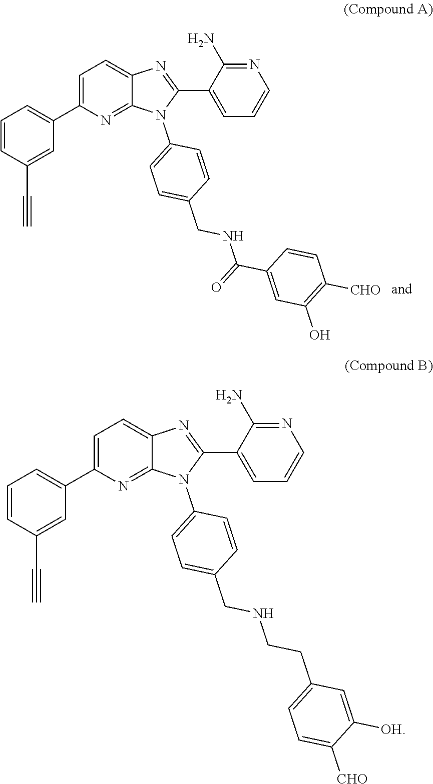 [SMILES: C#Cc1cccc(-c2ccc3nc(-c4cccnc4N)n(-c4ccc(CNC(=O)c5ccc(C=O)c(O)c5)cc4)c3n2)c1 ; C#Cc1cccc(-c2ccc3nc(-c4cccnc4N)n(-c4ccc(CNCCc5ccc(C=O)c(O)c5)cc4)c3n2)c1]

In some embodiments, the exogenous compound is Compound A. In some embodiments, the exogenous compound is Compound B.

In some embodiments, the exogenous compound is a compound or salt as disclosed herein. In some embodiments, the exogenous compound is selected from a compound or salt of Formula (I) or Formula (II). In some embodiments, the exogenous compound is selected from a compound or salt in Table 1.

In certain aspects the present disclosure provides an in vivo engineered AKT1 protein comprising a non-naturally occurring reversible covalent modification at a lysine residue, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered AKT1 protein is a human in vivo engineered AKT1 protein

In another aspect, the present disclosure provides an in vivo engineered AKT1 protein comprising a non-naturally occurring reversible covalent modification at a lysine residue selected from K17, K40, K49, K158, K163, K179, K276, and K297, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered AKT1 protein is a human in vivo engineered AKT1 protein

In another aspect, the present disclosure provides an in vivo engineered AKT1 protein comprising a non-naturally occurring reversible covalent modification at a lysine residue selected from K17, K40, K49, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered AKT1 protein is a human in vivo engineered AKT1 protein.

In another aspect, the present disclosure provides an in vivo engineered AKT1 protein comprising a non-naturally occurring reversible covalent modification at a K17 lysine residue, the reversible covalent modification being generated from an in vivo nucleophilic reaction between an exogenous aromatic aldehyde and the K17 lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered AKT1 protein is a human in vivo engineered AKT1 protein

In another aspect, the present disclosure provides an in vivo engineered AKT1 protein comprising a non-naturally occurring reversible covalent modification at a lysine residue selected from K158, K163, and K179, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered AKT1 protein is a human in vivo engineered AKT1 protein.

In another aspect, the present disclosure provides an in vivo engineered AKT1 protein comprising a non-naturally occurring reversible covalent modification at a lysine residue selected from K40, K49, K276 and K297, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered AKT1 protein is a human in vivo engineered AKT1 protein

In certain aspects the present disclosure provides a human in vivo engineered AKT1 protein comprising a non-naturally occurring reversible covalent modification at a lysine residue selected from K17, K40, K49, K158, K163, K179, K276, and K297, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In another aspect the present disclosure provides a human in vivo engineered AKT1 protein comprising a non-naturally occurring reversible covalent modification at a lysine residue selected from K17, K40, and K49, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In another aspect, the present disclosure provides a human in vivo engineered AKT1 protein comprising a non-naturally occurring reversible covalent modification at a K17 lysine residue, the reversible covalent modification being generated from an in vivo nucleophilic reaction between an exogenous aromatic aldehyde and the K17 lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In another aspect, the present disclosure provides a human in vivo engineered AKT1 protein comprising a non-naturally occurring reversible covalent modification at a lysine residue selected from K158, K163, and K179, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In another aspect, the present disclosure provides a human in vivo engineered AKT1 protein comprising a non-naturally occurring reversible covalent modification at a lysine residue selected from K40, K49, K276, and K297, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In certain aspects the present disclosure provides an in vivo engineered AKT1 protein comprises a mutation selected from E17K, E40K, and E49K, a non-naturally occurring reversible covalent modification at a lysine residue, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In certain aspects the present disclosure provides an in vivo engineered AKT1 protein comprising a E17K mutation, a non-naturally occurring reversible covalent modification at a lysine residue, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In another aspect, the present disclosure provides an in vivo engineered AKT1 protein comprising a E17K mutation, a non-naturally occurring reversible covalent modification at a lysine residue selected from K17, K40, K49, K158, K163, K179, K276, and K297, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue. In some embodiments, the in vivo engineered AKT1 protein is a human in vivo engineered AKT1 protein.

In certain aspects the present disclosure provides a human in vivo engineered AKT1 protein comprising a E17K mutation, a non-naturally occurring reversible covalent modification at a lysine residue, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

In another aspect, the present disclosure provides a human in vivo engineered AKT1 protein comprising a E17K mutation, a non-naturally occurring reversible covalent modification at a lysine residue, the reversible covalent modification being generated from an in vivo nucleophilic reaction between an exogenous aromatic aldehyde and the lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

### Method of Modifying AKT1 Proteins

In some aspects the present disclosure provides a method of modifying an AKT1 protein as disclosed herein. In some embodiments, the method of covalently modifying an AKT1 protein, comprises contacting the AKT1 protein with an exogenous compound, wherein the exogenous compound comprises a reversible electrophilic moiety thereby forming a reversible covalent AKT1 adduct. In some embodiments, the contacting is in vitro or in vivo. In some embodiments, the contacting is in vitro. In some embodiments, the contacting is in vivo. In some embodiments, the AKT1 protein is wild type AKT1 or a mutated AKT1. In some embodiments, the mutated AKT1 is E17K AKT1. In some embodiments, the wild type AKT1 protein is wild type. In some embodiments, the AKT1 protein is E17K AKT1. In some embodiments, the exogenous compound is an AKT1 inhibitor. In some embodiments, the reversible covalent moiety on the AKT1 inhibitor is an aromatic aldehyde. In some embodiments, the reversible covalent AKT1 adduct is formed between the reversible covalent moiety and a lysine reside of the AKT1 protein. In some embodiments, the reversible covalent AKT1 adduct is formed between the aromatic aldehyde and the lysine residue of the AKT1 protein. In some embodiments, reversible covalent AKT1 adduct is formed between the reversible covalent moiety and a lysine residue of the AKT1 protein selected from K17, K40, K49, K158, K163, K179, K276, and K297. In some embodiments, reversible covalent AKT1 adduct is formed between the aromatic aldehyde and a lysine residue of the AKT1 protein selected from K17, K40, K49, K158, K163, K179, K276, and K297. In some embodiments, reversible covalent AKT1 adduct is formed between the aromatic aldehyde and the K17 lysine residue of the AKT1 protein.

In another aspect, the method of covalently modifying an AKT1 protein, comprises contacting the AKT1 protein with an exogenous AKT1 modulator, wherein the AKT1 modulator comprises a reversible electrophilic moiety thereby forming a reversible covalent AKT1 adduct. In some embodiments, the contacting is in vitro or in vivo. In some embodiments, the contacting is in vitro. In some embodiments, the contacting is in vivo. In some embodiments, the AKT1 protein is wild type AKT1 or a mutated AKT1. In some embodiments, the mutated AKT1 is selected from E17K AKT1, E40K AKT1, and E49K AKT1. In some embodiments, the mutated AKT1 is E17K AKT1. In some embodiments, the wild type AKT1 protein is wild type. In some embodiments, the AKT1 protein is E17K AKT1. In some embodiments, the reversible covalent moiety on the AKT1 modulator is an aromatic aldehyde. In some embodiments, the reversible covalent AKT1 adduct is formed between the reversible covalent moiety and a lysine reside of the AKT1 protein. In some embodiments, the reversible covalent AKT1 adduct is formed between the aromatic aldehyde and the lysine residue of the AKT1 protein. In some embodiments, reversible covalent AKT1 adduct is formed between the reversible covalent moiety and a lysine residue of the AKT1 protein selected from K17, K40, K49, K158, K163, K179, K276, and K297. In some embodiments, reversible covalent AKT1 adduct is formed between the aromatic aldehyde and a lysine residue of the AKT1 protein selected from K17, K40, K49, K158, K163, K179, K276, and K297. In some embodiments, reversible covalent AKT1 adduct is formed between the aromatic aldehyde and the K17 lysine residue of the AKT1 protein. In some embodiments, the exogenous AKT1 modulator is an AKT1 inhibitor.

In certain aspects the present disclosure provides a method of attenuating AKT1 activity In some embodiments, the method of covalently modifying an AKT1 protein, comprises contacting the AKT1 protein with an exogenous AKT1 inhibitor, wherein the AKT1 modulator comprises a reversible electrophilic moiety thereby forming a reversible covalent AKT1 adduct. In some embodiments, the contacting is in vitro or in vivo. In some embodiments, the contacting is in vitro. In some embodiments, the contacting is in vivo. In some embodiments, the AKT1 protein is wild type AKT1 or a mutated AKT1. In some embodiments, the mutated AKT1 is selected from E17K AKT1, E40K AKT1, and E49K AKT1. In some embodiments, the mutated AKT1 is E17K AKT1. In some embodiments, the wild type AKT1 protein is wild type. In some embodiments, the AKT1 protein is E17K AKT1. In some embodiments, the reversible covalent moiety on the AKT1 inhibitor is an aromatic aldehyde. In some embodiments, the reversible covalent AKT1 adduct is formed between the reversible covalent moiety and a lysine reside of the AKT1 protein. In some embodiments, the reversible covalent AKT1 adduct is formed between the aromatic aldehyde and the lysine residue of the AKT1 protein. In some embodiments, reversible covalent AKT1 adduct is formed between the reversible covalent moiety and a lysine residue of the AKT1 protein selected from K17, K40, K49, K158, K163, K179, K276, and K297. In some embodiments, reversible covalent AKT1 adduct is formed between the aromatic aldehyde and a lysine residue of the AKT1 protein selected from K17, K40, K49, K158, K163, K179, K276, and K297. In some embodiments, reversible covalent AKT1 adduct is formed between the aromatic aldehyde and the K17 lysine residue of the AKT1 protein.

In some aspects, the method of attenuating AKT1 activity, comprises contacting AKT1 protein with an exogenous compound, wherein the exogenous compound comprises a reversible electrophilic moiety. In some embodiments, the AKT1 protein is wild type AKT1 or a mutated AKT1. In some embodiments, the mutated AKT1 is selected from E17K AKT1, E40K AKT1, and E49K AKT1. In some embodiments, the mutated AKT1 is E17K AKT1. In some embodiments, the wild type AKT1 protein is wild type. In some embodiments, the contacting is in vitro or in vivo. In some embodiments, the contacting is in vitro.

In some embodiments, following the contacting, the AKT1 activity is attenuated by 50% to 95% relative to a control in the absence of the exogenous compound. In some embodiments, following the contacting, the AKT1 activity is attenuated by 75% to 95% relative to a control in the absence of the exogenous compound. In some embodiments, following the contacting, the AKT1 activity is attenuated by 50%, 55%, 60%, 65%, 70%, 75%, 80%, 85%, 90%, 95%, or more relative to a control in the absence of the exogenous compound. In some embodiments, following the contacting, the AKT1 activity is attenuated by 50% or more relative to a control in the absence of the exogenous compound. In some embodiments, following the contacting, the AKT1 activity is attenuated by 70% or more relative to a control in the absence of the exogenous compound.

In some embodiments, following the contacting, the AKT1 activity is attenuated by about 50% to about 95% relative to a control in the absence of the exogenous compound. In some embodiments, following the contacting, the AKT1 activity is attenuated by about 75% to about 95% relative to a control in the absence of the exogenous compound. In some embodiments, following the contacting, the AKT1 activity is attenuated by about 50%, about 55%, about 60%, about 65%, about 70%, about 75%, about 80%, about 85%, about 90%, about 95%, or more relative to a control in the absence of the exogenous compound. In some embodiments, following the contacting, the AKT1 activity is attenuated by about 50% or more relative to a control in the absence of the exogenous compound. In some embodiments, following the contacting, the AKT1 activity is attenuated by about 70% or more relative to a control in the absence of the exogenous compound.

In some embodiments, following the contacting, the AKT1 activity is attenuated by at least 50% to at least 95% relative to a control in the absence of the exogenous compound. In some embodiments, following the contacting, the AKT1 activity is attenuated by at least 75% to at least 95% relative to a control in the absence of the exogenous compound. In some embodiments, following the contacting, the AKT1 activity is attenuated by at least 50%, at least 55%, at least 60%, at least 65%, at least 70%, at least 75%, at least 80%, at least 85%, at least 90%, or at least 95% relative to a control in the absence of the exogenous compound. In some embodiments, following the contacting, the AKT1 activity is attenuated by at least 50% relative to a control in the absence of the exogenous compound. In some embodiments, following the contacting, the AKT1 activity is attenuated by at least 70% relative to a control in the absence of the exogenous compound.

In some embodiments, following the contacting, the AKT1 activity is attenuated by at most 50% to at most 95% relative to a control in the absence of the exogenous compound. In some embodiments, following the contacting, the AKT1 activity is attenuated by at most 75% to at most 95% relative to a control in the absence of the exogenous compound. In some embodiments, following the contacting, the AKT1 activity is attenuated by at most 50% relative to a control in the absence of the exogenous compound. In some embodiments, following the contacting, the AKT1 activity is attenuated by at most 70% relative to a control in the absence of the exogenous compound. In some embodiments, following the contacting, the AKT1 activity is attenuated by at most 50%, at most 55%, at most 60%, at most 65%, at most 70%, at most 75%, at most 80%, at most 85%, at most 90%, or at most 95% relative to a control in the absence of the exogenous compound.

In some embodiments, the exogenous compound is more selective toward mutated AKT1 than wild-type AKT1. In some embodiments, the mutated AKT1 is E17K AKT1. In some embodiments, the exogenous compound is 2-fold to 100-fold more selective for E17K AKT1 over wild-type AKT1. In some embodiments, the exogenous compound is 2-fold to 10-fold more selective for E17K AKT1 over wild-type AKT1. In some embodiments, the exogenous compound is 2-fold to 5-fold more selective for E17K AKT1 over wild-type AKT1. In some embodiments, the exogenous compound is 2-fold more selective for E17K AKT1 over wild-type AKT1. In some embodiments, the exogenous compound is 3-fold more selective for E17K AKT1 over wild-type AKT1. In some embodiments, the exogenous compound is 4-fold more selective for E17K AKT1 over wild-type AKT1. In some embodiments, the exogenous compound is 5-fold more selective for E17K AKT1 over wild-type AKT1. In some embodiments, the exogenous compound is 10-fold more selective for E17K AKT1 over wild-type AKT1. In some embodiments, the exogenous compound is 2-fold, 3-fold, 4-fold, 5-fold, 6-fold, 7-fold, 8-fold, 9-fold, 10-fold, 15-fold, 20-fold, 25-fold, 50-fold, 75-fold, or 100-fold more selective for E17K AKT1 over wild-type AKT1.

In some embodiments, the exogenous compound is at least 2-fold more selective for E17K AKT1 over wild-type AKT1. In some embodiments, the exogenous compound is at least 3-fold more selective for E17K AKT1 over wild-type AKT1. In some embodiments, the exogenous compound is at least 4-fold more selective for E17K AKT1 over wild-type AKT1. In some embodiments, the exogenous compound is at least 5-fold more selective for E17K AKT1 over wild-type AKT1. In some embodiments, the exogenous compound is at least 10-fold more selective for E17K AKT1 over wild-type AKT1. In some embodiments, the exogenous compound is at least 2-fold more selective for E17K AKT1 over wild-type AKT1. In some embodiments, the exogenous compound is at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 50-fold, at least 75-fold, or at least 100-fold more selective for E17K AKT1 over wild-type AKT1.

In some embodiments, the exogenous compound is about 2-fold more selective for E17K AKT1 over wild-type AKT1. In some embodiments, the exogenous compound is about 3-fold more selective for E17K AKT1 over wild-type AKT1. In some embodiments, the exogenous compound is about 4-fold more selective for E17K AKT1 over wild-type AKT1. In some embodiments, the exogenous compound is about 5-fold more selective for E17K AKT1 over wild-type AKT1. In some embodiments, the exogenous compound is about 10-fold more selective for E17K AKT1 over wild-type AKT1. In some embodiments, the exogenous compound is about 2-fold, about 3-fold, about 4-fold, about 5-fold, about 6-fold, about 7-fold, about 8-fold, about 9-fold, about 10-fold, about 15-fold, about 20-fold, about 25-fold, about 50-fold, about 75-fold, or about 100-fold more selective for E17K AKT1 over wild-type AKT1.

In some embodiments, the exogenous compound is at most 2-fold more selective for E17K AKT1 over wild-type AKT1. In some embodiments, the exogenous compound is at most 3-fold more selective for E17K AKT1 over wild-type AKT1. In some embodiments, the exogenous compound is at most 4-fold more selective for E17K AKT1 over wild-type AKT1. In some embodiments, the exogenous compound is at most 5-fold more selective for E17K AKT1 over wild-type AKT1. In some embodiments, the exogenous compound is at most 10-fold more selective for E17K AKT1 over wild-type AKT1. In some embodiments, the exogenous compound is at most 2-fold more selective for E17K AKT1 over wild-type AKT1. In some embodiments, the exogenous compound is at most 2-fold, at most 3-fold, at most 4-fold, at most 5-fold, at most 6-fold, at most 7-fold, at most 8-fold at most 9-fold, at most 10-fold, at most 15-fold, at most 20-fold, at most 25-fold, at most 50-fold at most 75-fold, or at most 100-fold more selective for E17K AKT1 over wild-type AKT1.

In another aspect, the present disclosure provides a method of attenuating AKT1 activity, comprising contacting AKT1 protein with an AKT1 inhibitor, wherein the AKT1 inhibitor comprises a reversible electrophilic moiety. In some embodiments, the AKT1 protein is wild type AKT1 or a mutated AKT1. In some embodiments, the mutated AKT1 is selected from E17K AKT1, E40K AKT1, and E49K AKT1. In some embodiments, the mutated AKT1 is E17K AKT1. In some embodiments, the wild type AKT1 protein is wild type. In some embodiments, the contacting is in vitro or in vivo. In some embodiments, the contacting is in vitro.

In some embodiments, following the contacting, the AKT1 activity is attenuated by 50% to 95% relative to a control in the absence of the exogenous AKT1 inhibitor. In some embodiments, following the contacting, the AKT1 activity is attenuated by 75% to 95% relative to a control in the absence of the exogenous AKT1 inhibitor. In some embodiments, following the contacting, the AKT1 activity is attenuated by 50%, 55%, 60%, 65%, 70%, 75%, 80%, 85%, 90%, 95%, or more relative to a control in the absence of the exogenous AKT1 inhibitor. In some embodiments, following the contacting, the AKT1 activity is attenuated by 50% or more relative to a control in the absence of the exogenous AKT1 inhibitor. In some embodiments, following the contacting, the AKT1 activity is attenuated by 70% or more relative to a control in the absence of the exogenous AKT1 inhibitor.

In some embodiments, following the contacting, the AKT1 activity is attenuated by about 50% to about 95% relative to a control in the absence of the exogenous AKT1 inhibitor. In some embodiments, following the contacting, the AKT1 activity is attenuated by about 75% to about 95% relative to a control in the absence of the exogenous AKT1 inhibitor. In some embodiments, following the contacting, the AKT1 activity is attenuated by about 50%, about 55%, about 60%, about 65%, about 70%, about 75%, about 80%, about 85%, about 90%, about 95%, or more relative to a control in the absence of the exogenous AKT1 inhibitor. In some embodiments, following the contacting, the AKT1 activity is attenuated by about 50% or more relative to a control in the absence of the exogenous AKT1 inhibitor. In some embodiments, following the contacting, the AKT1 activity is attenuated by about 70% or more relative to a control in the absence of the exogenous AKT1 inhibitor.

In some embodiments, following the contacting, the AKT1 activity is attenuated by at least 50% to at least 95% relative to a control in the absence of the exogenous AKT1 inhibitor. In some embodiments, following the contacting, the AKT1 activity is attenuated by at least 75% to at least 95% relative to a control in the absence of the exogenous AKT1 inhibitor. In some embodiments, following the contacting, the AKT1 activity is attenuated by at least 50%, at least 55%, at least 60%, at least 65%, at least 70%, at least 75%, at least 80%, at least 85%, at least 90%, or at least 95% relative to a control in the absence of the exogenous AKT1 inhibitor. In some embodiments, following the contacting, the AKT1 activity is attenuated by at least 50% relative to a control in the absence of the exogenous AKT1 inhibitor. In some embodiments, following the contacting, the AKT1 activity is attenuated by at least 70% relative to a control in the absence of the exogenous AKT1 inhibitor.

In some embodiments, following the contacting, the AKT1 activity is attenuated by at most 50% to at most 95% relative to a control in the absence of the exogenous AKT1 inhibitor. In some embodiments, following the contacting, the AKT1 activity is attenuated by at most 75% to at most 95% relative to a control in the absence of the exogenous AKT1 inhibitor. In some embodiments, following the contacting, the AKT1 activity is attenuated by at most 50% relative to a control in the absence of the exogenous AKT1 inhibitor. In some embodiments, following the contacting, the AKT1 activity is attenuated by at most 70% relative to a control in the absence of the exogenous AKT1 inhibitor. In some embodiments, following the contacting, the AKT1 activity is attenuated by at most 50%, at most 55%, at most 60%, at most 65%, at most 70%, at most 75%, at most 80%, at most 85%, at most 90%, or at most 95% relative to a control in the absence of the exogenous AKT1 inhibitor.

### COMPOUNDS

A compound of Formula (I):

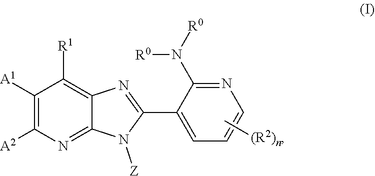

or a pharmaceutically acceptable salt thereof; wherein,
   R⁰ is independently selected at each occurrence from hydrogen, alkyl, heteroalkyl, alkenyl, alkynyl, any of which is independently unsubstituted or substituted;
   R¹ and R² are each independently selected from hydrogen, halogen, alkyl, heteroalkyl, alkenyl, alkynyl, —OR¹⁰, —SR¹⁰, —N(R¹⁰)₂, —NO₂, and —CN, wherein each alkyl, heteroalkyl, alkenyl, and alkynyl of R¹ and R² is independently unsubstituted or substituted;
   n is selected from 0, 1, 2, and 3;
   A¹ and A² are each independently selected from: hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, —CN, alkyl, heteroalkyl, alkenyl, alkynyl, carbocycle and heterocycle, wherein each alkyl, heteroalkyl, alkenyl, alkynyl, carbocycle and heterocycle of A¹ and A² is independently unsubstituted or substituted;

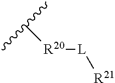

Z is represented by R²¹ wherein,
   R²⁰ is selected from heterocyclene and phenylene, any of which is unsubstituted or substituted;
   L is a bond or represented by -L¹-L²-L³-L⁴-, wherein each L¹, L², L³, and L⁴ is independently selected from (a) and (b):
      (a) —O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, —(R¹⁴)NC(O)N(R¹⁴)—, and —(R¹⁴)NC(O)N(R¹⁴)N(R¹⁴)—; and
      (b) alkylene, heteroalkylene, alkenylene, alkynylene, carbocyclene, and heterocyclene, any of which is independently unsubstituted or substituted;
      wherein L², L³, and L⁴ are each optionally absent;
      wherein no more than two of L¹, L², L³, and L⁴ are selected from (a) and the two selected are not adjacent to each other;
   R²¹ is selected from heterocycle and carbocycle, any of which is substituted with C(O)H, wherein R²¹ is optionally further substituted; and
   R¹⁰, R¹¹, and R¹⁴ are each independently selected at each occurrence from hydrogen, alkyl, heteroalkyl, carbocycle, and heterocycle, any of which is independently unsubstituted or substituted.

In some embodiments, for the compound or salt of Formula (I), R⁰ is independently selected at each occurrence from hydrogen, alkyl, heteroalkyl, alkenyl, and alkynyl, any of which is independently unsubstituted or substituted. In some embodiments, R⁰ is independently selected at each occurrence from hydrogen, alkyl, and heteroalkyl, any of which is independently unsubstituted or substituted. R⁰ is hydrogen.

In some embodiments, for the compound or salt of Formula (I), R¹ is selected from hydrogen, halogen, alkyl, heteroalkyl, alkenyl, alkynyl, —OR¹⁰, —SR¹⁰, —N(R¹⁰)₂, —NO₂, and —CN, wherein each alkyl, heteroalkyl, alkenyl, and alkynyl is independently unsubstituted or substituted. In some embodiments, R¹ is selected from hydrogen, halogen, alkyl, heteroalkyl, alkenyl, and alkynyl, wherein each alkyl, wherein each alkyl, heteroalkyl, alkenyl, and alkynyl is independently unsubstituted or substituted. In some embodiments, R¹ is selected from hydrogen, halogen, alkyl, and heteroalkyl, wherein each alkyl and heteroalkyl is independently unsubstituted or substituted In some embodiments, R¹ is selected from hydrogen, halogen, alkyl, heteroalkyl, —OR¹⁰, —SR¹⁰, —N(R¹⁰)₂, —NO₂, and —CN, wherein each alkyl, and heteroalkyl is independently unsubstituted or substituted; and wherein R¹⁰ is independently selected at each occurrence from hydrogen, unsubstituted alkyl, and substituted alkyl. In some embodiments, R¹ is selected from hydrogen, halogen, unsubstituted alkyl, —OR¹⁰, —SR¹⁰, —N(R¹⁰)₂, —NO₂, and —CN, and wherein R¹⁰ is independently selected at each occurrence from hydrogen, unsubstituted alkyl, and substituted alkyl. In some embodiments, R¹ is selected from hydrogen, halogen, and unsubstituted alkyl. In some embodiments, R¹ is hydrogen.

In some embodiments, for the compound or salt of Formula (I), R² is selected from hydrogen, halogen, alkyl, heteroalkyl, alkenyl, alkynyl, —OR¹⁰, —SR¹⁰, —N(R¹⁰)₂, —NO₂, and —CN, wherein each alkyl, heteroalkyl, alkenyl, and alkynyl is independently unsubstituted or substituted. In some embodiments, R² is selected from hydrogen, halogen, alkyl, heteroalkyl, alkenyl, and alkynyl, wherein each alkyl, wherein each alkyl, heteroalkyl, alkenyl, and alkynyl is independently unsubstituted or substituted. In some embodiments, R² is selected from hydrogen, halogen, alkyl, and heteroalkyl, wherein each alkyl and heteroalkyl is independently unsubstituted or substituted In some embodiments, R² is selected from hydrogen, halogen, alkyl, heteroalkyl, —OR¹⁰, —SR¹⁰, —N(R¹⁰)₂, —NO₂, and —CN, wherein each alkyl, and heteroalkyl is independently unsubstituted or substituted; and wherein R¹⁰ is independently selected at each occurrence from hydrogen, unsubstituted alkyl, and substituted alkyl. In some embodiments, R² is selected from hydrogen, halogen, unsubstituted alkyl, —OR¹⁰, —SR¹⁰, —N(R¹⁰)₂, —NO₂, and —CN, and wherein R¹⁰ is independently selected at each occurrence from hydrogen, unsubstituted alkyl, and substituted alkyl. In some embodiments, R² is selected from hydrogen, halogen, and unsubstituted alkyl.

In some embodiments, for the compound or salt of Formula (I), n is selected from 0, 1, 2, and 3. In some embodiments, n is selected from 0, 1, and 2. In some embodiments, n is selected from 0 and 1. In some embodiments, n is 0.

In some embodiments, the compound or salt of Formula (I) is a compound represented by the structure of Formula (I-A):

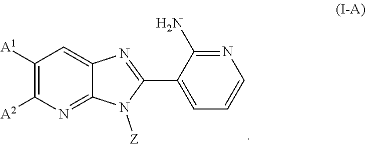

or a pharmaceutically acceptable salt thereof, wherein A¹, A², and Z are each defined as in Formula (I).

In some embodiments, for the compound or salt of Formula (I) or Formula (I-A), A₁ and A² are each independently selected from: hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, —CN, alkyl, heteroalkyl, alkenyl, alkynyl, carbocycle and heterocycle, wherein each alkyl, heteroalkyl, alkenyl, alkynyl, carbocycle and heterocycle of A¹ and A² is independently unsubstituted or substituted. In some embodiments, for the compound or salt of Formula (I) or Formula (I-A), A¹ and A² are each independently selected from: hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, —CN, alkyl, heteroalkyl, alkenyl, alkynyl, carbocycle and heterocycle, wherein each alkyl, heteroalkyl, alkenyl, alkynyl, carbocycle and heterocycle of A¹ and A² is independently unsubstituted or substituted; and R¹¹ is independently selected at each occurrence from hydrogen, alkyl, heteroalkyl, carbocycle, and heterocycle, any of which is independently unsubstituted or substituted. In some embodiments, A¹ and A² are each independently selected from: hydrogen, halogen, —OR¹¹, —N(R¹¹)₂, —C(O)R¹¹, —C(O)N(R¹¹)₂, —N(R¹¹)C(O)R¹¹, alkyl, heteroalkyl, alkenyl, alkynyl, carbocycle and heterocycle, wherein each alkyl, heteroalkyl, alkenyl, alkynyl, carbocycle and heterocycle of A¹ and A² is independently unsubstituted or substituted. In some embodiments, A¹ and A² are each independently selected from: hydrogen, halogen, —OR¹¹, —N(R¹¹)₂, alkyl, heteroalkyl, alkynyl, carbocycle and heterocycle, wherein each alkyl, heteroalkyl, alkynyl, carbocycle and heterocycle of A¹ and A² is independently unsubstituted or substituted. In some embodiments, A¹ and A² are each independently selected from: hydrogen, halogen, —OR¹¹, —N(R¹¹)₂, alkyl, heteroalkyl, alkynyl, carbocycle and heterocycle, wherein each alkyl, heteroalkyl, alkynyl, carbocycle and heterocycle of A¹ and A² is independently unsubstituted or substituted; and R¹¹ is independently selected at each occurrence from hydrogen and unsubstituted alkyl. In some embodiments, A¹ and A² are each independently selected from: alkyl, heteroalkyl, alkenyl, alkynyl, carbocycle and heterocycle, any of which is independently unsubstituted or substituted. In some embodiments, A¹ and A² are each independently selected from: alkyl, heteroalkyl, alkynyl, carbocycle and heterocycle, any of which is independently unsubstituted or substituted. In some embodiments, A¹ and A² are hydrogen.

In some embodiments, for the compound or salt of Formula (I) or Formula (I-A), A¹ and A² are each independently selected from: hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, —CN, alkyl, heteroalkyl, alkenyl, alkynyl, carbocycle and heterocycle, wherein each alkyl, heteroalkyl, alkenyl, alkynyl, carbocycle and heterocycle of A¹ and A² is independently unsubstituted or substituted with one or more substituents selected from
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
alkyl, heteroalkyl, alkenyl, and alkynyl, any one of which is unsubstituted or substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), —CN, unsubstituted carbocycle, substituted carbocycle, unsubstituted heterocycle, and substituted heterocycle; and
carbocycle and heterocycle; any one of which is unsubstituted or substituted with one or more substituents selected from halogen, alkyl, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, unsubstituted carbocycle, substituted carbocycle, unsubstituted heterocycle, and substituted heterocycle.

In some embodiments, for the compound or salt of Formula (I) or Formula (I-A), A¹ and A² are each independently selected from: hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, —CN, alkyl, heteroalkyl, alkenyl, alkynyl, carbocycle and heterocycle, wherein each alkyl, heteroalkyl, alkenyl, alkynyl, carbocycle and heterocycle of A¹ and A² is independently unsubstituted or substituted with one or more substituents selected from
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
alkyl, heteroalkyl, alkenyl, and alkynyl, any one of which is unsubstituted or substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), —CN, unsubstituted carbocycle, substituted carbocycle, unsubstituted heterocycle, and substituted heterocycle; and
carbocycle and heterocycle; any one of which is unsubstituted or substituted with one or more substituents selected from halogen,
   alkyl, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, unsubstituted carbocycle, substituted carbocycle, unsubstituted heterocycle, and substituted heterocycle; and
R¹¹ is independently selected at each occurrence from hydrogen and unsubstituted alkyl.

In some embodiments, for the compound or salt of Formula (I) or Formula (I-A), A¹ and A² are each independently selected from: hydrogen, halogen, —OR¹¹, —N(R¹¹)₂, —C(O)R¹¹, —C(O)N(R¹¹)₂, —N(R¹¹)C(O)R¹¹, alkyl, heteroalkyl, alkenyl, alkynyl, carbocycle and heterocycle, wherein each alkyl, heteroalkyl, alkenyl, alkynyl, carbocycle and heterocycle of A and A² is independently unsubstituted or substituted with one or more substituents selected from
halogen, —OR¹¹, —C(O)R¹¹, —C(O)N(R¹¹)₂, —N(R¹¹)C(O)R¹¹, —N(R¹¹)S(O)₂R¹¹, ═O, and —CN; alkyl, heteroalkyl, alkenyl, and alkynyl, any one of which is unsubstituted or substituted with one or more substituents independently selected from:
   halogen, —OR¹¹, —C(O)R¹¹, —C(O)N(R¹¹)₂, —N(R¹¹)C(O)R¹¹, —N(R¹¹)S(O)₂R¹¹, ═O, —CN, unsubstituted carbocycle, substituted carbocycle, unsubstituted heterocycle, and substituted heterocycle; and
carbocycle and heterocycle; any one of which is unsubstituted or substituted with one or more substituents selected from halogen, alkyl, —OR¹¹, —C(O)R¹¹, —C(O)N(R¹¹)₂, —N(R¹¹)C(O)R¹¹, ═O, —CN, unsubstituted carbocycle, substituted carbocycle, unsubstituted heterocycle, and substituted heterocycle.

In some embodiments, for the compound or salt of Formula (I) or Formula (I-A), A¹ and A² are each independently selected from: hydrogen, halogen, —OR¹¹, —N(R¹¹)₂, alkyl, heteroalkyl, alkynyl, carbocycle and heterocycle, wherein each alkyl, heteroalkyl, alkynyl, carbocycle and heterocycle of A¹ and A² is independently unsubstituted or substituted with one or more substituents selected from
halogen, —OR¹¹, —C(O)R¹¹, —N(R¹¹)C(O)R¹¹, —N(R¹¹)S(O)₂R¹¹, alkyl, and heteroalkyl; and
   carbocycle and heterocycle; any one of which is unsubstituted or substituted with one or more substituents selected from —N(R¹¹)C(O)R¹¹, unsubstituted heterocycle, and substituted heterocycle.

In some embodiments, for the compound or salt of Formula (I) or Formula (I-A), A¹ and A² are each independently selected from: hydrogen, halogen, —OR¹¹, —N(R¹¹)₂, alkyl, heteroalkyl, alkynyl, carbocycle and heterocycle, wherein each alkyl, heteroalkyl, alkynyl, carbocycle and heterocycle of A¹ and A² is independently unsubstituted or substituted with one or more substituents selected from
halogen, —OR¹¹, —C(O)R¹¹, —N(R¹¹)C(O)R¹¹, —N(R¹¹)S(O)₂R¹¹, and unsubstituted alkyl; and
heterocycle unsubstituted or substituted with one or more substituents selected from —N(R¹¹)C(O)R¹¹, unsubstituted heterocycle, and substituted heterocycle.

In some embodiments, for the compound or salt of Formula (I) or Formula (I-A), A¹ and A² are each independently selected from: hydrogen, halogen, —OR¹¹, —N(R¹¹)₂, alkyl, heteroalkyl, alkynyl, carbocycle and heterocycle, wherein each alkyl, heteroalkyl, alkynyl, carbocycle and heterocycle of A¹ and A² is independently unsubstituted or substituted with one or more substituents selected from
halogen, —OR¹¹, —C(O)R¹¹, —N(R¹¹)C(O)R¹¹, —N(R¹¹)S(O)₂R¹¹, and unsubstituted alkyl; and
heterocycle unsubstituted or substituted with one or more substituents selected from —N(R¹¹)C(O)R¹¹, unsubstituted heterocycle, and substituted heterocycle; and
R¹¹ is independently selected at each occurrence from hydrogen and unsubstituted alkyl.

In some embodiments, for the compound or salt of Formula (I) or Formula (I-A), A¹ is selected from: hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, —CN, alkyl, heteroalkyl, alkenyl, alkynyl, carbocycle and heterocycle, wherein each alkyl, heteroalkyl, alkenyl, alkynyl, carbocycle and heterocycle is independently unsubstituted or substituted; and wherein R¹¹ is independently selected at each occurrence from hydrogen and unsubstituted alkyl. In some embodiments, A¹ is selected from: hydrogen, halogen, alkyl, carbocycle and heterocycle, wherein each alkyl, carbocycle and heterocycle is independently unsubstituted or substituted. In some embodiments, A¹ is selected from: hydrogen and phenyl. In some embodiments, A¹ is hydrogen.

In some embodiments, for the compound or salt of Formula (I) or Formula (I-A), A² is selected from: hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, —CN, alkyl, heteroalkyl, alkenyl, alkynyl, carbocycle and heterocycle, wherein each alkyl, heteroalkyl, alkenyl, alkynyl, carbocycle and heterocycle is independently unsubstituted or substituted. In some embodiments, A² is selected from: hydrogen, halogen, —OR¹¹, —N(R¹¹)₂, —C(O)R¹¹, —C(O)N(R¹¹)₂, —N(R¹¹)C(O)R¹¹, alkyl, heteroalkyl, alkenyl, alkynyl, carbocycle and heterocycle, wherein each alkyl, heteroalkyl, alkenyl, alkynyl, carbocycle and heterocycle is independently unsubstituted or substituted. In some embodiments, A² is selected from: hydrogen, halogen, —OR¹¹, —N(R¹¹)₂, alkyl, heteroalkyl, alkynyl, carbocycle and heterocycle, wherein each alkyl, heteroalkyl, alkynyl, carbocycle and heterocycle is independently unsubstituted or substituted. In some embodiments, A² is selected from: hydrogen, halogen, —OR¹¹, —N(R¹¹)₂, alkyl, heteroalkyl, alkynyl, carbocycle and heterocycle, wherein each alkyl, heteroalkyl, alkynyl, carbocycle and heterocycle is independently unsubstituted or substituted; and wherein R¹¹ is independently selected at each occurrence from hydrogen and unsubstituted alkyl. In some embodiments, A² is selected from: alkyl, heteroalkyl, alkenyl, alkynyl, carbocycle and heterocycle, any of which is independently unsubstituted or substituted. In some embodiments, A² is selected from: alkyl, heteroalkyl, alkynyl, carbocycle and heterocycle, any of which is independently unsubstituted or substituted.

In some embodiments, for the compound or salt of Formula (I) or Formula (I-A), A² is selected from: hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, —CN, alkyl, heteroalkyl, alkenyl, alkynyl, carbocycle and heterocycle, wherein each alkyl, heteroalkyl, alkenyl, alkynyl, carbocycle and heterocycle is independently unsubstituted or substituted with one or more substituents selected from
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
alkyl, heteroalkyl, alkenyl, and alkynyl, any one of which is unsubstituted or substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), —CN, unsubstituted carbocycle, substituted carbocycle, unsubstituted heterocycle, and substituted heterocycle; and
   carbocycle and heterocycle; any one of which is unsubstituted or substituted with one or more substituents selected from halogen, alkyl, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, unsubstituted carbocycle, substituted carbocycle, unsubstituted heterocycle, and substituted heterocycle.

In some embodiments, for the compound or salt of Formula (I) or Formula (I-A), A² is selected from: hydrogen, halogen, —OR¹¹, —SR¹¹, —N(R¹¹)₂, —C(O)R¹¹, —C(O)N(R¹¹)₂,

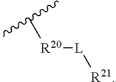

In some embodiments, for the compound or salt of Formula (I) or Formula (I-A), R²⁰ is selected from heterocyclene and phenylene, any of which is unsubstituted or substituted. In some embodiments, R²⁰ is selected from unsubstituted heterocyclene and unsubstituted phenylene. In some embodiments, R²⁰ is selected from 5- to 6-membered heterocyclene and phenylene, any of which are unsubstituted or substituted. In some embodiments, R²⁰ is selected from unsubstituted 5- to 6-membered heterocyclene and unsubstituted phenylene. In some embodiments, R²⁰ is selected from 6-membered heteroarylene and phenylene, any of which are unsubstituted or substituted. In some embodiments, R²⁰ is selected from unsubstituted 6-membered heteroarylene and unsubstituted phenylene. In some embodiments, R²⁰ is selected from pyridinylene and phenylene, any one of which are substituted or unsubstituted. In some embodiments, R²⁰ is selected from unsubstituted pyridinylene and unsubstituted phenylene. In some embodiments, R²⁰ is unsubstituted phenylene.

In some embodiments, for the compound or salt of Formula (I) or Formula (I-A), L is a bond or represented by -L¹-L²-L³-L⁴-.

In some embodiments, for the compound or salt of Formula (I) or Formula (I-A), L¹, L², L³, and L⁴ are each independently selected from (a) and (b):
(a) —O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, —(R¹⁴)NC(O)N(R¹⁴)—, and —(R¹⁴)NC(O)N(R¹⁴)N(R¹⁴)—; and
(b) alkylene, heteroalkylene, alkenylene, alkynylene, carbocyclene, and heterocyclene, any of which is independently unsubstituted or substituted;
wherein L², L³, and L⁴ are each optionally absent;
wherein no more than two of L¹, L², L³, and L⁴ are selected from (a) and the two selected are not adjacent to each other.

In some embodiments, for the compound or salt of Formula (I) or Formula (I-A), L¹, L², L³, and L⁴ are each independently selected from (a) and (b):
(a) —O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, —(R¹⁴)NC(O)N(R¹⁴)—, and —(R¹⁴)NC(O)N(R¹⁴)N(R¹⁴)—; and
(b) alkylene, heteroalkylene, alkenylene, alkynylene, carbocyclene, and heterocyclene, any of which is independently unsubstituted or substituted;
wherein L², L³, and L⁴ are each optionally absent;
wherein no more than two of L¹, L², L³, and L⁴ are selected from (a) and the two selected are not adjacent to each other; and
R¹⁴ is independently selected at each occurrence from hydrogen, alkyl, heteroalkyl, carbocycle, and heterocycle, any of which is independently unsubstituted or substituted.

In some embodiments, for the compound or salt of Formula (I) or Formula (I-A), L¹, L², L³, and L⁴ are each independently selected from (a) and (b):
(a) —O—, —S—, —S(O)—, —S(O)₂—, —N(R¹⁴)—, —N(R¹⁴)C(O)—, —N(R¹⁴)C(O)O—, and —N(R¹⁴)S(O)₂—; and
(b) alkylene, heteroalkylene, alkenylene, alkynylene, carbocyclene, and heterocyclene, any of which is independently unsubstituted or substituted;
wherein L², L³, and L⁴ are each optionally absent;
wherein no more than two of L¹, L², L³, and L⁴ are selected from (a) and the two selected are not adjacent to each other.

In some embodiments, for the compound or salt of Formula (I) or Formula (I-A), L¹, L², L³, and L⁴ are each independently selected from (a) and (b):
(a) —N(R¹⁴)—, —N(R¹⁴)C(O)—, and —N(R¹⁴)S(O)₂—; and
(b) alkylene, heteroalkylene, alkynylene, carbocyclene, and heterocyclene, any of which is independently unsubstituted or substituted;
wherein L², L³, and L⁴ are each optionally absent;
wherein no more than two of L¹, L², L³, and L⁴ are selected from (a) and the two selected are not adjacent to each other.

In some embodiments, for the compound or salt of Formula (I) or Formula (I-A), L¹, L², L³, and L⁴ are each independently selected from (a) and (b):
(a) —N(R¹⁴)—, —N(R¹⁴)C(O)—, and —N(R¹⁴)S(O)₂—; and
(b) alkylene, heteroalkylene, alkynylene, carbocyclene, and heterocyclene, any of which is independently unsubstituted or substituted;
wherein L², L³, and L⁴ are each optionally absent;
wherein no more than two of L¹, L², L³, and L⁴ are selected from (a) and the two selected are not adjacent to each other; and
R¹⁴ is independently selected at each occurrence from hydrogen, unsubstituted alkyl, and substituted alkyl.

In some embodiments, for the compound or salt of Formula (I) or Formula (I-A), L², L³, and L⁴ are absent; and L¹ is selected from
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, —(R¹⁴)NC(O)N(R¹⁴)—, and —(R¹⁴)NC(O)N(R¹⁴)N(R¹⁴)—; and
alkylene, heteroalkylene, alkenylene, alkynylene, carbocyclene, and heterocyclene, any of which is independently unsubstituted or substituted.

In some embodiments, for the compound or salt of Formula (I) or Formula (I-A), L², L³, and L⁴ are absent; and L¹ is selected from
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, —(R¹⁴)NC(O)N(R¹⁴)—, and —(R¹⁴)NC(O)N(R¹⁴)N(R¹⁴)—; and
alkylene, heteroalkylene, alkenylene, alkynylene, carbocyclene, and heterocyclene, any of which is independently unsubstituted or substituted; and
R¹⁴ is independently selected at each occurrence from hydrogen, alkyl, heteroalkyl, carbocycle, and heterocycle, any of which is independently unsubstituted or substituted.

In some embodiments, for the compound or salt of Formula (I) or Formula (I-A), L², L³, and L⁴ are absent; and L¹ is selected from
—N(R¹⁴)—, —N(R¹⁴)C(O)—, and —N(R¹⁴)S(O)₂—; and
alkylene, heteroalkylene, alkynylene, carbocyclene, and heterocyclene, any of which is independently unsubstituted or substituted.

In some embodiments, for the compound or salt of Formula (I) or Formula (I-A), L², L³, and L⁴ are absent; and L¹ is selected from
—N(R¹⁴)—, —N(R¹⁴)C(O)—, and —N(R¹⁴)S(O)₂—; and
alkylene, heteroalkylene, alkynylene, carbocyclene, and heterocyclene, any of which is independently unsubstituted or substituted; and
R¹⁴ is independently selected at each occurrence from hydrogen, unsubstituted alkyl, and substituted alkyl.

In some embodiments, for the compound or salt of Formula (I) or Formula (I-A), L², L³, and L⁴ are absent; and L¹ is selected from alkylene, heteroalkylene, alkynylene, carbocyclene, and heterocyclene, any of which is independently unsubstituted or substituted.

In some embodiments, for the compound or salt of Formula (I) or Formula (I-A), L², L³, and L⁴ are absent; and L¹ is selected from unsubstituted alkylene, substituted alkylene, unsubstituted heteroalkylene, and substituted heteroalkylene.

In some embodiments, for the compound or salt of Formula (I) or Formula (I-A), L³ and L⁴ are absent; and L¹ and L² are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, —(R¹⁴)NC(O)N(R¹⁴)—, and —(R¹⁴)NC(O)N(R¹⁴)N(R¹⁴)—; and
alkylene, heteroalkylene, alkenylene, alkynylene, carbocyclene, and heterocyclene, any of which is independently unsubstituted or substituted.

In some embodiments, for the compound or salt of Formula (I) or Formula (I-A), L³ and L⁴ are absent; and L¹ and L² are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, —(R¹⁴)NC(O)N(R¹⁴)—, and —(R¹⁴)NC(O)N(R¹⁴)N(R¹⁴)—; and
alkylene, heteroalkylene, alkenylene, alkynylene, carbocyclene, and heterocyclene, any of which is independently unsubstituted or substituted; and
R¹⁴ is independently selected at each occurrence from hydrogen, alkyl, heteroalkyl, carbocycle, and heterocycle, any of which is independently unsubstituted or substituted.

In some embodiments, for the compound or salt of Formula (I) or Formula (I-A), L³ and L⁴ are absent; and L¹ and L² are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—; and
alkylene, heteroalkylene, alkenylene, alkynylene, carbocyclene, and heterocyclene, any one of which is independently unsubstituted or substituted.

In some embodiments, for the compound or salt of Formula (I) or Formula (I-A), L³ and L⁴ are absent; and L¹ and L² are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—; and
alkylene, heteroalkylene, alkenylene, alkynylene, carbocyclene, and heterocyclene, any one of which is independently unsubstituted or substituted; and
R¹⁴ is independently selected at each occurrence from hydrogen, unsubstituted alkyl, and substituted alkyl.

In some embodiments, for the compound or salt of Formula (I) or Formula (I-A), L³ and L⁴ are absent; and L¹ and L² are independently selected from:
—N(R¹⁴)—, —N(R¹⁴)C(O)—, and —N(R¹⁴)S(O)₂—; and
alkylene, heteroalkylene, alkynylene, carbocyclene, and heterocyclene, any of which is independently unsubstituted or substituted.

In some embodiments, for the compound or salt of Formula (I) or Formula (I-A), L³ and L⁴ are absent; and L¹ and L² are independently selected from:
—N(R¹⁴)—, —N(R¹⁴)C(O)—, and —N(R¹⁴)S(O)₂—; and
alkylene, heteroalkylene, alkynylene, carbocyclene, and heterocyclene, any of which is independently unsubstituted or substituted; and
R¹⁴ is independently selected at each occurrence from hydrogen, unsubstituted alkyl, and substituted alkyl.

In some embodiments, for the compound or salt of Formula (I) or Formula (I-A), L⁴ is absent; and each of L¹, L², and L³ are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, —(R¹⁴)NC(O)N(R¹⁴)—, and —(R¹⁴)NC(O)N(R⁴)N(R¹⁴)—; and
alkylene, heteroalkylene, alkenylene, alkynylene, carbocyclene, and heterocyclene, any of which is independently unsubstituted or substituted.

In some embodiments, for the compound or salt of Formula (I) or Formula (I-A), L⁴ is absent; and each of L¹, L², and L³ are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, —(R¹⁴)NC(O)N(R¹⁴)—, and —(R¹⁴)NC(O)N(R⁴)N(R¹⁴)—; and
alkylene, heteroalkylene, alkenylene, alkynylene, carbocyclene, and heterocyclene, any of which is independently unsubstituted or substituted; and
R¹⁴ is independently selected at each occurrence from hydrogen, alkyl, heteroalkyl, carbocycle, and heterocycle, any of which is independently unsubstituted or substituted.

In some embodiments, for the compound or salt of Formula (I) or Formula (I-A), L⁴ is absent; and each of L¹, L², and L³ are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—; and
alkylene, heteroalkylene, alkenylene, alkynylene, carbocyclene, and heterocyclene, any one of which is independently unsubstituted or substituted.

In some embodiments, for the compound or salt of Formula (I) or Formula (I-A), L⁴ is absent; and each of L¹, L², and L³ are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—; and
alkylene, heteroalkylene, alkenylene, alkynylene, carbocyclene, and heterocyclene, any one of which is independently unsubstituted or substituted; and
R¹⁴ is independently selected at each occurrence from hydrogen, unsubstituted alkyl, and substituted alkyl.

In some embodiments, for the compound or salt of Formula (I) or Formula (I-A), L⁴ is absent; and each of L¹, L², and L³ are independently selected from:
—N(R¹⁴)—, —N(R¹⁴)C(O)—, and —N(R¹⁴)S(O)₂—; and
alkylene, heteroalkylene, alkynylene, carbocyclene, and heterocyclene, any of which is independently unsubstituted or substituted.

In some embodiments, for the compound or salt of Formula (I) or Formula (I-A), L⁴ is absent; and each of L¹, L², and L³ are independently selected from:
—N(R¹⁴)—, —N(R¹⁴)C(O)—, and —N(R¹⁴)S(O)₂—; and
alkylene, heteroalkylene, alkynylene, carbocyclene, and heterocyclene, any of which is independently unsubstituted or substituted; and
R¹⁴ is independently selected at each occurrence from hydrogen, unsubstituted alkyl, and substituted alkyl.

In some embodiments, for the compound or salt of Formula (I) or Formula (I-A), R²¹ is selected from heterocycle and carbocycle, any of which is substituted with C(O)H, wherein R²¹ is optionally further substituted. In some embodiments, R²¹ is selected from 5- to 6-membered heterocycle and C₃₋₆ carbocycle, any of which is substituted with C(O)H, wherein R²¹ is optionally further substituted. In some embodiments, R²¹ is selected from 6-membered heteroaryl and phenyl, any of which is substituted with C(O)H, wherein R²¹ is optionally further substituted. In some embodiments, R²¹ is selected from pyridinyl and phenyl, any of which is substituted with C(O)H, wherein R²¹ is optionally further substituted. In some embodiments, R²¹ is phenyl substituted with C(O)H and is optionally further substituted.

In some embodiments, for the compound or salt of Formula (I) or Formula (I-A), R²¹ is selected from heterocycle and carbocycle, any of which is substituted with C(O)H, wherein R²¹ is further optionally substituted with one or more substituents independently selected from: halogen, unsubstituted alkyl, unsubstituted
haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN. In some embodiments, R²¹ is selected from heterocycle and carbocycle, any of which is substituted with C(O)H, wherein R²¹ is further optionally substituted with one or more substituents independently selected from: halogen, unsubstituted alkyl, unsubstituted haloalkyl, —OR⁵, —SR¹¹, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN; and wherein R¹⁵ is independently selected at each occurrence from hydrogen, unsubstituted alkyl, unsubstituted carbocycle, unsubstituted heterocycle, and substituted heterocycle. In some embodiments, R²¹ is selected from heterocycle and carbocycle, any of which is substituted with C(O)H, wherein R²¹ is further optionally substituted with one or more substituents independently selected from: halogen, unsubstituted alkyl, unsubstituted haloalkyl, —OR¹⁵, —C(O)R¹⁵, —C(O)N(R¹⁵)₂, —C(O)OR¹⁵, —OC(O)R¹⁵, —N(R¹⁵)C(O)R¹⁵, —N(R¹⁵)C(O)N(R¹⁵)₂, ═O, and —CN. In some embodiments, R²¹ is selected from heterocycle and carbocycle, any of which is substituted with C(O)H, wherein R²¹ is further optionally substituted with one or more substituents independently selected from: halogen, unsubstituted alkyl, unsubstituted haloalkyl, —OR¹⁵, —N(R¹⁵)C(O)R¹⁵, and —N(R¹⁵)C(O)N(R¹⁵)₂. In some embodiments, R²¹ is selected from heterocycle and carbocycle, any of which is substituted with C(O)H, wherein R²¹ is further optionally substituted with one or more substituents independently selected from: halogen, unsubstituted alkyl, unsubstituted haloalkyl, —OR¹⁵, —N(R¹⁵)C(O)R¹⁵, and —N(R¹⁵)C(O)N(R¹⁵)₂; and R¹⁵ is independently selected at each occurrence from hydrogen, unsubstituted alkyl, unsubstituted carbocycle, unsubstituted heterocycle, and substituted heterocycle.

In some embodiments, for the compound or salt of Formula (I) or Formula (I-A), R²¹ is selected from 5- to 6-membered heterocycle and C₃₋₆ carbocycle, any of which is substituted with C(O)H, wherein R²¹ is further optionally substituted with one or more substituents independently selected from: halogen, unsubstituted alkyl, unsubstituted haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN. In some embodiments, R²¹ is selected from 5- to 6-membered heterocycle and C₃₋₆ carbocycle, any of which is substituted with C(O)H, wherein R²¹ is further optionally substituted with one or more substituents independently selected from: halogen, unsubstituted alkyl, unsubstituted haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O) ₂R¹⁵, —NO₂, ═O, ═S, ═N(R¹⁵), and —CN; and wherein R¹⁵ is independently selected at each occurrence from hydrogen, unsubstituted alkyl, unsubstituted carbocycle, unsubstituted heterocycle, and substituted heterocycle. In some embodiments, R²¹ is selected from 5- to 6-membered heterocycle and C₃₋₆ carbocycle, any of which is substituted with C(O)H, wherein R²¹ is further optionally substituted with one or more substituents independently selected from: halogen, unsubstituted alkyl, unsubstituted haloalkyl, —OR¹⁵, —C(O)R¹⁵, —C(O)N(R¹⁵)₂, —C(O)OR¹⁵, —OC(O)R¹⁵, —N(R¹⁵)C(O)R¹⁵, —N(R¹⁵)C(O)N(R¹⁵)₂, ═O, and —CN. In some embodiments, R²¹ is selected from 5- to 6-membered heterocycle and C₃₋₆ carbocycle, any of which is substituted with C(O)H, wherein R²¹ is further optionally substituted with one or more substituents independently selected from: halogen, unsubstituted alkyl, unsubstituted haloalkyl, —OR¹⁵, —N(R¹⁵)C(O)R¹⁵, and —N(R¹⁵)C(O)N(R¹⁵)₂. In some embodiments, R²¹ is selected from 5- to 6-membered heterocycle and C₃₋₆ carbocycle, any of which is substituted with C(O)H, wherein R²¹ is further optionally substituted with one or more substituents independently selected from: halogen, unsubstituted alkyl, unsubstituted haloalkyl, —OR¹⁵, —N(R¹⁵)C(O)R¹⁵, and —N(R¹⁵)C(O)N(R¹⁵)₂; and R¹⁵ is independently selected at each occurrence from hydrogen, unsubstituted alkyl, unsubstituted carbocycle, unsubstituted heterocycle, and substituted heterocycle.

In some embodiments, for the compound or salt of Formula (I) or Formula (I-A), R²¹ is selected from heteroaryl and phenyl, any of which is substituted with C(O)H, wherein R²¹ is further optionally substituted with one or more substituents independently selected from: halogen, unsubstituted alkyl, unsubstituted haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN. In some embodiments, R²¹ is selected from heteroaryl and phenyl, any of which is substituted with C(O)H, wherein R²¹ is further optionally substituted with one or more substituents independently selected from: halogen, unsubstituted alkyl, unsubstituted haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN; and wherein R¹⁵ is independently selected at each occurrence from hydrogen, unsubstituted alkyl, unsubstituted carbocycle, unsubstituted heterocycle, and substituted heterocycle. In some embodiments, R²¹ is selected from heteroaryl and phenyl, any of which is substituted with C(O)H, wherein R²¹ is further optionally substituted with one or more substituents independently selected from: halogen, unsubstituted alkyl, unsubstituted haloalkyl, —OR¹⁵, —C(O)R¹⁵, —C(O)N(R¹⁵)₂, —C(O)OR¹⁵, —OC(O)R¹⁵, —N(R¹⁵)C(O)R¹⁵, —N(R¹⁵)C(O)N(R¹⁵)₂, ═O, and —CN. In some embodiments, R²¹ is selected from heteroaryl and phenyl, any of which is substituted with C(O)H, wherein R²¹ is further optionally substituted with one or more substituents independently selected from: halogen, unsubstituted alkyl, unsubstituted haloalkyl, —OR¹⁵, —N(R¹⁵)C(O)R¹⁵, and —N(R¹⁵)C(O)N(R¹⁵)₂. In some embodiments, R²¹ is selected from heteroaryl and phenyl, any of which is substituted with C(O)H, wherein R²¹ is further optionally substituted with one or more substituents independently selected from: halogen, unsubstituted alkyl, unsubstituted haloalkyl, —OR¹⁵, —N(R¹⁵)C(O)R¹⁵ and —N(R¹⁵)C(O)N(R¹⁵)₂; and R¹⁵ is independently selected at each occurrence from hydrogen, unsubstituted alkyl, unsubstituted carbocycle, unsubstituted heterocycle, and substituted heterocycle.

In some embodiments, for the compound or salt of Formula (I) or Formula (I-A), R²¹ is selected from pyridinyl and phenyl, any of which is substituted with C(O)H, wherein R²¹ is further optionally substituted with one or more substituents independently selected from: halogen, unsubstituted alkyl, unsubstituted
haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN. In some embodiments, R²¹ is selected from pyridinyl and phenyl, any of which is substituted with C(O)H, wherein R²¹ is further optionally substituted with one or more substituents independently selected from: halogen, unsubstituted alkyl, unsubstituted haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN; and wherein R¹⁵ is independently selected at each occurrence from hydrogen, unsubstituted alkyl, unsubstituted carbocycle, unsubstituted heterocycle, and substituted heterocycle. In some embodiments, R²¹ is selected from pyridinyl and phenyl, any of which is substituted with C(O)H, wherein R²¹ is further optionally substituted with one or more substituents independently selected from: halogen, unsubstituted alkyl, unsubstituted haloalkyl, —OR¹⁵, —C(O)R¹⁵, —C(O)N(R¹⁵)₂, —C(O)OR¹⁵, —OC(O)R¹⁵, —N(R¹⁵)C(O)R¹⁵, —N(R¹⁵)C(O)N(R¹⁵)₂, ═O, and —CN. In some embodiments, R²¹ is selected from pyridinyl and phenyl, any of which is substituted with C(O)H, wherein R²¹ is further optionally substituted with one or more substituents independently selected from: halogen, unsubstituted alkyl, unsubstituted haloalkyl, —OR¹⁵, —N(R¹⁵)C(O)R¹⁵, and —N(R¹⁵)C(O)N(R¹⁵)₂. In some embodiments, R²¹ is selected from pyridinyl and phenyl, any of which is substituted with C(O)H, wherein R²¹ is further optionally substituted with one or more substituents independently selected from: halogen, unsubstituted alkyl, unsubstituted haloalkyl, —OR¹⁵, —N(R¹⁵)C(O)R¹⁵ and —N(R¹⁵)C(O)N(R¹⁵)₂; and R¹⁵ is independently selected at each occurrence from hydrogen, unsubstituted alkyl, unsubstituted carbocycle, unsubstituted heterocycle, and substituted heterocycle.

In some embodiments, for the compound or salt of Formula (I) or Formula (I-A), R¹⁰, R¹¹, and R¹⁴ are each independently selected at each occurrence from hydrogen, alkyl, heteroalkyl, carbocycle, and heterocycle, any of which is independently unsubstituted or substituted. In some embodiments, R¹⁰, R¹¹, and R¹⁴ are each independently selected at each occurrence from hydrogen, unsubstituted alkyl, and substituted alkyl. In some embodiments, R¹⁰, R¹¹, and R¹⁴ are each hydrogen.

In some embodiments, for the compound or salt of Formula (I) or Formula (I-A), R¹⁰ is independently selected at each occurrence from hydrogen, alkyl, heteroalkyl, carbocycle, and heterocycle, any of which is independently unsubstituted or substituted. In some embodiments, R¹⁰ is independently selected at each occurrence from hydrogen, unsubstituted alkyl, and substituted alkyl. In some embodiments, R¹⁰ is hydrogen.

In some embodiments, for the compound or salt of Formula (I) or Formula (I-A), R¹¹ is independently selected at each occurrence from hydrogen, alkyl, heteroalkyl, carbocycle, and heterocycle, any of which is independently unsubstituted or substituted. In some embodiments, R¹¹ is independently selected at each occurrence from hydrogen, unsubstituted alkyl, and substituted alkyl. In some embodiments, R¹¹ is hydrogen.

In some embodiments, for the compound or salt of Formula (I) or Formula (I-A), R¹⁴, is independently selected at each occurrence from hydrogen, alkyl, heteroalkyl, carbocycle, and heterocycle, any of which is independently unsubstituted or substituted. In some embodiments, R¹⁴ is independently selected at each occurrence from hydrogen, unsubstituted alkyl, and substituted alkyl. In some embodiments, R¹⁴ is hydrogen.

In one aspect, the present disclosure provides a compound represented by the structure of Formula (II):

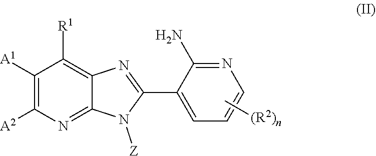

or a pharmaceutically acceptable salt thereof; wherein:
   R¹ and R² are each independently selected from hydrogen, halogen, C₁₋₄ alkyl, C₁₋₄ haloalkyl, —OR¹⁰, —SR¹⁰, —N(R¹⁰)₂, —NO₂, and —CN;
   n is selected from 0, 1, 2, and 3;
   A¹ and A² are each independently selected from:
      hydrogen,
         halogen, —OR¹¹, —SR¹¹, —N(R¹¹)₂, —C(O)R¹¹, —C(O)N(R¹¹)₂, —N(R¹¹)C(O)R¹¹, —NO₂, and —CN;
      C₁₋₆ alkyl, C₂₋₆ alkyenyl, and C₂₋₆ alkynyl, any one of which is optionally substituted with one or more substituents independently selected from halogen, —OR¹¹, —SR¹¹
         —N(R¹¹)₂, —C(O)N(R¹¹)₂, —N(R¹¹)C(O)R¹¹, ═O, ═S, ═N(R¹¹), and —CN; and
      3- to 10-membered heterocycle and C₃₋₁₀ carbocycle, any one of which is optionally substituted with one or more substituents independently selected from:
         halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
         C₁₋₆ alkyl, C₂₋₆ alkenyl, and C₂₋₆ alkynyl, any one of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹,
         —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), and —CN; and
         C₃₋₁₀ carbocycle and 3- to 10-membered heterocycle, any one of which is optionally substituted with one or more substituents independently selected from halogen, C₁₋₄ alkyl, C₁₋₄ haloalkyl, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O;
   Z is represented by

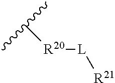

wherein,
R²⁰ is selected from 5- to 6-membered heterocyclene and phenylene, any one of which is optionally substituted with one or more substituents independently selected from halogen, C₁₋₄ alkyl, C₁₋₄ haloalkyl —OR¹², —SR¹², —N(R¹²)₂, —NO₂, ═O, ═S, ═N(R¹²), and —CN;
L is represented by -L¹-L²-L³-L⁴-, wherein each L¹, L², L³, and L⁴ is independently selected from (a) and (b):
   (a) —O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, (R¹⁴)NC(O)N(R¹⁴), and (R¹⁴)NC(O)N(R¹⁴)N(R¹⁴); and
   (b) C₁₋₆ alkylene, C₂₋₆ alkenylene, C₂₋₆ alkynylene, C₃₋₈ carbocyclene, and 3to 8-membered heterocyclene, any one of which is optionally substituted with one or more substituents independently selected from halogen, —OR¹³, —SR¹³, —N(R¹³)₂, ═O, ═S, and —CN;
   wherein L¹, L², or L³ are each optionally absent;
   wherein no more than two of L¹, L², L³, and L⁴ are selected from (a) and the two selected are not adjacent to each other;
R²¹ is selected from 5- to 6-membered heterocycle and C₃₋₆ carbocycle, any one of which is substituted with —C(O)H, wherein R²¹ is further optionally substituted with one or more substituents independently selected from:
   halogen, C₁₋₄ alkyl, C₁₋₄
   haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN; and
R¹⁰, R¹¹, R¹², R¹³, R¹⁴, and R¹⁵ are each independently selected at each occurrence from hydrogen, C₁₋₄ alkyl, C₁₋₄ haloalkyl, C₃-s carbocycle, and 3- to 8-membered heterocycle, wherein the C₃-s carbocycle and 3- to 8-membered heterocycle are each optionally substituted with one or more substituents independently selected from halogen, C₁₋₄ alkyl, C₁₋₄ haloalkyl, and ═O.

In some embodiments, for the compound or salt of Formula (II), R¹ is selected from hydrogen, halogen, C₁₋₄ alkyl, C₁₋₄ haloalkyl, —OR¹⁰, —SR¹⁰, —N(R¹⁰)₂, —NO₂, and —CN. In some embodiments, R¹ is selected from hydrogen, halogen, C₁₋₄ alkyl, and C₁₋₄ haloalkyl. In some embodiments, R¹ is hydrogen.

In some embodiments, for the compound or salt of Formula (II), R² is selected from halogen, C₁₋₄ alkyl, C₁₋₄ haloalkyl, —OR¹⁰, —SR¹⁰, —N(R¹⁰)₂, —NO₂, and —CN. In some embodiments, R² is selected from halogen, C₁₋₄ alkyl, C₁₋₄ haloalkyl, —OR¹⁰, —SR¹⁰, —N(R¹⁰)₂, —NO₂, and —CN; and R¹⁰ is independently selected at each occurrence from hydrogen and C₁₋₄ alkyl. In some embodiments, R² is selected from halogen, C₁₋₄ alkyl, C₁₋₄ haloalkyl, —OR¹⁰, —SR¹⁰, —N(R¹⁰)₂, —NO₂, and —CN; and R¹⁰ is hydrogen. In some embodiments, R² is selected from halogen, C₁₋₄ alkyl, and C₁₋₄ haloalkyl.

In some embodiments, for the compound or salt of Formula (II), n is selected from 0, 1, 2, and 3. In some embodiments, n is selected from 0, 1, and 2. In some embodiments, n is selected from 0 and 1. In some embodiments, n is 0.

In some embodiments, the compound or salt of Formula (II) is a compound represented by the structure of Formula (II-A):

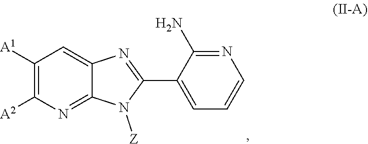

or a pharmaceutically acceptable salt thereof, wherein A¹, A², and Z are each defined as in Formula (II).

In some embodiments, for the compound or salt of Formula (II) or (II-A), A¹ and A² are each independently selected from:
hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, and —CN;
C₁₋₆ alkyl, C₂₋₆ alkyenyl, and C₂₋₆ alkynyl, any one of which is optionally substituted with one or more substituents independently selected from halogen, —OR¹¹, —SR¹¹, —N(R¹¹)₂, —C(O)N(R¹¹)₂, —N(R¹¹)C(O)R¹¹, ═O, ═S, ═N(R¹¹), and —CN; and
   3- to 10-membered heterocycle and C₃₋₁₀ carbocycle, any one of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₂₋₆ alkynyl, any one of which is optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), and —CN; and
   C₃₋₁₀ carbocycle and 3- to 10-membered heterocycle, any one of which is optionally substituted with one or more substituents independently selected from halogen, C₁₋₄ alkyl, C₁₋₄ haloalkyl, —OR¹¹, —N(R¹¹)₂,
      —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O.

In some embodiments, for the compound or salt of Formula (II) or (II-A), A¹ and A² are each independently selected from:
hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, and —CN;
C₁₋₆ alkyl, C₂₋₆ alkyenyl, and C₂₋₆ alkynyl, any one of which is optionally substituted with one or more substituents independently selected from halogen, —OR¹¹, —SR¹¹, —N(R¹¹)₂, —C(O)N(R¹¹)₂, —N(R¹¹)C(O)R¹¹, ═O, ═S, ═N(R¹¹), and —CN; and
3- to 10-membered heterocycle and C₃₋₁₀ carbocycle, any one of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₂₋₆ alkynyl, any one of which is optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), and —CN; and
   C₃₋₁₀ carbocycle and 3- to 10-membered heterocycle, any one of which is optionally substituted with one or more substituents independently selected from halogen, C₁₋₄ alkyl, C₁₋₄ haloalkyl, —OR¹¹, —N(R¹¹)₂,
      —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O; and
   R¹¹ is independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O.

In some embodiments, for the compound or salt of Formula (II) or (II-A), A¹ and A² are each independently selected from:
hydrogen, halogen, —OR¹¹, —N(R¹¹)₂, —C(O)N(R¹¹)₂, and —N(R¹¹)C(O)R¹¹; and
C₁₋₆ alkyl, C₂₋₆ alkyenyl, and C₂₋₆ alkynyl, any one of which is optionally substituted with one or more substituents independently selected from halogen, —OR¹¹, —N(R¹¹)₂, —C(O)N(R¹¹)₂, —N(R¹¹)C(O)R¹¹, and ═O; and 3- to 10-membered heterocycle and C₃₋₁₀ carbocycle, any one of which is optionally substituted with one or more substituents independently selected from:
   halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, C₁₋₆ alkyl, and C₁₋₆ haloalkyl; and
   C₃₋₁₀ carbocycle and 3- to 10-membered heterocycle, any one of which is optionally substituted with one or more substituents independently selected from halogen, C₁₋₄ alkyl, C₁₋₄ haloalkyl, —OR¹¹, —N(R¹¹)₂, —N(R¹¹)C(O)R¹¹, ═O, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O.

In some embodiments, for the compound or salt of Formula (II) or (II-A), A¹ and A² are each independently selected from:
hydrogen, halogen, —OR¹¹, and —N(R¹¹)₂;
C₁₋₆ alkyl and C₂₋₆ alkynyl, any one of which is optionally substituted with one or more substituents independently selected from halogen and —OR¹¹; and 3- to 10-membered heterocycle and C₃₋₁₀ carbocycle, any one of which is optionally substituted with one or more substituents independently selected from:
   halogen, —C(O)R¹¹, —N(R¹¹)C(O)R¹¹, —N(R¹¹)S(O)₂R¹¹, C₁₋₆ alkyl, and 3- to 10-membered heterocycle, wherein the 3- to 10-membered heterocycle is optionally substituted with one or more substituents independently selected from —N(R¹¹)C(O)R¹¹ and 3- to 6-membered heterocycle optionally substituted with one or more ═O.

In some embodiments, for the compound or salt of Formula (II) or (II-A), A¹ and A² are each independently selected from:
hydrogen, halogen, —OR¹¹, and —N(R¹¹)₂;
C₁₋₆ alkyl and C₂₋₆ alkynyl, any one of which is optionally substituted with one or more substituents independently selected from halogen and —OR¹¹; and
3- to 10-membered heterocycle and C₃₋₁₀ carbocycle, any one of which is optionally substituted with one or more substituents independently selected from:
   halogen, —C(O)R¹¹, —N(R¹¹)C(O)R¹¹, —N(R¹¹)S(O)₂R¹¹, C₁₋₆ alkyl, and 3- to 10-membered heterocycle, wherein the 3- to 10-membered heterocycle is optionally substituted with one or more substituents independently selected
   from —N(R¹¹)C(O)R¹¹ and 3- to 6-membered heterocycle optionally substituted with one or more ═O; and
R¹¹ is independently selected at each occurrence from hydrogen, C₁₋₄ alkyl, C₃₋₈ carbocycle, and 3- to 8-membered heterocycle, wherein the C₃₋₈ carbocycle and 3- to 8-membered heterocycle are each optionally substituted with one or more C₁₋₄ alkyl.

In some embodiments, for the compound or salt Formula (II) or (II-A), A¹ and A² are each independently selected from:
hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, and —CN; and
C₁₋₆ alkyl, C₂₋₆ alkyenyl, and C₂₋₆ alkynyl, any one of which is optionally substituted with one or more substituents independently selected from halogen, —OR¹¹, —SR¹¹, —N(R¹¹)₂, —C(O)N(R¹¹)₂, —N(R¹¹)C(O)R¹¹, ═O, ═S, ═N(R¹¹), and —CN.

In some embodiments, for the compound or salt Formula (II) or (II-A), A¹ and A² are each independently selected from:
hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, and —CN; and
C₁₋₆ alkyl, C₂₋₆ alkyenyl, and C₂₋₆ alkynyl, any one of which is optionally substituted with one or more substituents independently selected from halogen, —OR¹¹, —SR¹¹, —N(R¹¹)₂, —C(O)N(R¹¹)₂, —N(R¹¹)C(O)R¹¹, ═O, ═S, ═N(R¹¹), and —CN.

In some embodiments, for the compound or salt of Formula (II) or (II-A), A¹ and A² are each independently selected from:
hydrogen, halogen, —OR¹¹, —N(R¹¹)₂, —C(O)R¹¹, —C(O)N(R¹¹)₂, and —N(R¹¹)C(O)R¹¹; and
C₁₋₆ alkyl, C₂₋₆ alkyenyl, and C₂₋₆ alkynyl, any one of which is optionally substituted with one or more substituents independently selected from halogen, —OR¹¹, —N(R¹¹)₂, —C(O)N(R¹¹)₂, —N(R¹¹)C(O)R¹¹, and ═O.

In some embodiments, for the compound or salt of Formula (II) or (II-A), A¹ and A² are each independently selected from:
hydrogen, halogen, —OR¹¹, —N(R¹¹)₂, —C(O)R¹¹, —C(O)N(R¹¹)₂, and —N(R¹¹)C(O)R¹¹; and
C₁₋₆ alkyl, C₂₋₆ alkyenyl, and C₂₋₆ alkynyl, any one of which is optionally substituted with one or more substituents independently selected from halogen, —OR¹¹, —N(R¹¹)₂, —C(O)N(R¹¹)₂, —N(R¹¹)C(O)R¹¹, and ═O; and
R¹¹ is independently selected from: hydrogen, C₁₋₃ alkyl, C₁₋₃ haloalkyl, and C₃₋₄ carbocycle.

In some embodiments, for the compound or salt of Formula (II) or (II-A), A¹ and A² are each independently selected from hydrogen, halogen, —OR¹¹, —N(R¹¹)₂, C₁₋₆ alkyl, and C₂₋₆ alkynyl, wherein the C₁₋₆ alkyl, and C₂₋₆ alkynyl are each optionally substituted with one or more substituents independently selected from halogen and —OR¹¹. In some embodiments, A¹ and A² are hydrogen.

In some embodiments, for the compound or salt of Formula (II) or (II-A), A¹ is independently selected from:
hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, and —CN;
C₁₋₆ alkyl, C₂₋₆ alkyenyl, and C₂₋₆ alkynyl, any one of which is optionally substituted with one or more substituents independently selected from halogen, —OR¹¹, —SR¹¹, —N(R¹¹)₂, —C(O)N(R¹¹)₂, —N(R¹¹)C(O)R¹¹, ═O, ═S, ═N(R¹¹), and —CN; and
3- to 10-membered heterocycle and C₃₋₁₀ carbocycle, any one of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₂₋₆ alkynyl, any one of which is optionally substituted with one or more substituents independently selected from: halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), and —CN; and
   C₃₋₁₀ carbocycle and 3- to 10-membered heterocycle, any one of which is optionally substituted with one or more substituents independently selected from halogen, C₁₋₄ alkyl, C₁₋₄ haloalkyl, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O.

In some embodiments, for the compound or salt of Formula (II) or (II-A), A¹ is independently selected from:
hydrogen, halogen, —OR¹¹, and —CN;
C₁₋₆ alkyl optionally substituted with one or more substituents independently selected from halogen, —OR¹¹, ═O, and —CN; and
3- to 10-membered heterocycle and C₃₋₁₀ carbocycle, any one of which is optionally substituted with one or more substituents independently selected from halogen, —OR¹¹, ═O, —CN, and C₁₋₆ alkyl.

In some embodiments, for the compound or salt of Formula (II) or (II-A), A¹ is selected from hydrogen, halogen, 3- to 10-membered heterocycle, and C₃₋₁₀ carbocycle. In some embodiments, A¹ is selected from hydrogen and C₃₋₁₀ carbocycle. In some embodiments, A¹ is selected from hydrogen and C₃₋₆ carbocycle. In some embodiments, A¹ is selected from hydrogen and phenyl. In some embodiments, A¹ is hydrogen.

In some embodiments, the compound or salt of Formula (II) is a compound represented by the structure of Formula (II-B):

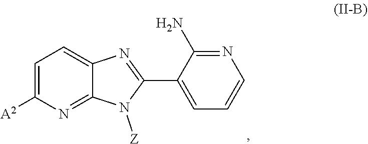

or a pharmaceutically acceptable salt thereof, wherein A² and Z are each defined as in Formula (II).

In some embodiments, for the compound or salt of Formula (II), (II-A), or (II-B), A² is selected from:
hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, and —CN;
C₁₋₆ alkyl, C₂₋₆ alkyenyl, and C₂₋₆ alkynyl, any one of which is optionally substituted with one or more substituents independently selected from halogen, —OR¹¹, —SR¹¹, —N(R¹¹)₂, —C(O)N(R¹¹)₂, —N(R¹¹)C(O)R¹¹, ═O, ═S, ═N(R¹¹), and —CN; and
3- to 10-membered heterocycle and C₃₋₁₀ carbocycle, any one of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₂₋₆ alkynyl, any one of which is optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), and —CN; and
   C₃₋₁₀ carbocycle and 3- to 10-membered heterocycle, any one of which is optionally substituted with one or more substituents independently selected from halogen, C₁₋₄ alkyl, C₁₋₄ haloalkyl, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O.

In some embodiments, for the compound or salt of Formula (II), (II-A), or (II-B), A² is selected from:
hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, and —CN;
C₁₋₆ alkyl, C₂₋₆ alkyenyl, and C₂₋₆ alkynyl, any one of which is optionally substituted with one or more substituents independently selected from halogen, —OR¹¹, —SR¹¹, —N(R¹¹)₂, —C(O)N(R¹¹)₂, —N(R¹¹)C(O)R¹¹, ═O, ═S, ═N(R¹¹), and —CN; and
3- to 10-membered heterocycle and C₃₋₁₀ carbocycle, any one of which is optionally substituted with one or more substituents independently selected from:
   halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
   C₁₋₆ alkyl, C₂₋₆ alkenyl, and C₂₋₆ alkynyl, any one of which is optionally substituted with one or more substituents independently selected from:
      halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), and —CN; and
   C₃₋₁₀ carbocycle and 3- to 10-membered heterocycle, any one of which is optionally substituted with one or more substituents independently selected from halogen, C₁₋₄ alkyl, C₁₋₄ haloalkyl, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O; and
   R¹¹ is independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O.

In some embodiments, for the compound or salt of Formula (II), (II-A), or (II-B), A² is selected from:
hydrogen, halogen, —OR¹¹, —N(R¹¹)₂, —C(O)R¹¹, —C(O)N(R¹¹)₂, and —N(R¹¹)C(O)R¹¹;
C₁₋₆ alkyl, C₂₋₆ alkyenyl, and C₂₋₆ alkynyl, any one of which is optionally substituted with one or more substituents independently selected from halogen, —OR¹¹, —N(R¹¹)₂, —C(O)N(R¹¹)₂, —N(R¹¹)C(O)R¹¹, and ═O; and
3- to 10-membered heterocycle and C₃₋₁₀ carbocycle, any one of which is optionally substituted with one or more substituents independently selected from:
   halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, C₁₋₆ alkyl, and C₁₋₆ haloalkyl; and
   C₃₋₁₀ carbocycle and 3- to 10-membered heterocycle, any one of which is optionally substituted with one or more substituents independently selected from halogen, C₁₋₄ alkyl, C₁₋₄ haloalkyl, —OR¹¹, —N(R¹¹)₂, —N(R¹¹)C(O)R¹¹, ═O, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O.

In some embodiments, for the compound or salt of Formula (II), (II-A), or (II-B), A² is selected from:
hydrogen, halogen, —OR¹¹, and —N(R¹¹)₂;
C₁₋₆ alkyl and C₂₋₆ alkynyl, any one of which is optionally substituted with one or more substituents independently selected from halogen and —OR¹¹; and
3- to 10-membered heterocycle and C₃₋₁₀ carbocycle, any one of which is optionally substituted with one or more substituents independently selected from:
   halogen, —C(O)R¹¹, —N(R¹¹)C(O)R¹¹, —N(R¹¹)S(O)₂R¹¹, and C₁₋₆ alkyl; and
   3- to 10-membered heterocycle optionally substituted with one or more substituents independently selected from —N(R¹¹)C(O)R¹¹ and 3- to 6-membered heterocycle, wherein the 3- to 6-membered heterocycle is optionally substituted with ═O.

In some embodiments, for the compound or salt of Formula (II), (II-A), or (II-B), A² is selected from:
hydrogen, halogen, —OR¹¹, and —N(R¹¹)₂;
C₁₋₆ alkyl and C₂₋₆ alkynyl, any one of which is optionally substituted with one or more substituents independently selected from halogen and —OR¹¹; and
3- to 10-membered heterocycle and C₃₋₁₀ carbocycle, any one of which is optionally substituted with one or more substituents independently selected from:
   halogen, —C(O)R¹¹, —N(R¹¹)C(O)R¹¹, —N(R¹¹)S(O)₂R¹¹, and C₁₋₆ alkyl; and
   3- to 10-membered heterocycle optionally substituted with one or more substituents independently selected from —N(R¹¹)C(O)R¹¹ and 3- to 6-membered heterocycle, wherein the 3- to 6-membered heterocycle is optionally substituted with ═O; and
   R¹¹ is independently selected at each occurrence from hydrogen, C₁₋₄ alkyl, C₃₋₈ carbocycle, and 3- to 8-membered heterocycle, wherein the C₃₋₈ carbocycle and 3- to 8-membered heterocycle are each optionally substituted with one or more C₁₋₄ alkyl.

In some embodiments, for the compound or salt Formula (II), (II-A), or (II-B), A² is selected from:
hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, and —CN; and
C₁₋₆ alkyl, C₂₋₆ alkyenyl, and C₂₋₆ alkynyl, any one of which is optionally substituted with one or more substituents independently selected from halogen, —OR¹¹, —SR¹¹, —N(R¹¹)₂, —C(O)N(R¹¹)₂, —N(R¹¹)C(O)R¹¹, ═O, ═S, ═N(R¹¹), and —CN.

In some embodiments, for the compound or salt of Formula (II), (II-A), or (II-B), A² is selected from:
hydrogen, halogen, —OR¹¹, —N(R¹¹)₂, —C(O)R¹¹, —C(O)N(R¹¹)₂, and —N(R¹¹)C(O)R¹¹; and
C₁₋₆ alkyl, C₂₋₆ alkyenyl, and C₂₋₆ alkynyl, any one of which is optionally substituted with one or more substituents independently selected from halogen, —OR¹¹, —N(R¹¹)₂, —C(O)N(R¹¹)₂, —N(R¹¹)C(O)R¹¹, and ═O.

In some embodiments, for the compound or salt of Formula (II), (II-A), or (II-B), A² is selected from:
hydrogen, fluoro, chloro, bromo, —OR¹¹, —N(R¹¹)₂; and C₁₋₄ alkyl and C₂₋₄ alkynyl, any of which is optionally substituted with one or more substituents independently selected from halogen and —OR¹¹; and
R¹¹ is independently selected from: hydrogen, C₁₋₃ alkyl, C₁₋₃ haloalkyl, and C₃₋₄ carbocycle.

In some embodiments, for the compound or salt of Formula (II), (II-A), or (II-B), A² is selected from hydrogen, halogen, —OR¹¹, —N(R¹¹)₂, C₁₋₆ alkyl, and C₂₋₆ alkynyl, wherein the C₁₋₆ alkyl, and C₂₋₆ alkynyl are each optionally substituted with one or more substituents independently selected from halogen and —OR¹¹. In some embodiments, A² is selected from hydrogen, halogen, —OR¹¹, —N(R¹¹)₂, C₁₋₆ alkyl, and C₂₋₆ alkynyl, wherein the C₁₋₆ alkyl, and C₂₋₆ alkynyl are each optionally substituted with one or more substituents independently selected from halogen and —OR¹¹; and R¹¹ is independently selected at each occurrence from hydrogen, C₁₋₄ alkyl, C₃-s carbocycle, and 3- to 8-membered heterocycle, wherein the C₃₋₈ carbocycle and 3- to 8-membered heterocycle are each optionally substituted with one or more C₁₋₄ alkyl. In some embodiments, In some embodiments, A² is hydrogen.

In some embodiments, for the compound or salt of Formula (II), (II-A), or (II-B), A² is selected from hydrogen, fluoro, chloro,

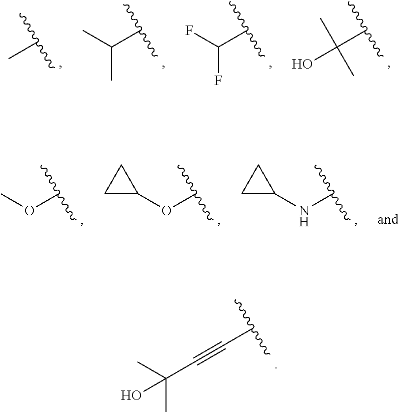

In some embodiments, for the compound or salt of Formula (II), (II-A), or (II-B), A² is selected from 3- to 10-membered heterocycle and C₃₋₁₀ carbocycle, any one of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
C₁₋₆ alkyl, C₂₋₆ alkenyl, and C₂₋₆ alkynyl, any one of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), and —CN; and
C₃₋₁₀ carbocycle and 3- to 10-membered heterocycle; any one of which is optionally substituted with one or more substituents independently selected from halogen, C₁₋₄ alkyl, C₁₋₄ haloalkyl, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O.

In some embodiments, for the compound or salt of Formula (II), (II-A), or (II-B), A² is selected from 3- to 10-membered heterocycle and C₃₋₁₀ carbocycle, any one of which is optionally substituted with one or more substituents independently selected from:
halogen, —OR¹¹, —N(R¹¹)₂, —C(O)R¹¹, —C(O)N(R¹¹)₂, —N(R¹¹)C(O)R¹¹, —N(R¹¹)S(O)₂R¹¹, C₁₋₆ alkyl, and C₁₋₆ haloalkyl; and
C₃₋₁₀ carbocycle and 3- to 10-membered heterocycle; any one of which is optionally substituted with one or more substituents independently selected from halogen, C₁₋₄ alkyl, C₁₋₄ haloalkyl, —OR¹¹, —N(R¹¹)₂, —N(R¹¹)C(O)R¹¹, ═O, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O.

In some embodiments, for the compound or salt of Formula (II), (II-A), or (II-B), A² is selected from 3- to 10-membered heterocycle and C₃₋₁₀ carbocycle, any one of which is optionally substituted with one or more substituents independently selected from:
halogen, —C(O)R¹¹, —N(R¹¹)C(O)R¹¹, —N(R¹¹)S(O)₂R¹¹, and C₁₋₆ alkyl; and
3- to 10-membered heterocycle optionally substituted with one or more substituents independently selected from —N(R¹¹)C(O)R¹¹ and 3- to 6-membered heterocycle, wherein the 3- to 6-membered heterocycle is optionally substituted with one or more ═O.

In some embodiments, for the compound or salt of Formula (II), (II-A), or (II-B), A² is selected from 3- to 10-membered heterocycle and C₃₋₁₀ carbocycle, any one of which is optionally substituted with one or more substituents independently selected from:
halogen, —C(O)R¹¹, —N(R¹¹)C(O)R¹¹, —N(R¹¹)S(O)₂R¹¹, and C₁₋₆ alkyl; and
3- to 10-membered heterocycle optionally substituted with one or more substituents independently selected from —N(R¹¹)C(O)R¹¹ and 3- to 6-membered heterocycle, wherein the 3- to 6-membered heterocycle is optionally substituted with one or more ═O; and
R¹¹ is independently selected at each occurrence from hydrogen, C₁₋₄ alkyl, C₃₋₈ carbocycle, and 3- to 8-membered heterocycle, wherein the C₃₋₈ carbocycle and 3- to 8-membered heterocycle are each optionally substituted with one or more C₁₋₄ alkyl.

In some embodiments, for the compound or salt of Formula (II), (II-A), or (II-B), A² is selected from 3- to 10-membered heterocycle and C₃₋₁₀ carbocycle, one of which is optionally substituted with one or more substituents independently selected from methyl, fluoro,

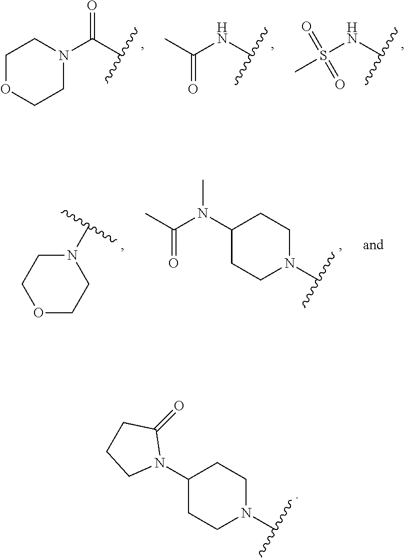

In some embodiments, for the compound or salt of Formula (II), (II-A), or (II-B), A² is selected from cyclopropyl, phenyl, pyrazolyl, morpholine, pyridinyl, 8-Oxa-3-azabicyclo[3.2.1]octanyl, 3-Oxa-8-azabicyclo[3.2.1]octanyl any one of which is optionally substituted with one or more substituents independently selected from halogen, C₁₋₃ alkyl, —C(O)N(R¹¹)₂, —N(R¹¹)C(O)R¹¹, —N(R¹¹)S(O)₂R¹¹, and 3- to 10-membered heterocycle, wherein the 3- to 10-membered heterocycle is optionally substituted with one or more substituents independently selected from —N(R¹¹)C(O)R¹¹ and 5-membered heterocycle, wherein the 5-membered heterocycle is optionally substituted with ═O.

In some embodiments, for the compound or salt of Formula (II), (II-A), or (II-B), A² is selected from cyclopropyl, phenyl, pyrazolyl, morpholine, pyridinyl, 8-Oxa-3-azabicyclo[3.2.1]octanyl, 3-Oxa-8-azabicyclo[3.2.1]octanyl any one of which is optionally substituted with one or more substituents independently selected from halogen, C₁₋₃ alkyl, —N(R¹¹)S(O)₂R¹¹, and 3- to 10-membered heterocycle, wherein the 3- to 10-membered heterocycle is optionally substituted with one or more substituents independently selected from —N(R¹¹)C(O)R¹¹ and 5-membered heterocycle, wherein the 5-membered heterocycle is optionally substituted with ═O.

In some embodiments, for the compound or salt of Formula (II), (II-A), or (II-B), A² is selected from cyclopropyl, phenyl, pyrazolyl, morpholine, pyridinyl, 8-Oxa-3-azabicyclo[3.2.1]octanyl, 3-Oxa-8-azabicyclo[3.2.1]octanyl any one of which is optionally substituted with one or more substituents independently selected from halogen, C₁₋₃ alkyl, —C(O)N(R¹¹)₂, —N(R¹¹)C(O)R¹¹, —N(R¹¹)S(O)₂R¹¹, morpholine, and piperidinyl, wherein the piperidinyl is optionally substituted with one or more substituents independently selected from —N(R¹¹)C(O)R¹¹ and 5-membered heterocycle, wherein the 5-membered heterocycle is optionally substituted with ═O.

In some embodiments, for the compound or salt of Formula (II), (II-A), or (II-B), A² is selected from cyclopropyl, phenyl, pyrazolyl, morpholine, pyridinyl, 8-Oxa-3-azabicyclo[3.2.1]octanyl, 3-Oxa-8-azabicyclo[3.2.1]octanyl any one of which is optionally substituted with one or more substituents independently selected from halogen, C₁₋₃ alkyl, —N(R¹¹)S(O)₂R¹¹, morpholine, and piperidinyl, wherein the piperidinyl is optionally substituted with one or more substituents independently selected from —N(R¹¹)C(O)R¹¹ and 5-membered heterocycle, wherein the 5-membered heterocycle is optionally substituted with ═O.

In some embodiments, for the compound or salt of Formula (II), (II-A), or (II-B), A² is selected from cyclopropyl, phenyl, pyrazolyl, morpholine, pyridinyl, 8-Oxa-3-azabicyclo[3.2.1]octanyl, 3-Oxa-8-azabicyclo[3.2.1]octanyl any one of which is optionally substituted with one or more substituents independently selected from: halogen, C₁₋₃ alkyl, —C(O)N(R¹¹)₂, —N(R¹¹)C(O)R¹¹, —N(R¹¹)S(O)₂R¹¹, morpholine, and piperidinyl, wherein the piperidinyl is optionally substituted with one or more substituents independently selected from —N(R¹¹)C(O)R¹¹ and pyrrolidinone.

In some embodiments, for the compound or salt of Formula (II), (II-A), or (II-B), A² is selected from cyclopropyl, phenyl, pyrazolyl, morpholine, pyridinyl, 8-Oxa-3-azabicyclo[3.2.1]octanyl, 3-Oxa-8-azabicyclo[3.2.1]octanyl any one of which is optionally substituted with one or more substituents independently selected from: halogen, C₁₋₃ alkyl, —N(R¹¹)S(O)₂R¹¹, morpholine, and piperidinyl, wherein the piperidinyl is optionally substituted with one or more substituents independently selected from —N(R¹¹)C(O)R¹¹ and pyrrolidinone.

In some embodiments, for the compound or salt of Formula (II), (II-A), or (II-B), A² is selected from cyclopropyl, phenyl, pyrazolyl, morpholine, pyridinyl, 8-Oxa-3-azabicyclo[3.2.1]octanyl, 3-Oxa-8-azabicyclo[3.2.1]octanyl any one of which is optionally substituted with one or more substituents independently selected from: halogen, C₁₋₃ alkyl, —N(R¹¹)S(O)₂R¹¹, morpholine, and piperidinyl, wherein the piperidinyl is optionally substituted with one or more substituents independently selected from —N(R¹¹)C(O)R¹¹ and pyrrolidinone; and R¹¹ is independently selected at each occurrence from hydrogen and C₁₋₄ alkyl.

In some embodiments, for the compound or salt of Formula (II), (II-A), or (II-B), A² is selected from cyclopropyl, phenyl, pyrazolyl, morpholine, pyridinyl, 8-Oxa-3-azabicyclo[3.2.1]octanyl, 3-Oxa-8-azabicyclo[3.2.1]octanyl any one of which is optionally substituted with one or more substituents independently selected from methyl, fluoro,

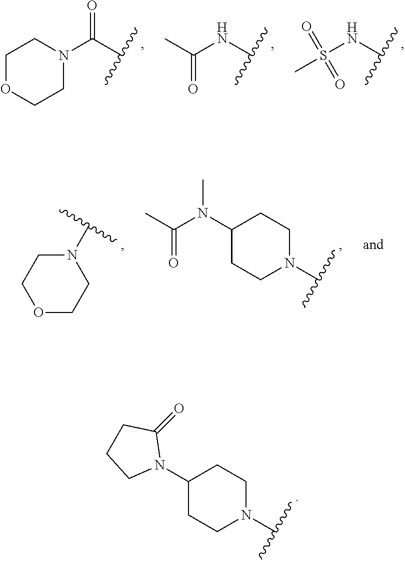

In some embodiments, for the compound or salt of Formula (II), (II-A), or (II-B), A² is selected from:

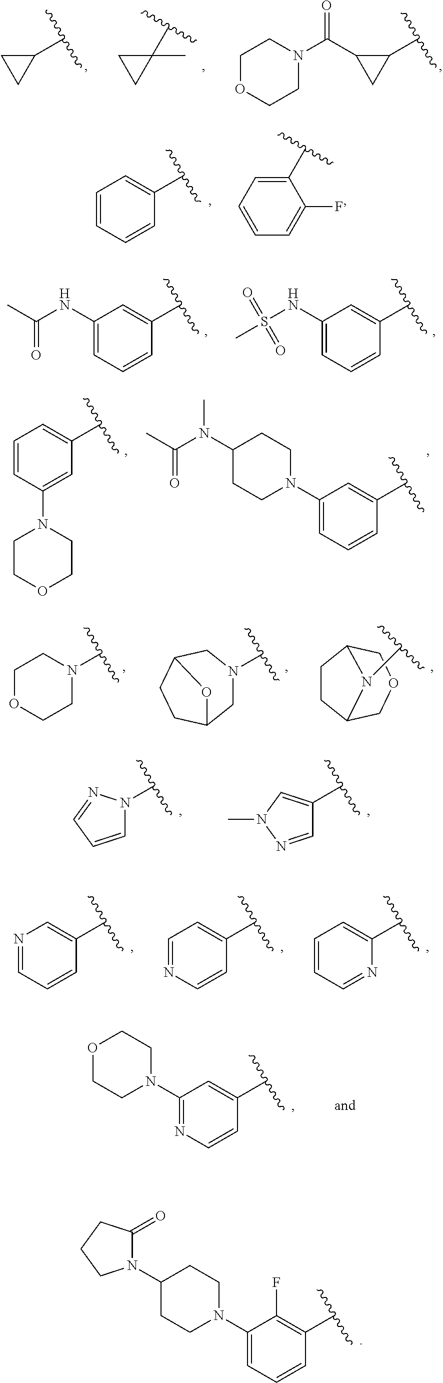

In some embodiments, for the compound or salt of Formula (II), (II-A) or (II-B) A² is selected from:

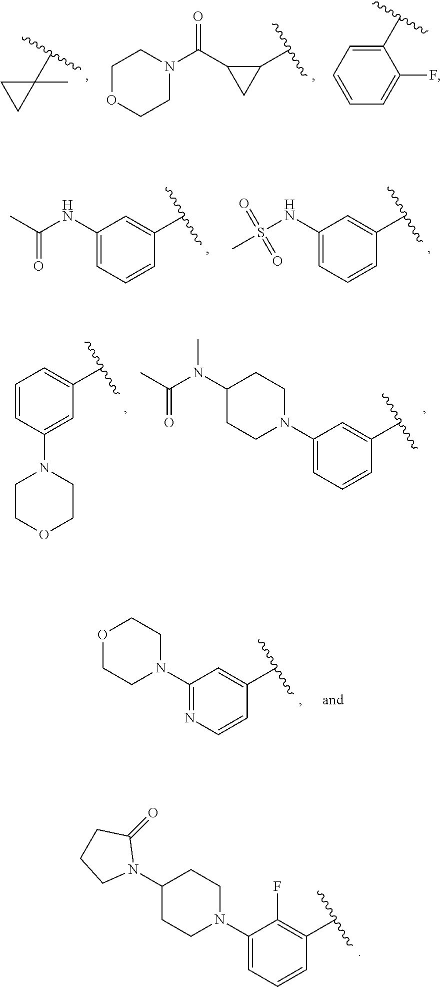

In some embodiments, for the compound or salt of Formula (II), (II-A), or (II-B), A² is selected from:

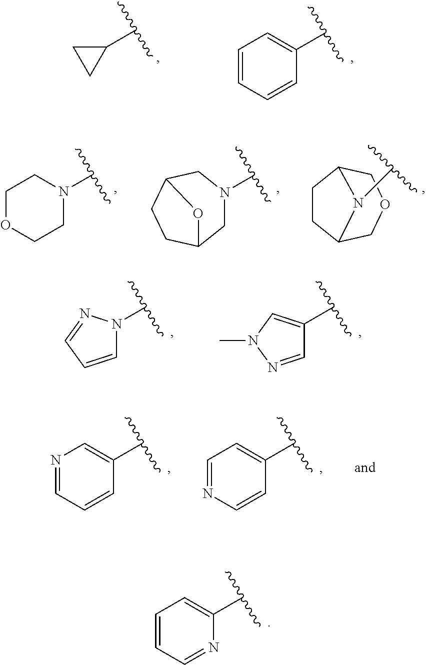

In some embodiments, for the compound or salt of Formula (II), (II-A), or (II-B), R²⁰ is selected from 5- to 6-membered heterocyclene and phenylene, any one of which is optionally substituted with one or more substituents independently selected from halogen, C₁₋₄ alkyl, C₁₋₄ haloalkyl, —OR¹², —SR¹², —N(R¹²)₂, —NO₂, ═O, ═S, ═N(R¹²), and —CN. In some embodiments, R²⁰ is selected from 5- to 6-membered heterocyclene and phenylene, any one of which is optionally substituted with one or more substituents independently selected from halogen, C₁₋₄ alkyl, C₁₋₄ haloalkyl, —OR¹², —SR¹²,
—N(R¹²)₂, —NO₂, ═O, ═S, ═N(R¹²), and —CN; and R¹² is independently selected at each occurrence from hydrogen, C₁₋₄ alkyl, C₁₋₄ haloalkyl, C₃₋₈ carbocycle, and 3- to 8-membered heterocycle, wherein the C₃-s carbocycle and 3- to 8-membered heterocycle are each optionally substituted with one or more substituents independently selected from halogen, C₁₋₄ alkyl, C₁₋₄ haloalkyl, and ═O.

In some embodiments, for the compound or salt of Formula (II), (II-A), or (II-B), R²⁰ is selected from 5- to 6-membered heterocyclene and phenylene, any one of which is optionally substituted with one or more substituents independently selected from halogen, C₁₋₄ alkyl, C₁₋₄ haloalkyl, and —CN. In some embodiments, R²⁰ is selected from 5- to 6-membered heterocyclene and phenylene.

In some embodiments, for the compound or salt of Formula (II), (II-A), or (II-B), R²⁰ is selected from 6-membered heteroarylene and phenylene, any one of which is optionally substituted with one or more substituents independently selected from halogen, C₁₋₄ alkyl, C₁₋₄ haloalkyl, —OR¹², —SR¹², —N(R¹²)₂, —NO₂, and —CN. In some embodiments, R²⁰ is selected from 6-membered heteroarylene and phenylene, any one of which is optionally substituted with one or more substituents independently selected from halogen, C₁₋₄ alkyl, C₁₋₄ haloalkyl, —OR¹², —SR¹², —N(R¹²)₂, —NO₂, and —CN; and R¹² is independently selected at each occurrence from hydrogen and C₁₋₄ alkyl.

In some embodiments, for the compound or salt of Formula (II), (II-A), or (II-B), R²⁰ is selected from 6-membered heteroarylene and phenylene, any one of which is optionally substituted with one or more substituents independently selected from halogen, C₁₋₄ alkyl, C₁₋₄ haloalkyl, and —CN. In some embodiments, R²⁰ is selected from 6-membered heteroarylene and phenylene.

In some embodiments, for the compound or salt of Formula (II), (II-A), or (II-B), R²⁰ is selected from pyridinylene and phenylene, any one of which is optionally substituted with one or more substituents independently selected from halogen, C₁₋₄ alkyl, C₁₋₄ haloalkyl, —OR¹², —SR¹², —N(R¹²)₂, —NO₂, and —CN. In some embodiments, R²⁰ is selected from pyridinylene and phenylene, any one of which is optionally substituted with one or more substituents independently selected from halogen, C₁₋₄ alkyl, C₁₋₄ haloalkyl, —OR¹², —SR¹², —N(R¹²)₂, —NO₂, and —CN; and R¹² is independently selected at each occurrence from hydrogen and C₁₋₄ alkyl.

In some embodiments, for the compound or salt of Formula (II), (II-A), or (II-B), R²⁰ is selected from pyridinylene and phenylene, any one of which is optionally substituted with one or more substituents independently selected from halogen, C₁₋₄ alkyl, C₁₋₄ haloalkyl, and —CN. In some embodiments, R²⁰ is selected from pyridinylene and phenylene. In some embodiments, R²⁰ is phenylene.

In some embodiments, for the compound or salt of Formula (II), (II-A), or (II-B), R²⁰ is selected fro

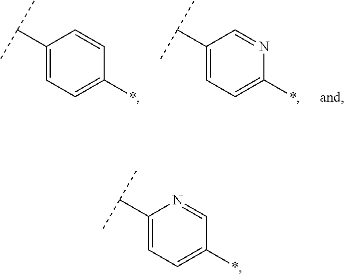

wherein * represents the attachment to L.

In some embodiments, the compound or salt of Formula (II) is a compound represented by the structure of Formula (II-C):

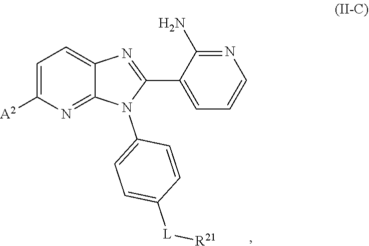

or pharmaceutically acceptable salt thereof, wherein A², L and R²¹ are each defined as in Formula (II).

In some embodiments, for the compound or salt of Formula (II), (II-A), (II-B), or (II-C), L², L³, and L⁴ are absent; and L¹ is selected from C₁₋₆ alkylene optionally substituted with one or more substituents independently selected from halogen, —OR¹⁴, —N(R¹⁴)₂, and ═O. In some embodiments, L², L³, and L⁴ are absent; and L¹ is selected from C₁₋₆ alkylene optionally substituted with one or more substituents independently selected from halogen, —OR¹⁴, —N(R¹⁴)₂, and ═O; and R¹⁴ is selected from hydrogen.

In some embodiments, for the compound or salt of Formula (II), (II-A), (II-B), or (II-C), L², L³, and L⁴ are absent; and L¹ is selected from C₁₋₃ alkylene optionally substituted with ═O.

In some embodiments, for the compound or salt of Formula (II), (II-A), (II-B), or (II-C), L², L³, and L⁴ are absent; and L¹ is selected from

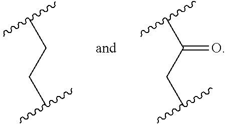

In some embodiments, for the compound or salt of Formula (II), (II-A), (II-B), or (II-C), L³ and L⁴ are absent; and each of L¹ and L² are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—; and
C₁₋₄ alkylene, C₂₋₄ alkenylene, C₂₋₄ alkynylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN.

In some embodiments, for the compound or salt of Formula (II), (II-A), (II-B), or (II-C), L³ and L⁴ are absent; and each of L¹ and L² are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—; and
C₁₋₄ alkylene, C₂₋₄ alkenylene, C₂₋₄ alkynylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN; and
R¹³ and R¹⁴ are each independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O.

In some embodiments, for the compound or salt of Formula (II), (II-A), (II-B), or (II-C), L³ and L⁴ are absent; and each of L¹ and L² are independently selected from:
—N(R¹⁴)—, —N(R¹⁴)C(O)—, and —N(R¹⁴)S(O)₂—; and
C₁₋₄ alkylene, C₂₋₄ alkynylene, and 3- to 6-membered heterocyclene, any one of which is optionally substituted with one or more substituents independently selected from halogen, —OR¹³, —SR¹³, —N(R¹³)₂, and —CN.

In some embodiments, for the compound or salt of Formula (II), (II-A), (II-B), or (II-C), L³ and L⁴ are absent; and each of L¹ and L² are independently selected from:
—N(R¹⁴)C(O)— and —N(R¹⁴)S(O)₂—; and
C₁₋₄ alkylene, C₂₋₄ alkynylene, and 3- to 6-membered heterocyclene, any one of which is optionally substituted with one or more substituents independently selected from halogen and —OR¹³.

In some embodiments, for the compound or salt of Formula (II), (II-A), (II-B), or (II-C), L³ and L⁴ are absent; and each of L¹ and L² are independently selected from:
—N(R¹⁴)C(O)— and —N(R¹⁴)S(O)₂—; and
C₁₋₄ alkylene, C₂₋₄ alkynylene, and 3- to 6-membered heterocyclene.

In some embodiments, for the compound or salt of Formula (II), (II-A), (II-B), or (II-C), L³ and L⁴ are absent; and each of L¹ and L² are independently selected from:
—N(R¹⁴)C(O)— and —N(R¹⁴)S(O)₂—; and
C₁₋₄ alkylene, C₂₋₄ alkynylene, and piperidinylene.

In some embodiments, for the compound or salt of Formula (II), (II-A), (II-B), or (II-C), L³ and L⁴ are absent; and each of L¹ and L² are independently selected from:
—N(R¹⁴)C(O)— and —N(R¹⁴)S(O)₂—; and
C₁₋₄ alkylene, C₂₋₄ alkynylene, and piperidinylene; and
R¹⁴ is independently selected at each occurrence from hydrogen abd C₁₋₄ alkyl.

In some embodiments, for the compound or salt of Formula (II), (II-A), (II-B), or (II-C), L³ and L⁴ are absent; and each of L¹ and L² are independently selected from C₁₋₄ alkylene, C₂₋₄ alkynylene, and 3- to 6-membered heterocyclene, any one of which is optionally substituted with one or more substituents independently selected from halogen, —OR¹³, —SR¹³, —N(R¹³)₂, and —CN. In some embodiments, L³ and L⁴ are absent; and each of L¹ and L² are independently selected from C₁₋₄ alkylene, C₂₋₄ alkynylene, and 3- to 6-membered heterocyclene, any one of which is optionally substituted with one or more substituents independently selected from halogen, —OR¹³, —SR¹³, —N(R¹³)₂, and —CN; and R¹³ is selected from each occurrence from hydrogen and C₁₋₄ alkyl. In some embodiments, L³ and L⁴ are absent; and each of L¹ and L² are independently selected from C₁₋₄ alkylene, and piperidinylene.

In some embodiments, for the compound or salt of Formula (II), (II-A), (II-B), or (II-C), L³ and L⁴ are absent; and
L¹ is selected from —O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, and —N(R¹⁴)N(R¹⁴)—; and
L² is selected from C₁₋₄ alkylene, C₂₋₄ alkenylene, C₂₋₄ alkynylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN.

In some embodiments, for the compound or salt of Formula (II), (II-A), (II-B), or (II-C), L³ and L⁴ are absent; and
L¹ is selected from —O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, and —N(R¹⁴)N(R¹⁴)—; and
L² is selected from C₁₋₄ alkylene, C₂₋₄ alkenylene, C₂₋₄ alkynylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN; and
R¹³ and R¹⁴ are each independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O.

In some embodiments, for the compound or salt of Formula (II), (II-A), (II-B), or (II-C), L³ and L⁴ are absent; and
L¹ is selected from —N(R¹⁴)—, —N(R¹⁴)C(O)—, and —N(R¹⁴)S(O)₂—; and
L² is selected from C₁₋₄ alkylene, C₂₋₄ alkynylene, and 3- to 6-membered heterocyclene, any one of which is optionally substituted with one or more substituents independently selected from halogen, —OR¹³, —SR¹³, —N(R¹³)₂, and —CN.

In some embodiments, for the compound or salt of Formula (II), (II-A), (II-B), or (II-C), L³ and L⁴ are absent; and
L¹ is selected from —N(R¹⁴)—, —N(R¹⁴)C(O)—, and —N(R¹⁴)S(O)₂—; and
L² is selected from C₁₋₄ alkylene, C₂₋₄ alkynylene, and 3- to 6-membered heterocyclene, any one of which is optionally substituted with one or more substituents independently selected from halogen, —OR¹³, —SR¹³, —N(R¹³)₂, and —CN; and
R¹³ is independently selected at each occurrence from hydrogen and C₁₋₄ alkyl.

In some embodiments, for the compound or salt of Formula (II), (II-A), (II-B), or (II-C), L³ and L⁴ are absent; and
L¹ is selected from —N(R¹⁴)C(O)— and —N(R¹⁴)S(O)₂—; and
L² is selected from C₁₋₄ alkylene, C₂₋₄ alkynylene, and 3- to 6-membered heterocyclene.

In some embodiments, for the compound or salt of Formula (II), (II-A), (II-B), or (II-C), L³ and L⁴ are absent; and
L¹ is selected from —N(R¹⁴)C(O)— and —N(R¹⁴)S(O)₂—; and
L² is selected from C₁₋₄ alkylene, C₂₋₄ alkynylene, and piperidinylene.

In some embodiments, for the compound or salt of Formula (II), (II-A), (II-B), or (II-C), L³ and L⁴ are absent; and
L¹ is selected from —N(R¹⁴)C(O)— and —N(R¹⁴)S(O)₂—; and
L² is selected from C₁₋₄ alkylene, C₂₋₄ alkynylene, and piperidinylene; and
R¹⁴ is independently selected at each occurrence from hydrogen and C₁₋₄ alkyl.

In some embodiments, for the compound or salt of Formula (II), (II-A), (II-B), or (II-C), L³ and L⁴ are absent; and
L¹ is C₁₋₄ alkylene; and
L² is selected from 3- to 6-membered heterocyclene.

In some embodiments, for the compound or salt of Formula (II), (II-A), (II-B), or (II-C), L³ and L⁴ are absent; and
L¹ is C₁₋₄ alkylene; and
L² is piperidinylene.

In some embodiments, for the compound or salt of Formula (II), (II-A), (II-B), or (II-C), L is selected from:

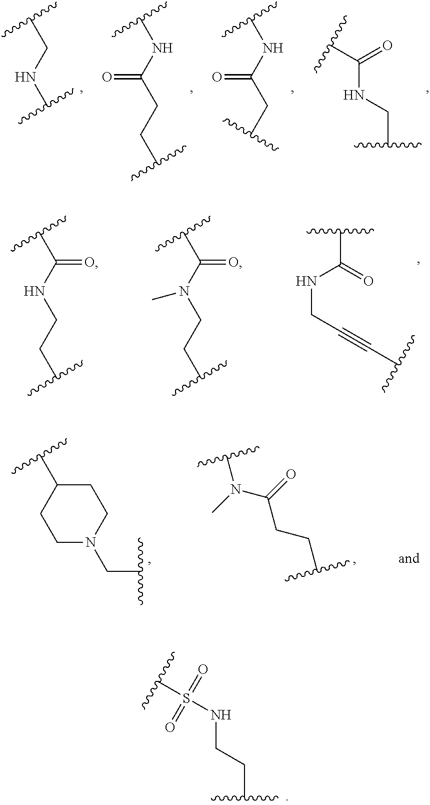

In some embodiments, for the compound or salt of Formula (II), (II-A), (II-B), or (II-C), L is

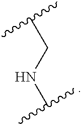 [SMILES: *CN*]

In some embodiments, for the compound or salt of Formula (II), (II-A), (II-B3), or (II-C), L is

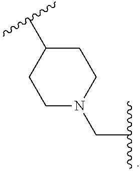 [SMILES: *CN1CCC(*)CC1]

In some embodiments, for the compound or salt of Formula (II), (II-A), (II-B), or (II-C), L is:

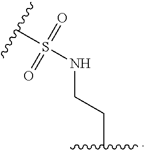 [SMILES: *CCNS(*)(=O)=O]

In some embodiments, for the compound or salt of Formula (II), (II-A), (II-B), or (II-C), L is selected from:

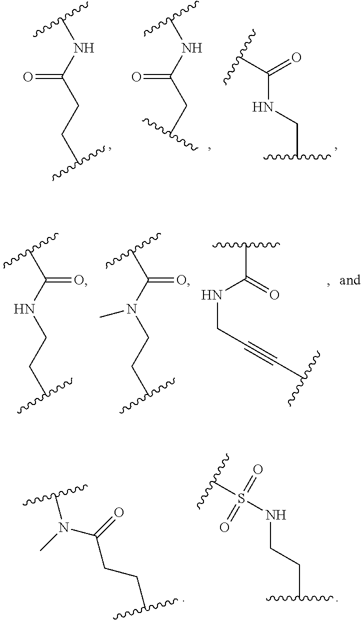

In some embodiments, for the compound or salt of Formula (II), (II-A), (II-B), or (II-C), L is selected from:

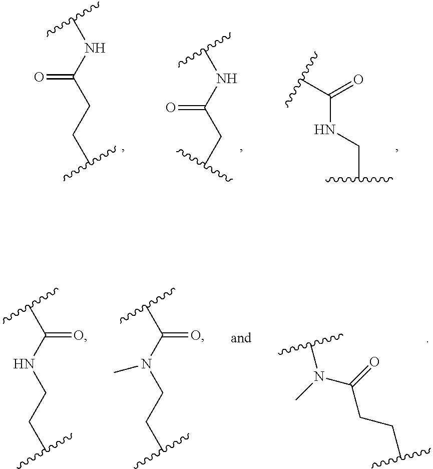

In some embodiments, for the compound or salt of Formula (II), (II-A), (II-B), or (II-C), L⁴ is absent; and each of L¹, L², and L³ are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—; and
C₁₋₄ alkylene, C₂₋₄ alkenylene, C₂₋₄ alkynylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN.

In some embodiments, for the compound or salt of Formula (II), (II-A), (II-B), or (II-C), L⁴ is absent; and each of L¹, L², and L³ are independently selected from:
—N(R¹⁴)— and N(R¹⁴)C(O)—; and
C₁₋₄ alkylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN; and
R¹³ and R¹⁴ are each independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O.

In some embodiments, for the compound or salt of Formula (II), (II-A), (II-B), or (II-C), L⁴ is absent; and each of L¹, L², and L³ are independently selected from:
—O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—; and
C₁₋₄ alkylene, C₂₋₄ alkenylene, C₂₋₄ alkynylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN.

In some embodiments, for the compound or salt of Formula (II), (II-A), (II-B), or (II-C), L⁴ is absent; and each of L¹, L², and L³ are independently selected from:
—N(R¹⁴)— and N(R¹⁴)C(O)—; and
C₁₋₄ alkylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN.

In some embodiments, for the compound or salt of Formula (II), (II-A), (II-B), or (II-C), L⁴ is absent; and each of L¹, L², and L³ are independently selected from:
—N(R¹⁴)— and N(R¹⁴)C(O)—; and
C₁₋₄ alkylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN; and
R¹³ is independently selected at each occurrence from hydrogen and C₁₋₄ alkyl.

In some embodiments, for the compound or salt of Formula (II), (II-A), (II-B), or (II-C), L⁴ is absent; and each of L¹, L², and L³ are independently selected from:
—N(R¹⁴)— and N(R¹⁴)C(O)—; and
C₁₋₄ alkylene, C₃₋₆ carbocyclene, and 3- to 6-membered heterocyclene, any one of which is optionally substituted with one or more substituents independently selected from halogen and —OR¹³.

In some embodiments, for the compound or salt of Formula (II), (II-A), (II-B), or (II-C), L⁴ is absent; and each of L¹, L², and L³ are independently selected from:
—N(R¹⁴)— and N(R¹⁴)C(O)—; and
C₁₋₄ alkylene, cyclopropylene, cyclobutylene, azetidinylene, and pyridinylene, any one of which is optionally substituted with one or more substituents independently selected from halogen and —OR¹³; and
R¹⁴ is independently selected at each occurrence from hydrogen and C₁₋₄ alkyl.

In some embodiments, for the compound or salt of Formula (II), (II-A), (II-B), or (II-C), L⁴ is absent; and
L² is selected
   from —O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—; and
L¹ and L³ are independently selected from C₁₋₄ alkylene, C₂₋₄ alkenylene, C₂₋₄ alkynylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN.

In some embodiments, for the compound or salt of Formula (II), (II-A), (II-B), or (II-C), L⁴ is absent; and
L² is selected
   from —O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—; and
L¹ and L³ are independently selected from C₁₋₄ alkylene, C₂₋₄ alkenylene, C₂₋₄ alkynylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN; and
R¹³ and R¹⁴ are each independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O.

In some embodiments, for the compound or salt of Formula (II), (II-A), (II-B), or (II-C), L⁴ is absent; and
L² is selected from —N(R¹⁴)— and —N(R¹⁴)C(O)—; and
L¹ and L³ are independently selected from C₁₋₄ alkylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN.

In some embodiments, for the compound or salt of Formula (II), (II-A), (II-B), or (II-C), L⁴ is absent; and
L² is selected from —N(R¹⁴)— and —N(R¹⁴)C(O)—; and
L¹ and L³ are independently selected from C₁₋₄ alkylene, C₃₋₆ carbocyclene, and 3- to 6-membered heterocyclene, any one of which is optionally substituted with one or more substituents independently selected from halogen, —OR¹³, —SR¹³, —N(R¹³)₂, ═O, ═S, and —CN; and
R¹³ is independently selected at each occurrence from hydrogen and C₁₋₄ alkyl.

In some embodiments, for the compound or salt of Formula (II), (II-A), (II-B), or (II-C), L⁴ is absent; and
L² is selected from —N(R¹⁴)— and N(R¹⁴)C(O)—; and
L¹ and L³ are independently selected from C₁₋₄ alkylene, C₃₋₆ carbocyclene, and 3- to 6-membered heterocyclene, any one of which is optionally substituted with one or more substituents independently selected from halogen and —OR¹³.

In some embodiments, for the compound or salt of Formula (II), (II-A), (II-B), or (II-C), L⁴ is absent; and
L² is selected from —N(R¹⁴)— and N(R¹⁴)C(O)—; and
L¹ and L³ are independently selected from C₁₋₄ alkylene, C₃₋₆ carbocyclene, and 3- to 6-membered heterocyclene, any one of which is optionally substituted with one or more substituents independently selected from halogen and —OR¹³.

In some embodiments, for the compound or salt of Formula (II), (II-A), (II-B), or (II-C), L⁴ is absent; and
L² is selected from —N(R¹⁴)— and —N(R¹⁴)C(O)—; and
L¹ and L³ are independently selected from C₁₋₄ alkylene, cyclopropylene, cyclobutylene, azetidinylene, and pyridinylene, any one of which is optionally substituted with one or more substituents independently selected from halogen and —OR¹³; and
R¹⁴ is independently selected at each occurrence from hydrogen and C₁₋₄ alkyl.

In some embodiments, for the compound or salt of Formula (II), (II-A), (II-B), or (II-C), L is selected from:

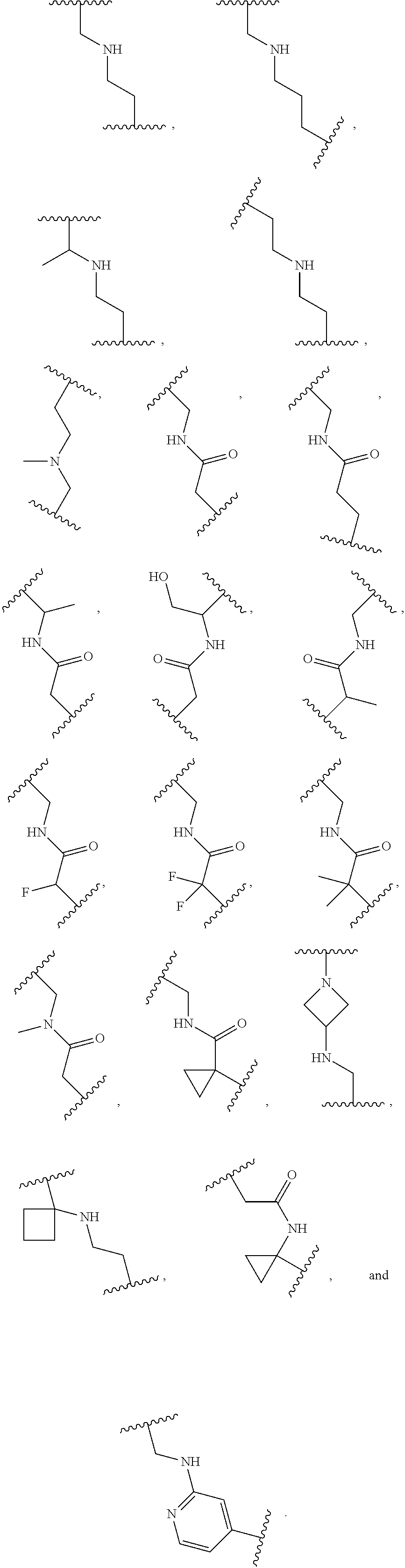

and In some embodiments, for the compound or salt of Formula (II), (II-A), (II-B), or (II-C), L is selected from:

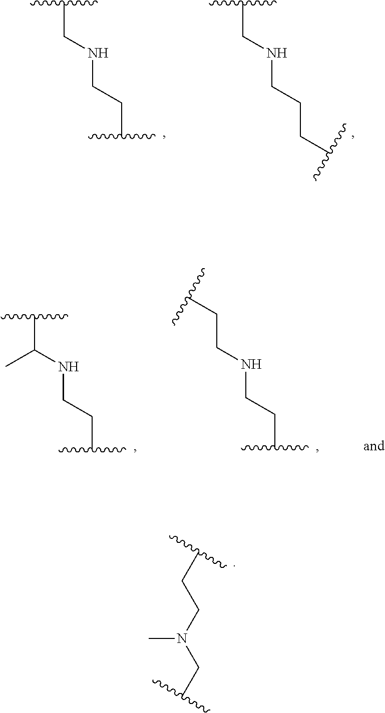

In some embodiments, for the compound or salt of Formula (II), (II-A), (II-B), or (II-C), L is selected from:

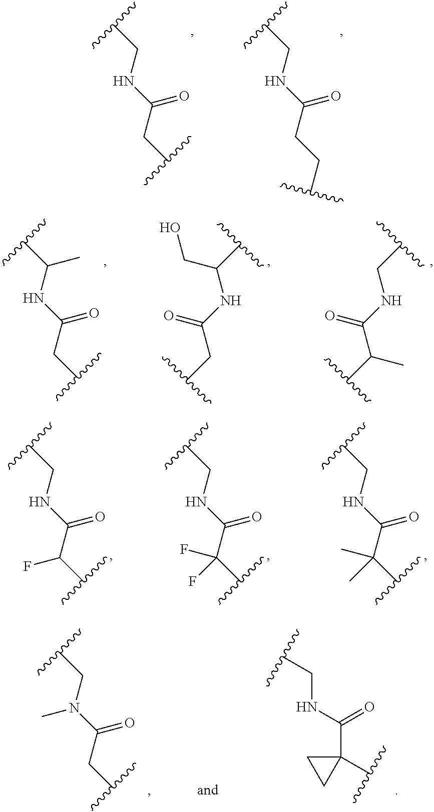

In some embodiments, for the compound or salt of Formula (II), (II-A), (II-B), or (II-C), L is selected from:

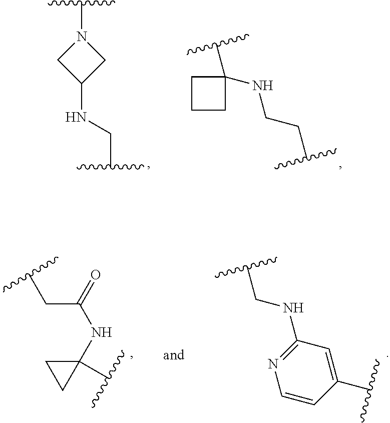 [SMILES: *CNC1CN(*)C1 ; *CCNC1(*)CCC1 ; *CC(=O)NC1(*)CC1 ; *CNc1cc(*)ccn1]

In some embodiments, for the compound of salt of Formula (II), (II-A), (II-B), and (II), L is selected from:

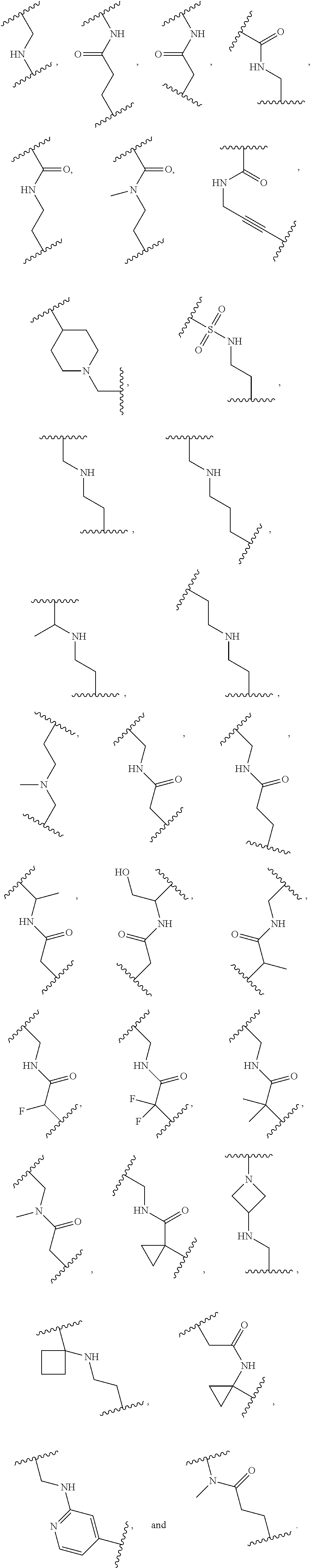

In some embodiments, for the compound or salt of Formula (II), (II-A), (II-B), or (II-C), R²¹ is selected from 5- to 6-membered heterocycle and C₃₋₆ carbocycle, any one of which is substituted with —C(O)H, wherein R²¹ is further optionally substituted with one or more substituents independently selected from halogen, C₁₋₄ alkyl, C₁₋₄ haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN. In some embodiments, R²¹ is selected from 5- to 6-membered heterocycle and C₃₋₆ carbocycle, any one of which is substituted with —C(O)H, wherein R²¹ is further optionally substituted with one or more substituents independently selected from halogen, C₁₋₄ alkyl, C₁₋₄ haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN; and R¹⁵ is independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O.

In some embodiments, for the compound or salt of Formula (II), (II-A), (II-B), or (II-C), R²¹ is selected from 5- to 6-membered heterocycle and C₃₋₆ carbocycle, any one of which is substituted with —C(O)H, wherein R²¹ is further optionally substituted with one or more substituents independently selected from halogen, C₁₋₄ alkyl, C₁₋₄ haloalkyl, —OR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —N(R¹⁵) C(O)N(R¹⁵)₂, and —CN.

In some embodiments, for the compound or salt of Formula (II), (II-A), (II-B), or (II-C), R²¹ is selected from 5- to 6-membered heterocycle and C₃₋₆ carbocycle, any one of which is substituted with —C(O)H, wherein R²¹ is further optionally substituted with one or more substituents independently selected from halogen, C₁₋₄ alkyl, C₁₋₄ haloalkyl, —OR¹⁵, —N(R¹⁵)C(O)R¹⁵, and —N(R¹⁵)C(O)N(R¹⁵)₂. In some embodiments, R²¹ is selected from 5- to 6-membered heterocycle and C₃₋₆ carbocycle, any one of which is substituted with —C(O)H, wherein R²¹ is further optionally substituted with one or more substituents independently selected from halogen, C₁₋₄ alkyl, C₁₋₄ haloalkyl, —OR¹⁵, —N(R¹⁵)C(O)R¹⁵, and —N(R¹⁵)C(O)N(R¹⁵)₂; and R¹⁵ is independently selected at each occurrence from hydrogen and C₁₋₄ alkyl.

In some embodiments, for the compound or salt of Formula (II), (II-A), (II-B), or (II-C), R²¹ is selected from 5- to 6-membered heterocycle and C₃₋₆ carbocycle, any one of which is substituted with —C(O)H, wherein R²¹ is further optionally substituted with one or more substituents independently selected from fluoro, chloro, bromo, hydroxyl, methyl,

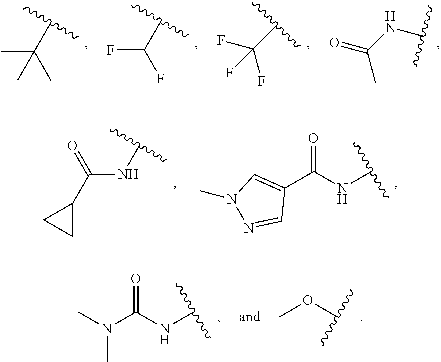

In some embodiments, for the compound or salt of Formula (II), (II-A), (II-B), or (II-C), R²¹ is selected from 6-membered heteroarylene and phenylene, any one of which is substituted with —C(O)H, wherein R²¹ is further optionally substituted with one or more substituents independently selected from halogen, C₁₋₄ alkyl, C₁₋₄ haloalkyl, —OR⁵, —SR⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, and —CN.

In some embodiments, for the compound or salt of Formula (II), (II-A), (II-B), or (II-C), R²¹ is selected from 6-membered heteroarylene and phenylene, any one of which is substituted with —C(O)H, wherein R²¹ is further optionally substituted with one or more substituents independently selected from halogen, C₁₋₄ alkyl, C₁₋₄ haloalkyl, —OR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —N(R¹⁵)C(O)N(R¹⁵)₂, and —CN.

In some embodiments, for the compound or salt of Formula (II), (II-A), (II-B), or (II-C), R²¹ is selected from 6-membered heteroarylene and phenylene, any one of which is substituted with —C(O)H, wherein R²¹ is further optionally substituted with one or more substituents independently selected from halogen, C₁₋₄ alkyl, C₁₋₄ haloalkyl, —OR¹⁵, —N(R¹⁵)C(O)R¹⁵, and —N(R¹⁵)C(O)N(R¹⁵)₂.

In some embodiments, for the compound or salt of Formula (II), (II-A), (II-B), or (II-C), R²¹ is selected from 6-membered heteroarylene and phenylene, any one of which is substituted with —C(O)H, wherein R²¹ is further optionally substituted with one or more substituents independently selected from fluoro, chloro, bromo, hydroxyl, methyl,

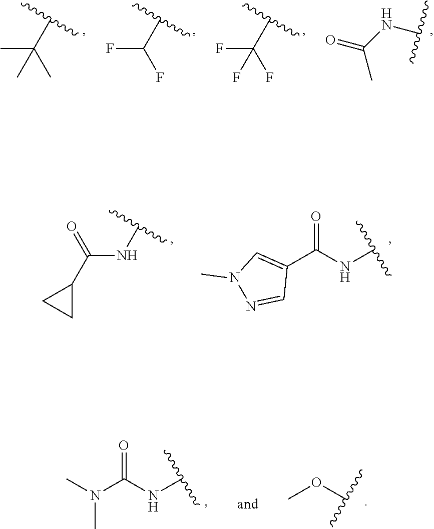

In some embodiments, for the compound or salt of Formula (II), (II-A), (II-B), or (II-C), R²¹ is selected from pyridinylene and phenylene, any one of which is substituted with —C(O)H, wherein R²¹ is further optionally substituted with one or more substituents independently selected from halogen, C₁₋₄ alkyl, C₁₋₄ haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, and —CN. In some embodiments, R²¹ is selected from pyridinylene and phenylene, any one of which is substituted with —C(O)H, wherein R²¹ is further optionally substituted with one or more substituents independently selected from halogen, C₁₋₄ alkyl, C₁₋₄ haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹¹, —NO₂, and —CN; and R¹⁵ is independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O.

In some embodiments, for the compound or salt of Formula (II), (II-A), (II-B), or (II-C), R²¹ is selected from pyridinylene and phenylene, any one of which is substituted with —C(O)H, wherein R²¹ is further optionally substituted with one or more substituents independently selected from halogen, C₁₋₄ alkyl, C₁₋₄ haloalkyl, —OR⁵, —N(R¹¹)₂, —C(O)R¹⁵, —C(O)N(R¹⁵)₂, —N(R¹⁵)C(O)R¹⁵, —N(R¹⁵)S(O)₂R¹⁵, —N(R¹⁵)C(O)N(R¹⁵)₂, and —CN.

In some embodiments, for the compound or salt of Formula (II), (II-A), (II-B), or (II-C), R²¹ is selected from pyridinylene and phenylene, any one of which is substituted with —C(O)H, wherein R²¹ is further optionally substituted with one or more substituents independently selected from halogen, C₁₋₄ alkyl, C₁₋₄ haloalkyl, —OR¹⁵, —N(R¹⁵)C(O)R^{1′} and —N(R¹⁵)C(O)N(R¹⁵)₂. In some embodiments, R²¹ is selected from pyridinylene and phenylene, any one of which is substituted with —C(O)H, wherein R²¹ is further optionally substituted with one or more substituents independently selected from halogen, C₁₋₄ alkyl, C₁₋₄ haloalkyl, —OR¹⁵, —N(R¹⁵)C(O)R¹⁵, and —N(R¹⁵)C(O)N(R¹⁵)₂; and R¹¹ is independently selected at each occurrence from hydrogen and C₁₋₄ alkyl.

In some embodiments, for the compound or salt of Formula (II), (II-A), (II-B), or (II-C), R²¹ is selected from pyridinylene and phenylene, any one of which is substituted with —C(O)H, wherein R²¹ is further optionally substituted with one or more substituents independently selected from fluoro, chloro, bromo, hydroxyl, methyl,

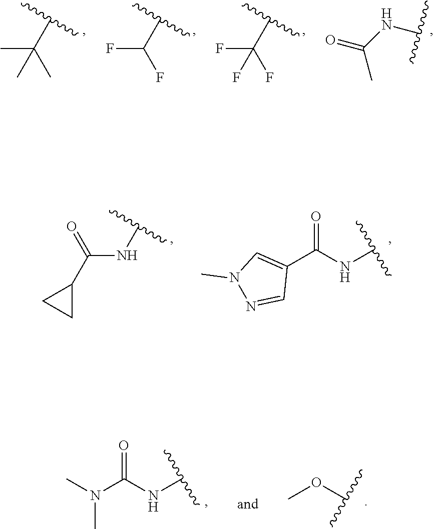

In some embodiments, for the compound or salt of Formula (II), (II-A), (II-B), or (II-C), R²¹ is selected from

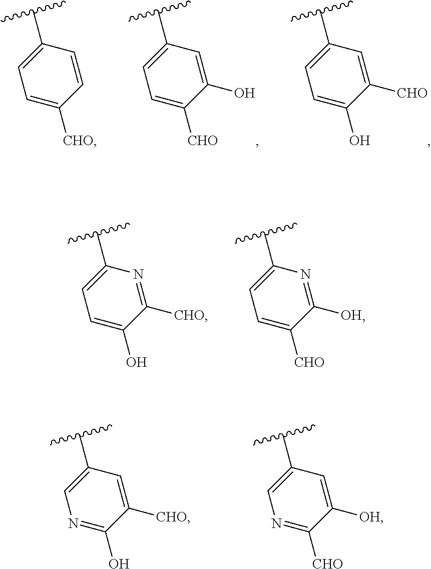

further optionally substituted with one or more substituents independently selected from halogen, C₁₋₄ alkyl, C₁₋₄ haloalkyl, —OR^{D}, —SR¹¹, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, and —CN.

In some embodiments, for the compound or salt of Formula (II), (II-A), (II-B), or (II-C), R²¹ is selected from

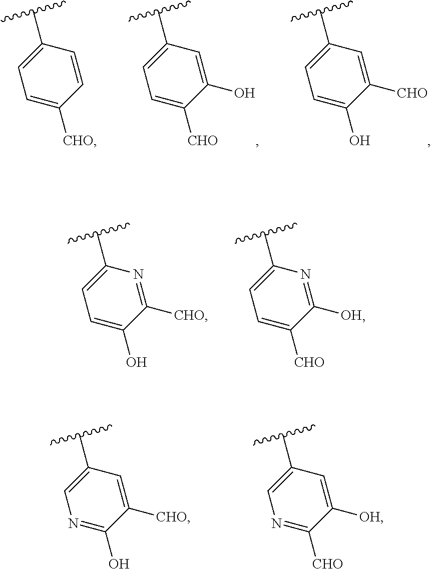

further optionally substituted with one or more substituents independently selected from halogen, C₁₋₄ alkyl, C₁₋₄ haloalkyl, —OR¹⁵, —N(R¹⁵)₂, —C(O)R¹⁵, —C(O)N(R¹⁵)₂, —N(R¹⁵)C(O)R¹⁵, —N(R¹⁵)S(O)₂R¹⁵, —N(R¹⁵)C(O)N(R¹⁵)₂, and —CN.

In some embodiments, for the compound or salt of Formula (II), (II-A), (II-B), or (II-C), R²¹ is selected from

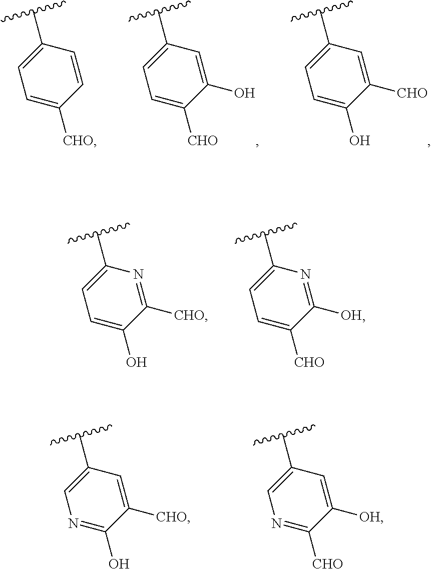

further optionally substituted with one or more substituents independently selected from halogen, C₁₋₄ alkyl, C₁₋₄ haloalkyl, —OR¹⁵, —N(R¹⁵)C(O)R¹⁵, and —N(R¹⁵)C(O)N(R¹⁵)₂. In some embodiments, R²¹ is selected from

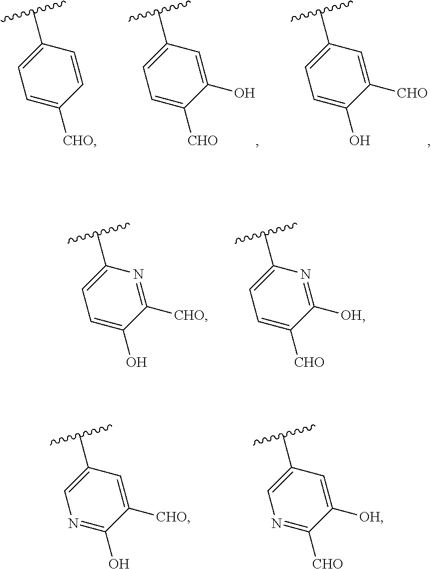

further optionally substituted with one or more substituents independently selected from halogen, C₁₋₄ alkyl, C₁₋₄ haloalkyl, —OR¹⁵, —N(R¹⁵)C(O)R¹⁵, and —N(R¹⁵)C(O)N(R¹⁵)₂; and R¹⁵ is independently selected at each occurrence from hydrogen and C₁₋₄ alkyl.

In some embodiments, for the compound or salt of Formula (II), (II-A), (II-B), or (II-C), R²¹ is selected from

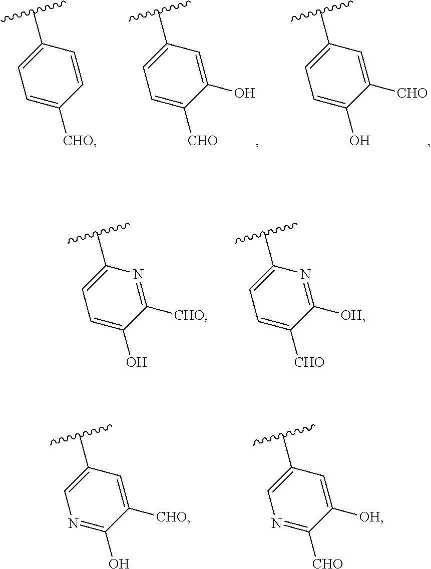

further optionally substituted with one or more substituents independently selected from fluoro, chloro, bromo, methyl,

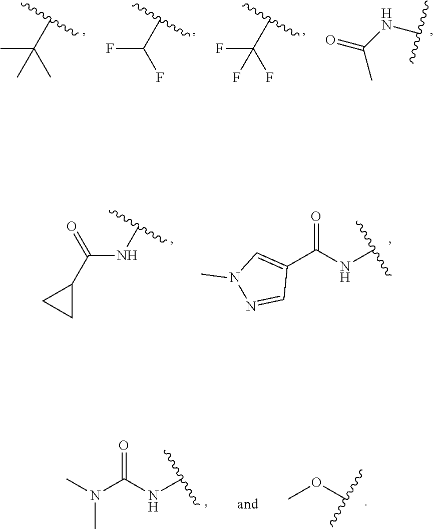

In some embodiments, for the compound or salt of Formula (II), (II-A), (II-B), or (II-C), R²¹ is selected from:

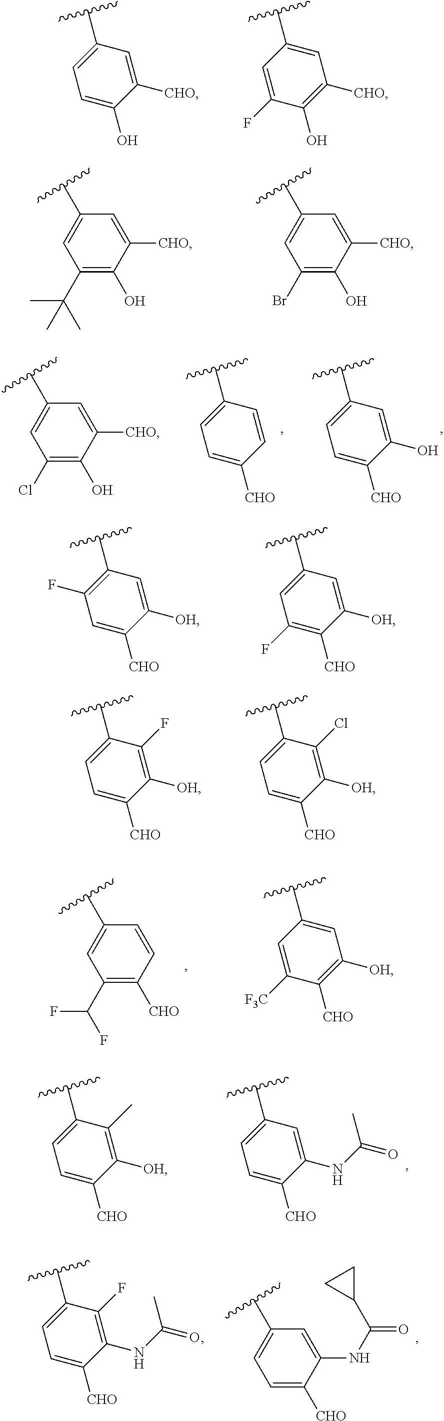

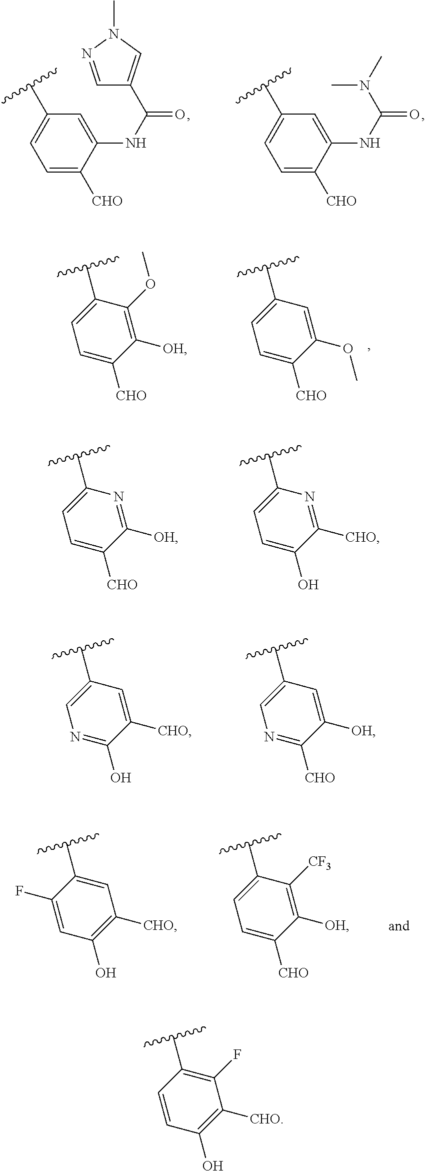

In some embodiments, for the compound or salt of Formula (II), (II-A), (II-B), or (II-C), R²¹ is selected from:

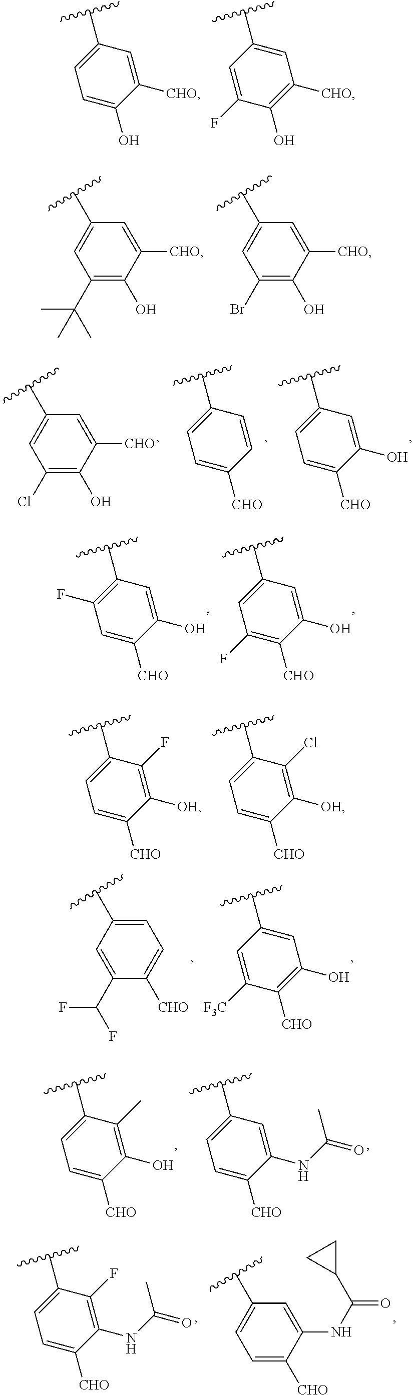

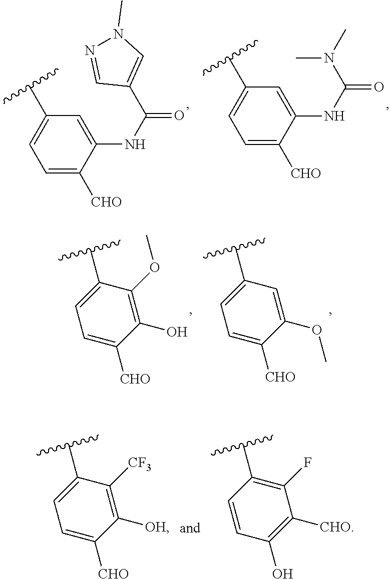 [SMILES: *c1ccc(C=O)c(NC(=O)c2cnn(C)c2)c1 ; *c1ccc(C=O)c(NC(=O)N(C)C)c1 ; *c1ccc(C=O)c(O)c1OC ; *c1ccc(C=O)c(OC)c1 ; *c1ccc(C=O)c(O)c1C(F)(F)F ; *c1ccc(O)c(C=O)c1F]

In some embodiments, for the compound or salt of Formula (II), (II-A), (II-B), or (II-C), R²¹ is selected from:

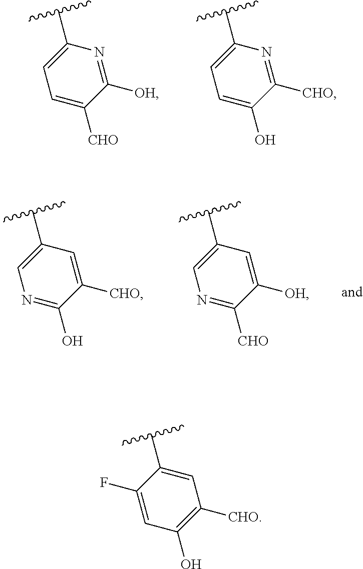 [SMILES: *c1ccc(C=O)c(O)n1 ; *c1ccc(O)c(C=O)n1 ; *c1cnc(O)c(C=O)c1 ; *c1cnc(C=O)c(O)c1 ; *c1cc(C=O)c(O)cc1F]

In some embodiments, for the compound or salt of Formula (II), (II-A), (II-B), or (II-C), R¹⁰, R¹¹, R¹², R¹³, R¹⁴, and R¹⁵ are each independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O. In some embodiments, R¹⁰, R¹¹, R¹², R¹³, R¹⁴, and R¹⁵ are each independently selected at each occurrence from hydrogen, C₁₋₄ alkyl, C₃₋₈ carbocycle, and 3- to 8-membered heterocycle, wherein the C₃₋₈ carbocycle and 3- to 8-membered heterocycle are each optionally substituted with one or more C₁₋₄ alkyl. In some embodiments, R¹⁰, R, R¹², R¹³, R¹⁴, and R¹⁵ are each independently selected at each occurrence from hydrogen and C₁₋₄ alkyl. In some embodiments, R¹⁰, R, R¹², R¹³, R¹⁴, and R¹⁵ are hydrogen.

In some embodiments, for the compound or salt of Formula (II), (II-A), (II-B), or (II-C), R¹⁰ is independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O. In some embodiments, R¹⁰ is independently selected at each occurrence from hydrogen and C₁₋₄ alkyl. In some embodiments, R¹⁰ is hydrogen.

In some embodiments, for the compound or salt of Formula (II), (II-A), (II-B), or (II-C), R¹¹ is independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O. In some embodiments, R¹¹ is independently selected at each occurrence from hydrogen, C₁₋₄ alkyl, C₃₋₈ carbocycle, and 3- to 8-membered heterocycle, wherein the C₃₋₈ carbocycle and 3- to 8-membered heterocycle are each optionally substituted with one or more C₁₋₄ alkyl. In some embodiments, R¹¹ is independently selected from: hydrogen, C₁₋₃ alkyl, C₁₋₃ haloalkyl, and C₃₋₄ carbocycle In some embodiments, R¹¹ is independently selected at each occurrence from hydrogen and C₁₋₄ alkyl. In some embodiments, R¹¹ is hydrogen.

In some embodiments, for the compound or salt of Formula (II), (II-A), (II-B), or (II-C), R¹² is independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O. In some embodiments, R¹² is independently selected at each occurrence from hydrogen and C₁₋₄ alkyl. In some embodiments, R¹² is hydrogen.

In some embodiments, for the compound or salt of Formula (II), (II-A), (II-B), or (II-C), R¹³ is independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O. In some embodiments, R¹³ is independently selected at each occurrence from hydrogen and C₁₋₄ alkyl. In some embodiments, R¹³ is hydrogen.

In some embodiments, for the compound or salt of Formula (II), (II-A), (II-B), or (II-C), R¹⁴ is independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O. In some embodiments, R¹⁴ is independently selected at each occurrence from hydrogen and C₁₋₄ alkyl. In some embodiments, R¹⁴ is hydrogen.

In some embodiments, for the compound or salt of Formula (II), (II-A), (II-B), or (II-C), R¹⁵ is independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O. In some embodiments, R¹⁵ is independently selected at each occurrence from hydrogen and C₁₋₄ alkyl. In some embodiments, R¹⁵ is hydrogen.

In some embodiments, the compounds of Formula (I) and (II) are a compound of Table 1.

TABLE 1
Chemical structures of selected compounds

| Cmpd. | |
|---|---|
| No. | Structure |
| 1 | 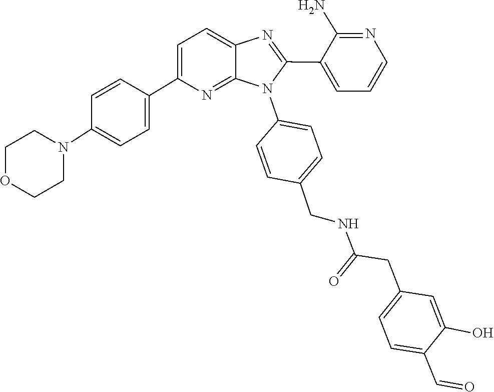 [SMILES: Nc1ncccc1-c1nc2ccc(-c3ccc(N4CCOCC4)cc3)nc2n1-c1ccc(CNC(=O)Cc2ccc(C=O)c(O)c2)cc1] |
| 2 | 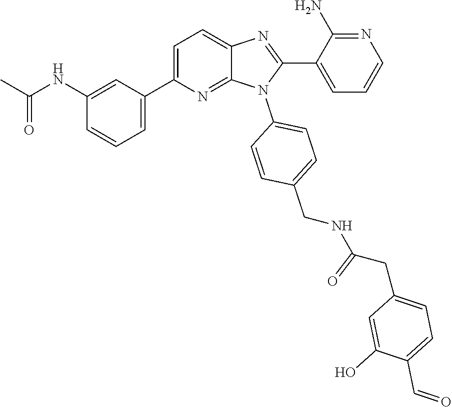 [SMILES: CC(=O)Nc1cccc(-c2ccc3nc(-c4cccnc4N)n(-c4ccc(CNC(=O)Cc5ccc(C=O)c(O)c5)cc4)c3n2)c1] |
| 3 | 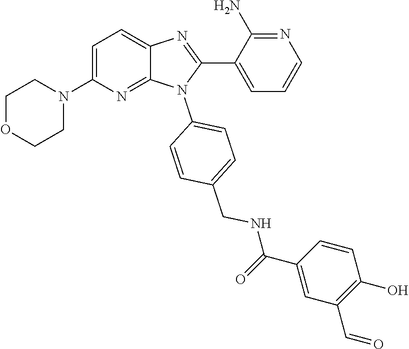 [SMILES: Nc1ncccc1-c1nc2ccc(N3CCOCC3)nc2n1-c1ccc(CNC(=O)c2ccc(O)c(C=O)c2)cc1] |
| 4 | 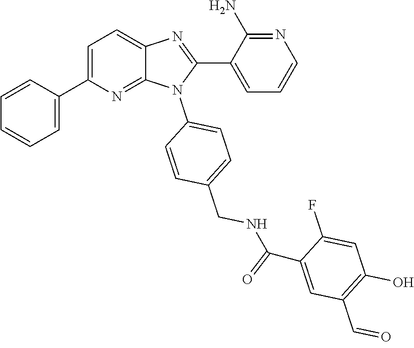 |
| 5 | 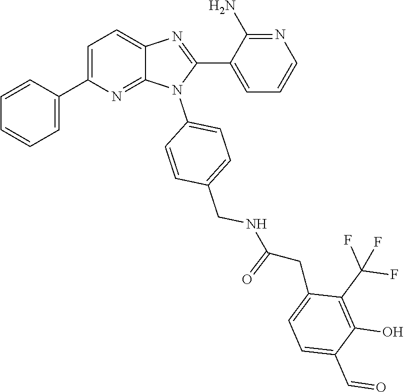 |
| 6 | 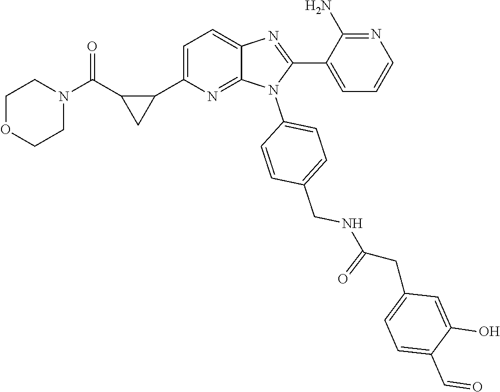 |
| 7 | 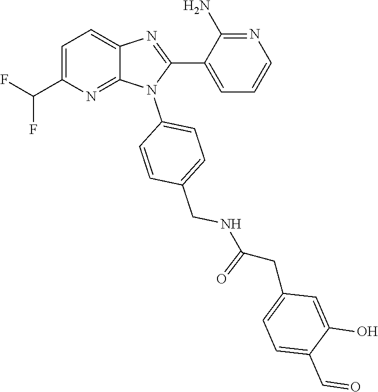 |
| 8 | 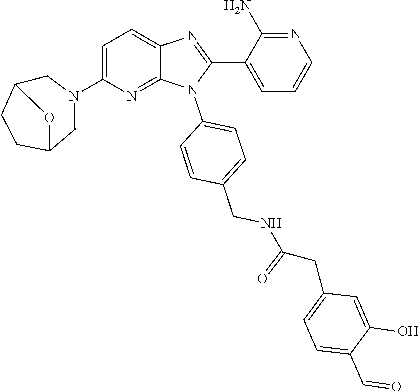 |
| 9 | 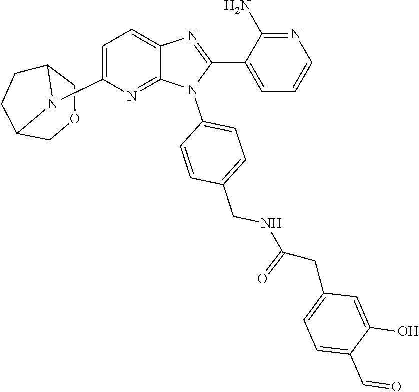 |
| 10 | 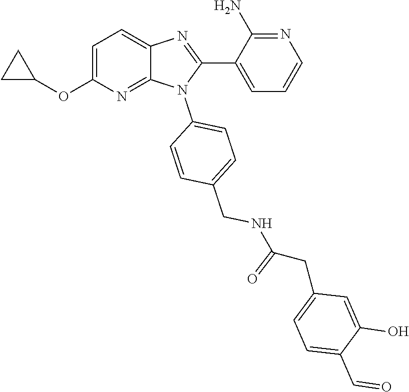 |
| 11 | 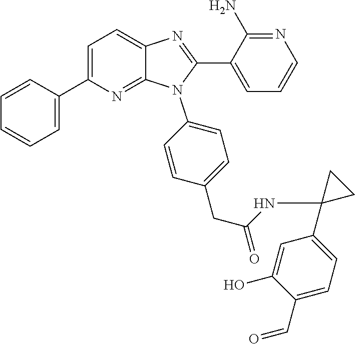 [SMILES: Nc1ncccc1-c1nc2ccc(-c3ccccc3)nc2n1-c1ccc(CC(=O)NC2(c3ccc(C=O)c(O)c3)CC2)cc1] |
| 12 | 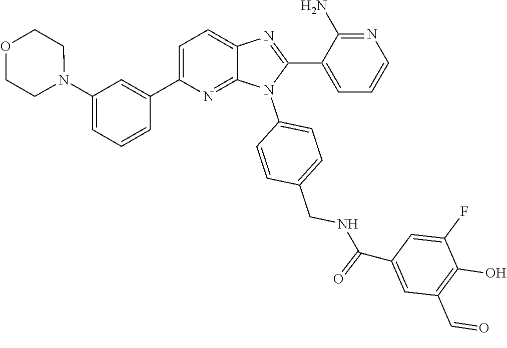 |
| 13 | 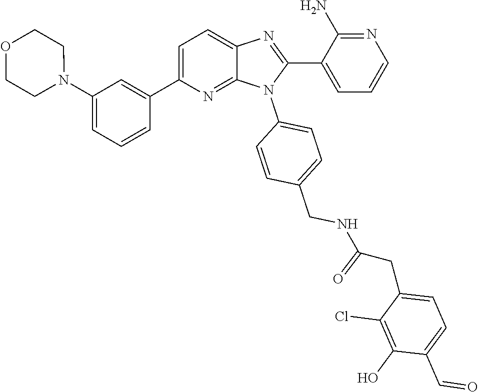 |
| 14 | 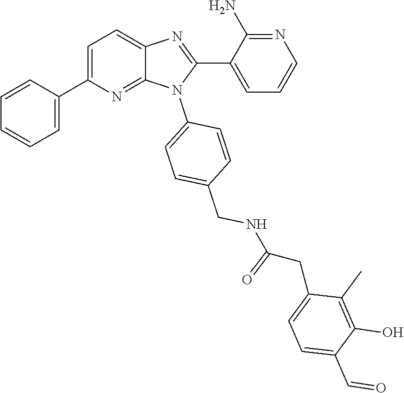 |
| 15 | 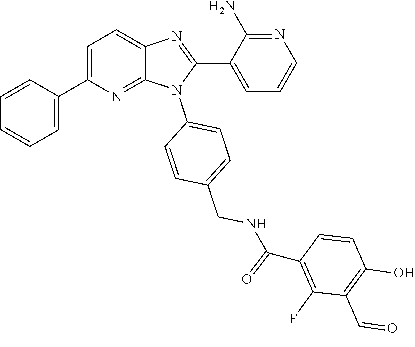 |
| 16 | 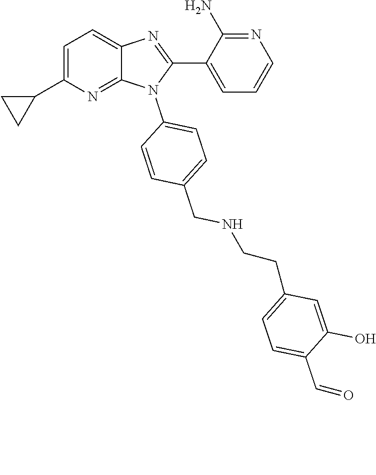 |
| 17 | 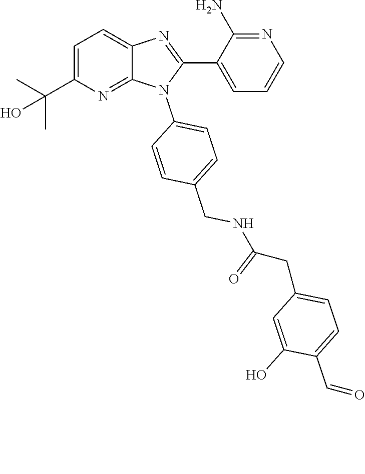 |
| 18 | 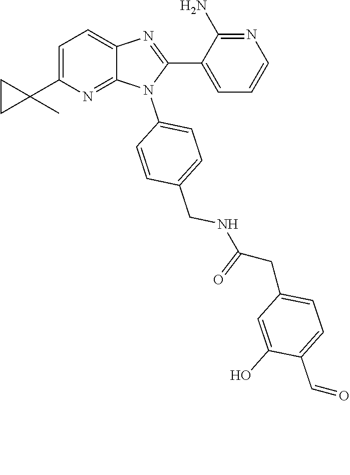 |
| 19 | 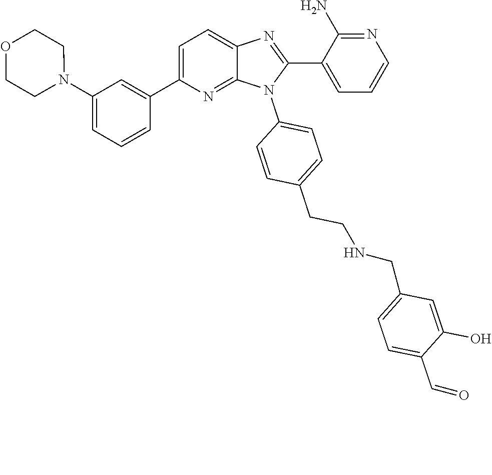 |
| 20 | 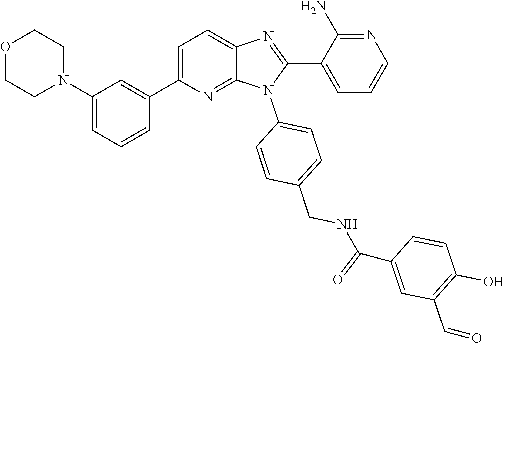 |
| 21 | 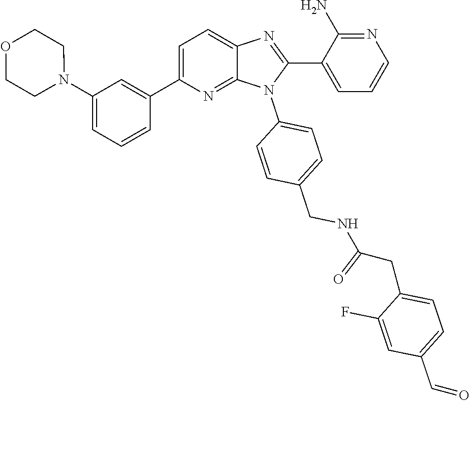 |
| 22 | 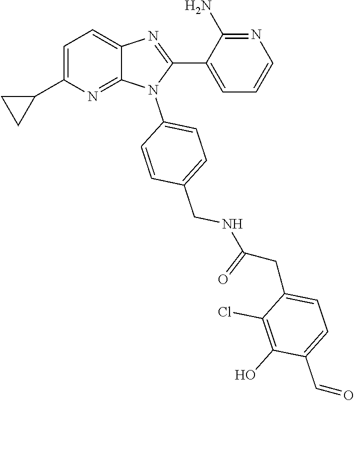 |
| 23 | 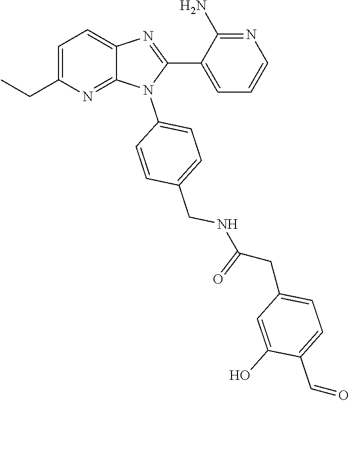 |
| 24 | 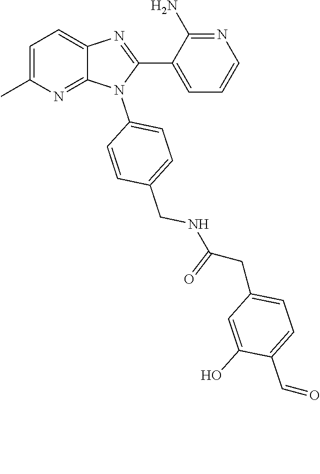 |
| 25 | 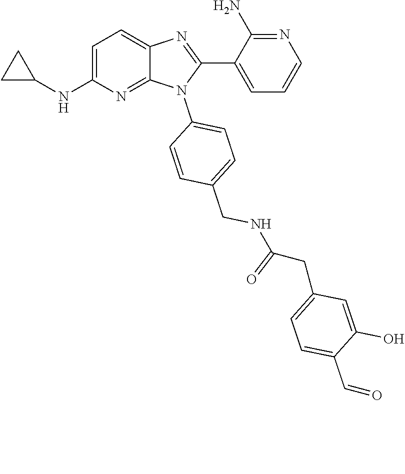 |
| 26 | 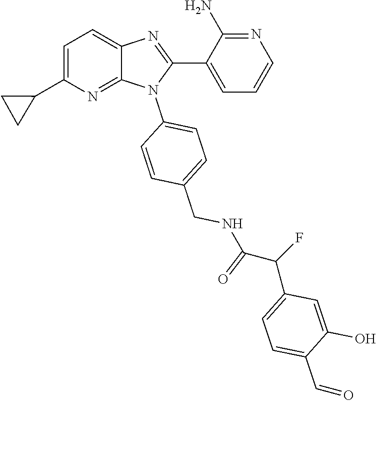 |
| 27 | 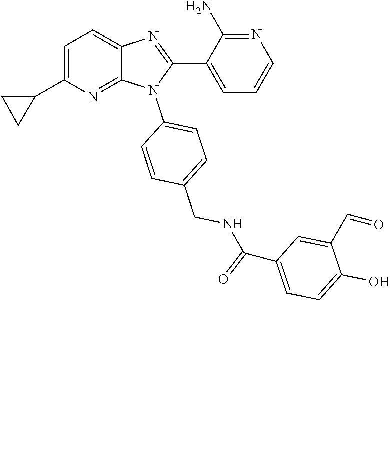 |
| 28 | 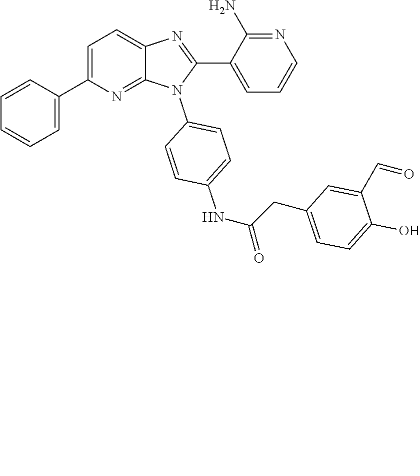 |
| 29 | 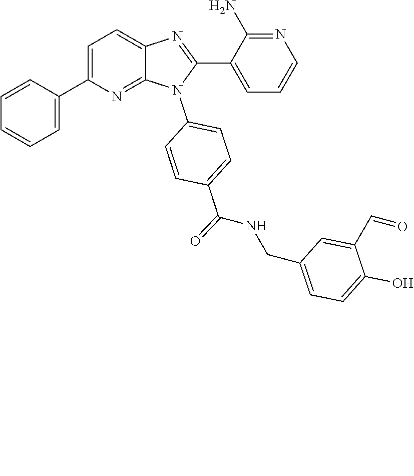 |
| 30 | 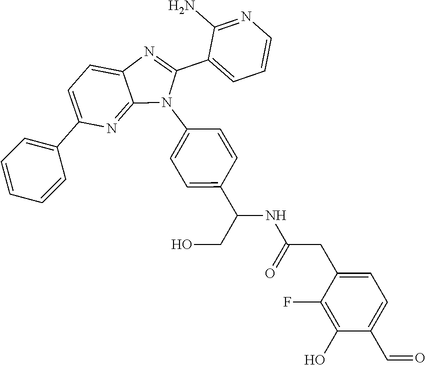 [SMILES: Nc1ncccc1-c1nc2ccc(-c3ccccc3)nc2n1-c1ccc(C(CO)NC(=O)Cc2ccc(C=O)c(O)c2F)cc1] |
| 31 | 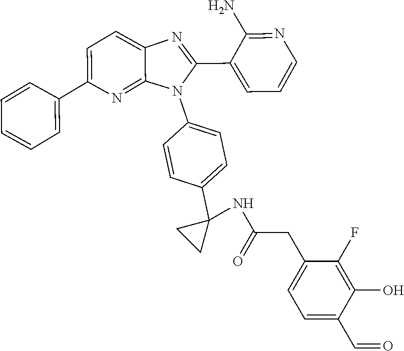 [SMILES: Nc1ncccc1-c1nc2ccc(-c3ccccc3)nc2n1-c1ccc(C2(NC(=O)Cc3ccc(C=O)c(O)c3F)CC2)cc1] |
| 32 | 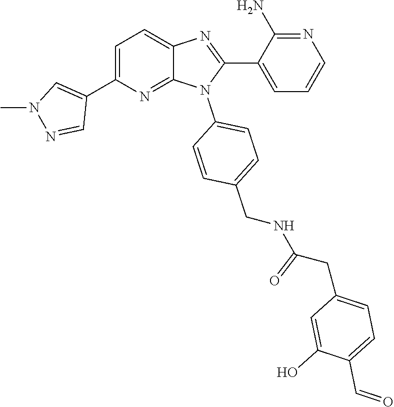 [SMILES: Cn1cc(-c2ccc3nc(-c4cccnc4N)n(-c4ccc(CNC(=O)Cc5ccc(C=O)c(O)c5)cc4)c3n2)cn1] |
| 33 | 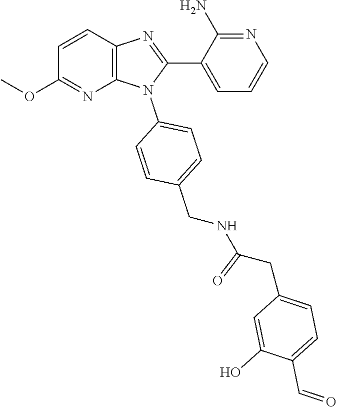 [SMILES: COc1ccc2nc(-c3cccnc3N)n(-c3ccc(CNC(=O)Cc4ccc(C=O)c(O)c4)cc3)c2n1] |
| 34 | 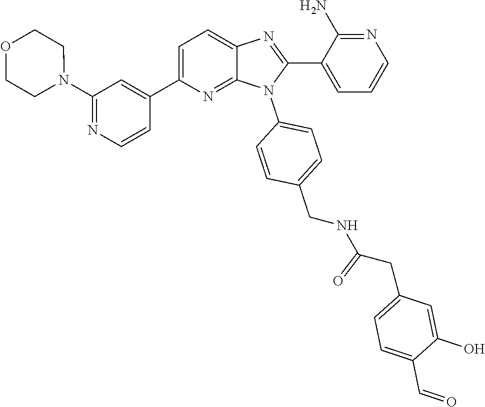 [SMILES: Nc1ncccc1-c1nc2ccc(-c3ccnc(N4CCOCC4)c3)nc2n1-c1ccc(CNC(=O)Cc2ccc(C=O)c(O)c2)cc1] |
| 35 | 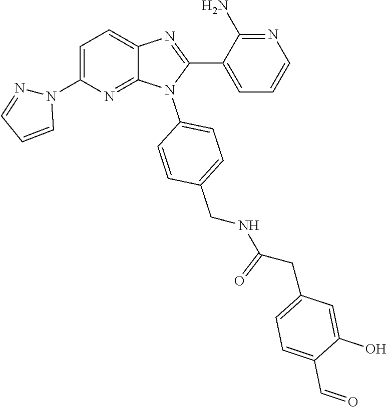 [SMILES: Nc1ncccc1-c1nc2ccc(-n3cccn3)nc2n1-c1ccc(CNC(=O)Cc2ccc(C=O)c(O)c2)cc1] |
| 36 | 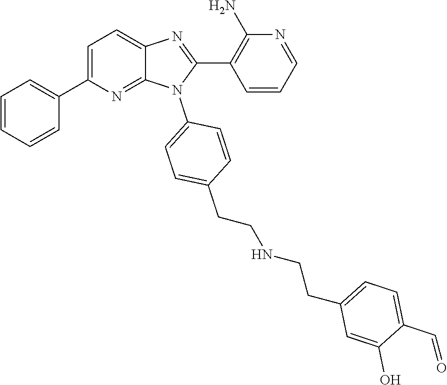 [SMILES: Nc1ncccc1-c1nc2ccc(-c3ccccc3)nc2n1-c1ccc(CCNCCc2ccc(C=O)c(O)c2)cc1] |
| 37 | 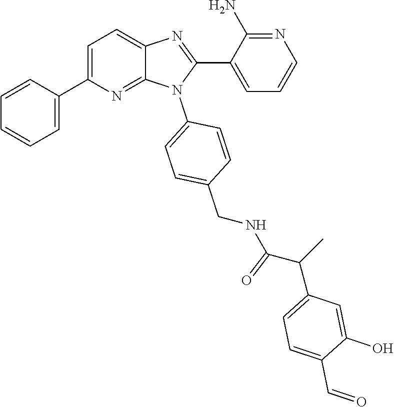 [SMILES: CC(C(=O)NCc1ccc(-n2c(-c3cccnc3N)nc3ccc(-c4ccccc4)nc32)cc1)c1ccc(C=O)c(O)c1] |
| 38 | 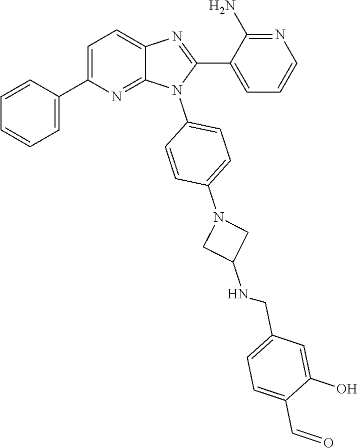 [SMILES: Nc1ncccc1-c1nc2ccc(-c3ccccc3)nc2n1-c1ccc(N2CC(NCc3ccc(C=O)c(O)c3)C2)cc1] |
| 39 | 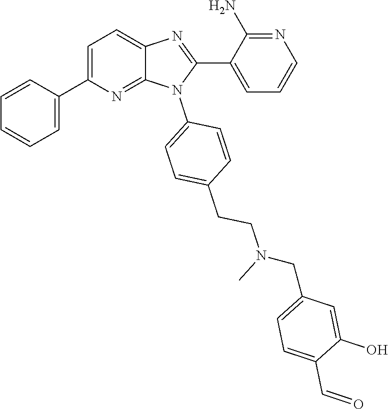 [SMILES: CN(CCc1ccc(-n2c(-c3cccnc3N)nc3ccc(-c4ccccc4)nc32)cc1)Cc1ccc(C=O)c(O)c1] |
| 40 | 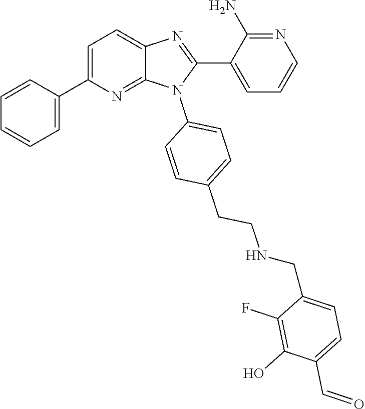 [SMILES: Nc1ncccc1-c1nc2ccc(-c3ccccc3)nc2n1-c1ccc(CCNCc2ccc(C=O)c(O)c2F)cc1] |
| 41 | 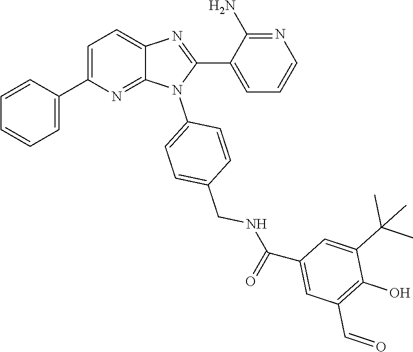 [SMILES: CC(C)(C)c1cc(C(=O)NCc2ccc(-n3c(-c4cccnc4N)nc4ccc(-c5ccccc5)nc43)cc2)cc(C=O)c1O] |
| 42 | 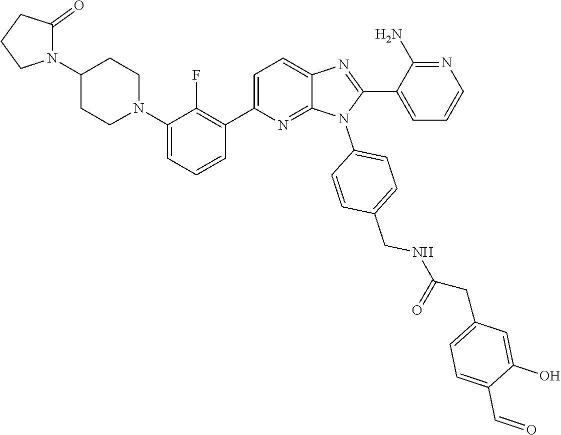 [SMILES: Nc1ncccc1-c1nc2ccc(-c3cccc(N4CCC(N5CCCC5=O)CC4)c3F)nc2n1-c1ccc(CNC(=O)Cc2ccc(C=O)c(O)c2)cc1] |
| 43 | 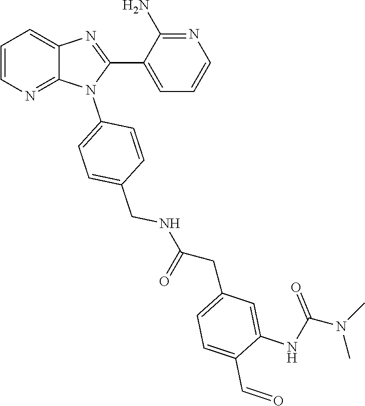 [SMILES: CN(C)C(=O)Nc1cc(CC(=O)NCc2ccc(-n3c(-c4cccnc4N)nc4cccnc43)cc2)ccc1C=O] |
| 44 | 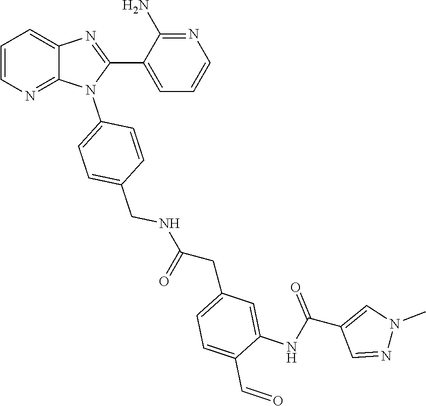 [SMILES: Cn1cc(C(=O)Nc2cc(CC(=O)NCc3ccc(-n4c(-c5cccnc5N)nc5cccnc54)cc3)ccc2C=O)cn1] |
| 45 | 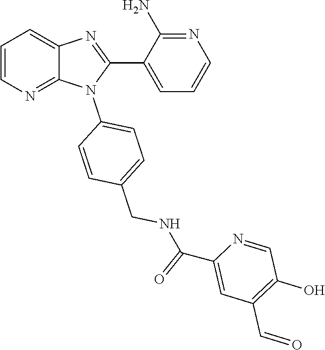 [SMILES: Nc1ncccc1-c1nc2cccnc2n1-c1ccc(CNC(=O)c2cc(C=O)c(O)cn2)cc1] |
| 46 | 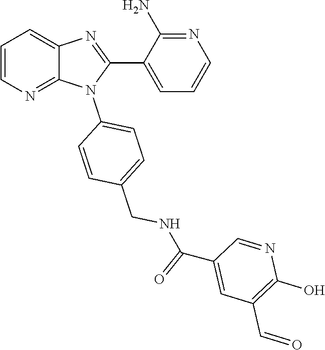 [SMILES: Nc1ncccc1-c1nc2cccnc2n1-c1ccc(CNC(=O)c2cnc(O)c(C=O)c2)cc1] |
| 47 | 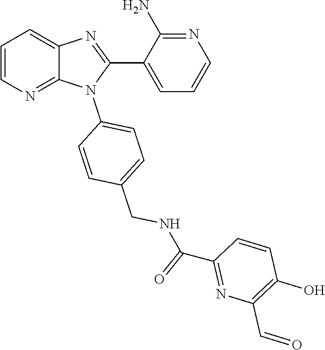 [SMILES: Nc1ncccc1-c1nc2cccnc2n1-c1ccc(CNC(=O)c2ccc(O)c(C=O)n2)cc1] |
| 48 | 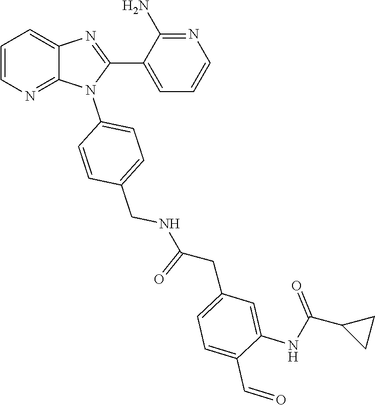 [SMILES: Nc1ncccc1-c1nc2cccnc2n1-c1ccc(CNC(=O)Cc2ccc(C=O)c(NC(=O)C3CC3)c2)cc1] |
| 49 | 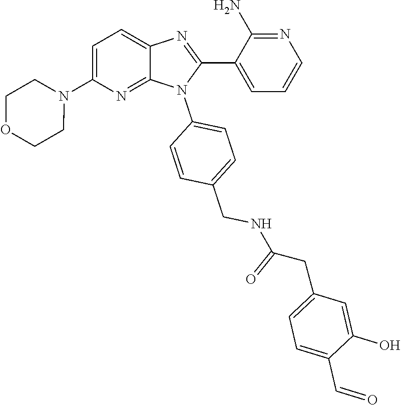 [SMILES: Nc1ncccc1-c1nc2ccc(N3CCOCC3)nc2n1-c1ccc(CNC(=O)Cc2ccc(C=O)c(O)c2)cc1] |
| 50 | 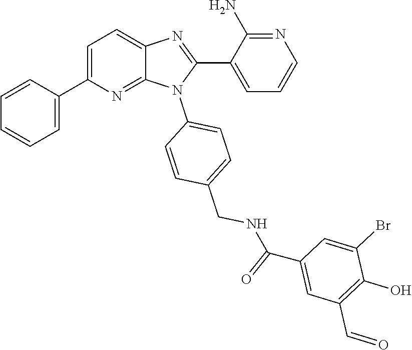 [SMILES: Nc1ncccc1-c1nc2ccc(-c3ccccc3)nc2n1-c1ccc(CNC(=O)c2cc(Br)c(O)c(C=O)c2)cc1] |
| 51 | 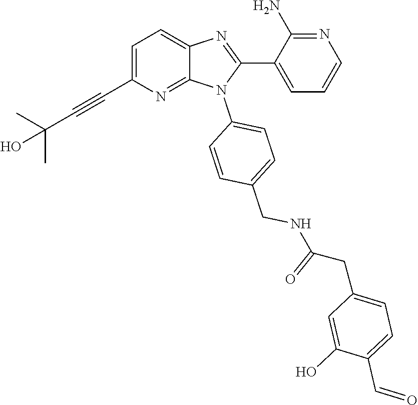 [SMILES: CC(C)(O)C#Cc1ccc2nc(-c3cccnc3N)n(-c3ccc(CNC(=O)Cc4ccc(C=O)c(O)c4)cc3)c2n1] |
| 52 | 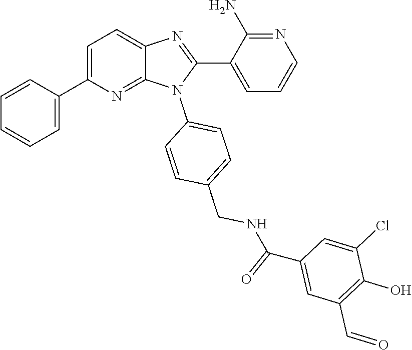 [SMILES: Nc1ncccc1-c1nc2ccc(-c3ccccc3)nc2n1-c1ccc(CNC(=O)c2cc(Cl)c(O)c(C=O)c2)cc1] |
| 53 | 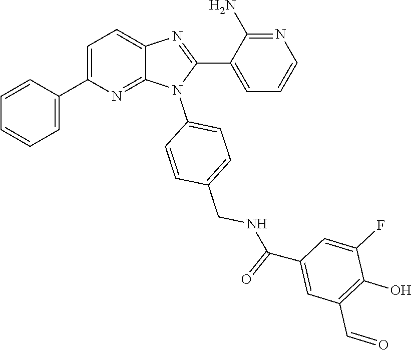 [SMILES: Nc1ncccc1-c1nc2ccc(-c3ccccc3)nc2n1-c1ccc(CNC(=O)c2cc(F)c(O)c(C=O)c2)cc1] |
| 54 | 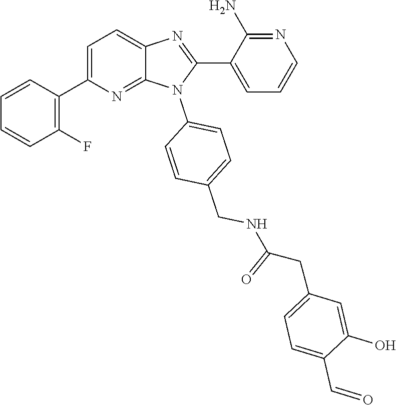 [SMILES: Nc1ncccc1-c1nc2ccc(-c3ccccc3F)nc2n1-c1ccc(CNC(=O)Cc2ccc(C=O)c(O)c2)cc1] |
| 55 | 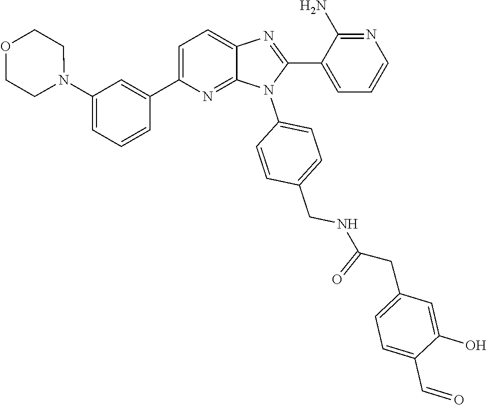 [SMILES: Nc1ncccc1-c1nc2ccc(-c3cccc(N4CCOCC4)c3)nc2n1-c1ccc(CNC(=O)Cc2ccc(C=O)c(O)c2)cc1] |
| 56 | 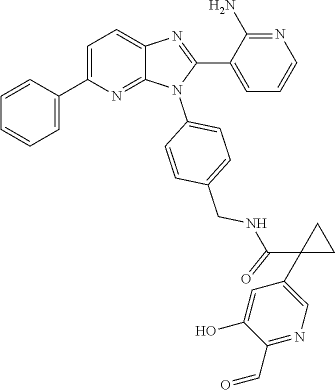 [SMILES: Nc1ncccc1-c1nc2ccc(-c3ccccc3)nc2n1-c1ccc(CNC(=O)C2(c3cnc(C=O)c(O)c3)CC2)cc1] |
| 57 | 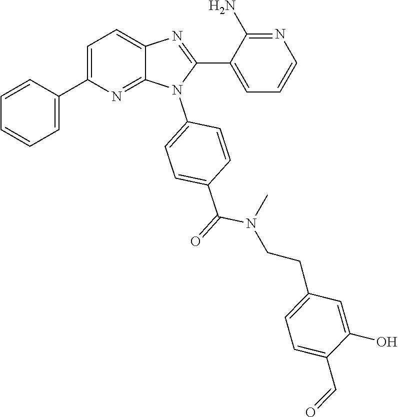 [SMILES: CN(CCc1ccc(C=O)c(O)c1)C(=O)c1ccc(-n2c(-c3cccnc3N)nc3ccc(-c4ccccc4)nc32)cc1] |
| 58 | 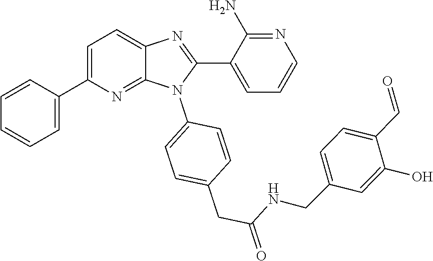 [SMILES: Nc1ncccc1-c1nc2ccc(-c3ccccc3)nc2n1-c1ccc(CC(=O)NCc2ccc(C=O)c(O)c2)cc1] |
| 59 | 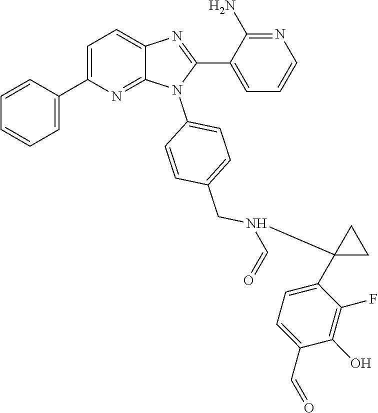 [SMILES: Nc1ncccc1-c1nc2ccc(-c3ccccc3)nc2n1-c1ccc(CN(C=O)C2(c3ccc(C=O)c(O)c3F)CC2)cc1] |
| 60 | 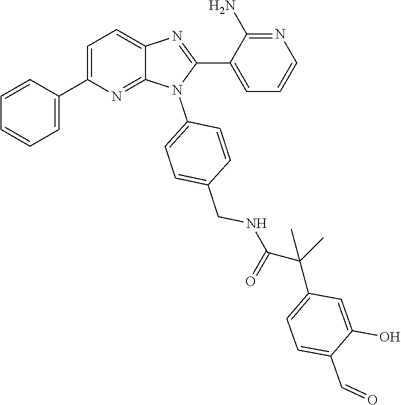 [SMILES: CC(C)(C(=O)NCc1ccc(-n2c(-c3cccnc3N)nc3ccc(-c4ccccc4)nc32)cc1)c1ccc(C=O)c(O)c1] |
| 61 | 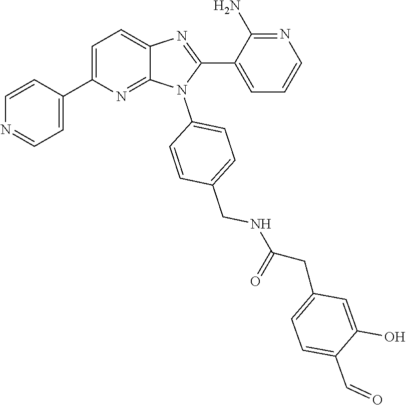 [SMILES: Nc1ncccc1-c1nc2ccc(-c3ccncc3)nc2n1-c1ccc(CNC(=O)Cc2ccc(C=O)c(O)c2)cc1] |
| 62 | 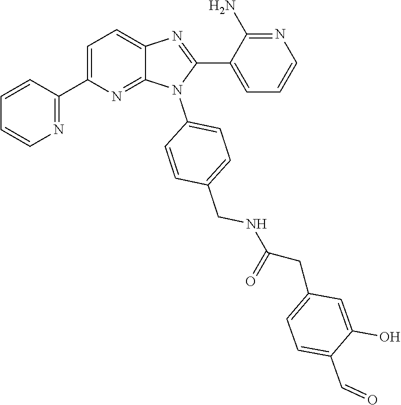 [SMILES: Nc1ncccc1-c1nc2ccc(-c3ccccn3)nc2n1-c1ccc(CNC(=O)Cc2ccc(C=O)c(O)c2)cc1] |
| 63 | 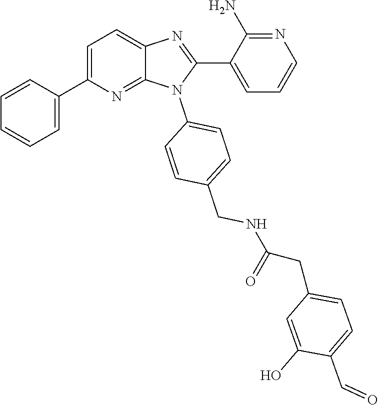 [SMILES: Nc1ncccc1-c1nc2ccc(-c3ccccc3)nc2n1-c1ccc(CNC(=O)Cc2ccc(C=O)c(O)c2)cc1] |
| 64 | 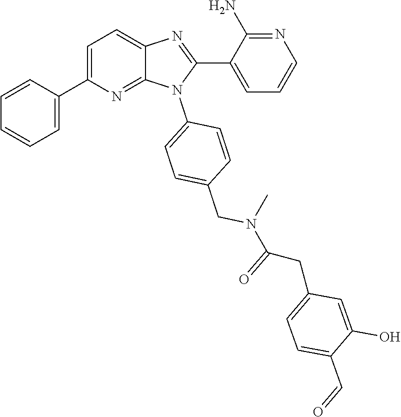 [SMILES: CN(Cc1ccc(-n2c(-c3cccnc3N)nc3ccc(-c4ccccc4)nc32)cc1)C(=O)Cc1ccc(C=O)c(O)c1] |
| 65 | 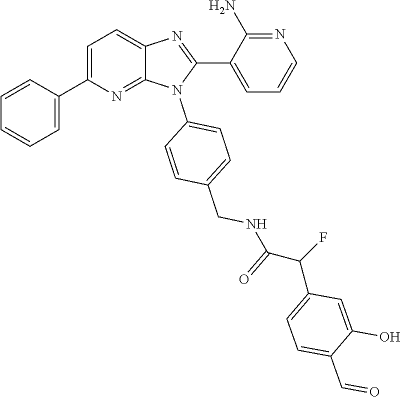 [SMILES: Nc1ncccc1-c1nc2ccc(-c3ccccc3)nc2n1-c1ccc(CNC(=O)C(F)c2ccc(C=O)c(O)c2)cc1] |
| 66 | 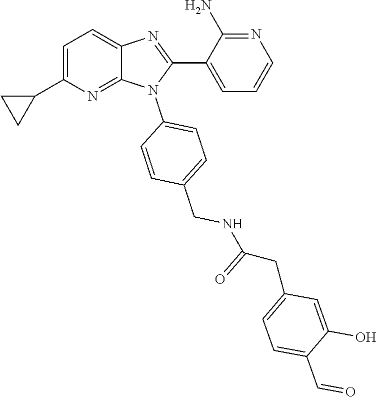 [SMILES: Nc1ncccc1-c1nc2ccc(C3CC3)nc2n1-c1ccc(CNC(=O)Cc2ccc(C=O)c(O)c2)cc1] |
| 67 | 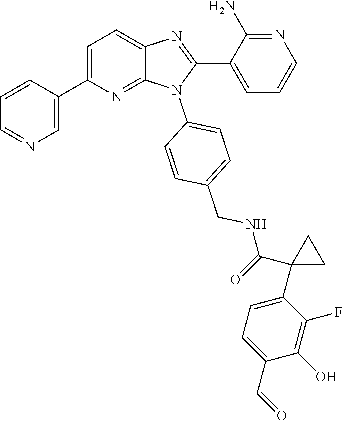 [SMILES: Nc1ncccc1-c1nc2ccc(-c3cccnc3)nc2n1-c1ccc(CNC(=O)C2(c3ccc(C=O)c(O)c3F)CC2)cc1] |
| 68 | 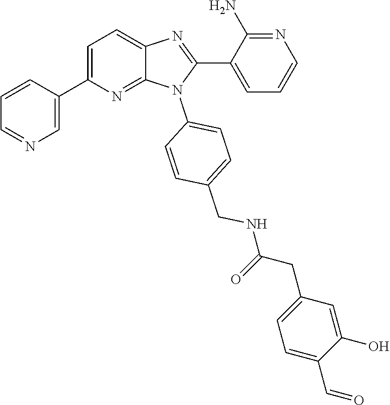 [SMILES: Nc1ncccc1-c1nc2ccc(-c3cccnc3)nc2n1-c1ccc(CNC(=O)Cc2ccc(C=O)c(O)c2)cc1] |
| 69 | 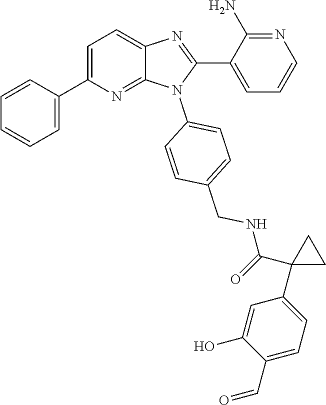 [SMILES: Nc1ncccc1-c1nc2ccc(-c3ccccc3)nc2n1-c1ccc(CNC(=O)C2(c3ccc(C=O)c(O)c3)CC2)cc1] |
| 70 | 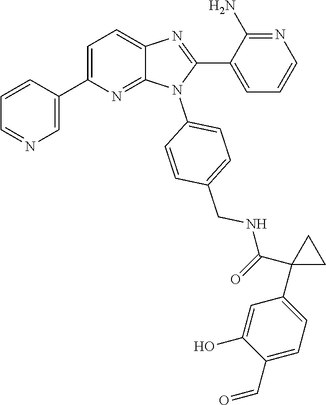 [SMILES: Nc1ncccc1-c1nc2ccc(-c3cccnc3)nc2n1-c1ccc(CNC(=O)C2(c3ccc(C=O)c(O)c3)CC2)cc1] |
| 71 | 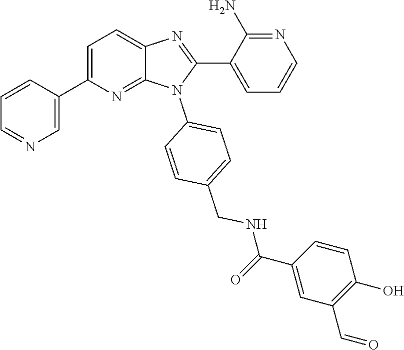 [SMILES: Nc1ncccc1-c1nc2ccc(-c3cccnc3)nc2n1-c1ccc(CNC(=O)c2ccc(O)c(C=O)c2)cc1] |
| 72 | 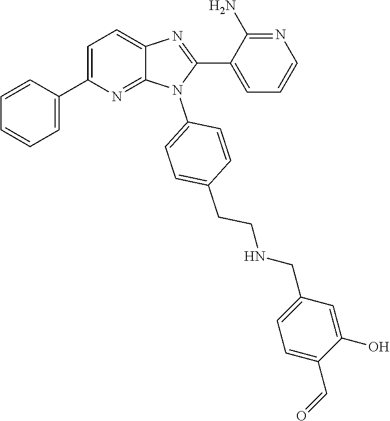 [SMILES: Nc1ncccc1-c1nc2ccc(-c3ccccc3)nc2n1-c1ccc(CCNCc2ccc(C=O)c(O)c2)cc1] |
| 73 | 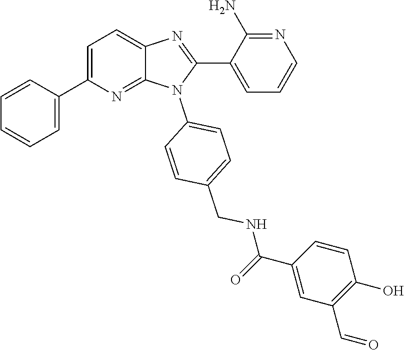 [SMILES: Nc1ncccc1-c1nc2ccc(-c3ccccc3)nc2n1-c1ccc(CNC(=O)c2ccc(O)c(C=O)c2)cc1] |
| 74 | 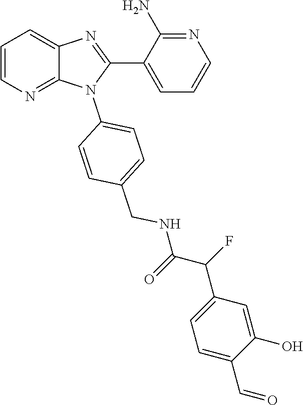 [SMILES: Nc1ncccc1-c1nc2cccnc2n1-c1ccc(CNC(=O)C(F)c2ccc(C=O)c(O)c2)cc1] |
| 75 | 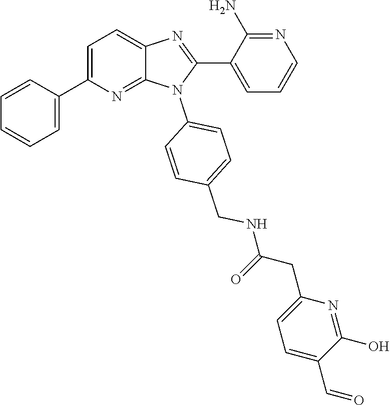 [SMILES: Nc1ncccc1-c1nc2ccc(-c3ccccc3)nc2n1-c1ccc(CNC(=O)Cc2ccc(C=O)c(O)n2)cc1] |
| 76 | 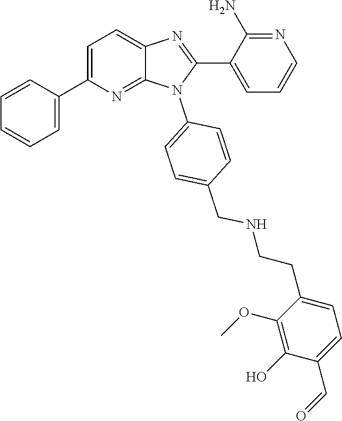 [SMILES: COc1c(CCNCc2ccc(-n3c(-c4cccnc4N)nc4ccc(-c5ccccc5)nc43)cc2)ccc(C=O)c1O] |
| 77 | 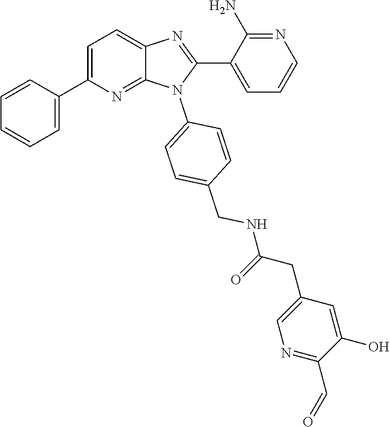 [SMILES: Nc1ncccc1-c1nc2ccc(-c3ccccc3)nc2n1-c1ccc(CNC(=O)Cc2cnc(C=O)c(O)c2)cc1] |
| 78 | 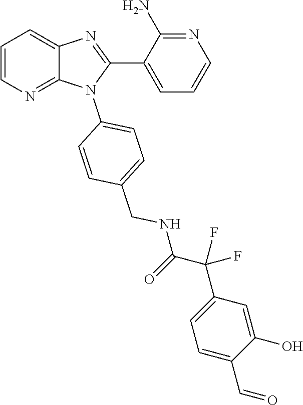 [SMILES: Nc1ncccc1-c1nc2cccnc2n1-c1ccc(CNC(=O)C(F)(F)c2ccc(C=O)c(O)c2)cc1] |
| 79 | 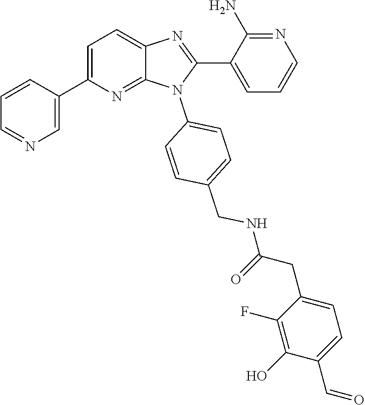 [SMILES: Nc1ncccc1-c1nc2ccc(-c3cccnc3)nc2n1-c1ccc(CNC(=O)Cc2ccc(C=O)c(O)c2F)cc1] |
| 80 | 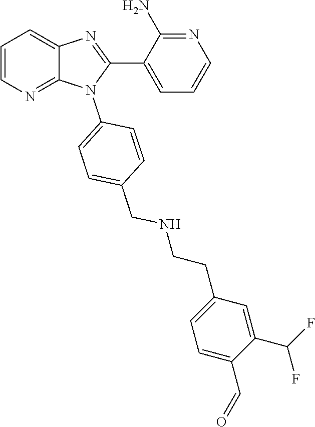 [SMILES: Nc1ncccc1-c1nc2cccnc2n1-c1ccc(CNCCc2ccc(C=O)c(C(F)F)c2)cc1] |
| 81 | 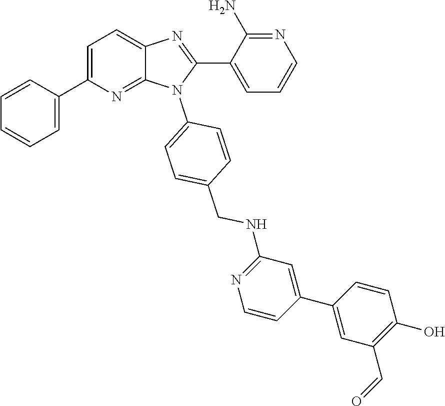 [SMILES: Nc1ncccc1-c1nc2ccc(-c3ccccc3)nc2n1-c1ccc(CNc2cc(-c3ccc(O)c(C=O)c3)ccn2)cc1] |
| 82 | 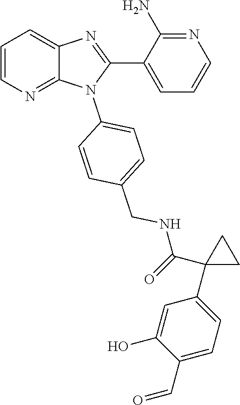 [SMILES: Nc1ncccc1-c1nc2cccnc2n1-c1ccc(CNC(=O)C2(c3ccc(C=O)c(O)c3)CC2)cc1] |
| 83 | 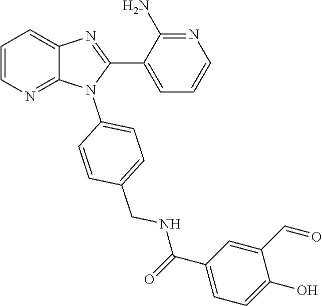 [SMILES: Nc1ncccc1-c1nc2cccnc2n1-c1ccc(CNC(=O)c2ccc(O)c(C=O)c2)cc1] |
| 84 | 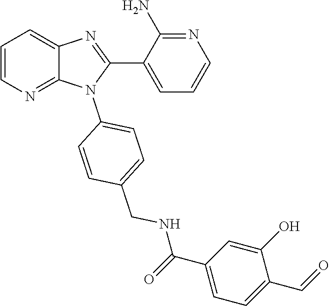 [SMILES: Nc1ncccc1-c1nc2cccnc2n1-c1ccc(CNC(=O)c2ccc(C=O)c(O)c2)cc1] |
| 85 | 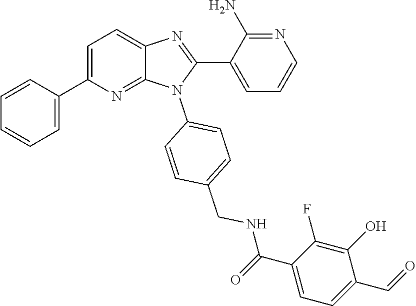 [SMILES: Nc1ncccc1-c1nc2ccc(-c3ccccc3)nc2n1-c1ccc(CNC(=O)c2ccc(C=O)c(O)c2F)cc1] |
| 86 | 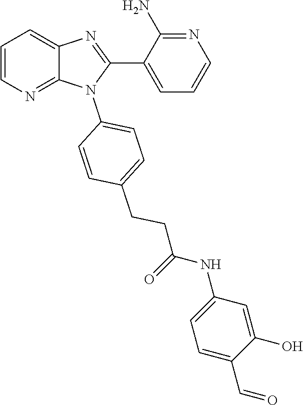 [SMILES: Nc1ncccc1-c1nc2cccnc2n1-c1ccc(CCC(=O)Nc2ccc(C=O)c(O)c2)cc1] |
| 87 | 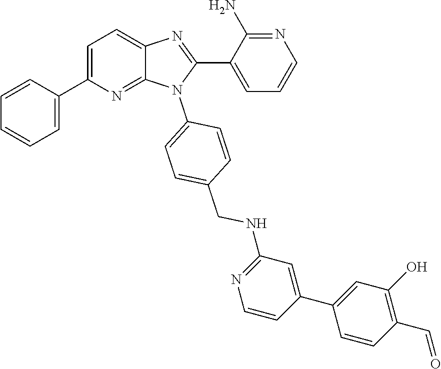 [SMILES: Nc1ncccc1-c1nc2ccc(-c3ccccc3)nc2n1-c1ccc(CNc2cc(-c3ccc(C=O)c(O)c3)ccn2)cc1] |
| 88 | 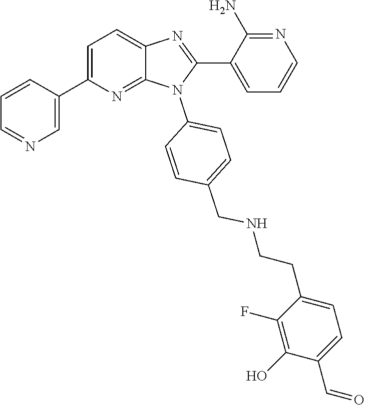 [SMILES: Nc1ncccc1-c1nc2ccc(-c3cccnc3)nc2n1-c1ccc(CNCCc2ccc(C=O)c(O)c2F)cc1] |
| 89 | 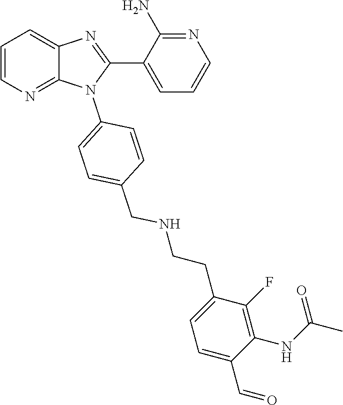 [SMILES: CC(=O)Nc1c(C=O)ccc(CCNCc2ccc(-n3c(-c4cccnc4N)nc4cccnc43)cc2)c1F] |
| 90 | 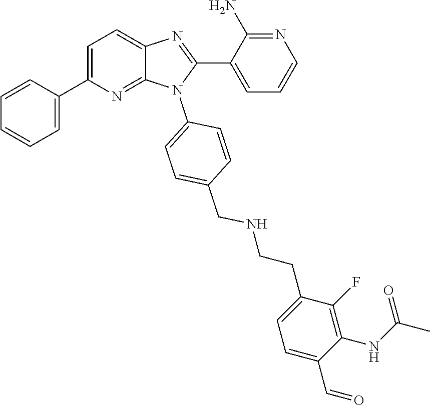 [SMILES: CC(=O)Nc1c(C=O)ccc(CCNCc2ccc(-n3c(-c4cccnc4N)nc4ccc(-c5ccccc5)nc43)cc2)c1F] |
| 91 | 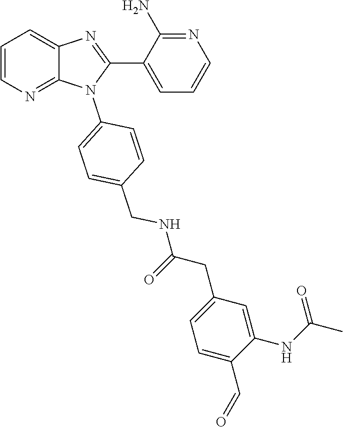 [SMILES: CC(=O)Nc1cc(CC(=O)NCc2ccc(-n3c(-c4cccnc4N)nc4cccnc43)cc2)ccc1C=O] |
| 92 | 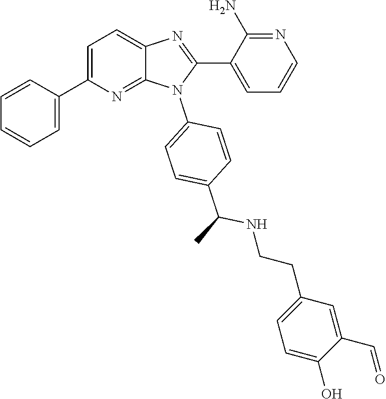 [SMILES: C[C@H](NCCc1ccc(O)c(C=O)c1)c1ccc(-n2c(-c3cccnc3N)nc3ccc(-c4ccccc4)nc32)cc1] |
| 93 | 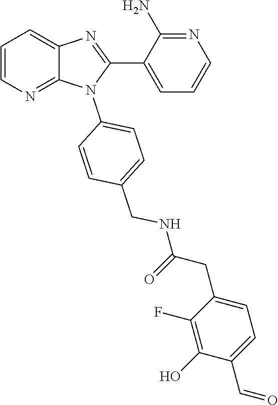 [SMILES: Nc1ncccc1-c1nc2cccnc2n1-c1ccc(CNC(=O)Cc2ccc(C=O)c(O)c2F)cc1] |
| 94 | 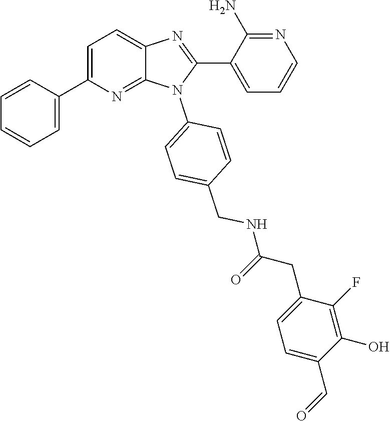 [SMILES: Nc1ncccc1-c1nc2ccc(-c3ccccc3)nc2n1-c1ccc(CNC(=O)Cc2ccc(C=O)c(O)c2F)cc1] |
| 95 | 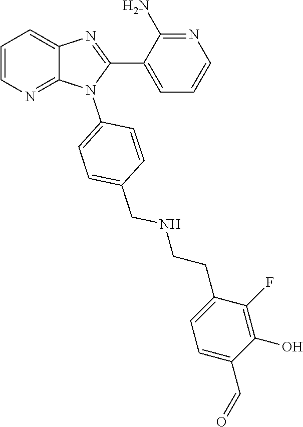 [SMILES: Nc1ncccc1-c1nc2cccnc2n1-c1ccc(CNCCc2ccc(C=O)c(O)c2F)cc1] |
| 96 | 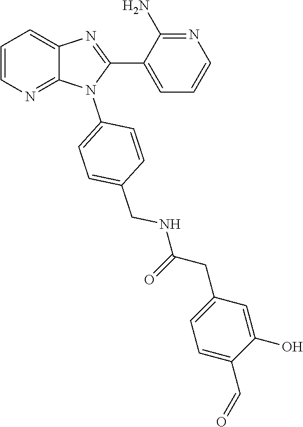 [SMILES: Nc1ncccc1-c1nc2cccnc2n1-c1ccc(CNC(=O)Cc2ccc(C=O)c(O)c2)cc1] |
| 97 | 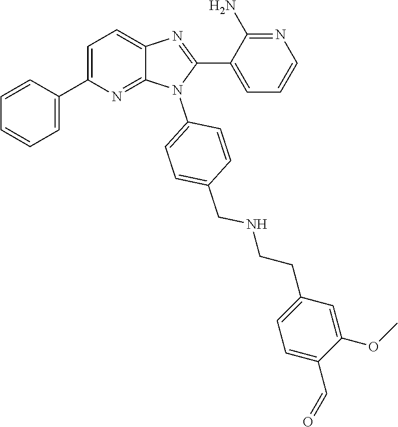 [SMILES: COc1cc(CCNCc2ccc(-n3c(-c4cccnc4N)nc4ccc(-c5ccccc5)nc43)cc2)ccc1C=O] |
| 98 | 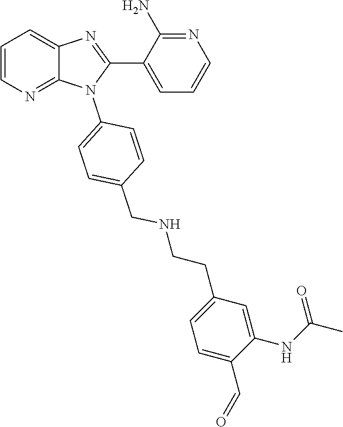 [SMILES: CC(=O)Nc1cc(CCNCc2ccc(-n3c(-c4cccnc4N)nc4cccnc43)cc2)ccc1C=O] |
| 99 | 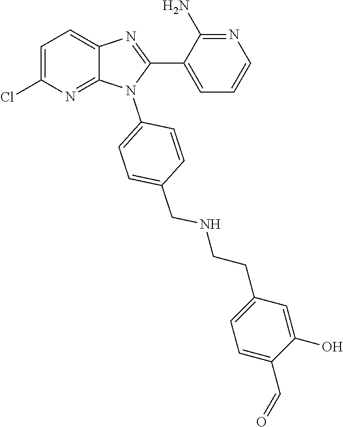 [SMILES: Nc1ncccc1-c1nc2ccc(Cl)nc2n1-c1ccc(CNCCc2ccc(C=O)c(O)c2)cc1] |
| 100 | 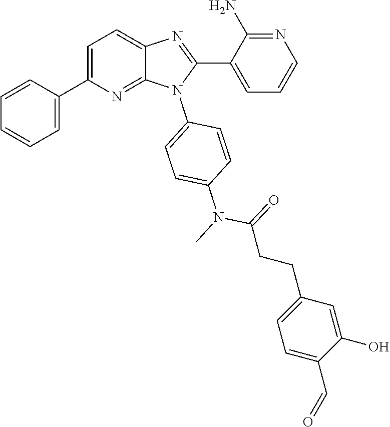 [SMILES: CN(C(=O)CCc1ccc(C=O)c(O)c1)c1ccc(-n2c(-c3cccnc3N)nc3ccc(-c4ccccc4)nc32)cc1] |
| 101 | 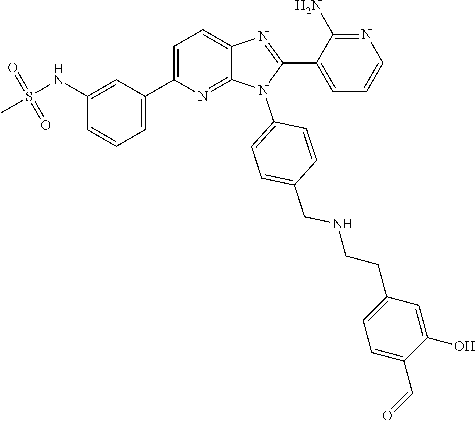 [SMILES: CS(=O)(=O)Nc1cccc(-c2ccc3nc(-c4cccnc4N)n(-c4ccc(CNCCc5ccc(C=O)c(O)c5)cc4)c3n2)c1] |
| 102 | 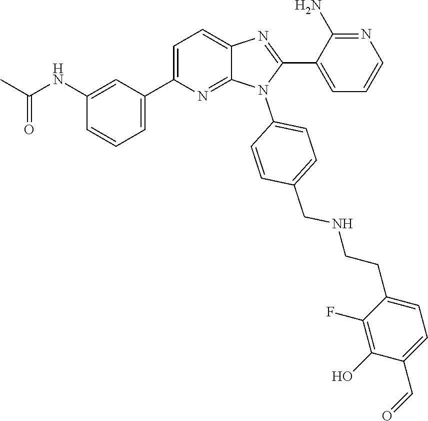 [SMILES: CC(=O)Nc1cccc(-c2ccc3nc(-c4cccnc4N)n(-c4ccc(CNCCc5ccc(C=O)c(O)c5F)cc4)c3n2)c1] |
| 103 | 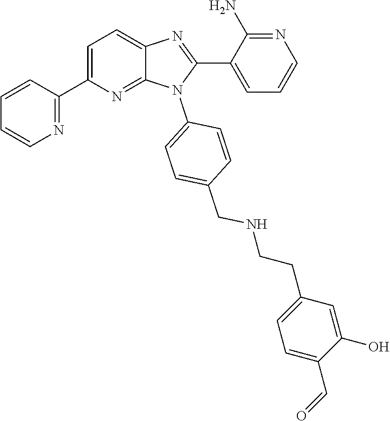 [SMILES: Nc1ncccc1-c1nc2ccc(-c3ccccn3)nc2n1-c1ccc(CNCCc2ccc(C=O)c(O)c2)cc1] |
| 104 | 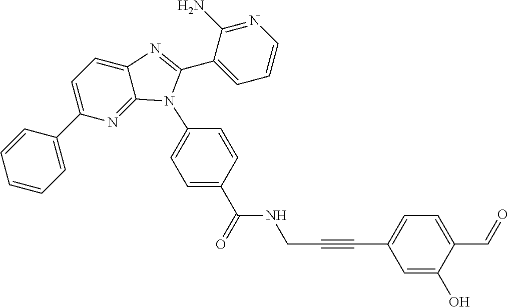 [SMILES: Nc1ncccc1-c1nc2ccc(-c3ccccc3)nc2n1-c1ccc(C(=O)NCC#Cc2ccc(C=O)c(O)c2)cc1] |
| 105 | 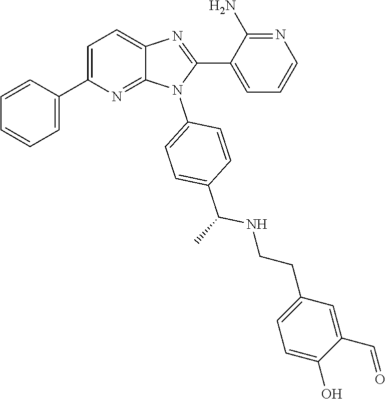 [SMILES: C[C@@H](NCCc1ccc(O)c(C=O)c1)c1ccc(-n2c(-c3cccnc3N)nc3ccc(-c4ccccc4)nc32)cc1] |
| 106 | 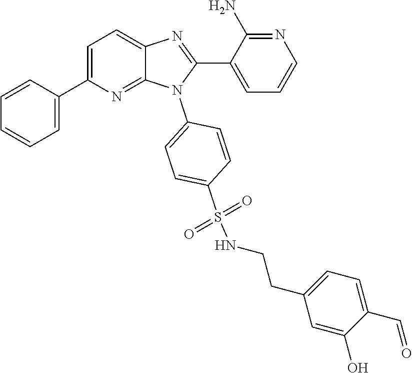 [SMILES: Nc1ncccc1-c1nc2ccc(-c3ccccc3)nc2n1-c1ccc(S(=O)(=O)NCCc2ccc(C=O)c(O)c2)cc1] |
| 107 | 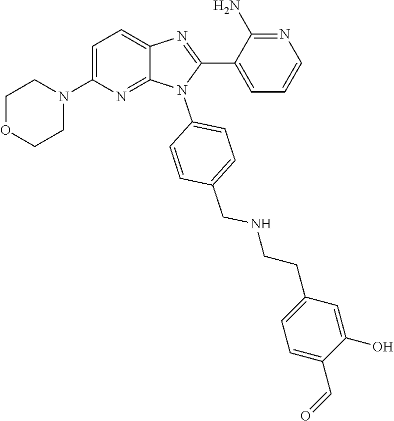 [SMILES: Nc1ncccc1-c1nc2ccc(N3CCOCC3)nc2n1-c1ccc(CNCCc2ccc(C=O)c(O)c2)cc1] |
| 108 | 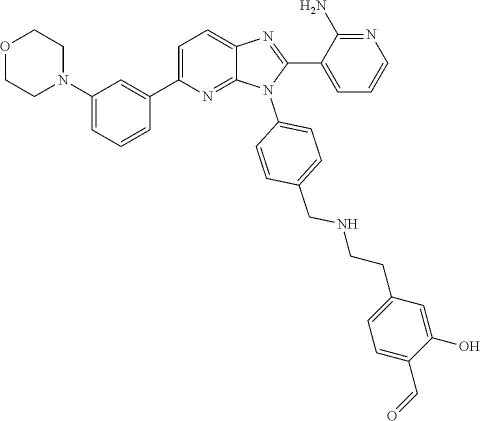 [SMILES: Nc1ncccc1-c1nc2ccc(-c3cccc(N4CCOCC4)c3)nc2n1-c1ccc(CNCCc2ccc(C=O)c(O)c2)cc1] |
| 109 | 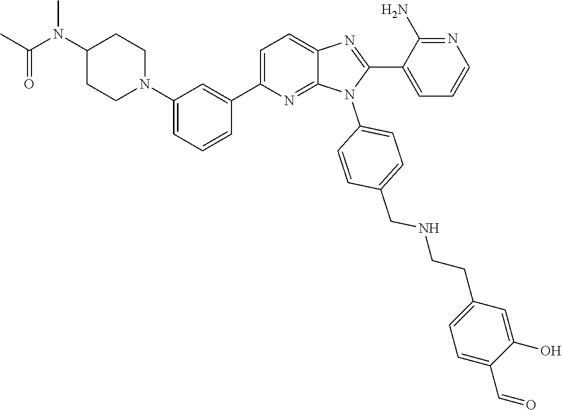 [SMILES: CC(=O)N(C)C1CCN(c2cccc(-c3ccc4nc(-c5cccnc5N)n(-c5ccc(CNCCc6ccc(C=O)c(O)c6)cc5)c4n3)c2)CC1] |
| 110 | 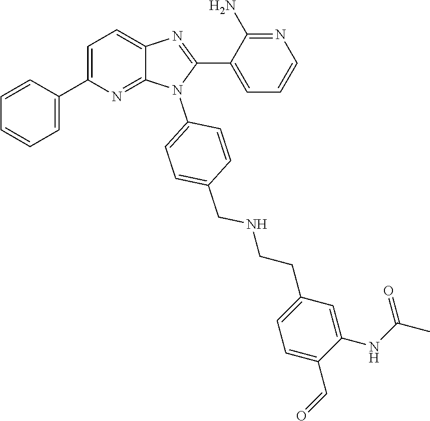 [SMILES: CC(=O)Nc1cc(CCNCc2ccc(-n3c(-c4cccnc4N)nc4ccc(-c5ccccc5)nc43)cc2)ccc1C=O] |
| 111 | 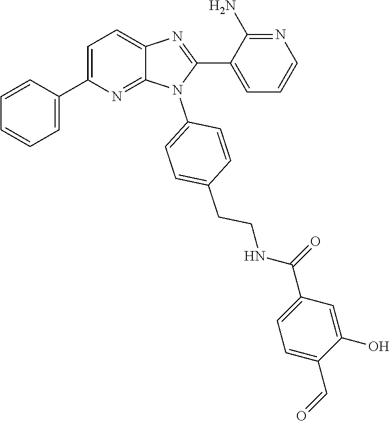 [SMILES: Nc1ncccc1-c1nc2ccc(-c3ccccc3)nc2n1-c1ccc(CCNC(=O)c2ccc(C=O)c(O)c2)cc1] |
| 112 | 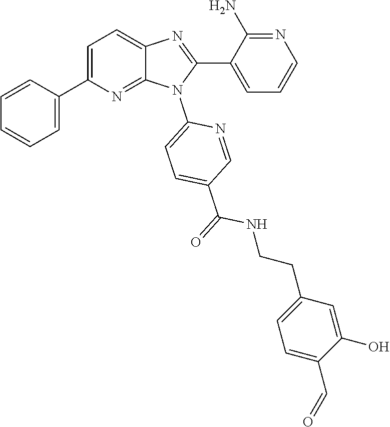 [SMILES: Nc1ncccc1-c1nc2ccc(-c3ccccc3)nc2n1-c1ccc(C(=O)NCCc2ccc(C=O)c(O)c2)cn1] |
| 113 | 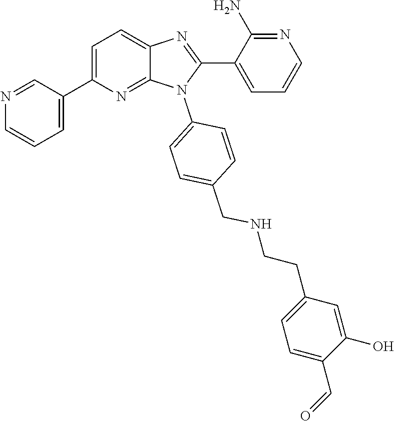 [SMILES: Nc1ncccc1-c1nc2ccc(-c3cccnc3)nc2n1-c1ccc(CNCCc2ccc(C=O)c(O)c2)cc1] |
| 114 | 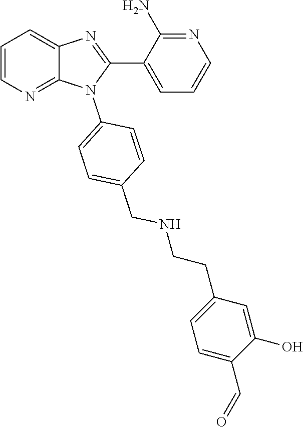 [SMILES: Nc1ncccc1-c1nc2cccnc2n1-c1ccc(CNCCc2ccc(C=O)c(O)c2)cc1] |
| 115 | 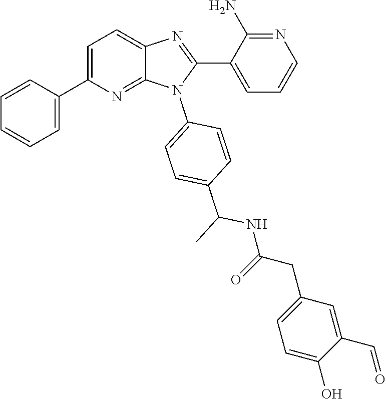 [SMILES: CC(NC(=O)Cc1ccc(O)c(C=O)c1)c1ccc(-n2c(-c3cccnc3N)nc3ccc(-c4ccccc4)nc32)cc1] |
| 116 | 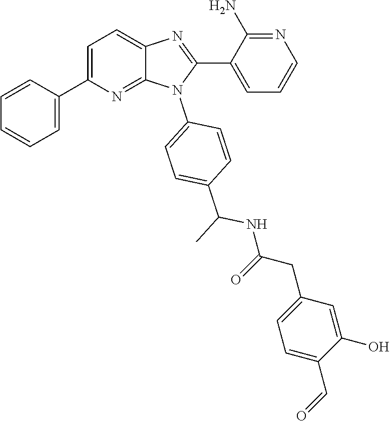 [SMILES: CC(NC(=O)Cc1ccc(C=O)c(O)c1)c1ccc(-n2c(-c3cccnc3N)nc3ccc(-c4ccccc4)nc32)cc1] |
| 117 | 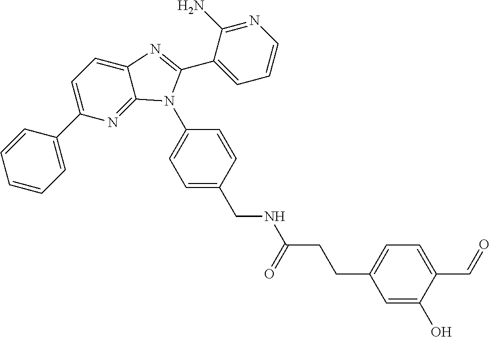 [SMILES: Nc1ncccc1-c1nc2ccc(-c3ccccc3)nc2n1-c1ccc(CNC(=O)CCc2ccc(C=O)c(O)c2)cc1] |
| 118 | 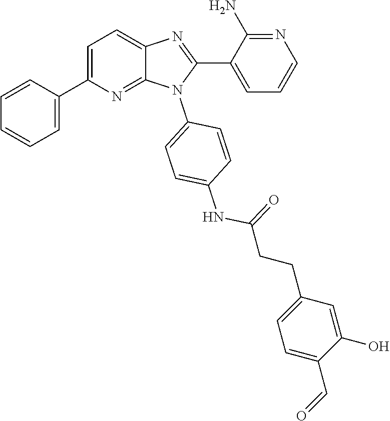 [SMILES: Nc1ncccc1-c1nc2ccc(-c3ccccc3)nc2n1-c1ccc(NC(=O)CCc2ccc(C=O)c(O)c2)cc1] |
| 119 | 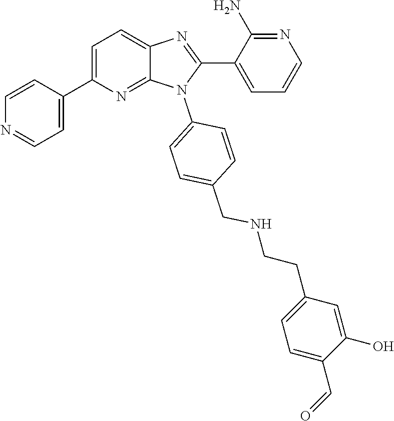 [SMILES: Nc1ncccc1-c1nc2ccc(-c3ccncc3)nc2n1-c1ccc(CNCCc2ccc(C=O)c(O)c2)cc1] |
| 120 | 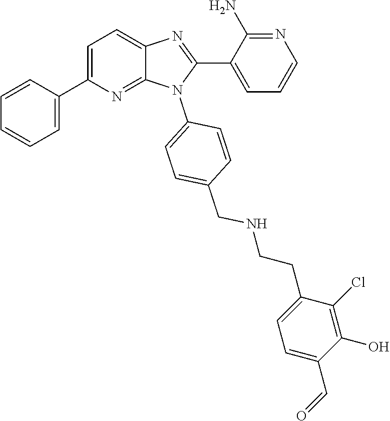 [SMILES: Nc1ncccc1-c1nc2ccc(-c3ccccc3)nc2n1-c1ccc(CNCCc2ccc(C=O)c(O)c2Cl)cc1] |
| 121 | 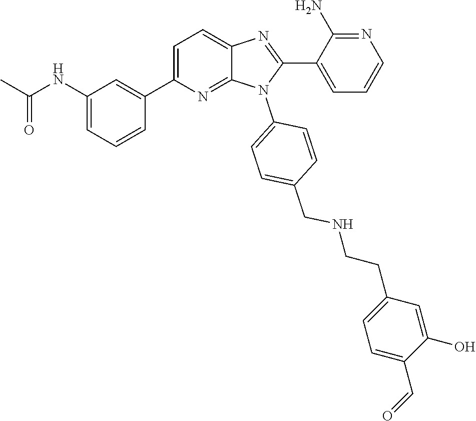 [SMILES: CC(=O)Nc1cccc(-c2ccc3nc(-c4cccnc4N)n(-c4ccc(CNCCc5ccc(C=O)c(O)c5)cc4)c3n2)c1] |
| 122 | 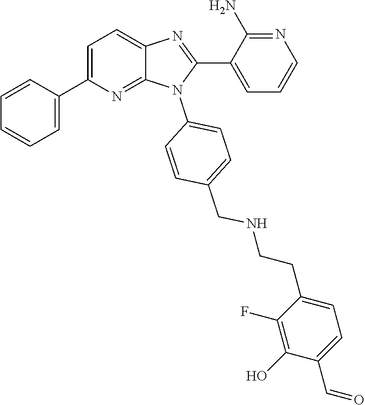 [SMILES: Nc1ncccc1-c1nc2ccc(-c3ccccc3)nc2n1-c1ccc(CNCCc2ccc(C=O)c(O)c2F)cc1] |
| 123 | 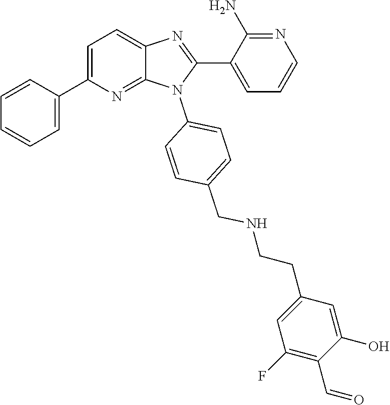 [SMILES: Nc1ncccc1-c1nc2ccc(-c3ccccc3)nc2n1-c1ccc(CNCCc2cc(O)c(C=O)c(F)c2)cc1] |
| 124 | 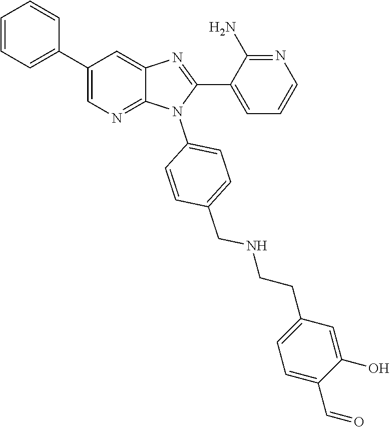 [SMILES: Nc1ncccc1-c1nc2cc(-c3ccccc3)cnc2n1-c1ccc(CNCCc2ccc(C=O)c(O)c2)cc1] |
| 125 | 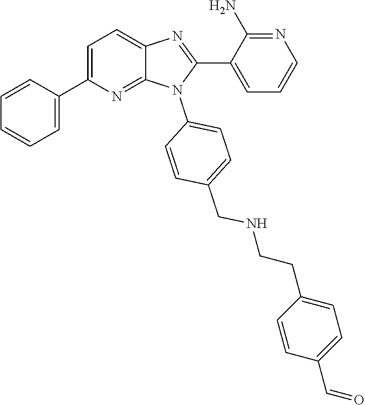 [SMILES: Nc1ncccc1-c1nc2ccc(-c3ccccc3)nc2n1-c1ccc(CNCCc2ccc(C=O)cc2)cc1] |
| 126 | 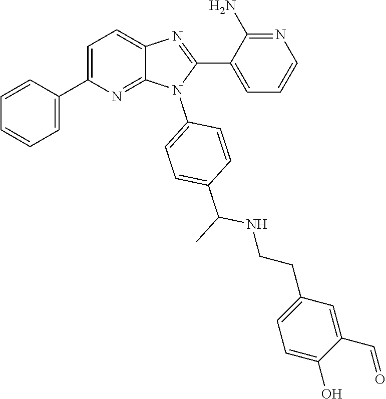 [SMILES: CC(NCCc1ccc(O)c(C=O)c1)c1ccc(-n2c(-c3cccnc3N)nc3ccc(-c4ccccc4)nc32)cc1] |
| 127 | 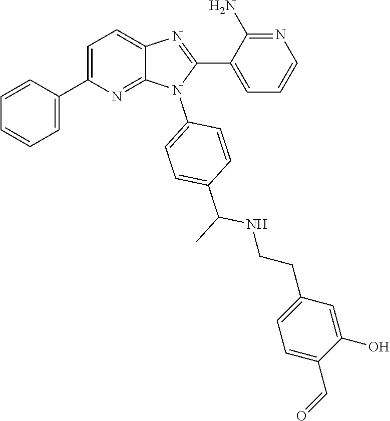 [SMILES: CC(NCCc1ccc(C=O)c(O)c1)c1ccc(-n2c(-c3cccnc3N)nc3ccc(-c4ccccc4)nc32)cc1] |
| 128 | 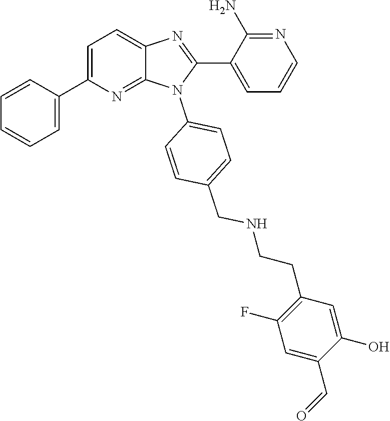 [SMILES: Nc1ncccc1-c1nc2ccc(-c3ccccc3)nc2n1-c1ccc(CNCCc2cc(O)c(C=O)cc2F)cc1] |
| 129 | 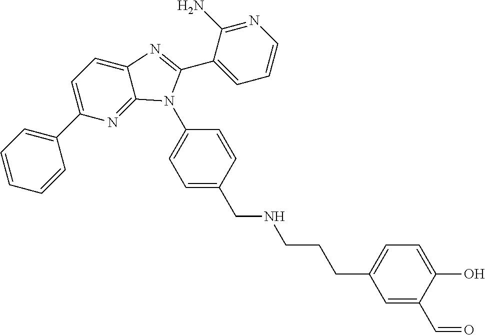 [SMILES: Nc1ncccc1-c1nc2ccc(-c3ccccc3)nc2n1-c1ccc(CNCCCc2ccc(O)c(C=O)c2)cc1] |
| 130 | 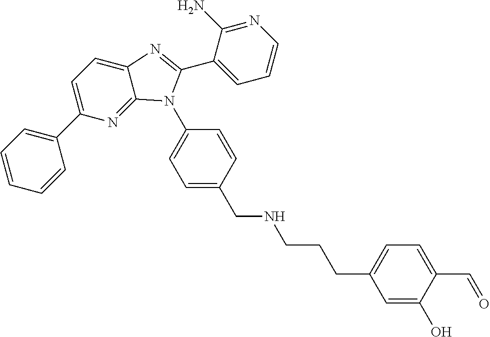 [SMILES: Nc1ncccc1-c1nc2ccc(-c3ccccc3)nc2n1-c1ccc(CNCCCc2ccc(C=O)c(O)c2)cc1] |
| 131 | 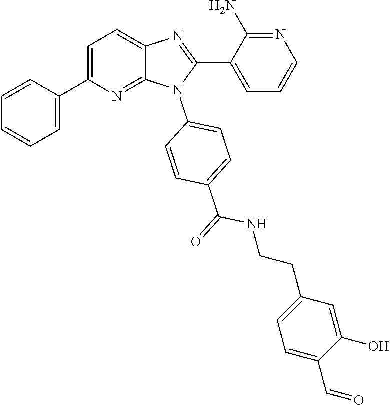 [SMILES: Nc1ncccc1-c1nc2ccc(-c3ccccc3)nc2n1-c1ccc(C(=O)NCCc2ccc(C=O)c(O)c2)cc1] |
| 132 | 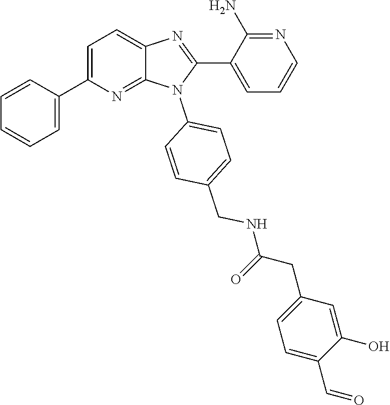 [SMILES: Nc1ncccc1-c1nc2ccc(-c3ccccc3)nc2n1-c1ccc(CNC(=O)Cc2ccc(C=O)c(O)c2)cc1] |
| 133 | 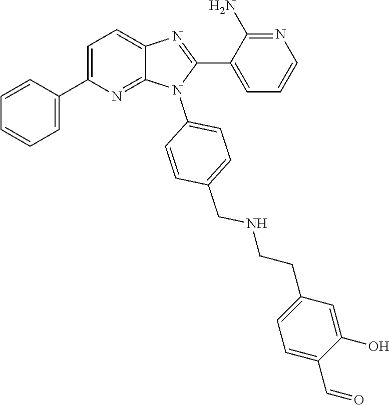 [SMILES: Nc1ncccc1-c1nc2ccc(-c3ccccc3)nc2n1-c1ccc(CNCCc2ccc(C=O)c(O)c2)cc1] |
| 134 | 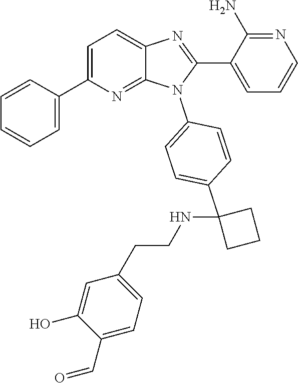 [SMILES: Nc1ncccc1-c1nc2ccc(-c3ccccc3)nc2n1-c1ccc(C2(NCCc3ccc(C=O)c(O)c3)CCC2)cc1] |
| 135 | 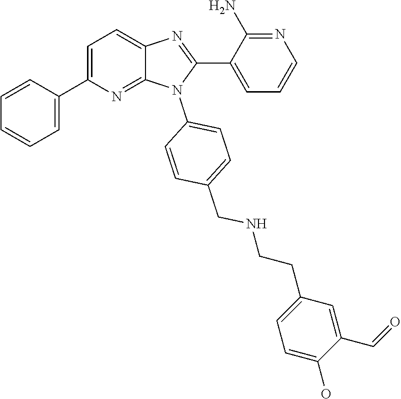 [SMILES: Nc1ncccc1-c1nc2ccc(-c3ccccc3)nc2n1-c1ccc(CNCCc2ccc(O)c(C=O)c2)cc1] |
| 136 | 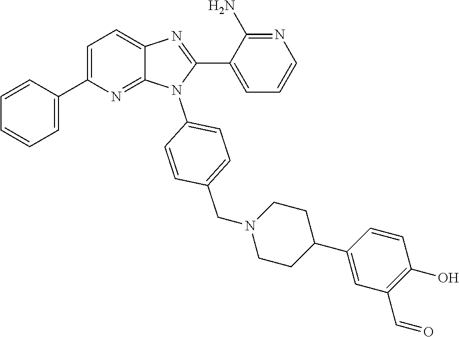 [SMILES: Nc1ncccc1-c1nc2ccc(-c3ccccc3)nc2n1-c1ccc(CN2CCC(c3ccc(O)c(C=O)c3)CC2)cc1] |
| 137 | 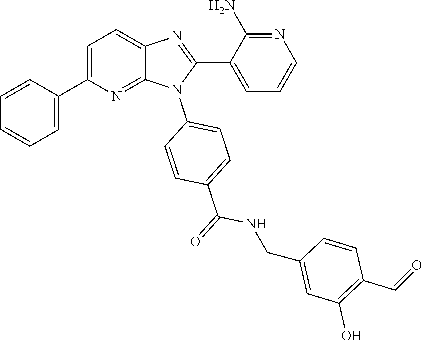 [SMILES: Nc1ncccc1-c1nc2ccc(-c3ccccc3)nc2n1-c1ccc(C(=O)NCc2ccc(C=O)c(O)c2)cc1] |
| 138 | 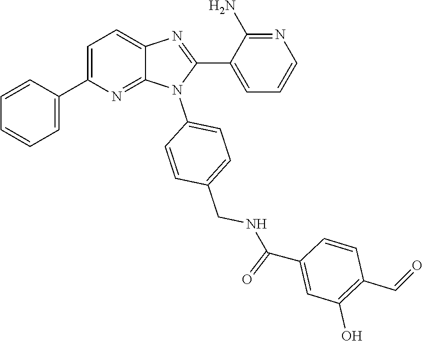 [SMILES: Nc1ncccc1-c1nc2ccc(-c3ccccc3)nc2n1-c1ccc(CNC(=O)c2ccc(C=O)c(O)c2)cc1] |
| 139 | 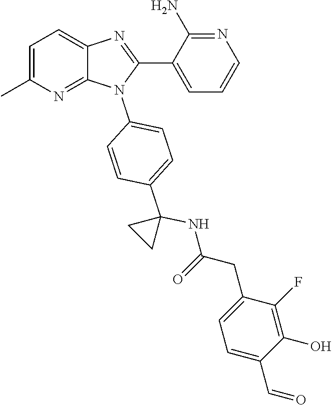 [SMILES: Cc1ccc2nc(-c3cccnc3N)n(-c3ccc(C4(NC(=O)Cc5ccc(C=O)c(O)c5F)CC4)cc3)c2n1] |
| 140 | 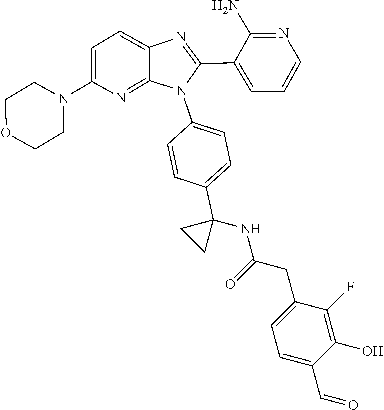 [SMILES: Nc1ncccc1-c1nc2ccc(N3CCOCC3)nc2n1-c1ccc(C2(NC(=O)Cc3ccc(C=O)c(O)c3F)CC2)cc1] |
| 141 | 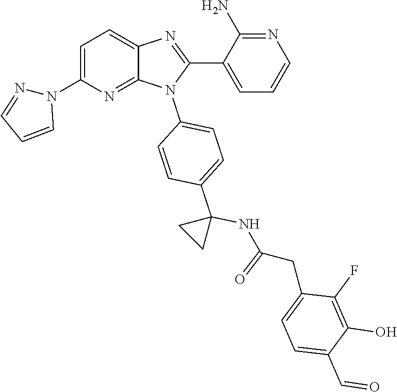 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z- or E- form (or cis- or trans-form). Furthermore, some chemical entities may exist in various tautomeric forms. Unless otherwise specified, compounds or salts of Formula (I), (I-A), (II), (II-A), (II-B), or (II-C), are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formula (I), (I-A), (II), (II-A), (II-B), or (II-C) herein may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formula (I), (I-A), (II), (II-A), (II-B), or (II-C), may comprise two or more enantiomers or diatereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formula (I), (I-A), (II), (II-A), (II-B), or (II-C), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

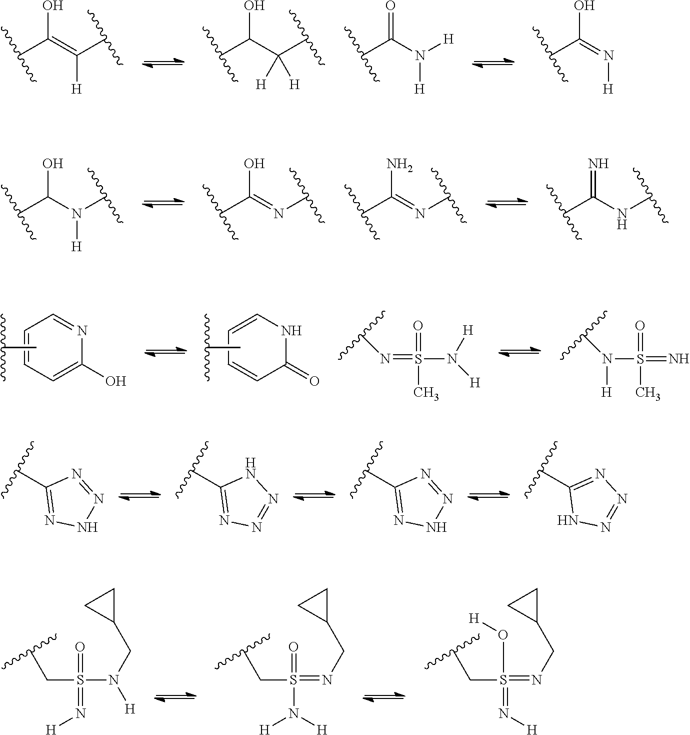

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (H), iodine-125 (¹²⁵I) or carbon-14 (14C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formula (I), (I-A), (II), (II-A), (II-B), or (II-C). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The methods and compositions of Formula (I), (I-A), (II), (II-A), (II-B), or (II-C) include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds of Formula (I), (I-A), (II), (II-A), (II-B), or (II-C), also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of compounds represented by Formula (I), (I-A), (II), (II-A), (II-B), or (II-C). The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

In certain embodiments, compounds or salts of Formula (I), (I-A), (II), (II-A), (II-B), or (II-C), may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

### Pharmaceutical Formulations

In some aspects, the present disclosure provides a pharmaceutical composition comprising a compound or salt of Formula (I), (I-A), (II), (II-A), (II-B), or (II-C) and at least one pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

A compound or salt of Formula (I), (I-A), (II), (II-A), (II-B), or (II-C) may be formulated in any suitable pharmaceutical formulation. A pharmaceutical formulation of the present disclosure typically contains an active ingredient (e.g., compound or salt of any one of Formula (I), (I-A), (II), (II-A), (II-B), or (II-C)), and one or more pharmaceutically acceptable excipients or carriers, including but not limited to: inert solid diluents and fillers, diluents, sterile aqueous solution and various organic solvents, permeation enhancers, antioxidents, solubilizers, and adjuvants.

Pharmaceutical formulations may be provided in any suitable form, which may depend on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments, the dosage form is formulated for oral administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

### Methods of Treatment

The compounds described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In some aspects, the present disclosure provides a method for treatment, comprising administering to a subject in need thereof an effective amount of a compound or salt of Formula (I), (I-A), (II), (II-A), (II-B), or (II-C). In some aspects, the present disclosure provides a method for treating cancer in a patient in need thereof, comprising administering to the subject an effective amount of a compound or salt of Formula (I), (I-A), (II), (II-A), (II-B), or (II-C).

In certain embodiments, the present disclosure can be used as a method of inhibiting an AKT1 protein in a subject in need thereof, comprising administering to the subject a compound or salt of Formula (I), (I-A), (II), (II-A), (II-B), or (II-C) or a pharmaceutical composition of Formula (I), (I-A), (II), (II-A), (II-B), or (II-C). In some embodiments, the AKT protein is a mutant AKT1 protein. In some embodiments, the mutant AKT1 protein comprises an E17K mutant.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### Example 1: N-(4-(2-(2-aminopyridin-3-yl)-5-(4-morpholinophenyl)-3H-imidazo[4,5-b]pyridin-3-yl)benzyl)-2-(4-formyl-3-hydroxyphenyl)acetamide

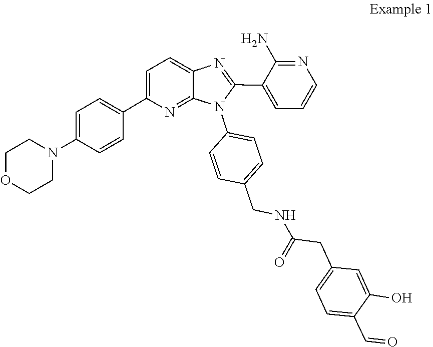 [SMILES: Nc1ncccc1-c1nc2ccc(-c3ccc(N4CCOCC4)cc3)nc2n1-c1ccc(CNC(=O)Cc2ccc(C=O)c(O)c2)cc1]

### Synthetic Route:

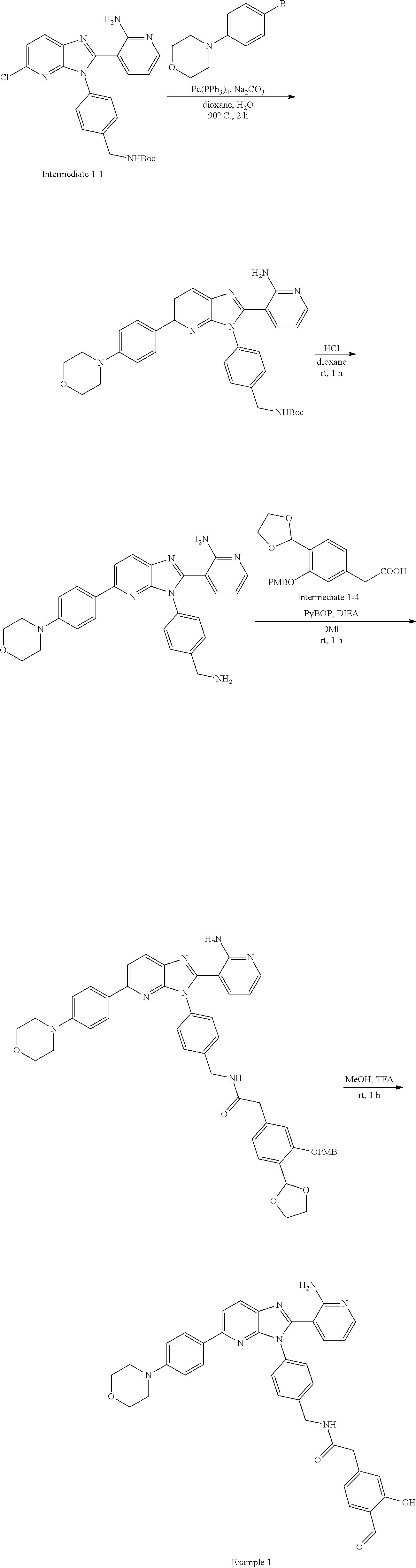

### Step 1: Synthesis of tert-butyl (4-(2-(2-aminopyridin-3-yl)-5-(4-morpholinophenyl)-3H-imidazo[4,5-b]pyridin-3-yl)benzyl)carbamate

To a solution of tert-butyl N-({4-[2-(2-aminopyridin-3-yl)-5-chloroimidazo[4,5-b]pyridin-3-yl]phenyl}methyl)carbamate (Intermediate 1-1) (300 mg, 0.665 mmol, 1 equiv) in 1,4-dioxane (4 mL) and water (1 mL) were added 4-(morpholin-4-yl)phenylboronic acid (207 mg, 0.998 mmol, 1.5 equiv), tetrakis(triphenylphosphine)palladium(0) (77 mg, 0.067 mmol, 0.1 equiv) and sodium carbonate (141 mg, 1.33 mmol, 2 equiv). The resulting mixture was stirred at 90° C. under nitrogen atmosphere for 2 h. The mixture was then concentrated in vacuo and the residue was purified by reverse-phase flash column chromatography on C18 silica gel using a 20-95% gradient of acetonitrile in water (+0.05% ammonium bicarbonate) with a 40 min hold at 70% acetonitrile to afford tert-butyl (4-(2-(2-aminopyridin-3-yl)-5-(4-morpholinophenyl)-3H-imidazo[4,5-b]pyridin-3-yl)benzyl)carbamate (360 mg, 94%) as a yellow solid. MS (ESI) calculated for C₃₃H₃₅N₇O₃: 577.28 m/z, found 578.55 [M+H]⁺.

### Step 2: Synthesis of 3-(3-(4-(aminomethyl)phenyl)-5-(4-morpholinophenyl)-3H-imidazo [4,5-b]pyridin-2-yl)pyridin-2-amine

A solution of tert-butyl N-({4-[2-(2-aminopyridin-3-yl)-5-[4-(morpholin-4-yl)phenyl]imidazo[4,5-b]pyridin-3-yl]phenyl}methyl)carbamate (416 mg, 0.720 mmol, 1 equiv) in 4N hydrochloric acid in 1,4-dioxane (10 mL) was stirred at room temperature for 1 h. The resulting solution was concentrated in vacuo to afford to afford 3-(3-(4-(aminomethyl)phenyl)-5-(4-morpholinophenyl)-3H-imidazo[4,5-b]pyridin-2-yl)pyridin-2-amine (340 mg, 99% crude) as a yellow solid, which was used in subsequent transformations without further purification. MS (ESI) calculated for C₂₈H₂₇N₇O: 477.23 m/z, found 478.25 [M+H]⁺.

### Step 3: Synthesis of 2-(4-(1,3-dioxolan-2-yl)-3-((4-methoxybenzyl)oxy)phenyl)-N-(4-(2-(2-aminopyridin-3-yl)-5-(4-morpholinophenyl)-3H-imidazo[4,5-b]pyridin-3-yl)benzyl)acetamide

To a solution of 3-(3-(4-(aminomethyl)phenyl)-5-(4-morpholinophenyl)-3H-imidazo[4,5-b]pyridin-2-yl)pyridin-2-amine (180 mg, 0.377 mmol, 1 equiv) in N,N-dimethylformamide (4 mL) were added N,N-diisopropylethylamine (244 mg, 1.89 mmol, 5 equiv), PyBOP (294 mg, 0.566 mmol, 1.5 equiv) and [4-(1,3-dioxolan-2-yl)-3-[(4-methoxyphenyl)methoxy]phenyl]acetic acid (Intermediate 1-4) (143 mg, 0.415 mmol, 1.1 equiv). The resulting mixture was stirred at room temperature for 1 h. The mixture was purified by reverse-phase flash column chromatography on C18 silica gel using a 20-95% gradient of acetonitrile in water (+0.05% ammonium bicarbonate) with a 40 minute hold at 70% acetonitrile to afford 2-(4-(1,3-dioxolan-2-yl)-3-((4-methoxybenzyl)oxy)phenyl)-N-(4-(2-(2-aminopyridin-3-yl)-5-(4-morpholinophenyl)-3H-imidazo[4,5-b]pyridin-3-yl)benzyl)acetamide (153 mg, 50%) as a yellow solid. MS (ESI) calculated for C₄₇H₄₅N₇O₆: 803.34 m/z, found 804.40 [M+H]⁺.

### Step 4: Synthesis of N-(4-(2-(2-aminopyridin-3-yl)-5-(4-morpholinophenyl)-3H-imidazo [4,5-b]pyridin-3-yl)benzyl)-2-(4-formyl-3-hydroxyphenyl)acetamide (Example 1)

A solution of 2-(4-(1,3-dioxolan-2-yl)-3-((4-methoxybenzyl)oxy)phenyl)-N-(4-(2-(2-aminopyridin-3-yl)-5-(4-morpholinophenyl)-3H-imidazo[4,5-b]pyridin-3-yl)benzyl)acetamide (100 mg, 0.124 mmol, 1 equiv) in 2,2,2-trifluoroacetic acid (0.6 mL) and methanesulfonic acid (0.2 mL) was stirred at room temperature for 1 h. The resulting mixture was concentrated in vacuo and purified by preparative HPLC on a XSelect CSH C18 OBD Column using a 12-28% gradient of acetonitrile in water (+0.1% formic acid) to afford N-(4-(2-(2-aminopyridin-3-yl)-5-(4-morpholinophenyl)-3H-imidazo[4,5-b]pyridin-3-yl)benzyl)-2-(4-formyl-3-hydroxyphenyl)acetamide (Example 1) (20 mg, 25%) as a yellow solid. MS (ESI) calculated for C₃₇H₃₃N₇O₄: 639.26 m/z, found 640.20 [M+H]⁺. ¹H NMR (300 MHz, DMSO-d₆) δ (ppm): 10.19 (s, 1H), 8.73-8.79 (m, 1H), 8.12-8.20 (m, 1H), 7.87-8.00 (m, 4H), 7.60-7.63 (m, 1H), 7.41-7.42 (m, 4H), 7.17-7.20 (m, 1H), 6.90-7.03 (m, 4H), 6.41-6.43 (m, 1H), 4.39-4.41 (m, 2H), 3.74-3.77 (m, 4H), 3.56 (s, 2H), 3.16-3.19 (m, 4H).

### Intermediate 1-1: tert-butyl (4-(2-(2-aminopyridin-3-yl)-5-chloro-3H-imidazo[4,5-b]pyridin-3-yl)benzyl)carbamate

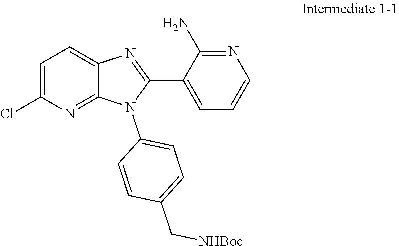 [SMILES: CC(C)(C)OC(=O)NCc1ccc(-n2c(-c3cccnc3N)nc3ccc(Cl)nc32)cc1]

### Synthetic Route:

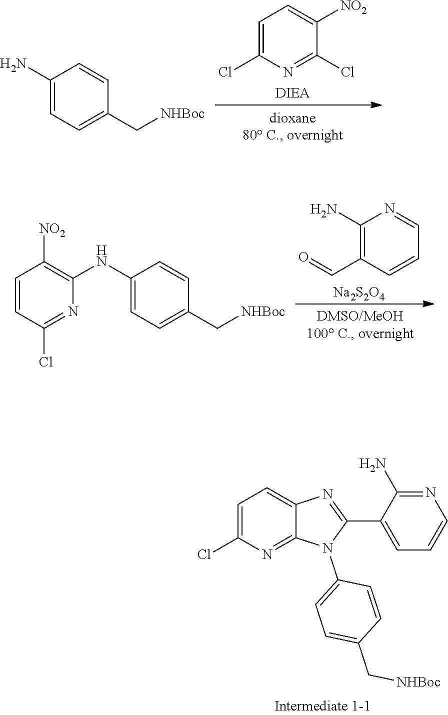

### Step 1: Synthesis of tert-butyl N-({4-[(6-chloro-3-nitropyridin-2-yl)amino]phenyl}methyl) carbamate

A solution of tert-butyl N-[(4-aminophenyl)methyl]carbamate (80 g, 360 mmol, 1 equiv), 2,6-dichloro-3-nitropyridine (70 g, 363 mmol, 1 equiv) and N,N-diisopropylethylamine (186 g, 1.44 mol, 4 equiv) in 1,4-dioxane (1.6 L) was stirred at 80° C. overnight. The mixture was allowed to cool to room temperature. The reaction was quenched with water (3 L) and extracted with ethyl acetate (1 L×3). The combined organic layers were washed with brine (1 L), dried over anhydrous sodium sulfate, filtered, and concentrated in vacuo. The crude product was re-crystallized from diethyl ether, petroleum ether (1:1, 1 L) to afford tert-butyl N-({4-[(6-chloro-3-nitropyridin-2-yl)amino]phenyl}methyl)carbamate (90 g, 66%) as a red solid. MS (ESI) calculated for C₁₇H₁₉ClN₄O₄: 378.11 m/z, found 401.05 [M+Na]⁺.

### Step 2: Synthesis of tert-butyl N-({4-[2-(2-aminopyridin-3-yl)-5-chloroimidazo[4,5-b]pyridin-3-yl]phenyl}methyl)carbamate (Intermediate 1-1)

A solution of tert-butyl N-({4-[(6-chloro-3-nitropyridin-2-yl)amino]phenyl}methyl)carbamate (50 g, 132 mmol, 1 equiv), 2-aminonicotinaldehyde (19.19 g, 158.4 mmol, 1.2 equiv) and sodium dithionite (54.93 g, 386.7 mmol, 2.93 equiv) in dimethyl sulfoxide (750 mL) and methanol (150 mL) was refluxed at 100° C. overnight. The reaction mixture was then poured into water (3 L) and the precipitate was collected by filtration. The solid was recrystallized from methanol/diethyl ether (1:4) to afford tert-butyl N-({4-[2-(2-aminopyridin-3-yl)-5-chloroimidazo[4,5-b]pyridin-3-yl]phenyl}methyl)carbamate (Intermediate 1-1) (15.5 g, 25%) as a yellow solid. MS (ESI) calculated for C₂₃H₂₃ClN₆O₂: 450.16 m/z, found 451.15 [M+H]⁺.

### Intermediate 1-4: 2-(4-(1,3-dioxolan-2-yl)-3-((4-methoxybenzyl)oxy)phenyl)acetic acid

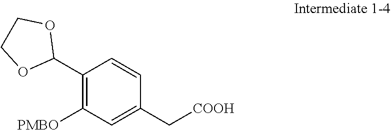

Synthetic route:

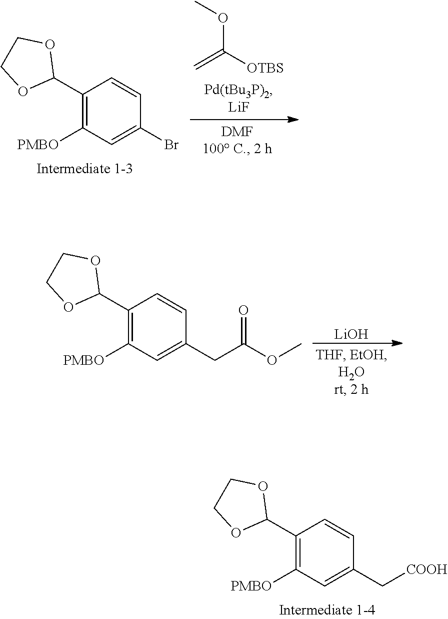

### Step 1: Synthesis of methyl 2-(4-(1,3-dioxolan-2-yl)-3-((4-methoxybenzyl)oxy)phenyl) acetate

To a solution of 2-(4-bromo-2-((4-methoxybenzyl)oxy)phenyl)-1,3-dioxolane (Intermediate 1-3) (4.15 g, 11.3 mmol, 1 equiv) and tert-butyldimethylsilyl methyl carbonate (8.65 g, 45.4 mmol, 4 equiv) in N,N-dimethylformamide (20 mL) were added bis(tri-tert-butylphosphine)palladium(0) (0.60 g, 1.14 mmol, 0.1 equiv) and lithium fluoride (0.6 g, 23 mmol, 2 equiv). The resulting mixture was stirred at 100° C. under nitrogen atmosphere for 2 h then cooled to 0° C. and quenched with water (100 mL). The resulting mixture was extracted with ethyl acetate (200 mL×3). The combined organic layers were washed with water (100 mL×3), dried over anhydrous sodium sulfate, filtered, and concentrated in vacuo. The residue was purified by silica gel column chromatography using a 0-60% gradient of ethyl acetate in petroleum ether to provide methyl 2-(4-(1,3-dioxolan-2-yl)-3-((4-methoxybenzyl)oxy)phenyl)acetate (3.0 g, 74%) as a light-yellow oil. MS (ESI) calculated for C₂₀H₂₂O₆: 358.14 m/z, found 359.10 [M+H]⁺.

### Step 2: Synthesis of 2-(4-(1,3-dioxolan-2-yl)-3-((4-methoxybenzyl)oxy)phenyl)acetic acid (Intermediate 1-4)

To a cooled (0° C.) solution of methyl 2-(4-(1,3-dioxolan-2-yl)-3-((4-methoxybenzyl)oxy)phenyl)acetate (3.0 g, 8.4 mmol, 1 equiv) in tetrahydrofuran (50 mL) and water (50 mL) was added a solution of lithium hydroxide (0.61 g, 25 mmol, 3 equiv) in water (12.5 mL) and the resulting mixture was stirred at room temperature for 2 h. The mixture was then concentrated in vacuo and suspended in water (5 mL). The pH of the mixture was brought to 6 with 2N hydrochloric acid and the resulting precipitate was collected by filtration and dried to provide 2-(4-(1,3-dioxolan-2-yl)-3-((4-methoxybenzyl)oxy)phenyl)acetic acid (Intermediate 1-4) (2.5 g, 87%) as a light-yellow solid. MS (ESI) calculated for C₁₉H₂₀O₆: 344.13 m/z, found 345.15 [M+H]⁺.

### Intermediate 1-3: 2-(4-bromo-2-((4-methoxybenzyl)oxy)phenyl)-1,3-dioxolane

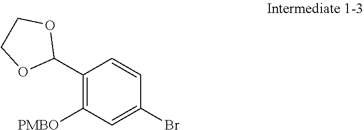

### Synthetic Route:

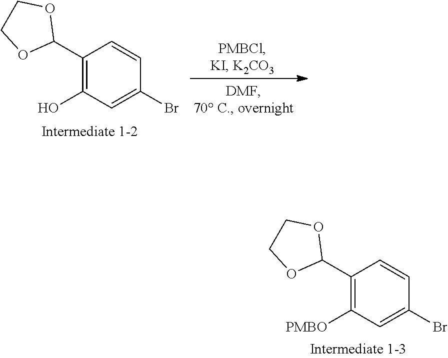

### Step 1: Synthesis of 2-(4-bromo-2-((4-methoxybenzyl)oxy)phenyl)-1,3-dioxolane (Intermediate 1-3)

To a solution of 5-bromo-2-(1,3-dioxolan-2-yl)phenol (Intermediate 1-2) (5.00 g, 20.4 mmol, 1 equiv) and p-methoxybenzyl chloride (3.83 g, 24.5 mmol, 1.2 equiv) in N,N-dimethylformamide (20 mL) were added potassium iodide (0.340 g, 2.04 mmol, 0.1 equiv) and potassium carbonate (8.46 g, 61.2 mmol, 3 equiv). After stirring overnight at 70° C. under nitrogen atmosphere the resulting mixture was partially concentrated in vacuo and quenched by addition of saturated aqueous ammonium chloride (20 mL) at 0° C. The resulting mixture was extracted with ethyl acetate (30 mL×3). The combined organic layers were washed with water (30 mL×3), dried over anhydrous sodium sulfate, filtered, and concentrated in vacuo. The resulting residue was purified by silica gel column chromatography using a 0-7% gradient of ethyl acetate in petroleum ether to provide 2-{4-bromo-2-[(4-methoxyphenyl)methoxy]phenyl}-1,3-dioxolane (Intermediate 1-3) (5.8 g, 78%) as a white solid. MS (ESI) calculated for C₁₇H₁₇BrO₄: 364.03 m/z, found 365.00 [M+H]⁺.

### Intermediate 1-2: 5-bromo-2-(1,3-dioxolan-2-yl)phenol

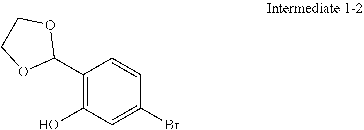 [SMILES: Oc1cc(Br)ccc1C1OCCO1]

### Synthetic Route:

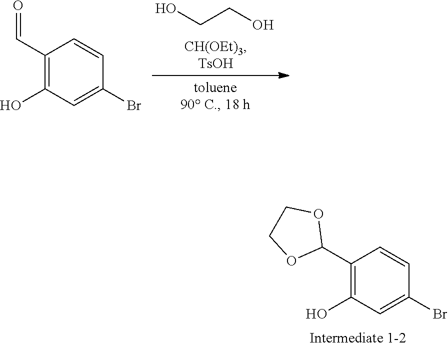

### Step 1: Synthesis of 5-bromo-2-(1,3-dioxolan-2-yl)phenol (Intermediate 1-2)

To a solution of 4-bromo-2-hydroxybenzaldehyde (10.0 g, 49.7 mmol, 1 equiv) in toluene (100 mL) were added p-toluenesulfonic acid (0.860 g, 4.98 mmol, 0.1 equiv), ethylene glycol (15.44 g, 248.7 mmol, 5 equiv) and triethyl orthoformate (22.12 g, 149.24 mmol, 3 equiv) at room temperature. The resulting solution was stirred at room temperature for 10 min then overnight at 90° C. The solution was cooled to 0° C. and quenched by addition of saturated aqueous ammonium chloride (80 mL). The resulting mixture was extracted with ethyl acetate (100 mL×3). The combined organic layers were washed with water (100 mL×3), dried over anhydrous sodium sulfate, filtered, and concentrated in vacuo. The residue was purified by silica gel column chromatography using a 0-50% gradient of ethyl acetate in petroleum ether to provide 5-bromo-2-(1,3-dioxolan-2-yl)phenol (Intermediate 1-2) (6 g, 49%) as a light-yellow oil. MS (ESI) calculated for C₉H₉BrO₃: 244.97 m/z, found 245.95 [M+H]⁺.

### Example 2: N-(4-(5-(3-acetamidophenyl)-2-(2-aminopyridin-3-yl)-3H-imidazo[4,5-b]pyridin-3-yl)benzyl)-2-(4-formyl-3-hydroxyphenyl)acetamide

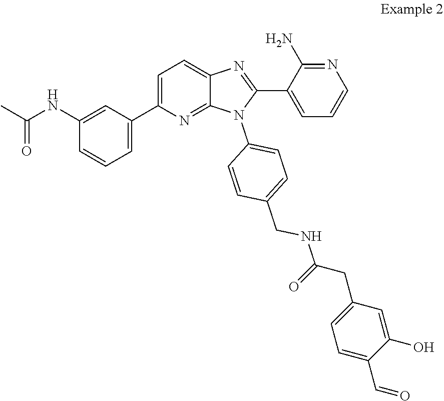 [SMILES: CC(=O)Nc1cccc(-c2ccc3nc(-c4cccnc4N)n(-c4ccc(CNC(=O)Cc5ccc(C=O)c(O)c5)cc4)c3n2)c1]

Example 2 was prepared in a manner analogous to Example 1 using 3-acetamidophenylboronic acid in place of 4-(morpholin-4-yl)phenylboronic acid. MS (ESI) calculated for C₃₅H₂₉N₇O₄: 611.23 m/z, found 612.15 [M+H]⁺. ¹H NMR (400 MHz, DMSO-d₆) δ (ppm): δ 10.17 (s, 1H), 8.30-8.37 (m, 1H), 8.11-8.18 (m, 1H), 8.03-8.07 (m, 1H), 7.88-7.95 (m, 1H), 7.64-7.73 (m, 3H), 7.58-7.63 (m, 1H), 7.47-7.52 (m, 2H), 7.38-7.44 (m, 3H), 6.94-6.98 (m, 1H), 6.88-6.93 (m, 1H), 6.70-6.79 (m, 1H), 4.39 (s, 2H), 3.55 (s, 2H), 2.06 (s, 3H).

### Example 3: N-(4-(2-(2-aminopyridin-3-yl)-5-morpholino-3H-imidazo[4,5-b]pyridin-3-yl)benzyl)-3-formyl-4-hydroxybenzamide

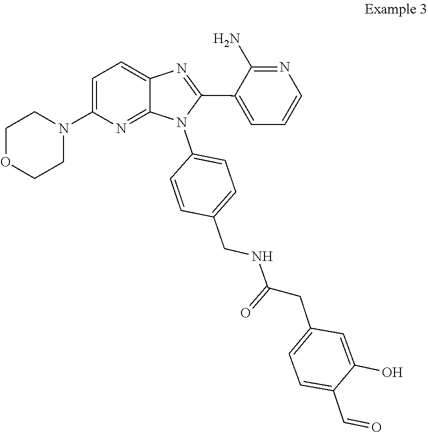 [SMILES: Nc1ncccc1-c1nc2ccc(N3CCOCC3)nc2n1-c1ccc(CNC(=O)Cc2ccc(C=O)c(O)c2)cc1]

Example 3 was prepared in a manner analogous to Example 1 (starting from step 3) using Intermediate 3-1 in place of the amine starting material and Intermediate 3-4 in place of Intermediate 1-4. MS (ESI) calculated for C₃₀H₂₇N₇O₄: 549.21 m/z, found 550.15 [M+H]⁺. ¹H NMR (400 MHz, DMSO-d₆) δ 10.32 (s, 1H), 8.27-8.28 (m, 1H), 7.99-8.09 (m, 3H), 7.52-7.55 (m, 1H), 7.39-7.45 (m, 4H), 7.08-7.10 (m, 1H), 6.95-6.97 (m, 1H), 6.71-6.74 (m, 1H), 4.55 (s, 2H), 3.66-3.68 (m, 4H), 3.37-3.42 (m, 4H).

### Intermediate 3-1: 3-(3-(4-(aminomethyl)phenyl)-5-morpholino-3H-imidazo[4,5-b]pyridin-2-yl)pyridin-2-amine

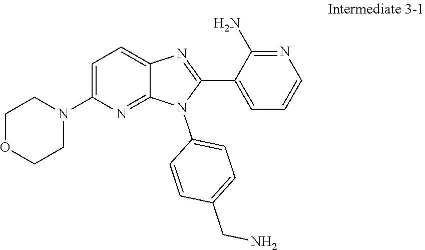 [SMILES: NCc1ccc(-n2c(-c3cccnc3N)nc3ccc(N4CCOCC4)nc32)cc1]

### Synthetic Route:

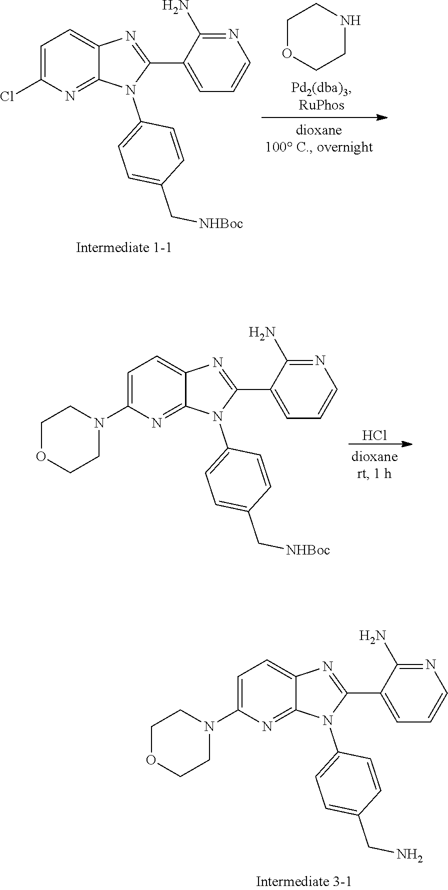

### Step 1: Synthesis of tert-butyl N-({4-[2-(2-aminopyridin-3-yl)-5-(morpholin-4-yl)imidazo [4,5-b]pyridin-3-yl]phenyl}methyl)carbamate

To a solution of tert-butyl N-({4-[2-(2-aminopyridin-3-yl)-5-chloroimidazo[4,5-b]pyridin-3-yl]phenyl}methyl)carbamate (Intermediate 1-1) (1.00 g, 2.22 mmol, 1 equiv) in 1,4-dioxane (10 mL) were added morpholine (0.970 g, 11.1 mmol, 5 equiv), tris(dibenzylideneacetone)dipalladium(0) (0.20 g, 0.22 mmol, 0.1 equiv) and RuPhos (0.21 g, 0.44 mmol, 0.2 equiv). The resulting mixture was stirred at 100° C. under nitrogen atmosphere overnight then cooled to room temperature and quenched with water (100 mL). The resulting mixture was extracted with ethyl acetate (3×100 mL). The combined organic layers were dried over sodium sulfate, filtered, and concentrated in vacuo. The resulting residue was purified by reverse-phase flash column chromatography on C18 silica gel using a 20-95% gradient of acetonitrile in water (+0.05% ammonium bicarbonate) with a 10 minute hold at 70% acetonitrile to afford tert-butyl N-({4-[2-(2-aminopyridin-3-yl)-5-(morpholin-4-yl)imidazo[4,5-b]pyridin-3-yl]phenyl}methyl)carbamate (400 mg, 36%) as a yellow solid. MS (ESI) calculated for C₂₇H₃₁N₇O₃: 501.25 m/z, found 502.45 [M+H]⁺.

### Step 2: Synthesis of 3-{3-[4-(aminomethyl)phenyl]-5-(morpholin-4-yl)imidazo[4,5-b]pyridin-2-yl}pyridin-2-amine (Intermediate 3-1)

A solution of tert-butyl N-({4-[2-(2-aminopyridin-3-yl)-5-(morpholin-4-yl)imidazo[4,5-b]pyridin-3-yl]phenyl}methyl)carbamate (400 mg, 0.797 mmol, 1 equiv) in 4N hydrochloric acid in 1,4-dioxane (20 mL) was stirred at room temperature for 1 h. The resulting solution was concentrated in vacuo to afford 3-{3-[4-(aminomethyl)phenyl]-5-(morpholin-4-yl)imidazo[4,5-b]pyridin-2-yl}pyridin-2-amine (Intermediate 3-1) (300 mg, 94%) as a yellow solid, which was used without purification in subsequent transformations. MS (ESI) calculated for C₂₂H₂₃N₇O: 401.20 m/z, found 402.25 [M+H]⁺.

### Intermediate 3-4: 3-(1,3-dioxolan-2-yl)-4-((4-methoxybenzyl)oxy)benzoic acid

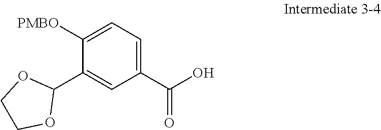

### Synthetic Route:

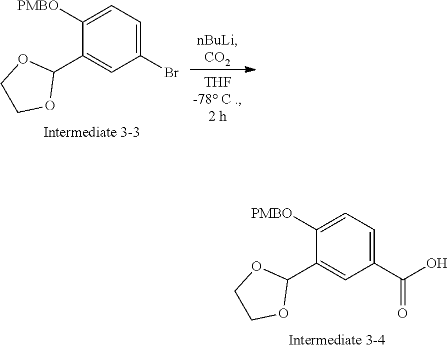

### Step 1: Synthesis of 3-(1,3-dioxolan-2-yl)-4-((4-methoxybenzyl)oxy)benzoic acid (Intermediate 3-4)

n-Butyllithium (3.3 mL, 8.2 mmol, 3 equiv) was added dropwise to a cooled (−78° C.) solution of 2-(5-bromo-2-((4-methoxybenzyl)oxy)phenyl)-1,3-dioxolane (Intermediate 3-3) (1.00 g, 2.74 mmol, 1 equiv) in tetrahydrofuran (20 mL) and the resulting solution was stirred at −78° C. for 2 h. Carbon dioxide was then passed through the solution using a needle for 10 min. The reaction was quenched with saturated aqueous ammonium chloride (10 mL) and the mixture was extracted with ethyl acetate (20 mL×3). The combined organic phases were washed with brine (30 mL), dried over sodium sulfate, filtered, and concentrated in vacuo. The resulting residue was purified by reverse-phase column chromatography on C18 silica gel using a 5-45% gradient of acetonitrile in water (+0.05% ammonium bicarbonate) to provide 3-(1,3-dioxolan-2-yl)-4-((4-methoxybenzyl)oxy)benzoic acid (Intermediate 3-4) (400 mg, 44%) as a light-yellow oil. MS (ESI) calculated for C₁₈H₁₈O₆: 330.11 m/z, found 329.05 [M−H]⁻.

### Intermediate 3-3: 2-{5-bromo-2-[(4-methoxyphenyl)methoxy]phenyl}-1,3-dioxolane

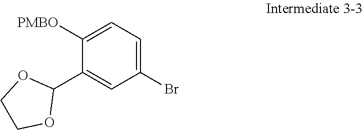

Intermediate 3-3 was prepared in a manner analogous to Intermediate 1-3 using Intermediate 3-2 in place of Intermediate 1-2. MS (ESI) calculated for C₁₇H₁₇BrO₄: 364.03 m/z, found 366.95, 388.95 [M+Na, M+Na+2]⁺.

### Intermediate 3-2: 4-bromo-2-(1,3-dioxolan-2-yl)phenol

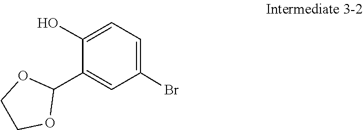 [SMILES: Oc1ccc(Br)cc1C1OCCO1]

Intermediate 3-2 was prepared in a manner analogous to Intermediate 1-2 using 5-bromo-2-hydroxybenzaldehyde in place of 4-bromo-2-hydroxybenzaldehyde. MS (ESI) calculated for C₉H₉BrO₃: 243.97 m/z, found 244.90, 246.90 [M+H, M+H+2]⁺.

### Example 4: N-({4-[2-(2-aminopyridin-3-yl)-5-phenylimidazo[4,5-b]pyridin-3-yl]phenyl}methyl)-2-fluoro-5-formyl-4-hydroxybenzamide

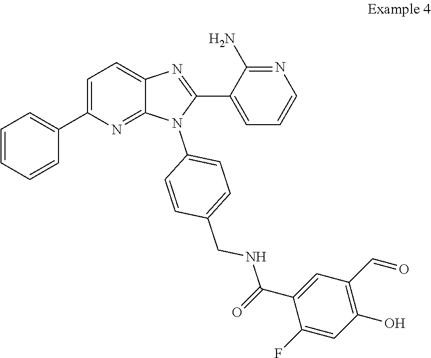 [SMILES: Nc1ncccc1-c1nc2ccc(-c3ccccc3)nc2n1-c1ccc(CNC(=O)c2cc(C=O)c(O)cc2F)cc1]

Example 4 was prepared in a manner analogous to Example 1 (starting from step 3) using Intermediate 4-1 in place of the amine starting material and Intermediate 4-3 in place of Intermediate 1-4. MS (ESI) calculated for C₃₂H₂₃FN₆O₃: 558.18 m/z, found 559.15[M+H]⁺. ¹H NMR (400 MHz, DMSO-d₆) δ (ppm): 10.18 (s, 1H), 8.23-8.25 (m, 1H), 8.05-8.13 (m, 1H), 7.95-8.00 (m, 4H), 7.45-7.52 (m, 6H), 7.43-7.45 (m, 1H), 7.36-7.40 (m, 1H), 6.81-6.84 (m, 1H), 6.42-6.45 (m, 1H), 4.56 (s, 2H). ¹⁹F-NMR (400 MHz, DMSO-d₆) δ (ppm): −101.75.

### Intermediate 4-1: 3-(3-(4-(aminomethyl)phenyl)-5-phenyl-3H-imidazo[4,5-b]pyridin-2-yl) pyridin-2-amine

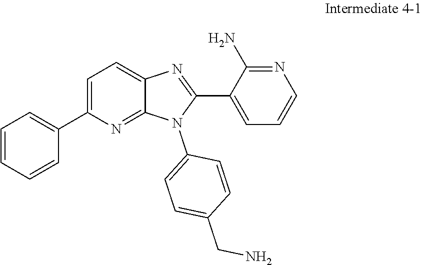 [SMILES: NCc1ccc(-n2c(-c3cccnc3N)nc3ccc(-c4ccccc4)nc32)cc1]

### Synthetic Route:

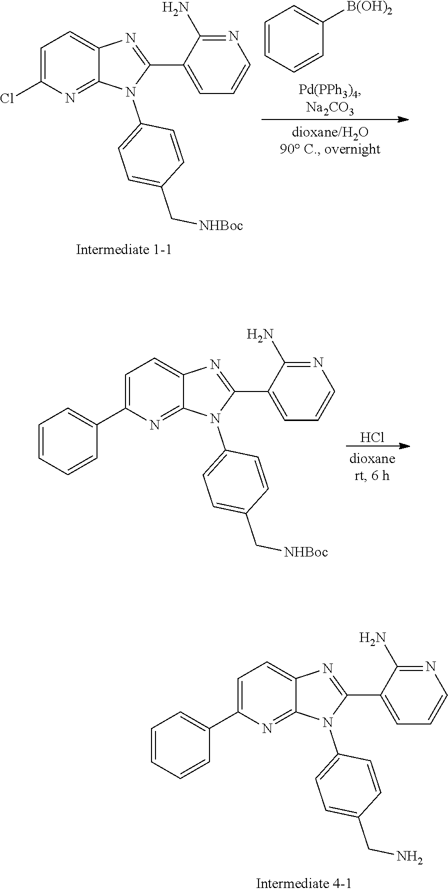

### Step 1: Synthesis of tert-butyl (4-(2-(2-aminopyridin-3-yl)-5-phenyl-3H-imidazo[4,5-b]pyridin-3-yl)benzyl)carbamate

A suspension of tert-butyl N-({4-[2-(2-aminopyridin-3-yl)-5-chloroimidazo[4,5-b]pyridin-3-yl]phenyl}methyl)carbamate (Intermediate 1-1) (40 g, 89 mmol, 1 equiv), phenyl boronic acid (21.63 g, 177.4 mmol, 2 equiv), tetrakis(triphenylphosphine)palladium(0) (10.25 g, 8.871 mmol, 0.1 equiv) and sodium carbonate (18.80 g, 177.4 mmol, 2 equiv) in 1,4-dioxane (400 mL) and water (100 mL) was stirred at 90° C. overnight under nitrogen atmosphere. The reaction mixture was cooled to room temperature and poured into water (2 L). The precipitate was filtered and dried in an oven. The obtained solid was recrystallized from methanol/diethyl ether (1:4) to afford tert-butyl N-({4-[2-(2-aminopyridin-3-yl)-5-phenylimidazo[4,5-b]pyridin-3-yl]phenyl}methyl)carbamate as an orange solid (30 g, 69%). MS (ESI) calculated for C₂₉H₂₈N₆O₂: 492.23 m/z, found 493.25 [M+H]⁺.

### Step 2: Synthesis of 3-(3-(4-(aminomethyl)phenyl)-5-phenyl-3H-imidazo[4,5-b]pyridin-2-yl)pyridin-2-amine (Intermediate 4-1)

To a solution of tert-butyl N-({4-[2-(2-aminopyridin-3-yl)-5-phenylimidazo[4,5-b]pyridin-3-yl]phenyl}methyl)carbamate (20 g, 41 mmol, 1 equiv) in dichloromethane (200 mL) was added hydrochloric acid (4N in dioxane, 100 mL). The resulting mixture was stirred at room temperature for 6 h and concentrated in vacuo. The resulting residue was recrystallized from diethyl ether to afford 3-{3-[4-(aminomethyl)phenyl]-5-phenylimidazo[4,5-b]pyridin-2-yl}pyridin-2-amine (Intermediate 4-1) as an orange solid (13 g, 82%). MS (ESI) calculated for C₂₄H₂₀N₆: 392.17 m/z, found 393.25 [M+H]⁺.

### Intermediate 4-3: 5-(1,3-dioxolan-2-yl)-2-fluoro-4-((4-methoxybenzyl)oxy)benzoic acid

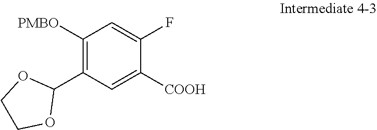

Intermediate 4-3 was prepared in a manner analogous to Intermediate 3-4 using Intermediate 4-2 in place of Intermediate 3-3. MS (ESI) calculated for C₁₈H₁₇FO₆: 348.10 m/z, found 347.10 [M−H]⁻.

### Intermediate 4-2: 2-(5-bromo-4-fluoro-2-((4-methoxybenzyl)oxy)phenyl)-1,3-dioxolane

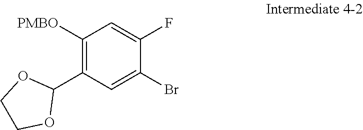

Intermediate 4-2 was prepared in a manner analogous to Intermediate 1-3 (via Intermediate 1-2) starting from 5-bromo-4-fluoro-2-hydroxybenzaldehyde in place of 5-bromo-2-hydroxybenzaldehyde. MS (ESI) calculated for C₁₇H₁₆BrFO₄: 382.02 m/z, found 383.05 [M+H]⁺.

### Example 5: N-(4-(2-(2-aminopyridin-3-yl)-5-phenyl-3H-imidazo[4,5-b]pyridin-3-yl)benzyl)

### 2-(4-formyl-3-hydroxy-2-(trifluoromethyl)phenyl)acetamide

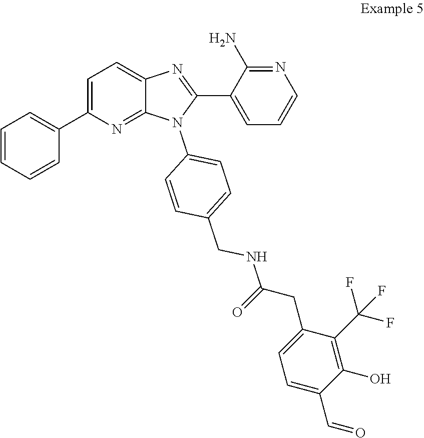 [SMILES: Nc1ncccc1-c1nc2ccc(-c3ccccc3)nc2n1-c1ccc(CNC(=O)Cc2ccc(C=O)c(O)c2C(F)(F)F)cc1]

Example 5 was prepared in a manner analogous to Example 1 (starting from Step 3) using Intermediate 4-1 in place of the amine starting material and Intermediate 5-2 in place of Intermediate 1-4. MS (ESI) calculated for C₃₄H₂₅F₃N₆O₃: 622.19 m/z, found 623.15 [M+H]⁺. ¹H NMR (400 MHz, DMSO-d₆) δ 11.86 (s, 1H), 10.06 (s, 1H), 8.64 (s, 1H), 8.33 (s, 1H), 7.99-8.17 (m, 4H), 7.96-7.98 (m, 1H), 7.60-7.72 (m, 1H), 7.28-7.59 (m, 7H), 7.09-7.20 (m, 1H), 6.60-6.77 (m, 1H), 4.41 (s, 2H), 3.90 (s, 2H). ¹⁹F NMR (282 MHz, DMSO-d₆) δ (ppm): −52.68.

### Intermediate 5-2: 2-(4-(1,3-dioxolan-2-yl)-3-((4-methoxybenzyl)oxy)-2-(trifluoromethyl) phenyl)acetic acid

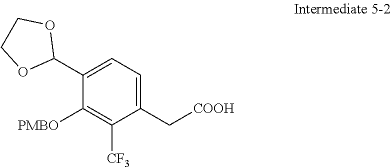

Intermediate 5-2 was prepared in a manner analogous to Intermediate 1-4 (via Intermediates 1-3 and 1-2) using Intermediate 5-1 in place of 4-bromo-2-hydroxybenzaldehyde. MS (ESI) calculated for C₂₀H₁₉F₃O₆: 412.11 m/z, found 367.20 [M−H]⁻ (mass of the aldehyde resulting from loss of the ethylene glycol protecting group).

### Intermediate 5-1: 4-bromo-2-hydroxy-3-(trifluoromethyl)benzaldehyde

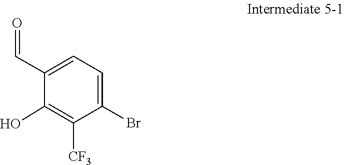 [SMILES: O=Cc1ccc(Br)c(C(F)(F)F)c1O]

### Synthetic Route:

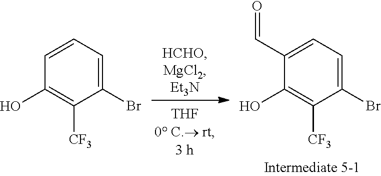 [SMILES: Oc1cccc(Br)c1C(F)(F)F ; O=Cc1ccc(Br)c(C(F)(F)F)c1O]

### Step 1: Synthesis of 4-bromo-2-hydroxy-3-(trifluoromethyl)benzaldehyde (Intermediate 5-1)

A mixture of 3-bromo-2-(trifluoromethyl)phenol (3.00 g, 12.5 mmol, 1 equiv), magnesium(II) chloride (1.78 g, 18.7 mmol, 1.5 equiv), triethylamine (5.67 g, 56 mmol, 4.5 equiv) and paraformaldehyde (8.97 g, 99.6 mmol, 8 equiv) in tetrahydrofuran (30 mL) was stirred for 2 days at 60° C. The resulting mixture was diluted with water (30 mL) and the resulting mixture was extracted with ethyl acetate (3×30 mL). The combined organic layers were washed with water (2×20 mL), dried over anhydrous sodium sulfate, filtered, and concentrated in vacuo. The resulting residue was purified by silica gel column chromatography using a 0-30% gradient of ethyl acetate in petroleum ether to afford 4-bromo-2-hydroxy-3-(trifluoromethyl)benzaldehyde (Intermediate 5-1) (1.1 g, 33%) as a white solid. MS (ESI) calculated for C₈H₄BrF₃O₂: 267.93 m/z, found 266.90, 268.90 [M−H, M+2−H]⁻.

### Example 6: N-({4-[2-(2-aminopyridin-3-yl)-5-[2-(morpholine-4-carbonyl)cyclopropyl]imidazo[4,5-b]pyridin-3-yl]phenyl}methyl)-2-(4-formyl-3-hydroxyphenyl)acetamide

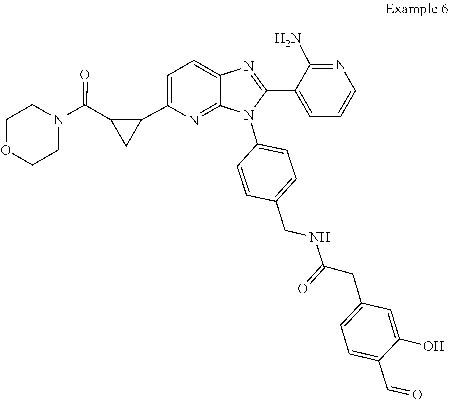 [SMILES: Nc1ncccc1-c1nc2ccc(C3CC3C(=O)N3CCOCC3)nc2n1-c1ccc(CNC(=O)Cc2ccc(C=O)c(O)c2)cc1]

Example 6 was prepared in a manner analogous to Example 1 (starting from Step 3) using Intermediate 6-1 in place of the amine starting material. MS (ESI) calculated for C₃₅H₃₃N₇O₅: 631.25 m/z, found 632.30 [M+H]⁺. ¹H-NMR (400 MHz, DMSO-d₆) δ (ppm): 10.20 (s, 1H), 8.08-8.12 (m, 1H), 7.95-8.01 (m, 1H), 7.56-7.62 (m, 1H), 7.32-7.40 (m, 5H), 7.09-7.15 (m, 1H), 6.95 (s, 1H), 6.86-6.91 (m, 1H), 6.35-6.41 (m, 1H), 4.34-4.39 (m, 2H), 3.35-3.59 (m, 10H), 2.68 (s, 1H), 2.51 (s, 1H), 2.23-2.35 (m, 1H), 1.41-1.49 (m, 1H), 1.33-1.40 (m, 1H).

### Intermediate 6-1: 3-{3-[4-(aminomethyl)phenyl]-5-[2-(morpholine-4-carbonyl)cyclopropyl]imidazo[4,5-b]pyridin-2-yl}pyridin-2-amine

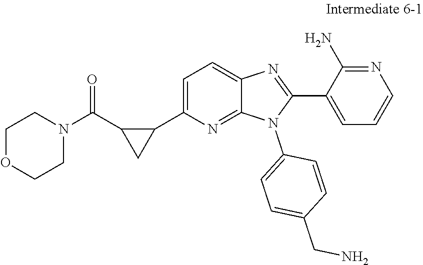 [SMILES: NCc1ccc(-n2c(-c3cccnc3N)nc3ccc(C4CC4C(=O)N4CCOCC4)nc32)cc1]

### Synthetic Route:

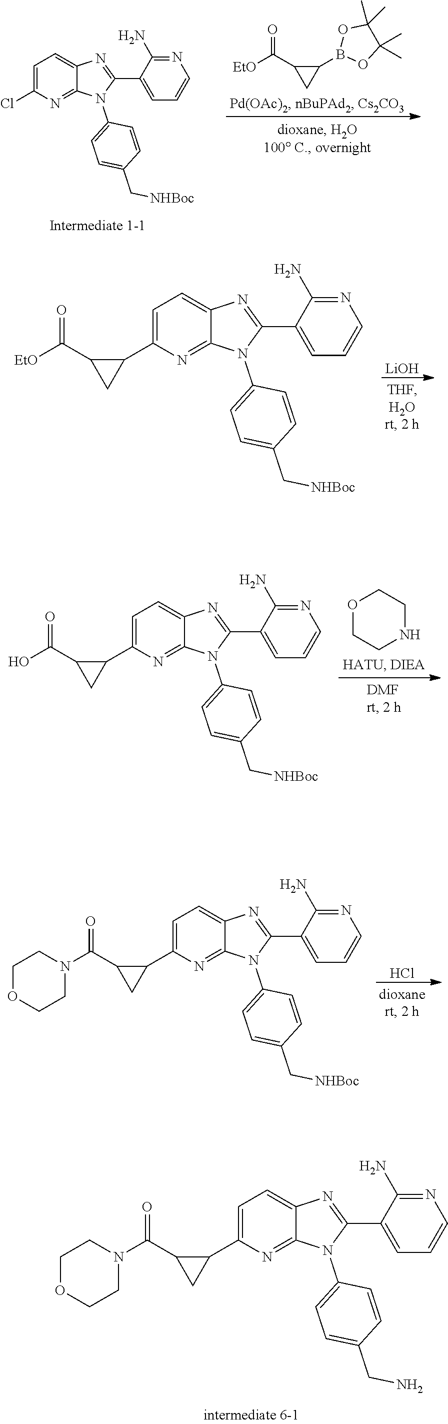

### Step 1: Synthesis of ethyl 2-[2-(2-aminopyridin-3-yl)-3-(4-{[(tert-butoxycarbonyl) amino]methyl}phenyl)imidazo[4,5-b]pyridin-5-yl]cyclopropane-1-carboxylate

To a solution of tert-butyl N-({4-[2-(2-aminopyridin-3-yl)-5-chloroimidazo[4,5-b]pyridin-3-yl]phenyl}methyl)carbamate (Intermediate 1-1) (1.00 g, 2.22 mmol, 1 equiv) in 1,4-dioxane (12 mL) and water (3 mL) were added ethyl 2-(4,4,5,5-tetramethyl-1,3,2-dioxaborolan-2-yl)cyclopropane-1-carboxylate (0.64 g, 2.7 mmol, 1.2 equiv), palladium(II) acetate (0.05 g, 0.22 mmol, 0.1 equiv), cesium carbonate (2.17 g, 6.65 mmol, 3 equiv) and bis(adamantan-1-yl)(butyl)phosphanium tetrafluoroborate (0.10 g, 0.22 mmol, 0.1 equiv). The resulting mixture was stirred at 100° C. overnight under nitrogen atmosphere. The mixture was then cooled to room temperature and quenched by addition of water (50 mL). The resulting mixture was extracted with ethyl acetate (3×50 mL). The combined organic layers were dried over anhydrous sodium sulfate, filtered, and concentrated in vacuo. The resulting residue was purified by silica gel column chromatography using a 0-95% gradient of ethyl acetate in petroleum with a 40-minute hold at 50% to provide ethyl 2-[2-(2-aminopyridin-3-yl)-3-(4-{[(tert-butoxycarbonyl)amino]methyl}phenyl)imidazo[4,5-b]pyridin-5-yl]cyclopropane-1-carboxylate (390 mg, 30%) as a yellow solid. MS (ESI) calculated for C₂₉H₃₂N₆O₄: 528.61 m/z, found 529.35 [M+H]⁺.

### Step 2: Synthesis of 2-[2-(2-aminopyridin-3-yl)-3-(4-{[(tert-butoxycarbonyl)amino]methyl}phenyl) imidazo[4,5-b]pyridin-5-yl]cyclopropane-1-carboxylic acid

To a solution of ethyl 2-[2-(2-aminopyridin-3-yl)-3-(4-{[(tert-butoxycarbonyl) amino]methyl}phenyl)imidazo[4,5-b]pyridin-5-yl]cyclopropane-1-carboxylate (390 mg, 0.738 mmol, 1 equiv) in tetrahydrofuran (5 mL) and water (5 mL) was added 2N aqueous lithium hydroxide (1.1 mL, 3 equiv). The resulting mixture was stirred at room temperature for 2 h. The mixture was concentrated in vacuo and the residue was purified by reverse-phase column chromatography on C18 silica gel using a 0-95% gradient of acetonitrile in water (+0.05% ammonium bicarbonate) to afford 2-[2-(2-aminopyridin-3-yl)-3-(4-{[(tert-butoxycarbonyl)amino]methyl}phenyl)imidazo[4,5-b]pyridin-5-yl]cyclopropane-1-carboxylic acid (289 mg, 70%) as a yellow solid. MS (ESI) calculated for C₂₇H₂₈N₆O₄: 500.56 m/z, found 501.25 [M+H]⁺.

### Step 3: Synthesis of tert-butyl N-({4-[2-(2-aminopyridin-3-yl)-5-[2-(morpholine-4-carbonyl) cyclopropyl]imidazo[4,5-b]pyridin-3-yl]phenyl}methyl)carbamate

To a solution of 2-[2-(2-aminopyridin-3-yl)-3-(4-{[(tert-butoxycarbonyl)amino]methyl}phenyl)imidazo[4,5-b]pyridin-5-yl]cyclopropane-1-carboxylic acid (280 mg, 0.559 mmol, 1 equiv) in N,N-dimethylformamide (5 mL) was added N,N-diisopropylethylamine (217 mg, 1.68 mmol, 3 equiv), HATU (319 mg, 0.839 mmol, 1.5 equiv) and morpholine (73 mg, 0.84 mmol, 1.5 equiv). The resulting mixture was stirred at room temperature for 2 h. The mixture was purified by reverse-phase flash column chromatography on C18 silica gel using a 0-95% gradient of acetonitrile in water (+0.05% ammonium bicarbonate) with a 10 minute hold at 50% acetonitrile to afford tert-butyl N-({4-[2-(2-aminopyridin-3-yl)-5-[2-(morpholine-4-carbonyl)cyclopropyl]imidazo[4,5-b]pyridin-3-yl]phenyl}methyl)carbamate (236 mg, 70%) as a yellow solid. MS (ESI) calculated for C₃₁H₃₅N₇O₄: 569.28 m/z, found 570.35 [M+H]⁺.

### Step 4: Synthesis of 3-{3-[4-(aminomethyl)phenyl]-5-[2-(morpholine-4-carbonyl)cyclopropyl]imidazo[4,5-b]pyridin-2-yl}pyridin-2-amine (Intermediate 6-1)

A mixture of tert-butyl N-({4-[2-(2-aminopyridin-3-yl)-5-[2-(morpholine-4-carbonyl)cyclopropyl]imidazo[4,5-b]pyridin-3-yl]phenyl}methyl)carbamate (230 mg, 0.404 mmol, 1 equiv) in 4N hydrochloric acid in 1,4-dioxane (5 mL) was stirred at room temperature for 2 h. The mixture was concentrated in vacuo to afford 3-{3-[4-(aminomethyl)phenyl]-5-[2-(morpholine-4-carbonyl)cyclopropyl]imidazo[4,5-b]pyridin-2-yl}pyridin-2-amine (Intermediate 6-1) (182 mg, 91%) as a yellow solid. MS (ESI) calculated for C₂₆H₂₇N₇O₂: 469.22 m/z, found 470.25 [M+H]⁺.

### Example 7: N-({4-[2-(2-aminopyridin-3-yl)-5-(difluoromethyl)imidazo[4,5-b]pyridin-3-yl]phenyl}methyl)-2-(4-formyl-3-hydroxyphenyl)acetamide

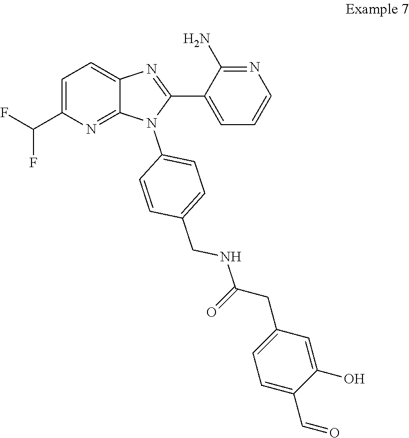 [SMILES: Nc1ncccc1-c1nc2ccc(C(F)F)nc2n1-c1ccc(CNC(=O)Cc2ccc(C=O)c(O)c2)cc1]

Example 7 was prepared in a manner analogous to Example 1 (starting from Step 3) using Intermediate 7-1 in place of the amine starting material. MS (ESI) calculated for C₂₈H₂₂F₂N₆O₃: 528.17 m/z, found 529.10 [M+H]⁺. ¹H NMR (400 MHz, DMSO-d₆) δ 10.72 (s, 1H), 10.21 (s, 1H), 8.72 (s, 1H), 8.43 (s, 1H), 8.01-8.16 (m, 1H), 7.53-7.58 (m, 4H), 7.36-7.52 (m, 4H), 7.29-7.07 (m, 1H), 7.06-6.84 (m, 2H), 6.69 (s, 1H), 4.37 (s, 2H), 3.54 (s, 2H). ¹⁹F NMR (282 MHz, DMSO-d₆) δ (ppm): −112.04.

### Intermediate 7-1: 3-(3-(4-(aminomethyl)phenyl)-5-(difluoromethyl)-3H-imidazo[4,5-b]pyridin-2-yl)pyridin-2-amine

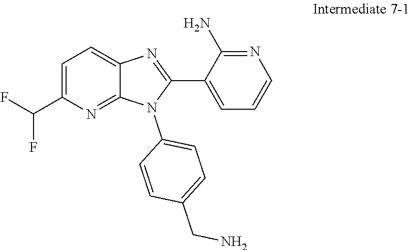 [SMILES: NCc1ccc(-n2c(-c3cccnc3N)nc3ccc(C(F)F)nc32)cc1]

### Synthetic Route:

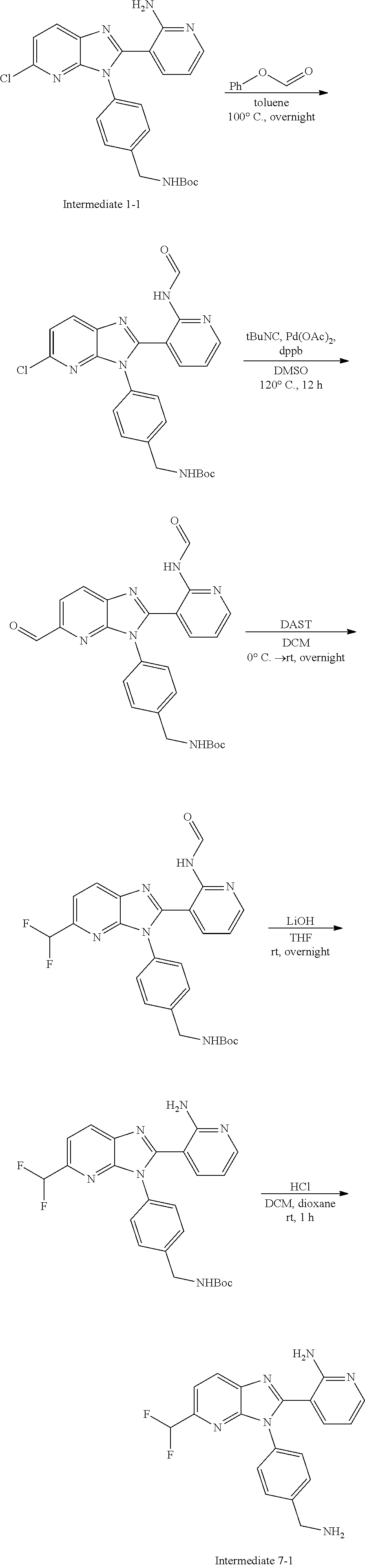

### Step 1: Synthesis of tert-butyl (4-(5-chloro-2-(2-formamidopyridin-3-yl)-3H-imidazo[4,5-b]pyridin-3-yl)benzyl)carbamate

A mixture of tert-butyl N-({4-[2-(2-aminopyridin-3-yl)-5-chloroimidazo[4,5-b]pyridin-3-yl]phenyl}methyl)carbamate (Intermediate 1-1) (5.00 g, 6.65 mmol, 1 equiv) and phenyl formate (2.12 g, 20.0 mmol, 3 equiv) in toluene was stirred for 12 h at 100° C. under nitrogen atmosphere. The resulting mixture was concentrated under reduced pressure and purified by silica gel column chromatography using a 0-20% gradient of ethyl acetate in dichloromethane to afford tert-butyl (4-(5-chloro-2-(2-formamidopyridin-3-yl)-3H-imidazo[4,5-b]pyridin-3-yl)benzyl)carbamate (2 g, 63%) as a yellow solid. MS (ESI) calculated for C₂₄H₂₃ClN₆O₃: 478.15 m/z, found 479.10 [M+H]⁺.

### Step 2: Synthesis of tert-butyl (4-(2-(2-formamidopyridin-3-yl)-5-formyl-3H-imidazo[4,5-b]pyridin-3-yl)benzyl)carbamate

A mixture of tert-butyl N-({4-[5-chloro-2-(2-formamidopyridin-3-yl)imidazo[4,5-b]pyridin-3-yl]phenyl}methyl)carbamate (1.00 g, 2.09 mmol, 1 equiv), palladium(II) acetate (0.05 g, 0.21 mmol, 0.1 equiv) and [4-(diphenylphosphanyl)butyl]diphenylphosphane (0.18 g, 0.42 mmol, 0.2 equiv) in dimethyl sulfoxide (10 mL) was degassed with nitrogen atmosphere. Then tert-butyl isocyanide (0.21 g, 2.5 mmol, 1.2 equiv) was added under nitrogen atmosphere. The resulting suspension was stirred at 120° C. for 12 h under nitrogen atmosphere and cooled to room temperature. Water was added and the resulting precipitate was filtered, washed with water, and dried in an oven. The obtained solid was purified by silica gel column chromatography using a 0-40% gradient of ethyl acetate in dichloromethane to afford tert-butyl N-({4-[2-(2-formamidopyridin-3-yl)-5-formylimidazo[4,5-b]pyridin-3-yl]phenyl}methyl)carbamate (520 mg, 50%) as a yellow solid. MS (ESI) calculated for C₂₅H₂₄N₆O₄: 472.19 m/z, found 472.50 [M+H]⁺.

### Step 3: Synthesis of tert-butyl (4-(5-(difluoromethyl)-2-(2-formamidopyridin-3-yl)-3H-imidazo[4,5-b]pyridin-3-yl)benzyl)carbamate

A solution of tert-butyl N-({4-[2-(2-formamidopyridin-3-yl)-5-formylimidazo[4,5-b]pyridin-3-yl]phenyl}methyl)carbamate (500 mg, 1.06 mmol, 1 equiv) in dichloromethane (15 mL) was treated with a solution of diethylaminosulfur trifluoride (512 mg, 3.17 mmol, 3 equiv) in dichloromethane (10 mL) at 0° C. under nitrogen atmosphere. The mixture was stirred at room temperature overnight. The resulting mixture was diluted with dichloromethane and washed with brine (2×20 mL). The combined organic layers were dried over anhydrous sodium sulfate, filtered, and concentrate in vacuo to provide tert-butyl N-({4-[5-(difluoromethyl)-2-(2-formamidopyridin-3-yl)imidazo[4,5-b]pyridin-3-yl]phenyl}methyl)carbamate (380 mg, 73%) as a yellow solid, which was used without purification in the next step. MS (ESI) calculated for C₂₅H₂₄F₂N₆O₃: 494.19 m/z, found 495.20 [M+H]⁺.

### Step 4: Synthesis of tert-butyl (4-(2-(2-aminopyridin-3-yl)-5-(difluoromethyl)-3H-imidazo[4,5-b]pyridin-3-yl)benzyl)carbamate

A solution of lithium hydroxide (44 mg, 1.8 mmol, 2.5 equiv) in water (4 mL) was added to a solution of tert-butyl N-({4-[5-(difluoromethyl)-2-(2-formamidopyridin-3-yl)imidazo[4,5-b]pyridin-3-yl]phenyl}methyl) carbamate (360 mg, 0.728 mmol, 1 equiv) in tetrahydrofuran (12 mL) and the resulting solution was stirred at room temperature overnight. The solution was then extracted with dichloromethane (3×20 mL). The combined organic layers were washed with brine (50 mL×2), dried with sodium sulfate, filtered, and concentrated in vacuo to afford tert-butyl N-({4-[2-(2-aminopyridin-3-yl)-5-(difluoromethyl)imidazo[4,5-b]pyridin-3-yl]phenyl}methyl)carbamate (334 mg, 98%) as a yellow solid. MS (ESI) calculated for C₂₄H₂₄F₂N₆O₂: 466.19 m/z, found 467.20 [M+H]⁺.

### Step 5: Synthesis of 3-(3-(4-(aminomethyl)phenyl)-5-(difluoromethyl)-3H-imidazo[4,5-b]pyridin-2-yl)pyridin-2-amine (Intermediate 7-1)

To a solution of tert-butyl N-({4-[2-(2-aminopyridin-3-yl)-5-(difluoromethyl) imidazo[4,5-b]pyridin-3-yl]phenyl}methyl)carbamate (360 mg, 0.772 mmol, 1 equiv) in dichloromethane (5 mL) was added 4N hydrochloric acid in 1,4-dioxane (4 mL) and the resulting suspension was stirred at room temperature for 1 h. The mixture was concentrated in vacuo to provide 3-{3-[4-(aminomethyl)phenyl]-5-(difluoromethyl)imidazo[4,5-b]pyridin-2-yl}pyridin-2-amine (Intermediate 7-1) (310 mg, crude quant.) as a yellow solid, which was used without purification in subsequent transformations. MS (ESI) calculated for C₁₉H₁₆F₂N₆: 366.14 m/z, found 367.20 [M+H]⁺.

### Example 8: N-({4-[2-(2-aminopyridin-3-yl)-5-{8-oxa-3-azabicyclo[3.2.1]octan-3-yl}imidazo[4,5-b]pyridin-3-yl]phenyl}methyl)-2-(4-formyl-3-hydroxyphenyl)acetamide

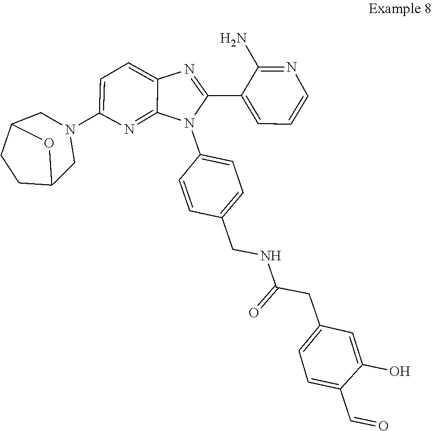 [SMILES: Nc1ncccc1-c1nc2ccc(N3CC4CCC(C3)O4)nc2n1-c1ccc(CNC(=O)Cc2ccc(C=O)c(O)c2)cc1]

Example 8 was prepared in a manner analogous to Example 1 (starting from Step 3) using Intermediate 8-1 in place of the amine starting material. MS (ESI) calculated for C₃₃H₃₁N₇O₄: 589.24 m/z, found 590.15 [M+H]⁺. ¹H NMR (400 MHz, DMSO-d₆) δ (ppm): 10.20 (s, 1H), 7.92-7.95 (m, 2H), 7.61-7.63 (m, 1H), 7.32-7.59 (m, 4H), 6.90-7.00 (m, 2H), 6.82-6.89 (m, 1H), 6.77-6.83 (m, 1H), 6.33-6.35 (m, 1H), 4.37-4.41 (m, 4H), 3.75-3.78 (m, 2H), 3.44-3.52 (m, 2H), 2.89-2.92 (m, 2H), 1.73-1.81 (m, 4H).

### Intermediate 8-1: 3-{3-[4-(aminomethyl)phenyl]-5-{8-oxa-3-azabicyclo[3.2.1]octan-3-yl}imidazo[4,5-b]pyridin-2-yl}pyridin-2-amine

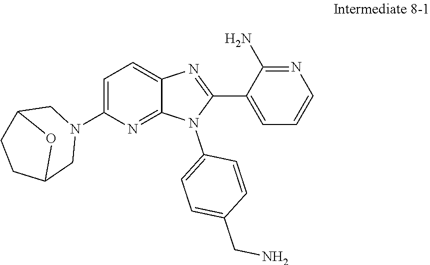 [SMILES: NCc1ccc(-n2c(-c3cccnc3N)nc3ccc(N4CC5CCC(C4)O5)nc32)cc1]

Intermediate 8-1 was prepared in a manner analogous to Intermediate 3-1 using 8-oxa-3-azabicyclo[3.2.1]octane in place of morpholine. MS (ESI) calculated for C₂₄H₂₅N₇O: 427.21 m/z, found 428.21 [M+H]⁺.

### Example 9: N-(4-(2-(2-aminopyridin-3-yl)-5-(3-oxa-8-azabicyclo[3.2.1]octan-8-yl)-3H-imidazo[4,5-b]pyridin-3-yl)benzyl)-2-(4-formyl-3-hydroxyphenyl)acetamide

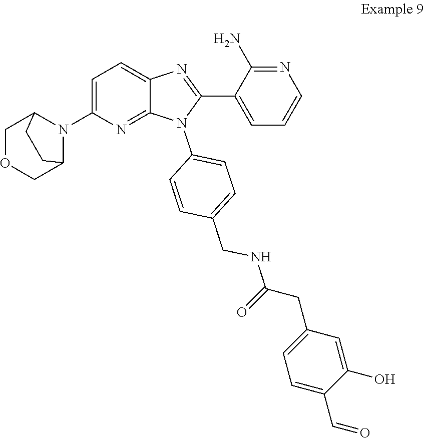 [SMILES: Nc1ncccc1-c1nc2ccc(N3C4CCC3COC4)nc2n1-c1ccc(CNC(=O)Cc2ccc(C=O)c(O)c2)cc1]

Example 9 was prepared in a manner analogous to Example 1 (starting from Step 3) using Intermediate 9-1 in place of the amine starting material. MS (ESI) calculated for C₃₃H₃₁N₇O₄: 589.24 m/z, found 590.25 [M+H]⁺. ¹H NMR (300 MHz, DMSO-d₆) δ (ppm): 10.20 (s, 1H), 8.70-8.74 (m, 1H), 7.92-7.97 (m, 2H), 7.59-7.62 (m, 1H), 7.29-7.35 (m, 4H), 7.02-7.05 (m, 1H), 6.85-6.95 (m, 3H), 6.33-6.37 (m, 1H), 4.30-4.37 (m, 4H), 3.64-3.67 (m, 2H), 3.52-3.54 (m, 3H), 3.45-3.48 (m, 1H), 1.81-1.93 (m, 4H).

### Intermediate 9-1: 3-(3-(4-(aminomethyl)phenyl)-5-(3-oxa-8-azabicyclo[3.2.1]octan-8-yl)-3H-imidazo[4,5-b]pyridin-2-yl)pyridin-2-amine

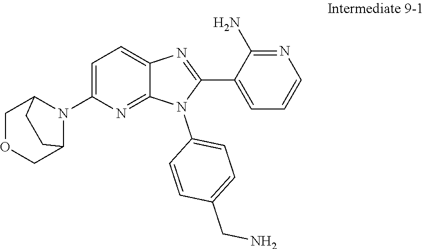 [SMILES: NCc1ccc(-n2c(-c3cccnc3N)nc3ccc(N4C5CCC4COC5)nc32)cc1]

Intermediate 9-1 was prepared in a manner analogous to Intermediate 3-1 using 3-oxa-8-azabicyclo[3.2.1]octane in place of morpholine. MS (ESI) calculated for C₂₄H₂₅N₇O: 427.21 m/z, found 428.25 [M+H]⁺.

### Example 10: N-({4-[2-(2-aminopyridin-3-yl)-5-cyclopropoxyimidazo[4,5-b]pyridin-3-yl]phenyl}methyl)-2-(4-formyl-3-hydroxyphenyl)acetamide

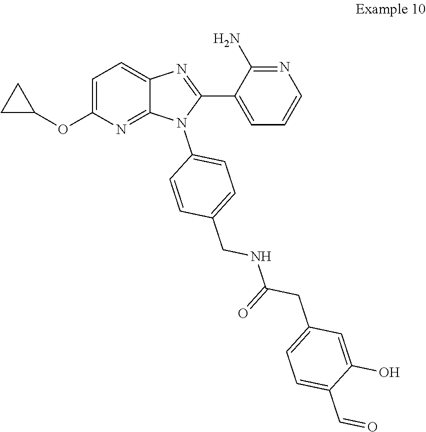 [SMILES: Nc1ncccc1-c1nc2ccc(OC3CC3)nc2n1-c1ccc(CNC(=O)Cc2ccc(C=O)c(O)c2)cc1]

Example 10 was prepared in a manner analogous to Example 1 (starting from Step 3) using Intermediate 10-1 in place of the amine starting material. MS (ESI) calculated for C₃₀H₂₆N₆O₄: 534.20 m/z, found 535.20 [M+H]⁺. ¹H NMR (400 MHz, DMSO-d₆) δ 10.18 (s, 1H), 8.16-8.22 (m, 1H), 7.99-8.05 (m, 1H), 7.56-7.67 (m, 2H), 7.33-7.45 (m, 4H), 6.87-7.00 (m, 3H), 6.71-6.77 (m, 1H), 4.36 (s, 2H), 4.08-4.16 (m, 2H), 0.69-0.76 (m, 2H), 0.63-0.69 (m, 2H).

### Intermediate 10-1: 3-(3-(4-(aminomethyl)phenyl)-5-cyclopropoxy-3H-imidazo[4,5-b]pyridin-2-yl)pyridin-2-amine

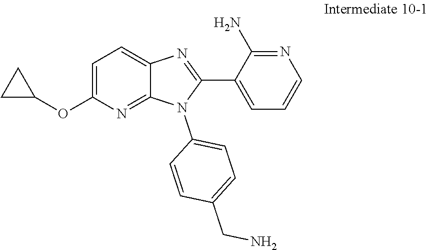 [SMILES: NCc1ccc(-n2c(-c3cccnc3N)nc3ccc(OC4CC4)nc32)cc1]

### Synthetic Route:

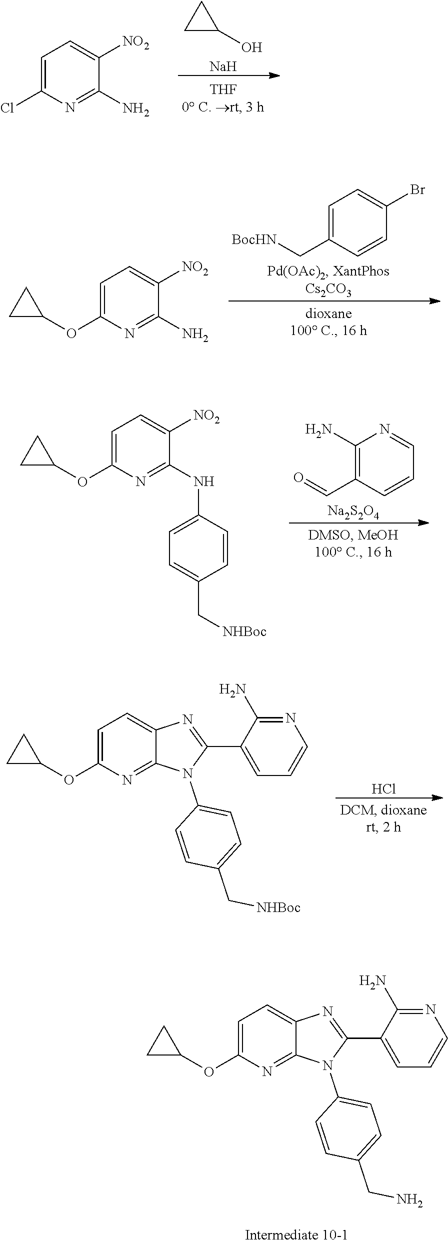

### Step 1: Synthesis of 6-cyclopropoxy-3-nitropyridin-2-amine

A suspension of cyclopropanol (1.34 g, 23.0 mmol, 2 equiv) and sodium hydride (0.55 g, 23 mmol, 2 equiv) in tetrahydrofuran (10 mL) was stirred at 0° C. for 0.5 h under nitrogen atmosphere. Then 6-chloro-3-nitropyridin-2-amine (2.00 g, 11.5 mmol, 1 equiv) was added and the mixture was stirred at room temperature for 3 h. The resulting mixture was concentrated in vacuo and purified by silica gel column chromatography using a 0-50% gradient of ethyl acetate in petroleum ether to afford 6-cyclopropoxy-3-nitropyridin-2-amine (1.5 g, 49%) as a yellow solid. MS (ESI) calculated for C₈H₉N₃O₃: 195.06 m/z, found 196.00 [M+H]⁺.

### Step 2: Synthesis of tert-butyl N-({4-[(6-cyclopropoxy-3-nitropyridin-2-yl)amino]phenyl}methyl)carbamate

A suspension of 6-cyclopropoxy-3-nitropyridin-2-amine (1.5 g, 7.7 mmol, 1 equiv), tert-butyl N-[(4-bromophenyl)methyl]carbamate (3.30 g, 11.5 mmol, 1.5 equiv), palladium(II) acetate (0.17 g, 0.77 mmol, 0.1 equiv), XantPhos (0.89 g, 1.54 mmol, 0.2 equiv) and cesium carbonate (7.51 g, 23.1 mmol, 3 equiv) in 1,4-dioxane (15 mL) was stirred at 100° C. for 16 h under nitrogen atmosphere. The mixture was concentrated in vacuo and the resulting residue was purified by reverse-phase column chromatography on C18 silica gel using a 5-60% gradient of acetonitrile in water (+0.05% 2,2,2-trifluoroacetic acid) to afford tert-butyl N-({4-[(6-cyclopropoxy-3-nitropyridin-2-yl)amino]phenyl}methyl)carbamate (1.5 g, 38%) as a brown/yellow solid. MS (ESI) calculated for C₂₀H₂₄N₄O₅: 400.17 m/z, found 401.10 [M+H]⁺.

### Step 3: Synthesis of tert-butyl N-({4-[2-(2-aminopyridin-3-yl)-5-cyclopropoxyimidazo[4,5-b]pyridin-3-yl]phenyl}methyl)carbamate

A suspension of tert-butyl N-({4-[(6-cyclopropoxy-3-nitropyridin-2-yl)amino]phenyl}methyl)carbamate (1.00 g, 2.50 mmol, 1 equiv), 2-aminopyridine-3-carbaldehyde (0.40 g, 3.2 mmol, 1.3 equiv) and sodium dithionite (0.87 g, 5.0 mmol, 2 equiv) in dimethyl sulfoxide (13 mL) and methanol (2 mL) was stirred at 100° C. for 16 h. The mixture was cooled to room temperature and purified by reverse-phase column chromatography on C18 silica gel using a 5-70% gradient of acetonitrile in water (+0.05% 2,2,2-trifluoroacetic acid) to afford tert-butyl N-({4-[2-(2-aminopyridin-3-yl)-5-cyclopropoxyimidazo[4,5-b]pyridin-3-yl]phenyl}methyl)carbamate (360 mg, 18%) as a yellow solid. MS (ESI) calculated for C₂₆H₂₈N₆O₃: 472.22 m/z, found 473.35 [M+H]⁺.

### Step 4: Synthesis of 3-(3-(4-(aminomethyl)phenyl)-5-cyclopropoxy-3H-imidazo[4,5-b]pyridin-2-yl)pyridin-2-amine (Intermediate 10-1)

A suspension of tert-butyl N-({4-[2-(2-aminopyridin-3-yl)-5-cyclopropoxy imidazo[4,5-b]pyridin-3-yl]phenyl}methyl)carbamate (360 mg, 0.76 mmol, 1 equiv) in 4N hydrochloric acid in 1,4-dioxane (1 mL) and dichloromethane (5 mL) was stirred at room temperature for 2 h. The mixture was concentrated in vacuo to provide 3-{3-[4-(aminomethyl)phenyl]-5-cyclopropoxyimidazo[4,5-b]pyridin-2-yl}pyridin-2-amine (Intermediate 10-1) (360 mg, 57%) as a yellow solid, which was used without further purification in subsequent transformations. MS (ESI) calculated for C₂₁H₂₀N₆O: 372.17 m/z, found 373.05 [M+H]⁺.

### Example 11: 2-{4-[2-(2-aminopyridin-3-yl)-5-phenylimidazo[4,5-b]pyridin-3-yl]phenyl}-N-[1-(4-formyl-3-hydroxyphenyl)cyclopropyl]acetamide

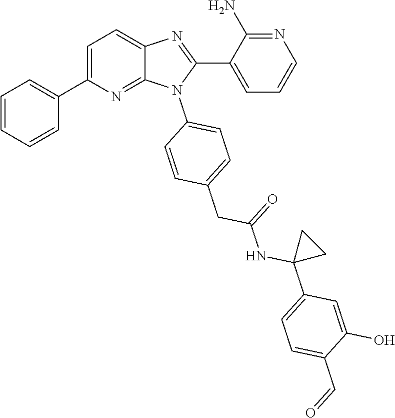 [SMILES: Nc1ncccc1-c1nc2ccc(-c3ccccc3)nc2n1-c1ccc(CC(=O)NC2(c3ccc(C=O)c(O)c3)CC2)cc1]

Example 11 was prepared in a manner analogous to Example 1 (starting from Step 3) using Intermediate 11-1 in place of Intermediate 1-4 and Intermediate 11-2 in place of the amine starting material. MS (ESI) calculated for C₃₅H₂₈N₆O₃: 580.22 m/z, found 581.25 [M+H]⁺. ¹H NMR (400 MHz, DMSO-d₆) δ (ppm): 10.10 (s, 1H), 8.23-8.30 (m, 1H), 7.95-8.09 (m, 4H), 7.38-7.55 (m, 9H), 7.18-7.21 (m, 1H), 6.75 (s, 1H), 6.61-6.69 (m, 1H), 6.35-6.40 (m, 1H), 3.70 (s, 2H), 1.19-1.31 (m, 4H).

### Intermediate 11-1: 2-(4-(2-(2-aminopyridin-3-yl)-5-phenyl-3H-imidazo[4,5-b]pyridin-3-yl)phenyl)acetic acid

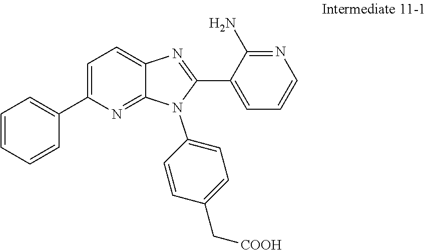 [SMILES: Nc1ncccc1-c1nc2ccc(-c3ccccc3)nc2n1-c1ccc(CC(=O)O)cc1]

### Synthetic Route:

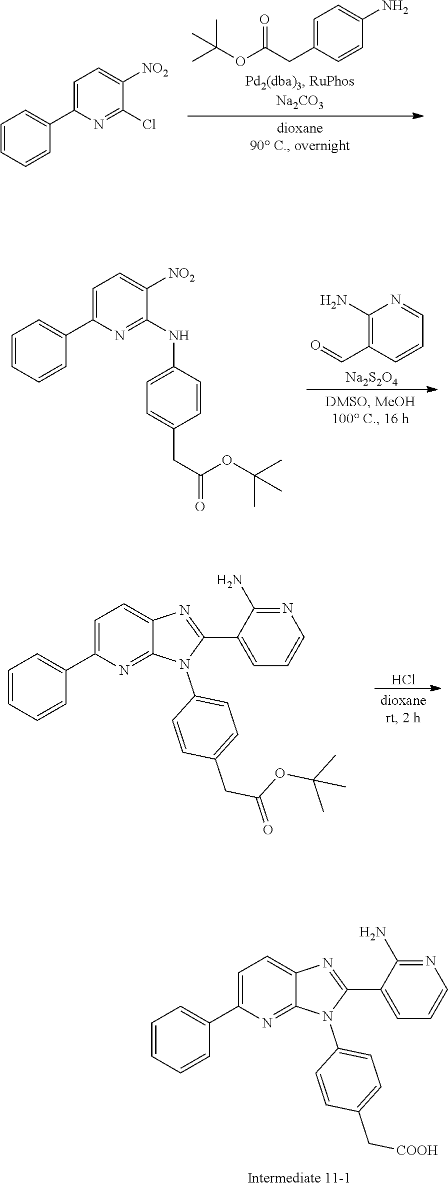

### Step 1: Synthesis of tert-butyl 2-{4-[(3-nitro-6-phenylpyridin-2-yl)amino]phenyl}acetate

A mixture of 2-chloro-3-nitro-6-phenylpyridine (1.08 g, 4.60 mmol, 1 equiv), tert-butyl 2-(4-aminophenyl)acetate (1.05 g, 5.06 mmol, 1.1 equiv), tris(dibenzylideneacetone) dipalladium(0) (0.42 g, 0.46 mmol, 0.1 equiv), RuPhos (0.21 g, 0.46 mmol, 0.1 equiv) and sodium carbonate (0.98 g, 9.2 mmol, 2 equiv) in 1,4-dioxane (50 mL) was stirred overnight at 90° C. under nitrogen atmosphere. The mixture was cooled to room temperature and concentrated in vacuo. The resulting residue was purified by silica gel column chromatography using a 0-20% gradient of ethyl acetate in petroleum ether to afford tert-butyl 2-{4-[(3-nitro-6-phenylpyridin-2-yl)amino]phenyl}acetate (1 g, 48%) as brown/yellow solid. MS (ESI) calculated for C₂₃H₂₃N₃O₄: 405.17 m/z, found 406.10 [M+H]⁺.

### Step 2: Synthesis of tert-butyl 2-{4-[2-(2-aminopyridin-3-yl)-5-phenylimidazo[4,5-b]pyridin-3-yl]phenyl}acetate

A mixture of tert-butyl 2-{4-[(3-nitro-6-phenylpyridin-2-yl)amino]phenyl}acetate (1.00 g, 2.47 mmol, 1 equiv), 2-aminopyridine-3-carbaldehyde (361 mg, 2.96 mmol, 1.2 equiv) and sodium dithionite (1.07 g, 6.17 mmol, 2.5 equiv) in dimethyl sulfoxide (24 mL) and methanol (2 mL) was stirred overnight at 100° C. The mixture was cooled to room temperature, partially concentrated in vacuo and purified by reverse-flash column chromatography on C18 silica gel using a 10-70% gradient of acetonitrile in water (+0.05% ammonium bicarbonate) to provide tert-butyl 2-{4-[2-(2-aminopyridin-3-yl)-5-phenylimidazo[4,5-b]pyridin-3-yl]phenyl}acetate (700 mg, 51%) as a reddish brown solid. MS (ESI) calculated for C₂₉H₂₇N₅O₂: 477.22 m/z, found 478.10 [M+H]⁺.

### Step 3: Synthesis of {4-[2-(2-aminopyridin-3-yl)-5-phenylimidazo[4,5-b]pyridin-3-yl]phenyl}acetic acid (Intermediate 11-1)

A mixture of tert-butyl 2-{4-[2-(2-aminopyridin-3-yl)-5-phenylimidazo[4,5-b]pyridin-3-yl]phenyl}acetate (400 mg, 0.838 mmol, 1 equiv) in 4N hydrochloric acid in 1,4-dioxane (4.2 mL) was stirred for 1 h at room temperature. The resulting mixture was concentrated in vacuo to afford {4-[2-(2-aminopyridin-3-yl)-5-phenylimidazo[4,5-b]pyridin-3-yl]phenyl}acetic acid (Intermediate 11-1) (220 mg, 53%) as a yellow solid, which was used without further purification in subsequent transformations. MS (ESI) calculated for C₂₅H₁₉N₅O₂: 421.15 m/z, found 422.05 [M+H]⁺.

### Intermediate 11-2: 1-(4-(1,3-dioxolan-2-yl)-3-((4-methoxybenzyl)oxy)phenyl)cyclopropan-1-amine

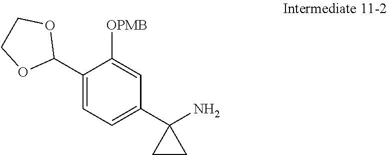

### Synthetic Route:

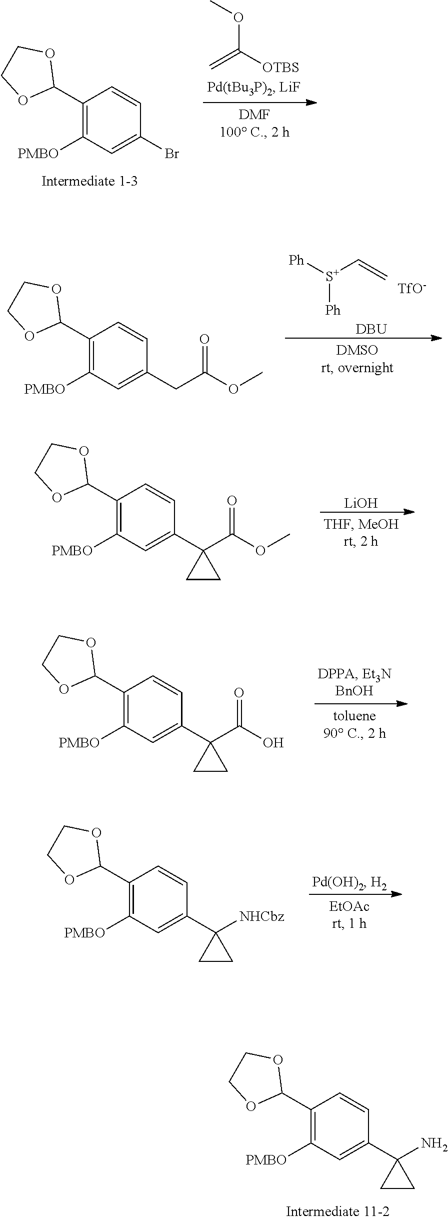

### Step 1: Synthesis of methyl 2-(4-(1,3-dioxolan-2-yl)-3-((4-methoxybenzyl)oxy)phenyl) acetate

To a solution of 2-(4-bromo-2-((4-methoxybenzyl)oxy)phenyl)-1,3-dioxolane (Intermediate 1-3) (4.15 g, 11.3 mmol, 1 equiv) and tert-butyldimethylsilyl methyl carbonate (8.65 g, 45.4 mmol, 4 equiv) in N,N-dimethylformamide (20 mL) were added bis(tri-tert-butylphosphine)palladium(0) (0.60 g, 1.14 mmol, 0.1 equiv) and lithium fluoride (0.6 g, 23 mmol, 2 equiv). The resulting mixture was stirred at 100° C. under nitrogen atmosphere for 2 h then cooled to 0° C. and quenched with water (100 mL). The resulting mixture was extracted with ethyl acetate (200 mL×3). The combined organic layers were washed with water (100 mL×3), dried over anhydrous sodium sulfate, filtered, and concentrated in vacuo. The residue was purified by silica gel column chromatography using a 0-60% gradient of ethyl acetate in petroleum ether to provide methyl 2-(4-(1,3-dioxolan-2-yl)-3-((4-methoxybenzyl)oxy) phenyl)acetate (3.0 g, 74%) as a light-yellow oil. MS (ESI) calculated for C₂₀H₂₂O₆: 358.14 m/z, found 359.10 [M+H]⁺.

### Step 2: Synthesis of methyl 1-(4-(1,3-dioxolan-2-yl)-3-((4-methoxybenzyl)oxy)phenyl) cyclopropane-1-carboxylate

A mixture of methyl 2-(4-(1,3-dioxolan-2-yl)-3-((4-methoxybenzyl)oxy)phenyl)acetate (1.0 g, 2.80 mmol, 1 equiv), ethenyldiphenylsulfanium triflate (0.89 g, 4.2 mmol, 1.5 equiv) and 1,8-diazabicyclo[5.4.0]undec-7-ene (1.27 g, 8.36 mmol, 3.0 equiv) in dimethyl sulfoxide (15 mL) was stirred at room temperature overnight. The reaction mixture was quenched by addition of water (50 mL) and extracted with ethyl acetate (50 mL). The combined organic layers were dried over sodium sulfate, filtered, and concentrated in vacuo. The resulting residue was purified by silica gel column chromatography using a 0-100% gradient of ethyl acetate in petroleum ether to afford methyl 1-(4-(1,3-dioxolan-2-yl)-3-((4-methoxybenzyl)oxy)phenyl)cyclopropane-1-carboxylate (800 mg, 75%) as a yellow oil. MS (ESI) calculated for C₂₂H₂₄O₆: 384.16 m/z, found 385.10 [M+H]⁺.

### Step 3: Synthesis of 1-(4-(1,3-dioxolan-2-yl)-3-((4-methoxybenzyl)oxy)phenyl)cyclopropane-1-carboxylic acid

To a solution of methyl 1-(4-(1,3-dioxolan-2-yl)-3-((4-methoxybenzyl)oxy)phenyl) cyclopropane-1-carboxylate (800 mg, 2.08 mmol, 1 equiv) in tetrahydrofuran (10 mL) and water (10 mL) was added 2M aqueous lithium hydroxide (3.0 mL, 3 equiv). The resulting mixture was stirred at room temperature for 2 h. The mixture was concentrated in vacuo and purified by reverse-phase flash column chromatography on C18 silica gel using a 20-95% gradient of acetonitrile in water (+0.05% ammonium bicarbonate) with a 10-minute hold at 70% acetonitrile to afford 1-(4-(1,3-dioxolan-2-yl)-3-((4-methoxybenzyl)oxy)phenyl)cyclopropane-1-carboxylic acid (600 mg, 78%) as a white solid. MS (ESI) calculated for C₂₁H₂₂O₆: 370.14 m/z, found 371.10 [M+H]⁺.

### Step 4: Synthesis of benzyl N-{1-[4-(1,3-dioxolan-2-yl)-3-[(4-methoxyphenyl)methoxy]phenyl]cyclopropyl}carbamate

A solution of 1-(4-(1,3-dioxolan-2-yl)-3-((4-methoxybenzyl)oxy)phenyl)cyclopropane-1-carboxylic acid (600 mg, 1.62 mmol, 1 equiv), diphenylphosphoryl azide (665 mg, 2.42 mmol, 1.5 equiv), triethylamine (0.49 g, 4.8 mmol, 3.0 equiv) and benzyl alcohol (876 mg, 8.10 mmol, 5.0 equiv) in toluene (20 mL) was stirred for 2 h at 90° C. under nitrogen atmosphere. The mixture was cooled to room temperature and concentrated in vacuo. The resulting residue was purified by silica gel column chromatography using a 0-50% gradient of ethyl acetate in petroleum ether to afford benzyl N-{1-[4-(1,3-dioxolan-2-yl)-3-[(4-methoxyphenyl)methoxy]phenyl]cyclopropyl}carbamate (400 mg, 26%) as a yellow oil. MS (ESI) calculated for C₂₈H₂₉NO₆: 475.20 m/z, found 476.10 [M+H]⁺.

### Step 5: Synthesis of 1-(4-(1,3-dioxolan-2-yl)-3-((4-methoxybenzyl)oxy)phenyl)cyclopropan-1-amine (Intermediate 11-2)

To a solution of benzyl N-{1-[4-(1,3-dioxolan-2-yl)-3-[(4-methoxyphenyl) methoxy]phenyl]cyclopropyl}carbamate (400 mg, 0.841 mmol, 1 equiv) in ethyl acetate (30 mL) was added 20% palladium(II) hydroxide on carbon (40 mg, 0.056 mmol, 0.06 equiv). The reaction was stirred at room temperature for 2 h under an atmosphere of H₂. The resulting mixture was filtered, concentrated in vacuo, and purified by silica gel chromatography using a 0-100% gradient of ethyl acetate in petroleum ether to afford 1-(4-(1,3-dioxolan-2-yl)-3-((4-methoxybenzyl)oxy)phenyl)cyclopropan-1-amine (Intermediate 11-2) (160 mg, 56%) as a yellow solid. MS (ESI) calculated for C₂₀H₂₃NO₄: 341.16 m/z, found 342.10 [M+H]⁺.

### Example 12: N-(4-(2-(2-aminopyridin-3-yl)-5-(3-morpholinophenyl)-3H-imidazo[4,5-b]pyridin-3-yl)benzyl)-3-fluoro-5-formyl-4-hydroxybenzamide

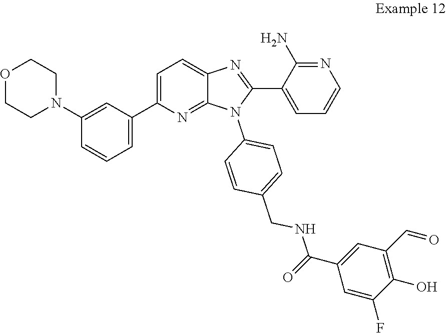 [SMILES: Nc1ncccc1-c1nc2ccc(-c3cccc(N4CCOCC4)c3)nc2n1-c1ccc(CNC(=O)c2cc(F)c(O)c(C=O)c2)cc1]

### Synthetic Route:

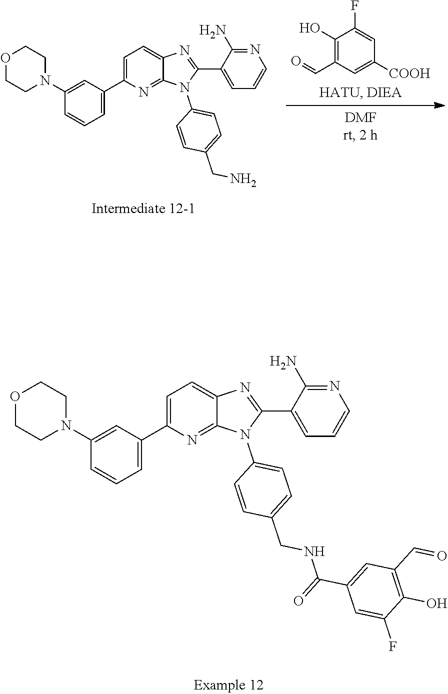

### Step 1: Synthesis of N-(4-(2-(2-aminopyridin-3-yl)-5-(3-morpholinophenyl)-3H-imidazo[4,5-b]pyridin-3-yl)benzyl)-3-fluoro-5-formyl-4-hydroxybenzamide (Example 12)

A mixture of 3-{3-[4-(aminomethyl)phenyl]-5-[3-(morpholin-4-yl)phenyl]imidazo[4,5-b]pyridin-2-yl}pyridin-2-amine (Intermediate 12-1) (200 mg, 0.42 mmol, 1 equiv) and N,N-diisopropylethylamine (217 mg, 1.68 mmol, 4 equiv) in N,N-dimethylformamide (2 mL) was stirred for 5 min at room temperature. To this mixture was added 3-fluoro-5-formyl-4-hydroxybenzoic acid (77 mg, 0.42 mmol, 1 equiv) and HATU (159 mg, 0.42 mmol, 1 equiv). The resulting mixture was stirred for 2 h at room temperature. The mixture was purified by reverse-phase flash chromatography on C18 silica gel using a 10-50% gradient of acetonitrile in water to afford N-({4-[2-(2-aminopyridin-3-yl)-5-[3-(morpholin-4-yl)phenyl]imidazo[4,5-b]pyridin-3-yl]phenyl}methyl)-3-fluoro-5-formyl-4-hydroxybenzamide (Example 12) (7.9 mg, 3%) as a yellow solid. MS (ESI) calculated for C₃₆H₃₀FN₇O₄: 643.23 m/z, found 644.35 [M+H]⁺. ¹H NMR (400 MHz, DMSO-d₆) δ 10.32 (s, 1H), 8.31-8.40 (m, 1H), 8.13-8.20 (m, 1H), 8.01-8.10 (m, 3H), 7.78-7.88 (m, 1H), 7.59-7.65 (m, 1H), 7.46-7.60 (m, 5H), 7.30-7.41 (m, 1H), 6.95-7.09 (m, 1H), 7.78-7.89 (m, 1H), 4.50-4.60 (m, 2H), 3.71-3.80 (m, 4H), 3.12-3.21 (m, 4H).

### Intermediate 12-1: 3-(3-(4-(aminomethyl)phenyl)-5-(3-morpholinophenyl)-3H-imidazo[4,5-b]pyridin-2-yl)pyridin-2-amine

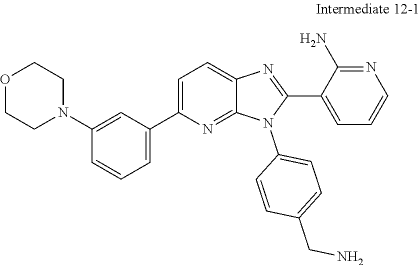 [SMILES: NCc1ccc(-n2c(-c3cccnc3N)nc3ccc(-c4cccc(N5CCOCC5)c4)nc32)cc1]

### Synthetic Route:

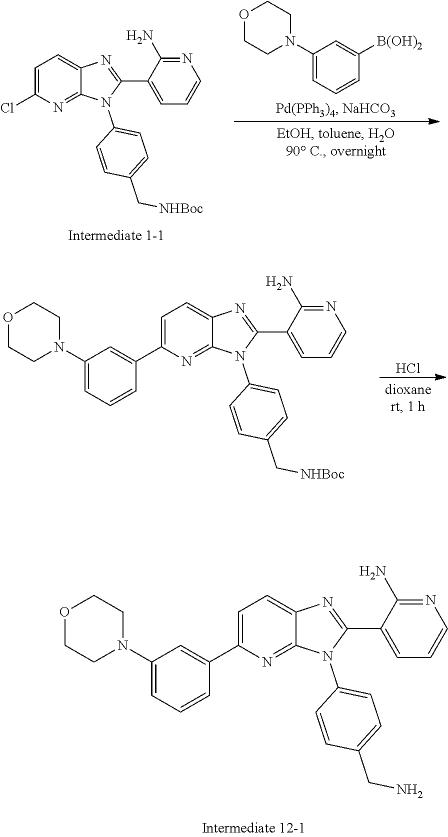 [SMILES: CC(C)(C)OC(=O)NCc1ccc(-n2c(-c3cccnc3N)nc3ccc(Cl)nc32)cc1 ; OB(O)c1cccc(N2CCOCC2)c1 ; CC(C)(C)OC(=O)NCc1ccc(-n2c(-c3cccnc3N)nc3ccc(-c4cccc(N5CCOCC5)c4)nc32)cc1 ; Cl ; NCc1ccc(-n2c(-c3cccnc3N)nc3ccc(-c4cccc(N5CCOCC5)c4)nc32)cc1]

### Step 1: Synthesis of tert-butyl (4-(2-(2-aminopyridin-3-yl)-5-(3-morpholinophenyl)-3H-imidazo[4,5-b]pyridin-3-yl)benzyl)carbamate

To a solution of tert-butyl N-({4-[2-(2-aminopyridin-3-yl)-5-chloroimidazo[4,5-b]pyridin-3-yl]phenyl}methyl)carbamate (Intermediate 1-1) (200 mg, 0.44 mmol, 1 equiv) and (3-morpholinophenyl)boronic acid (368 mg, 1.78 mmol, 4 equiv) in ethanol (5 mL), toluene (5 mL) and water (1 mL) were added sodium bicarbonate (149 mg, 1.78 mmol, 4 equiv) and tetrakis(triphenylphosphine)palladium(0) (51 mg, 0.044 mmol, 0.1 equiv). After stirring overnight at 90° C. under a nitrogen atmosphere, the mixture was concentrated under reduced pressure and purified by reverse-phase flash column chromatography on C18 silica gel using a 10-50% gradient of acetonitrile in water to afford tert-butyl (4-(2-(2-aminopyridin-3-yl)-5-(3-morpholinophenyl)-3H-imidazo[4,5-b]pyridin-3-yl)benzyl)carbamate (160 mg, 62%) as a yellow solid. MS (ESI) calculated for C₃₃H₃₅N₇O₃: 577.28 m/z, found 578.10 [M+H]⁺.

### Step 2: Synthesis of 3-(3-(4-(aminomethyl)phenyl)-5-(3-morpholinophenyl)-3H-imidazo[4,5-b]pyridin-2-yl)pyridin-2-amine (Intermediate 12-1)

To a solution of tert-butyl (4-(2-(2-aminopyridin-3-yl)-5-(3-morpholinophenyl)-3H-imidazo[4,5-b]pyridin-3-yl)benzyl)carbamate (151 mg, 0.262 mmol, 1 equiv) in 1,4-dioxane (5 mL) was added 4N hydrochloric acid in 1,4-dioxane (1 mL) and the resulting mixture was stirred at room temperature for 1 h. The resulting mixture was concentrated in vacuo to afford 3-(3-(4-(aminomethyl)phenyl)-5-(3-morpholinophenyl)-3H-imidazo[4,5-b]pyridin-2-yl)pyridin-2-amine (Intermediate 12-1) (110 mg, 88%) as a yellow solid, which was used without further purification in subsequent transformations. MS (ESI) calculated for C₂₈H₂₇N₇O: 477.23 m/z, found 478.10 [M+H]⁺.

### Example 13: N-({4-[2-(2-aminopyridin-3-yl)-5-[3-(morpholin-4-yl)phenyl]imidazo[4,5-b]pyridin-3-yl]phenyl}methyl)-2-(2-chloro-4-formyl-3-hydroxyphenyl)acetamide

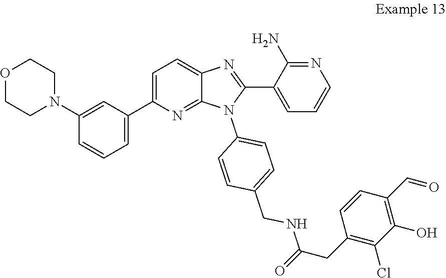 [SMILES: Nc1ncccc1-c1nc2ccc(-c3cccc(N4CCOCC4)c3)nc2n1-c1ccc(CNC(=O)Cc2ccc(C=O)c(O)c2Cl)cc1]

Example 13 was prepared in a manner analogous to Example 1 (starting from Step 3) using Intermediate 12-1 in place of the amine starting material and Intermediate 13-2 in place of Intermediate 1-4. MS (ESI) calculated for C₃₇H₃₂ClN₇O₄: 673.22 m/z, found 674.30 [M+H]⁺. ¹H NMR (400 MHz, DMSO-d₆) δ (ppm): 10.22 (s, 1H), 8.15-8.35 (m, 1H), 7.90-8.10 (m, 2H), 7.60-7.75 (m, 2H), 7.40-7.60 (m, 5H), 7.20-7.40 (m, 2H), 6.90-7.10 (m, 2H), 6.30-6.50 (m, 1H), 4.25-4.55 (m, 2H), 3.74-3.80 (m, 4H), 3.40-3.44 (m, 2H), 3.05-3.25 (m, 4H).

### Intermediate 13-2: 2-(2-chloro-4-(1,3-dioxolan-2-yl)-3-((4-methoxybenzyl)oxy)phenyl) acetic acid

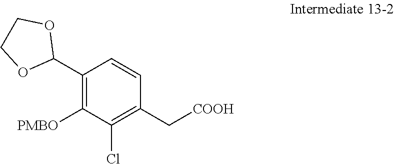

Intermediate 13-2 was prepared in a manner analogous to Intermediate 1-4 using Intermediate 13-1 in place of Intermediate 1-3. MS (ESI) calculated for C₁₉H₁₉ClO₆: 378.09 m/z, found 333.09 [M+H-CO₂]⁺.

### Intermediate 13-1: 2-(4-bromo-3-chloro-2-((4-methoxybenzyl)oxy)phenyl)-1,3-dioxolane

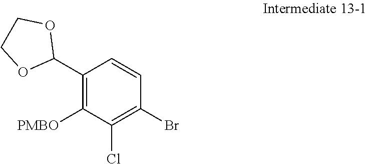

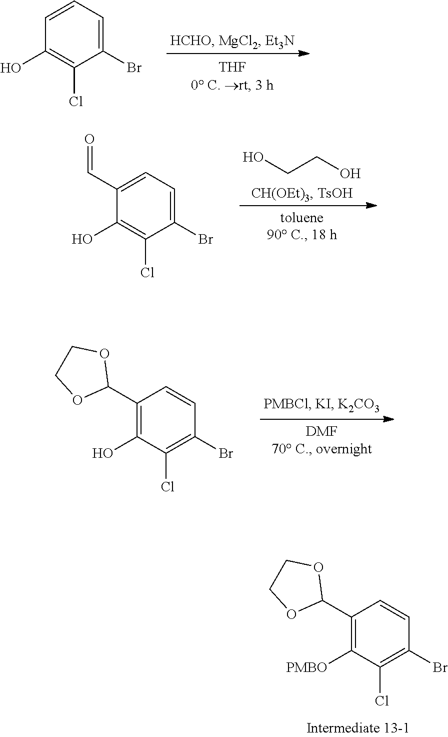

### Step 1: Synthesis of 4-bromo-3-chloro-2-hydroxybenzaldehyde

To a solution of 3-bromo-2-chlorophenol (5 g, 24.102 mmol, 1 equiv) in tetrahydrofuran (50 mL) were added paraformaldehyde (10.86 g, 120.5 mmol, 5 equiv), magnesium(II) chloride (3.44 g, 36.2 mmol, 1.5 equiv) and triethylamine (6.10 g, 60.3 mmol, 2.5 equiv). The resulting suspension was stirred for 2 h at 80° C. The mixture was cooled to room temperature and filtered, rinsing with methanol (50 mL×3). The filtrate was concentrated under reduced pressure and the residue was recrystallized from methanol (10 mL) and ethyl acetate (200 mL) to provide 4-bromo-3-chloro-2-hydroxybenzaldehyde (3 g, 59%) as a light-yellow solid. MS (ESI) calculated for C₇H₄BrClO₂: 233.91 m/z, found 232.95 [M−H]⁻.

### Step 2: Synthesis of 3-bromo-2-chloro-6-(1,3-dioxolan-2-yl)phenol

To a solution of 4-bromo-3-chloro-2-hydroxybenzaldehyde (3.00 g, 12.7 mmol, 1 equiv) in toluene (30 mL) was added ethylene glycol (5.56 g, 89.2 mmol, 7 equiv), triethyl orthoformate (5.67 g, 38.2 mmol, 3 equiv) and p-toluenesulfonic acid (0.12 g, 0.64 mmol, 0.05 equiv). The resulting solution was stirred at room temperature for 10 min then at 90° C. overnight. The mixture was concentrated and purified by silica gel column chromatography using a 0-30% gradient of ethyl acetate in petroleum ether to provide 3-bromo-2-chloro-6-(1,3-dioxolan-2-yl)phenol (2 g, 55%) as a yellow oil. MS (ESI) calculated for C₉H₈BrClO₃: 277.93 m/z, found 279.10 [M+H]⁺.

### Step 3: Synthesis of 2-(4-bromo-3-chloro-2-((4-methoxybenzyl)oxy)phenyl)-1,3-dioxolane (Intermediate 13-1)

A mixture of 3-bromo-2-chloro-6-(1,3-dioxolan-2-yl)phenol (2.00 g, 7.16 mmol, 1 equiv), p-methoxybenzyl chloride (1.34 g, 8.59 mmol, 1.2 equiv), potassium iodide (0.89 g, 0.72 mmol, 0.1 equiv) and potassium carbonate (2.97 g, 21.5 mmol, 3 equiv) in N,N-dimethylformamide (20 mL) was stirred at 70° C. overnight under nitrogen atmosphere. The mixture was cooled to room temperature and diluted with water (40 mL). The mixture was extracted with ethyl acetate (50 mL×3). The combined organic phases were washed with brine (100 mL×3), dried with sodium sulfate, filtered, and concentrated in vacuo. The residue was purified by silica gel column chromatography using a 0-5% gradient of ethyl acetate in petroleum ether to provide 2-(4-bromo-3-chloro-2-((4-methoxybenzyl)oxy)phenyl)-1,3-dioxolane (Intermediate 13-1) (2.4 g, 85%) as a yellow oil. MS (ESI) calculated for C₁₇H₁₆BrClO₄: 397.99 m/z, found 398.95 [M+H]⁺.

### Example 14: N-({4-[2-(2-aminopyridin-3-yl)-5-phenylimidazo[4,5-b]pyridin-3-yl]phenyl}methyl)-2-(4-formyl-3-hydroxy-2-methylphenyl)acetamide

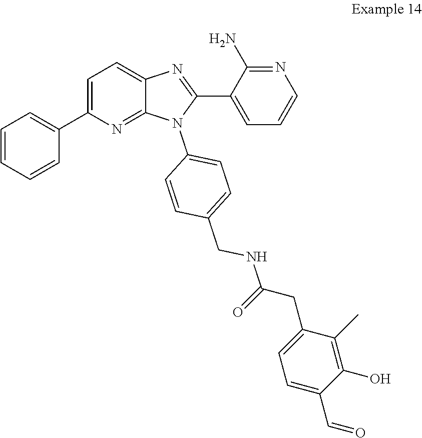 [SMILES: Cc1c(CC(=O)NCc2ccc(-n3c(-c4cccnc4N)nc4ccc(-c5ccccc5)nc43)cc2)ccc(C=O)c1O]

Example 14 was prepared in a manner analogous to Example 1 (starting from Step 3) using Intermediate 4-1 in place of the amine starting material and Intermediate 14-1 in place of Intermediate 1-4. MS (ESI) calculated for C₃₄H₂₈N₆O₃: 568.22 m/z, found 569.20 [M+H]⁺. ¹H NMR (300 MHz, DMSO-d₆) δ ppm: 9.94 (s, 1H), 8.66-8.71 (m, 1H), 8.13-8.32 (m, 1H), 7.90-7.09 (m, 4H), 7.37-7.56 (m, 8H), 7.08-7.23 (m, 1H), 6.88-7.00 (m, 1H), 6.30-6.44 (m, 1H), 4.39-4.31 (m, 2H), 3.67 (s, 2H), 2.52 (s, 3H).

### Intermediate 14-1: 2-(4-formyl-3-hydroxy-2-methylphenyl)acetic acid

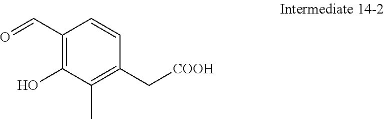

Intermediate 14-1 was prepared in a manner analogous to Intermediate 13-2 (via Intermediate 13-1) starting from 3-bromo-2-methylphenol in place of 3-bromo-2-chlorophenol. MS (ESI) calculated for C₂₀H₂₂O₆: 358.14 m/z, found 359.15 [M+H]⁺.

### Example 15: N-(4-(2-(2-aminopyridin-3-yl)-5-phenyl-3H-imidazo[4,5-b]pyridin-3-yl)benzyl)-2-fluoro-3-formyl-4-hydroxybenzamide

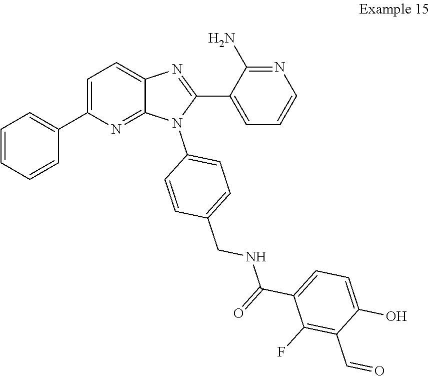 [SMILES: Nc1ncccc1-c1nc2ccc(-c3ccccc3)nc2n1-c1ccc(CNC(=O)c2ccc(O)c(C=O)c2F)cc1]

Example 15 was prepared in a manner analogous to Example 1 (starting from Step 3) using Intermediate 4-1 in place of the amine starting material and Intermediate 15-1 in place of Intermediate 1-4. MS (ESI) calculated for C₃₂H₂₃FN₆O₃: 558.18 m/z, found 559.10 [M+H]⁺. ¹H NMR (400 MHz, DMSO-d₆) δ (ppm): δ 10.30 (s, 1H), 8.29-8.34 (m, 1H), 7.99-8.08 (m, 4H), 7.81-7.90 (m, 1H), 7.60-7.65 (m, 1H), 7.39-7.54 (m, 7H), 6.89-6.95 (m, 1H), 6.67-6.74 (m, 1H), 4.58 (s, 2H). ¹⁹F NMR (400 MHz, DMSO-d₆) δ (ppm): −177.54.

### Intermediate 15-1: 4-(1,3-dioxolan-2-yl)-2-fluoro-3-((4-methoxybenzyl)oxy)benzoic acid

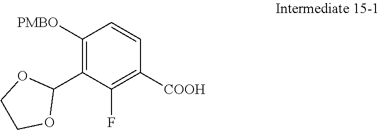

Intermediate 15-1 was prepared in a manner analogous to Intermediate 3-4 (via Intermediates 3-3 and 3-2) starting from 3-bromo-2-fluoro-6-hydroxybenzaldehyde in place of 5-bromo-2-hydroxybenzaldehyde. MS (ESI) calculated for C₁₈H₁₇FO₆: 348.10 m/z, found 349.15 [M+H]⁺.

### Example 16: 4-{2-[({4-[2-(2-aminopyridin-3-yl)-5-cyclopropylimidazo[4,5-b]pyridin-3-yl]phenyl}methyl)amino]ethyl}-2-hydroxybenzaldehyde

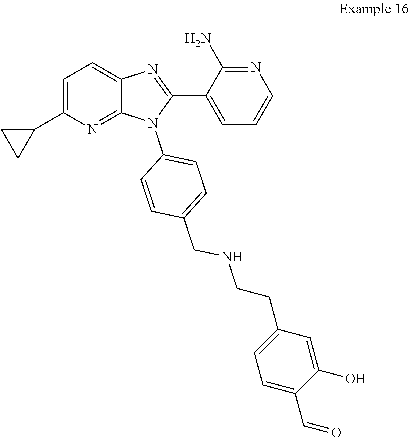 [SMILES: Nc1ncccc1-c1nc2ccc(C3CC3)nc2n1-c1ccc(CNCCc2ccc(C=O)c(O)c2)cc1]

### Synthetic Route:

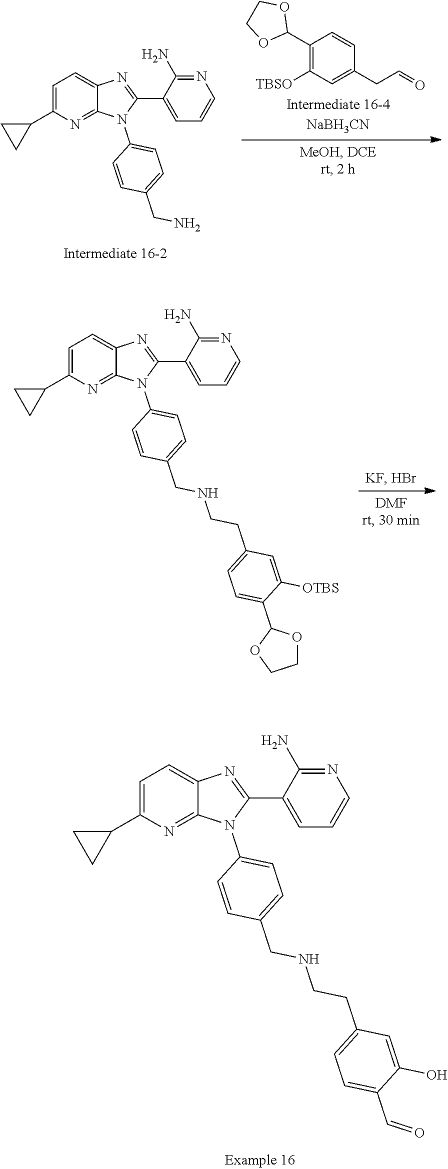 [SMILES: NCc1ccc(-n2c(-c3cccnc3N)nc3ccc(C4CC4)nc32)cc1 ; CC(C)(C)[Si](C)(C)Oc1cc(CC=O)ccc1C1OCCO1 ; CC(C)(C)[Si](C)(C)Oc1cc(CCNCc2ccc(-n3c(-c4cccnc4N)nc4ccc(C5CC5)nc43)cc2)ccc1C1OCCO1 ; Nc1ncccc1-c1nc2ccc(C3CC3)nc2n1-c1ccc(CNCCc2ccc(C=O)c(O)c2)cc1]

### Step 1: Synthesis of 3-[3-(4-{[(2-{3-[(tert-butyldimethylsilyl)oxy]-4-(1,3-dioxolan-2-yl) phenyl}ethyl)amino]methyl}phenyl)-5-cyclopropylimidazo[4,5-b]pyridin-2-yl]pyridin-2-amine

To a stirred solution of 3-{3-[4-(aminomethyl)phenyl]-5-cyclopropylimidazo[4,5-b]pyridin-2-yl}pyridin-2-amine (Intermediate 16-2) (100 mg, 0.281 mmol, 1 equiv) in anhydrous methanol (1.7 mL) and 1,2-dichloroethane (2 mL) was added 2-{3-[(tert-butyldimethylsilyl)oxy]-4-(1,3-dioxolan-2-yl)phenyl}acetaldehyde (Intermediate 16-4) (90 mg, 0.28 mmol, 1 equiv) and the mixture was stirred at room temperature for 0.5 h. Then, sodium cyanoborohydride (35 mg, 0.56 mmol, 2 equiv) was added and the mixture was stirred for 2 h. The mixture was concentrated in vacuo and purified by reverse-phase flash column chromatography on C18 silica gel using a 20-70% gradient of acetonitrile in water (+0.05% 2,2,2-trifluoroacetic acid) to provide 3-[3-(4-{[(2-{3-[(tert-butyldimethylsilyl)oxy]-4-(1,3-dioxolan-2-yl)phenyl}ethyl)amino]methyl}phenyl)-5-cyclopropylimidazo[4,5-b]pyridin-2-yl]pyridin-2-amine (100 mg, 54%) as alight yellow solid. MS (ESI) calculated for C₃₈H₄₆N₆O₃Si: 662.34 m/z, found 663.35 [M+H]⁺.

### Step 2: Synthesis of 4-{2-[({4-[2-(2-aminopyridin-3-yl)-5-cyclopropylimidazo[4,5-b]pyridin-3-yl]phenyl}methyl)amino]ethyl}-2-hydroxybenzaldehyde (Example 16)

To a stirred solution of 3-[3-(4-{[(2-{3-[(tert-butyldimethylsilyl)oxy]-4-(1,3-dioxolan-2-yl)phenyl}ethyl)amino]methyl}phenyl)-5-cyclopropylimidazo[4,5-b]pyridin-2-yl]pyridin-2-amine (100 mg, 0.151 mmol, 1 equiv) in anhydrous N,N-dimethylformamide (1.5 mL) were added potassium fluoride (8.8 mg, 0.15 mmol, 1 equiv) and hydrogen bromide (0.2 mL, 30% in water) and the mixture was stirred at room temperature for 0.5 h. The resulting mixture was diluted with water and extracted with dichloromethane (5 mL×3). The combined organic phases were concentrated and the residue was purified by preparative HPLC on a YMC-Actus Triart C18 ExRS column using a 17-42% gradient of acetonitrile in water (+10 mmol/L ammonium bicarbonate) to afford 4-{2-[({4-[2-(2-aminopyridin-3-yl)-5-cyclopropylimidazo[4,5-b]pyridin-3-yl]phenyl}methyl)amino]ethyl}-2-hydroxybenzaldehyde (4-{2-[({4-[2-(2-aminopyridin-3-yl)-5-cyclopropylimidazo[4,5-b]pyridin-3-yl]phenyl}methyl)amino]ethyl}-2-hydroxybenzaldehyde (Example 16) (5.1 mg, 7%) as an off-white solid. MS (ESI) calculated for C₃₀H₂₈N₆O₂: 504.23 m/z, found 505.20 [M+H]⁺. ¹H NMR (300 MHz, DMSO-d₆) δ (ppm): 10.15 (s, 1H), 8.01-8.04 (m, 1H), 7.94-7.96 (m, 1H), 7.57-7.60 (m, 1H), 7.31-7.45 (m, 2H), 7.23-7.28 (m, 3H), 7.07-7.10 (s, 1H), 6.82-6.85 (s, 2H), 6.30-6.34 (m, 1H), 3.78 (m, 2H), 2.74 (m, 4H), 2.13-2.18 (m, 1H), 0.78-0.95 (m, 4H).

### Intermediate 16-2: 3-(3-(4-(aminomethyl)phenyl)-5-cyclopropyl-3H-imidazo[4,5-b]pyridin-2-yl)pyridin-2-amine

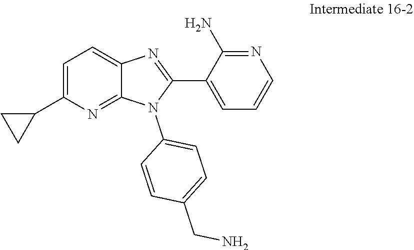 [SMILES: NCc1ccc(-n2c(-c3cccnc3N)nc3ccc(C4CC4)nc32)cc1]

Intermediate 16-2 was prepared in a manner analogous to Intermediate 11-1 using Intermediate 16-1 in place of 2-chloro-3-nitro-6-phenylpyridine and tert-butyl N-[(4-aminophenyl)methyl]carbamate in place of tert-butyl 2-(4-aminophenyl)acetate. MS (ESI) calculated for C₂₁H₂₀N₆: 356.17 m/z, found 357.35 [M+H]⁺.

### Intermediate 16-1: 2-chloro-6-cyclopropyl-3-nitropyridine

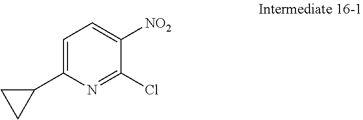 [SMILES: O=[N+]([O-])c1ccc(C2CC2)nc1Cl]

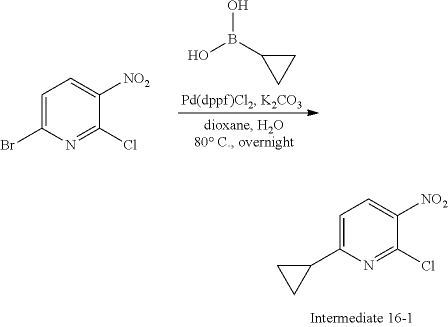 [SMILES: O=[N+]([O-])c1ccc(Br)nc1Cl ; OB(O)C1CC1 ; O=[N+]([O-])c1ccc(C2CC2)nc1Cl]

### Step 1: Synthesis of 2-chloro-6-cyclopropyl-3-nitropyridine (Intermediate 16-1)

To a stirred solution of 6-bromo-2-chloro-3-nitropyridine (4.00 g, 16.8 mmol, 1 equiv) in 1,4-dioxane (40 mL) and water (12 mL) were added cyclopropylboronic acid (2.46 g, 28.6 mmol, 1.7 equiv), [1,1′-bis(diphenylphosphino)ferrocene]dichloropalladium(II) (0.62 g, 0.84 mmol, 0.05 equiv) and potassium carbonate (4.66 g, 33.7 mmol, 2 equiv). The reaction mixture was stirred overnight at 80° C. under nitrogen atmosphere then cooled to room temperature and quenched with water. The resulting mixture was extracted with ethyl acetate (3×60 mL) and the combined organic layers were washed with brine (40 mL), dried over anhydrous sodium sulfate, filtered, and concentrated under reduced pressure. The residue was purified by silica gel column chromatography using a 0-100% gradient of ethyl acetate in petroleum ether to afford 2-chloro-6-cyclopropyl-3-nitropyridine (Intermediate 16-1) (2.7 g, 81%) as a red solid. MS (ESI) calculated for C₈H₇ClN₂O₂: 198.02 m/z, found 199.11 [M+H]⁺.

### Intermediate 16-4: 2-(3-((tert-butyldimethylsilyl)oxy)-4-(1,3-dioxolan-2-yl)phenyl) acetaldehyde

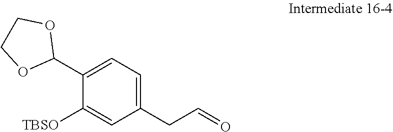 [SMILES: CC(C)(C)[Si](C)(C)Oc1cc(CC=O)ccc1C1OCCO1]

### Synthetic Route:

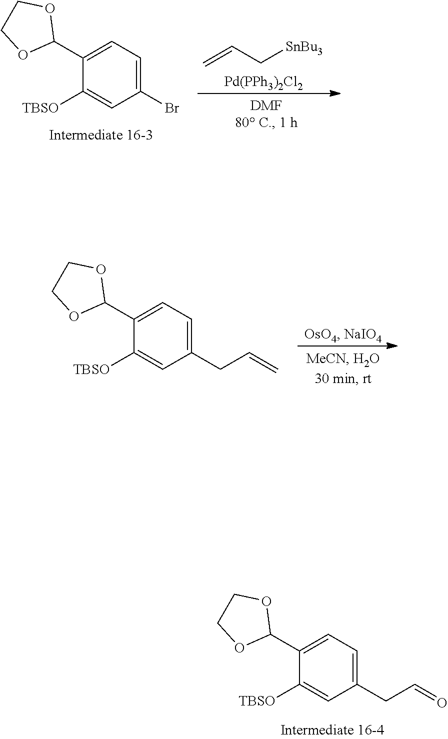

### Step 1: Synthesis of tert-butyl(2-(1,3-dioxolan-2-yl)-5-(prop-2-en-1-yl)phenoxy) dimethylsilane

To a solution of 5-bromo-2-(1,3-dioxolan-2-yl)phenoxy(tert-butyl)dimethylsilane (Intermediate 16-3) (1.00 g, 2.78 mmol, 1 equiv) and tributyl(prop-2-en-1-yl)stannane (1.84 g, 5.57 mmol, 2 equiv) in N,N-dimethylformamide (10 mL) was added bis(triphenylphosphine) palladium(II) dichloride (0.20 g, 0.28 mmol, 0.1 equiv). The resulting mixture was stirred for 1 h at 80° C. under nitrogen atmosphere then cooled to room temperature and concentrated in vacuo. The resulting residue was purified by reverse-phase flash column chromatography on C18 silica gel using a 10-40% gradient of acetonitrile in water to afford tert-butyl(2-(1,3-dioxolan-2-yl)-5-(prop-2-en-1-yl)phenoxy)dimethylsilane (0.8 g, 90%) as colorless oil. MS (ESI) calculated for C₁₈H₂₈O₃Si: 320.18 m/z, found 321.20 [M+H]⁺.

### Step 2: Synthesis of 2-{3-[(tert-butyldimethylsilyl)oxy]-4-(1,3-dioxolan-2-yl)phenyl}acetaldehyde (Intermediate 16-4)

To a solution of tert-butyl(2-(1,3-dioxolan-2-yl)-5-(prop-2-en-1-yl)phenoxy) dimethylsilane (1.00 g, 3.12 mmol, 1 equiv) in acetonitrile (3 mL) and water (3 mL) were added osmium tetroxide (2.38 g, 9.36 mmol, 3 equiv) and sodium periodate (2.00 g, 9.36 mmol, 3 equiv). After stirring for 30 min at room temperature, the reaction was quenched by addition of water. The resulting mixture was extracted with ethyl acetate (20 mL×3) and the combined organic layers were washed with water (20 mL×3), dried over anhydrous sodium sulfate, filtered, and concentrated under reduced pressure. The residue was purified by silica gel column chromatography using a 0-25% gradient of ethyl acetate in petroleum ether to provide 2-{3-[(tert-butyldimethylsilyl)oxy]-4-(1,3-dioxolan-2-yl)phenyl}acetaldehyde (0.2 g, 20%) as a light-yellow oil. MS (ESI) calculated for C₁₇H₂₆O₄Si: 322.16 m/z, found 323.15 [M+H]⁺.

### Intermediate 16-3: 5-bromo-2-(1,3-dioxolan-2-yl)phenoxy(tert-butyl)dimethylsilane

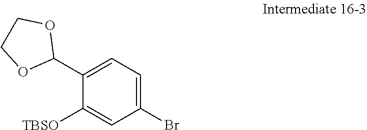 [SMILES: CC(C)(C)[Si](C)(C)Oc1cc(Br)ccc1C1OCCO1]

### Synthetic Route:

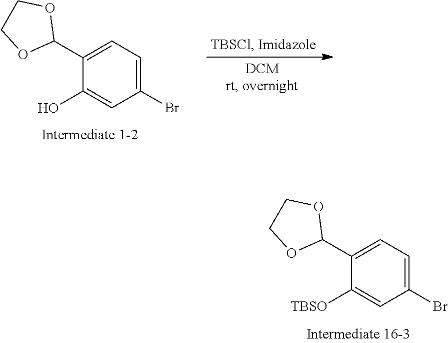 [SMILES: Oc1cc(Br)ccc1C1OCCO1 ; CC(C)(C)[Si](C)(C)Oc1cc(Br)ccc1C1OCCO1]

### Step 1: Synthesis of 5-bromo-2-(1,3-dioxolan-2-yl)phenoxy(tert-butyl)dimethylsilane (Intermediate 16-3)

To a solution of 5-bromo-2-(1,3-dioxolan-2-yl)phenol (Intermediate 1-2) (19.00 g, 77.53 mmol, 1 equiv) and imidazole (10.56 g, 155.1 mmol, 2 equiv) in dichloromethane (200 mL) was added tert-butylchlorodimethylsilane (16.36 g, 108.5 mmol, 1.4 equiv). The resulting mixture was stirred overnight at room temperature then cooled to 0° C. and quenched by the addition of water (200 mL). The resulting mixture was extracted with ethyl acetate (300 mL×3) and the combined organic layers were washed with water (300 mL×3), dried over anhydrous sodium sulfate, filtered, and concentrated in vacuo. The residue was purified by silica gel column chromatography using a 0-7% gradient of ethyl acetate in petroleum ether to provide 5-bromo-2-(1,3-dioxolan-2-yl)phenoxy(tert-butyl)dimethylsilane (Intermediate 16-3) (22 g, 79%) as a colorless oil. MS (ESI) calculated for C₁₅H₂₃BrO₃Si. 358.06 m/z, found 359.05 [M+H]⁺.

### Example 17: N-({4-[2-(2-aminopyridin-3-yl)-5-(2-hydroxypropan-2-yl)imidazo[4,5-b]pyridin-3-yl]phenyl}methyl)-2-(4-formyl-3-hydroxyphenyl)acetamide

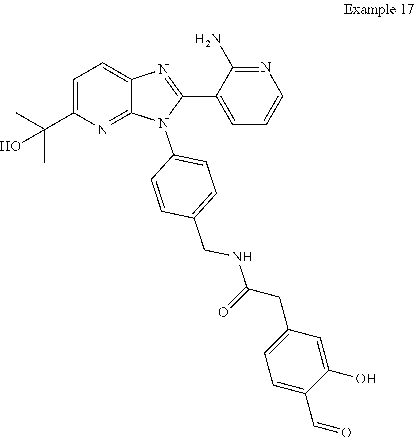 [SMILES: CC(C)(O)c1ccc2nc(-c3cccnc3N)n(-c3ccc(CNC(=O)Cc4ccc(C=O)c(O)c4)cc3)c2n1]

### Synthetic Route:

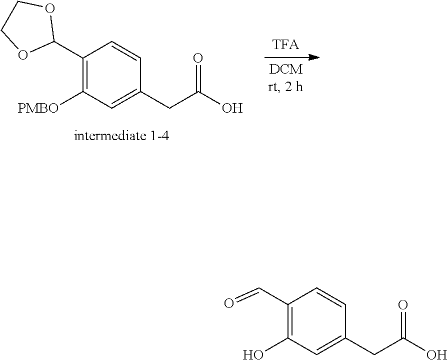

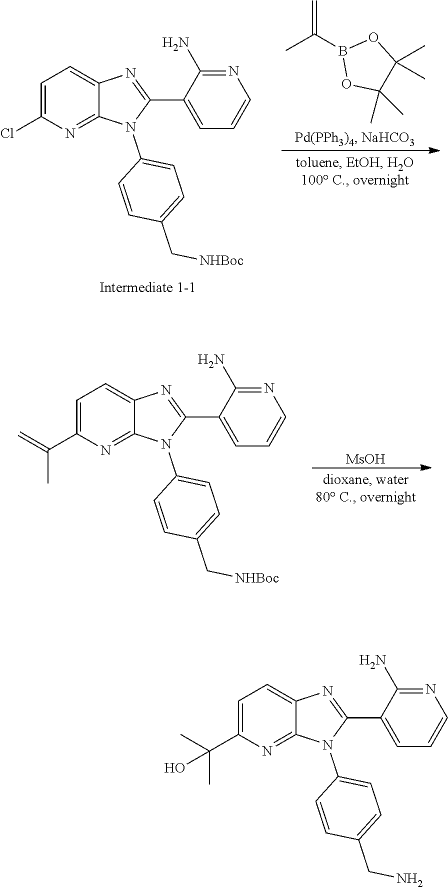

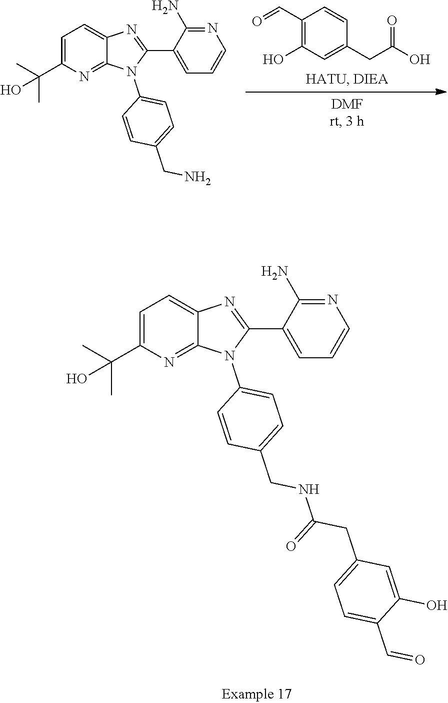 [SMILES: CC(C)(O)c1ccc2nc(-c3cccnc3N)n(-c3ccc(CN)cc3)c2n1 ; O=Cc1ccc(CC(=O)O)cc1O ; CC(C)(O)c1ccc2nc(-c3cccnc3N)n(-c3ccc(CNC(=O)Cc4ccc(C=O)c(O)c4)cc3)c2n1]

### Step 1: Synthesis of (4-formyl-3-hydroxyphenyl) acetic acid

A solution of 2-(4-(1,3-dioxolan-2-yl)-3-((4-methoxybenzyl)oxy)phenyl)acetic acid (Intermediate 1-4) (100 mg, 0.333 mmol, 1 equiv) and 2,2,2-trifluoroacetic acid (1 mL) in dichloromethane (2 mL) was stirred for 2 h at room temperature. The resulting mixture was concentrated under reduced pressure to afford (4-formyl-3-hydroxyphenyl) acetic acid (90 mg, crude quant.) as a white solid. MS (ESI) calculated for C₉H₈O₄: 180.04 m/z, found 181.05 [M+H]⁺.

### Step 2: Synthesis of tert-butyl N-({4-[2-(2-aminopyridin-3-yl)-5-(prop-1-en-2-yl)imidazo [4,5-b]pyridin-3-yl]phenyl}methyl)carbamate

A solution of tert-butyl N-({4-[2-(2-aminopyridin-3-yl)-5-chloroimidazo[4,5-b]pyridin-3-yl]phenyl}methyl)carbamate (Intermediate 1-1) (500 mg, 1.11 mmol, 1 equiv), 4,4,5,5-tetramethyl-2-(prop-1-en-2-yl)-1,3,2-dioxaborolane (745 mg, 4.44 mmol, 4 equiv), tetrakis(triphenylphosphine)palladium(0) (128 mg, 0.111 mmol, 0.1 equiv), cesium carbonate (373 mg, 4.44 mmol, 4 equiv) in toluene (1 mL), ethanol (5 mL) and water (5 mL) was stirred overnight at 100° C. under nitrogen atmosphere. The mixture was cooled to room temperature and quenched with water (50 mL). The resulting mixture was extracted with ethyl acetate (3×50 mL). The combined organic phases were concentrated under reduced pressure and the residue was purified by reverse-phase flash chromatography on C18 silica gel using a 10-50% gradient of acetonitrile in water (+0.05% 2,2,2-trifluoroacetic acid) to afford tert-butyl N-({4-[2-(2-aminopyridin-3-yl)-5-(prop-1-en-2-yl)imidazo[4,5-b]pyridin-3-yl]phenyl}methyl)carbamate (400 mg, 79%) as a black solid. MS (ESI) calculated for C₂₆H₂₈N₆O₂: 456.23 m/z, found 457.23 [M+H]⁺.

### Step 3 Synthesis of 2-{3-[4-(aminomethyl)phenyl]-2-(2-aminopyridin-3-yl)imidazo[4,5-b]pyridin-5-yl}propan-2-ol

A solution of tert-butyl N-({4-[2-(2-aminopyridin-3-yl)-5-(prop-1-en-2-yl)imidazo[4,5-b]pyridin-3-yl]phenyl}methyl)carbamate (1.00 g, 2.19 mmol, 1 equiv) and methanesulfonic acid (8 mL) in 1,4-dioxane (4 mL) and water (8 mL) was stirred overnight at 80° C. The mixture was cooled to room temperature and brough to pH 7 with ammonium bicarbonate. The aqueous layer was extracted with ethyl acetate (3×50 mL) and the combined organic phases were concentrated under reduced pressure and purified by reverse-phase flash chromatography on C18 silica gel using a 10-50% gradient of acetonitrile in water (+0.05% ammonium bicarbonate) to provide 2-{3-[4-(aminomethyl)phenyl]-2-(2-aminopyridin-3-yl)imidazo[4,5-b]pyridin-5-yl}propan-2-ol (160 mg, 20%) as a white solid. MS (ESI) calculated for C₂₁H₂₂N₆O: 374.19 m/z, found 375.19 [M+H]⁺.

### Step 4: Synthesis of N-({4-[2-(2-aminopyridin-3-yl)-5-(2-hydroxypropan-2-yl)imidazo[4,5-b]pyridin-3-yl]phenyl}methyl)-2-(4-formyl-3-hydroxyphenyl)acetamide (Example 17)

A solution of 2-(3-(4-(aminomethyl)phenyl)-2-(2-aminopyridin-3-yl)-3H-imidazo[4,5-b]pyridin-5-yl)propan-2-ol (100 mg, 0.267 mmol, 1 equiv), (4-formyl-3-hydroxyphenyl)acetic acid (vide supra) (48 mg, 0.27 mmol, 1 equiv), HATU (102 mg, 0.267 mmol, 1 equiv) and N,N-diisopropylethylamine (69 mg, 0.53 mmol, 2 equiv) in N,N-dimethylformamide (3 mL) was stirred at room temperature for 3 h. The resulting mixture was concentrated under reduced pressure and purified by reverse-phase flash chromatography on C18 silica gel using a 10-50% gradient of acetonitrile in water (+0.05% 2,2,2-trifluoroacetic acid) to provide N-({4-[2-(2-aminopyridin-3-yl)-5-(2-hydroxypropan-2-yl)imidazo[4,5-b]pyridin-3-yl]phenyl}methyl)-2-(4-formyl-3-hydroxyphenyl)acetamide (Example 17) (89 mg, 61%) as a yellow solid. MS (ESI) calculated for C₃₀H₂₈N₆O₄ 536.22 m/z, found 537.22 [M+H]⁺. ¹H NMR (400 MHz, DMSO-d₆) δ (ppm): 10.17 (s, 1H), 8.18-8.20 (m, 1H), 8.02-8.04 (m, 1H), 7.73-7.75 (m, 1H), 7.59-7.65 (m, 2H), 7.34-7.41 (m, 4H), 6.93-6.95 (m, 1H), 6.89-6.91 (m, 1H), 6.71-6.74 (m, 1H), 4.36 (s, 2H), 3.54-3.59 (m, 2H) 1.39-1.45 (m, 6H).

### Example 18: N-({4-[2-(2-aminopyridin-3-yl)-5-(1-methylcyclopropyl)imidazo[4,5-b]pyridin 3-yl]phenyl}methyl)-2-(4-formyl-3-hydroxyphenyl)acetamide

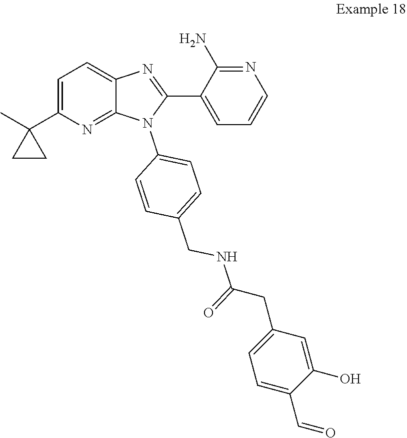 [SMILES: CC1(c2ccc3nc(-c4cccnc4N)n(-c4ccc(CNC(=O)Cc5ccc(C=O)c(O)c5)cc4)c3n2)CC1]

Example 18 was prepared in a manner analogous to Example 1 (starting from Step 3) using Intermediate 18-1 in place of the amine starting material. MS (ESI) calculated for C₃₁H₂₈N₆O₃: 532.22 m/z, found 533.25 [M+H]⁺. ¹H NMR (300 MHz, DMSO-d₆) δ (ppm): 10.18 (s, 1H), 8.06-8.09 (m, 1H), 7.97-7.99 (m, 1H), 7.60-7.62 (m, 1H), 7.33-7.39 (m, 5H), 7.12-7.16 (m, 1H), 6.89-6.95 (m, 2H), 6.38-6.42 (m, 1H), 4.37 (s, 2H), 3.61 (s, 2H), 1.49 (s, 3H), 1.05-1.06 (m, 2H), 0.77-0.79 (m, 2H).

### Intermediate 18-1: 3-(3-(4-(aminomethyl)phenyl)-5-(1-methylcyclopropyl)-3H-imidazo[4,5-b]pyridin-2-yl)pyridin-2-amine

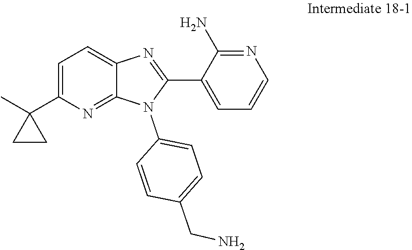 [SMILES: CC1(c2ccc3nc(-c4cccnc4N)n(-c4ccc(CN)cc4)c3n2)CC1]

### Synthetic Route:

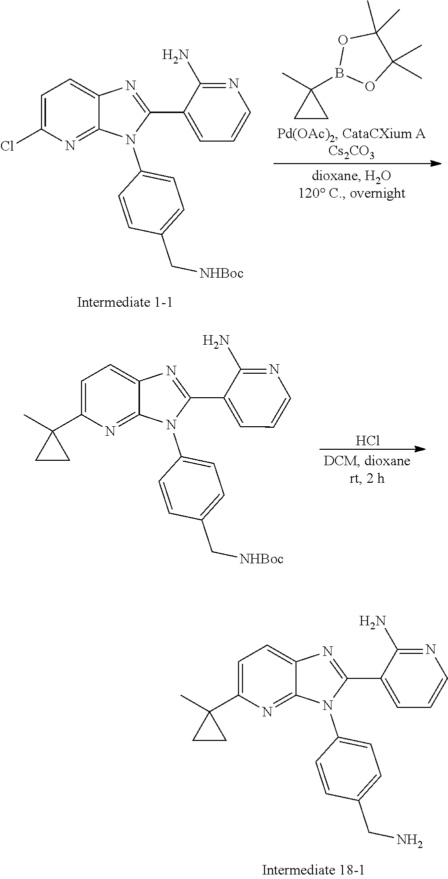

### Step 1: Synthesis of tert-butyl (4-(2-(2-aminopyridin-3-yl)-5-(1-methylcyclopropyl)-3H-imidazo [4,5-b]pyridin-3-yl)benzyl)carbamate

To a solution of tert-butyl N-({4-[2-(2-aminopyridin-3-yl)-5-chloroimidazo[4,5-b]pyridin-3-yl]phenyl}methyl)carbamate (Intermediate 1-1) (500 mg, 1.11 mmol, 1 equiv) in 1,4-dioxane (4 mL) and water (1 mL) were added 4,4,5,5-tetramethyl-2-(1-methylcyclopropyl)-1,3,2-dioxaborolane (114 mg, 1.33 mmol, 1.2 equiv), palladium(II) acetate (25 mg, 0.11 mmol, 0.1 equiv) and cataCXium A (CAS 321921-71-5) (80 mg, 0.22 mmol, 0.2 equiv). The resulting mixture was stirred at 120° C. overnight under nitrogen atmosphere. The mixture was then concentrated in vacuo and purified by reverse-phase flash column chromatography on C18 silica gel using a 20-95% gradient of acetonitrile in water (+0.05% ammonium bicarbonate) with a 10 minute hold at 70% acetonitrile to afford tert-butyl (4-(2-(2-aminopyridin-3-yl)-5-(1-methylcyclopropyl)-3H-imidazo[4,5-b]pyridin-3-yl)benzyl)carbamate (85 mg, 17%) as a yellow solid. MS (ESI) calculated for C₂₇H₃₀N₆O₂: 470.24 m/z, found 471.25 [M+H]⁺.

### Step 2: Synthesis of 3-(3-(4-(aminomethyl)phenyl)-5-(1-methylcyclopropyl)-3H-imidazo [4,5-b]pyridin-2-yl)pyridin-2-amine (Intermediate 18-1)

A solution of tert-butyl (4-(2-(2-aminopyridin-3-yl)-5-(1-methylcyclopropyl)-3H-imidazo[4,5-b]pyridin-3-yl)benzyl)carbamate (85 mg, 0.18 mmol, 1 equiv) in 4N hydrochloric acid in 1,4-dioxane (5 mL) was stirred at room temperature for 1 h. The solvent was removed under reduced pressure to afford 3-(3-(4-(aminomethyl)phenyl)-5-(1-methylcyclopropyl)-3H-imidazo[4,5-b]pyridin-2-yl)pyridin-2-amine (Intermediate 18-1) (60 mg, 71%) as a yellow/green solid, which was used without further purification in subsequent transformations. MS (ESI) calculated for C₂₂H₂₂N₆: 370.19 m/z, found 371.15 [M+H]⁺.

### Example 19: 4-(((4-(2-(2-aminopyridin-3-yl)-5-(3-morpholinophenyl)-3H-imidazo[4,5-b]pyridin-3-yl)phenethyl)amino)methyl)-2-hydroxybenzaldehyde

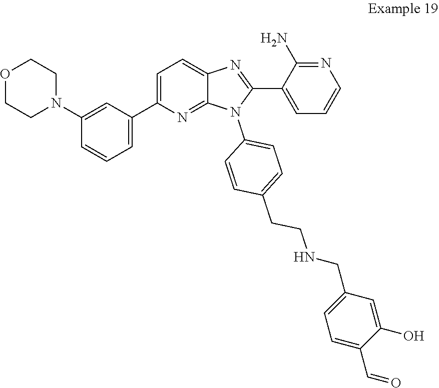 [SMILES: Nc1ncccc1-c1nc2ccc(-c3cccc(N4CCOCC4)c3)nc2n1-c1ccc(CCNCc2ccc(C=O)c(O)c2)cc1]

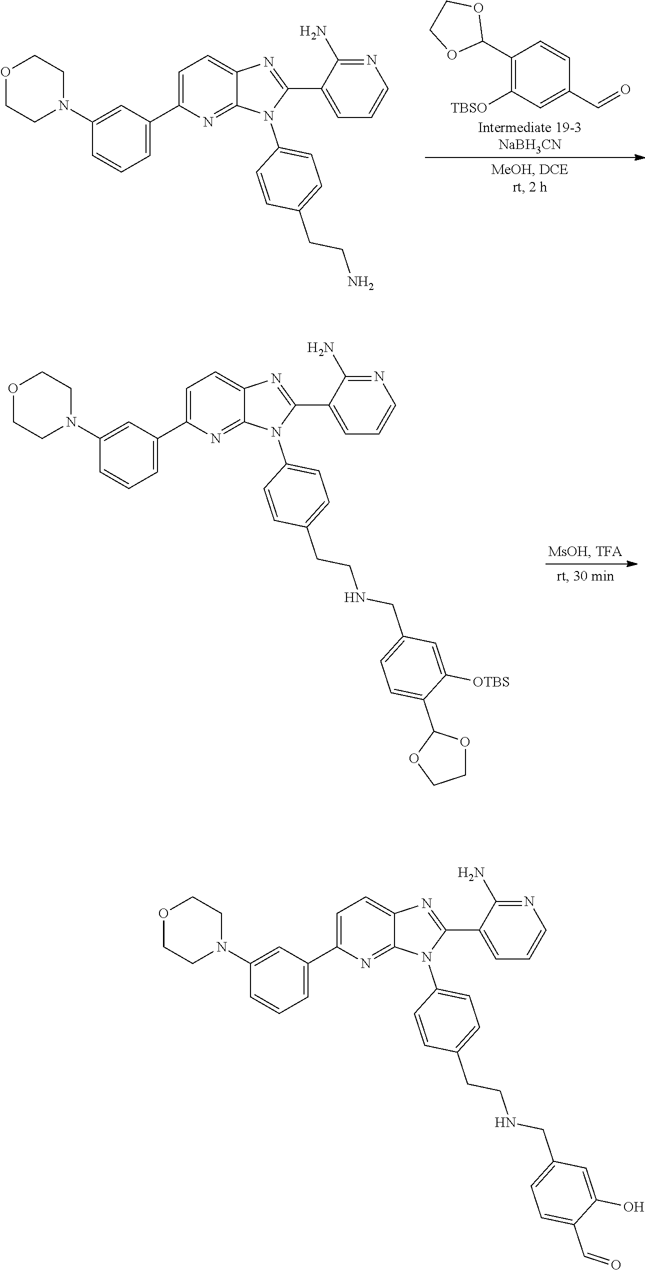 [SMILES: NCCc1ccc(-n2c(-c3cccnc3N)nc3ccc(-c4cccc(N5CCOCC5)c4)nc32)cc1 ; CC(C)(C)[Si](C)(C)Oc1cc(C=O)ccc1C1OCCO1 ; CC(C)(C)[Si](C)(C)Oc1cc(CNCCc2ccc(-n3c(-c4cccnc4N)nc4ccc(-c5cccc(N6CCOCC6)c5)nc43)cc2)ccc1C1OCCO1 ; Nc1ncccc1-c1nc2ccc(-c3cccc(N4CCOCC4)c3)nc2n1-c1ccc(CCNCc2ccc(C=O)c(O)c2)cc1]

### Step 1: Synthesis of 3-(3-(4-(2-((3-((tert-butyldimethylsilyl)oxy)-4-(1,3-dioxolan-2-yl) benzyl)amino)ethyl)phenyl)-5-(3-morpholinophenyl)-3H-imidazo[4,5-b]pyridin-2-yl) pyridin-2-amine

To a stirred solution of 3-{3-[4-(2-aminoethyl)phenyl]-5-[3-(morpholin-4-yl)phenyl]imidazo[4,5-b]pyridin-2-yl}pyridin-2-amine (Intermediate 19-2) (155 mg, 0.315 mmol, 1 equiv) in anhydrous methanol (2 mL) and 1,2-dichloroethane (2 mL) were added 3-[(tert-butyldimethylsilyl)oxy]-4-(1,3-dioxolan-2-yl)benzaldehyde (Intermediate 19-3) (82 mg, 0.26 mmol, 1 equiv) and sodium cyanoborohydride (33 mg, 0.53 mmol, 2 equiv) and the mixture was stirred at room temperature for 2 h. The mixture was quenched with water and extracted with dichloromethane (3×60 mL). The combined organic layers were washed with brine (40 mL), dried over anhydrous sodium sulfate, filtered, and concentrated in vacuo. The crude product was purified by reverse-phase flash column chromatography on C18 silica gel using a 20-60% gradient of acetonitrile in water to afford 3-(3-(4-(2-((3-((tert-butyldimethylsilyl)oxy)-4-(1,3-dioxolan-2-yl)benzyl)amino)ethyl)phenyl)-5-(3-morpholinophenyl)-3H-imidazo[4,5-b]pyridin-2-yl)pyridin-2-amine (80 mg, 39%) as a yellow solid. MS (ESI) calculated for C₄₅H₅₃N₇O₄Si: 783.39 m/z, found 784.41 [M+H]⁺.

### Step 2: Synthesis of 4-(((4-(2-(2-aminopyridin-3-yl)-5-(3-morpholinophenyl)-3H-imidazo[4,5-b]pyridin-3-yl)phenethyl)amino)methyl)-2-hydroxybenzaldehyde (Example 19)

To a stirred solution of 3-(3-(4-(2-((3-((tert-butyldimethylsilyl)oxy)-4-(1,3-dioxolan-2-yl)benzyl)amino)ethyl)phenyl)-5-(3-morpholinophenyl)-3H-imidazo[4,5-b]pyridin-2-yl)pyridin-2-amine (80 mg, 0.10 mmol, 1 equiv) in 2,2,2-trifluoroacetic acid (1 mL) was added methanesulfonic acid (0.3 mL) and the mixture was stirred at room temperature for 0.5 h. The reaction was quenched with water and the pH was brought to 8 with sodium bicarbonate. The resulting mixture was extracted with ethyl acetate (3×60 mL) and the combined organic layers were washed with brine (40 mL), dried over anhydrous sodium sulfate, filtered, and concentrated in vacuo. The crude product was purified by preparative HPLC on a XBridge Prep OBD C18 Column using a 26-45% gradient of acetonitrile in water (+10 mmol/L ammonium bicarbonate) to afford 4-(((4-(2-(2-aminopyridin-3-yl)-5-(3-morpholinophenyl)-3H-imidazo[4,5-b]pyridin-3-yl)phenethyl)amino)methyl)-2-hydroxybenzaldehyde (Example 19) (5.1 mg, 8%) as a yellow solid. MS (ESI) calculated for C₃₇H₃₅N₇O₃: 625.28 m/z, found 626.25 [M+H]⁺. ¹H NMR (300 MHz, DMSO-d₆) δ (ppm): 10.12 (s, 1H), 8.22-8.25 (m, 1H), 7.95-8.00 (m, 2H), 7.58-7.60 (m, 2H), 7.21-7.47 (m, 7H), 6.89-7.00 (m, 3H), 6.39-6.43 (s, 1H), 3.71 (m, 2H), 3.13-3.15 (m, 4H), 2.53-2.84 (m, 8H).

### Intermediate 19-2: 3-(3-(4-(2-aminoethyl)phenyl)-5-(3-morpholinophenyl)-3H-imidazo[4,5-b]pyridin-2-yl)pyridin-2-amine

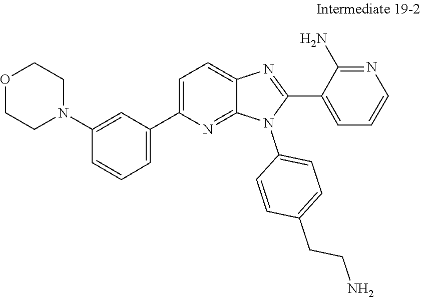 [SMILES: NCCc1ccc(-n2c(-c3cccnc3N)nc3ccc(-c4cccc(N5CCOCC5)c4)nc32)cc1]

Intermediate 19-2 was prepared in a manner analogous to Intermediate 12-1 using Intermediate 19-1 in place of Intermediate 1-1. MS (ESI) calculated for C₂₉H₂₉N₇O: 491.24 m/z, found 492.35 [M+H]⁺.

### Intermediate 19-1: tert-butyl (4-(2-(2-aminopyridin-3-yl)-5-chloro-3H-imidazo[4,5-b]pyridin-3-yl)phenethyl)carbamate

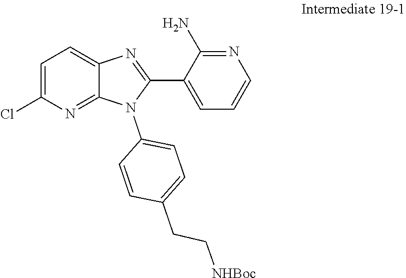 [SMILES: CC(C)(C)OC(=O)NCCc1ccc(-n2c(-c3cccnc3N)nc3ccc(Cl)nc32)cc1]

Intermediate 19-1 was prepared in a manner analogous to Intermediate 1-1 using tert-butyl N-[2-(4-aminophenyl)ethyl]carbamate in place of tert-butyl (4-aminobenzyl)carbamate. MS (ESI) calculated for C₂₄H₂₅ClN₆O₂: 464.17 m/z, found 465.16 [M+H]⁺.

### Intermediate 19-3: 4-(1,3-dioxolan-2-yl)-3-((4-methoxybenzyl)oxy)benzaldehyde

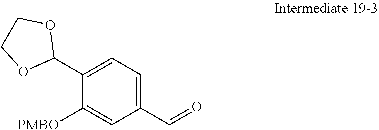

### Synthetic Route:

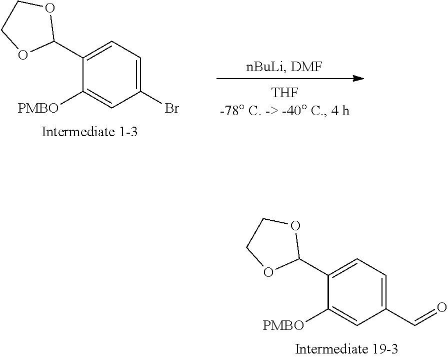

### Step 1: Synthesis of 4-(1,3-dioxolan-2-yl)-3-((4-methoxybenzyl)oxy)benzaldehyde (Intermediate 19-3)

To a cooled (−78° C.) suspension of 2-{4-bromo-2-[(4-methoxyphenyl)methoxy]phenyl}-1,3-dioxolane (Intermediate 1-4) (1.00 g, 2.74 mmol, 1 equiv) in tetrahydrofuran (20 mL) under nitrogen atmosphere was added dropwise n-butyllithium (2.5M in hexanes, 4.4 mL, 4 equiv). The obtained solution was stirred at −40° C. for 1 h. N,N-dimethylformamide (2 mL) was added dropwise and stirring was continued for 1 h at room temperature. The reaction was quenched by addition of saturated aqueous ammonium chloride (10 mL) and the mixture was extracted with ethyl acetate (10 mL×3). The combined organic phases were washed with brine (20 mL×2), dried with sodium sulfate, filtered, and concentrated in vacuo. The residue was purified by silica gel column chromatography using a gradient of 0-50% dichloromethane in petroleum ether to afford 4-(1,3-dioxolan-2-yl)-3-[(4-methoxyphenyl)methoxy]benzaldehyde (Intermediate 19-3) (669 mg, 51%) as a yellow oil. MS (ESI) calculated for C₁₈H₁₈O₅: 314.12 m/z, found 315.0 [M+H]⁺.

### Example 20: N-(4-(2-(2-aminopyridin-3-yl)-5-(3-morpholinophenyl)-3H-imidazo[4,5-b]pyridin-3-yl)benzyl)-3-formyl-4-hydroxybenzamide

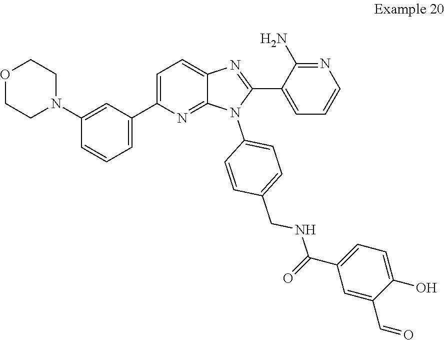 [SMILES: Nc1ncccc1-c1nc2ccc(-c3cccc(N4CCOCC4)c3)nc2n1-c1ccc(CNC(=O)c2ccc(O)c(C=O)c2)cc1]

Example 20 was prepared in a manner analogous to Example 1 (starting from Step 3) using Intermediate 12-1 in place of the amine starting material and Intermediate 3-4 in place of Intermediate 1-4. MS (ESI) calculated for C₃₆H₃₁N₇O₄: 625.24 m/z, found 626.30 [M+H]⁺. ¹H NMR (400 MHz, DMSO-d₆) δ 10.32 (s, 1H), 8.30-8.41 (m, 2H), 8.03-8.16 (m, 3H), 7.75-7.85 (m, 1H), 7.55-7.60 (m, 1H), 7.45-7.65 (m, 5H), 7.28-7.38 (m, 1H), 7.05-7.18 (m, 1H), 6.93-7.05 (m, 1H), 7.78-7.89 (m, 1H), 4.50-4.60 (m, 2H), 3.71-3.80 (m, 4H), 3.12-3.21 (m, 4H).

### Example 21: N-({4-[2-(2-aminopyridin-3-yl)-5-[3-(morpholin-4-yl)phenyl]imidazo[4,5-b]pyridin-3-yl]phenyl}methyl)-2-(2-fluoro-4-formyl-3-hydroxyphenyl)acetamide

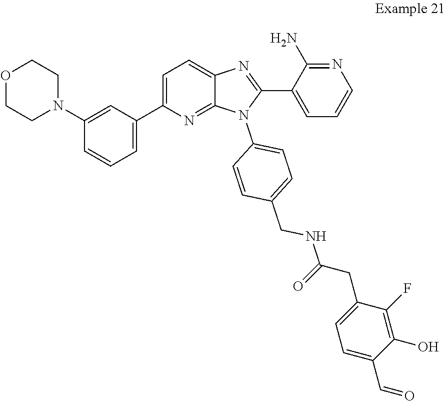 [SMILES: Nc1ncccc1-c1nc2ccc(-c3cccc(N4CCOCC4)c3)nc2n1-c1ccc(CNC(=O)Cc2ccc(C=O)c(O)c2F)cc1]

Example 21 was prepared in a manner analogous to Example 1 (starting from Step 3) using Intermediate 12-1 in place of the amine starting material and Intermediate 21-2 in place of Intermediate 1-4. MS (ESI) calculated for C₃₇H₃₂FN₇O₄: 657.25 m/z, found 658.25 [M+H]⁺. ¹H NMR (400 MHz, DMSO-d₆) δ (ppm): 10.18 (s, 1H), 8.30-8.32 (m, 1H), 8.03-8.05 (m, 2H), 7.81-7.82 (m, 1H), 7.42-7.58 (m, 7H), 7.31-7.35 (m, 1H), 6.95-7.02 (m, 2H), 6.85-6.92 (m, 1H), 4.39 (s, 2H), 3.76-3.82 (m, 6H), 3.15-3.16 (m, 4H). ¹⁹F NMR (400 MHz, DMSO-d₆) δ (ppm): 139.31.

### Intermediate 21-2: 2-(4-(1,3-dioxolan-2-yl)-2-fluoro-3-((4-methoxybenzyl)oxy)phenyl)acetic acid

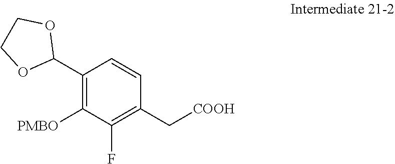

Intermediate 21-2 was prepared in a manner analogous to Intermediate 1-4 using Intermediate 21-1 in place of Intermediate 1-3. MS (ESI) calculated for C₁₉H₁₉FO₆: 362.12 m/z, found 363.12 [M+H]⁺.

### Intermediate 21-1: 2-(4-bromo-3-fluoro-2-((4-methoxybenzyl)oxy)phenyl)-1,3-dioxolane

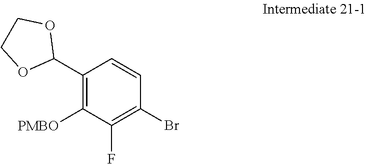

Intermediate 21-1 was prepared in a manner analogous to Intermediate 1-3 (via Intermediate 1-2) starting from 3-bromo-2-fluorophenol in place of 4-bromo-2-hydroxybenzaldehyde. MS (ESI) calculated for C₁₇H₁₆BrFO₄: 382.02 m/z, found 381.00 [M−H]⁻

### Example 22: N-({4-[2-(2-aminopyridin-3-yl)-5-cyclopropylimidazo[4,5-b]pyridin-3-yl]phenyl}methyl)-2-(2-chloro-4-formyl-3-hydroxyphenyl)acetamide

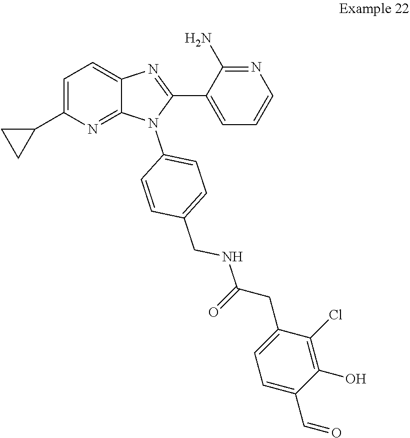 [SMILES: Nc1ncccc1-c1nc2ccc(C3CC3)nc2n1-c1ccc(CNC(=O)Cc2ccc(C=O)c(O)c2Cl)cc1]

Example 22 was prepared in a manner analogous to Example 1 (starting from Step 3) using Intermediate 16-2 in place of the amine starting material and Intermediate 13-2 in place of Intermediate 1-4. MS (ESI) calculated for C₃₀H₂₅ClN₆O₃: 552.02 m/z, found 553.20 [M+H]⁺. ¹H NMR (400 MHz, DMSO-d₆) δ (ppm): 10.95 (s, 1H), 8.01-8.11 (m, 2H), 7.59-7.75 (m, 2H), 7.35-7.39 (m, 4H), 7.29-7.34 (m, 1H), 7.05-7.15 (m, 1H), 6.71-6.79 (m, 1H), 4.45 (s, 2H), 3.80 (s, 2H), 2.15-2.26 (m, 1H), 0.92-1.01 (m, 2H), 0.79-0.88 (m, 2H).

### Example 23: N-({4-[2-(2-aminopyridin-3-yl)-5-isopropylimidazo[4,5-b]pyridin-3-yl]phenyl}methyl)-2-(4-formyl-3-hydroxyphenyl)acetamide

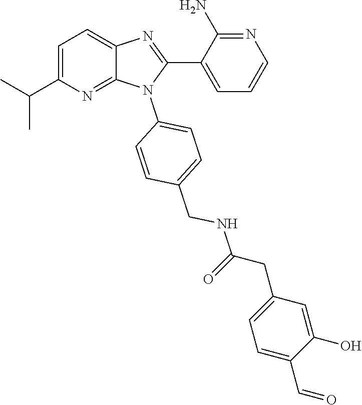 [SMILES: CC(C)c1ccc2nc(-c3cccnc3N)n(-c3ccc(CNC(=O)Cc4ccc(C=O)c(O)c4)cc3)c2n1]

Example 23 was prepared in a manner analogous to Example 1 (starting from Step 3) using Intermediate 23-1 in place of the amine starting material. MS (ESI) calculated for C₃₀H₂₈N₆O₃: 520.22 m/z, found 521.25 [M+H]⁺. ¹H NMR (400 MHz, DMSO-d₆) δ (ppm): 10.22 (s, 1H), 8.10-8.20 (m, 1H), 7.85-8.02 (m, 1H), 7.50-7.70 (m, 1H), 7.40 (s, 4H), 7.20-7.35 (m, 1H), 7.10-7.20 (m, 1H), 6.92-7.00 (m, 1H), 6.80-6.92 (m, 1H), 6.25-6.45 (m, 1H), 4.30-4.50 (m, 2H), 3.50-3.70 (m, 2H), 3.00-3.15 (m, 1H), 1.15-1.25 (m, 6H).

### Intermediate 23-1: 3-(3-(4-(aminomethyl)phenyl)-5-isopropyl-3H-imidazo[4,5-b]pyridin-2-yl)pyridin-2-amine

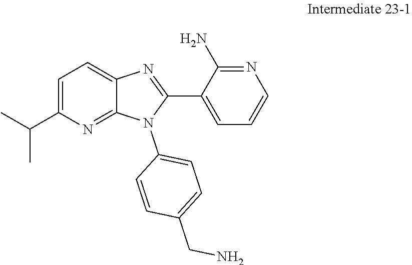 [SMILES: CC(C)c1ccc2nc(-c3cccnc3N)n(-c3ccc(CN)cc3)c2n1]

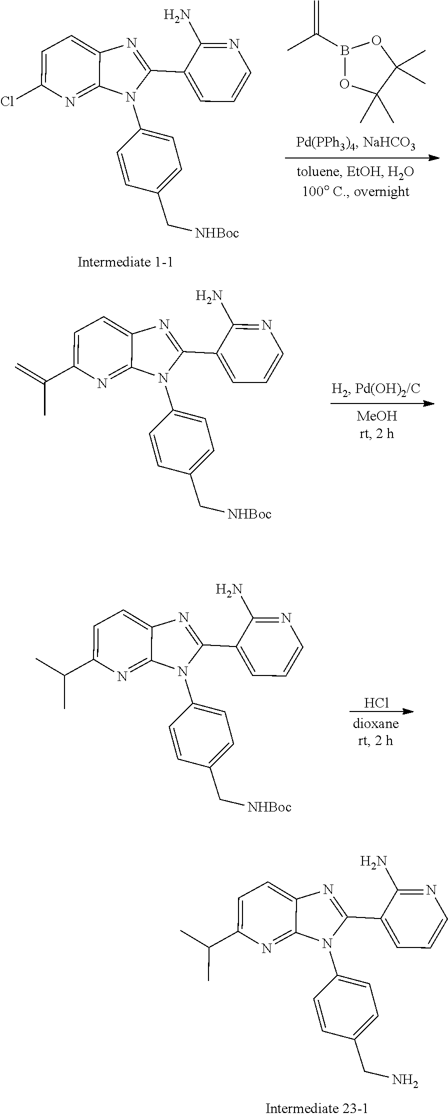

### Step 1: Synthesis of tert-butyl N-({4-[2-(2-aminopyridin-3-yl)-5-(prop-1-en-2-yl)imidazo [4,5-b]pyridin-3-yl]phenyl}methyl)carbamate

A solution of tert-butyl N-({4-[2-(2-aminopyridin-3-yl)-5-chloroimidazo[4,5-b]pyridin-3-yl]phenyl}methyl)carbamate (Intermediate 1-1) (500 mg, 1.11 mmol, 1 equiv), 4,4,5,5-tetramethyl-2-(prop-1-en-2-yl)-1,3,2-dioxaborolane (745 mg, 4.44 mmol, 4 equiv), tetrakis(triphenylphosphine)palladium(0) (128 mg, 0.111 mmol, 0.1 equiv), cesium carbonate (373 mg, 4.44 mmol, 4 equiv) in toluene (1 mL), ethanol (5 mL) and water (5 mL) was stirred overnight at 100° C. under nitrogen atmosphere. The mixture was cooled to room temperature and quenched with water (50 mL). The resulting mixture was extracted with ethyl acetate (3×50 mL). The combined organic phases were concentrated under reduced pressure and the residue was purified by reverse-phase flash chromatography on C18 silica gel using a 10-50% gradient of acetonitrile in water (+0.05% 2,2,2-trifluoroacetic acid) to afford tert-butyl N-({4-[2-(2-aminopyridin-3-yl)-5-(prop-1-en-2-yl)imidazo[4,5-b]pyridin-3-yl]phenyl}methyl)carbamate (400 mg, 79%) as a black solid. MS (ESI) calculated for C₂₆H₂₈N₆O₂: 456.23 m/z, found 457.23 [M+H]⁺.

### Step 2: Synthesis of tert-butyl N-({4-[2-(2-aminopyridin-3-yl)-5-isopropylimidazo[4,5-b]pyridin-3-yl]phenyl}methyl)carbamate

To a solution of tert-butyl N-({4-[2-(2-aminopyridin-3-yl)-5-(prop-1-en-2-yl)imidazo[4,5-b]pyridin-3-yl]phenyl}methyl)carbamate (214 mg, 0.469 mmol, 1 equiv) in methanol (10 mL) was added Pd(OH)₂/C (10%, 22 mg, 0.05 equiv). The mixture was stirred at room temperature under hydrogen atmosphere for 2 h. The mixture was then filtered and concentrated in vacuo to afford tert-butyl N-({4-[2-(2-aminopyridin-3-yl)-5-isopropylimidazo[4,5-b]pyridin-3-yl]phenyl}methyl)carbamate (120 mg, 55%) as a yellow solid, which was used without further purification in the next step. MS (ESI) calculated for C₂₆H₃₀N₆O₂: 458.24 m/z, found 459.15 [M+H]⁺.

### Step 3: Synthesis of 3-{3-[4-(aminomethyl)phenyl]-5-isopropylimidazo[4,5-b]pyridin-2-yl}pyridin-2-amine (Intermediate 23-1)

tert-Butyl N-({4-[2-(2-aminopyridin-3-yl)-5-isopropylimidazo[4,5-b]pyridin-3-yl]phenyl}methyl)carbamate (120 mg, 0.262 mmol, 1 equiv) was dissolved in 4N hydrochloric acid in 1,4-dioxane (5 mL) and the mixture was stirred at room temperature for 1 h. The mixture was concentrated in vacuo to afford 3-{3-[4-(aminomethyl)phenyl]-5-isopropylimidazo[4,5-b]pyridin-2-yl}pyridin-2-amine (Intermediate 23-1) (95 mg, 95%) as a yellow solid, which was used in subsequent transformations without further purification. MS (ESI) calculated for C₂₁H₂₂N₆: 358.19 m/z, found 359.35 [M+H]⁺.

### Example 24: N-({4-[2-(2-aminopyridin-3-yl)-5-methylimidazo[4,5-b]pyridin-3-yl]phenyl}methyl)-2-(4-formyl-3-hydroxyphenyl)acetamide

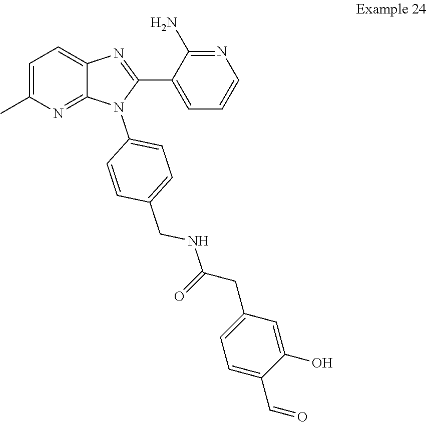 [SMILES: Cc1ccc2nc(-c3cccnc3N)n(-c3ccc(CNC(=O)Cc4ccc(C=O)c(O)c4)cc3)c2n1]

Example 24 was prepared in a manner analogous to Example 1 (starting from Step 3) using Intermediate 24-2 in place of the amine starting material. MS (ESI) calculated for C₂₈H₂₄N₆O₃: 492.19 m/z, found 493.20 [M+H]⁺. ¹H NMR (400 MHz, DMSO-d₆) δ (ppm): 10.17 (s, 1H), 8.05-8.07 (m, 1H), 7.95-7.96 (m, 1H), 7.55-7.61 (m, 1H), 7.35-7.38 (m, 4H), 7.32-7.34 (m, 1H), 7.24-7.26 (m, 1H), 6.94-7.174 (m, 2H), 6.36-6.39 (m, 1H), 4.35-4.36 (m, 2H), 3.51-3.60 (m, 2H), 2.52-2.54 (m, 3H).

### Intermediate 24-2: 3-(3-(4-(aminomethyl)phenyl)-5-methyl-3H-imidazo[4,5-b]pyridin-2-yl)pyridin-2-amine

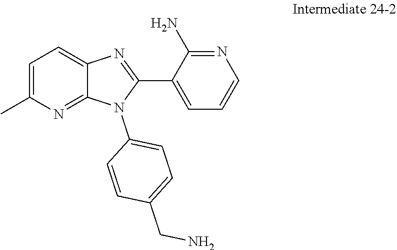 [SMILES: Cc1ccc2nc(-c3cccnc3N)n(-c3ccc(CN)cc3)c2n1]

### Synthetic Route:

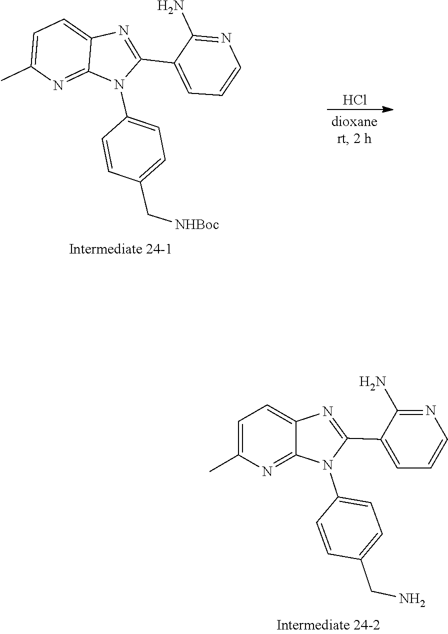 [SMILES: Cc1ccc2nc(-c3cccnc3N)n(-c3ccc(CNC(=O)OC(C)(C)C)cc3)c2n1 ; Cl ; Cc1ccc2nc(-c3cccnc3N)n(-c3ccc(CN)cc3)c2n1]

### Step 1: Synthesis of 3-{3-[4-(aminomethyl)phenyl]-5-methylimidazo[4,5-b]pyridin-2-yl}pyridin-2-amine (Intermediate 24)

A mixture of tert-butyl N-({4-[2-(2-aminopyridin-3-yl)-5-methylimidazo[4,5-b]pyridin-3-yl]phenyl}methyl)carbamate (Intermediate 24-1) (172 mg, 0.40 mmol, 1 equiv) in 4N hydrochloric acid in 1,4-dioxane (10 mL) was stirred at room temperature for 2 h. The mixture was concentrated in vacuo to afford 3-{3-[4-(aminomethyl)phenyl]-5-methylimidazo[4,5-b]pyridin-2-yl}pyridin-2-amine (Intermediate 24-2) (150 mg, crude quant.) as a yellow solid, which was used without further purification in subsequent transformations. MS (ESI) calculated for C₁₉H₁₈N₆: 330.21 m/z, found 331.21 [M+H]⁺.

### Intermediate 24-1: tert-butyl (4-(2-(2-aminopyridin-3-yl)-5-methyl-3H-imidazo[4,5-b]pyridin-3-yl)benzyl)carbamate

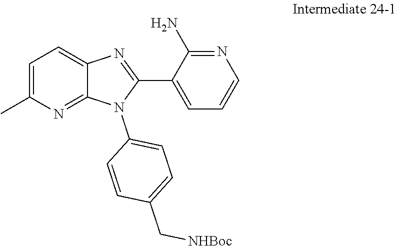 [SMILES: Cc1ccc2nc(-c3cccnc3N)n(-c3ccc(CNC(=O)OC(C)(C)C)cc3)c2n1]

Intermediate 24-1 was prepared in a manner analogous to Intermediate 1-1 using 2-chloro-6-methyl-3-nitropyridine in place of 2,6-dichloro-3-nitropyridine. MS (ESI) calculated for C₂₄H₂₆N₆O₂: 430.21 m/z, found 431.20 [M+H]⁺.

### Example 25: N-({4-[2-(2-aminopyridin-3-yl)-5-(cyclopropylamino)imidazo[4,5-b]pyridin-3-yl]phenyl}methyl)-2-(4-formyl-3-hydroxyphenyl)acetamide

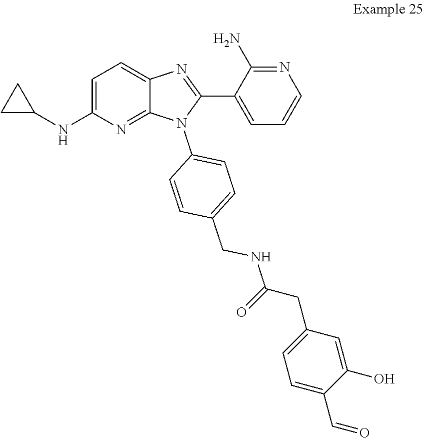 [SMILES: Nc1ncccc1-c1nc2ccc(NC3CC3)nc2n1-c1ccc(CNC(=O)Cc2ccc(C=O)c(O)c2)cc1]

Example 25 was prepared in a manner analogous to Example 1 (starting from Step 3) using Intermediate 25-1 in place of the amine starting material. MS (ESI) calculated for C₃₀H₂₇N₇O₃: 533.22 m/z, found 534.20 [M+H]⁺. ¹H NMR (300 MHz, DMSO-d₆) δ ppm: 10.18 (s, 1H), 7.88-7.91 (m, 2H), 7.55-7.71 (m, 1H), 7.26-7.40 (m, 4H), 7.00-7.06 (m, 1H), 6.90-6.94 (m, 1H), 6.88-6.92 (m, 1H), 6.68-6.75 (m, 1H), 6.29-6.33 (m, 1H), 4.34 (s, 2H), 3.53-3.55 (m, 2H), 2.44-2.47 (m, 1H), 0.66-0.67 (m, 2H), 0.40-0.41 (m, 2H).

### Intermediate 25-1: 3-(4-(aminomethyl)phenyl)-2-(2-aminopyridin-3-yl)-N-cyclopropyl-3H-imidazo[4,5-b]pyridin-5-amine

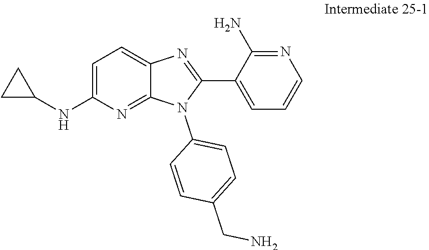 [SMILES: NCc1ccc(-n2c(-c3cccnc3N)nc3ccc(NC4CC4)nc32)cc1]

### Synthetic Route:

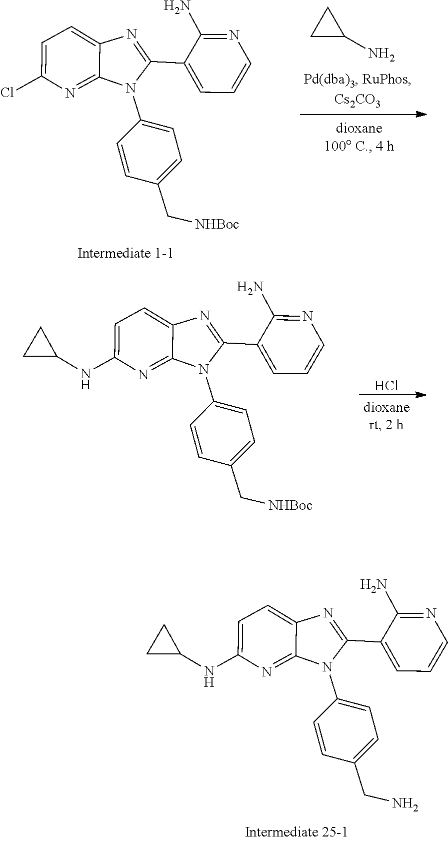

### Step 1: Synthesis of tert-butyl (4-(2-(2-aminopyridin-3-yl)-5-(cyclopropylamino)-3H-imidazo[4,5-b]pyridin-3-yl)benzyl)carbamate

To a solution of tert-butyl (4-(2-(2-aminopyridin-3-yl)-5-chloro-3H-imidazo[4,5-b]pyridin-3-yl)benzyl)carbamate (Intermediate 1-1) (252 mg, 0.541 mmol, 1 equiv) in 1,4-dioxane (5 mL) were added aminocyclopropane (154 mg, 2.71 mmol, 5 equiv), tris(dibenzylideneacetone)dipalladium(0) (50 mg, 0.054 mmol, 0.1 equiv), RuPhos (50 mg, 0.11 mmol, 0.2 equiv) and cesium carbonate (441 mg, 1.35 mmol, 2.5 equiv). The resulting mixture was stirred for 4 h at 100° C. under a nitrogen atmosphere, then cooled to room temperature and concentrated in vacuo. The residue was purified by reverse-phase flash column chromatography on C18 silica gel using a 0-40% gradient of acetonitrile in water to afford tert-butyl (4-(2-(2-aminopyridin-3-yl)-5-(cyclopropylamino)-3H-imidazo[4,5-b]pyridin-3-yl)benzyl)carbamate (40 mg, 15%) as a yellow solid. MS (ESI) calculated for C₂₆H₂₉N₇O₂: 471.24 m/z, found 472.40 [M+H]⁺.

### Step 2: Synthesis of 3-{3-[4-(aminomethyl)phenyl]-5-(cyclopropylamino)imidazo[4,5-b]pyridin-2-yl}pyridin-2-amine (Intermediate 25-1)

A solution of tert-butyl N-({4-[2-(2-aminopyridin-3-yl)-5-(cyclopropylamino)imidazo[4,5-b]pyridin-3-yl]phenyl}methyl)carbamate (47 mg, 0.10 mmol, 1 equiv) in 4N hydrochloric acid in 1,4-dioxane was stirred at room temperature for 1 h. The resulting mixture was concentrated to afford 3-{3-[4-(aminomethyl)phenyl]-5-(cyclopropylamino)imidazo[4,5-b]pyridin-2-yl}pyridin-2-amine (Intermediate 25-1) (40 mg, 81%) as a light-yellow solid, which was used without further purification in subsequent transformations. MS (ESI) calculated for C₂₁H₂₁N₇: 371.19 m/z, found 372.20 [M+H]⁺.

### Example 26: N-({4-[2-(2-aminopyridin-3-yl)-5-cyclopropylimidazo[4,5-b]pyridin-3-yl]phenyl}methyl)-2-fluoro-2-(4-formyl-3-hydroxyphenyl)acetamide

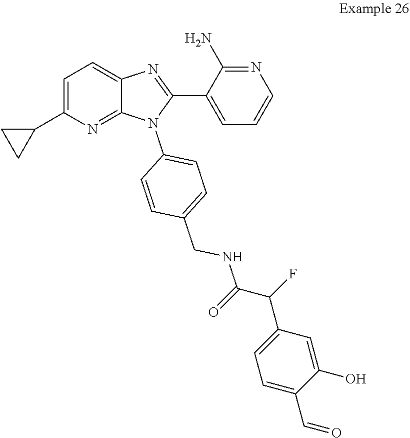 [SMILES: Nc1ncccc1-c1nc2ccc(C3CC3)nc2n1-c1ccc(CNC(=O)C(F)c2ccc(C=O)c(O)c2)cc1]

Example 26 was prepared in a manner analogous to Example 1 (starting from Step 3) using Intermediate 16-2 in place of the amine starting material and Intermediate 65-1 in place of Intermediate 1-4. MS (ESI) calculated for C₃₀H₂₅FN₆O₃: 536.20 m/z, found 537.20 [M+H]⁺. ¹H NMR (300 MHz, DMSO-d₆) δ (ppm): 10.26 (s, 1H), 9.20-9.23 (m, 1H), 8.07-8.09 (m, 1H), 8.00-8.02 (m, 1H), 7.70-7.72 (m, 1H), 7.57-7.59 (m, 1H), 7.30-7.39 (m, 5H), 7.13-7.20 (m, 1H), 7.05-7.07 (m, 1H), 6.67-6.70 (m, 1H), 5.97-6.09 (m, 1H), 4.41-4.50 (m, 2H), 2.17-2.21 (m, 1H), 0.97-0.98 (m, 2H), 0.95-0.96 (m, 2H). ¹⁹F NMR (400 MHz, DMSO-d₆) δ (ppm): −181.39.

### Example 27: N-({4-[2-(2-aminopyridin-3-yl)-5-cyclopropylimidazo[4,5-b]pyridin-3-yl]phenyl}methyl)-3-formyl-4-hydroxybenzamide

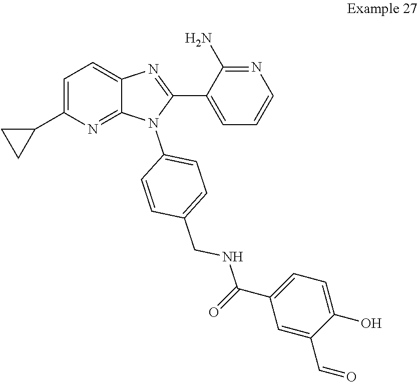 [SMILES: Nc1ncccc1-c1nc2ccc(C3CC3)nc2n1-c1ccc(CNC(=O)c2ccc(O)c(C=O)c2)cc1]

Example 27 was prepared in a manner analogous to Example 1 (starting from Step 3) using Intermediate 16-2 in place of the amine starting material and Intermediate 3-4 in place of Intermediate 1-4. MS (ESI) calculated for C₂₉H₂₄N₆O₃: 504.19 m/z, found 505.20 [M+H]⁺. ¹H NMR (400 MHz, DMSO-d₆) δ (ppm): 10.32 (s, 1H), 8.28-8.29 (m, 1H), 8.03-8.11 (m, 3H), 7.66-7.69 (m, 1H), 7.39-7.46 (m, 4H), 7.33-7.38 (m, 1H), 7.08-7.11 (m, 1H), 6.75-6.79 (m, 1H), 4.55 (s, 2H), 2.17-2.22 (m, 1H), 0.83-0.96 (m, 4H).

### Example 28: N-{4-[2-(2-aminopyridin-3-yl)-5-phenylimidazo[4,5-b]pyridin-3-yl]phenyl}-2-(3-formyl-4-hydroxyphenyl)acetamide

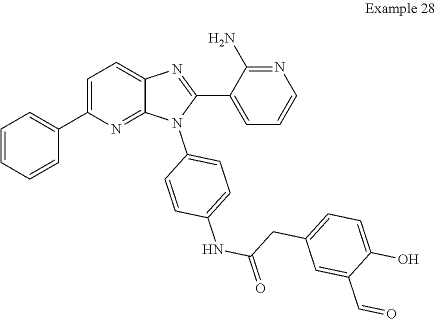 [SMILES: Nc1ncccc1-c1nc2ccc(-c3ccccc3)nc2n1-c1ccc(NC(=O)Cc2ccc(O)c(C=O)c2)cc1]

Example 28 was prepared in a manner analogous to Example 1 (starting from Step 3) using Intermediate 28-1 in place of the amine starting material and Intermediate 28-2 in place of Intermediate 1-4. MS (ESI) calculated for C₃₂H₂₄N₆O₃: 540.19 m/z, found 541.30 [M+H]⁺. ¹H-NMR (400 MHz, DMSO-d₆) δ (ppm): 10.22 (s, 1H), 8.29-8.38 (m, 1H), 8.01-8.09 (m, 4H), 7.75-7.81 (m, 2H), 7.59-7.68 (m, 2H), 7.35-7.52 (m, 5H), 6.95-7.01 (m, 2H), 6.69-6.78 (m, 1H), 3.70 (s, 2H).

### Intermediate 28-1: 3-(3-(4-aminophenyl)-5-phenyl-3H-imidazo[4,5-b]pyridin-2-yl)pyridin-2-amine

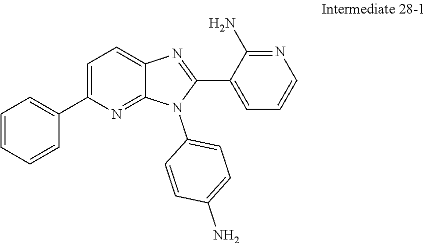 [SMILES: Nc1ccc(-n2c(-c3cccnc3N)nc3ccc(-c4ccccc4)nc32)cc1]

Intermediate 28-1 was prepared in a manner analogous to Intermediate 11-1 using tert-butyl N-(4-aminophenyl)carbamate in place of tert-butyl 2-(4-aminophenyl)acetate. MS (ESI) calculated for C₂₃H₁₈N₆: 378.16 m/z, found 379.15 [M+H]⁺.

### Intermediate 28-2: 2-(3-(1,3-dioxolan-2-yl)-4-((4-methoxybenzyl)oxy)phenyl)acetic acid

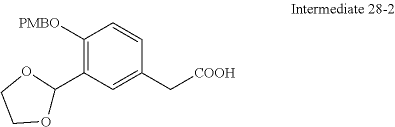

Intermediate 28-2 was prepared in a manner analogous to Intermediate 1-4 using Intermediate 3-3 in place of Intermediate 1-3. MS (ESI) calculated for C₁₉H₂₀O₆: 344.13 m/z, found 345.15 [M+H]⁺.

### Example 29: 4-(2-(2-aminopyridin-3-yl)-5-phenyl-3H-imidazo[4,5-b]pyridin-3-yl)-N-(3-formyl-4-hydroxybenzyl)benzamide

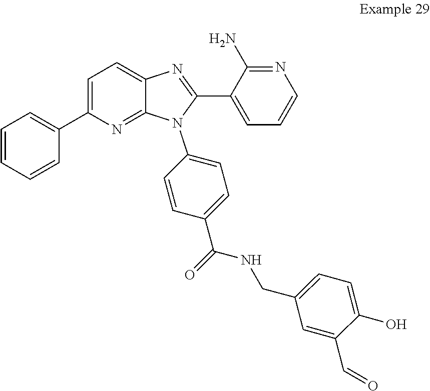 [SMILES: Nc1ncccc1-c1nc2ccc(-c3ccccc3)nc2n1-c1ccc(C(=O)NCc2ccc(O)c(C=O)c2)cc1]

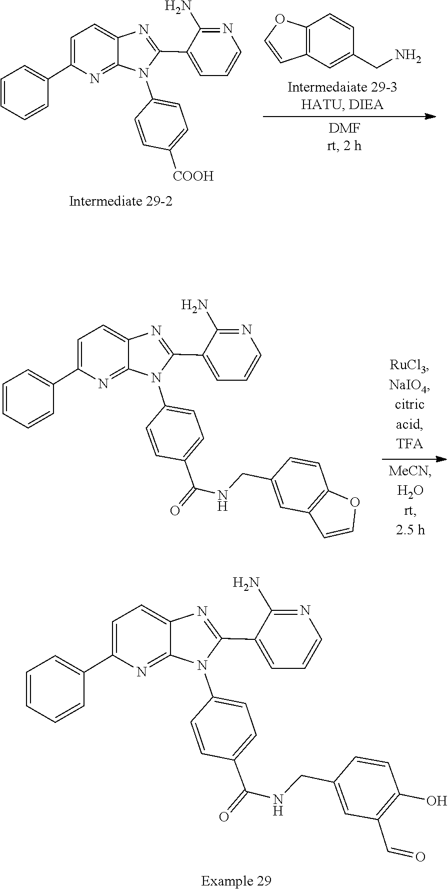

### Step 1: Synthesis of 4-(2-(2-aminopyridin-3-yl)-5-phenyl-3H-imidazo[4,5-b]pyridin-3-yl)-N-(benzofuran-6-ylmethyl)benzamide

To a solution of 4-[2-(2-aminopyridin-3-yl)-5-phenylimidazo[4,5-b]pyridin-3-yl]benzoic acid (Intermediate 29-2) (85 mg, 0.21 mmol, 1 equiv), 1-(1-benzofuran-5-yl)methanamine (Intermediate 29-3) (37 mg, 0.25 mmol, 1.2 equiv) and N,N-diisopropylethylamine (162 mg, 1.25 mmol, 6 equiv) in N,N-dimethylformamide (2 mL) was added HATU (119 mg, 0.314 mmol, 1.5 equiv). The resulting mixture was stirred at room temperature for 2 h. The reaction was quenched by addition of water (10 mL) and the mixture was extracted with ethyl acetate (10 mL×3). The combined organic phases were washed with brine (30 mL×3), dried with sodium sulfate, filtered, and concentrated in vacuo. The residue was purified by silica gel column chromatography using a 0-10% gradient of dichloromethane in methanol to afford 4-[2-(2-aminopyridin-3-yl)-5-phenylimidazo[4,5-b]pyridin-3-yl]-N-(1-benzofuran-6-ylmethyl)benzamide (42 mg, 33%) as a yellow solid. MS (ESI) calculated for C₃₃H₂₄N₆O₂: 536.20 m/z, found 537.05 [M+H]⁺.

### Step 2: Synthesis of 4-(2-(2-aminopyridin-3-yl)-5-phenyl-3H-imidazo[4,5-b]pyridin-3-yl)-N-(3-formyl-4-hydroxybenzyl)benzamide (Example 29)

To a solution of 4-[2-(2-aminopyridin-3-yl)-5-phenylimidazo[4,5-b]pyridin-3-yl]-N-(1-benzofuran-6-ylmethyl)benzamide (32 mg, 0.060 mmol, 1 equiv) in acetonitrile (1.2 mL), water (0.4 mL) and 2,2,2-trifluoroacetic acid (14 mg, 0.12 mmol, 2 equiv) was added citric acid (11 mg, 0.060 mmol, 1 equiv), sodium periodate (51 mg, 0.24 mmol, 4 equiv) and ruthenium (III) chloride (0.6 mg, 3 umol, 0.05 equiv). The resulting mixture was stirred at room temperature for 2.5 h. The mixture was concentrated in vacuo and purified by preparative HPLC on a XBridge Prep OBD C18 Column using a 30-60% gradient of acetonitrile in water to afford 4-[2-(2-aminopyridin-3-yl)-5-phenylimidazo[4,5-b]pyridin-3-yl]-N-[(3-formyl-4-hydroxyphenyl)methyl]benzamide (Example 29) (0.5 mg, 1.5%) as a yellow solid. MS (ESI) calculated for C₃₂H₂₄N₆O₃: 540.19 m/z, found 541.15 [M+H]⁺. ¹H NMR (400 MHz, DMSO-d₆) δ (ppm): 6 10.26 (s, 1H), 8.30-8.36 (m, 1H), 7.99-8.13 (m, 7H), 7.59-7.70 (m, 4H), 7.38-7.57 (m, 5H), 6.97-7.02 (m, 1H), 6.69-6.74 (m, 1H), 4.44 (s, 2H).

### Intermediate 29-2: 4-(2-(2-aminopyridin-3-yl)-5-phenyl-3H-imidazo[4,5-b]pyridin-3-yl) benzoic acid

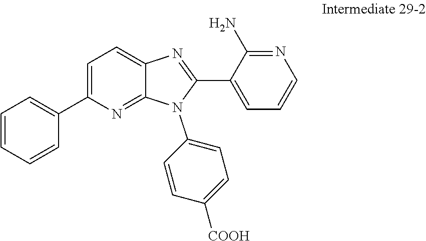 [SMILES: Nc1ncccc1-c1nc2ccc(-c3ccccc3)nc2n1-c1ccc(C(=O)O)cc1]

### Synthetic Route:

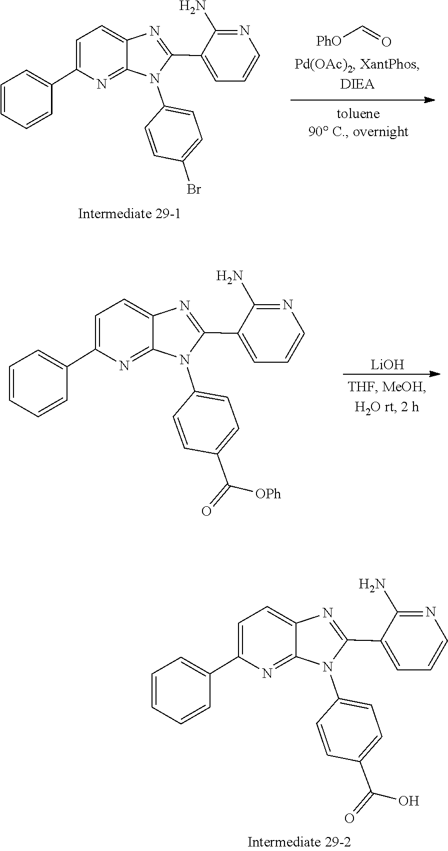 [SMILES: Nc1ncccc1-c1nc2ccc(-c3ccccc3)nc2n1-c1ccc(Br)cc1 ; O=COc1ccccc1 ; Nc1ncccc1-c1nc2ccc(-c3ccccc3)nc2n1-c1ccc(C(=O)Oc2ccccc2)cc1 ; "LiOH" ; Nc1ncccc1-c1nc2ccc(-c3ccccc3)nc2n1-c1ccc(C(=O)O)cc1]

### Step 1: Synthesis of phenyl 4-[2-(2-formamidopyridin-3-yl)-5-phenylimidazo[4,5-b]pyridin-3-yl]benzoate

A solution of 3-(3-(4-bromophenyl)-5-phenyl-3H-imidazo[4,5-b]pyridin-2-yl)pyridin-2-amine (Intermediate 29-1) (500 mg, 1.37 mmol, 1 equiv), phenyl formate (500 mg, 4.10 mmol, 3 equiv), palladium(II) acetate (15 mg, 0.068 mmol, 0.05 equiv), XantPhos (40 mg, 0.068 mmol, 0.05 equiv) and N,N-diisopropylethylamine (353 mg, 2.73 mmol, 2 equiv) in toluene (5 mL) was stirred under nitrogen atmosphere at 90° C. overnight. The resulting mixture was partitioned between ethyl acetate (10 mL) and water (10 mL). The layers were separated, and the aqueous layer was extracted with ethyl acetate (10 mL×2). The combined organic layers were washed with brine (30 mL×3), dried over sodium sulfate, filtered, and concentrated in vacuo to afford phenyl 4-[2-(2-formamidopyridin-3-yl)-5-phenylimidazo[4,5-b]pyridin-3-yl]benzoate (570 mg, 20%) as a brown oil, which was used in the next step without further purification. MS (ESI) calculated for C₃₁H₂₁N₅O₃: 511.16 m/z, found 512.05 [M+H]⁺.

### Step 2: Synthesis of 4-(2-(2-aminopyridin-3-yl)-5-phenyl-3H-imidazo[4,5-b]pyridin-3-yl) benzoic acid (Intermediate 29-2)

To a solution of phenyl 4-[2-(2-formamidopyridin-3-yl)-5-phenylimidazo[4,5-b]pyridin-3-yl]benzoate (570 mg, 1.11 mmol, 1 equiv) in methanol (1 mL), water (1 mL) and tetrahydrofuran (5 mL) was added lithium hydroxide (80 mg, 3.3 mmol, 3 equiv) and the resulting mixture was stirred at room temperature for 2 h. The mixture was concentrated in vacuo and water (2 mL) was added. The pH of the mixture was brought to 3˜4 with 2N hydrochloric acid and extracted with chloroform/isopropanol (3:1, 5 mL×3). The combined organic phases were concentrated in vacuo and the residue was recrystallized from diethyl ether (5 mL) to afford 4-[2-(2-aminopyridin-3-yl)-5-phenylimidazo[4,5-b]pyridin-3-yl]benzoic acid (Intermediate 29-2) (85 mg, 12%) as a yellow solid. MS (ESI) calculated for C₂₄H₁₇N₅O₂: 407.14 m/z, found 408.15 [M+H]⁺.

### Intermediate 29-1: 3-[3-(4-bromophenyl)-5-phenylimidazo[4,5-b]pyridin-2-yl]pyridin-2-amine

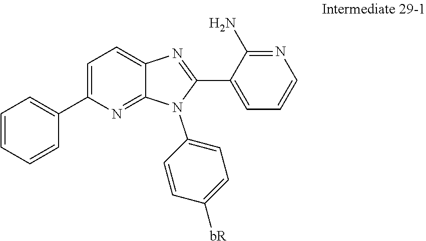

Intermediate 29-1 was prepared in a manner analogous to Intermediate 11-1 (Steps 1 and 2 only) using 4-bromoaniline in place of tert-butyl 2-(4-aminophenyl)acetate. MS (ESI) calculated for C₂₃H₁₆BrN₅: 441.06 m/z, found 442.00 [M+H]⁺.

### Intermediate 29-3: benzofuran-5-ylmethanamine

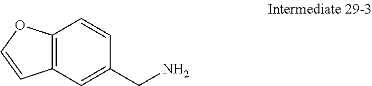 [SMILES: NCc1ccc2occc2c1]

### Synthetic Route:

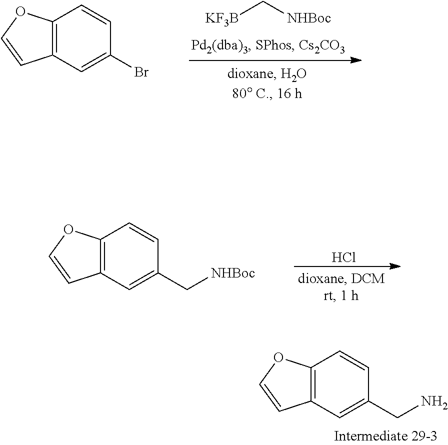

### Step 1: Synthesis of tert-butyl (benzofuran-5-ylmethyl)carbamate

A solution of 5-bromo-1-benzofuran (1.00 g, 5.08 mmol, 1 equiv), tert-butyl ((trifluoro-λ⁴-boraneyl)methyl)carbamate, potassium salt (1.80 g, 7.61 mmol, 1.5 equiv), tris(dibenzylideneacetone)dipalladium(0) (465 mg, 0.508 mmol, 0.1 equiv), SPhos (208 mg, 0.508 mmol, 0.1 equiv) and cesium carbonate (3.30 g, 10.2 mmol, 2 equiv) in 1,4-dioxane (40 mL) and water (10 mL) was stirred at 80° C. for 16 h. The resulting mixture was cooled to room temperature and quenched with water (10 mL). The mixture was extracted with ethyl acetate (500 mL×2) and the combined organic phases were washed with brine (30 mL×3), dried with sodium sulfate, filtered, and concentrated in vacuo. The residue was purified by silica gel column chromatography using a 0-10% gradient of ethyl acetate in petroleum ether to afford tert-butyl N-(1-benzofuran-5-ylmethyl)carbamate (800 mg, 55%) as a yellow oil. MS (ESI) calculated for C₁₄H₁₇NO₃: 247.12 m/z, found 192.00 [M−C(CH₃)₃+H]⁺.

### Step 2: Synthesis of benzofuran-5-ylmethanamine (Intermediate 29-3)

To a solution of tert-butyl N-(1-benzofuran-5-ylmethyl)carbamate (800 mg, 3.24 mmol, 1 equiv) in dichloromethane (5 mL) was added 4N hydrochloric acid in 1,4-dioxane (5 mL) and the resulting mixture was stirred at room temperature for 1 h. The mixture was concentrated in vacuo to provide benzofuran-5-ylmethanamine (Intermediate 29-3) (600 mg, 67%) as a white solid. MS (ESI) calculated for C₉H₉NO: 147.07 m/z, found 148.10 [M+H]⁺.

### Example 30: N-(1-(4-(2-(2-aminopyridin-3-yl)-5-phenyl-3H-imidazo[4,5-b]pyridin-3-yl)phenyl)-2-hydroxyethyl)-2-(2-fluoro-4-formyl-3-hydroxyphenyl)acetamide

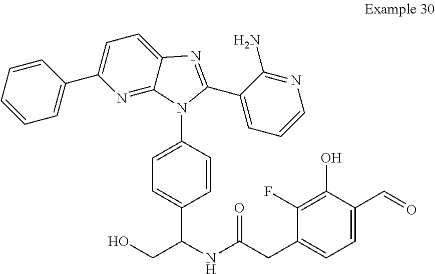 [SMILES: Nc1ncccc1-c1nc2ccc(-c3ccccc3)nc2n1-c1ccc(C(CO)NC(=O)Cc2ccc(C=O)c(O)c2F)cc1]

Example 30 was prepared in a manner analogous to Example 1 (starting from Step 3) using Intermediate 30-2 in place of the amine starting material and Intermediate 21-2 in place of Intermediate 1-4. MS (ESI) calculated for C₃₄H₂₇FN₆O₄: 602.21 m/z, found 603.30 [M+H]⁺. ¹H NMR (400 MHz, DMSO-d₆) δ 10.22 (s, 1H), 8.64-8.74 (m, 1H), 8.23-8.34 (m, 1H), 7.93-8.10 (m, 4H), 7.36-7.55 (m, 6H), 7.13-7.21 (m, 1H), 6.96-7.02 (s, 1H), 6.85-6.95 (m, 1H), 6.32-6.44 (m, 2H), 4.94-5.08 (m, 1H), 3.56-3.77 (m, 4H). ¹⁹F NMR (282 MHz, DMSO-d₆) δ (ppm): −130.09.

### Intermediate 30-2: 2-amino-2-(4-(2-(2-aminopyridin-3-yl)-5-phenyl-3H-imidazo[4,5-b]pyridin-3-yl)phenyl)ethan-1-ol

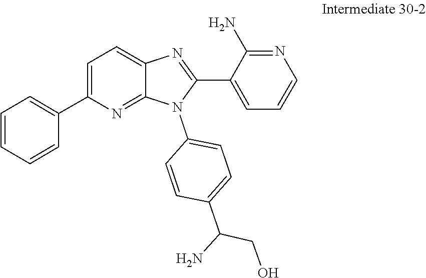 [SMILES: Nc1ncccc1-c1nc2ccc(-c3ccccc3)nc2n1-c1ccc(C(N)CO)cc1]

Intermediate 30-2 was prepared in a manner analogous to Intermediate 11-1 using Intermediate 30-1 in place of tert-butyl 2-(4-aminophenyl)acetate. MS (ESI) calculated for C₂₅H₂₂N₆O: 422.19 m/z, found 423.20 [M+H]⁺.

### Intermediate 30-2: tert-butyl (1-(4-aminophenyl)-2-hydroxyethyl)carbamate

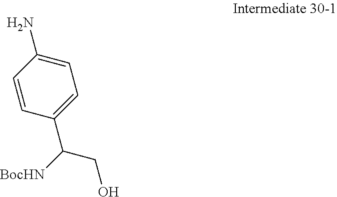 [SMILES: CC(C)(C)OC(=O)NC(CO)c1ccc(N)cc1]

### Synthetic Route:

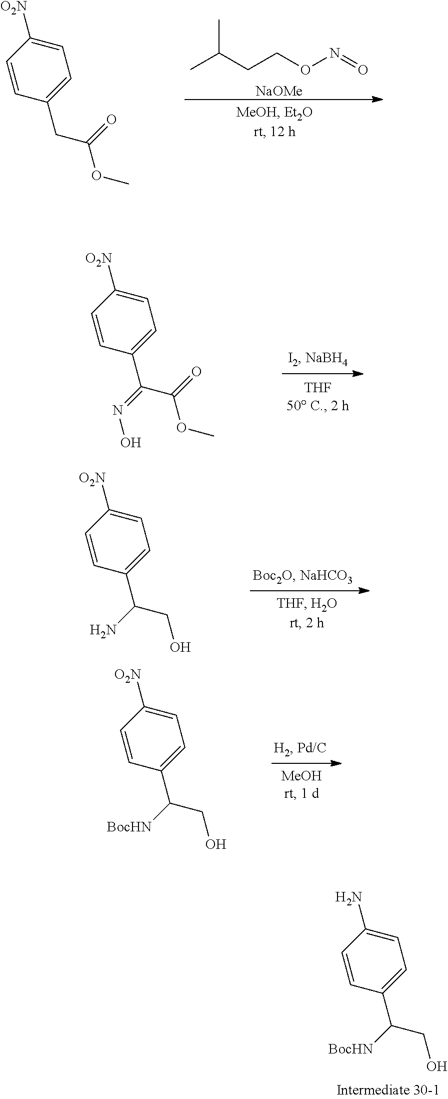

### Step 1: Synthesis of methyl (E)-2-(hydroxyimino)-2-(4-nitrophenyl)acetate

A solution of methyl 2-(4-nitrophenyl)acetate (5.00 g, 25.6 mmol, 1 equiv) in diethyl ether (50 mL) was treated with a solution of isopentyl nitrite (6.60 g, 56.4 mmol, 2.2 equiv) in diethyl ether (35 mL) at room temperature under nitrogen atmosphere followed by dropwise addition of a suspension of sodium methoxide (2.21 g, 41.0 mmol, 1.6 equiv) in methanol (35 mL). The mixture was stirred for 12 h at room temperature. The residue was concentrated and added to 2 N hydrochloric acid followed by stirring at room temperature for 30 min. The solid was filtered and washed with methanol to provide methyl (2E)-2-(N-hydroxyimino)-2-(4-nitrophenyl)acetate (3.1 g, 54%) a white solid. MS (ESI) calculated for C₉H₈N₂O₅: 224.17 m/z, found 223.00 [M−H]⁻.

### Step 2: Synthesis of 2-amino-2-(4-nitrophenyl)ethan-1-ol

To a cooled (0° C.) solution of methyl (2E)-2-(N-hydroxyimino)-2-(4-nitrophenyl)acetate (2.9 g, 13 mmol, 1 equiv) in tetrahydrofuran (50 mL) under nitrogen atmosphere was added sodium borohydride (1.47 g, 38.9 mmol, 3 equiv) followed by a solution of iodine (5.00 g, 19.7 mmol, 1.5 equiv) in tetrahydrofuran (33 mL) dropwise. The reaction mixture was stirred at 50° C. for 2 h, then cooled to 0° C. and quenched with water. Stirring was continued for 30 min and the solution was alkalized to pH 9 with 1N aqueous sodium hydroxide. The resulting mixture was used directly in the next step. MS (ESI) calculated for C₈H₁₀N₂O₃: 182.07 m/z, found 183.10 [M+H]⁺.

### Step 3: Synthesis of tert-butyl (2-hydroxy-1-(4-nitrophenyl)ethyl)carbamate

To the reaction mixture from the previous step was added di-tert-butyl dicarbonate (12.5 mL, 58.4 mmol, 4.5 equiv) and the mixture was stirred at room temperature for 2 h. The resulting mixture was extracted with ethyl acetate (3×50 mL) and the combined organic layers were dried over anhydrous sodium sulfate, filtered, and concentrated under reduced pressure to provide tert-butyl N-[2-hydroxy-1-(4-nitrophenyl)ethyl]carbamate (2.5 g, 81%) as a yellow oil, which was used without purification in the next step. MS (ESI) calculated for C₁₃H₁₈N₂O₅: 282.12 m/z, found 183.10 [M−Boc+H]⁺.

### Step 4: Synthesis of tert-butyl (1-(4-aminophenyl)-2-hydroxyethyl)carbamate (Intermediate 30-1)

A mixture of tert-butyl N-[2-hydroxy-1-(4-nitrophenyl)ethyl]carbamate (2.70 g, 9.56 mmol, 1 equiv) and 10% palladium on carbon (2.70 g, 25.3 mmol, 2.65 equiv) was stirred for 1 day at room temperature under hydrogen atmosphere. The mixture was filtered and concentrated in vacuo to provide tert-butyl N-[1-(4-aminophenyl)-2-hydroxyethyl]carbamate (Intermediate 30-1) (2 g, 83%) as a white oil, which was used in subsequent transformations directly without further purification. MS (ESI) calculated for C₁₃H₂₀N₂O₃: 252.15 m/z, found 253.05 [M+H]⁺.

### Example 31: N-(1-{4-[2-(2-aminopyridin-3-yl)-5-phenylimidazo[4,5-b]pyridin-3-yl]phenyl}cyclopropyl)-2-(2-fluoro-4-formyl-3-hydroxyphenyl)acetamide

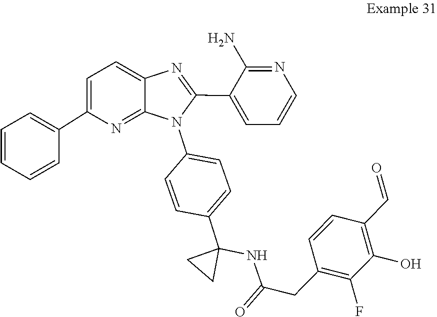 [SMILES: Nc1ncccc1-c1nc2ccc(-c3ccccc3)nc2n1-c1ccc(C2(NC(=O)Cc3ccc(C=O)c(O)c3F)CC2)cc1]

Example 31 was prepared in a manner analogous to Example 1 (starting from Step 3) using Intermediate 31-2 in place of the amine starting material and Intermediate 21-2 in place of Intermediate 1-4. MS (ESI) calculated for C₃₅H₂₇FN₆O₃: 598.21 m/z, found 599.25 [M+H]⁺. ¹H-NMR (300 MHz, DMSO-d₆) δ (ppm): 10.19 (s, 1H), 8.25-8.28 (m, 1H), 7.96-8.03 (m, 4H), 7.37-7.50 (m, 2H), 7.25-7.28 (m, 4H), 7.18-7.22 (m, 3H), 6.75-6.81 (m, 1H), 6.40-6.45 (m, 1H), 3.61 (s, 2H), 1.30-1.33 (m, 2H), 1.23-1.26 (m, 2H).

### Intermediate 31-2: 3-(3-(4-(1-aminocyclopropyl)phenyl)-5-phenyl-3H-imidazo[4,5-b]pyridin-2-yl)pyridin-2-amine

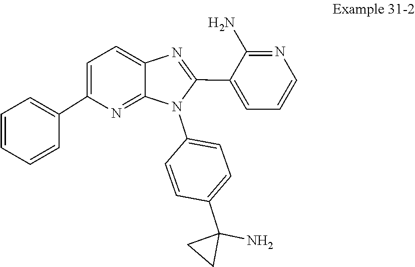 [SMILES: Nc1ncccc1-c1nc2ccc(-c3ccccc3)nc2n1-c1ccc(C2(N)CC2)cc1]

Intermediate 31-2 was prepared in a manner analogous to Intermediate 11-1 using Intermediate 31-1 in place of tert-butyl 2-(4-aminophenyl)acetate. MS (ESI) calculated for C₂₆H₂₂N₆: 418.19 m/z, found 419.25 [M+H]⁺.

### Intermediate 31-1: tert-butyl (1-(4-aminophenyl)cyclopropyl)carbamate

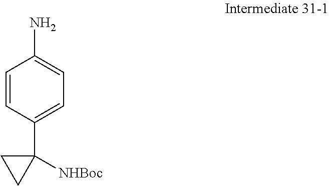 [SMILES: CC(C)(C)OC(=O)NC1(c2ccc(N)cc2)CC1]

### Synthetic Route:

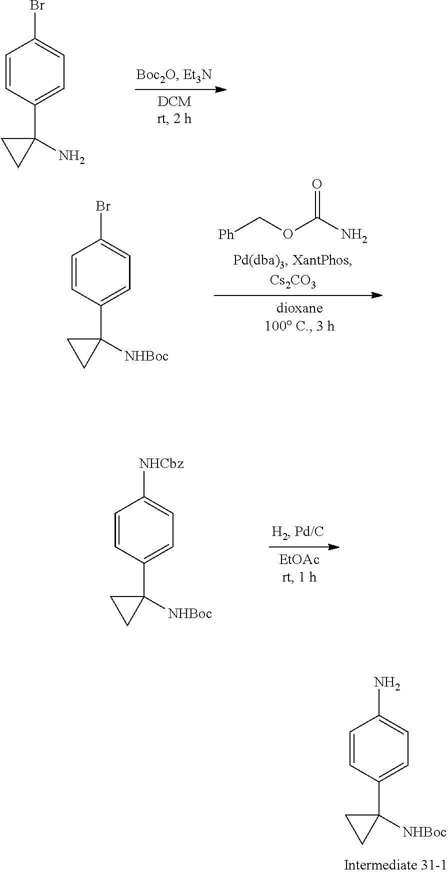 [SMILES: NC1(c2ccc(Br)cc2)CC1 ; CC(C)(C)OC(=O)NC1(c2ccc(Br)cc2)CC1 ; NC(=O)OCc1ccccc1 ; CC(C)(C)OC(=O)NC1(c2ccc(NC(=O)OCc3ccccc3)cc2)CC1 ; CC(C)(C)OC(=O)NC1(c2ccc(N)cc2)CC1]

### Step 1: Synthesis of tert-butyl N-[1-(4-bromophenyl)cyclopropyl]carbamate

To a solution of 1-(4-bromophenyl)cyclopropan-1-amine (5.00 g, 23.6 mmol, 1 equiv) in dichloromethane (50 mL) were added triethylamine (7.16 g, 70.7 mmol, 3 equiv) and di-tert-butyl dicarbonate (10.29 g, 47.15 mmol, 2 equiv). The resulting mixture was stirred at room temperature for 2 h. The reaction was quenched with water (100 mL) and extracted with ethyl acetate (3×100 mL). The organic layers were combined, dried over sodium sulfate, filtered, and concentrated in vacuo. The obtained residue was purified by silica gel column chromatography using a 0-30% gradient of ethyl acetate in petroleum ether to afford tert-butyl N-[1-(4-bromophenyl)cyclopropyl]carbamate (5 g, 68%) as a yellow solid. MS (ESI) calculated for C₁₄H₁₈BrNO₂: 311.05 m/z, found 310.00 [M−H]⁻.

### Step 2: Synthesis of benzyl N-(4-{1-[(tert-butoxycarbonyl)amino]cyclopropyl}phenyl) carbamate

To a solution of tert-butyl N-[1-(4-bromophenyl)cyclopropyl]carbamate (1.8 g, 5.8 mmol, 1 equiv) in 1,4-dioxane (10 mL) were added O-benzyl carbamate (1.05 g, 6.92 mmol, 1.2 equiv), XantPhos (667 mg, 1.15 mmol, 0.2 equiv), tris(dibenzylideneacetone)dipalladium(0) (528 mg, 0.577 mmol, 0.1 equiv) and cesium carbonate (5.64 g, 17.3 mmol, 3 equiv). The resulting mixture was stirred for 3 h at 100° C. under nitrogen atmosphere. The mixture was then cooled to room temperature and quenched with water (100 mL). The resulting mixture was extracted with ethyl acetate (3×100 mL). The organic layers were combined, dried over sodium sulfate, filtered, and concentrated in vacuo. The residue obtained was purified by silica gel column chromatography using a 0-30% gradient of ethyl acetate in petroleum ether to afford benzyl N-(4-{1-[(tert-butoxycarbonyl)amino]cyclopropyl}phenyl)carbamate (1.1 g, 50%) as a yellow solid. MS (ESI) calculated for C₂₂H₂₆N₂O₄: 382.19 m/z, found 381.05 [M−H]⁻.

### Step 3: Synthesis of tert-butyl N-[1-(4-aminophenyl)cyclopropyl]carbamate (Intermediate 31-1)

To a solution of benzyl N-(4-{1-[(tert-butoxycarbonyl)amino]cyclopropyl}phenyl) carbamate (1.1 g, 2.9 mmol, 1 equiv) in methanol (3 mL) was added palladium on carbon (10%, 110 mg, 0.03 equiv) under nitrogen atmosphere. The mixture stirred at room temperature under hydrogen atmosphere for 1 h then filtered and concentrated in vacuo. The residue obtained was purified by silica gel column chromatography using a 0-100% gradient of ethyl acetate in petroleum ether to afford tert-butyl N-[1-(4-aminophenyl)cyclopropyl]carbamate (Intermediate 31-1) (500 mg, 70%) as a yellow solid. MS (ESI) calculated for C₁₄H₂₀N₂O₂: 248.15 m/z, found 247.00 [M−H]⁻.

### Example 32: N-({4-[2-(2-aminopyridin-3-yl)-5-(1-methylpyrazol-4-yl)imidazo[4,5-b]pyridin-3-yl]phenyl}methyl)-2-(4-formyl-3-hydroxyphenyl)acetamide

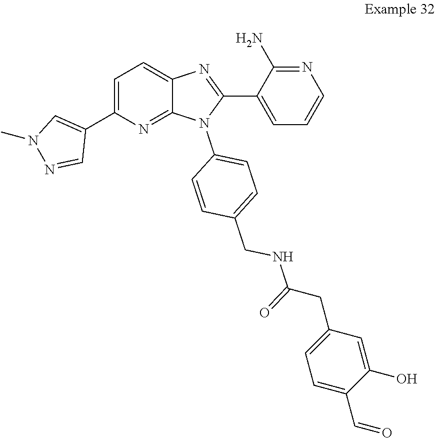 [SMILES: Cn1cc(-c2ccc3nc(-c4cccnc4N)n(-c4ccc(CNC(=O)Cc5ccc(C=O)c(O)c5)cc4)c3n2)cn1]

Example 32 was prepared in a manner analogous to Example 1 using 1-methylpyrazol-4-ylboronic acid in place of 4-(morpholin-4-yl)phenylboronic acid. MS (ESI) calculated for C₃₁H₂₆N₈O₃: 558.21 m/z, found 559.25 [M+H]⁺. ¹H NMR (400 MHz, DMSO-d₆) δ (ppm): 10.17 (s, 1H), 8.12-8.14 (m, 2H), 7.97-7.98 (m, 2H), 7.65-7.67 (m, 2H), 7.36-7.67 (m, 4H), 7.15-7.17 (m, 1H), 6.89-6.94 (m, 2H), 6.37-6.40 (m, 1H), 4.36-4.37 (m, 2H), 3.81-3.88 (m, 3H), 3.48-3.52 (m, 2H).

### Example 33: N-(4-(2-(2-aminopyridin-3-yl)-5-methoxy-3H-imidazo[4,5-b]pyridin-3-yl) benzyl)-2-(4-formyl-3-hydroxyphenyl)acetamide

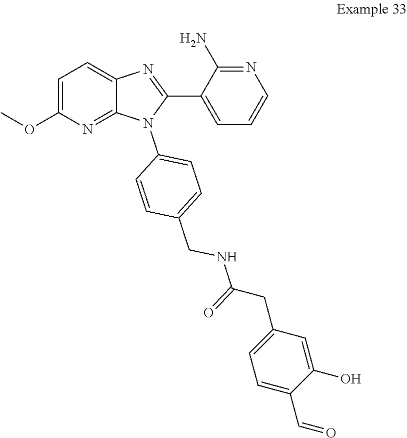 [SMILES: COc1ccc2nc(-c3cccnc3N)n(-c3ccc(CNC(=O)Cc4ccc(C=O)c(O)c4)cc3)c2n1]

Example 33 was prepared in a manner analogous to Example 1 (starting from Step 3) using Intermediate 33-1 in place of the amine starting material. MS (ESI) calculated for C₂₈H₂₄N₆O₄: 508.19 m/z, found 509.20 [M+H]⁺. ¹H NMR (400 MHz, DMSO-d₆) δ 10.14 (s, 1H), 8.08-8.16 (m, 1H), 8.03-7.98 (m, 1H), 7.30-7.54 (m, 2H), 7.30-7.45 (m, 4H), 6.81-7.02 (m, 3H), 6.62-6.80 (m, 1H), 4.30-4.41 (m, 2H), 3.65-3.95 (m, 3H), 3.53-3.67 (m, 2H).

### Intermediate 33-2: 3-(3-(4-(aminomethyl)phenyl)-5-methoxy-3H-imidazo[4,5-b]pyridin-2-yl)pyridin-2-amine

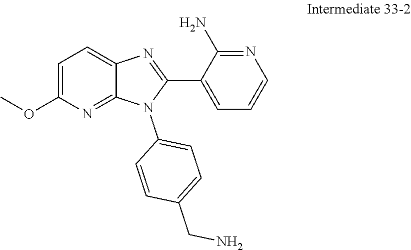 [SMILES: COc1ccc2nc(-c3cccnc3N)n(-c3ccc(CN)cc3)c2n1]

Intermediate 33-2 was prepared in a manner analogous to Intermediate 11-1 using 2-chloro-6-methoxy-3-nitropyridine in place of 2-chloro-3-nitro-6-phenylpyridine and tert-butyl N-[(4-aminophenyl)methyl]carbamate in place of tert-butyl 2-(4-aminophenyl)acetate. MS (ESI) calculated for C₁₉H₁₈N₆O: 346.15 m/z, found 347.39 [M+H]⁺.

### Example 34: N-({4-[2-(2-aminopyridin-3-yl)-5-[2-(morpholin-4-yl)pyridin-4-yl]imidazo[4,5-b]pyridin-3-yl]phenyl}methyl)-2-(4-formyl-3-hydroxyphenyl)acetamide

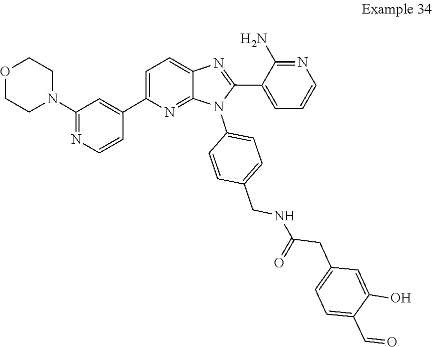 [SMILES: Nc1ncccc1-c1nc2ccc(-c3ccnc(N4CCOCC4)c3)nc2n1-c1ccc(CNC(=O)Cc2ccc(C=O)c(O)c2)cc1]

Example 34 was prepared in a manner analogous to Example 1 using 4-[4-(4,4,5,5-tetramethyl-1,3,2-dioxaborolan-2-yl)pyridin-2-yl]morpholine in place of 4-(morpholin-4-yl)phenylboronic acid. MS (ESI) calculated for C₃₆H₃₂N₈O₄: 640.25 m/z, found 641.30 [M+H]⁺. ¹H NMR (400 MHz, DMSO-d₆) δ (ppm): 10.22 (s, 1H), 8.40-8.50 (m, 1H), 8.25-8.40 (m, 1H), 8.02-8.25 (m, 2H), 7.35-7.80 (m, 8H), 6.75-7.00 (m, 3H), 4.30-4.50 (m, 2H), 3.70-3.82 (m, 2H), 3.60-3.70 (m, 8H).

### Example 35: N-({4-[2-(2-aminopyridin-3-yl)-5-(pyrazol-1-yl)imidazo[4,5-b]pyridin-3-yl]phenyl}methyl)-2-(4-formyl-3-hydroxyphenyl)acetamide

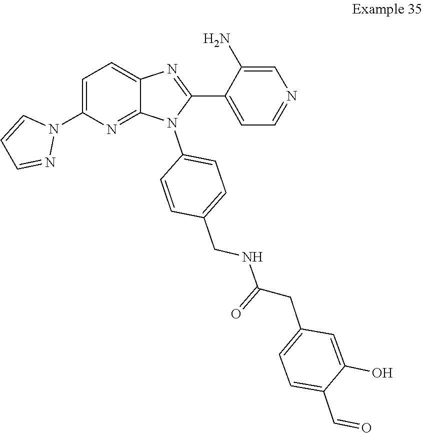 [SMILES: Nc1cnccc1-c1nc2ccc(-n3cccn3)nc2n1-c1ccc(CNC(=O)Cc2ccc(C=O)c(O)c2)cc1]

Example 34 was prepared in a manner analogous to Example 1 (starting from Step 3) using Intermediate 34-1 in place of the amine starting material. MS (ESI) calculated for C₃₀H₂₄N₈O₃: 544.20 m/z, found 545.20 [M+H]⁺. ¹H NMR (400 MHz, DMSO-d₆) δ (ppm): 10.19 (s, 1H), 8.41-8.50 (m, 1H), 8.32-8.39 (m, 1H), 7.98-8.12 (m, 2H), 7.81 (s, 1H), 7.60-7.72 (m, 2H), 7.49-7.51 (m, 2H), 7.39-7.42 (m, 2H), 6.85-7.01 (m, 2H), 6.69-6.81 (m, 1H), 6.55 (s, 1H), 4.49 (s, 2H), 3.51-3.61 (m, 2H).

### Intermediate 35-1: 3-(3-(4-(aminomethyl)phenyl)-5-(1H-pyrazol-1-yl)-3H-imidazo[4,5-b]pyridin-2-yl)pyridin-2-amine

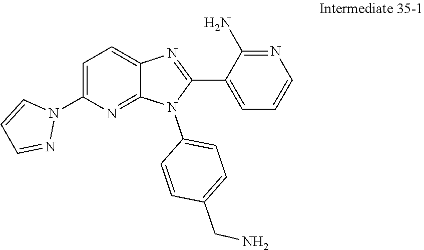 [SMILES: NCc1ccc(-n2c(-c3cccnc3N)nc3ccc(-n4cccn4)nc32)cc1]

### Synthetic Route:

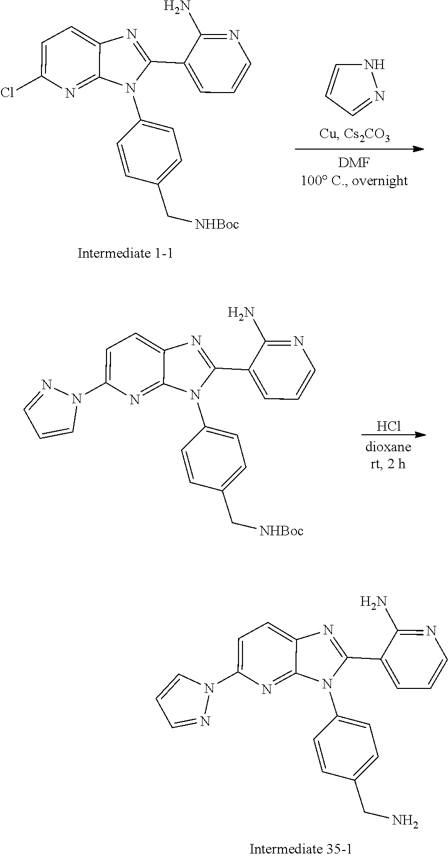 [SMILES: CC(C)(C)OC(=O)NCc1ccc(-n2c(-c3cccnc3N)nc3ccc(Cl)nc32)cc1 ; c1cn[nH]c1 ; CC(C)(C)OC(=O)NCc1ccc(-n2c(-c3cccnc3N)nc3ccc(-n4cccn4)nc32)cc1 ; Cl ; NCc1ccc(-n2c(-c3cccnc3N)nc3ccc(-n4cccn4)nc32)cc1]

### Step 1: Synthesis of tert-butyl N-({4-[2-(2-aminopyridin-3-yl)-5-(pyrazol-1-yl)imidazo[4,5-b]pyridin-3-yl]phenyl}methyl)carbamate

To a solution of tert-butyl N-({4-[2-(2-aminopyridin-3-yl)-5-chloroimidazo[4,5-b]pyridin-3-yl]phenyl}methyl)carbamate (Intermediate 1-1) (500 mg, 1.11 mmol, 1 equiv) and pyrazole (151 mg, 2.22 mmol, 2 equiv) in N,N-dimethylformamide (10 mL) were added copper (7.0 mg, 0.11 mmol, 0.1 equiv) and cesium carbonate (1.08 g, 3.33 mmol, 3 equiv). After stirring overnight at 100° C. under a nitrogen atmosphere, the resulting mixture was poured into water giving a suspension. The solid was filtered, dried under air, and purified by silica gel column chromatography using a 0-20% gradient of ethyl acetate in petroleum ether to afford tert-butyl N-({4-[2-(2-aminopyridin-3-yl)-5-(pyrazol-1-yl)imidazo[4,5-b]pyridin-3-yl]phenyl}methyl)carbamate (230 mg, 41%) as a yellow solid. MS (ESI) calculated for C₂₆H₂₆N₈O₂: 482.22 m/z, found 483.57 [M+H]⁺.

### Step 2: Synthesis of 3-{3-[4-(aminomethyl)phenyl]-5-(pyrazol-1-yl)imidazo[4,5-b]pyridin-2-yl}pyridin-2-amine (Intermediate 35-1)

tert-butyl N-({4-[2-(2-aminopyridin-3-yl)-5-(pyrazol-1-yl)imidazo[4,5-b]pyridin-3-yl]phenyl}methyl)carbamate (230 mg, 0.477 mmol, 1 equiv) was dissolved in 4N hydrochloric acid in 1,4-dioxane and the mixture was stirred at room temperature for 1 h. The mixture was concentrated in vacuo to afford 3-{3-[4-(aminomethyl)phenyl]-5-(pyrazol-1-yl)imidazo[4,5-b]pyridin-2-yl}pyridin-2-amine (Intermediate 35-1) (163 mg, 74%) as a yellow solid, which was used without purification in subsequent transformations. MS (ESI) calculated for C₂₁H₁₈N₈: 382.17 m/z, found 383.20 [M+H]⁺.

### Example 36: 4-(2-((4-(2-(2-aminopyridin-3-yl)-5-phenyl-3H-imidazo[4,5-b]pyridin-3-yl)phenethyl)amino)ethyl)-2-hydroxybenzaldehyde

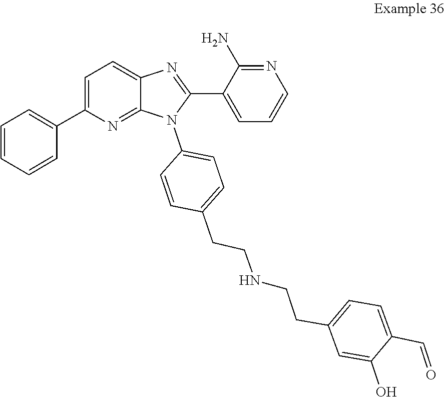 [SMILES: Nc1ncccc1-c1nc2ccc(-c3ccccc3)nc2n1-c1ccc(CCNCCc2ccc(C=O)c(O)c2)cc1]

Example 36 was prepared in a manner analogous to Example 16 using Intermediate 36-1 in place of Intermediate 16-2. MS (ESI) calculated for C₃₄H₃₀N₆O₂, 554.24 m/z, found 555.25 [M+H]⁺. ¹H NMR (400 MHz, DMSO-d₆) δ (ppm): 10.12 (s, 1H), 8.25-8.27 (m, 1H), 7.97-8.02 (m, 4H), 7.56-7.58 (m, 1H), 7.39-7.58 (m, 7H), 7.18-7.36 (m, 1H), 6.83-6.85 (m, 2H), 6.37-6.40 (m, 1H), 2.80-2.85 (m, 8H).

### Intermediate 36-1: 3-(3-(4-(2-aminoethyl)phenyl)-5-phenyl-3H-imidazo[4,5-b]pyridin-2-yl)pyridin-2-amine

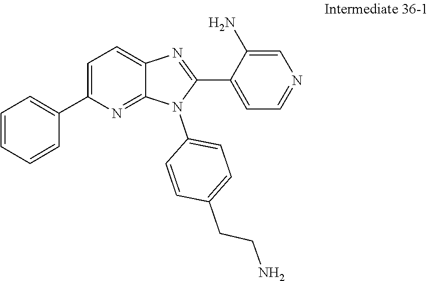 [SMILES: NCCc1ccc(-n2c(-c3ccncc3N)nc3ccc(-c4ccccc4)nc32)cc1]

Intermediate 36-1 was prepared in a manner analogous to Intermediate 11-1 using tert-butyl N-[2-(4-aminophenyl)ethyl]carbamate in place of tert-butyl 2-(4-aminophenyl)acetate. MS (ESI) calculated for C₂₅H₂₂N₆: 406.19 m/z, found 407.05 [M+H]⁺.

### Example 37: N-({4-[2-(2-aminopyridin-3-yl)-5-phenylimidazo[4,5-b]pyridin-3-yl]phenyl}methyl)-2-(4-formyl-3-hydroxyphenyl)propenamide

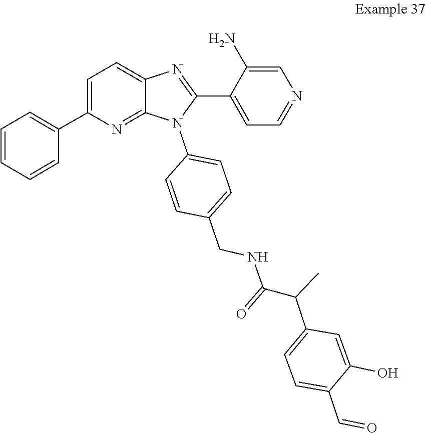 [SMILES: CC(C(=O)NCc1ccc(-n2c(-c3ccncc3N)nc3ccc(-c4ccccc4)nc32)cc1)c1ccc(C=O)c(O)c1]

Example 37 was prepared in a manner analogous to Example 1 (starting from Step 3) using Intermediate 4-1 in place of the amine starting material and Intermediate 37-1 in place of Intermediate 1-4. MS (ESI) calculated for C₃₄H₂₈N₆O₃: 568.22 m/z, found 569.25 [M+H]⁺. ¹H NMR (300 MHz, DMSO-d₆) δ (ppm): 10.72 (s, 1H), 10.20 (s, 1H), 8.66-8.70 (m, 1H), 8.25-8.30 (m, 1H), 7.95-8.05 (m, 4H), 7.60-7.65 (m, 1H), 7.30-7.50 (m, 7H), 7.15-7.25 (m, 1H), 6.94-7.05 (m, 4H), 6.37-6.42 (m, 1H), 4.35-4.40 (m, 2H), 3.70-3.75 (m, 1H), 1.37-1.40 (m, 3H).

### Intermediate 37-1: 2-(4-(1,3-dioxolan-2-yl)-3-((4-methoxybenzyl)oxy)phenyl)propanoic acid

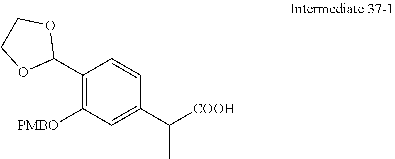

### Synthetic Route:

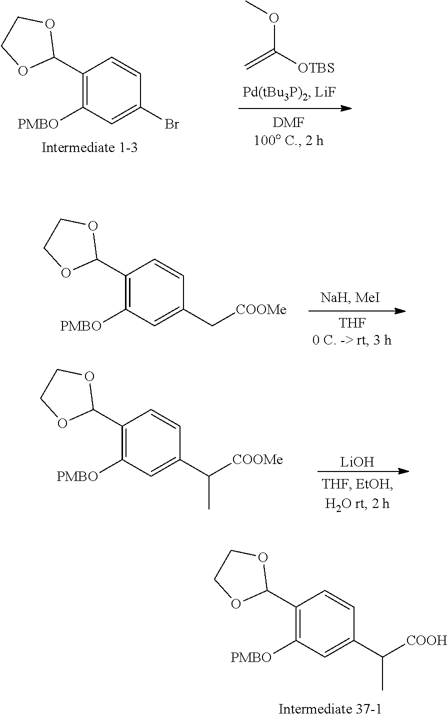

### Step 1: Synthesis of methyl 2-(4-(1,3-dioxolan-2-yl)-3-((4-methoxybenzyl)oxy)phenyl) acetate

To a solution of 2-(4-bromo-2-((4-methoxybenzyl)oxy)phenyl)-1,3-dioxolane (Intermediate 1-3) (4.15 g, 11.3 mmol, 1 equiv) and tert-butyldimethylsilyl methyl carbonate (8.65 g, 45.4 mmol, 4 equiv) in N,N-dimethylformamide (20 mL) were added bis(tri-tert-butylphosphine)palladium(0) (0.60 g, 1.14 mmol, 0.1 equiv) and lithium fluoride (0.6 g, 23 mmol, 2 equiv). The resulting mixture was stirred at 100° C. under nitrogen atmosphere for 2 h then cooled to 0° C. and quenched with water (100 mL). The resulting mixture was extracted with ethyl acetate (200 mL×3). The combined organic layers were washed with water (100 mL×3), dried over anhydrous sodium sulfate, filtered, and concentrated in vacuo. The residue was purified by silica gel column chromatography using a 0-60% gradient of ethyl acetate in petroleum ether to provide methyl 2-(4-(1,3-dioxolan-2-yl)-3-((4-methoxybenzyl)oxy)phenyl)acetate (3.0 g, 74%) as a light-yellow oil. MS (ESI) calculated for C₂₀H₂₂O₆: 358.14 m/z, found 359.10 [M+H]⁺.

### Step 2: Synthesis of methyl 2-[4-(1,3-dioxolan-2-yl)-3-[(4-methoxyphenyl)methoxy]phenyl]propanoate

To a cooled (0° C.) solution of methyl 2-[4-(1,3-dioxolan-2-yl)-3-[(4-methoxyphenyl)methoxy]phenyl]acetate (500 mg, 1.40 mmol, 1 equiv) in tetrahydrofuran (5 mL) was added sodium hydride (50 mg, 2.1 mmol, 1.5 equiv) and the resulting mixture was stirred at 0° C. for 30 minutes. Methyl iodide (297 mg, 2.09 mmol, 1.5 equiv) was added dropwise at 0° C. and the resulting mixture was warmed to room temperature and stirred for 2 h. The reaction was quenched by addition of 1M hydrochloric acid until the pH was between 5 and 6. The mixture was extracted with ethyl acetate and the combined organic extracts were washed with brine, dried over anhydrous sodium sulfate, filtered, and concentrated in vacuo. The resulting residue was purified by silica gel column chromatography using a 0-30% gradient of ethyl acetate in petroleum ether to afford methyl 2-[4-(1,3-dioxolan-2-yl)-3-[(4-methoxyphenyl)methoxy]phenyl]propanoate as a yellow oil. MS (ESI) calculated for C₂₁H₂₄O₆: 372.16 m/z, found 373.10 [M+H]⁺.

### Step 3: Synthesis of 2-[4-(1,3-dioxolan-2-yl)-3-[(4-methoxyphenyl)methoxy]phenyl]propanoic acid (Intermediate 37-1)

To a stirred solution of methyl 2-[4-(1,3-dioxolan-2-yl)-3-[(4-methoxyphenyl)methoxy]phenyl]propanoate (120 mg, 0.322 mmol, 1 equiv) in tetrahydrofuran (3 mL) and methanol (3 mL) was added a solution of lithium hydroxide (15 mg, 0.64 mmol, 2 equiv) in water (3 mL). The resulting mixture was stirred for 2 h at room temperature. The mixture was acidified to pH 5-6 with 1M hydrochloric acid. The resulting mixture was extracted with ethyl acetate (3×50 mL) and the combined organic layers were washed with brine (50 mL), dried over anhydrous sodium sulfate, filtered and concentrated in vacuo to provide 2-[4-(1,3-dioxolan-2-yl)-3-[(4-methoxyphenyl)methoxy]phenyl]propanoic acid (Intermediate 37-1) (100 mg, 87%) as a yellow oil, which was used in subsequent transformations without further purification. MS (ESI) calculated for C₂₀H₂₂O₆: 358.14 m/z, found 359.15 [M+H]⁺.

### Example 38: 4-(((1-(4-(2-(2-aminopyridin-3-yl)-5-phenyl-3H-imidazo[4,5-b]pyridin-3-yl)phenyl)azetidin-3-yl)amino)methyl)-2-hydroxybenzaldehyde

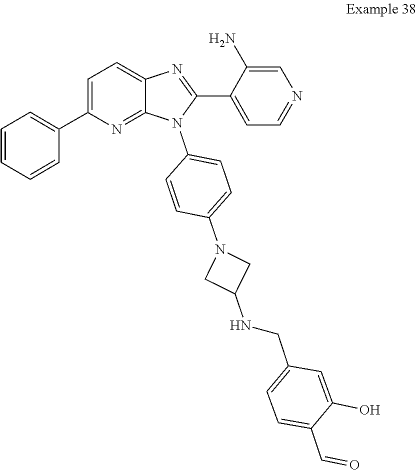 [SMILES: Nc1cnccc1-c1nc2ccc(-c3ccccc3)nc2n1-c1ccc(N2CC(NCc3ccc(C=O)c(O)c3)C2)cc1]

Example 38 was prepared in a manner analogous to Example 19 using Intermediate 38-1 in place of Intermediate 19-2. MS (ESI) calculated for C₃₄H₂₉N₇02: 567.20 m/z, found 568.20 [M+H]⁺. ¹H NMR (400 MHz, DMSO-d₆) δ 10.18 (s, 1H), 8.20-8.25 (m, 1H), 7.92-8.03 (m, 4H), 7.60-7.64 (m, 1H), 7.37-7.50 (m, 3H), 7.19-7.28 (m, 3H), 6.94-7.03 (m, 2H), 6.49-6.55 (m, 2H), 6.41-6.45 (m, 1H), 4.02-4.08 (m, 2H), 3.66-3.74 (m, 3H), 3.55-3.60 (m, 2H).

### Intermediate 38-1: 3-(3-(4-(3-aminoazetidin-1-yl)phenyl)-5-phenyl-3H-imidazo[4,5-b]pyridin-2-yl)pyridin-2-amine

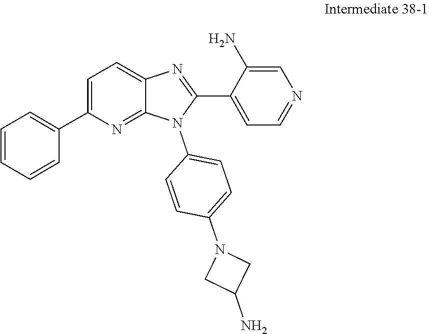 [SMILES: Nc1cnccc1-c1nc2ccc(-c3ccccc3)nc2n1-c1ccc(N2CC(N)C2)cc1]

### Synthetic Route:

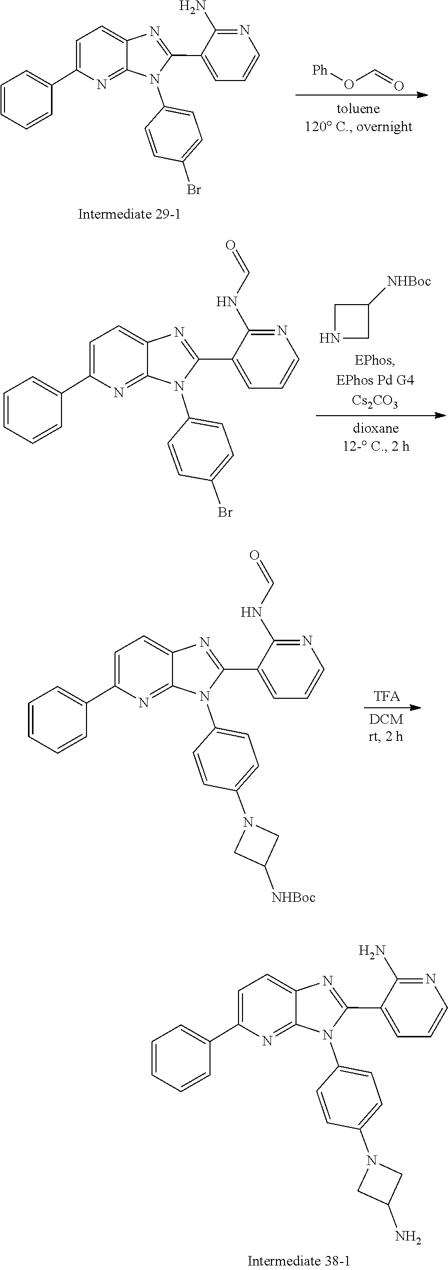

### Step 1: Synthesis of N-(3-(3-(4-bromophenyl)-5-phenyl-3H-imidazo[4,5-b]pyridin-2-yl)pyridin-2-yl)formamide

A suspension of 3-[3-(4-bromophenyl)-5-phenylimidazo[4,5-b]pyridin-2-yl]pyridin-2-amine (Intermediate 29-1) (4.00 g, 9.04 mmol, 1 equiv) and phenyl formate (4.42 g, 36.2 mmol, 4 equiv) in toluene (70 mL) was stirred at 120° C. for 16 h. After concentration, the residue was purified by silica gel column chromatography using a 0-5% gradient of methanol in dichloromethane to afford N-{3-[3-(4-bromophenyl)-5-phenylimidazo[4,5-b]pyridin-2-yl]pyridin-2-yl}formamide (1.9 g, 445%) as a yellow solid. MS (ESI) calculated for C₂₄H₁₆BrN₅O: 469.05 m/z, found 470.05, 472.05 [M+H, M+2+H]⁺.

### Step 2: Synthesis of tert-butyl (1-(4-(2-(2-formamidopyridin-3-yl)-5-phenyl-3H-imidazo[4,5-b]pyridin-3-yl)phenyl)azetidin-3-yl)carbamate

A suspension of N-{3-[3-(4-bromophenyl)-5-phenylimidazo[4,5-b]pyridin-2-yl]pyridin-2-yl}formamide (169 mg, 0.359 mmol, 1 equiv), tert-butyl N-(azetidin-3-yl)carbamate (124 mg, 0.718 mmol, 2 equiv), EPhos (19 mg, 0.036 mmol, 0.1 equiv), EPhos Pd G4 (33 mg, 0.036 mmol, 0.1 equiv) and cesium carbonate (234 mg, 0.718 mmol, 2 equiv) in 1,4-dioxane (5 mL) was stirred at 120° C. for 2 h under nitrogen atmosphere. The reaction mixture was cooled to room temperature and quenched with water (20 mL). The resulting mixture was extracted with ethyl acetate (20 mL×3) and the combined organic phases were washed with brine (30 mL×3), dried with sodium sulfate, filtered, and concentrated in vacuo. The residue was purified by silica gel column chromatography using a 0-85% gradient of ethyl acetate in dichloromethane to afford tert-butyl N-(1-{4-[2-(2-aminopyridin-3-yl)-5-phenylimidazo[4,5-b]pyridin-3-yl]phenyl}azetidin-3-yl)carbamate (156 mg, 62%) as a yellow solid. MS (ESI) calculated for C₃₁H₃₁N₇O₂: 533.25 m/z, found 534.20 [M+H]⁺.

### Step 3: Synthesis of 3-(3-(4-(3-aminoazetidin-1-yl)phenyl)-5-phenyl-3H-imidazo[4,5-b]pyridin-2-yl)pyridin-2-amine (Intermediate 38-1)

A mixture of tert-butyl N-(1-{4-[2-(2-aminopyridin-3-yl)-5-phenylimidazo[4,5-b]pyridin-3-yl]phenyl}azetidin-3-yl)carbamate (150 mg, 0.281 mmol, 1 equiv) in 2,2,2-trifluoroacetic acid (1 mL) and dichloromethane (5 mL) was stirred at room temperature for 2 h. The mixture was concentrated in vacuo and dissolved in dichloromethane (20 mL). The pH of the mixture was brought to -9 with triethylamine. The mixture was concentrated in vacuo and the resulting residue was purified by silica gel column chromatography using a 0-20% gradient of methanol in dichloromethane to afford 3-{3-[4-(3-aminoazetidin-1-yl)phenyl]-5-phenylimidazo[4,5-b]pyridin-2-yl}pyridin-2-amine (130 mg, 98%) as a light-yellow solid. MS (ESI) calculated for C₂₆H₂₃N₇: 433.20 m/z, found 434.15 [M+H]⁺.

### Example 39: 4-{[(2-{4-[2-(2-aminopyridin-3-yl)-5-phenylimidazo[4,5-b]pyridin-3-yl]phenyl}ethyl)(methyl)amino]methyl}-2-hydroxybenzaldehyde

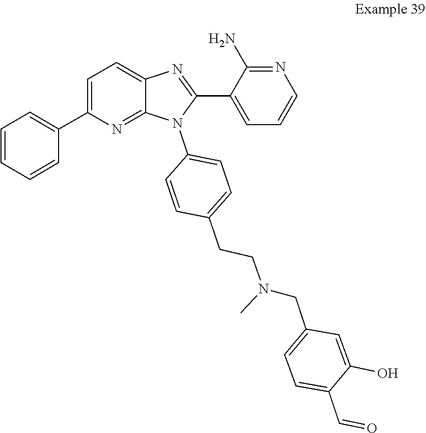 [SMILES: CN(CCc1ccc(-n2c(-c3cccnc3N)nc3ccc(-c4ccccc4)nc32)cc1)Cc1ccc(C=O)c(O)c1]

Example 39 was prepared in a manner analogous to Example 19 using Intermediate 39-2 in place of Intermediate 19-2. MS (ESI) calculated for C₃₄H₃₀N₆O₂: 554.24 m/z, found 555.30 [M+H]⁺. ¹H-NMR (400 MHz, DMSO-d₆) δ (ppm): 10.28 (s, 1H), 8.32-8.34 (m, 1H), 8.02-8.05 (m, 4H), 7.77 (s, 1H), 7.50-7.53 (m, 1H), 7.47-7.49 (m, 7H), 7.15-7.20 (m, 2H), 6.65-6.75 (m, 1H), 4.31-4.49 (m, 2H), 3.62-3.64 (m, 2H), 3.11-3.30 (m, 2H), 2.80 (s, 3H).

### Intermediate 39-2: 3-(3-(4-(2-(methylamino)ethyl)phenyl)-5-phenyl-3H-imidazo[4,5-b]pyridin-2-yl)pyridin-2-amine

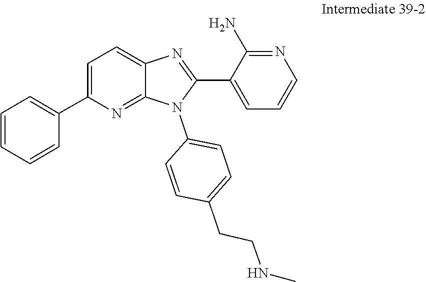 [SMILES: CNCCc1ccc(-n2c(-c3cccnc3N)nc3ccc(-c4ccccc4)nc32)cc1]

Intermediate 39-2 was prepared in a manner analogous to Intermediate 11-1 using Intermediate 39-1 in place of tert-butyl 2-(4-aminophenyl)acetate. MS (ESI) calculated for C₂₆H₂₄N₆: 420.21 m/z, found 421.15 [M+H]⁺.

### Intermediate 39-1: tert-butyl (4-aminophenethyl)(methyl)carbamate

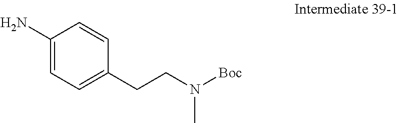 [SMILES: CN(CCc1ccc(N)cc1)C(=O)OC(C)(C)C]

### Synthetic Route:

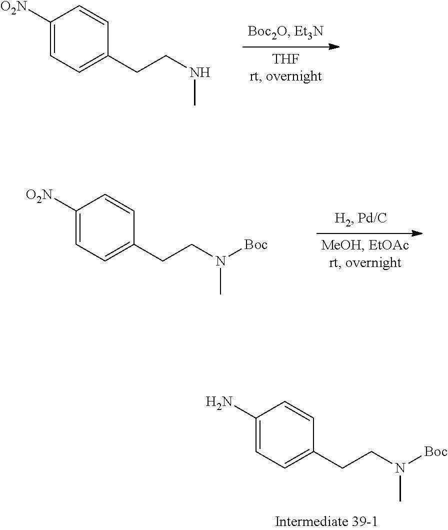 [SMILES: CNCCc1ccc([N+](=O)[O-])cc1 ; CN(CCc1ccc([N+](=O)[O-])cc1)C(=O)OC(C)(C)C ; CN(CCc1ccc(N)cc1)C(=O)OC(C)(C)C]

### Step 1: Synthesis of tert-butyl N-methyl-N-[2-(4-nitrophenyl)ethyl]carbamate

A solution of methyl[2-(4-nitrophenyl)ethyl]amine (5.00 g, 27.7 mmol, 1 equiv), di-tert-butyl dicarbonate (7.56 g, 34.6 mmol, 1.25 equiv) and triethylamine (7.02 g, 69.4 mmol, 2.5 equiv) in dichloromethane (250 mL) was stirred overnight at room temperature. The resulting mixture was concentrated under reduced pressure. The residue was diluted with water and extracted with dichloromethane (50 mL×3). The combined organic layers were washed with water (50 mL×3), dried over anhydrous sodium sulfate, filtered, and concentrated in vacuo to afford tert-butyl N-methyl-N-[2-(4-nitrophenyl)ethyl]carbamate (6 g, 71%) as a yellow oil, which was used in the next step without further purification. MS (ESI) calculated for C₁₄H₂₀N₂O₄: 280.14 m/z, found 281.20 [M+H]⁺.

### Step 2: Synthesis of tert-butyl N-[2-(4-aminophenyl)ethyl]-N-methylcarbamate

A mixture of tert-butyl N-methyl-N-[2-(4-nitrophenyl)ethyl]carbamate (5.00 g, 17.8 mmol, 1 equiv) and 10% palladium on carbon (1.90 g, 17.8 mmol, 1 equiv) in methanol (50 mL) and ethyl acetate (100 mL) was stirred overnight at room temperature under hydrogen atmosphere. The resulting mixture was filtered, and the filter cake was washed with ethyl acetate/methanol (200 mL). The filtrate was concentrated under reduced pressure and the resulting residue was purified by silica gel column chromatography using a 0-100% gradient of ethyl acetate in petroleum ether to afford tert-butyl N-[2-(4-aminophenyl)ethyl]-N-methylcarbamate (Intermediate 39-1) (2.5 g, 60%) as a red oil. MS (ESI) calculated for C₄H₂₂N₂O₂: 250.17 m/z, found 251.30 [M+H]⁺.

### Example 40: 4-(((4-(2-(2-aminopyridin-3-yl)-5-phenyl-3H-imidazo[4,5-b]pyridin-3-yl)phenethyl)amino)methyl)-3-fluoro-2-hydroxybenzaldehyde

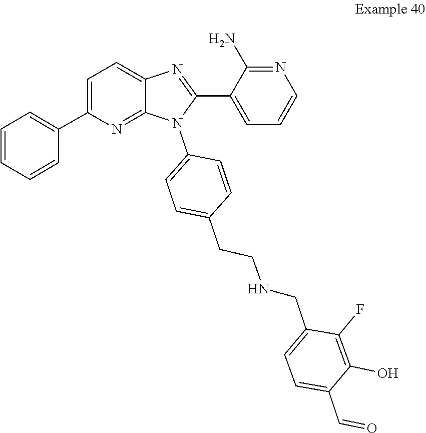 [SMILES: Nc1ncccc1-c1nc2ccc(-c3ccccc3)nc2n1-c1ccc(CCNCc2ccc(C=O)c(O)c2F)cc1]

Example 40 was prepared in a manner analogous to Example 19 using Intermediate 36-1 in place of Intermediate 19-2 and Intermediate 40-1 in place of Intermediate 19-3. MS (ESI) calculated for C₃₂H₂₆FN₇02: 558.21 m/z, found 559.22 [M+H]⁺. ¹H-NMR (400 MHz, DMSO-d₆) δ (ppm): 10.17 (s, 1H), 8.25-8.27 (m, 1H), 7.96-8.02 (m, 4H), 7.37-7.47 (m, 8H), 7.17-7.19 (m, 1H), 6.87-6.94 (m, 1H), 6.37-6.39 (m, 1H), 3.72-3.82 (m, 2H), 2.85 (s, 4H). ¹⁹F-NMR (400 MHz, DMSO-d₆) δ (ppm): −141.32.

### Intermediate 40-1: 4-(1,3-dioxolan-2-yl)-2-fluoro-3-((4-methoxybenzyl)oxy)benzaldehyde

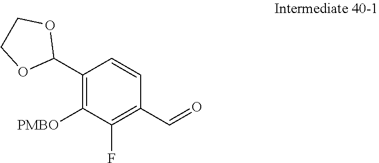

### Synthetic Route:

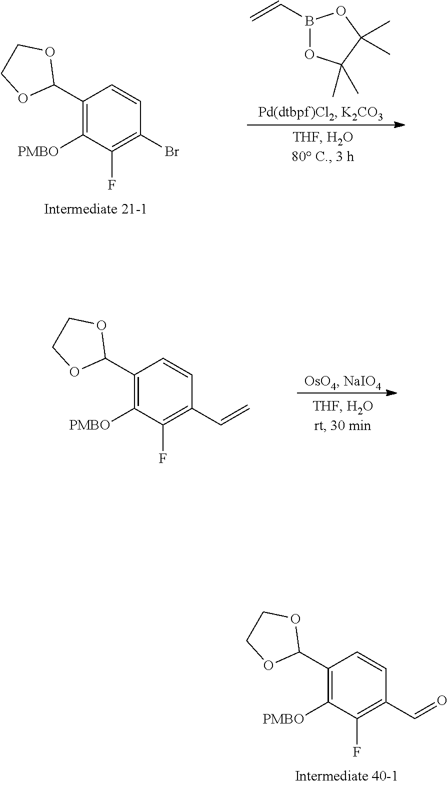

### Step 1: Synthesis of 2-{4-ethenyl-3-fluoro-2-[(4-methoxyphenyl)methoxy]phenyl}-1,3-dioxolane

A mixture of 2-{4-bromo-3-fluoro-2-[(4-methoxyphenyl)methoxy]phenyl}-1,3-dioxolane (Intermediate 21-1) (1.00 g, 2.61 mmol, 1 equiv), 2-ethenyl-4,4,5,5-tetramethyl-1,3,2-dioxaborolane (2.01 g, 13.1 mmol, 5 equiv), [1,1′-bis(di-tert-butylphosphino)ferrocene]dichloropalladium(II) (170 mg, 0.26 mmol, 0.1 equiv) and potassium carbonate (1.08 g, 7.83 mmol, 3 equiv) in tetrahydrofuran (8 mL) and water (2 mL) was stirred for 3 h at 80° C. under nitrogen atmosphere. The mixture cooled to room temperature and quenched with water (100 mL). The mixture was extracted with ethyl acetate (3×100 mL) and the combined organic phases were concentrated under reduced pressure. The residue was purified by reverse-phase flash column chromatography on C18 silica gel using a 10-50% gradient of acetonitrile in water (+0.05% ammonium bicarbonate) to afford 2-{4-ethenyl-3-fluoro-2-[(4-methoxyphenyl)methoxy]phenyl}-1,3-dioxolane (800 mg, 92%) as a yellow oil. MS (ESI) calculated for C₁₉H₁₉FO₄: 330.13 m/z, found 331.13 [M+H]⁺.

### Step 2 Synthesis of 4-(1,3-dioxolan-2-yl)-2-fluoro-3-[(4-methoxyphenyl)methoxy]benzaldehyde (Intermediate 40-1)

A mixture of 2-{4-ethenyl-3-fluoro-2-[(4-methoxyphenyl)methoxy]phenyl}-1,3-dioxolane (260 mg, 0.787 mmol, 1 equiv), osmium tetroxide (0.30 mL, 5.79 mmol, 7.4 equiv) and sodium periodate (505 mg, 2.36 mmol, 3 equiv) in tetrahydrofuran (10 mL) and water (10 mL) was stirred for 20 min at room temperature. The reaction was quenched with water (50 mL) and the mixture was extracted with ethyl acetate (3×40 mL). The combined organic phases were dried with sodium sulfate, filtered, and concentrated in vacuo. The residue was purified by silica gel column chromatography (eluting with petroleum ether/ethyl acetate (10:1)) to afford 4-(1,3-dioxolan-2-yl)-2-fluoro-3-[(4-methoxyphenyl)methoxy]benzaldehyde (Intermediate 40-1) (150 mg, 57%) as a white solid. MS (ESI) calculated for C₁₈H₁₇FO₅: 332.11 m/z, found 333.11 [M+H]⁺.

### Example 41: N-(4-(2-(2-aminopyridin-3-yl)-5-phenyl-3H-imidazo[4,5-b]pyridin-3-yl)benzyl)-3-(tert-butyl)-5-formyl-4-hydroxybenzamide

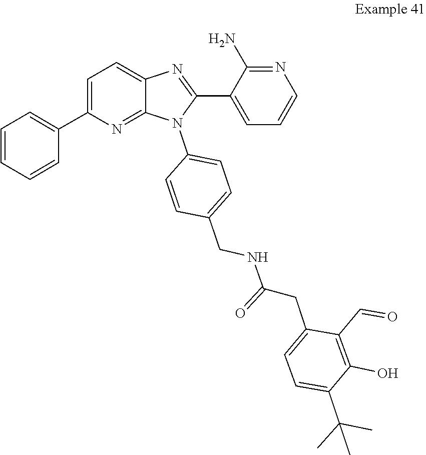 [SMILES: CC(C)(C)c1ccc(CC(=O)NCc2ccc(-n3c(-c4cccnc4N)nc4ccc(-c5ccccc5)nc43)cc2)c(C=O)c1O]

Example 41 was prepared in manner analogous to Example 1 (starting from Step 3) using Intermediate 4-1 in place of the amine starting material and Intermediate 41-1 in place of Intermediate 1-4. MS (ESI) calculated for C₃₆H₃₂N₆O₃: 596.25 m/z, found 597.1 [M+H]⁺. ¹H NMR (300 MHz, DMSO-d₆) δ (ppm) 12.08 (s, 1H), 10.04 (s, 1H), 9.10-9.34 (m, 1H), 8.27-8.36 (m, 2H), 7.90-8.20 (m, 5H), 7.69-7.98 (m, 2H), 7.29-7.51 (m, 7H), 6.64-6.75 (m, 1H), 4.62 (d, J=5.9 Hz, 2H), 1.42 (s, 9H).

### Intermediate 41-1: 3-(tert-butyl)-5-formyl-4-((4-methoxybenzyl)oxy)benzoic acid

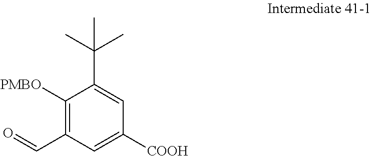

### Synthetic Route:

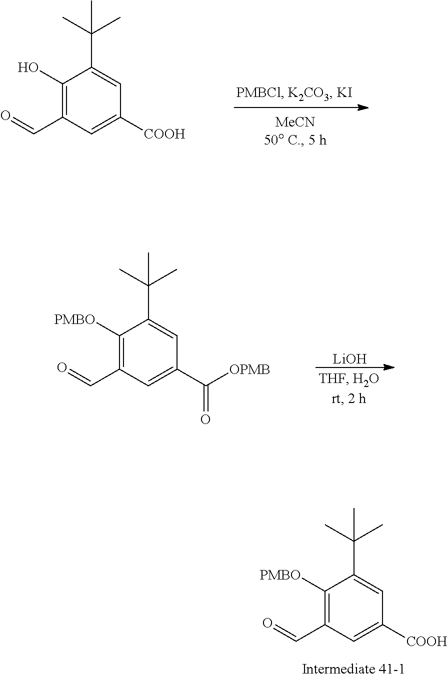

### Step 1: Synthesis of 4-methoxybenzyl 3-(tert-butyl)-5-formyl-4-((4-methoxy benzyl)oxy)benzoate

To a stirred solution of 3-tert-butyl-5-formyl-4-hydroxybenzoic acid (250 mg, 1.13 mmol, 1 equiv), p-methoxybenzyl chloride (210 mg, 1.35 mmol, 1.2 equiv) and potassium carbonate (621 mg, 4.50 mmol, 4 equiv) in anhydrous acetonitrile (5 mL) was added potassium iodide (93 mg, 0.56 mmol, 0.5 equiv) and the resulting mixture was stirred at 50° C. for 5 h. Water (10 mL) was added and the mixture was extracted with ethyl acetate (10 mL×3). The combined organic layers were washed with brine (30 mL×3), dried over anhydrous sodium sulfate, filtered, and concentrated in vacuo. The residue was purified by silica gel column chromatography using a 0-50% gradient of ethyl acetate in petroleum ether to afford (4-methoxyphenyl)methyl 3-tert-butyl-5-formyl-4-[(4-methoxyphenyl)methoxy]benzoate (260 mg, 50%) as a yellow solid. MS (ESI) calculated for C₂₈H₃₀O₆: 462.20 m/z, found 463.20 [M+H]⁺.

### Step 2: Synthesis of 3-tert-butyl-5-formyl-4-[(4-methoxyphenyl)methoxy]benzoic acid (Intermediate 41-1)

To a stirred solution of (4-methoxyphenyl)methyl 3-tert-butyl-5-formyl-4-[(4-methoxyphenyl)methoxy]benzoate (280 mg, 0.605 mmol, 1 equiv) in tetrahydrofuran (5 mL) was added a solution of lithium hydroxide (87 mg, 3.6 mmol, 6 equiv) in water (4 mL) and the resulting mixture was stirred at room temperature for 2 h. The mixture was concentrated in vacuo and purified by reverse-phase column chromatography on C18 silica gel using a 0-95% gradient of acetonitrile in water (+0.05% ammonium bicarbonate) to afford 3-tert-butyl-5-formyl-4-[(4-methoxyphenyl)methoxy]benzoic acid (Intermediate 41-1) (200 mg, 96%) as a yellow solid. MS (ESI) calculated for C₂₀H₂₂O₅: 342.15 m/z, found 341.2 [M+H]⁺.

### Example 42: N-({4-[2-(2-aminopyridin-3-yl)-5-{2-fluoro-3-[4-(2-oxopyrrolidin-1-yl)piperidin-1-yl]phenyl}imidazo[4,5-b]pyridin-3-yl]phenyl}methyl)-2-(4-formyl-3-hydroxyphenyl)acetamide

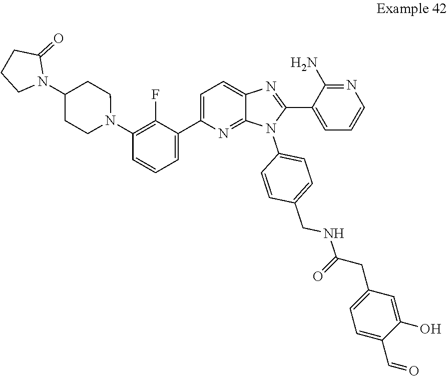 [SMILES: Nc1ncccc1-c1nc2ccc(-c3cccc(N4CCC(N5CCCC5=O)CC4)c3F)nc2n1-c1ccc(CNC(=O)Cc2ccc(C=O)c(O)c2)cc1]

Example 42 was prepared in a manner analogous to Example 1 using Intermediate 42-2 in place of 4-(morpholin-4-yl)phenylboronic acid. MS (ESI) calculated for C₄₂H₃₉FN₈O₄: 738.31 m/z, found 739.25 [M+H]⁺. ¹H NMR (400 MHz, DMSO-d₆) δ (ppm): 10.22 (s, 1H), 8.30-8.35 (m, 1H), 8.00-8.10 (m, 1H), 7.75-7.85 (m, 1H), 7.65-7.75 (m, 1H), 7.55-7.62 (m, 1H), 7.40-7.50 (m, 2H), 7.30-7.40 (m, 2H), 7.20-7.30 (m, 1H), 7.15-7.20 (m, 2H), 6.95 (s, 1H), 6.80-6.95 (m, 1H), 6.70-6.80 (m, 1H), 4.25-4.40 (m, 4H), 3.85-4.00 (m, 1H), 3.55 (s, 2H), 3.30-3.40 (m, 2H), 2.70-2.90 (m, 2H), 2.20-2.30 (m, 2H), 1.80-2.00 (m, 4H), 1.60-1.70 (m, 2H).

### Intermediate 42-2: (2-fluoro-3-(4-(2-oxopyrrolidin-1-yl)piperidin-1-yl)phenyl)boronic acid

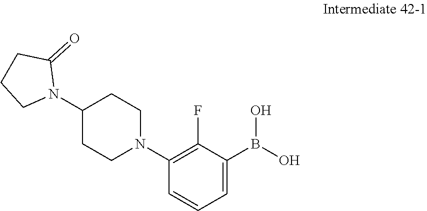 [SMILES: O=C1CCCN1C1CCN(c2cccc(B(O)O)c2F)CC1]

### Synthetic Route:

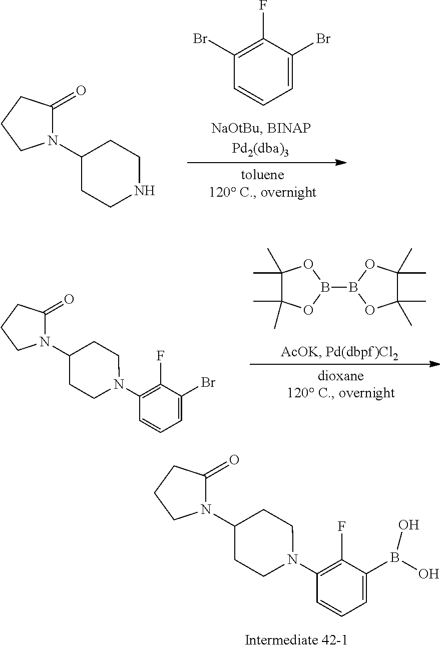

### Step 1: Synthesis of 1-[1-(3-bromo-2-fluorophenyl)piperidin-4-yl]pyrrolidin-2-one

To a mixture of 1-(piperidin-4-yl)pyrrolidin-2-one (1.00 g, 5.94 mmol, 1 equiv), 1,3-dibromo-2-fluorobenzene (1.51 g, 5.94 mmol, 1 equiv) and sodium tert-butoxide (1.71 g, 17.8 mmol, 3 equiv) in toluene (10 mL) were added tris(dibenzylideneacetone)dipalladium(0) (0.54 g, 0.59 mmol, 0.1 equiv) and BINAP (0.74 g, 1.2 mmol, 0.2 equiv). The resulting mixture was stirred overnight at 120° C. under nitrogen atmosphere. The mixture was then cooled to room temperature, concentrated under reduced pressure, and purified by silica gel column chromatography using a 0-5% gradient of methanol in dichloromethane to afford 1-[1-(3-bromo-2-fluorophenyl)piperidin-4-yl]pyrrolidin-2-one (860 mg, 31%) as a brown oil. MS (ESI) calculated for C₁₅H₁₈BrFN₂O: 340.06 m/z, found 341.00 [M+H]⁺.

### Step 2: Synthesis of 2-fluoro-3-[4-(2-oxopyrrolidin-1-yl)piperidin-1-yl]phenylboronic acid (Intermediate 42-1)

To a solution of 1-[1-(3-bromo-2-fluorophenyl)piperidin-4-yl]pyrrolidin-2-one (860 mg, 2.52 mmol, 1 equiv) and bis(pinacolato)diboron (448 mg, 1.76 mmol, 0.7 equiv) in 1,4-dioxane (10 mL) were added potassium acetate (742 mg, 7.56 mmol, 3 equiv) and [1,1′-bis(diphenylphosphino)ferrocene]dichloropalladium(II) (184 mg, 0.252 mmol, 0.1 equiv). The resulting mixture was stirred overnight at 120° C. under a nitrogen atmosphere, cooled to room temperature and concentrated under reduced pressure. The residue was purified by reverse-phase flash chromatography on C18 silica gel using a 10-50% gradient of acetonitrile in water (+0.05% ammonium bicarbonate) to provide 2-fluoro-3-[4-(2-oxopyrrolidin-1-yl)piperidin-1-yl]phenylboronic acid (Intermediate 42-1) (180 mg, 20%) as a white oil. MS (ESI) calculated for C₁₅H₂₀BFN₂O₃: 306.16 m/z, found 307.10 [M+H]⁺.

### Example 43: N-(4-(2-(2-aminopyridin-3-yl)-3H-imidazo[4,5-b]pyridin-3-yl)benzyl)-2-(3-(3,3-dimethylureido)-4-formylphenyl)acetamide

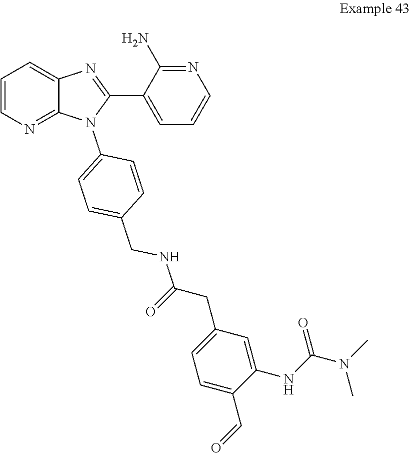 [SMILES: CN(C)C(=O)Nc1cc(CC(=O)NCc2ccc(-n3c(-c4cccnc4N)nc4cccnc43)cc2)ccc1C=O]

### Synthetic Route:

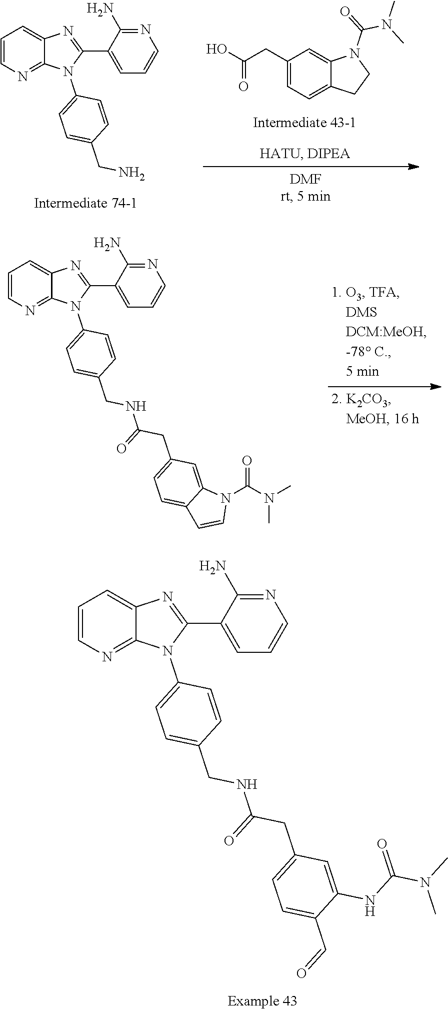 [SMILES: NCc1ccc(-n2c(-c3cccnc3N)nc3cccnc32)cc1 ; CN(C)C(=O)N1CCc2ccc(CC(=O)O)cc21 ; CN(C)C(=O)n1ccc2ccc(CC(=O)NCc3ccc(-n4c(-c5cccnc5N)nc5cccnc54)cc3)cc21 ; CN(C)C(=O)Nc1cc(CC(=O)NCc2ccc(-n3c(-c4cccnc4N)nc4cccnc43)cc2)ccc1C=O]

### Step 1: Synthesis of 6-(2-((4-(2-(2-aminopyridin-3-yl)-3H-imidazo[4,5-b]pyridin-3-yl) benzyl)amino)-2-oxoethyl)-N,N-dimethyl-1H-indole-1-carboxamide

To a suspension of 3-(3-(4-(aminomethyl)phenyl)-3H-imidazo[4,5-b]pyridin-2-yl)pyridin-2-amine (Intermediate 74-1) (120 mg, 231 μmol, 1 equiv), HATU (108 mg, 278 μmol, 1.2 equiv) and 2-(1-(dimethylcarbamoyl)-1H-indol-6-yl)acetic acid (Intermediate 43-1) (70.4 mg, 243 μmol, 1.05 equiv) in N,N-dimethylformamide (1.9 mL) was added N,N-diisopropylethylamine (162 μL, 925 μmol, 4 equiv) and the mixture was stirred at room temperature for 5 min. The reaction mixture was then diluted with dichloromethane (10 mL) and water (10 mL). The layers were separated, and the aqueous layer was extracted with dichloromethane (10 mL). The organic layers were combined, dried over sodium sulfate, filtered, and concentrated in vacuo. The crude product was purified by silica gel column chromatography using a 0-10% gradient of methanol in dichloromethane to give 6-(2-((4-(2-(2-aminopyridin-3-yl)-3H-imidazo[4,5-b]pyridin-3-yl)benzyl)amino)-2-oxoethyl)-N,N-dimethyl-1H-indole-1-carboxamide (78.5 mg, 62%) as a yellow oil. MS (ESI) calculated for C₃₁H₂₈N₈O₂: 544.23 m/z, found 545.42 [M+H]⁺.

### Step 2: Synthesis of N-(4-(2-(2-aminopyridin-3-yl)-3H-imidazo[4,5-b]pyridin-3-yl)benzyl)-2-(3-(3,3-dimethylureido)-4-formylphenyl)acetamide (Example 43)

To a solution of 6-(2-((4-(2-(2-aminopyridin-3-yl)-3H-imidazo[4,5-b]pyridin-3-yl)benzyl)amino)-2-oxoethyl)-N,N-dimethyl-1H-indole-1-carboxamide (60 mg, 110 μmol), in dichloromethane (5 mL) and methanol (500 μL) was added trifluoroacetic acid (17 μL, 220 μmol, 2 equiv) and the mixture was cooled to −78° C. Ozone was then bubbled through the solution for 5 min. The reaction mixture was sparged with nitrogen for 5 min, then quenched with dimethyl sulfide (16.3 μL, 220 μmol, 2 equiv). The mixture was allowed to warm to 0° C. and stirring was continued for 1 h. The reaction mixture was then concentrated in vacuo, coevaporating with dichloromethane (3×10 mL). Potassium carbonate (60.9 mg, 441 μmol, 4 equiv) and methanol (5 mL) were added to the residue and the mixture was stirred at room temperature overnight. The reaction mixture was quenched with 1M hydrochloric acid (5 mL), and the mixture was concentrated in vacuo. The residue was purified by Preparative HPLC on a CSH C18 column using a 10-30% gradient of acetonitrile in water (+0.1% formic acid) to give N-(4-(2-(2-aminopyridin-3-yl)-3H-imidazo[4,5-b]pyridin-3-yl)benzyl)-2-(3-(3,3-dimethylureido)-4-formylphenyl) acetamide (Example 43) (20.2 mg, 33%) as a white solid. MS (ESI) calculated for C₃₀H₂₈N₈O₃: 548.23 m/z, found 549.41 [M+H]⁺. ¹H NMR (400 MHz, DMSO-d₆) δ (ppm): 10.64 (s, 1H), 9.92 (s, 1H), 8.75 (t, J=5.9 Hz, 1H), 8.43 (s, 1H), 8.31 (dd, J=4.7, 1.3 Hz, 1H), 8.20 (dd, J=8.0, 1.3 Hz, 1H), 7.98 (dd, J=4.8, 1.8 Hz, 1H), 7.78 (d, J=7.9 Hz, 1H), 7.44-7.33 (m, 5H), 7.21 (dd, J=7.7, 1.7 Hz, 1H), 7.10 (dd, J=7.9, 1.2 Hz, 1H), 6.97 (s, 2H), 6.39 (dd, J=7.7, 4.8 Hz, 1H), 4.37 (d, J=5.9 Hz, 2H), 3.58 (s, 2H), 2.98 (s, 6H).

### Intermediate 43-1: 2-(1-(dimethylcarbamoyl)-1H-indol-6-yl)acetic acid

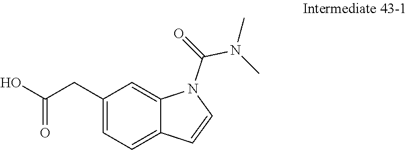 [SMILES: CN(C)C(=O)n1ccc2ccc(CC(=O)O)cc21]

### Synthetic Route:

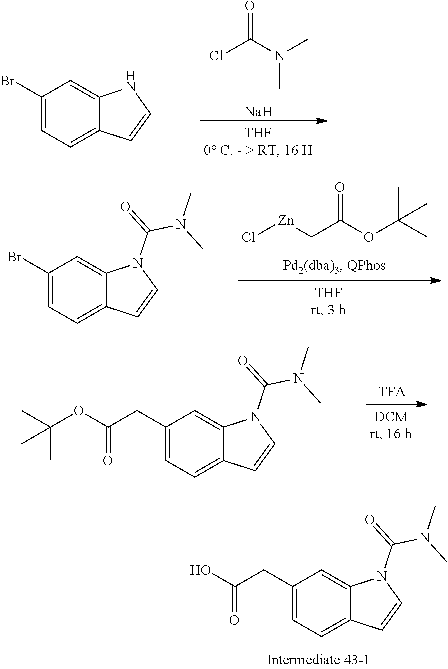

### Step 1: Synthesis of 6-bromo-N,N-dimethyl-1H-indole-1-carboxamide

To a cooled (0° C.) suspension of sodium hydride (240 mg, 6.00 mmol, 1.2 equiv) in tetrahydrofuran (6 mL) was added a solution of 6-bromoindole (1.00 g, 5.00 mmol, 1 equiv) in tetrahydrofuran (4 mL) and the resulting dark brown solution was stirred at 0° C. for 5 min. The mixture was then stirred at room temperature for 1 h then cooled to 0° C. Dimethylcarbamoyl chloride (517 μL, 5.50 mmol, 1.1 equiv) was added dropwise and the resulting mixture was stirred overnight at room temperature. The reaction was then quenched with saturated aqueous ammonium chloride (10 mL) and water (10 mL) and diluted with ethyl acetate (20 mL). The layers were separated, and the aqueous layer was extracted with ethyl acetate (10 mL). The organic layers were combined, washed with brine (20 mL), then dried over sodium sulfate, filtered, and concentrated in vacuo. The crude product was purified by silica gel column chromatography using a 0-25% gradient of ethyl acetate in hexanes to give 6-bromo-N,N-dimethyl-1H-indole-1-carboxamide (1.12 g, 84%) as a red oil. MS (ESI) calculated for C₁₁H₁₁BrN₂O: 266.01 m/z, found 267.06, 269.04 [M+H, M+2+H]⁺.

### Step 2: Synthesis of tert-butyl 2-(1-(dimethylcarbamoyl)-1H-indol-6-yl)acetate

To a solution of 6-bromo-N,N-dimethyl-1H-indole-1-carboxamide (1.16 g, 4.34 mmol, 1 equiv) in tetrahydrofuran (8.69 mL) was added QPhos (157 mg, 217 μmol, 0.05 equiv) and tris(dibenzylideneacetone)dipalladium(0) (203 mg, 217 μmol, 0.05 equiv) and the resulting solution was sparged with nitrogen for 5 mins. 2-(tert-butoxy)-2-oxoethylzinc chloride (0.5 M in diethyl ether, 13.0 mL, 6.51 mmol, 1.5 equiv) was then added to the solution and the mixture was stirred at room temperature for 3 h under a nitrogen atmosphere. The reaction mixture was then concentrated in vacuo to give tert-butyl 2-(1-(dimethylcarbamoyl)-1H-indol-6-yl)acetate as a red oil, which was used directly in the next step without purification. MS (ESI) calculated for C₇H₂₂N₂O₃: 302.16 m/z, found 301.11 [M−H]⁻.

### Step 3: Synthesis of 2-(1-(dimethylcarbamoyl)-1H-indol-6-yl)acetic acid (Intermediate 43-1)

To a solution of crude tert-butyl 2-(1-(dimethylcarbamoyl)-1H-indol-6-yl)acetate (1.31 g, 4.34 mmol) in dichloromethane (17 mL) was added trifluoroacetic acid (10.1 mL) and the mixture was stirred for 10 min at room temperature. The reaction mixture was then concentrated in vacuo, co-evaporating with dichloromethane (2×20 mL). The residue was then redissolved in dichloromethane (20 mL) and extracted with 2M aqueous sodium hydroxide (50 mL). The aqueous layer was then acidified with 6M aqueous hydrochloric acid (15 mL) and extracted with ethyl acetate (2×30 mL). The organic layers were combined, dried over sodium sulfate, filtered, and concentrated in vacuo. The crude product was purified by silica gel column chromatography using a 0-5% gradient of methanol in dichloromethane to give 2-(1-(dimethylcarbamoyl)-1H-indol-6-yl)acetic acid (Intermediate 43-1) (327 mg, 31% over two steps) as a yellow oil. MS (ESI) calculated for C₁₃H₁₄N₂O₃: 246.10 m/z, found 247.15 [M+H]⁺.

### Example 44: N-(5-(2-((4-(2-(2-aminopyridin-3-yl)-3H-imidazo[4,5-b]pyridin-3-yl)benzyl)amino)-2-oxoethyl)-2-formylphenyl)-1-methyl-1H-pyrazole-4-carboxamide

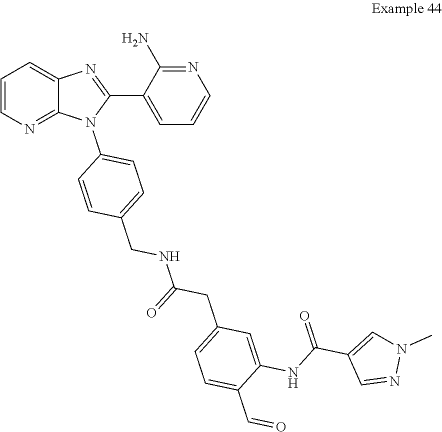 [SMILES: Cn1cc(C(=O)Nc2cc(CC(=O)NCc3ccc(-n4c(-c5cccnc5N)nc5cccnc54)cc3)ccc2C=O)cn1]

Example 44 was prepared in a manner analogous to Example 43 using Intermediate 44-2 in place of Intermediate 43-1. MS (ESI) calculated for C₃₂H₂₇N₉03: 585.22 m/z, found 585.83 [M+H]⁺. ¹H NMR (400 MHz, DMSO-d₆) δ (ppm): 11.33 (s, 1H), 9.97 (s, 1H), 8.78 (t, J=5.9 Hz, 1H), 8.36 (s, 1H), 8.32 (s, 1H), 8.30 (dd, J=4.8, 1.4 Hz, 1H), 8.20 (dd, J=8.0, 1.4 Hz, 1H), 7.98 (dd, J=4.8, 1.8 Hz, 1H), 7.93 (s, 1H), 7.86 (d, J=7.9 Hz, 1H), 7.43-7.33 (m, 5H), 7.26 (dd, J=7.9, 1.3 Hz, 1H), 7.22 (dd, J=7.6, 1.8 Hz, 1H), 6.98 (s, 2H), 6.39 (dd, J=7.7, 4.9 Hz, 1H), 4.38 (d, J=5.9 Hz, 2H), 3.92 (s, 3H), 3.64 (s, 2H).

### Intermediate 44-2: 2-(1-(1-methyl-1H-pyrazole-4-carbonyl)-1H-indol-6-yl)acetic acid

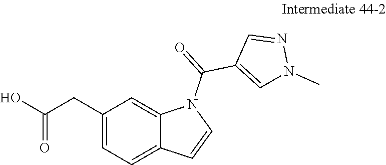 [SMILES: Cn1cc(C(=O)n2ccc3ccc(CC(=O)O)cc32)cn1]

Intermediate 44-2 was prepared in a manner analogous to Intermediate 43-1 (starting from Step 2) using Intermediate 44-1 in place of the bromide starting material. MS (ESI) calculated for C₁₅H₁₃N₃O₃: 283.10 m/z, found 283.86 [M+H]⁺.

### Intermediate 44-1: (6-iodo-1H-indol-1-yl)(1-methyl-1H-pyrazol-4-yl)methanone

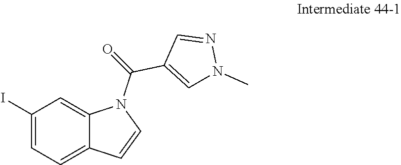 [SMILES: Cn1cc(C(=O)n2ccc3ccc(I)cc32)cn1]

### Synthetic Route:

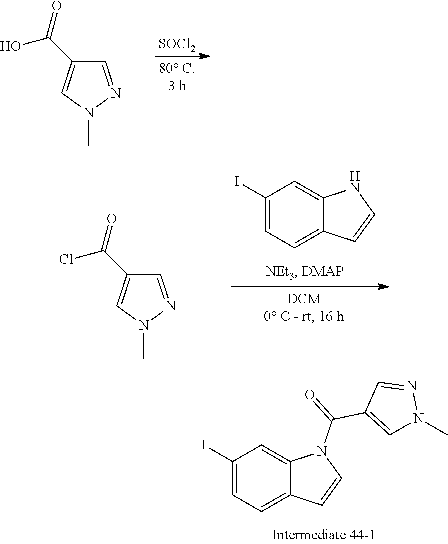

### Step 1: Synthesis of 1-methyl-1H-pyrazole-4-carbonyl chloride

1-Methyl-1H-pyrazole-4-carboxylic acid (750 mg, 5.83 mmol) was dissolved in thionyl chloride (2.56 mL, 35.0 mmol, 6 equiv) and the mixture was heated to 80° C. for 3 h. The reaction mixture was then concentrated in vacuo to give 1-methyl-1H-pyrazole-4-carbonyl chloride (842 mg, crude quant) as a brown solid, which was used directly in the next step without further purification.

### Step 2: Synthesis of (6-iodo-1H-indol-1-yl)(1-methyl-1H-pyrazol-4-yl)methanone (Intermediate 44-1)

To a solution of 6-iodo-1H-indole (1.13 g, 4.65 mmol, 1 equiv) and 4-dimethylaminopyridine (58.0 mg, 465 μmol, 0.1 equiv) in dichloromethane (18.6 mL) was added triethylamine (977 μL, 6.97 mmol, 1.5 equiv) and the solution was cooled to 0° C. 1-methyl-1H-pyrazole-4-carbonyl chloride (840 mg, 5.81 mmol, 1.25 equiv) was added to the mixture and the reaction was slowly allowed to reach room temperature and stirred overnight. The reaction mixture was then washed with saturated aqueous ammonium chloride (2×15 mL) and brine (15 mL), then dried over sodium sulfate, filtered, and concentrated in vacuo. The crude product was purified by silica gel column chromatography using a 30-100% gradient of ethyl acetate in hexanes to give (6-iodo-1H-indol-1-yl)(1-methyl-1H-pyrazol-4-yl)-methanone (Intermediate 44-1) (1.39 g, 85% over two steps) as a brown solid. MS (ESI) calculated for C₁₃H₁₀IN₃O: 350.99 m/z, found 352.15 [M+H]⁺.

### Example 45: N-(4-(2-(2-aminopyridin-3-yl)-3H-imidazo[4,5-b]pyridin-3-yl)benzyl)-4-formyl-5-hydroxypicolinamide

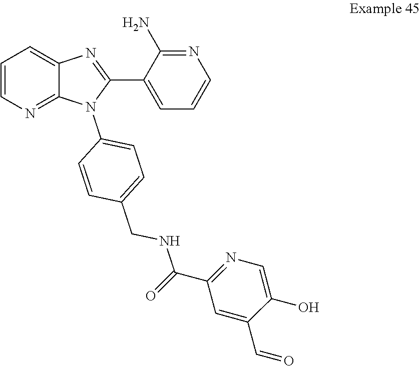 [SMILES: Nc1ncccc1-c1nc2cccnc2n1-c1ccc(CNC(=O)c2cc(C=O)c(O)cn2)cc1]

Example 45 was prepared in a manner analogous to Example 43 using furo[2,3-c]pyridine-5-carboxylic acid in place of Intermediate 43-1. MS (ESI) calculated for C₂₅H₁₉N₇O₃: 465.15 m/z, found 465.80 [M+H]⁺. ¹H NMR (400 MHz, methanol-d₄) δ (ppm): 10.15 (s, 1H), 8.61 (s, 1H), 8.50 (dd, J=4.9, 1.3 Hz, 1H), 8.46 (s, 1H), 8.33 (dt, J=17.2, 8.6 Hz, 1H), 8.12 (dt, J=13.2, 6.6 Hz, 1H), 7.77 (dt, J=10.7, 5.3 Hz, 1H), 7.64 (d, J=8.3 Hz, 2H), 7.55-7.45 (m, 3H), 6.75 (dt, J=24.4, 12.2 Hz, 1H), 4.83 (s, 2H).

### Example 46: N-(4-(2-(2-aminopyridin-3-yl)-3H-imidazo[4,5-b]pyridin-3-yl)benzyl)-5-formyl-6-hydroxynicotinamide

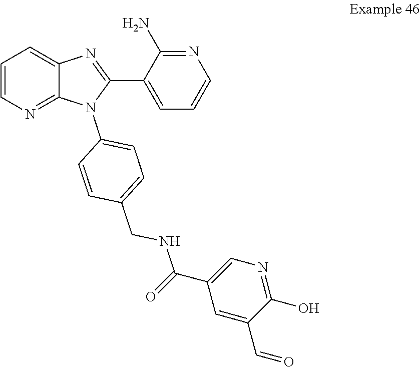 [SMILES: Nc1ncccc1-c1nc2cccnc2n1-c1ccc(CNC(=O)c2cnc(O)c(C=O)c2)cc1]

Example 46 was prepared in a manner analogous to Example 43 using furo[2,3-b]pyridine-5-carboxylic acid in place of Intermediate 43-1 and omitting the potassium carbonate/methanol step. MS (ESI) calculated for C₂₅H₁₉N₇O₃: 465.15 m/z, found 465.85 [M+H]⁺. ¹H NMR (400 MHz, methanol-d₄) δ (ppm): 10.18 (s, 1H), 8.74-8.68 (m, 1H), 8.67-8.59 (m, 1H), 8.47 (t, J=10.3 Hz, 1H), 8.31 (d, J=11.5 Hz, 1H), 8.10 (d, J=5.6 Hz, 1H), 7.76 (d, J=7.4 Hz, 1H), 7.68-7.58 (m, 2H), 7.54-7.44 (m, 3H), 6.81-6.70 (m, 1H), 4.77 (s, 2H).

### Example 47: N-(4-(2-(2-aminopyridin-3-yl)-3H-imidazo[4,5-b]pyridin-3-yl)benzyl)-6-formyl-5-hydroxypicolinamide

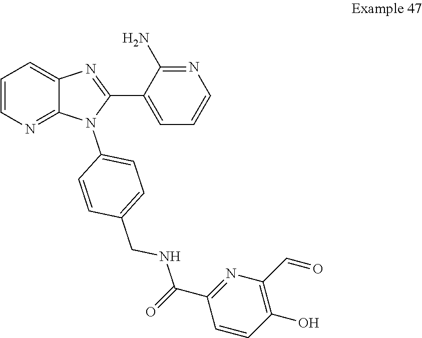 [SMILES: Nc1ncccc1-c1nc2cccnc2n1-c1ccc(CNC(=O)c2ccc(O)c(C=O)n2)cc1]

Example 47 was prepared in a manner analogous to Example 43 using furo[3,2-b]pyridine-5-carboxylic acid in place of Intermediate 43-1 and omitting the potassium carbonate/methanol step. MS (ESI) calculated for C₂₅H₁₉N₇O₃: 465.15 m/z, found 465.85 [M+H]⁺. ¹H NMR (400 MHz, methanol-d₄) δ (ppm): 10.01 (d, J=18.4 Hz, 1H), 8.47 (dt, J=8.1, 4.0 Hz, 1H), 8.39 (d, J=7.4 Hz, 1H), 8.31 (dt, J=14.9, 7.5 Hz, 1H), 8.09 (dt, J=13.6, 6.7 Hz, 1H), 7.75 (dt, J=6.0, 3.0 Hz, 1H), 7.62 (d, J=8.4 Hz, 2H), 7.57 (dd, J=11.0, 5.9 Hz, 1H), 7.53-7.43 (m, 3H), 6.77-6.69 (m, 1H), 4.81 (d, J=9.9 Hz, 2H).

### Example 48: N-(5-(2-((4-(2-(2-aminopyridin-3-yl)-3H-imidazo[4,5-b]pyridin-3-yl)benzyl)amino)-2-oxoethyl)-2-formylphenyl)cyclopropanecarboxamide

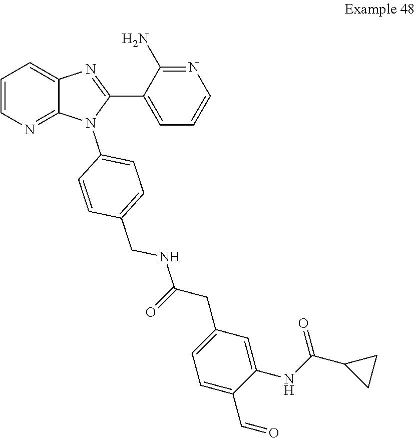 [SMILES: Nc1ncccc1-c1nc2cccnc2n1-c1ccc(CNC(=O)Cc2ccc(C=O)c(NC(=O)C3CC3)c2)cc1]

Example 48 was prepared in a manner analogous to Example 43 using Intermediate 48-2 in place of Intermediate 43-1 and EDCI (2 equiv)/pyridine (20 equiv)/48 h in place of HATU/N,N-diisopropylethylamine/5 min. MS (ESI) calculated for C₃₁H₂₇N₇O₃: 545.22 m/z, found 546.39 [M+H]⁺. ¹H NMR (400 MHz, methanol-d₄) δ (ppm): 9.94 (s, 1H), 8.54 (s, 1H), 8.31 (dd, J=10.7, 9.7 Hz, 1H), 8.18 (dt, J=17.0, 8.4 Hz, 1H), 8.00-7.90 (m, 1H), 7.79 (d, J=7.9 Hz, 1H), 7.52-7.39 (m, 3H), 7.36 (d, J=8.3 Hz, 2H), 7.32-7.19 (m, 2H), 6.41 (dt, J=24.6, 12.3 Hz, 1H), 4.48 (s, 2H), 3.66 (s, 2H), 1.74 (ddd, J=12.5, 7.7, 4.6 Hz, 1H), 1.02-0.85 (m, 4H).

### Intermediate 48-2: 2-(1-(cyclopropanecarbonyl)-1H-indol-6-yl)acetic acid

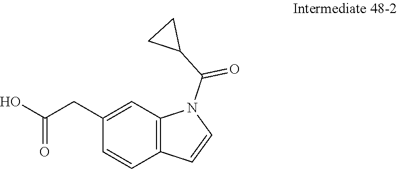 [SMILES: O=C(O)Cc1ccc2ccn(C(=O)C3CC3)c2c1]

Intermediate 48-2 was prepared in a manner analogous to Intermediate 43-1 (starting from Step 2) using Intermediate 48-1 in place of the bromide starting material. MS (ESI) calculated for C₄H₁₃NO₃: 243.09 m/z, found 244.14 [M+H]⁺.

### Intermediate 48-1: (6-bromo-1H-indol-1-yl)(cyclopropyl)methanone

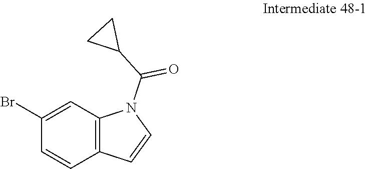 [SMILES: O=C(C1CC1)n1ccc2ccc(Br)cc21]

### Synthetic Route:

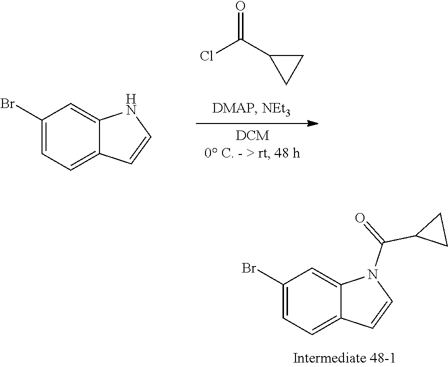 [SMILES: O=C(Cl)C1CC1 ; Brc1ccc2cc[nH]c2c1 ; O=C(C1CC1)n1ccc2ccc(Br)cc21]

Step 1: Synthesis of (6-bromo-1H-indol-1-yl)(cyclopropyl)methanone (Intermediate 48-1)

To a solution of 6-bromoindole (500 mg, 2.50 mmol, 1 equiv) and 4-dimethylaminopyridine (31.2 mg, 250 μmol, 1 equiv) in dichloromethane (10.0 mL) was added triethylamine (525 μL, 3.75 mmol, 1.5 equiv) and the solution was cooled to 0° C. Cyclopropanecarbonyl chloride (289 μL, 3.12 mmol, 1.25 equiv) was then added and the reaction was allowed to warm to room temperature and stirred for 48 h. The reaction was then quenched with saturated aqueous ammonium chloride (10 mL) and water (10 mL) and diluted with dichloromethane (20 mL). The layers were separated, and the organic layer was washed again with saturated aqueous ammonium chloride (20 mL). The organic layer was then dried over sodium sulfate, filtered, and concentrated in vacuo to give (6-bromo-1H-indol-1-yl)(cyclopropyl)-methanone (Intermediate 48-1) (660 mg, crude) as a brown solid, which was used in subsequent transformations without further purification. MS (ESI) calculated for C₁₂H₁₀BrNO: 262.99 m/z, found 261.88, 262.88 [M−H]⁻.

### Example 49: N-({4-[2-(2-aminopyridin-3-yl)-5-(morpholin-4-yl)imidazo[4,5-b]pyridin-3-yl]phenyl}methyl)-2-(4-formyl-3-hydroxyphenyl)acetamide

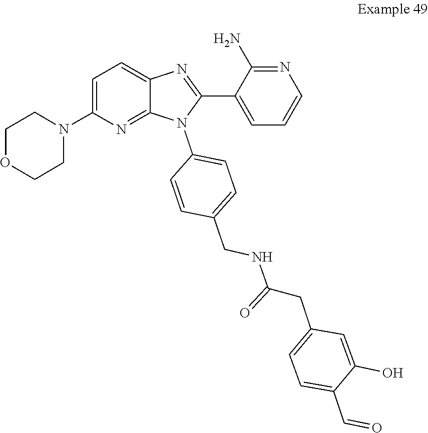 [SMILES: Nc1ncccc1-c1nc2ccc(N3CCOCC3)nc2n1-c1ccc(CNC(=O)Cc2ccc(C=O)c(O)c2)cc1]

Example 49 was prepared in a manner analogous to Example 1 (starting from Step 3) using Intermediate 3-1 in place of the amine starting material. MS (ESI) calculated for C₃₁H₂₉N₇O₄: 563.23 m/z, found 564.25 [M+H]⁺. ¹H NMR (300 MHz, DMSO-d₆) δ (ppm): 10.18 (s, 1H), 7.93-8.00 (m, 2H), 7.57-7.60 (m, 1H), 7.28-7.36 (m, 4H), 7.03-7.06 (m, 1H), 6.84-6.91 (m, 3H), 6.34-6.38 (m, 1H), 4.37 (s, 2H), 3.64-3.72 (m, 6H), 3.39-3.41 (m, 4H).

### Example 50: N-({4-[2-(2-aminopyridin-3-yl)-5-phenylimidazo[4,5-b]pyridin-3-yl]phenyl}methyl)-3-bromo-5-formyl-4-hydroxybenzamide

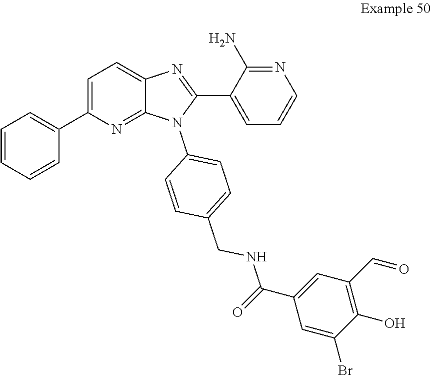 [SMILES: Nc1ncccc1-c1nc2ccc(-c3ccccc3)nc2n1-c1ccc(CNC(=O)c2cc(Br)c(O)c(C=O)c2)cc1]

### Synthetic Route:

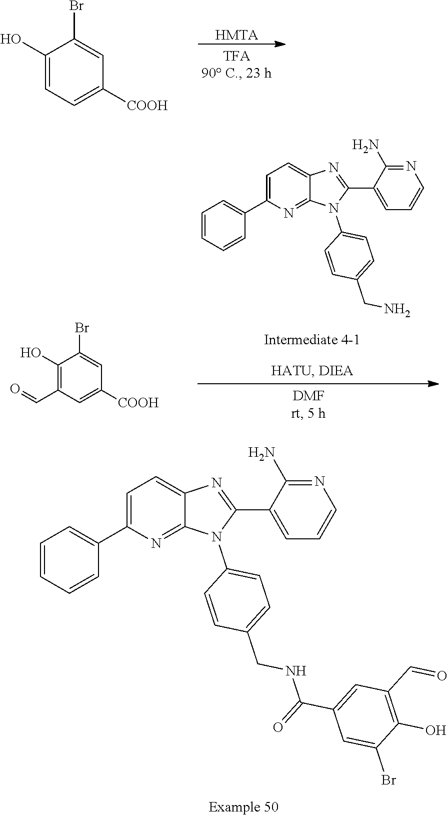

### Step 1: Synthesis of 3-bromo-5-formyl-4-hydroxybenzoic acid

To a solution of 3-bromo-4-hydroxybenzoic acid (2.00 g, 9.22 mmol, 1 equiv) in 2,2,2-trifluoroacetic acid (5 mL) was added a solution of 1,3,5,7-tetraazaadamantane (3.59 g, 25.6 mmol, 2 equiv) in 2,2,2-trifluoroacetic acid (5 mL) dropwise and the obtained solution was stirred at 90° C. for 23 h. After cooling to room temperature, 150 mL of water was added, and the resulted mixture was acidified with 4N aqueous hydrochloric acid giving a precipitate. The precipitate was collected by filtration, washed with water, and dried in an oven to provide 3-bromo-5-formyl-4-hydroxybenzoic acid (2.5 g, 96%) as a brown solid, which was used directly in the next step without further purification. MS (ESI) calculated for C₈H₅BrO₄: 243.94 m/z, found 246.90 [M+H+2]⁺.

### Step 2: Synthesis of N-({4-[2-(2-aminopyridin-3-yl)-5-phenylimidazo[4,5-b]pyridin-3-yl]phenyl}methyl)-3-bromo-5-formyl-4-hydroxybenzamide (Example 50)

A solution of 3-bromo-5-formyl-4-hydroxybenzoic acid (187 mg, 0.765 mmol, 1.5 equiv), 3-{3-[4-(aminomethyl)phenyl]-5-phenylimidazo[4,5-b]pyridin-2-yl}pyridin-2-amine (Intermediate 4-1) (200 mg, 0.510 mmol, 1.00 equiv), N,N-diisopropylethylamine (263 mg, 2.04 mmol, 4 equiv) and HATU (291 mg, 0.765 mmol, 1.5 equiv) in N,N-dimethylformamide (5 mL) was stirred at room temperature for 2 h. The solution was purified by preparative HPLC on a XBridge Prep OBD C18 Column using a 17-42% gradient of acetonitrile in water (+10 mmol/L ammonium bicarbonate) to provide N-({4-[2-(2-aminopyridin-3-yl)-5-phenylimidazo[4,5-b]pyridin-3-yl]phenyl}methyl)-3-bromo-5-formyl-4-hydroxybenzamide (Example 50) (8.3 mg, 2%) as a light-yellow solid. MS (ESI) calculated for C₃₂H₂₃BrN₆O₃: 618.10 m/z, found 618.95, 620.95 [M+H, M+2+H]⁺. ¹H NMR (300 MHz, DMSO-d₆) δ (ppm): 10.13 (s, 1H), 9.43 (s, 1H), 8.23-8.56 (m, 1H), 7.92-8.82 (m, 6H), 7.36-7.55 (m, 8H), 7.21-7.24 (m, 1H), 4.53 (s, 2H).

### Example 51: N-(4-(2-(2-aminopyridin-3-yl)-5-(3-hydroxy-3-methylbut-1-yn-1-yl)-3H-imidazo[4,5-b]pyridin-3-yl)benzyl)-2-(4-formyl-3-hydroxyphenyl)acetamide

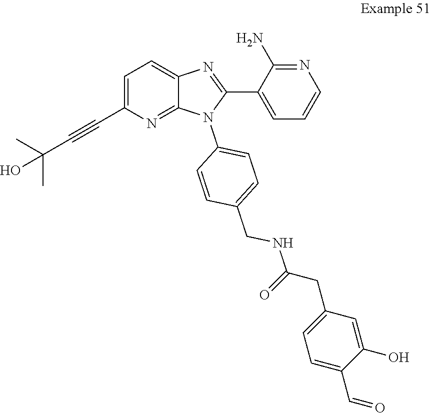 [SMILES: CC(C)(O)C#Cc1ccc2nc(-c3cccnc3N)n(-c3ccc(CNC(=O)Cc4ccc(C=O)c(O)c4)cc3)c2n1]

Example 51 was prepared in a manner analogous to Example 1 (starting from Step 3) using Intermediate 51-2 in place of the amine starting material. MS (ESI) calculated for C₃₂H₂₈N₆O₄: 560.22 m/z, found 561.25 [M+H]⁺. ¹H NMR (300 MHz, DMSO-d₆) δ (ppm): 10.18 (s, 1H), 8.20-8.23 (m, 1H), 7.62-8.04 (m, 1H), 7.41-7.60 (m, 7H), 6.90-6.95 (m, 2H), 6.65-6.69 (m, 1H), 4.37 (s, 2H), 3.55 (s, 2H), 1.54 (s, 6H).

### Intermediate 51-2: 4-(3-(4-(aminomethyl)phenyl)-2-(2-aminopyridin-3-yl)-3H-imidazo[4,5-b]pyridin-5-yl)-2-methylbut-3-yn-2-ol

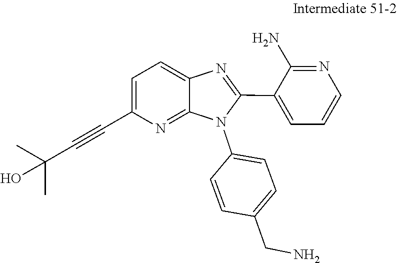 [SMILES: CC(C)(O)C#Cc1ccc2nc(-c3cccnc3N)n(-c3ccc(CN)cc3)c2n1]

Intermediate 51-2 was prepared in a manner analogous to Intermediate 11-1 using Intermediate 51-1 in place of 2-chloro-3-nitro-6-phenylpyridine and tert-butyl N-[(4-aminophenyl)methyl]carbamate in place of tert-butyl 2-(4-aminophenyl)acetate. MS (ESI) calculated for C₂₃H₂₂N₆O: 398.19 m/z, found 399.13 [M+H]⁺.

### Intermediate 51-1: 4-(6-chloro-5-nitropyridin-2-yl)-2-methylbut-3-yn-2-ol

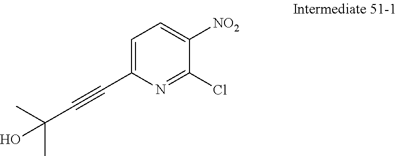 [SMILES: CC(C)(O)C#Cc1ccc([N+](=O)[O-])c(Cl)n1]

### Synthetic Route:

### Step 1: Synthesis of 4-(6-chloro-5-nitropyridin-2-yl)-2-methylbut-3-yn-2-ol (Intermediate 51-1)

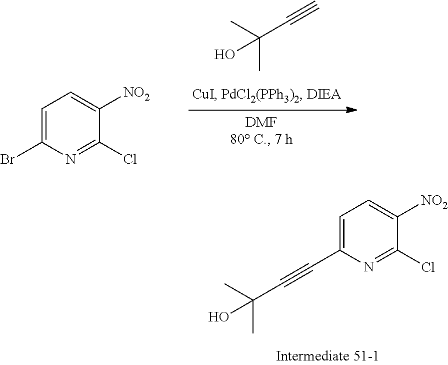

To a stirred solution of 6-bromo-2-chloro-3-nitropyridine (1.00 g, 4.21 mmol, 1 equiv) in anhydrous N,N-dimethylformamide (14 mL) were added 2-methyl-3-butyn-2-ol (0.53 g, 6.3 mmol, 1.5 equiv), copper(I) iodide (0.64 g, 3.4 mmol, 0.8 equiv), bis(triphenylphosphine)palladium(II) dichloride (0.15 g, 0.21 mmol, 0.05 equiv) and N,N-diisopropylethylamine (1.36 g, 10.5 mmol, 2.5 equiv). The mixture was stirred at 80° C. for 7 h under nitrogen atmosphere. Water was added and the resulting mixture was extracted with ethyl acetate (3×60 mL). The combined organic layers were washed with brine (2×40 mL), dried over anhydrous sodium sulfate, filtered, and concentrated in vacuo. The residue was purified by silica gel column chromatography using a 0-95% gradient of ethyl acetate in petroleum ether to afford 4-(6-chloro-5-nitropyridin-2-yl)-2-methylbut-3-yn-2-ol (Intermediate 51-1) (700 mg, 62%) as a brown solid. MS (ESI) calculated for C₁₀H₉ClN₂O₃: 240.03 m/z, found 241.06 [M+H]⁺.

### Example 52: N-(4-(2-(2-aminopyridin-3-yl)-5-phenyl-3H-imidazo[4,5-b]pyridin-3-yl)benzyl)-3-chloro-5-formyl-4-hydroxybenzamide

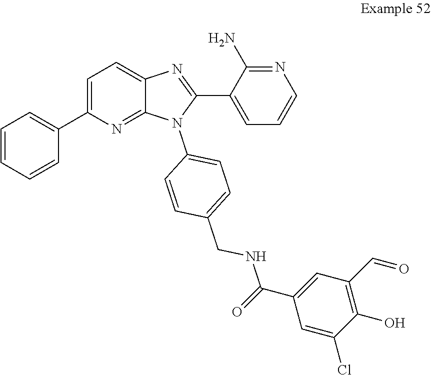 [SMILES: Nc1ncccc1-c1nc2ccc(-c3ccccc3)nc2n1-c1ccc(CNC(=O)c2cc(Cl)c(O)c(C=O)c2)cc1]

Example 52 was prepared in a manner analogous to Example 50 using 3-chloro-4-hydroxybenzoic acid in place of 3-bromo-4-hydroxybenzoic acid. MS (ESI) calculated for C₃₂H₂₃ClN₆O₃: 574.15 m/z, found 575.10 [M+H]⁺. ¹H NMR (400 MHz, DMSO-d₆) δ (ppm) 10.19 (s, 1H), 9.08 (s, 1H), 8.12-8.33 (m, 2H), 7.88-8.12 (m, 5H), 7.34-7.58 (m, 8H), 7.18-7.30 (m, 1H), 6.95 (s, 2H), 6.38-6.52 (m, 1H), 4.45-4.68 (m, 2H).

### Example 53: N-({4-[2-(2-aminopyridin-3-yl)-5-phenylimidazo[4,5-b]pyridin-3-yl]phenyl}methyl)-3-fluoro-5-formyl-4-hydroxybenzamide

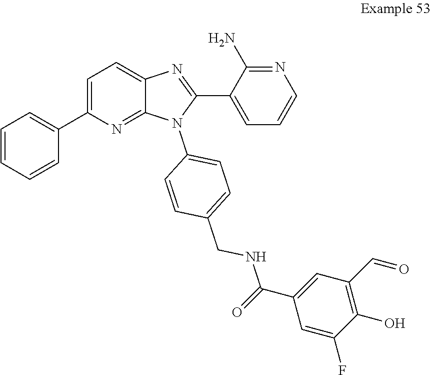 [SMILES: Nc1ncccc1-c1nc2ccc(-c3ccccc3)nc2n1-c1ccc(CNC(=O)c2cc(F)c(O)c(C=O)c2)cc1]

Example 53 was prepared in a manner analogous to Example 50 using 3-fluoro-4-hydroxybenzoic acid in place of 3-bromo-4-hydroxybenzoic acid. MS (ESI) calculated for C₃₂H₂₃FN₆O₃: 558.18 m/z, found 559.10 [M+H]⁺. ¹H NMR (400 MHz, DMSO-d₆) δ 10.29 (s, 1H), 9.10-9.21 (m, 1H), 8.26-8.28 (m, 1H), 8.03-8.09 (m, 1H), 7.91-8.01 (m, 5H), 7.40-7.49 (m, 7H), 7.22-7.24 (m, 1H), 6.42-6.45 (m, 1H), 4.57-4.59 (m, 2H). ¹⁹F NMR (400 MHz, DMSO-d₆) δ-134.54.

### Example 54: N-({4-[2-(2-aminopyridin-3-yl)-5-(2-fluorophenyl)imidazo[4,5-b]pyridin-3-yl]phenyl}methyl)-2-(4-formyl-3-hydroxyphenyl)acetamide

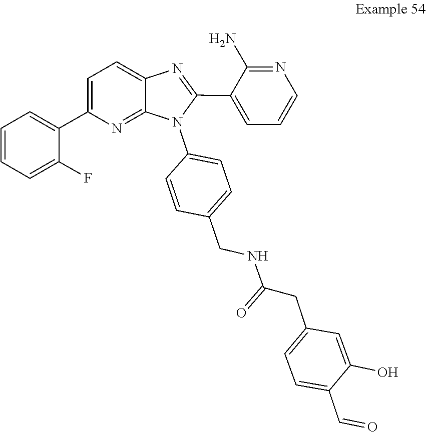 [SMILES: Nc1ncccc1-c1nc2ccc(-c3ccccc3F)nc2n1-c1ccc(CNC(=O)Cc2ccc(C=O)c(O)c2)cc1]

Example 54 was prepared in a manner analogous to Example 1 using 2-fluorophenylboronic acid in place of 4-(morpholin-4-yl)phenylboronic acid. MS (ESI) calculated for C₃₃H₂₅FN₆O₃: 572.20 m/z, found 573.20 [M+H]⁺. ¹H NMR (300 MHz, DMSO-d₆) δ (ppm): 10.18 (s, 1H), 8.28-8.31 (m, 1H), 8.00-8.02 (m, 1H), 7.76-7.78 (m, 2H), 7.59-7.62 (m, 1H), 7.21-7.45 (m, 9H), 6.88-6.95 (m, 2H), 6.41-6.45 (m, 1H), 4.37 (s, 2H), 3.52-3.55 (m, 2H).

### Example 55: N-({4-[2-(2-aminopyridin-3-yl)-5-[3-(morpholin-4-yl)phenyl]imidazo[4,5-b]pyridin-3-yl]phenyl}methyl)-2-(4-formyl-3-hydroxyphenyl)acetamide

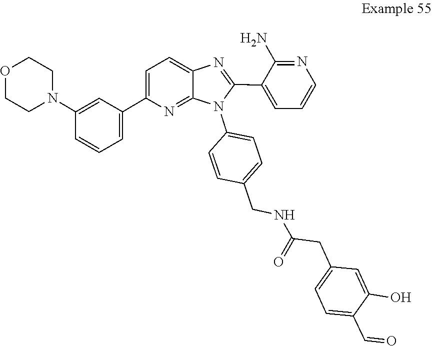 [SMILES: Nc1ncccc1-c1nc2ccc(-c3cccc(N4CCOCC4)c3)nc2n1-c1ccc(CNC(=O)Cc2ccc(C=O)c(O)c2)cc1]

Example 55 was prepared in a manner analogous to Example 1 (starting from Step 3) using Intermediate 12-1 in place of the amine starting material. MS (ESI) calculated for C₃₇H₃₃N₇O₄: 639.26 m/z, found 640.25 [M+H]⁺. ¹H NMR (300 MHz, DMSO-d₆) δ (ppm): 10.18 (s, 1H), 8.23-8.26 (m, 1H), 7.97-8.02 (m, 2H), 7.59-7.62 (m, 2H), 7.39-7.48 (m, 5H), 7.29-7.35 (m, 1H), 7.21-7.25 (m, 1H), 6.88-7.01 (m, 3H), 6.41-6.45 (m, 1H), 4.39 (s, 2H), 3.76-3.79 (m, 4H), 3.52 (s, 2H), 3.15-3.18 (m, 4H).

### Example 56: N-({4-[2-(2-aminopyridin-3-yl)-5-phenylimidazo[4,5-b]pyridin-3-yl]phenyl}methyl)-1-(6-formyl-5-hydroxypyridin-3-yl)cyclopropane-1-carboxamide

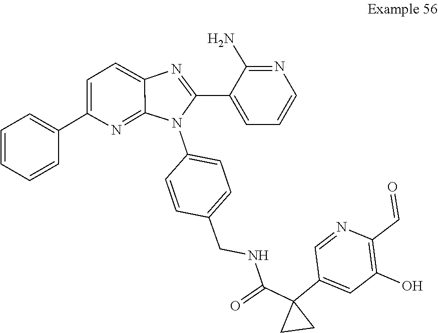 [SMILES: Nc1ncccc1-c1nc2ccc(-c3ccccc3)nc2n1-c1ccc(CNC(=O)C2(c3cnc(C=O)c(O)c3)CC2)cc1]

Example 56 was prepared in a manner analogous to Example 1 (starting from Step 3) using Intermediate 4-1 in place of the amine starting material and Intermediate 56-1 in place of Intermediate 1-4. MS (ESI) calculated for C₃₄H₂₇N₇O₃: 581.22 m/z, found 582.20 [M+H]⁺. ¹H NMR (400 MHz, DMSO-d₆) δ (ppm): 10.11 (s, 1H), 8.31-8.32 (m, 1H), 8.25-8.27 (m, 1H), 7.97-8.02 (m, 5H), 7.34-7.49 (m, 8H), 7.21-7.32 (m, 1H), 6.41-6.44 (m, 1H), 4.32-4.34 (m, 2H), 1.46-1.47 (m, 2H), 1.14-1.15 (m, 2H).

### Intermediate 56-1: 1-(6-(1,3-dioxolan-2-yl)-5-((4-methoxybenzyl)oxy)pyridin-3-yl)cyclopropane-1-carboxylic acid

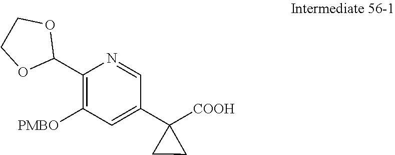

### Synthetic Route:

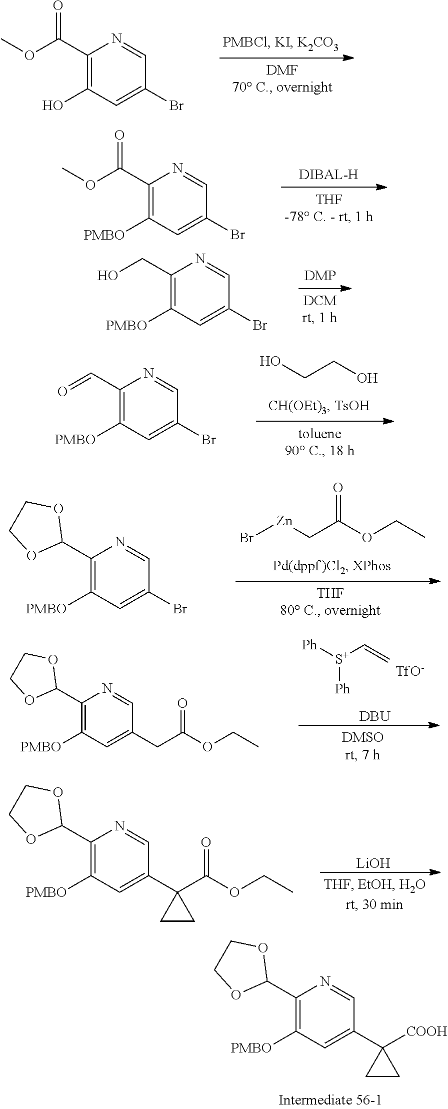

### Step 1: Synthesis of methyl 5-bromo-3-[(4-methoxyphenyl)methoxy]pyridine-2-carboxylate

A mixture of methyl 5-bromo-3-hydroxypyridine-2-carboxylate (5.00 g, 21.5 mmol, 1 equiv), p-methoxybenzyl chloride (4.05 g, 25.9 mmol, 1.2 equiv), potassium iodide (0.36 g, 2.2 mmol, 0.1 equiv) and potassium carbonate (8.93 g, 64.6 mmol, 3 equiv) in N,N-dimethylformamide (30 mL) was stirred overnight at 70° C. under nitrogen atmosphere. The reaction was cooled to room temperature, quenched by the addition of water (30 mL), and extracted by ethyl acetate (3×50 mL). The combined organic layers were washed with brine (30 mL), dried with sodium sulfate, filtered, and concentrated in vacuo. The residue was purified by silica gel column chromatography using a 0-100% gradient of ethyl acetate in petroleum ether to afford methyl 5-bromo-3-[(4-methoxyphenyl)methoxy]pyridine-2-carboxylate (6 g, 67%) as a yellow oil. MS (ESI) calculated for C₁₅H₁₄BrNO₄: 351.01 m/z, found 352.10 [M+H]⁺.

### Step 2: Synthesis of {5-bromo-3-[(4-methoxyphenyl)methoxy]pyridin-2-yl}methanol

To a cooled (−78° C.) solution of methyl 5-bromo-3-[(4-methoxyphenyl)methoxy]pyndine-2-carboxylate (6.00 g, 17.0 mmol, 1 equiv) in tetrahydrofuran (150 mL) was added dropwise diisobutylaluminum hydride (1M in toluene) (51 mL, 51 mmol, 3 equiv). The obtained mixture was stirred at room temperature for 1 h. The reaction was quenched with aqueous potassium sodium tartrate and then methanol (20 mL). Stirring was continued for 10 min and the mixture was extracted with ethyl acetate (100 mL×3). The combined organic extracts were washed with brine (200 mL×2), dried over anhydrous sodium sulfate, filtered, and concentrated in vacuo. The residue was purified by silica gel column chromatography using a 0-100% gradient of ethyl acetate in petroleum ether to afford {5-bromo-3-[(4-methoxyphenyl)methoxy]pyridin-2-yl}methanol (3.5 g, 57%) as a yellow solid. MS (ESI) calculated for C₁₄H₁₄BrNO₃: 323.02 m/z, found 324.10 [M+H]⁺.

### Step 3: Synthesis of 5-bromo-3-[(4-methoxyphenyl)methoxy]pyridine-2-carbaldehyde

To a cooled (0° C.) solution of {5-bromo-3-[(4-methoxyphenyl)methoxy]pyridin-2-yl}methanol (3.50 g, 10.8 mmol, 1 equiv) in dichloromethane (50 mL) was added Dess-Martin periodinane (5.50 g, 13.0 mmol, 1.2 equiv). The mixture was stirred at room temperature for 1 h. The reaction was quenched with water and extracted with ethyl acetate (100 mL×3). The combined organic extracts were washed with brine (100 mL×3), dried over anhydrous sodium sulfate, filtered, and concentrated in vacuo. The residue was purified by silica gel column chromatography to afford 5-bromo-3-[(4-methoxyphenyl)methoxy]pyridine-2-carbaldehyde (1.6 g, 41%) as a yellow solid. MS (ESI) calculated for C₁₄H₁₂BrNO₃: 321.00 m/z, found 320.10 [M−H]⁻.

### Step 4: Synthesis of 5-bromo-2-(1,3-dioxolan-2-yl)-3-((4-methoxybenzyl)oxy)pyridine

A mixture of 5-bromo-3-((4-methoxybenzyl)oxy)picolinaldehyde (2.70 g, 8.38 mmol, 1 equiv), p-toluenesulfonic acid (0.14 g, 0.84 mmol, 0.1 equiv), ethylene glycol (7.09 g, 114 mmol, 5 equiv) and triethylorthoformate (3.73 g, 25.1 mmol, 3 equiv) in toluene (30 mL) was stirred overnight at 90° C. The resulting mixture was concentrated under reduced pressure and purified by silica gel column chromatography using a 0-100% gradient of ethyl acetate in petroleum ether to afford 5-bromo-2-(1,3-dioxolan-2-yl)-3-((4-methoxybenzyl)oxy)pyridine (1.5 g, 48%) as a white solid. MS (ESI) calculated for C₁H₁₆BrNO₄: 365.03 m/z, found 366.20 [M+H]⁺.

### Step 5: Synthesis of ethyl 2-[6-(1,3-dioxolan-2-yl)-5-[(4-methoxyphenyl)methoxy]pyridin-3-yl]acetate

A mixture of 5-bromo-2-(1,3-dioxolan-2-yl)-3-[(4-methoxyphenyl)methoxy]pyridine (1.7 g, 4.6 mmol, 1 equiv), [1,1′-bis(diphenylphosphino)ferrocene]dichloropalladium(II) (0.34 g, 0.46 mmol, 0.1 equiv) and XPhos (0.44 g, 0.93 mmol, 0.2 equiv) in a solution of (2-ethoxy-2-oxoethyl)zinc(II) bromide (45 mL, 193 mmol) in tetrahydrofuran was stirred overnight at 80° C. under nitrogen atmosphere. The mixture was concentrated and purified by reverse phase flash column chromatography on C18 silica gel using a 5-70% gradient of acetonitrile in water (+0.05% ammonium bicarbonate) to afford ethyl 2-[6-(1,3-dioxolan-2-yl)-5-[(4-methoxyphenyl)methoxy]pyridin-3-yl]acetate (350 mg, 19%) as a yellow solid. MS (ESI) calculated for C₂₀H₂₃NO₆, 373.15 m/z, found 374.10 [M+H]⁺.

### Step 6: Synthesis of ethyl 1-[6-(1,3-dioxolan-2-yl)-5-[(4-methoxyphenyl)methoxy]pyridin-3-yl]cyclopropane-1-carboxylate

To a solution of ethyl 2-[6-(1,3-dioxolan-2-yl)-5-[(4-methoxyphenyl)methoxy]pyridin-3-yl]acetate (350 mg, 0.937 mmol, 1 equiv) in dimethyl sulfoxide (5 mL) were added ethenyl diphenylsulfanium triflate (240 mg, 1.12 mmol, 1.2 equiv) and 1,8-diazabicyclo(5.4.0)undec-7-ene (419 mg, 2.81 mmol, 3 equiv). The resulting mixture was stirred for 6 h at room temperature then diluted with water (10 mL) and extracted with ethyl acetate (20 mL×3). The combined organic phases were washed with brine (50 mL×3), dried with sodium sulfate, filtered, and concentrated in vacuo. The residue was purified by silica gel column chromatography using a 0-100% gradient of ethyl acetate in petroleum ether to afford ethyl 1-[6-(1,3-dioxolan-2-yl)-5-[(4-methoxyphenyl)methoxy]pyridin-3-yl]cyclopropane-1-carboxylate (300 mg, 80%) as a white solid. MS (ESI) calculated for C₂₂H₂₅NO₆: 399.17 m/z, found 400.02 [M+H]⁺.

### Step 7: Synthesis of 1-(6-(1,3-dioxolan-2-yl)-5-((4-methoxybenzyl)oxy)pyridin-3-yl) cyclopropane-1-carboxylic acid (Intermediate 56-1)

To a solution of methyl 1-[6-(1,3-dioxolan-2-yl)-5-[(4-methoxyphenyl)methoxy]pyridin-3-yl]cyclopropane-1-carboxylate (300 mg, 0.778 mmol, 1 equiv) in tetrahydrofuran (3 mL) was added a solution of lithium hydroxide (56 mg, 2.3 mmol, 3 equiv) in water (1 mL). The mixture was stirred at room temperature for 0.5 h. The pH was adjusted to ˜6 with 1N hydrochloric acid and the mixture was concentrated in vacuo to afford 1-(6-(1,3-dioxolan-2-yl)-5-((4-methoxybenzyl)oxy)pyridin-3-yl)cyclopropane-1-carboxylic acid (Intermediate 56-1) as a brown solid, which was used without further purification in subsequent transformations. MS (ESI) calculated for C₂₀H₂₁NO₆: 371.14 m/z, found 372.14 [M+H]⁺.

### Example 57: 4-[2-(2-aminopyridin-3-yl)-5-phenylimidazo[4,5-b]pyridin-3-yl]-N-[2-(4-formyl-3-hydroxyphenyl)ethyl]-N-methylbenzamide

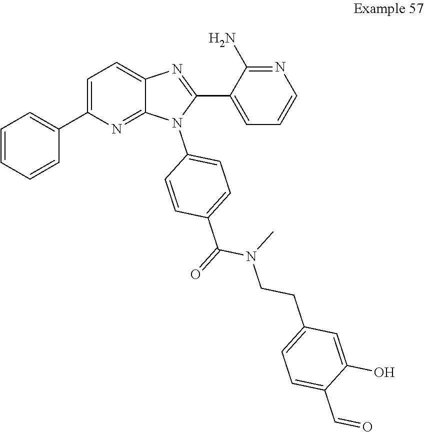 [SMILES: CN(CCc1ccc(C=O)c(O)c1)C(=O)c1ccc(-n2c(-c3cccnc3N)nc3ccc(-c4ccccc4)nc32)cc1]

### Synthetic Route:

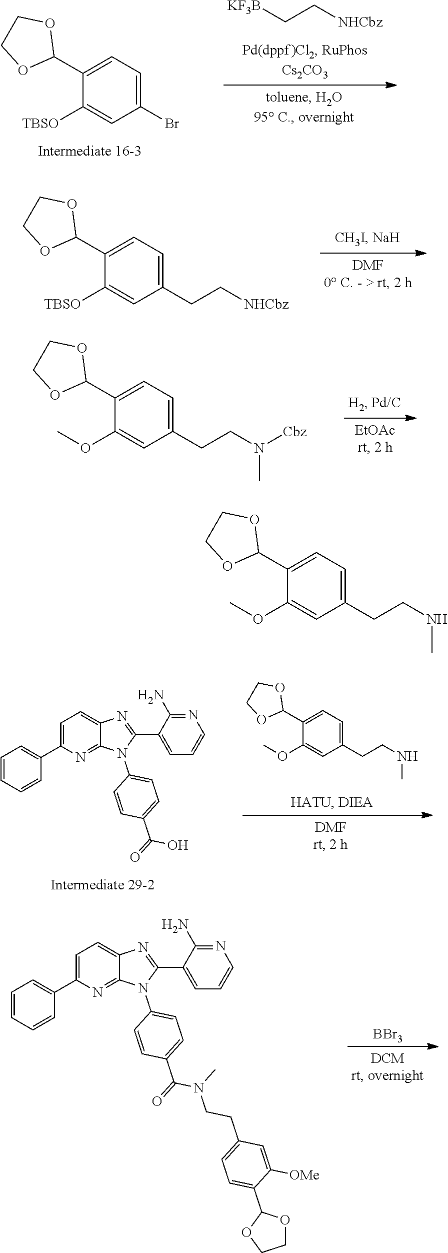

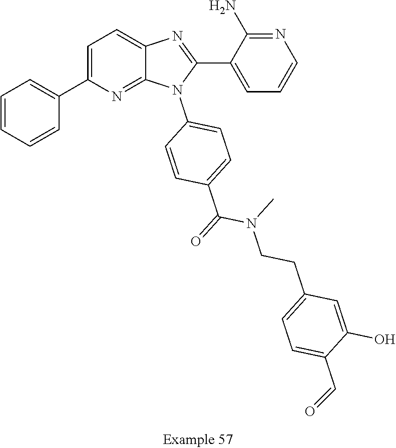 [SMILES: CN(CCc1ccc(C=O)c(O)c1)C(=O)c1ccc(-n2c(-c3cccnc3N)nc3ccc(-c4ccccc4)nc32)cc1]

### Step 1: Synthesis of benzyl N-(2-{3-[(tert-butyldimethylsilyl)oxy]-4-(1,3-dioxolan-2-yl) phenyl}ethyl)carbamate

To a stirred mixture of benzyl N-[2-(trifluoro-lambda4-boranyl)ethyl]carbamate potassium (4.76 g, 16.7 mmol, 3 equiv) in toluene (200 mL) and water (20 mL) were added cesium carbonate (3.63 g, 11.1 mmol, 2 equiv), [1,1′-bis(diphenylphosphino)ferrocene]dichloropalladium(II) (0.41 g, 0.56 mmol, 0.1 equiv), RuPhos (0.26 g, 0.56 mmol, 0.1 equiv) and 5-bromo-2-(1,3-dioxolan-2-yl)phenoxy(tert-butyl)dimethylsilane (Intermediate 16-3) (2.00 g, 5.57 mmol, 1 equiv) and the resulting mixture was stirred overnight at 80° C. under nitrogen atmosphere. The mixture was cooled to room temperature and diluted with water (20 mL). The resulting mixture was extracted with ethyl acetate (3×150 mL). The combined organic layers were washed with brine (100 mL), dried over anhydrous sodium sulfate, filtered, and concentrated under reduced pressure. The residue was purified by silica gel column chromatography using a 0-20% gradient of ethyl acetate in petroleum ether to afford benzyl N-(2-{3-[(tert-butyldimethylsilyl)oxy]-4-(1,3-dioxolan-2-yl)phenyl}ethyl)carbamate (680 mg, 20%) as a yellow viscous oil. MS (ESI) calculated for C₂₅H₃₅NO₅Si: 457.23 m/z, found 458.35 [M+H]⁺.

### Step 2: Synthesis of benzyl (4-(1,3-dioxolan-2-yl)-3-methoxyphenethyl)(methyl)carbamate

To a cooled (0° C.) mixture of benzyl N-(2-{3-[(tert-butyldimethylsilyl)oxy]-4-(1,3-dioxolan-2-yl)phenyl}ethyl)carbamate (680 mg, 1.49 mmol, 1 equiv) in N,N-dimethylformamide (5 mL) was added sodium hydride (75 mg, 3.0 mmol, 2.1 equiv) under nitrogen atmosphere and the obtained mixture was stirred at 0° C. for 30 min. Methyl iodide (422 mg, 1.49 mmol, 2 equiv) was added and the mixture was stirred for 2 hours at room temperature. The reaction was quenched with saturated aqueous ammonium chloride (20 mL) and the mixture was extracted with ethyl acetate (20 mL×3). The combined organic phases were washed with brine (40 mL×3), dried with sodium sulfate, filtered, and concentrated in vacuo. The residue was purified by silica gel column chromatography using a 0-80% gradient of ethyl acetate in petroleum ether to afford benzyl N-{2-[4-(1,3-dioxolan-2-yl)-3-methoxyphenyl]ethyl}-N-methylcarbamate (480 mg, 68%) as a light-yellow oil. MS (ESI) calculated for C₂₁H₂₅NO₅: 371.17 m/z, found 372.25 [M+H]⁺.

### Step 3: Synthesis of {2-[4-(1,3-dioxolan-2-yl)-3-methoxyphenyl]ethyl}(methyl)amine

A solution of benzyl (4-(1,3-dioxolan-2-yl)-3-methoxyphenethyl)(methyl)carbamate (480 mg, 1.29 mmol, 1 equiv) in ethyl acetate (30 mL) was added 10% palladium on carbon (240 mg, 0.2 equiv). The resulting mixture was stirred overnight at room temperature under hydrogen atmosphere. The mixture was diluted with dichloromethane (50 mL) and filtered, rinsing with dichloromethane (3×50 mL). The filtrate was concentrated under reduced pressure to afford {2-[4-(1,3-dioxolan-2-yl)-3-methoxyphenyl]ethyl}(methyl)amine (240 mg, 59%) as a yellow oil. MS (ESI) calculated for C₁₃H₁₉NO₃: 237.14 m/z, found 238.20 [M+H]⁺.

### Step 4: Synthesis of 4-[2-(2-aminopyridin-3-yl)-5-phenylimidazo[4,5-b]pyridin-3-yl]-N-[2-(4-formyl-3-hydroxyphenyl)ethyl]-N-methylbenzamide

To a solution of 4-[2-(2-aminopyridin-3-yl)-5-phenylimidazo[4,5-b]pyridin-3-yl]benzoic acid (Intermediate 29-2) (200 mg, 0.491 mmol, 1 equiv) and N,N-diisopropylethylamine (127 mg, 0.982 mmol, 2 equiv) in N,N-dimethylformamide (4 mL) were added HATU (187 mg, 0.491 mmol, 1 equiv) and {2-[4-(1,3-dioxolan-2-yl)-3-methoxyphenyl]ethyl}(methyl)amine (116 mg, 0.491 mmol, 1 equiv). The reaction mixture was stirred for 2 h at room temperature. The mixture was purified by reverse-phase column chromatography on C18 silica gel using a 5-70% gradient of acetonitrile in water (+0.05% 2,2,2-trifluoroacetic acid) to afford 4-[2-(2-aminopyridin-3-yl)-5-phenylimidazo[4,5-b]pyridin-3-yl]-N-{2-[4-(1,3-dioxolan-2-yl)-3-methoxyphenyl-]ethyl}-N-methylbenzamide (60 mg, 17%) as a yellow solid. MS (ESI) calculated for C₃₀H₂₁N₅O₂: 626.26 m/z, found 627.45 [M+H]⁺.

### Step 5: Synthesis of 4-[2-(2-aminopyridin-3-yl)-5-phenylimidazo[4,5-b]pyridin-3-yl]-N-[2-(4-formyl-3-hydroxyphenyl)ethyl]-N-methylbenzamide (Example 57)

To a stirred mixture of 4-[2-(2-aminopyridin-3-yl)-5-phenylimidazo[4,5-b]pyridin-3-yl]-N-{2-[4-(1,3-dioxolan-2-yl)-3-methoxyphenyl]ethyl}-N-methylbenzamide (60 mg, 0.096 mmol, 1 equiv) in dichloromethane (5 mL) was added boron tribromide (0.96 mL, 0.96 mmol, 10 equiv) dropwise at −30° C. under nitrogen atmosphere. The resulting mixture was stirred overnight at room temperature under nitrogen atmosphere. The mixture was quenched with saturated aqueous sodium bicarbonate until the pH was ˜6. The resulting mixture was extracted with ethyl acetate (6×30 mL). The combined organic layers were dried over anhydrous sodium sulfate, filtered, and concentrated in vacuo. The residue was purified by preparative HPLC on a XBridge Shield RP18 OBD Column using a 47-62% gradient of acetonitrile in water (+10 mmol/L ammonium bicarbonate) to afford 4-[2-(2-aminopyridin-3-yl)-5-phenylimidazo[4,5-b]pyridin-3-yl]-N-[2-(4-formyl-3-hydroxyphenyl)ethyl]-N-methylbenzamide (Example 57) (8.4 mg, 15%) as a yellow solid. MS (ESI) calculated for C₃₄H₂₈N₆O₃: 568.22 m/z, found 569.10 [M+H]⁺. ¹H NMR (300 MHz, DMSO-d₆) δ (ppm): 10.13-10.19 (s, 1H), 8.28-8.30 (m, 1H), 8.00-8.07 (m, 4H), 7.43-7.66 (m, 7H), 7.30-7.33 (m, 2H), 6.95-6.60 (m, 1H), 6.46-6.63 (m, 1H), 6.33-6.44 (m, 1H), 3.51-3.71 (m, 1H), 3.40-3.45 (m, 1H), 2.90 (s, 2H), 2.74-2.83 (m, 2H), 2.51-2.73 (m, 1H).

### Example 58: 2-{4-[2-(2-aminopyridin-3-yl)-5-phenylimidazo[4,5-b]pyridin-3-yl]phenyl}-N-[(4-formyl-3-hydroxyphenyl)methyl]acetamide

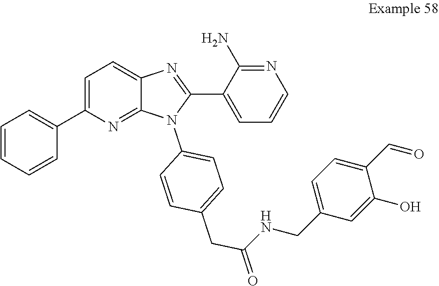 [SMILES: Nc1ncccc1-c1nc2ccc(-c3ccccc3)nc2n1-c1ccc(CC(=O)NCc2ccc(C=O)c(O)c2)cc1]

Example 58 was prepared in a manner analogous to Example 29 using Intermediate 11-1 in place of Intermediate 29-2 and 1-(1-benzofuran-6-yl)methanamine in place of Intermediate 29-3. MS (ESI) calculated for C₃₃H₂₆N₆O₃, 554.21 m/z: found 555.20 [M+H]⁺. ¹H NMR (300 MHz, DMSO-d₆) δ (ppm): 10.16 (s, 1H), 8.26-8.28 (m, 1H), 7.97-8.04 (m, 4H), 7.59-7.61 (m, 1H), 7.40-7.45 (m, 7H), 7.20-7.22 (m, 1H), 6.80-6.88 (m, 2H), 6.38-6.42 (m, 1H), 4.30 (s, 2H), 3.71 (s, 2H).

### Example 59: N-(4-(2-(2-aminopyridin-3-yl)-5-phenyl-3H-imidazo[4,5-b]pyridin-3-yl) benzyl)-1-(2-fluoro-4-formyl-3-hydroxyphenyl)cyclopropane-1-carboxamide

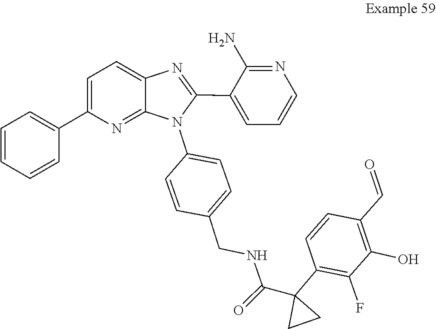 [SMILES: Nc1ncccc1-c1nc2ccc(-c3ccccc3)nc2n1-c1ccc(CNC(=O)C2(c3ccc(C=O)c(O)c3F)CC2)cc1]

Example 59 was prepared in a manner analogous to Example 1 (starting from Step 3) using Intermediate 4-1 in place of the amine starting material and Intermediate 59-1 in place of Intermediate 1-4. MS (ESI) calculated for C₃₅H₂₇FN₆O₃: 598.21 m/z, found 599.20 [M+H]⁺. ¹H NMR (300 MHz, DMSO-d₆) δ (ppm): 10.19 (s, 1H), 8.30-8.45 (m, 1H), 8.00-8.19 (m, 4H), 7.72-7.90 (m, 1H), 7.40-7.60 (m, 6H), 7.30-7.40 (m, 2H), 6.90-7.15 (m, 1H), 6.65-6.90 (m, 1H), 4.20-4.50 (m, 2H), 1.40-1.60 (m, 2H), 1.00-1.30 (m, 2H). ¹⁹F NMR (282 MHz, DMSO-d₆) δ (ppm): −135.97.

### Intermediate 59-1: 1-(4-(1,3-dioxolan-2-yl)-2-fluoro-3-((4-methoxybenzyl)oxy)phenyl) cyclopropane-1-carboxylic acid

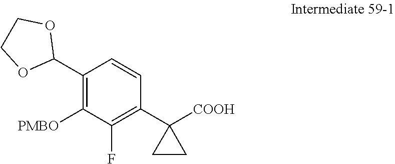

### Synthetic Route:

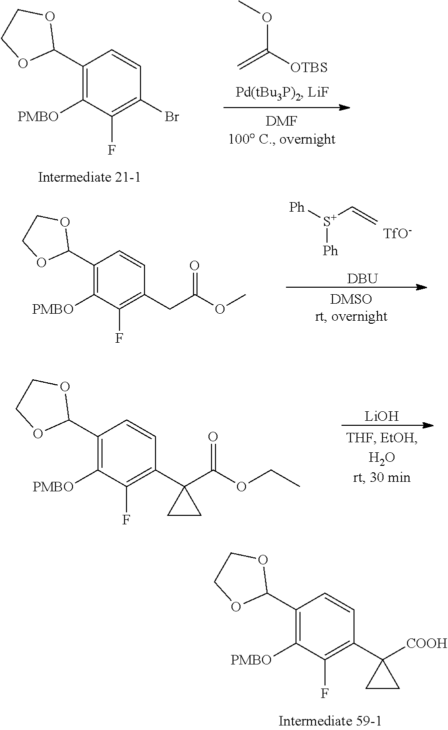

### Step 1: Synthesis of methyl 2-[4-(1,3-dioxolan-2-yl)-2-fluoro-3-[(4-methoxyphenyl) methoxy]phenyl]acetate

To a stirred solution of 2-{4-bromo-3-fluoro-2-[(4-methoxyphenyl)methoxy]phenyl}-1,3-dioxolane (Intermediate 21-1) (3.00 g, 7.83 mmol, 1 equiv) and tert-butyl[(1-methoxyethenyl)oxy]dimethylsilane (6.00 g, 31.9 mmol, 4 equiv) in N,N-dimethylformamide (12 mL) were added lithium fluoride (450 mg, 17.3 mmol, 2.22 equiv) and bis(tri-tert-butylphosphine)palladium(0) (450 mg, 0.881 mmol, 0.11 equiv) at room temperature under nitrogen atmosphere. The resulting mixture was stirred overnight at 100° C. The reaction was quenched by the addition of water (10 mL) at room temperature. The resulting mixture was extracted with ethyl acetate (3×10 mL). The combined organic layers were washed with brine (3×10 mL), dried over anhydrous sodium sulfate, filtered, and concentrated under reduced pressure. The residue was purified by silica gel column chromatography using a 0-20% gradient of ethyl acetate in petroleum ether to afford methyl 2-[4-(1,3-dioxolan-2-yl)-2-fluoro-3-[(4-methoxyphenyl)methoxy]phenyl]acetate (2 g, 68%) as a white solid. MS (ESI) calculated for C₂₀H₂₁FO₆: 376.13 m/z, found 377.10 [M+H]⁺.

### Step 2: Synthesis of methyl 1-[4-(1,3-dioxolan-2-yl)-2-fluoro-3-[(4-methoxyphenyl) methoxy]phenyl]cyclopropane-1-carboxylate

A mixture of methyl 2-[4-(1,3-dioxolan-2-yl)-2-fluoro-3-[(4-methoxyphenyl) methoxy]phenyl]acetate (1.5 g, 3.985 mmol, 1 equiv), ethenyldiphenylsulfanium triflate (1.8 g, 5.0 mmol, 1.25 equiv) and 1,8-diazabicyclo(5.4.0)undec-7-ene (1.95 g, 12.8 mmol, 3.2 equiv) in dimethyl sulfoxide (75 mL) was stirred overnight at room temperature. The reaction was quenched by the addition of water (100 mL) and the resulting mixture was extracted with ethyl acetate (3×100 mL). The combined organic layers were washed with brine (3×100 mL), dried over anhydrous sodium sulfate, filtered, and concentrated under reduced pressure. The residue was purified by silica gel column chromatography using a 0-15% gradient of ethyl acetate in petroleum ether to afford methyl 1-[4-(1,3-dioxolan-2-yl)-2-fluoro-3-[(4-methoxyphenyl)methoxy]phenyl]cyclopropane-1-carboxylate (1 g, 62%) as a yellow oil. MS (ESI) calculated for C₂₂H₂₃FO₆: 402.15 m/z, found 403.10 [M+H]⁺.

### Step 3: Synthesis of 1-[4-(1,3-dioxolan-2-yl)-2-fluoro-3-[(4-methoxyphenyl)methoxy]phenyl]cyclopropane-1-carboxylic acid (Intermediate 59-1)

A mixture of methyl 1-[4-(1,3-dioxolan-2-yl)-2-fluoro-3-[(4-methoxyphenyl)methoxy]phenyl]cyclopropane-1-carboxylate (900 mg, 2.24 mmol, 1 equiv) and lithium hydroxide (180 mg, 7.52 mmol, 3.36 equiv) in tetrahydrofuran (3 mL), water (3 mL) and ethanol (3 mL) was stirred for 2 h at room temperature. The resulting mixture was concentrated under reduced pressure. The mixture was then diluted with water (5 mL) and the pH was brought to 6 with 1N hydrochloric acid. The resulting mixture was extracted with ethyl acetate (3×5 mL). The combined organic layers were washed with brine (3×10 mL), dried over anhydrous sodium sulfate, filtered, and concentrated under reduced pressure to afford 1-[4-(1,3-dioxolan-2-yl)-2-fluoro-3-[(4-methoxyphenyl)methoxy]phenyl]cyclopropane-1-carboxylic acid (Intermediate 59-1) (900 mg, crude quant.) as a white solid, which was used in subsequent transformations without further purification. MS (ESI) calculated for C₂₁H₂₁FO₆: 388.13 m/z, found 411.15 [M+Na]⁺.

### Example 60: N-({4-[2-(2-aminopyridin-3-yl)-5-phenylimidazo[4,5-b]pyridin-3-yl]phenyl}methyl)-2-(4-formyl-3-hydroxyphenyl)-2-methylpropanamide

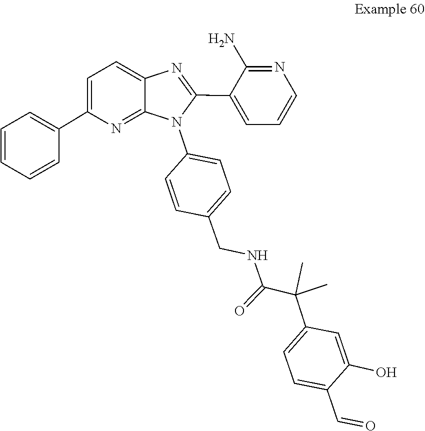 [SMILES: CC(C)(C(=O)NCc1ccc(-n2c(-c3cccnc3N)nc3ccc(-c4ccccc4)nc32)cc1)c1ccc(C=O)c(O)c1]

Example 60 was prepared in a manner analogous to Example 1 (starting from Step 4) using Intermediate 4-1 in place of the amine starting material and Intermediate 60-1 in place of Intermediate 1-4. MS (ESI) calculated for C₃₅H₃₀N₆O₃, 582.24 m/z: found 583.25 [M+H]⁺. ¹H NMR (300 MHz, DMSO-d₆) δ (ppm): 10.20 (s, 1H), 8.25-8.30 (m, 1H), 8.10-8.15 (m, 1H), 7.95-8.05 (m, 4H), 7.55-7.65 (m, 1H), 7.45-7.50 (m, 2H), 7.35-7.45 (m, 3H), 7.25-7.35 (m, 2H), 7.15-7.25 (m, 1H), 6.90-7.05 (m, 4H), 6.37-6.45 (m, 1H), 4.35-4.37 (m, 2H), 1.50 (m, 6H).

### Intermediate 60-1: 2-(4-(1,3-dioxolan-2-yl)-3-((4-methoxybenzyl)oxy)phenyl)-2-methylpropanoic acid

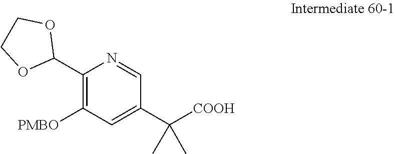

### Synthetic Route:

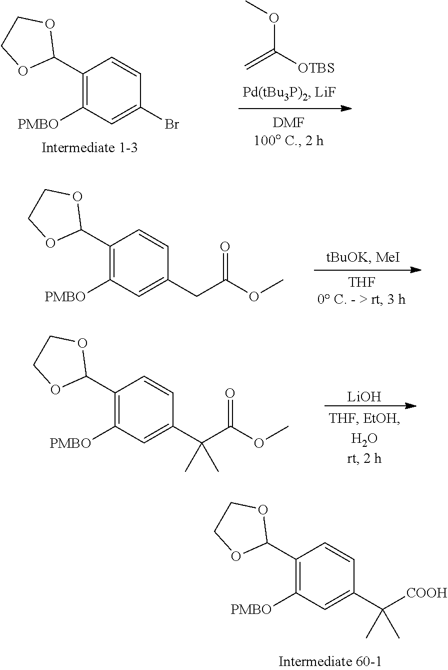

### Step 1: Synthesis of methyl 2-(4-(1,3-dioxolan-2-yl)-3-((4-methoxybenzyl)oxy)phenyl) acetate

To a solution of 2-(4-bromo-2-((4-methoxybenzyl)oxy)phenyl)-1,3-dioxolane (Intermediate 1-3) (4.15 g, 11.3 mmol, 1 equiv) and tert-butyldimethylsilyl methyl carbonate (8.65 g, 45.4 mmol, 4 equiv) in N,N-dimethylformamide (20 mL) were added bis(tri-tert-butylphosphine)palladium(0) (0.60 g, 1.14 mmol, 0.1 equiv) and lithium fluoride (0.6 g, 23 mmol, 2 equiv). The resulting mixture was stirred at 100° C. under nitrogen atmosphere for 2 h then cooled to 0° C. and quenched with water (100 mL). The resulting mixture was extracted with ethyl acetate (200 mL×3). The combined organic layers were washed with water (100 mL×3), dried over anhydrous sodium sulfate, filtered, and concentrated in vacuo. The residue was purified by silica gel column chromatography using a 0-60% gradient of ethyl acetate in petroleum ether to provide methyl 2-(4-(1,3-dioxolan-2-yl)-3-((4-methoxybenzyl)oxy) phenyl)acetate (3.0 g, 74%) as a light-yellow oil. MS (ESI) calculated for C₂₀H₂₂O₆: 358.14 m/z, found 359.10 [M+H]⁺.

### Step 2: Synthesis of methyl 2-[4-(1,3-dioxolan-2-yl)-3-[(4-methoxyphenyl)methoxy]phenyl]-2-methylpropanoate

To a cooled (0° C.) solution of methyl 2-[4-(1,3-dioxolan-2-yl)-3-[(4-methoxyphenyl) methoxy]phenyl]acetate (1.00 g, 2.79 mmol, 1 equiv) in tetrahydrofuran (5 mL) were added dropwise a solution of potassium tert-butoxide (9 mL, 9.00 mmol, 3.2 equiv, 1M in tetrahydrofuran) and methyl iodide (1.66 g, 11.7 mmol, 4.2 equiv) with stirring. The resulting mixture was stirred at room temperature for 2 h. The reaction was quenched by addition of 1M aqueous potassium bisulfate, and the mixture was extracted with ethyl acetate. The organic extract was washed with brine, dried over anhydrous sodium sulfate, filtered, and concentrated under reduced pressure to afford methyl 2-[4-(1,3-dioxolan-2-yl)-3-[(4-methoxyphenyl) methoxy]phenyl]-2-methylpropanoate (680 mg, 63%) as a yellow solid, which was used without purification in the next step. MS (ESI) calculated for C₂₂H₂₆O₆: 386.17 m/z, found 387.15 [M+H]⁺.

### Step 3: Synthesis of 2-[4-(1,3-dioxolan-2-yl)-3-[(4-methoxyphenyl)methoxy]phenyl]-2-methylpropanoic acid (Intermediate 60-1)

To a stirred solution of methyl 2-[4-(1,3-dioxolan-2-yl)-3-[(4-methoxyphenyl) methoxy]phenyl]-2-methylpropanoate (600 mg, 1.55 mmol, 1 equiv) in tetrahydrofuran (10 mL) and methanol (10 mL) was added a solution of lithium hydroxide (74 mg, 3.1 mmol, 2 equiv) in water (10 mL). The resulting mixture was stirred for 2 h at room temperature. The mixture was acidified to pH 5-6 with hydrochloric acid (1M). The resulting mixture was extracted with ethyl acetate (3×50 mL) and the combined organic layers were washed with brine (50 mL), dried over anhydrous sodium sulfate, filtered, and concentrated under reduced pressure to afford 2-[4-(1,3-dioxolan-2-yl)-3-[(4-methoxyphenyl)methoxy]phenyl]-2-methylpropanoic acid (Intermediate 60-1) (500 mg, 86%) as a brown oil. MS (ESI) calculated for C₂₁H₂₄O₆: 372.16 m/z, found 373.15 [M+H]⁺.

### Example 61: N-({4-[2-(2-aminopyridin-3-yl)-5-(pyridin-4-yl)imidazo[4,5-b]pyridin-3-yl]phenyl}methyl)-2-(4-formyl-3-hydroxyphenyl)acetamide

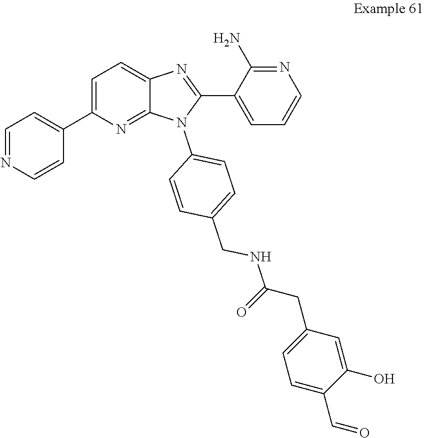 [SMILES: Nc1ncccc1-c1nc2ccc(-c3ccncc3)nc2n1-c1ccc(CNC(=O)Cc2ccc(C=O)c(O)c2)cc1]

Example 61 was prepared in a manner analogous to Example 1 using pyridin-4-ylboronic acid in place of 4-(morpholin-4-yl)phenylboronic acid. MS (ESI) calculated for C₃₂H₂₅N₇O₃: 555.20 m/z, found 556.00 [M+H]⁺. ¹H NMR (400 MHz, DMSO-d₆) δ (ppm): 10.22 (s, 1H), 8.78-8.95 (m, 2H), 8.45-8.52 (m, 1H), 8.30-8.45 (m, 3H), 8.02-8.12 (m, 1H), 7.68-7.80 (m, 1H), 7.58-7.68 (m, 1H), 7.48-7.58 (m, 2H), 7.38-7.48 (m, 2H), 6.82-7.02 (m, 2H), 6.65-6.82 (m, 1H), 4.40 (s, 2H), 3.58 (s, 2H).

### Example 62: N-({4-[2-(2-aminopyridin-3-yl)-5-(pyridin-2-yl)imidazo[4,5-b]pyridin-3-yl]phenyl}methyl)-2-(4-formyl-3-hydroxyphenyl)acetamide

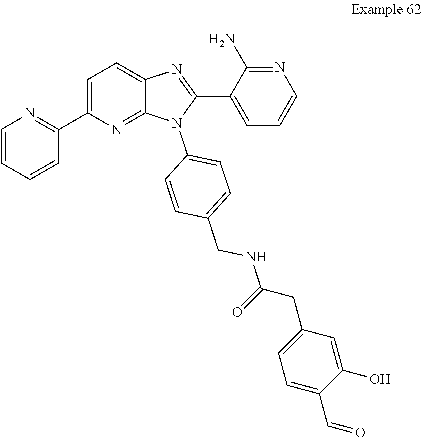 [SMILES: Nc1ncccc1-c1nc2ccc(-c3ccccn3)nc2n1-c1ccc(CNC(=O)Cc2ccc(C=O)c(O)c2)cc1]

Example 62 was prepared in a manner analogous to Example 1 (starting from Step 2) using Intermediate 62-2 in place of the Boc-protected starting material. MS (ESI) calculated for C₃₂H₂₅N₇O₃: 555.20 m/z, found 556.00 [M+H]⁺. ¹H NMR (400 MHz, DMSO-d₆) δ (ppm): 10.22 (s, 1H), 8.68-8.78 (m, 1H), 8.48-8.62 (m, 1H), 8.35-8.48 (m, 1H), 8.15-8.28 (m, 1H), 8.05-8.15 (m, 1H), 7.88-8.05 (m, 1H), 7.75-7.88 (m, 1H), 7.58-7.68 (m, 1H), 7.50-7.58 (m, 2H), 7.28-7.50 (m, 3H), 6.80-7.00 (m, 3H), 4.40 (s, 2H), 3.58 (s, 2H).

### Intermediate 62-1: tert-butyl (4-(2-(2-aminopyridin-3-yl)-5-(pyridin-2-yl)-3H-imidazo[4,5-b]pyridin-3-yl)benzyl)carbamate

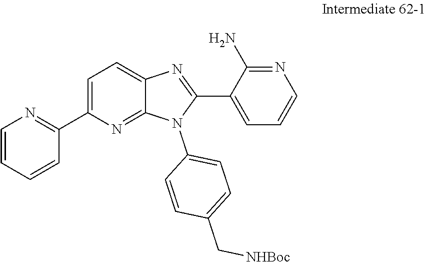 [SMILES: CC(C)(C)OC(=O)NCc1ccc(-n2c(-c3cccnc3N)nc3ccc(-c4ccccn4)nc32)cc1]

### Synthetic Route:

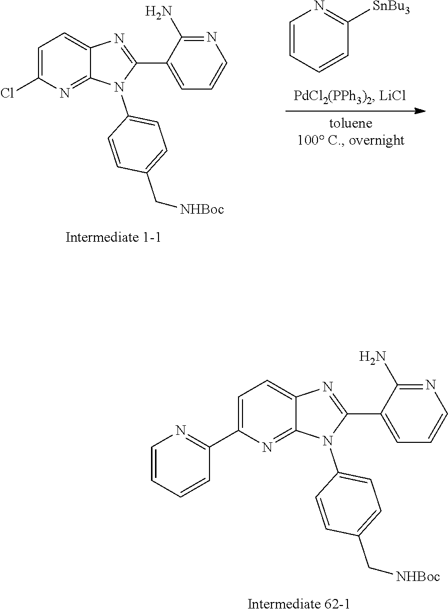

### Step 1: Synthesis of tert-butyl N-({4-[2-(2-aminopyridin-3-yl)-5-(pyridin-2-yl)imidazo[4,5-b]pyridin-3-yl]phenyl}methyl)carbamate (Intermediate 62-1)

To a solution of tert-butyl N-({4-[2-(2-aminopyridin-3-yl)-5-chloroimidazo[4,5-b]pyridin-3-yl]phenyl}methyl)carbamate (Intermediate 1-1) (500 mg, 1.11 mmol, 1 equiv) and 2-(tributylstannyl)pyridine (612 mg, 1.66 mmol, 1.5 equiv) in toluene (5 mL) were added lithium chloride (188 mg, 4.44 mmol, 4 equiv) and bis(triphenylphosphine)palladium(II) dichloride (78 mg, 0.11 mmol, 0.1 equiv). The resulting mixture was stirred overnight at 100° C. under nitrogen atmosphere then cooled to room temperature and concentrated under reduced pressure. The residue was purified by reverse-phase flash column chromatography on C18 silica gel using a 10-50% gradient of acetonitrile in water (+0.05% ammonium bicarbonate) to afford tert-butyl N-({4-[2-(2-aminopyridin-3-yl)-5-(pyridin-2-yl)imidazo[4,5-b]pyridin-3-yl]phenyl}methyl)carbamate (Intermediate 62-1) (300 mg, 53%) as a brown solid. MS (ESI) calculated for C₂₈H₂₇N₇O₂: 493.22 m/z, found 494.25 [M+H]⁺.

### Example 63: N-({4-[2-(2-aminopyridin-3-yl)-5-phenylimidazo[4,5-b]pyridin-3-yl]phenyl}methyl)-2-(2-chloro-4-formyl-3-hydroxyphenyl)acetamide

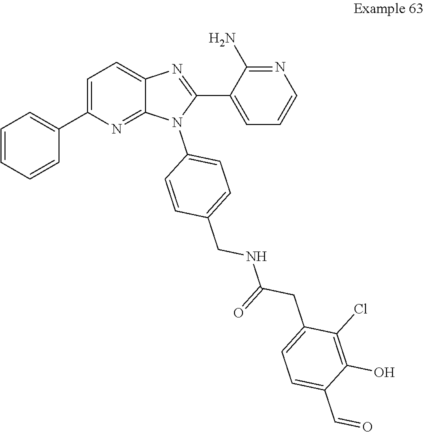 [SMILES: Nc1ncccc1-c1nc2ccc(-c3ccccc3)nc2n1-c1ccc(CNC(=O)Cc2ccc(C=O)c(O)c2Cl)cc1]

Example 63 was prepared in a manner analogous to Example 1 (starting from Step 3) using Intermediate 4-1 in place of the amine starting material and Intermediate 13-2 in place of Intermediate 1-4. MS (ESI) calculated for C₃₃H₂₅ClN₆O₃: 588.17 m/z, found 589.20 [M+H]⁺. ¹H NMR (400 MHz, DMSO-d₆) δ (ppm): 10.08 (s, 1H), 8.74 (s, 1H), 8.26-8.28 (m, 1H), 7.98-8.02 (m, 4H), 7.67-7.69 (m, 1H), 7.40-7.49 (m, 7H), 7.11-7.23 (m, 2H), 6.41-6.44 (m, 1H), 4.41-4.43 (m, 2H), 3.79-3.80 (m, 2H).

### Example 64: N-(4-(2-(2-aminopyridin-3-yl)-5-phenyl-3H-imidazo[4,5-b]pyridin-3-yl)benzyl)-2-(4-formyl-3-hydroxyphenyl)-N-methylacetamide

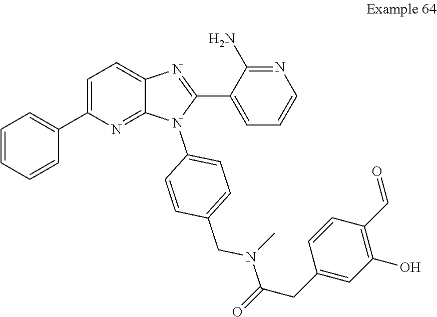 [SMILES: CN(Cc1ccc(-n2c(-c3cccnc3N)nc3ccc(-c4ccccc4)nc32)cc1)C(=O)Cc1ccc(C=O)c(O)c1]

Example 64 was prepared in a manner analogous to Example 1 (starting from Step 3) using Intermediate 64-1 in place of the amine starting material. MS (ESI) calculated for C₃₄H₂₈N₆O₃: 568.22 m/z, found 569.20 [M+H]⁺. ¹H NMR (400 MHz, DMSO-d₆) δ (ppm) 10.23 (s, 1H), 8.30-8.41 (m, 1H), 8.01-8.11 (m, 4H), 7.63-7.70 (m, 1H), 7.60-7.70 (m, 1H), 7.40-7.62 (m, 7H), 6.91-7.01 (m, 2H), 6.75-6.90 (m, 1H), 4.61-4.80 (m, 2H), 3.81-3.98 (m, 2H), 3.10-2.85 (m, 3H).

### Intermediate 64-1: 3-(3-(4-((methylamino)methyl)phenyl)-5-phenyl-3H-imidazo[4,5-b]pyridin-2-yl)pyridin-2-amine

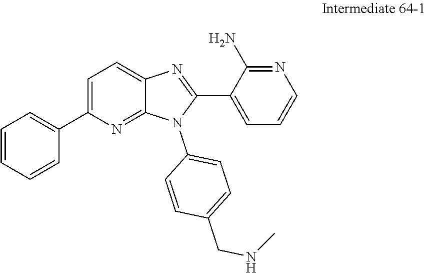 [SMILES: CNCc1ccc(-n2c(-c3cccnc3N)nc3ccc(-c4ccccc4)nc32)cc1]

Intermediate 64-1 was prepared in a manner analogous to Intermediate 11-1 using tert-butyl N-[(4-aminophenyl)methyl]-N-methylcarbamate in place of tert-butyl 2-(4-aminophenyl)acetate. MS (ESI) calculated for C₂₅H₂₂N₆: 406.19 m/z, found 407.19 [M+H]⁺.

### Example 65: N-({4-[2-(2-aminopyridin-3-yl)-5-phenylimidazo[4,5-b]pyridin-3-yl]phenyl}methyl)-2-fluoro-2-(4-formyl-3-hydroxyphenyl)acetamide

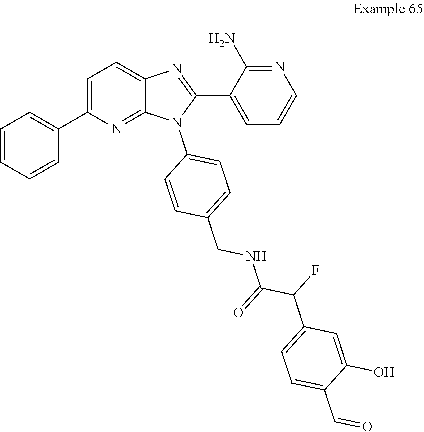 [SMILES: Nc1ncccc1-c1nc2ccc(-c3ccccc3)nc2n1-c1ccc(CNC(=O)C(F)c2ccc(C=O)c(O)c2)cc1]

Example 65 was prepared in a manner analogous to Example 1 (starting from Step 3) using Intermediate 4-1 in place of the amine starting material and Intermediate 65-1 in place of Intermediate 1-4. MS (ESI) calculated for C₃₃H₂₅FN₆O₃: 572.20 m/z, found 573.10 [M+H]⁺. ¹H NMR (400 MHz, DMSO-d₆) δ (ppm): 10.25 (s, 1H), 9.22-9.26 (m, 1H), 8.32-8.35 (m, 1H), 8.01-8.06 (m, 4H), 7.70-7.75 (m, 2H), 7.39-7.52 (m, 7H), 7.07-7.09 (m, 1H), 6.80-7.06 (m, 1H), 6.75-6.78 (m, 1H), 5.96-6.12 (m, 1H), 4.44-4.46 (m, 2H). ¹⁹F NMR (300 MHz, DMSO-d₆) δ (ppm): −181.34.

### Intermediate 65-1: 2-(4-(1,3-dioxolan-2-yl)-3-((4-methoxybenzyl)oxy)phenyl)-2-fluoroacetic acid

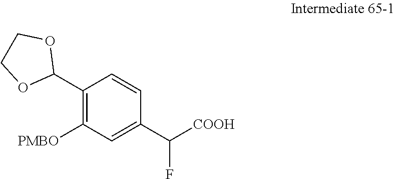

### Synthetic Route:

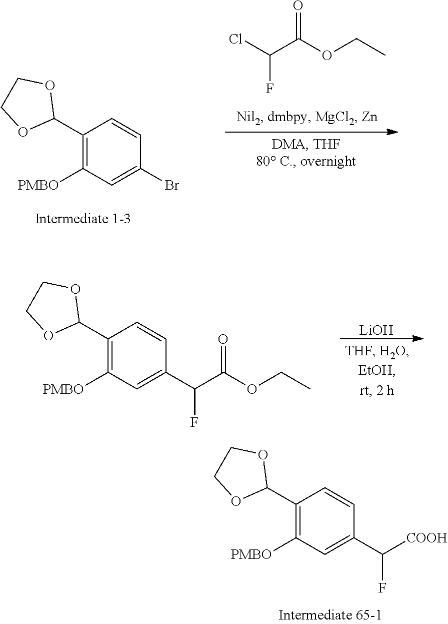

### Step 1: Synthesis of ethyl 2-[4-(1,3-dioxolan-2-yl)-3-[(4-methoxyphenyl)methoxy]phenyl]-2-fluoroacetate

A mixture of 2-{4-bromo-2-[(4-methoxyphenyl)methoxy]phenyl}-1,3-dioxolane (Intermediate 1-3) (1.00 g, 2.74 mmol, 1 equiv), ethyl 2-chloro-2-fluoroacetate (0.77 g, 5.5 mmol, 2 equiv), nickel(II) iodide (0.086 g, 0.28 mmol, 0.10 equiv), 5,5′-dimethyl-2,2′-bipyridine (0.030 g, 0.16 mmol, 0.06 equiv), BINAP (0.086 g, 0.14 mmol, 0.05 equiv), magnesium(II) chloride (0.39 g, 4.1 mmol, 1.5 equiv) and zinc (0.54 g, 8.2 mmol, 3 equiv) in N,N-dimethylacetamide (20 mL) and tetrahydrofuran (8 mL) was stirred overnight at 80° C. The reaction was quenched by the addition of water (30 mL) at room temperature. The resulting mixture was extracted with ethyl acetate (50 mL×3). The combined organic layers were washed with water (50 mL×3), dried over anhydrous sodium sulfate, filtered, and concentrated under reduced pressure. The residue was purified by silica gel column chromatography using a 0-25% gradient of ethyl acetate in petroleum ether to afford ethyl 2-[4-(1,3-dioxolan-2-yl)-3-[(4-methoxyphenyl)methoxy]phenyl]-2-fluoroacetate (460 mg, 40%) as a yellow oil. ¹H-NMR (400 MHz, DMSO-d₆) δ (ppm): 7.50-7.58 (m, 1H), 7.35-7.48 (m, 2H), 7.12-7.21 (m, 1H), 6.99-7.08 (m, 1H), 6.90-6.98 (m, 2H), 6.01-6.21 (m, 1H), 5.69-5.79 (m, 1H), 5.02-5.19 (m, 2H), 4.26-4.29 (m, 2H), 4.01-4.09 (m, 2H), 3.89-3.99 (m, 2H), 3.70-3.75 (m, 3H), 1.12-1.23 (m, 3H). ¹⁹F-NMR (300 MHz, DMSO-d₆) δ (ppm): −176.55.

### Step 2: Synthesis of [4-(1,3-dioxolan-2-yl)-3-[(4-methoxyphenyl)methoxy]phenyl](fluoro)acetic acid (Intermediate 65-1)

A solution of ethyl 2-[4-(1,3-dioxolan-2-yl)-3-[(4-methoxyphenyl)methoxy]phenyl]-2-fluoroacetate (700 mg, 1.79 mmol, 1 equiv) and lithium hydroxide (86 mg, 3.6 mmol, 2 equiv) in ethanol (10 mL), water (10 mL) and tetrahydrofuran (10 mL) was stirred for 2 h at room temperature. The mixture was partially concentrated in vacuo and diluted with water (30 mL). The mixture was acidified to pH 5-6 with hydrochloric acid (0.05 M) and extracted with ethyl acetate (50 mL×3). The combined organic layers were washed with water (50 mL×3), dried over anhydrous sodium sulfate, filtered, and concentrated under reduced pressure to afford [4-(1,3-dioxolan-2-yl)-3-[(4-methoxyphenyl)methoxy]phenyl](fluoro)acetic acid (Intermediate 65-1) (450 mg, 69%) as a yellow oil, which was used in subsequent transformations without further purification. MS (ESI) calculated for C₁₉H₁₉FO₆: 362.12 m/z, found 361.00 [M−H]⁺.

### Example 66: N-({4-[2-(2-aminopyridin-3-yl)-5-cyclopropylimidazo[4,5-b]pyridin-3-yl]phenyl}methyl)-2-(4-formyl-3-hydroxyphenyl)acetamide

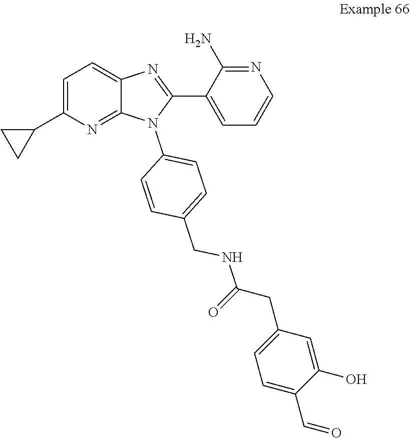 [SMILES: Nc1ncccc1-c1nc2ccc(C3CC3)nc2n1-c1ccc(CNC(=O)Cc2ccc(C=O)c(O)c2)cc1]

Example 66 was prepared in a manner analogous to Example 1 (starting from Step 3) using Intermediate 16-2 in place of the amine starting material. MS (ESI) calculated for C₃₀H₂₆N₆O₃: 518.21 m/z, found 519.25 [M+H]⁺. ¹H-NMR (400 MHz, DMSO-d₆) δ (ppm): 10.18 (s, 1H), 8.08-8.11 (m, 1H), 8.02-8.04 (m, 1H), 7.65-7.67 (m, 1H), 7.66-7.69 (m, 1H), 7.61-7.64 (m, 4H), 7.31-7.41 (m, 1H), 6.93-6.96 (m, 1H), 6.90-6.92 (m, 1H), 6.74-6.79 (m, 1H), 4.37 (s, 2H), 3.65 (s, 2H), 2.18-2.21 (m, 1H), 0.94-1.00 (m, 2H), 0.82-0.83 (m, 2H).

### Example 67: N-(4-(2-(2-aminopyridin-3-yl)-5-(pyridin-3-yl)-3H-imidazo[4,5-b]pyridin-3-yl)benzyl)-1-(2-fluoro-4-formyl-3-hydroxyphenyl)cyclopropane-1-carboxamide

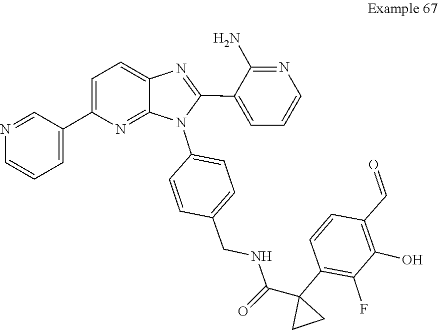 [SMILES: Nc1ncccc1-c1nc2ccc(-c3cccnc3)nc2n1-c1ccc(CNC(=O)C2(c3ccc(C=O)c(O)c3F)CC2)cc1]

Example 67 was prepared in a manner analogous to Example 1 (starting from Step 3) using Intermediate 67-1 in place of the amine starting material and Intermediate 59-1 in place of Intermediate 1-4. MS (ESI) calculated for C₃₄H₂₆FN₇O₃: 599.21 m/z, found 600.20 [M+H]⁺. ¹H NMR (300 MHz, DMSO-d₆) δ (ppm): 10.22 (s, 1H), 9.20-9.30 (m, 1H), 8.65-8.85 (m, 2H), 8.33-8.55 (m, 1H), 8.19-8.30 (m, 1H), 8.00-8.19 (m, 1H), 7.73-7.94 (m, 2H), 7.45-7.64 (m, 3H), 7.25-7.45 (m, 2H), 7.00-7.10 (m, 1H), 6.70-6.95 (m, 1H), 4.25-4.45 (m, 2H), 1.40-1.60 (m, 2H), 1.00-1.30 (m, 2H). ¹⁹F NMR (282 MHz, DMSO-d₆) δ (ppm): −136.09.

### Intermediate 67-1: 3-(3-(4-(aminomethyl)phenyl)-5-(pyridin-3-yl)-3H-imidazo[4,5-b]pyridin-2-yl)pyridin-2-amine

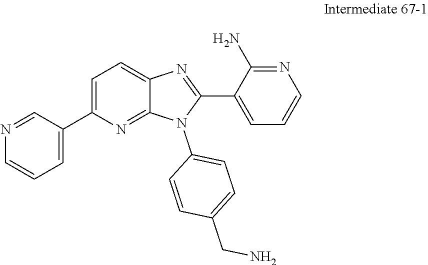 [SMILES: NCc1ccc(-n2c(-c3cccnc3N)nc3ccc(-c4cccnc4)nc32)cc1]

Intermediate 67-1 was prepared in a manner analogous to Intermediate 12-1 using pyridin-3-ylboronic acid in place of (3-morpholinophenyl)boronic acid. MS (ESI) calculated for C₂₃H₁₉N₇: 393.17 m/z, found 394.10 [M+H]⁺.

### Example 68: N-(4-(2-(2-aminopyridin-3-yl)-5-(pyridin-3-yl)-3H-imidazo[4,5-b]pyridin-3-yl)benzyl)-2-(4-formyl-3-hydroxyphenyl)acetamide

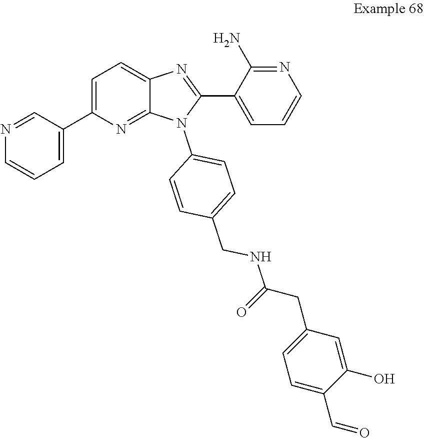 [SMILES: Nc1ncccc1-c1nc2ccc(-c3cccnc3)nc2n1-c1ccc(CNC(=O)Cc2ccc(C=O)c(O)c2)cc1]

Example 68 was prepared in a manner analogous to Example 1 (starting from Step 3) using Intermediate 67-1 in place of the amine starting material. MS (ESI) calculated for C₃₂H₂₅N₇O₃: 555.20 m/z, found 556.20 [M+H]⁺. ¹H NMR (300 MHz, DMSO-d₆) δ (ppm): 10.20 (s, 1H), 9.28 (s, 1H), 8.62-8.80 (m, 1H), 8.45-8.60 (m, 1H), 8.35-8.45 (m, 1H), 8.10-8.20 (m, 1H), 8.00-8.10 (m, 1H), 7.60-7.75 (m, 3H), 7.50-7.60 (m, 2H), 7.40-7.50 (m, 2H), 6.80-7.05 (m, 2H), 6.70-6.80 (m, 1H), 4.35-4.40 (m, 2H), 3.50-3.75 (m, 2H).

### Example 69: N-({4-[2-(2-aminopyridin-3-yl)-5-phenylimidazo[4,5-b]pyridin-3-yl]phenyl}methyl)-1-(4-formyl-3-hydroxyphenyl)cyclopropane-1-carboxamide

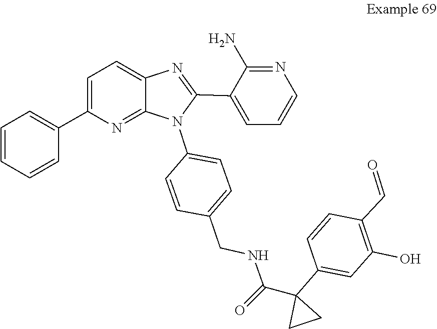 [SMILES: Nc1ncccc1-c1nc2ccc(-c3ccccc3)nc2n1-c1ccc(CNC(=O)C2(c3ccc(C=O)c(O)c3)CC2)cc1]

Example 69 was prepared in a manner analogous to Example 1 (starting from Step 3) using Intermediate 4-1 in place of the amine starting material and Intermediate 69-1 in place of Intermediate 1-4. MS (ESI) calculated for C₃₅H₂₈N₆O₃: 580.22 m/z, found 581.23 [M+H]⁺. ¹H-NMR (400 MHz, DMSO-d₆) δ (ppm): 10.23 (s, 1H), 8.34-8.32 (m, 1H), 8.01-8.07 (m, 4H), 7.65-7.73 (m, 2H), 7.41-7.63 (m, 5H), 7.33-7.40 (m, 2H), 6.97-7.00 (m, 2H), 6.75-6.79 (m, 1H), 4.32 (s, 2H), 1.39-1.42 (m, 2H), 1.04-1.07 (m, 2H).

### Intermediate 69-1: 1-(4-(1,3-dioxolan-2-yl)-3-((4-methoxybenzyl)oxy)phenyl)cyclopropane-1-carboxylic acid

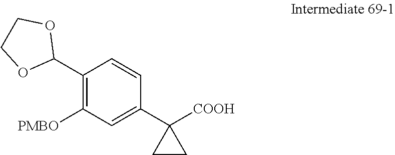

Intermediate 69-1 was prepared in a manner analogous to Intermediate 59-1 using Intermediate 1-3 in place of Intermediate 21-1.MS (ESI) calculated for C₂₁H₂₂O₆: 370.14 m/z, found 371.10 [M+H]⁺.

### Example 70: N-({4-[2-(2-aminopyridin-3-yl)-5-(pyridin-3-yl)imidazo[4,5-b]pyridin-3-yl]phenyl}methyl)-1-(4-formyl-3-hydroxyphenyl)cyclopropane-1-carboxamide

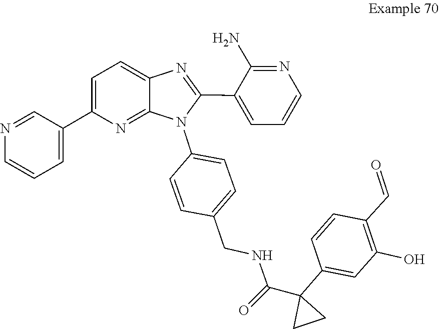 [SMILES: Nc1ncccc1-c1nc2ccc(-c3cccnc3)nc2n1-c1ccc(CNC(=O)C2(c3ccc(C=O)c(O)c3)CC2)cc1]

Example 70 was prepared in a manner analogous to Example 1 (starting from Step 3) using Intermediate 67-1 in place of the amine staring material and Intermediate 69-1 in place of Intermediate 1-4. MS (ESI) calculated for C₃₄H₂₇N₇O₃: 581.22 m/z, found 582.22 [M+H]⁺. ¹H-NMR (400 MHz, DMSO-d₆) δ (ppm): 10.19 (s, 1H), 9.20-9.26 (m, 1H), 8.63-8.70 (m, 2H), 8.40-8.42 (m, 1H), 8.14-8.17 (m, 1H), 8.04-8.06 (m, 1H), 7.74-7.78 (m, 2H), 7.63-7.65 (m, 1H), 7.47-7.49 (m, 2H), 7.32-7.34 (m, 2H), 6.96-6.98 (m, 2H), 6.77-6.81 (m, 1H), 4.31 (s, 2H), 1.38-1.40 (m, 2H) 1.06-1.10 (m, 2H).

### Example 71: N-(4-(2-(2-aminopyridin-3-yl)-5-(pyridin-3-yl)-3H-imidazo[4,5-b]pyridin-3-yl)benzyl)-3-formyl-4-hydroxybenzamide

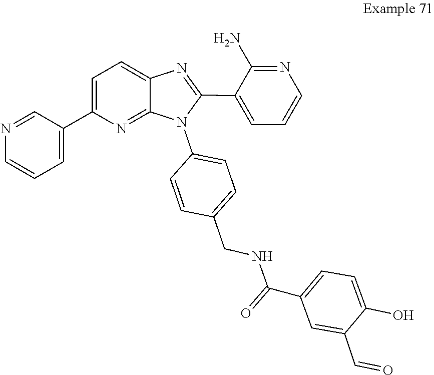 [SMILES: Nc1ncccc1-c1nc2ccc(-c3cccnc3)nc2n1-c1ccc(CNC(=O)c2ccc(O)c(C=O)c2)cc1]

Example 71 was prepared in a manner analogous to Example 12 using Intermediate 67-1 in place of Intermediate 12-1, 3-formyl-4-hydroxybenzoic acid in place of 3-fluoro-5-formyl-4-hydroxybenzoic acid and HOBT in place of HATU. MS (ESI) calculated for C₃H₂₃N₇O₂: 541.19 m/z, found 542.25 [M+H]⁺. ¹H NMR (400 MHz, DMSO-d₆) δ (ppm): 10.32 (s, 1H), 9.25-9.32 (m, 1H), 8.65-8.72 (m, 1H), 8.51-8.65 (m, 1H), 8.38-8.51 (m, 1H), 8.27-8.38 (m, 1H), 8.16-8.27 (m, 1H), 8.01-8.16 (m, 2H), 7.62-7.87 (m, 2H), 7.41-7.62 (m, 4H), 6.97-7.32 (m, 1H), 6.67-6.89 (m, 1H), 4.45-4.78 (m, 2H).

### Example 72: 4-{[(2-{4-[2-(2-aminopyridin-3-yl)-5-phenylimidazo[4,5-b]pyridin-3-yl]phenyl}ethyl)amino]methyl}-2-hydroxybenzaldehyde

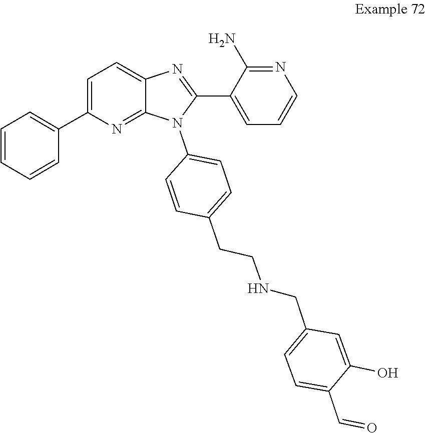 [SMILES: Nc1ncccc1-c1nc2ccc(-c3ccccc3)nc2n1-c1ccc(CCNCc2ccc(C=O)c(O)c2)cc1]

Example 72 was prepared in a manner analogous to Example 19 using Intermediate 36-1 in place of Intermediate 19-2. MS (ESI) calculated for C₃₃H₂₈N₆O₂: 540.23 m/z, found 541.25 [M+H]⁺. ¹H NMR (400 MHz, DMSO-d₆) δ (ppm): 10.30 (s, 1H), 8.30-8.32 (m, 1H), 8.00-8.04 (m, 4H), 7.74-7.76 (m, 1H), 7.11-7.55 (m, 8H), 6.66-7.09 (m, 2H), 6.63-6.64 (m, 1H), 4.21 (s, 2H), 3.27-3.31 (m, 2H), 3.03-3.07 (m, 2H).

### Example 73: N-(4-(2-(2-aminopyridin-3-yl)-5-phenyl-3H-imidazo[4,5-b]pyridin-3-yl)benzyl)-3-formyl-4-hydroxybenzamide

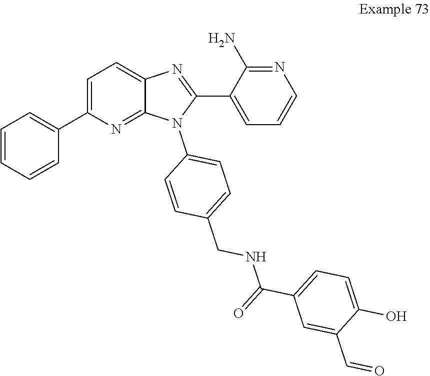 [SMILES: Nc1ncccc1-c1nc2ccc(-c3ccccc3)nc2n1-c1ccc(CNC(=O)c2ccc(O)c(C=O)c2)cc1]

Example 73 was prepared in a manner analogous to Example 12 using Intermediate 4-1 in place of Intermediate 12-1, 3-formyl-4-hydroxybenzoic acid in place of 3-fluoro-5-formyl-4-hydroxybenzoic acid and HOBT in place of HATU. MS (ESI) calculated for C₃₂H₂₄N₆O₃: 540.19 m/z, found 541.20 [M+H]⁺. ¹H NMR (300 MHz, DMSO-d₆) δ (ppm): 10.30 (s, 1H), 8.15-8.43 (m, 2H), 7.90-8.19 (m, 5H), 8.62-8.85 (m, 1H), 7.30-7.60 (m, 7H), 7.05-7.25 (m, 1H), 6.60-6.90 (m, 1H), 4.45-4.80 (m, 2H).

### Example 74: N-({4-[2-(2-aminopyridin-3-yl)imidazo[4,5-b]pyridin-3-yl]phenyl}methyl)-2-fluoro-2-(4-formyl-3-hydroxyphenyl)acetamide

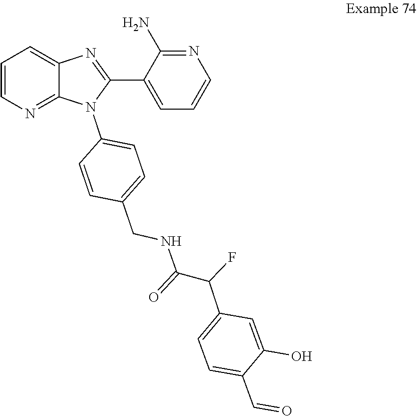 [SMILES: Nc1ncccc1-c1nc2cccnc2n1-c1ccc(CNC(=O)C(F)c2ccc(C=O)c(O)c2)cc1]

Example 74 was prepared in a manner analogous to Example 1 (starting from Step 3) using Intermediate 74-1 in place of the amine starting material and Intermediate 65-1 in place of Intermediate 1-4. MS (ESI) calculated for C₂₇H₂₁FN₆O₃: 496.17 m/z, found 497.15 [M+H]⁺. ¹H-NMR (400 MHz, DMSO-d₆) δ (ppm): 10.16 (s, 1H), 8.37-8.38 (m, 1H), 8.23-8.25 (m, 1H), 7.95-7.97 (m, 1H), 7.70-7.73 (m, 2H), 7.43-7.47 (m, 1H), 7.35-7.36 (m, 4H), 7.04-7.07 (m, 2H), 6.71-6.75 (m, 1H), 5.90-6.01 (m, 1H), 4.39-4.40 (m, 2H). ¹⁹F NMR (300 MHz, DMSO-d₆) δ (ppm): 181.28.

### Intermediate 74-1: 3-(3-(4-(aminomethyl)phenyl)-3H-imidazo[4,5-b]pyridin-2-yl)pyridin-2-amine

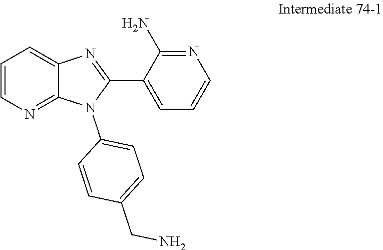 [SMILES: NCc1ccc(-n2c(-c3cccnc3N)nc3cccnc32)cc1]

### Synthetic Route:

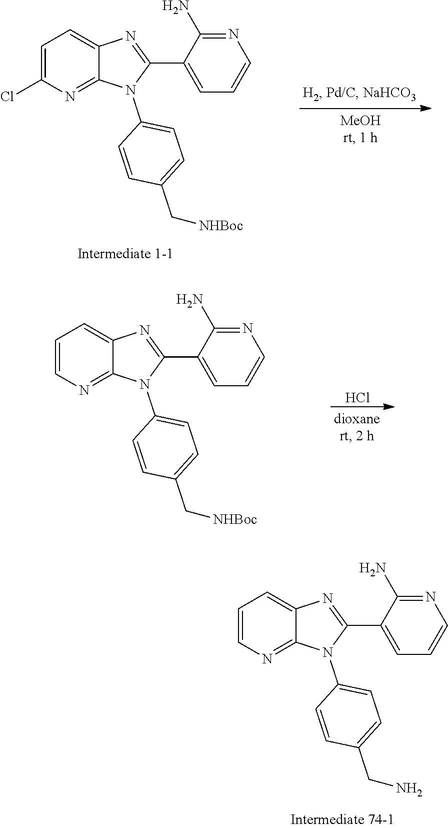 [SMILES: CC(C)(C)OC(=O)NCc1ccc(-n2c(-c3cccnc3N)nc3ccc(Cl)nc32)cc1 ; CC(C)(C)OC(=O)NCc1ccc(-n2c(-c3cccnc3N)nc3cccnc32)cc1 ; Cl ; NCc1ccc(-n2c(-c3cccnc3N)nc3cccnc32)cc1]

### Step 1: Synthesis of tert-butyl (4-(2-(2-aminopyridin-3-yl)-3H-imidazo[4,5-b]pyridin-3-yl) benzyl)carbamate

To a solution of tert-butyl (4-(2-(2-aminopyridin-3-yl)-5-chloro-3H-imidazo[4,5-b]pyridin-3-yl)benzyl)carbamate (Intermediate 1-1) (100 mg, 0.285 mmol, 1 equiv) in methanol (10 mL) was added palladium on carbon (10%, 50 mg, 0.15 equiv) and sodium bicarbonate (96 mg, 1.1 mmol, 4 equiv) under nitrogen atmosphere. The mixture stirred under hydrogen atmosphere at room temperature for 1 h. The resulting mixture was filtered through celite and concentrated in vacuo to afford 3-{3-[4-(aminomethyl)phenyl]imidazo[4,5-b]pyridin-2-yl}pyridin-2-amine (80 mg, 89%) as a yellow solid, which was used in the next step without further purification. MS (ESI) calculated for C₁₈H₁₆N₆: 316.16 m/z, found 317.15 [M+H]⁺.

### Step 2: Synthesis of 3-(3-(4-(aminomethyl)phenyl)-3H-imidazo[4,5-b]pyridin-2-yl)pyridin-2-amine (Intermediate 74-1)

A solution of tert-butyl N-({4-[2-(2-aminopyridin-3-yl)imidazo[4,5-b]pyridin-3-yl]phenyl}methyl)carbamate (300 mg, 0.720 mmol, 1 equiv) in 4N hydrochloric acid in 1,4-dioxane (5 mL) was stirred at room temperature for 2 h. The resulting mixture was concentrated in vacuo and purified by reverse-phase flash column chromatography on C18 silica gel using a 20-95% gradient of acetonitrile in water (+0.05% ammonium bicarbonate) with a 10 minute hold at 70% acetonitrile to afford 3-{3-[4-(aminomethyl)phenyl]imidazo[4,5-b]pyridin-2-yl}pyridin-2-amine (Intermediate 74-1) as a yellow solid (200 mg, 88%). MS (ESI) calculated for C₁₈H₁₆N₆: 316.14 m/z, found 317.05 [M+H]⁺.

### Example 75: N-(4-(2-(2-aminopyridin-3-yl)-5-phenyl-3H-imidazo[4,5-b]pyridin-3-yl) benzyl)-2-(5-formyl-6-hydroxypyridin-2-yl)acetamide

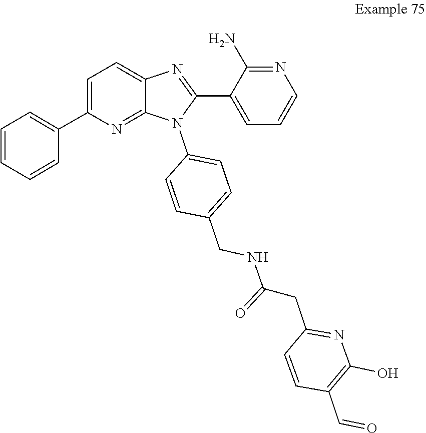 [SMILES: Nc1ncccc1-c1nc2ccc(-c3ccccc3)nc2n1-c1ccc(CNC(=O)Cc2ccc(C=O)c(O)n2)cc1]

Example 75 was prepared in a manner analogous to Example 1 (starting from Step 3) using Intermediate 4-1 in place of the amine starting material, Intermediate 75-1 in place of Intermediate 1-4 and hydrogen bromide (33% in acetic acid) overnight instead of 2,2,2-trifluoroacetic acid/methanesulfonic acid for 1 h. MS (ESI) calculated for C₃₂H₂₅N₇O₃: 555.20 m/z, found 556.15 [M+H]⁺. ¹H NMR (300 MHz, DMSO-d₆) δ (ppm): 10.09 (s, 1H), 8.20-8.40 (m, 1H), 7.85-8.05 (m, 5H), 7.55-7.75 (m, 1H), 7.35-7.52 (m, 7H), 6.70-6.80 (m, 1H), 6.30-6.40 (m, 1H), 4.41 (s, 2H), 3.57 (s, 2H).

### Intermediate 75-1: 2-(5-(1,3-dioxolan-2-yl)-6-methoxypyridin-2-yl)acetic acid

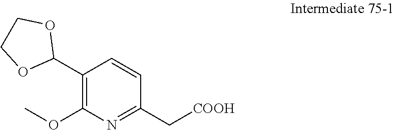 [SMILES: COc1nc(CC(=O)O)ccc1C1OCCO1]

### Synthetic Route:

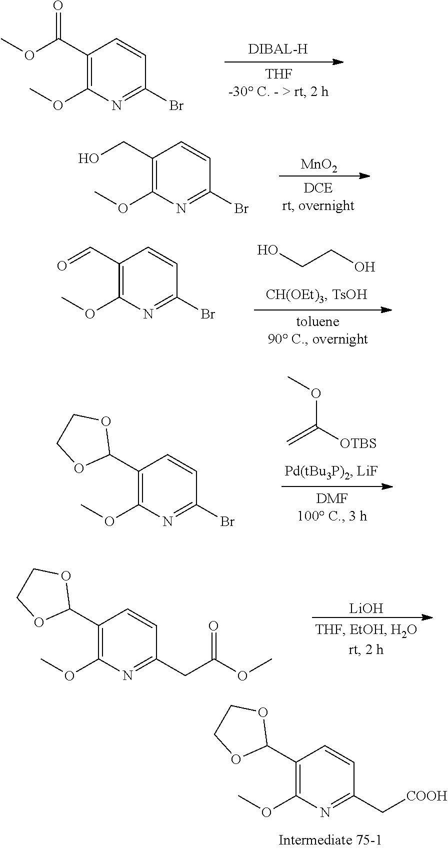

### Step 1: Synthesis of (6-bromo-2-methoxypyridin-3-yl)methanol

To a cooled (−30° C.) mixture of methyl 6-bromo-2-methoxypyridine-3-carboxylate (4.00 g, 16.3 mmol, 1 equiv) in tetrahydrofuran (94 mL) was added di-iso-butyl aluminum hydride (1M in toluene, 49 mL, 49 mmol, 3 equiv) dropwise with stirring. The resulting mixture was stirred for an additional 2 hours at room temperature and the reaction was quenched by the addition of saturated aqueous potassium sodium tartrate (200 mL) and methanol (100 mL) at 0° C. The resulting mixture was extracted with ethyl acetate (3×100 mL). The combined organic layers were washed with brine (100 mL), dried over anhydrous sodium sulfate, filtered, and concentrated under reduced pressure to afford (6-bromo-2-methoxypyridin-3-yl) methanol (3.93 g, crude quant.) as a pale-yellow solid, which was used without further purification in the next step. MS (ESI) calculated for C₇H₈BrNO₂: 216.97 m/z, found 218.05, 220.05 [M+H, M+2+H]⁺.

### Step 2: Synthesis of 6-bromo-2-methoxypyridine-3-carbaldehyde

A suspension of (6-bromo-2-methoxypyridin-3-yl) methanol (54 mg, 0.25 mmol, 1 equiv) and manganese (IV) oxide (322 mg, 3.71 mmol, 15 equiv) in 1,2-dichloroethane (2 mL) was stirred overnight at room temperature. The resulting mixture was filtered, rinsing with ethyl acetate (3×50 mL). The filtrate was concentrated under reduced pressure and the residue was purified by silica gel column chromatography using a 0-20% gradient of ethyl acetate in petroleum ether to afford 6-bromo-2-methoxypyridine-3-carbaldehyde (2.84 g, 74%) as a pale-yellow solid. MS (ESI) calculated for C₇H₆BrNO₂: 214.96 m/z, found 216.05, 218.10 [M+H, M+2+H]⁺.

### Step 3: Synthesis of 6-bromo-3-(1,3-dioxolan-2-yl)-2-methoxypyridine

A solution of 6-bromo-2-methoxypyridine-3-carbaldehyde (2.84 g, 13.1 mmol, 1 equiv) in toluene (150 mL) was treated with ethylene glycol (2.45 g, 39.4 mmol, 3 equiv) and p-toluenesulfonic acid (234 mg, 1.36 mmol, 0.10 equiv). The resulting mixture was stirred for 10 min at room temperature then overnight at 90° C. The mixture was allowed to cool to room temperature and was quenched with water (200 mL). The resulting mixture was extracted with ethyl acetate (3×100 mL). The combined organic layers were washed with brine (100 mL), dried over anhydrous sodium sulfate, filtered, and concentrated in vacuo. The residue was purified by silica gel column chromatography, using a 0-15% gradient of ethyl acetate in petroleum ether to afford 6-bromo-3-(1,3-dioxolan-2-yl)-2-methoxypyridine (3.2 g, 94%) as a pale-yellow oil. MS (ESI) calculated for C₉H₁₀BrNO₃: 258.98 m/z, found 259.90, 261.90 [M+H, M+2+H]⁺.

### Step 4: Synthesis of methyl 2-(5-(1,3-dioxolan-2-yl)-6-methoxypyridin-2-yl)acetate

A solution of 6-bromo-3-(1,3-dioxolan-2-yl)-2-methoxypyridine (815 mg, 3.13 mmol, 1 equiv), bis(tri-tert-butylphosphine)palladium(0) (320 mg, 0.627 mmol, 0.2 equiv), lithium fluoride (325 mg, 12.5 mmol, 4 equiv) and tert-butyl[(1-methoxyethenyl)oxy]dimethylsilane (4.721 mg, 25.07 mmol, 8 equiv) in N,N-dimethylformamide (8 mL) was stirred for 3 hours at 100° C. under nitrogen atmosphere. The reaction was quenched with water at room temperature. The resulting mixture was extracted with dichloromethane (3×50 mL). The combined organic layers were washed with brine (50 mL), dried over anhydrous sodium sulfate, filtered, and concentrated under reduced pressure. The residue was purified by silica gel column chromatography using a 0-45% gradient of 10:1 dichloromethane/methanol in petroleum ether to afford methyl 2-[5-(1,3-dioxolan-2-yl)-6-methoxypyridin-2-yl]acetate (900 mg, quant.) as a reddish-brown oil. MS (ESI) calculated for C₁₂H₁₅NO₅: 253.10 m/z, found 254.15 [M+H]⁺.

### Step 5: Synthesis of [5-(1,3-dioxolan-2-yl)-6-methoxypyridin-2-yl]acetic acid (Intermediate 75-1)

To a cooled (0° C.) solution of methyl 2-[5-(1,3-dioxolan-2-yl)-6-methoxypyridin-2-yl]acetate (1.29 g, 5.09 mmol, 1 equiv) in tetrahydrofuran (10 mL) and ethanol (10 mL) was added a solution of lithium hydroxide (0.24 g, 10 mmol, 2 equiv) in water (5 mL) dropwise. The resulting mixture was stirred for 1 h at room temperature. The mixture was partially concentrated and brought to a pH of 6 with 0.5 M hydrochloric acid. The resulting mixture was concentrated under reduced pressure and suspended in dichloromethane/methanol (20 mL). The mixture was filtered, rinsing with dichloromethane/methanol (3×20 mL). The filtrate was concentrated under reduced pressure to afford [5-(1,3-dioxolan-2-yl)-6-methoxypyridin-2-yl]acetic acid (Intermediate 75-1) (550 mg, 38%) as brown/yellow solid, which was used in subsequent transformations without further purification. MS (ESI) calculated for C₁₁H₁₃NO₅: 239.08 m/z, found 240.05 [M+H]⁺.

### Example 76: 4-{2-[({4-[2-(2-aminopyridin-3-yl)-5-phenylimidazo[4,5-b]pyridin-3-yl]phenyl}methyl)amino]ethyl}-2-hydroxy-3-methoxybenzaldehyde

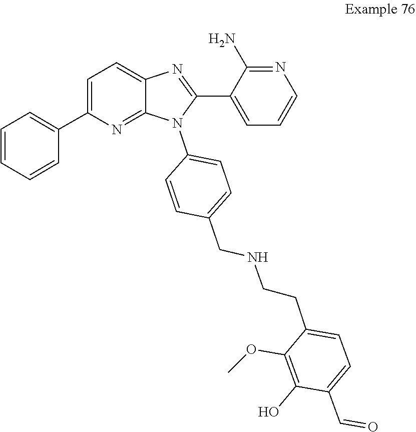 [SMILES: COc1c(CCNCc2ccc(-n3c(-c4cccnc4N)nc4ccc(-c5ccccc5)nc43)cc2)ccc(C=O)c1O]

Example 76 was prepared in a manner analogous to Example 19 using Intermediate 4-1 in place of Intermediate 19-2, Intermediate 76-1 in place of Intermediate 19-3 and dichloromethane/2,2,2-trifluoroacetic acid (15:1) in place of 2,2,2-trifluoroacetic acid/methanesulfonic acid. MS (ESI) calculated for C₃₄H₃₀N₆O₃: 570.24 m/z, found 571.25 [M+H]⁺. ¹H NMR (300 MHz, DMSO-d₆) δ ppm: 10.07 (m, 1H), 8.23-8.33 (m, 1H), 7.85-8.11 (m, 4H), 7.39-7.52 (m, 8H), 7.09-7.21 (m, 1H), 6.86-7.06 (m, 1H), 6.23-6.38 (m, 1H), 3.69-3.85 (m, 5H), 2.76-2.88 (m, 4H).

### Intermediate 76-1: 2-(3-((tert-butyldimethylsilyl)oxy)-4-(1,3-dioxolan-2-yl)-2-methoxy phenyl)acetaldehyde

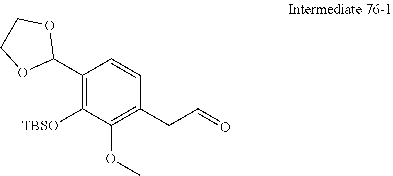 [SMILES: COc1c(CC=O)ccc(C2OCCO2)c1O[Si](C)(C)C(C)(C)C]

### Synthetic Route:

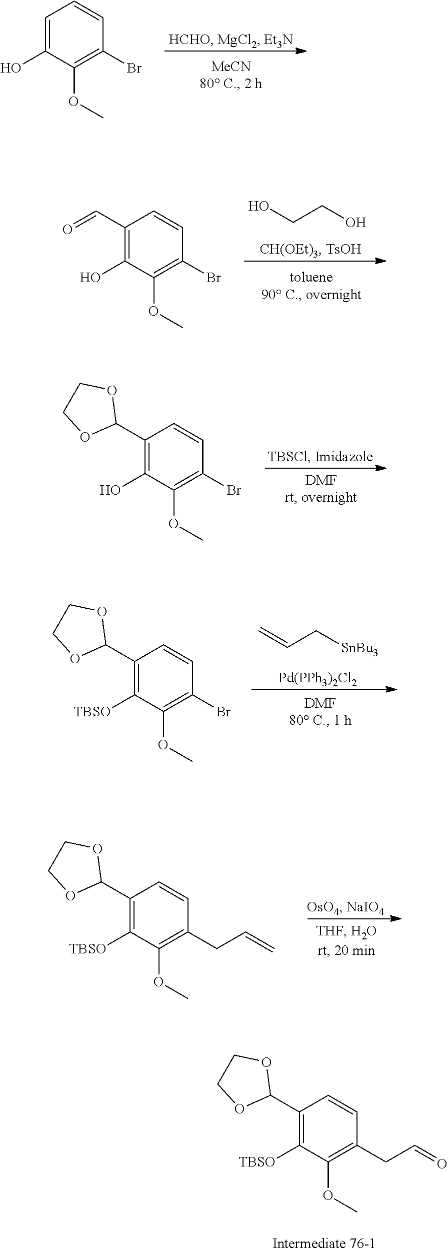

### Step 1: Synthesis of 4-bromo-2-hydroxy-3-methoxybenzaldehyde

To a solution of 3-bromo-2-methoxyphenol (2.00 g, 9.85 mmol, 1 equiv) in acetonitrile (15 mL) were added paraformaldehyde (4.44 g, 49.3 mmol, 5 equiv), magnesium(II) chloride (1.41 g, 14.8 mmol, 1.5 equiv) and triethylamine (2.49 g, 24.6 mmol, 2.5 equiv). The obtained suspension was stirred at room temperature for 10 min then at 80° C. for 2 h. The resulting mixture was cooled to 0° C. and quenched by the addition of hydrochloric acid (2 M). The resulting mixture was extracted with dichloromethane (30 mL×3). The combined organic layers were washed with water (20 mL×3), dried over anhydrous sodium sulfate, filtered, and concentrated under reduced pressure. The residue was purified by silica gel column chromatography using a 0-7% gradient of ethyl acetate in petroleum ether to provide 4-bromo-2-hydroxy-3-methoxybenzaldehyde (1.28 g, 56%) as a light-yellow oil. MS (ESI) calculated for C₈H₇BrO₃: 229.96 m/z, found 230.90 [M+H]⁺.

### Step 2: Synthesis of 3-bromo-6-(1,3-dioxolan-2-yl)-2-methoxyphenol

To a colorless solution of 4-bromo-2-hydroxy-3-methoxybenzaldehyde (1.28 g, 5.54 mmol, 1 equiv) in toluene (13 mL) were added ethylene glycol (1.72 g, 27.7 mmol, 5 equiv), triethyl orthoformate (2.46 g, 16.6 mmol, 3 equiv) and p-toluenesulfonic acid (0.10 g, 0.55 mmol, 0.1 equiv). The obtained solution was stirred at room temperature for 10 min then overnight at 90° C. The resulting solution was cooled to 0° C. and quenched by the addition of water (5 mL). The resulting mixture was extracted with ethyl acetate (10 mL×3). The combined organic layers were washed with brine (10 mL×3), dried over anhydrous sodium sulfate, filtered, and concentrated under reduced pressure. The residue was purified by silica gel column chromatography using a 0-7% gradient of ethyl acetate in petroleum ether to provide 3-bromo-6-(1,3-dioxolan-2-yl)-2-methoxyphenol (0.58 g, 38%) as a white solid. MS (ESI) calculated for C₁₀H₁₁BrO₄: 273.98 m/z, found 274.90 [M+H]⁺.

### Step 3: Synthesis of 3-bromo-6-(1,3-dioxolan-2-yl)-2-methoxyphenoxy(tert-butyl)dimethylsilane

To a solution of 3-bromo-6-(1,3-dioxolan-2-yl)-2-methoxyphenol (0.58 g, 2.11 mmol, 1 equiv) and imidazole (0.29 g, 4.2 mmol, 2 equiv) in N,N-dimethylformamide (15 mL) was added tert-butyldimethylsilyl chloride (0.44 g, 3.0 mmol, 1.4 equiv). The resulting mixture was stirred overnight at room temperature then cooled to 0° C. and quenched by the addition of water (20 mL). The resulting mixture was extracted with ethyl acetate (30 mL×3). The combined organic layers were washed with brine (30 mL×3), dried over anhydrous sodium sulfate, filtered, and concentrated under reduced pressure. The residue was purified by silica gel column chromatography using a 0-7% gradient of ethyl acetate in petroleum ether to provide 3-bromo-6-(1,3-dioxolan-2-yl)-2-methoxyphenoxy(tert-butyl)dimethylsilane (0.57 g, 69%) as a white solid. MS (ESI) calculated for C₁H₂₅BrO₄Si: 388.07 m/z, found 389.10 [M+H]⁺.

### Step 4: Synthesis of tert-butyl(6-(1,3-dioxolan-2-yl)-2-methoxy-3-(prop-2-en-1-yl)phenoxy)dimethylsilane

To a solution of 3-bromo-6-(1,3-dioxolan-2-yl)-2-methoxyphenoxy(tert-butyl)dimethylsilane (0.64 g, 1.6 mmol, 1 equiv) and tributyl(prop-2-en-1-yl)stannane (1.09 g, 3.29 mmol, 2 equiv) in N,N-dimethylformamide (6 mL) was added bis(triphenylphosphine )palladium(II) dichloride (0.12 g, 0.16 mmol, 0.1 equiv). After stirring for 1 h at 80° C. under a nitrogen atmosphere, the mixture was cooled to 0° C. and quenched by the addition of saturated aqueous ammonium chloride (10 mL). The resulting mixture was extracted with ethyl acetate (20 mL×3). The combined organic layers were washed with water (10 mL×3), dried over anhydrous sodium sulfate, filtered, and concentrated under reduced pressure. The residue was purified by silica gel column chromatography using a 0-30% gradient of ethyl acetate in petroleum ether to provide tert-butyl(6-(1,3-dioxolan-2-yl)-2-methoxy-3-(prop-2-en-1-yl)phenoxy)dimethylsilane (0.57 g, 99%) as a white solid. MS (ESI) calculated for C₁₉H₃₀O₄Si: 350.19 m/z, found 351.20 [M+H]⁺.

### Step 5: Synthesis of 2-{3-[(tert-butyldimethylsilyl)oxy]-4-(1,3-dioxolan-2-yl)-2-methoxyphenyl}acetaldehyde (Intermediate 76-1)

To a solution of tert-butyl(6-(1,3-dioxolan-2-yl)-2-methoxy-3-(prop-2-en-1-yl) phenoxy)dimethylsilane (0.54 g, 1.5 mmol, 1 equiv) in tetrahydrofuran (10 mL) and water (10 mL) were added osmium tetroxide (0.54 mL, 10 mmol, 6.8 equiv) and sodium periodate (0.99 g, 4.6 mmol, 3 equiv). After stirring for 30 min at room temperature, the reaction was quenched by addition of water. The resulting mixture was extracted with ethyl acetate (60 mL×3). The combined organic layers were washed with water (50 mL×3), dried over anhydrous sodium sulfate, filtered, and concentrated under reduced pressure. The residue was purified by silica gel column chromatography using a 0-25% gradient of ethyl acetate in petroleum ether to provide 2-{3-[(tert-butyldimethylsilyl)oxy]-4-(1,3-dioxolan-2-yl)-2-methoxyphenyl}acetaldehyde (Intermediate 76-1) (0.12 g, 22%) as a light-yellow solid. MS (ESI) calculated for C₁₈H₂₈O₅Si: 352.17 m/z, found 353.20 [M+H]⁺.

### Example 77: N-(4-(2-(2-aminopyridin-3-yl)-5-phenyl-3H-imidazo[4,5-b]pyridin-3-yl)benzyl)-2-(6-formyl-5-hydroxypyridin-3-yl)acetamide

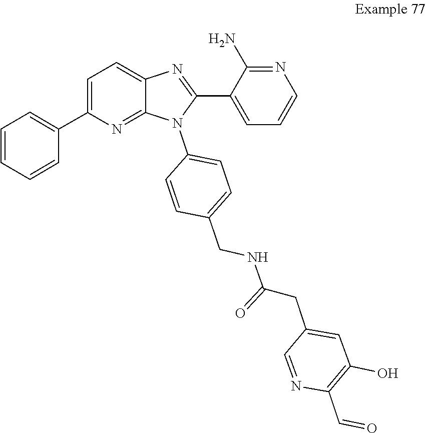 [SMILES: Nc1ncccc1-c1nc2ccc(-c3ccccc3)nc2n1-c1ccc(CNC(=O)Cc2cnc(C=O)c(O)c2)cc1]

Example 77 was prepared in a manner analogous to Example 1 (starting from Step 3) using Intermediate 4-1 in place of the amine starting material and Intermediate 77-1 in place of Intermediate 1-4. Mass (ESI) calculated for C₃₂H₂₅N₇O₃: 555.20, found 556.10 [M+H]⁺. ¹H-NMR (400 MHz, DMSO-d₆) δ (ppm): 10.04 (s, 1H), 8.25-8.27 (m, 1H), 7.97-8.02 (m, 5H), 7.34-7.43 (m, 8H), 7.20-7.22 (m, 1H), 6.30-6.42 (m, 1H), 4.39-4.40 (s, 2H), 3.50 (s, 2H).

### Intermediate 77-1: 2-(5-(benzyloxy)-6-(1,3-dioxolan-2-yl)pyridin-3-yl)acetic acid

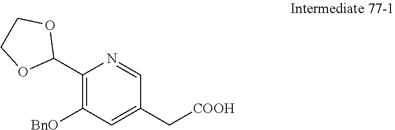 [SMILES: O=C(O)Cc1cnc(C2OCCO2)c(OCc2ccccc2)c1]

Intermediate 77-1 was prepared in a manner analogous to Intermediate 56-1 using benzyl bromide in place of p-methoxybenzyl chloride/potassium iodide and omitting Step 6 (cyclopropanation). MS (ESI) calculated for C₁₇H₁₇NO₅: 315.11 m/z, found 316.00 [M+H]⁺.

### Example 78: N-(4-(2-(2-aminopyridin-3-yl)-3H-imidazo[4,5-b]pyridin-3-yl)benzyl)-2,2-difluoro-2-(4-formyl-3-hydroxyphenyl)acetamide

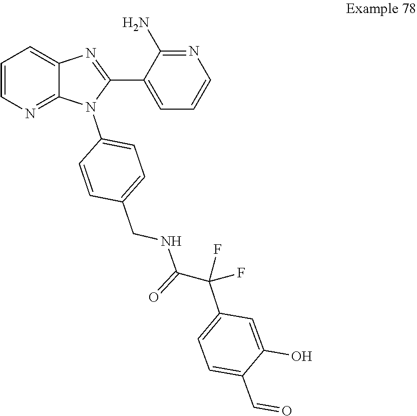 [SMILES: Nc1ncccc1-c1nc2cccnc2n1-c1ccc(CNC(=O)C(F)(F)c2ccc(C=O)c(O)c2)cc1]

Example 78 was prepared in a manner analogous to Example 1 (starting from Step 3) using Intermediate 74-1 in place of the amine starting material and Intermediate 78-1 in place of Intermediate 1-4. MS (ESI) calculated for C₂₇H₂₀F₂N₆O₃: 514.16 m/z, found 515.10 [M+H]⁺. ¹H NMR (300 MHz, DMSO-d₆) δ (ppm): 10.35 (s, 1H), 9.68-9.78 (m, 1H), 8.16-8.50 (m, 2H), 7.90-8.10 (m, 1H), 7.70-7.75 (m, 1H), 7.33-7.50 (m, 5H), 7.20-7.28 (m, 2H), 7.15-7.20 (m, 1H), 6.95-6.90 (m, 2H), 6.45-6.35 (m, 1H), 4.45-4.38 (m, 2H). ¹⁹F NMR (300 MHz, DMSO-d₆) δ (ppm): −102.77.

### Intermediate 78-1: 2-(4-(1,3-dioxolan-2-yl)-3-((4-methoxybenzyl)oxy)phenyl)-2,2-difluoroacetic acid

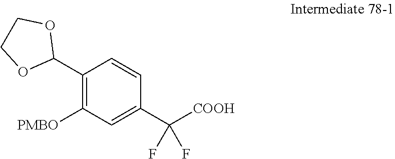

### Synthetic Route:

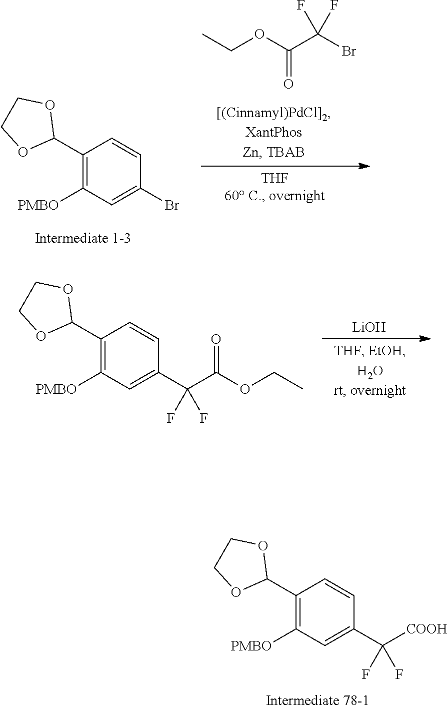

### Step 1: Synthesis of ethyl 2-(4-(1,3-dioxolan-2-yl)-3-((4-methoxybenzyl)oxy)phenyl)-2,2-difluoroacetate

A mixture of 2-{4-bromo-2-[(4-methoxyphenyl)methoxy]phenyl}-1,3-dioxolane (Intermediate 1-3) (500 mg, 1.37 mmol, 1 equiv), ethyl 2-bromo-2,2-difluoroacetate (834 mg, 4.11 mmol, 3 equiv), XantPhos (119 mg, 0.205 mmol, 0.15 equiv), zinc (269 mg, 4.11 mmol, 3 equiv), tetra-n-butylammonium bromide (662 mg, 2.05 mmol, 1.5 equiv) and palladium(71-cinnamyl) chloride dimer (35 mg, 0.068 mmol, 0.05 equiv) in tetrahydrofuran (20 mL) was stirred overnight at 60° C. under nitrogen atmosphere. The mixture was cooled to room temperature and quenched with water (20 mL). The resulting mixture was extracted with ethyl acetate (20 mL×3). The combined organic layers were dried over anhydrous sodium sulfate, filtered, and concentrated under reduced pressure. The residue was purified by silica gel column chromatography using a 0-100% gradient of ethyl acetate in petroleum ether to afford ethyl 2-[4-(1,3-dioxolan-2-yl)-3-[(4-methoxyphenyl)methoxy]phenyl]-2,2-difluoroacetate (100 mg, 18%) as a colorless oil. MS (ESI) calculated for C₂₁H₂₂F₂O₆: 408.14 m/z, found 409.20 [M+H]⁺.

### Step 2: Synthesis of 2-(4-(1,3-dioxolan-2-yl)-3-((4-methoxybenzyl)oxy)phenyl)-2,2-difluoroacetic acid (Intermediate 78-1)

To a solution of ethyl 2-[4-(1,3-dioxolan-2-yl)-3-[(4-methoxyphenyl)methoxy]phenyl]-2,2-difluoroacetate (180 mg 44.1 mmol, 1 equiv) in tetrahydrofuran (10 mL) and ethanol (0.9 mL) was added a solution of lithium hydroxide (88 mg, 88 mmol, 2 equiv) in water (2.5 mL) and the resulting mixture was stirred overnight at room temperature. The reaction was quenched with water (30 mL) and adjusted to pH 6-7 with 1M hydrochloric acid. The mixture was extracted with ethyl acetate (3×40 mL). The combined organic extracts were washed with brine (20 mL), dried over anhydrous sodium sulfate, filtered, and concentrated under reduced pressure to afford 2-(4-(1,3-dioxolan-2-yl)-3-((4-methoxybenzyl)oxy)phenyl)-2,2-difluoroacetic acid (Intermediate 78-1) (80 mg, 48%) as a colorless oil, which was used without further purification in subsequent transformations. MS (ESI) calculated for C₁₉H₁₈F₂O₆: 380.11 m/z, found 379.10 [M−H]⁻.

### Example 79: N-({4-[2-(2-aminopyridin-3-yl)-5-(pyridin-3-yl)imidazo[4,5-b]pyridin-3-yl]phenyl}methyl)-2-(2-fluoro-4-formyl-3-hydroxyphenyl)acetamide

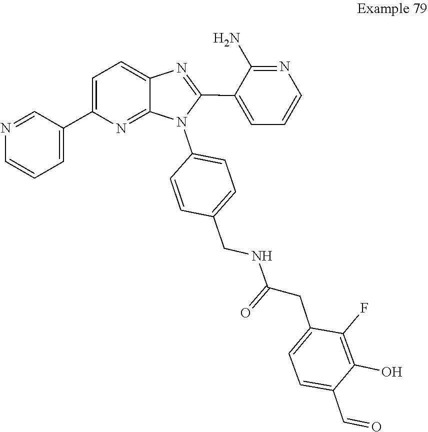 [SMILES: Nc1ncccc1-c1nc2ccc(-c3cccnc3)nc2n1-c1ccc(CNC(=O)Cc2ccc(C=O)c(O)c2F)cc1]

Example 79 was prepared in a manner analogous to Example 1 (starting from Step 3) using Intermediate 67-1 in place of the amine starting material and Intermediate 21-2 in place of Intermediate 1-4. MS (ESI) calculated for C₃₂H₂₄FN₇03: 573.19 m/z, found 574.20 [M+H]⁺. ¹H NMR (400 MHz, DMSO-d₆) δ (ppm): 10.20 (s, 1H), 9.20-9.22 (m, 1H), 8.58-8.60 (m, 1H), 8.30-8.38 (m, 2H), 8.00-8.08 (m, 2H), 7.40-7.52 (m, 6H), 7.22-7.25 (m, 1H), 6.87-6.92 (m, 1H), 6.41-6.45 (m, 1H), 4.41-4.42 (m, 2H), 3.59-3.65 (m, 2H). ¹⁹F-NMR (400 MHz, DMSO-d₆) δ (ppm): −139.31.

### Example 80: 4-{2-[({4-[2-(2-aminopyridin-3-yl)imidazo[4,5-b]pyridin-3-yl]phenyl}methyl) amino]ethyl}-2-(difluoromethyl)benzaldehyde

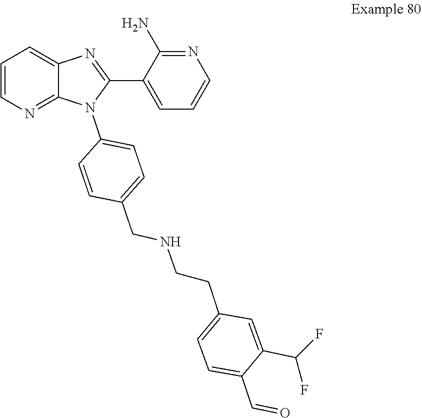 [SMILES: Nc1ncccc1-c1nc2cccnc2n1-c1ccc(CNCCc2ccc(C=O)c(C(F)F)c2)cc1]

Example 80 was prepared in a manner analogous to Example 19 using Intermediate 74-1 in place of Intermediate 19-2, Intermediate 80-2 in place of Intermediate 19-3 and dichloromethane/2,2,2-trifluoroacetic acid (10:1) in place of 2,2,2-trifluoroacetic acid/methanesulfonic acid. MS (ESI) calculated for C₂₈H₂₄F₂N₆O: 498.20 m/z, found 499.20 [M+H]⁺. ¹H NMR (300 MHz, DMSO-d₆) δ (ppm): 10.14 (s, 1H), 8.31-8.33 (m, 1H), 8.18-8.21 (m, 1H), 7.96-8.00 (m, 2H), 7.58-7.76 (m, 3H), 7.38-7.46 (m, 2H), 7.35-7.36 (m, 1H), 7.28-7.32 (m, 2H), 6.97-7.19 (m, 1H), 6.32-6.36 (m, 1H), 3.79 (s, 2H), 2.92-2.94 (m, 2H), 2.78-2.89 (m, 2H).

### Intermediate 80-2: 2-(3-(difluoromethyl)-4-(1,3-dioxolan-2-yl)phenyl)acetaldehyde

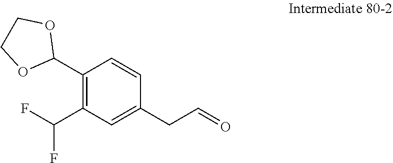 [SMILES: O=CCc1ccc(C2OCCO2)c(C(F)F)c1]

Intermediate 80-2 was prepared in a manner analogous to Intermediate 16-4 using Intermediate 80-1 in place of Intermediate 16-3. MS (ESI) calculated for C₁₂H₁₂F₂O₃: 242.08 m/z, found 243.10 [M+H]*.

### Intermediate 80-1: 2-(4-bromo-2-(difluoromethyl)phenyl)-1,3-dioxolane

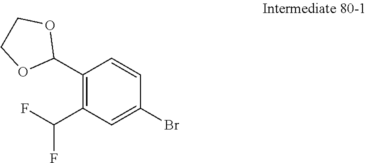 [SMILES: FC(F)c1cc(Br)ccc1C1OCCO1]

### Synthetic Route:

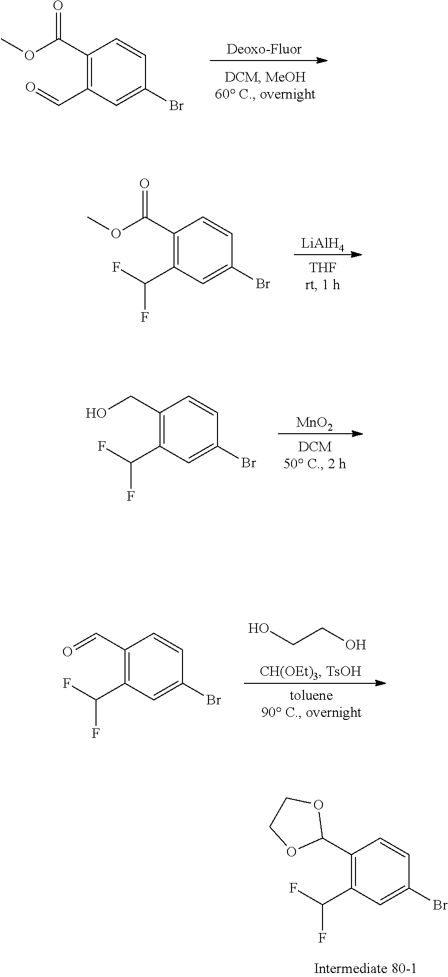

### Step 1: Synthesis of methyl 4-bromo-2-(difluoromethyl)benzoate

To a solution of methyl 4-bromo-2-formylbenzoate (1.80 g, 7.41 mmol, 1 equiv) in dichloromethane were added Deoxo-Fluor (4.92 g, 22.2 mmol, 3 equiv) and methanol (0.06 mL) and the resulting mixture was stirred at 60° C. overnight. The mixture was cooled room temperature and brough to pH ˜8 with saturated aqueous sodium bicarbonate. The resulting mixture was extracted with dichloromethane (100 mL×3). The combined organic layers were washed with water (100 mL×3), dried over anhydrous sodium sulfate, filtered, and concentrated under reduced pressure. The residue was purified by silica gel column chromatography using a 0-16% gradient of ethyl acetate in petroleum ether to afford methyl 4-bromo-2-(difluoromethyl)benzoate (1.6 g, 82%) as a white solid. ¹H NMR (300 MHz, DMSO-d₆) δ (ppm): 7.92-7.95 (m, 3H), 7.33-7.69 (m, 1H), 3.87 (s, 3H).

### Step 2: Synthesis of [4-bromo-2-(difluoromethyl)phenyl]methanol

To a cooled (0° C.) solution of methyl 4-bromo-2-(difluoromethyl)benzoate (1.5 g, 5.7 mmol, 1 equiv) in tetrahydrofuran (100 mL) was added lithium aluminum hydride (1M in tetrahydrofuran, 2.5 mL, 2.5 mmol, 0.5 equiv) under nitrogen atmosphere and the obtained solution was stirred at room temperature for 2 h. The reaction was quenched by the addition of water. The resulting mixture was extracted with ethyl acetate (150 mL×3). The combined organic layers were washed with water (150 mL×3), dried over anhydrous sodium sulfate, filtered, and concentrated under reduced pressure. The residue was purified by silica gel column chromatography using a 0-30% gradient of ethyl acetate in petroleum ether to provide [4-bromo-2-(difluoromethyl)phenyl]methanol (1.3 g, 97%) as a colorless oil. MS (ESI) calculated for C₈H₇BrF₂O: 235.96 m/z, found 234.90 [M−H]⁻.

### Step 3: Synthesis of 4-bromo-2-(difluoromethyl)benzaldehyde

To a solution of [4-bromo-2-(difluoromethyl)phenyl]methanol (1.3 g, 5.5 mmol, 1 equiv) in 1,2-dichloroethane (50 mL) was added manganese (IV) oxide (10.0 g, 115 mmol, 21 equiv). After stirring for 2 h at 50° C., the mixture was concentrated under reduced pressure. Water was added and the resulting mixture was extracted with ethyl acetate (100 mL×3). The combined organic layers were washed with brine, dried over sodium sulfate, filtered, and concentrated under reduced pressure. The residue was purified by silica gel column chromatography using a 0-30% gradient of ethyl acetate in petroleum ether to provide 4-bromo-2-(difluoromethyl)benzaldehyde (1.0 g, 78%) as a light-yellow solid. MS (ESI) calculated for C₈H₅BrF₂O: 233.95 m/z, found 232 90 [M−H]⁻.

### Step 4: Synthesis of 2-[4-bromo-2-(difluoromethyl)phenyl]-1,3-dioxolane (Intermediate 80-1)

To a solution of 4-bromo-2-(difluoromethyl)benzaldehyde (60 mg, 0.26 mmol, 1 equiv) in toluene (4 mL) were added ethylene glycol (79 mg, 1.3 mmol, 5 equiv), triethyl orthoformate (113 mg, 0.765 mmol, 3 equiv) and p-toluenesulfonic acid (4.4 mg, 0.026 mmol, 0.1 equiv) and the resulting was stirred at room temperature for 10 min then at 90° C. overnight. The resulting solution was cooled to 0° C. and quenched by the addition of water (5 mL). The mixture was extracted with ethyl acetate (10 mL×3). The combined organic layers were washed with water (10 mL×3), dried over anhydrous sodium sulfate, filtered, and concentrated under reduced pressure. The residue was purified by silica gel column chromatography using a 0-7% gradient of ethyl acetate in petroleum ether to provide 2-[4-bromo-2-(difluoromethyl)phenyl]-1,3-dioxolane (Intermediate 80-1) (40 mg, 56%)) as a white solid. MS (ESI) calculated for C₁₀H₉BrF₂O₂: 277.98 m/z, found 278.90 [M+H]⁺.

### Example 81: 5-(2-((4-(2-(2-aminopyridin-3-yl)-5-phenyl-3H-imidazo[4,5-b]pyridin-3-yl) benzyl)amino)pyridin-4-yl)-2-hydroxybenzaldehyde

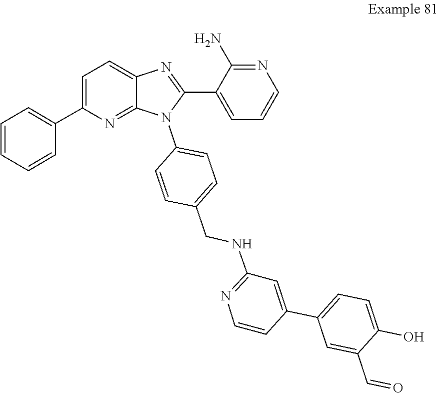 [SMILES: Nc1ncccc1-c1nc2ccc(-c3ccccc3)nc2n1-c1ccc(CNc2cc(-c3ccc(O)c(C=O)c3)ccn2)cc1]

### Synthetic Route:

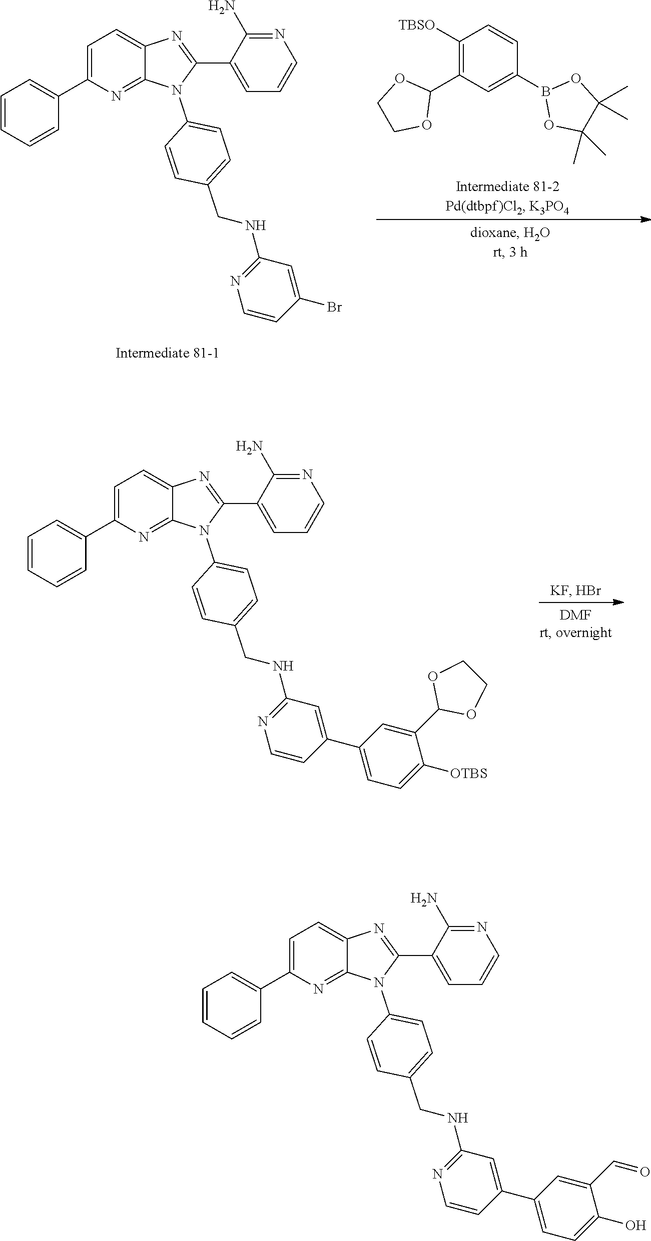

### Step 1: Synthesis of 3-[3-(4-{[(4-{4-[(tert-butyldimethylsilyl)oxy]-3-(1,3-dioxolan-2-yl) phenyl}pyridin-2-yl)amino]methyl}phenyl)-5-phenylimidazo14,5-b]pyridin-2-yl]pyridin-2-amine

To a stirred solution of tert-butyl(2-(1,3-dioxolan-2-yl)-4-(4,4,5,5-tetramethyl-1,3,2-dioxaborolan-2-yl)phenoxy)dimethylsilane (Intermediate 81-2) (300 mg, 0.738 mmol) and 3-[3-(4-{[(4-bromopyridin-2-yl)amino]methyl}phenyl)-5-phenylimidazo[4,5-b]pyridin-2-yl]pyridin-2-amine (Intermediate 81-1) (300 mg, 0.547 mmol) in 1,4-dioxane (7.5 mL) and water (2.5 mL) were added [1,1′-bis(di-tert-butylphosphino)ferrocene]dichloropalladium(II) (40 mg, 0.061 mmol) and tribasic potassium phosphate (240 mg, 1.13 mmol) under nitrogen atmosphere. The resulting mixture was stirred for 3 h at room temperature. The reaction was quenched by the addition of water (10 mL) at room temperature. The resulting mixture was extracted with ethyl acetate (3×10 mL). The combined organic layers were washed with brine (3×10 mL), dried over anhydrous sodium sulfate, filtered, and concentrated under reduced pressure. The residue was purified by reverse-phase flash chromatography using a 5-60% gradient of acetonitrile in water (+0.05% 2,2,2-trifluoroacetic acid) to afford 3-[3-(4-{[(4-{4-[(tert-butyldimethylsilyl)oxy]-3-(1,3-dioxolan-2-yl)phenyl}pyridin-2-yl)amino]methyl}phenyl)-5-phenylimidazo[4,5-b]pyridin-2-yl]pyridin-2-amine (280 mg, 51%) as a yellow solid. MS (ESI) calculated for C₄₄H₄₅N₇O₃Si: 747.34 m/z, found 748.35 [M+H]⁺.

### Step 2: Synthesis of 5-{2-[({4-[2-(2-aminopyridin-3-yl)-5-phenylimidazo[4,5-b]pyridin-3-yl]phenyl}methyl)amino]pyridin-4-yl}-2-hydroxybenzaldehyde (Example 81)

A solution of 3-[3-(4-{[(4-{4-[(tert-butyldimethylsilyl)oxy]-3-(1,3-dioxolan-2-yl)phenyl}pyridin-2-yl)amino]methyl}phenyl)-5-phenylimidazo[4,5-b]pyridin-2-yl]pyridin-2-amine (90 mg, 0.12 mmol), potassium fluoride (20 mg, 0.34 mmol) and hydrogen bromide (0.2 mL, 30% in water) in N,N-dimethylformamide (1 mL) was stirred for 3 h at room temperature. The mixture was purified by preparative HPLC on a XBridge Shield RP18 OBD Column using a 48-64% gradient of acetonitrile in water (+10 mmol/L ammonium bicarbonate) to provide 5-{2-[({4-[2-(2-aminopyridin-3-yl)-5-phenylimidazo[4,5-b]pyridin-3-yl]phenyl}methyl)amino]pyridin-4-yl}-2-hydroxybenzaldehyde (Example 81) (6 mg, 8%) as a yellow solid. MS (ESI) calculated for C₃₆H₂₇N₇O₂: 589.22 m/z, found 590.25 [M+H]⁺. ¹H NMR (300 MHz, DMSO-d₆) δ (ppm): 10.29 (s, 1H), 8.20-8.30 (m, 1H), 7.80-8.15 (m, 7H), 7.50-7.62 (m, 2H), 7.40-7.50 (m, 5H), 7.15-7.29 (m, 1H), 7.00-7.18 (m, 1H), 6.67-6.95 (m, 2H), 6.20-6.45 (m, 1H), 4.60 (s, 2H).

### Intermediate 81-1: N-(4-(2-(2-aminopyridin-3-yl)-5-phenyl-3H-imidazo[4,5-b]pyridin-3-yl)benzyl)-4-bromopyridin-2-amine

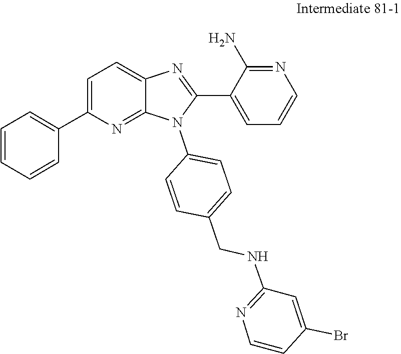 [SMILES: Nc1ncccc1-c1nc2ccc(-c3ccccc3)nc2n1-c1ccc(CNc2cc(Br)ccn2)cc1]

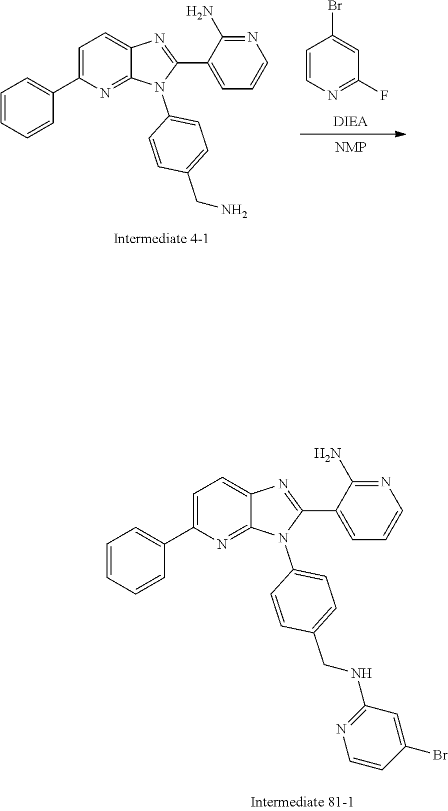

### Step 1: Synthesis of 3-[3-(4-{[(4-bromopyridin-2-yl)amino]methyl}phenyl)-5-phenyl imidazo[4,5-b]pyridin-2-yl]pyridin-2-amine (Intermediate 81-1)

To a stirred solution of 3-{3-[4-(aminomethyl)phenyl]-5-phenylimidazo[4,5-b]pyridin-2-yl}pyridin-2-amine (Intermediate 4-1) (615 mg, 1.57 mmol, 1 equiv) and 4-bromo-2-fluoropyridine (250 mg, 1.42 mmol, 0.9 equiv) in 1-methyl-2-pyrrolidone (10 mL) was added N,N-diisopropylethylamine (1.1 g, 8.5 mmol, 5.4 equiv). The resulting mixture was stirred overnight at 140° C. The reaction was quenched by the addition of water (10 mL) at room temperature. The resulting mixture was extracted with ethyl acetate (3×10 mL). The combined organic layers were washed with brine (3×10 mL), dried over anhydrous sodium sulfate, filtered, and concentrated under reduced pressure. The residue was purified by silica gel column chromatography using a 0-30% gradient of ethyl acetate in petroleum ether to afford 3-[3-(4-{[(4-bromopyridin-2-yl)amino]methyl}phenyl)-5-phenylimidazo[4,5-b]pyridin-2-yl]pyridin-2-amine (Intermediate 81-1) (600 mg, 70%) as a brown oil. MS (ESI) calculated for C₂₉H₂₂BrN₇: 547.11 m/z, found 548.10 [M+H]⁺.

### Intermediate 81-2: (2-(1,3-dioxolan-2-yl)-4-(44,5,5-tetramethyl-1,3,2-dioxaborolan-2-yl)phenoxy)(tert-butyl)dimethylsilane

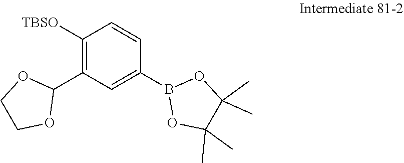 [SMILES: CC1(C)OB(c2ccc(O[Si](C)(C)C(C)(C)C)c(C3OCCO3)c2)OC1(C)C]

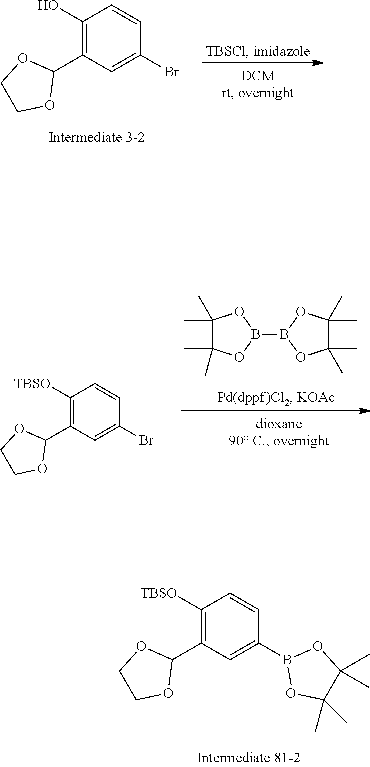

### Step 1: Synthesis of 4-bromo-2-(1,3-dioxolan-2-yl)phenoxy(tert-butyl)dimethylsilane

To a stirred solution of 4-bromo-2-(1,3-dioxolan-2-yl)phenol (Intermediate 3-2) (5.00 g, 20.4 mmol, 1 equiv) and tert-butyldimethylsilyl chloride (3.5 g, 23 mmol, 1.1 equiv) in dichloromethane (50 mL) was added imidazole (2.00 g, 29.4 mmol). The resulting mixture was stirred overnight at room temperature. The reaction was quenched by the addition of water (50 mL). The resulting mixture was extracted with ethyl acetate (3×50 mL). The combined organic layers were washed with brine (3×50 mL), dried over anhydrous sodium sulfate, filtered, and concentrated under reduced pressure. The residue was purified by silica gel column chromatography using a 0-5% gradient of ethyl acetate in petroleum ether to afford 4-bromo-2-(1,3-dioxolan-2-yl)phenoxy(tert-butyl)dimethylsilane (5 g, 68%) as yellow oil. ¹H NMR (300 MHz, DMSO-d₆) δ (ppm): 7.20-7.40 (m, 2H), 6.40-6.80 (m, 1H), 5.60-5.80 (m, 1H), 3.60-3.80 (m, 2H), 3.80-4.00 (m, 2H), 0.70-0.85 (m, 9H).

### Step 2: Synthesis of tert-butyl(2-(1,3-dioxolan-2-yl)-4-(4,4,5,5-tetramethyl-1,3,2-dioxaborolan-2-yl)phenoxy)dimethylsilane (Intermediate 81-2)

To a stirred solution of 4-bromo-2-(1,3-dioxolan-2-yl)phenoxy(tert-butyl)dimethylsilane (1.00 g, 2.78 mmol) and bis(pinacolato)diboron (0.85 g, 3.3 mmol) in 1,4-dioxane (4 mL) were added [1,1′-bis(diphenylphosphino)ferrocene]dichloropalladium(II) (100 mg, 0.137 mmol) and potassium acetate (600 mg, 6.11 mmol) under nitrogen atmosphere. The resulting mixture was stirred overnight at 90° C. The reaction was quenched by the addition of water (10 mL) at room temperature. The mixture was extracted with ethyl acetate (3×10 mL). The combined organic layers were washed with brine (3×10 mL), dried over anhydrous sodium sulfate, filtered, and concentrated under reduced pressure. The residue was purified by silica gel column chromatography using a 0-40% gradient of ethyl acetate in petroleum ether to afford tert-butyl(2-(1,3-dioxolan-2-yl)-4-(4,4,5,5-tetramethyl-1,3,2-dioxaborolan-2-yl)phenoxy)dimethylsilane (Intermediate 81-2) as yellow solid. MS (ESI) calculated for C₂₁H₃₅BO₅Si: 406.23 m/z, found 407.25 [M+H]⁺.

### Example 82: N-(4-(2-(2-aminopyridin-3-yl)-3H-imidazo[4,5-b]pyridin-3-yl)benzyl)-1-(4-formyl-3-hydroxyphenyl)cyclopropane-1-carboxamide

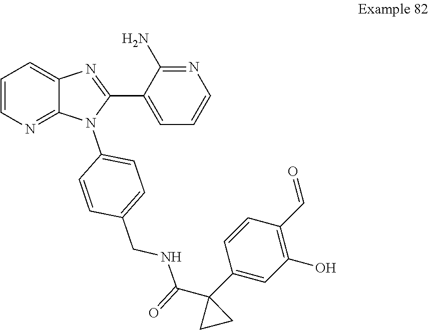 [SMILES: Nc1ncccc1-c1nc2cccnc2n1-c1ccc(CNC(=O)C2(c3ccc(C=O)c(O)c3)CC2)cc1]

Example 82 was prepared in a manner analogous to Example 1 (starting from Step 3) using Intermediate 74-1 in place of the amine starting material and Intermediate 69-1 in place of Intermediate 1-4. MS (ESI) calculated for C₂₉H₂₄N₆O₃: 504.19 m/z, found 505.15 [M+H]⁺. ¹H NMR (300 MHz, DMSO-d₆) δ (ppm): 10.21 (s, 1H), 8.40-8.50 (m, 1H), 8.21-8.42 (m, 1H), 8.00-8.22 (m, 1H), 7.70-7.85 (m, 1H), 7.55-7.70 (m, 1H), 7.38-7.55 (m, 3H), 7.21-7.35 (m, 2H), 6.91-7.12 (m, 2H), 6.70-6.85 (m, 1H), 4.20-4.40 (s, 2H), 1.30-1.55 (m, 2H), 1.02-1.11 (m, 2H).

### Example 83: N-({4-[2-(2-aminopyridin-3-yl)imidazo[4,5-b]pyridin-3-yl]phenyl}methyl)-3-formyl-4-hydroxybenzamide

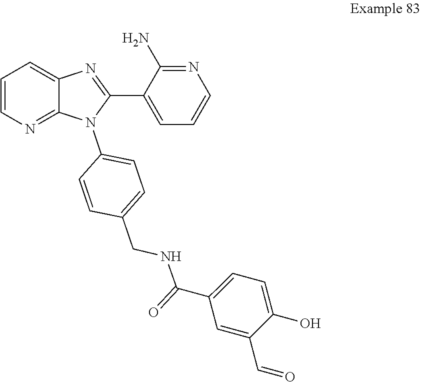 [SMILES: Nc1ncccc1-c1nc2cccnc2n1-c1ccc(CNC(=O)c2ccc(O)c(C=O)c2)cc1]

Example 83 was prepared in a manner analogous to Example 1 (starting from Step 3) using Intermediate 74-1 in place of the amine starting material and Intermediate 3-4 in place of Intermediate 1-4. MS (ESI) calculated for C₂₆H₂₀N₆O₃: 464.16 m/z, found 465.15 [M+H]⁺. ¹H NMR (400 MHz, DMSO-d₆) δ (ppm): 10.28 (s, 1H), 8.30-8.31 (m, 2H), 8.12-8.29 (m, 1H), 7.97-7.98 (m, 2H), 7.35-7.45 (m, 5H), 6.85-7.39 (m, 2H), 6.41-6.44 (m, 1H), 4.39-4.53 (m, 2H).

### Example 84: N-({4-[2-(2-aminopyridin-3-yl)imidazo[4,5-b]pyridin-3-yl]phenyl}methyl)-4-formyl-3-hydroxybenzamide

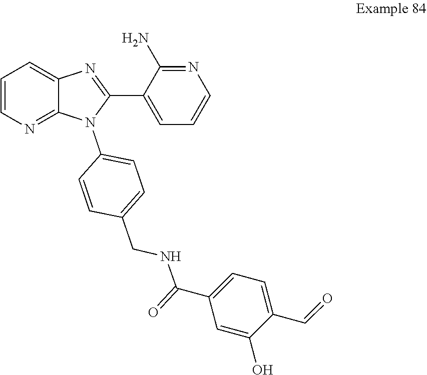 [SMILES: Nc1ncccc1-c1nc2cccnc2n1-c1ccc(CNC(=O)c2ccc(C=O)c(O)c2)cc1]

Example 84 was prepared in a manner analogous to Example 12 using Intermediate 74-1 in place of Intermediate 12-1, 4-formyl-3-hydroxybenzoic acid in place of 3-fluoro-5-formyl-4-hydroxybenzoic acid and PyBOP in place of HATU. MS (ESI) calculated for C₂₆H₂₀N₆O₃: 464.16 m/z, found 465.10 [M+H]⁺. ¹H NMR (400 MHz, DMSO-d₆) δ (ppm): 10.30-10.32 (m, 1H), 8.19-8.31 (m, 2H), 7.99-8.00 (m, 1H), 7.70-7.79 (m, 1H), 7.37-7.56 (m, 7H), 7.37-7.40 (m, 1H), 6.39-6.43 (m, 1H), 4.41-4.54 (m, 2H).

### Example 85: N-({4-[2-(2-aminopyridin-3-yl)-5-phenylimidazo[4,5-b]pyridin-3-yl]phenyl}methyl)-2-fluoro-4-formyl-3-hydroxybenzamide

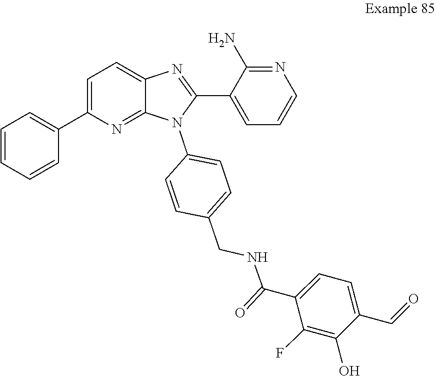 [SMILES: Nc1ncccc1-c1nc2ccc(-c3ccccc3)nc2n1-c1ccc(CNC(=O)c2ccc(C=O)c(O)c2F)cc1]

Example 85 was prepared in a manner analogous to Example 1 (starting from Step 3) using Intermediate 4-1 in place of the amine starting material and Intermediate 85-1 in place of Intermediate 1-4. MS (ESI) calculated for C₃₂H₂₃FN₆O₃: 558.18 m/z, found 559.15 [M+H]⁺. ¹H NMR (300 MHz, DMSO-d₆) δ (ppm): 10.29 (s, 1H), 8.26-8.29 (m, 1H), 7.98-8.04 (m, 4H), 7.38-7.57 (m, 8H), 7.23-7.27 (m, 1H), 7.12-7.16 (m, 1H), 6.44-6.48 (m, 1H), 4.60-4.61 (m, 2H).

### Intermediate 85-1: 4-(1,3-dioxolan-2-yl)-2-fluoro-3-((4-methoxybenzyl)oxy)benzoic acid

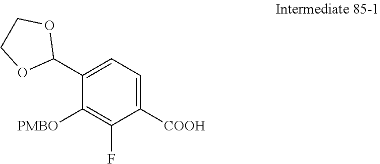

Intermediate 85-1 was prepared in a manner analogous to Intermediate 3-4 using Intermediate 21-1 in place of Intermediate 3-3. MS (ESI) calculated for C₁₈H₁₇FO₆: 348.10 m/z, found 347.05 [M−H]⁻.

### Example 86: 3-(4-(2-(2-aminopyridin-3-yl)-3H-imidazo[4,5-b]pyridin-3-yl)phenyl)-N-(4-formyl-3-hydroxyphenyl)propanamide

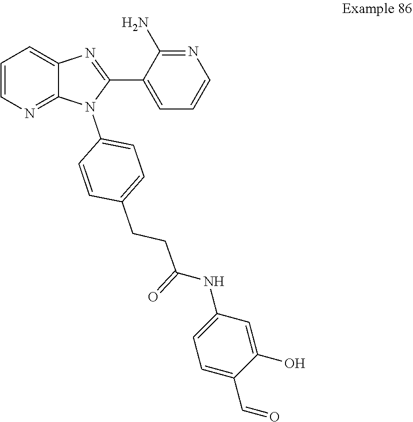 [SMILES: Nc1ncccc1-c1nc2cccnc2n1-c1ccc(CCC(=O)Nc2ccc(C=O)c(O)c2)cc1]

Example 86 was prepared in a manner analogous to Example 1 (starting from Step 3) using Intermediate 86-2 in place of Intermediate 1-4, Intermediate 86-3 in place of the amine starting material and T3P in place of PyBOP. MS (ESI) calculated for C₂₇H₂₂N₆O₃: 478.18 m/z, found 479.10 [M+H]⁺. ¹H NMR (300 MHz, DMSO-d₆) δ (ppm): 10.04 (s, 1H), 8.32-8.34 (m, 1H), 8.18-8.22 (m, 1H), 7.94-7.96 (m, 1H), 7.62-7.64 (m, 1H), 7.51-7.52 (m, 1H), 7.32-7.43 (m, 3H), 7.12-7.19 (m, 2H), 7.03-7.07 (m, 2H), 6.31-6.35 (m, 1H), 2.99-3.01 (m, 2H), 2.75-2.78 (m, 2H).

### Intermediate 86-2: 3-(4-(2-(2-aminopyridin-3-yl)-3H-imidazo[4,5-b]pyridin-3-yl)phenyl) propanoic acid

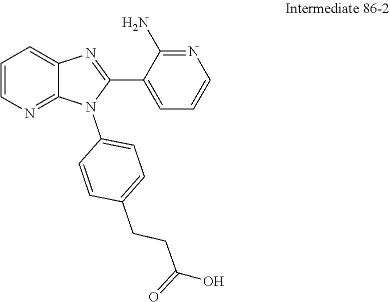 [SMILES: Nc1ncccc1-c1nc2cccnc2n1-c1ccc(CCC(=O)O)cc1]

### Synthetic Route:

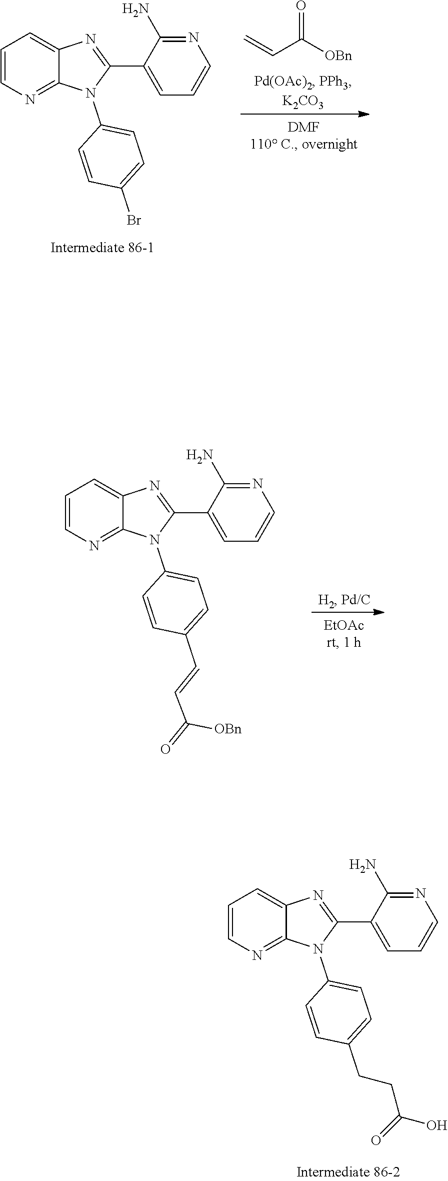

### Step 1: Synthesis of benzyl (2E)-3-{4-[2-(2-aminopyridin-3-yl)imidazo[4,5-b]pyridin-3-yl]phenyl}prop-2-enoate

To a solution of 3-[3-(4-bromophenyl)imidazo[4,5-b]pyridin-2-yl]pyridin-2-amine (Intermediate 86-1) (360 mg, 0.983 mmol, 1 equiv) in N,N-dimethylformamide (5 mL) was added benzyl acrylate (866 mg, 4.92 mmol, 5 equiv), palladium(II) acetate (22 mg, 0.098 mmol, 0.1 equiv), triphenylphosphine (52 mg, 0.20 mmol, 0.20 equiv) and potassium carbonate (271 mg, 1.97 mmol, 2 equiv). The resulting mixture was stirred at 110° C. under nitrogen atmosphere overnight. The mixture was cooled to room temperature, quenched with water (20 mL), and extracted with ethyl acetate (3×20 mL). The organic layers were combined, dried over sodium sulfate, filtered, and concentrated in vacuo. The crude product was purified by reverse-phase flash column chromatography on C18 silica gel using a 20-95% gradient of acetonitrile in water (+0.05% ammonium bicarbonate) with a 10 min hold at 70% acetonitrile to afford benzyl (2E)-3-{4-[2-(2-aminopyridin-3-yl)imidazo[4,5-b]pyridin-3-yl]phenyl}prop-2-enoate (250 mg, 56%) as a yellow solid. MS (ESI) calculated for C₂₇H₂₁N₅O₂: 447.17 m/z, found 448.10 [M+H]⁺.

### Step 2: Synthesis of 3-{4-[2-(2-aminopyridin-3-yl)imidazo[4,5-b]pyridin-3-yl]phenyl}propanoic acid (Intermediate 86-2)

To a mixture of benzyl (2E)-3-{4-[2-(2-aminopyridin-3-yl)imidazo[4,5-b]pyridin-3-yl]phenyl}prop-2-enoate (250 mg, 0.559 mmol, 1 equiv) in ethyl acetate (20 mL) was added 10% palladium on carbon (100 mg, 0.15 equiv) and the mixture was stirred under hydrogen atmosphere for 1 h at room temperature. The resulting mixture was filtered and concentrated in vacuo. The residue obtained was purified by silica gel column chromatography using a 0-30% gradient of ethyl acetate in petroleum ether to afford 3-{4-[2-(2-aminopyridin-3-yl)imidazo[4,5-b]pyridin-3-yl]phenyl}propanoic acid (Intermediate 86-2) (130 mg, 62%) as a yellow solid. MS (ESI) calculated for C₂₀H₁₇N₅O₂: 359.14 m/z, found 360.10 [M+H]⁺.

### Intermediate 86-1: 3-(3-(4-bromophenyl)-3H-imidazo[4,5-b]pyridin-2-yl)pyridin-2-amine

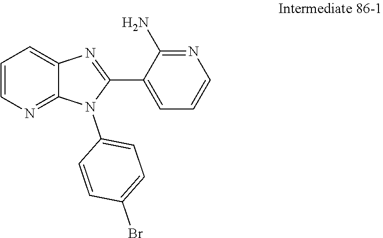 [SMILES: Nc1ncccc1-c1nc2cccnc2n1-c1ccc(Br)cc1]

Intermediate 86-1 was prepared in a manner analogous to Intermediate 11-1 (using Steps 1 and 2 only) using 2-chloro-3-nitropyridine in place of 2-chloro-3-nitro-6-phenylpyridine and 4-bromoaniline in place of tert-butyl 2-(4-aminophenyl)acetate. MS (ESI) calculated for C₁₇H₁₂BrN₅: 365.03 m/z, found 366.00 [M+H]⁺.

### Intermediate 86-3: 4-(1,3-dioxolan-2-yl)-3-((4-methoxybenzyl)oxy)aniline

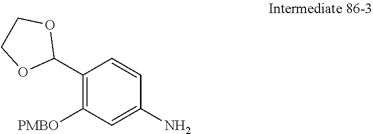

### Synthetic Route:

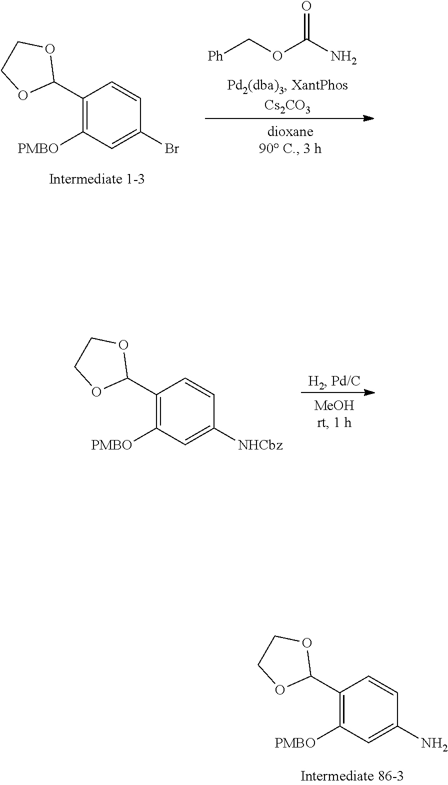

### Step 1: Synthesis of benzyl N-[4-(1,3-dioxolan-2-yl)-3-[(4-methoxyphenyl)methoxy]phenyl]carbamate

To a solution of 2-{4-bromo-2-[(4-methoxyphenyl)methoxy]phenyl}-1,3-dioxolane (Intermediate 1-3) (2.00 g, 5.48 mmol, 1 equiv) in 1,4-dioxane (20 mL) were added O-benzyl carbamate (0.99 g, 6.6 mmol, 1.2 equiv), XantPhos (0.63 g, 1.1 mmol, 0.2 equiv) and tris(dibenzylideneacetone)dipalladium(0) (0.50 g, 0.55 mmol, 0.1 equiv). The resulting mixture was stirred under nitrogen atmosphere at 90° C. for 3 h. The resulting mixture was cooled to room temperature and quenched with water (100 mL). The mixture was extracted with ethyl acetate (3×100 mL). The organic layers were combined, dried over sodium sulfate, filtered, and concentrated in vacuo. The residue obtained was purified by silica gel column chromatography using a 0-20% gradient of ethyl acetate in petroleum ether to afford benzyl N-[4-(1,3-dioxolan-2-yl)-3-[(4-methoxyphenyl)methoxy]phenyl]carbamate (1.4 g, 59%) as a yellow solid. MS (ESI) calculated for C₂₅H₂₅NO₆: 435.17 m/z, found 435.15 [M+H]⁺.

### Step 2: Synthesis of 4-(1,3-dioxolan-2-yl)-3-[(4-methoxyphenyl)methoxy]aniline (Intermediate 86-3)

To a solution of benzyl N-[4-(1,3-dioxolan-2-yl)-3-[(4-methoxyphenyl)methoxy]phenyl]carbamate (1.00 g, 2.30 mmol, 1 equiv) in 10 mL of methanol was added palladium on carbon (10%, 110 mg, 0.05 equiv) and the resulting mixture was stirred at room temperature for 1 h under hydrogen atmosphere. The mixture was then filtered and concentrated in vacuo to afford 4-(1,3-dioxolan-2-yl)-3-[(4-methoxyphenyl)methoxy]aniline (Intermediate 86-3) (500 mg, 72%) as a yellow solid. MS (ESI) calculated for C₁₇H₁₉NO₄: 301.13 m/z, found 302.15 [M+H]⁺.

### Example 87: 4-(2-((4-(2-(2-aminopyridin-3-yl)-5-phenyl-3H-imidazo[4,5-b]pyridin-3-yl)benzyl)amino)pyridin-4-yl)-2-hydroxybenzaldehyde

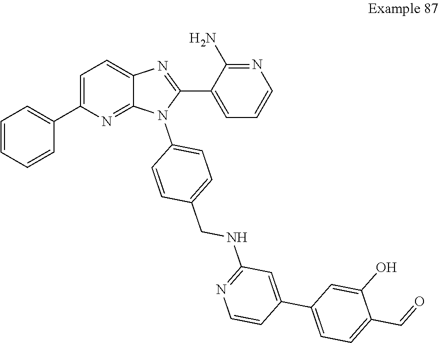 [SMILES: Nc1ncccc1-c1nc2ccc(-c3ccccc3)nc2n1-c1ccc(CNc2cc(-c3ccc(C=O)c(O)c3)ccn2)cc1]

Example 87 was prepared in a manner analogous to Example 81 using Intermediate 87-1 in place of Intermediate 81-2. MS (ESI) calculated for C₃₆H₂₇N₇O₂: 589.22 m/z, found 590.20 [M+H]⁺. ¹H NMR (300 MHz, DMSO-d₆) δ (ppm): 10.31 (s, 1H), 8.20-8.30 (m, 1H), 8.05-8.15 (m, 1H), 7.90-8.05 (m, 4H), 7.70-7.80 (m, 1H), 7.50-7.60 (m, 2H), 7.30-7.50 (m, 5H), 7.10-7.30 (m, 3H), 6.75-6.95 (m, 2H), 6.30-6.40 (m, 1H), 4.66 (s, 2H).

### Intermediate 87-1: (2-(1,3-dioxolan-2-yl)-5-(4,4,5,5-tetramethyl-1,3,2-dioxaborolan-2-yl)phenoxy)(tert-butyl)dimethylsilane

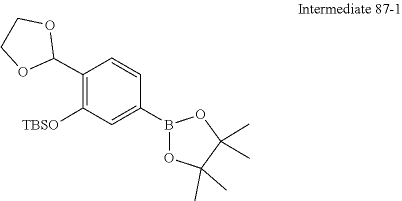 [SMILES: CC1(C)OB(c2ccc(C3OCCO3)c(O[Si](C)(C)C(C)(C)C)c2)OC1(C)C]

Intermediate 87-1 was prepared in a manner analogous to Intermediate 81-2 using Intermediate 1-2 in place of Intermediate 3-2. MS (ESI) calculated for C₂₁H₃₅BO₅Si: 406.23 m/z, found 407.23 [M+H]⁺.

### Example 88: 4-(2-((4-(2-(2-aminopyridin-3-yl)-5-(pyridin-3-yl)-3H-imidazo[4,5-b]pyridin-3-yl)benzyl)amino)ethyl)-3-fluoro-2-hydroxybenzaldehyde

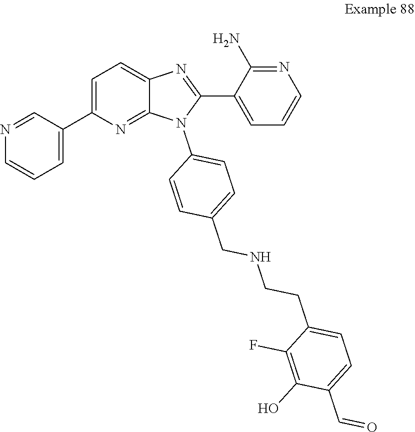 [SMILES: Nc1ncccc1-c1nc2ccc(-c3cccnc3)nc2n1-c1ccc(CNCCc2ccc(C=O)c(O)c2F)cc1]

Example 88 was prepared in a manner analogous to Example 19 using Intermediate 67-1 in place of Intermediate 19-2, Intermediate 88-1 in place of Intermediate 19-3 and dichloromethane/2,2,2-trifluoroacetic acid (15:1) in place of 2,2,2-trifluoroacetic aacid/methanesulfonic acid. MS (ESI) calculated for C₃₂H₂₆FN₇O₂: 559.61 m/z, found 560.25 [M+H]⁺. ¹H NMR (400 MHz, DMSO-d₆) δ (ppm): 10.20 (s, 1H), 9.20-9.22 (m, 1H), 8.58-8.59 (m, 1H), 8.40-8.41 (m, 1H), 8.32-8.38 (m, 1H), 8.09-8.13 (m, 1H), 7.68-8.07 (m, 1H), 7.50-7.66 (m, 4H), 7.48-7.50 (m, 2H), 7.27-7.29 (m, 1H), 6.92-6.95 (m, 1H), 6.42-6.45 (m, 1H), 4.30-4.31 (m, 2H), 3.22-3.26 (m, 2H), 3.04-3.17 (m, 2H). ¹⁹F-NMR (400 MHz, DMSO-d₆) δ (ppm): −140.30.

### Intermediate 88-1: 2-(4-(1,3-dioxolan-2-yl)-2-fluoro-3-((4-methoxybenzyl)oxy)phenyl) acetaldehyde

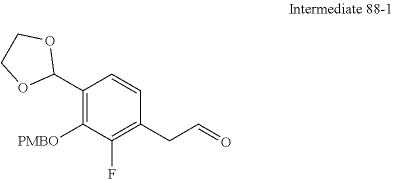

Intermediate 88-1 was prepared in a manner analogous to Intermediate 16-4 using Intermediate 21-1 in place of Intermediate 16-3. MS (ESI) calculated for C₁₉H₁₉FO₅: 346.12 m/z, found 347.15 [M+H]⁺.

### Example 89: N-(3-{2-[({4-[2-(2-aminopyridin-3-yl)imidazo[4,5-b]pyridin-3-yl]phenyl}methyl)amino]ethyl}-2-fluoro-6-formylphenyl)acetamide

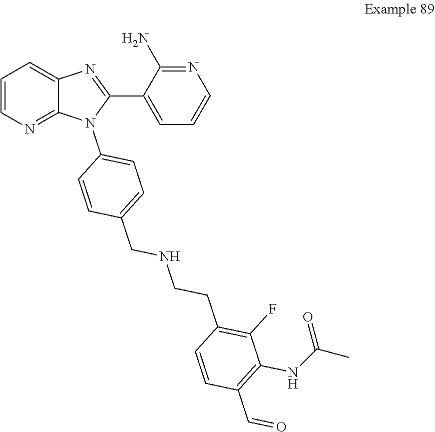 [SMILES: CC(=O)Nc1c(C=O)ccc(CCNCc2ccc(-n3c(-c4cccnc4N)nc4cccnc43)cc2)c1F]

Example 89 was prepared in a manner analogous to Example 19 using Intermediate 74-1 in place of Intermediate 19-2, Intermediate 89-2 in place of Intermediate 19-3, and dichloromethane/2,2,2-trifluoroacetic acid (15:1) in place of 2,2,2-trifluoroacetic acid/methanesulfonic acid. MS (ESI) calculated for C₂₉H₂₆FN₇O₂: 523.21 m/z, found 524.25 [M+H]⁺. ¹H NMR (400 MHz, DMSO-d₆) δ (ppm): 9.85 (s, 1H), 8.31-8.32 (m, 1H), 8.18-8.20 (m, 1H), 7.96-7.97 (m, 1H), 7.52-7.54 (m, 1H), 7.33-7.44 (m, 6H), 7.16-7.18 (m, 1H), 6.33-6.36 (m, 1H), 3.77 (s, 2H), 2.72-2.87 (m, 4H), 2.10 (s, 3H). ¹⁹F NMR (376 MHz, DMSO-d₆) (ppm): −127.32.

### Intermediate 89-2: N-(6-(1,3-dioxolan-2-yl)-2-fluoro-3-(2-oxoethyl)phenyl)acetamide

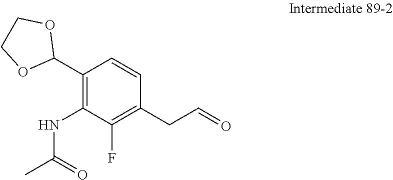 [SMILES: CC(=O)Nc1c(C2OCCO2)ccc(CC=O)c1F]

### Synthetic Route:

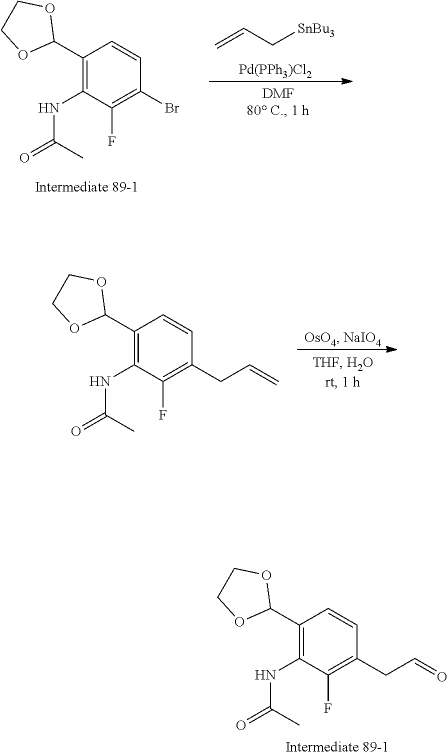

### Step 1: Synthesis of N-[6-(1,3-dioxolan-2-yl)-2-fluoro-3-(prop-2-en-1-yl)phenyl]acetamide

To a solution of N-[3-bromo-6-(1,3-dioxolan-2-yl)-2-fluorophenyl]acetamide (Intermediate 89-1) (1.2 g, 3.9 mmol, 1 equiv) in N,N-dimethylformamide (10 mL) were added tributyl(prop-2-en-1-yl)stannane (2.61 g, 7.89 mmol, 2 equiv) and bis(triphenylphosphine)palladium(II) dichloride (0.28 g, 0.40 mmol, 0.1 equiv) and the resulting mixture was stirred for 1 h at 80° C. The resulting mixture was cooled to room temperature and purified by reverse-phase flash column chromatography on C18 silica gel using a 5-65% gradient of acetonitrile in water (+0.05% ammonium bicarbonate) to afford N-[6-(1,3-dioxolan-2-yl)-2-fluoro-3-(prop-2-en-1-yl)phenyl]acetamide (800 mg, 76%) a yellow oil. MS (ESI) calculated for C₁₄H₁₆FNO₃: 265.11 m/z, found 266.15 [M+H]⁺.

### Step 2: Synthesis of N-[6-(1,3-dioxolan-2-yl)-2-fluoro-3-(2-oxoethyl)phenyl]acetamide (Intermediate 89-2)

To a solution of N-[6-(1,3-dioxolan-2-yl)-2-fluoro-3-(prop-2-en-1-yl)phenyl]acetamide (800 mg, 3.02 mmol, 1 equiv) in tetrahydrofuran (10 mL) and water (10 mL) were added osmium tetroxide (767 mg, 3.02 mmol, 1 equiv) and sodium periodate (1.29 g, 6.03 mmol, 2 equiv) and the resulting mixture was stirred at room temperature for 1 h. The resulting mixture was extracted with ethyl acetate (10 mL×3). The combined organic phases were washed with brine (30 mL×2), dried with sodium sulfate, filtered, and concentrated in vacuo. The residue was purified by silica gel column chromatography to afford N-[6-(1,3-dioxolan-2-yl)-2-fluoro-3-(2-oxoethyl)phenyl]acetamide (Intermediate 89-2) (480 mg, 60%) as a yellow oil. MS (ESI) calculated for C₁₃H₁₄FNO₄: 267.09 m/z, found 268.15 [M+H]⁺.

### Intermediate 89-1: N-(3-bromo-6-(1,3-dioxolan-2-yl)-2-fluorophenyl)acetamide

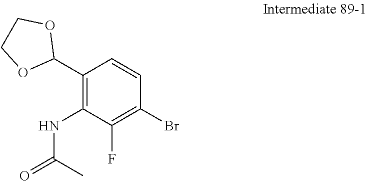 [SMILES: CC(=O)Nc1c(C2OCCO2)ccc(Br)c1F]

### Synthetic Route:

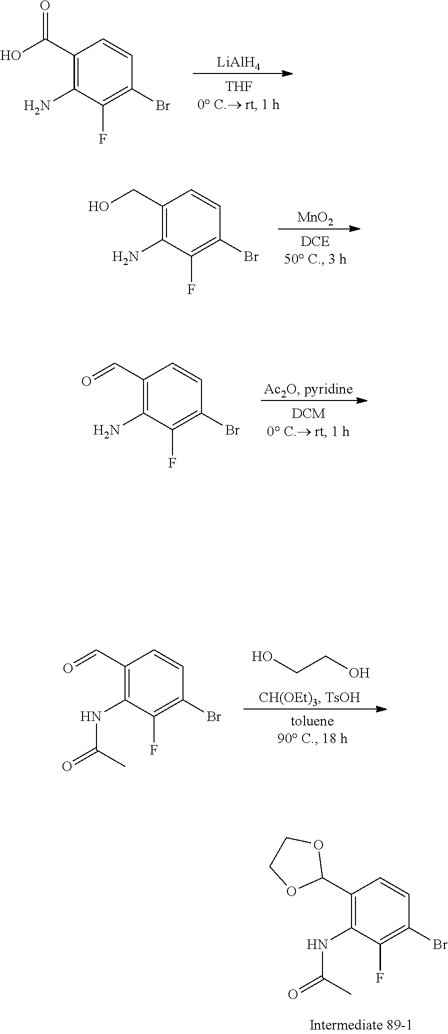

### Step 1: Synthesis of (2-amino-4-bromo-3-fluorophenyl)methanol

To a cooled (0° C.) solution of 2-amino-4-bromo-3-fluorobenzoic acid (10.0 g, 42.7 mmol, 1 equiv) in tetrahydrofuran (500 mL) was added lithium aluminum hydride (3.24 g, 85.5 mmol, 2 equiv) and the resulting mixture was stirred at room temperature for 1 h. The reaction was quenched by sequential addition of water (86 mL), sodium hydroxide (2 M, 86 mL) and water (86 mL). The resulting mixture was filtered and concentrated in vacuo. The residue was purified by silica gel column chromatography to afford (2-amino-4-bromo-3-fluorophenyl)methanol (5 g, 53%) as a yellow oil. MS (ESI) calculated for C₇H₅BrFNO: 218.97 m/z, found 217.85 [M−H]⁻.

### Step 2: Synthesis of 2-amino-4-bromo-3-fluorobenzaldehyde

To a solution of (2-amino-4-bromo-3-fluorophenyl)methanol (5.00 g, 22.7 mmol, 1 equiv) in 1,2-dichloroethane (300 mL) was added manganese (IV) oxide (29.63 g, 340.8 mmol, 15 equiv) and the resulting mixture was stirred for 3 h at 50° C. The resulting mixture was filtered and concentrated in vacuo. The residue was purified by silica gel column chromatography to afford 2-amino-4-bromo-3-fluorobenzaldehyde (3.2 g, 65%) as a yellow oil. MS (ESI) calculated for C₇H₅BrFNO: 216.95 m/z, found 215.90 [M−H]⁻.

### Step 3: Synthesis of N-(3-bromo-2-fluoro-6-formylphenyl)acetamide

To a cooled (0° C.) solution of 2-amino-4-bromo-3-fluorobenzaldehyde (3.00 g, 13.8 mmol, 1 equiv) in dichloromethane (300 mL) were added pyridine (5.44 g, 68.8 mmol, 5 equiv) and acetyl chloride (5.40 g, 68.8 mmol, 5 equiv) and the resulting mixture was stirred at room temperature for 1 h. The reaction was diluted with water (100 mL) and extracted with ethyl acetate (3×200 mL). The combined organic layers were washed with water (3×100 mL), dried over anhydrous sodium sulfate, filtered, and concentrated under reduced pressure. The residue was purified by reverse-phase flash column chromatography on C18 silica gel using a 5-70% gradient of acetonitrile in water (+0.05% ammonium bicarbonate) to afford N-(3-bromo-2-fluoro-6-formylphenyl)acetamide (2.3 g, 60%) as a yellow oil. MS (ESI) calculated for C₉H₇BrFNO₂: 258.96 m/z, found 257.90 [M−H]⁻.

### Step 4: Synthesis of N-[3-bromo-6-(1,3-dioxolan-2-yl)-2-fluorophenyl]acetamide (Intermediate 89-1)

To a solution of N-(3-bromo-2-fluoro-6-formylphenyl)acetamide (2.3 g, 8.8 mmol, 1 equiv), in toluene (30 mL) were added p-toluenesulfonic acid (0.15 g, 0.88 mmol, 0.1 equiv), triethyl orthoformate (6.55 g, 44.2 mmol, 5 equiv) and ethylene glycol (1.65 g, 26.5 mmol, 3 equiv) and the resulting mixture was stirred for 18 h at 90° C. The resulting mixture was cooled to room temperature, concentrated in vacuo, and purified by silica gel column chromatography to afford N-[3-bromo-6-(1,3-dioxolan-2-yl)-2-fluorophenyl]acetamide (Intermediate 89-1) (1.46 g, 54%) as a white solid. MS (ESI) calculated for C₁₁H₁₁BrFNO₃: 302.99 m/z, found 303.95 [M+H]⁺.

### Example 90: N-(3-{2-[({4-[2-(2-aminopyridin-3-yl)-5-phenylimidazo[4,5-b]pyridin-3-yl]phenyl}methyl)amino]ethyl}-2-fluoro-6-formylphenyl)acetamide

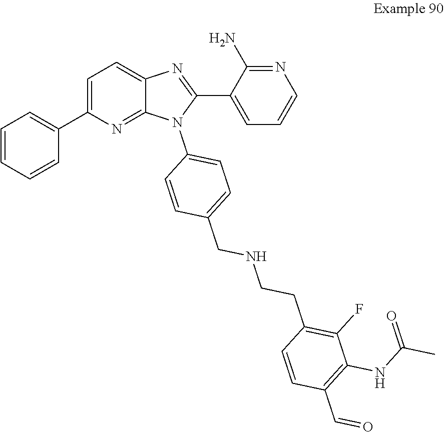 [SMILES: CC(=O)Nc1c(C=O)ccc(CCNCc2ccc(-n3c(-c4cccnc4N)nc4ccc(-c5ccccc5)nc43)cc2)c1F]

Example 90 was prepared in a manner analogous to Example 19 using Intermediate 4-1 in place of Intermediate 19-2, Intermediate 89-2 in place of Intermediate 19-3, and dichloromethane/2,2,2-trifluoroacetic acid (6:1) in place of 2,2,2-trifluoroacetic acid/methanesulfonic acid. MS (ESI) calculated for C₃₅H₃₀FN₇O₂: 599.24 m/z, found 600.20 [M+H]⁺. ¹H NMR (400 MHz, DMSO-d₆) δ (ppm): 9.90 (s, 1H), 8.26-8.32 (m, 1H), 7.97-8.06 (m, 4H), 7.55-7.68 (m, 5H), 7.36-7.50 (m, 4H), 7.20-7.26 (m, 1H), 6.40 (dd, J=7.6, 4.9 Hz, 1H), 4.26 (s, 2H), 3.18-3.25 (m, 2H), 3.04-3.12 (m, 2H), 2.14 (s, 3H). ¹⁹F NMR (376 MHz, DMSO-d₆) (ppm): −126.27.

### Example 91: 2-(3-acetamido-4-formylphenyl)-N-(4-(2-(2-aminopyridin-3-yl)-3H-imidazo[4,5-b]pyridin-3-yl)benzyl)acetamide

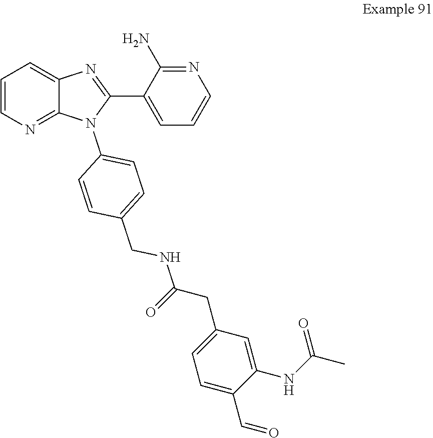 [SMILES: CC(=O)Nc1cc(CC(=O)NCc2ccc(-n3c(-c4cccnc4N)nc4cccnc43)cc2)ccc1C=O]

Example 91 was prepared in a manner analogous to Example 1 (starting from Step 3) using Intermediate 74-1 in place of the amine starting material and Intermediate 91-1 in place of Intermediate 1-4. MS (ESI) calculated for C₂₉H₂₅N₇O₂: 519.20 m/z, found 520.20 [M+H]⁺. ¹H NMR (400 MHz, DMSO-d₆) δ (ppm): 10.73 (s, 1H), 9.93 (s, 1H), 8.77-8.76 (m, 1H), 8.31-8.32 (m, 1H), 8.19-8.21 (m, 1H), 8.10-8.14 (m, 1H), 7.99-8.00 (m, 1H), 7.78-7.80 (m, 1H), 7.37-7.40 (m, 5H), 7.20-7.25 (m, 2H), 7.02-7.21 (m, 2H), 6.40-6.43 (m, 1H), 4.36-4.38 (m, 2H), 3.56-3.61 (m, 2H), 2.07-2.14 (m, 3H).

### Intermediate 91-1: 2-(3-acetamido-4-(1,3-dioxolan-2-yl)phenyl)acetic acid

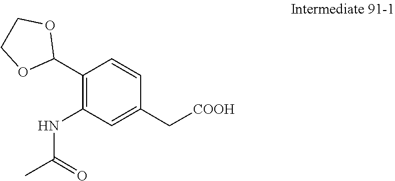 [SMILES: CC(=O)Nc1cc(CC(=O)O)ccc1C1OCCO1]

### Synthetic Route:

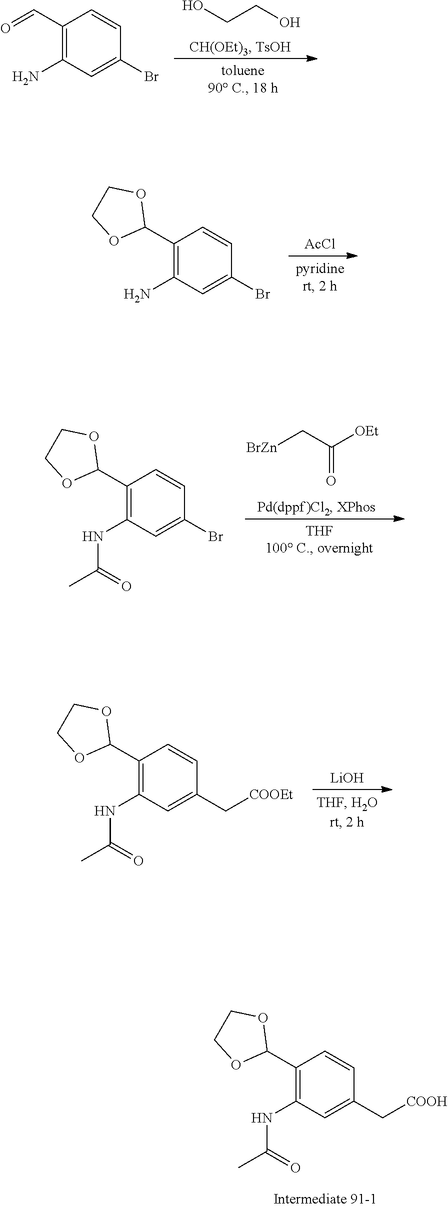

### Step 1: Synthesis of 5-bromo-2-(1,3-dioxolan-2-yl)aniline

To a solution of 2-amino-4-bromobenzaldehyde (4.95 g, 24.9 mmol, 1 equiv) in toluene (100 mL) was added p-toluenesulfonic acid (0.43 g, 2.5 mmol, 0.1 equiv), ethylene glycol (7.72 g, 124 mmol, 5 equiv) and triethyl orthoformate (11.06 g, 74.62 mmol, 3 equiv). The obtained solution was stirred at room temperature for 10 min then for 18 h at 90° C. The resulting mixture was cooled to 0° C. and quenched by the addition of saturated aqueous ammonium chloride (80 mL). The mixture was extracted with ethyl acetate (100 mL×3). The combined organic layers were washed with water (100 mL×3), dried over anhydrous sodium sulfate, filtered, and concentrated under reduced pressure. The residue was purified by silica gel column chromatography using a 0-7% gradient of ethyl acetate in petroleum ether to provide 5-bromo-2-(1,3-dioxolan-2-yl)aniline (3 g, 49%) as a light-yellow oil. MS (ESI) calculated for C₉H₁₀BrNO₂: 242.99 m/z, found 244.10 [M+H]⁺.

### Step 2: Synthesis of N-(5-bromo-2-(1,3-dioxolan-2-yl)phenyl)acetamide

A solution of 5-bromo-2-(1,3-dioxolan-2-yl)aniline (3.00 g, 12.3 mmol, 1 equiv) in pyridine was sparged with argon for 15 min under dry conditions. Acetyl chloride (3.86 g, 49.2 mmol, 4 equiv) was added and the resulting mixture stirred for 2 h at room temperature. The residue was purified by silica gel column chromatography to afford N-(5-bromo-2-(1,3-dioxolan-2-yl)phenyl)acetamide (2 g, 57%) as a yellow oil. MS (ESI) calculated for C₁₁H₁₂BrNO₃: 285.00 m/z, found 286.05, 288.05 [M+H, M+H+2]⁺.

### Step 3: Synthesis of ethyl 2-(3-acetamido-4-(1,3-dioxolan-2-yl)phenyl)acetate

To a solution of N-[5-bromo-2-(1,3-dioxolan-2-yl)phenyl]acetamide (2.0 g, 7.0 mmol, 1 equiv) and (2-ethoxy-2-oxoethyl)zinc(II) bromide (20 mL, 0.5 M in tetrahydrofuran, 1.5 equiv) in tetrahydrofuran (5 mL) were added [1,1′-bis(diphenylphosphino)ferrocene]dichloropalladium(II) (0.50 g, 0.70 mmol, 0.1 equiv) and XPhos (0.65 g, 1.4 mmol, 0.2 equiv). After stirring overnight at 100° C. under a nitrogen atmosphere, the mixture was cooled to 0° C. and quenched with water (10 mL). The resulting mixture was extracted with ethyl acetate (20 mL×3). The combined organic layers were washed with water (10 mL×3), dried over anhydrous sodium sulfate, filtered, and concentrated under reduced pressure. The residue was purified by silica gel column chromatography using a 0-60% gradient of ethyl acetate in petroleum ether to provide ethyl 2-(3-acetamido-4-(1,3-dioxolan-2-yl)phenyl)acetate (1.0 g, 49%) as a light-yellow solid. MS (ESI) calculated for C₁₅H₁₉NO₅: 293.13 m/z, found 294.10 [M+H]⁺.

### Step 4: Synthesis of 2-(3-acetamido-4-(1,3-dioxolan-2-yl)phenyl)acetic acid (Intermediate 91-1)

To a solution of ethyl 2-(3-acetamido-4-(1,3-dioxolan-2-yl)phenyl)acetate (1.0 g, 3.4 mmol, 1 equiv) in tetrahydrofuran (10 mL) and water (10 mL) was added lithium hydroxide (5.0 mL, 2 M in water, 3 equiv). The resulting mixture was stirred at room temperature for 1 h then concentrated in vacuo and purified by reverse-phase flash column chromatography on C₁₈ silica gel using a 20-95% gradient of acetonitrile in water (+0.05% ammonium bicarbonate) with a 10 min hold at 70% acetonitrile to provide 2-(3-acetamido-4-(1,3-dioxolan-2-yl)phenyl)acetic acid (Intermediate 91-1) (600 mg, 66%). MS (ESI) calculated for C₁₃H₁₅NO₅: 265.10 m/z, found 266.10 [M+H]⁺.

### Example 92: 5-(2-{[(1S)-1-{4-[2-(2-aminopyridin-3-yl)-5-phenylimidazo[4,5-b]pyridin-3-yl]phenyl}ethyl]amino}ethyl)-2-hydroxybenzaldehyde

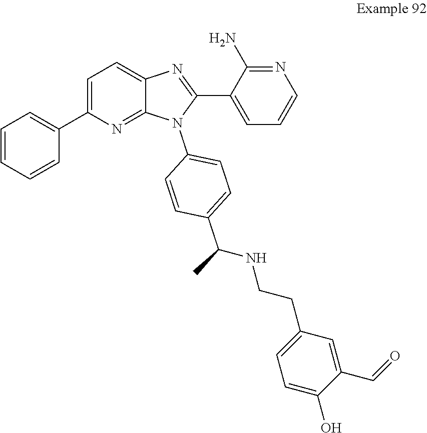 [SMILES: C[C@H](NCCc1ccc(O)c(C=O)c1)c1ccc(-n2c(-c3cccnc3N)nc3ccc(-c4ccccc4)nc32)cc1]

Example 92 was prepared in a manner analogous to Example 19 using Intermediate 92-2 in place of Intermediate 19-2, Intermediate 92-3 in place of Intermediate 19-3 and dichloromethane/2,2,2-trifluoroacetic acid (5:1) in place of 2,2,2-trifluoroacetic acid/methanesulfonic acid. MS (ESI) calculated for C₃₄H₃₀N₆O₂: 554.24 m/z, found 555.30 [M+H]⁺. ¹H NMR (300 MHz, DMSO-d₆) δ (ppm): 10.22 (s, 1H), 8.23-8.29 (m, 1H), 7.95-8.02 (m, 5H), 7.56-7.57 (m, 2H), 7.64-7.66 (m, 1H), 7.33-7.48 (m, 8H), 7.09-7.12 (m, 1H), 6.21-6.25 (m, 1H), 4.04-4.06 (m, 1H), 2.71-2.74 (m, 3H), 2.63 (s, 1H), 1.41-1.49 (m, 3H).

### Intermediate 92-2: (S)-3-(3-(4-(1-aminoethyl)phenyl)-5-phenyl-3H-imidazo[4,5-b]pyridin-2-yl)pyridin-2-amine

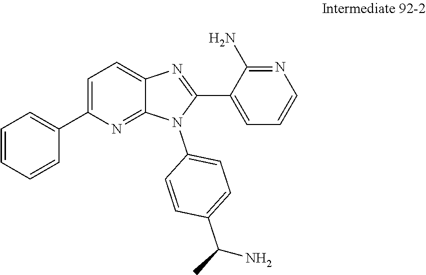 [SMILES: C[C@H](N)c1ccc(-n2c(-c3cccnc3N)nc3ccc(-c4ccccc4)nc32)cc1]

Intermediate 92-2 was prepared in a manner analogous to Intermediate 11-1 using Intermediate 92-1 in place of tert-butyl 2-(4-aminophenyl)acetate. MS (ESI) calculated for C₂₅H₂₂N₆: 406.19 m/z, found 407.10 [M+H]⁺.

### Intermediate 92-1: tert-butyl (S)-(1-(4-aminophenyl)ethyl)carbamate

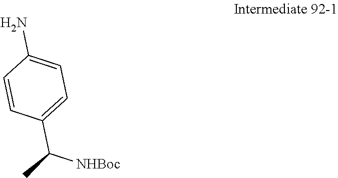 [SMILES: C[C@H](NC(=O)OC(C)(C)C)c1ccc(N)cc1]

### Synthetic Route:

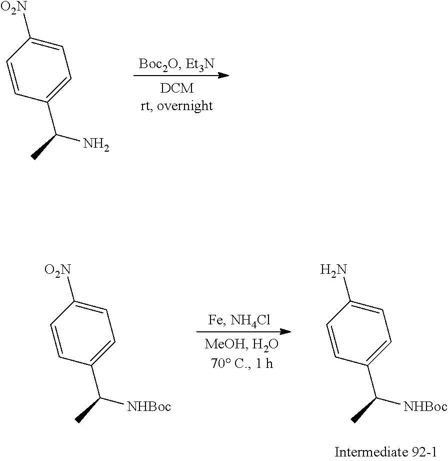

### Step 1: Synthesis of tert-butyl (S)-(1-(4-nitrophenyl)ethyl)carbamate

To a solution of (S)-1-(4-nitrophenyl)ethan-1-amine (5.00 g, 30.1 mmol, 1 equiv) in dichloromethane (100 mL) were added di-tert-butyl dicarbonate (13.13 g, 60.18 mmol, 2 equiv) and triethylamine (9.13 g, 90.3 mmol, 3 equiv). The resulting mixture was stirred at room temperature overnight. The reaction was quenched with water (100 mL). The resulting mixture was extracted with dichloromethane (3×100 mL). The organic layers were combined, dried over sodium sulfate, filtered, and concentrated in vacuo. The residue obtained was purified by silica gel column chromatography using a 0-20% gradient of ethyl acetate in petroleum ether to afford tert-butyl (S)-(1-(4-nitrophenyl)ethyl)carbamate (6 g, 75%) as a white solid. MS (ESI) calculated for C₁₃H₁₈N₂O₄: 266.13 m/z, found 267.15 [M+H]⁺.

### Step 2: Synthesis of tert-butyl N-[(1S)-1-(4-aminophenyl)ethyl]carbamate (Intermediate 92-1)

To a solution of tert-butyl N-[(1S)-1-(4-nitrophenyl)ethyl]carbamate (1.00 g, 3.756 mmol, 1 equiv) in methanol (10 mL) and water (10 mL) were added iron (1.68 g, 30.0 mmol, 8 equiv) and ammonium chloride (1.00 g, 18.8 mmol, 5 equiv). The resulting mixture was stirred at 70° C. for 1 h. The resulting mixture was cooled to room temperature and quenched with water (50 mL). The mixture was extracted with ethyl acetate (3×50 mL). The organic layers were combined, dried over sodium sulfate, filtered, and concentrated in vacuo. The residue obtained was purified by silica gel column chromatography using a 0-10% gradient of ethyl acetate in petroleum ether to afford tert-butyl N-[(1S)-1-(4-aminophenyl)ethyl]carbamate (Intermediate 92-1) (850 mg, 96%) as a yellow solid. MS (ESI) calculated for C₁₃H₂₀N₂O₂: 236.15 m/z, found 237.25 [M+H]⁺.

### Intermediate 92-3: 2-[3-(1,3-dioxolan-2-yl)-4-[(4-methoxyphenyl)methoxy]phenyl]acetaldehyde

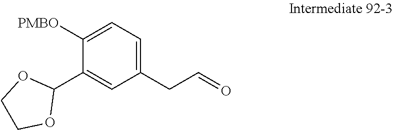

### Synthetic Route:

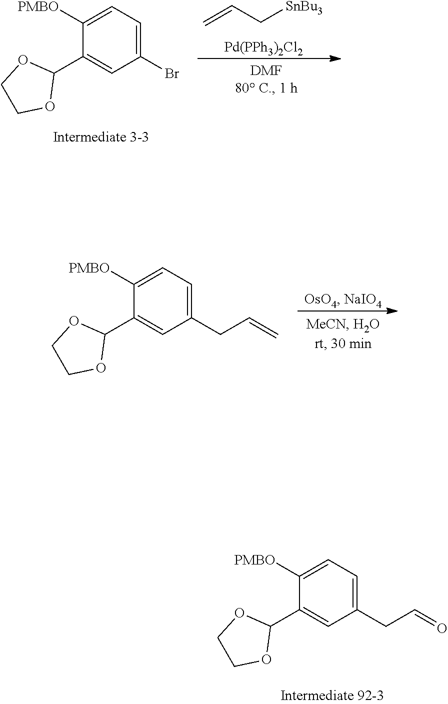

### Step 1: Synthesis of 2-(5-allyl-2-((4-methoxybenzyl)oxy)phenyl)-1,3-dioxolane

To a solution of 2-(5-bromo-2-((4-methoxybenzyl)oxy)phenyl)-1,3-dioxolane (Intermediate 3-3) (3 g, 8.2 mmol, 1 equiv) and tributyl(prop-2-en-1-yl)stannane (5.44 g, 16.4 mmol, 2 equiv) in N,N-dimethylformamide (24 mL) was added bis(triphenylphosphine)palladium(II) dichloride (0.58 g, 0.82 mmol, 0.1 equiv). After stirring for 1 h at 80° C. under a nitrogen atmosphere, the mixture was concentrated under reduced pressure. The residue was purified by reverse-phase flash chromatography on C18 silica gel using a 10 to 40% gradient of acetonitrile to provide 2-(5-allyl-2-((4-methoxybenzyl)oxy)phenyl)-1,3-dioxolane (2.4 g, 90%) as colorless oil. MS (ESI) calculated for C₂₀H₂₂O₄: 326.15 m/z, found 327.20 [M+H]⁺.

### Step 2: Synthesis of 2-(3-(1,3-dioxolan-2-yl)-4-((4-methoxybenzyl)oxy)phenyl)acetaldehyde (Intermediate 92-3)

To a solution of 2-(5-allyl-2-((4-methoxybenzyl)oxy)phenyl)-1,3-dioxolane (0.5 g, 1.53 mmol, 1 equiv) in acetonitrile (3 mL) and water (3 mL) were added osmium tetroxide (1.17 g, 4.60 mmol, 3 equiv) and sodium periodate (0.98 g, 4.60 mmol, 3 equiv). After stirring for 30 min at room temperature, the reaction was quenched by the addition of water. The resulting mixture was extracted with ethyl acetate (20 mL×3). The combined organic layers were washed with water (20 mL×3), dried over anhydrous sodium sulfate, filtered, and concentrated in vacuo. The residue was purified by silica gel column chromatography using a gradient of 0-25% ethyl acetate in petroleum ether to provide 2-(3-(1,3-dioxolan-2-yl)-4-((4-methoxybenzyl)oxy)phenyl) acetaldehyde (Intermediate 92-3) (0.15 g, 30%) as a light-yellow oil. MS (ESI) calculated for C₁₉H²⁰O₅: 328.13 m/z, found 329.15 [M+H]⁺.

### Example 93: N-({4-[2-(2-aminopyridin-3-yl)imidazo[4,5-b]pyridin-3-yl]phenyl}methyl)-2-(2-fluoro-4-formyl-3-hydroxyphenyl)acetamide

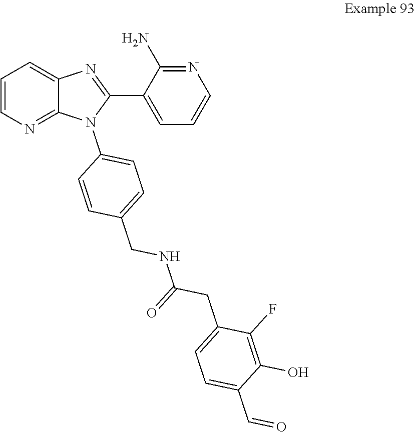 [SMILES: Nc1ncccc1-c1nc2cccnc2n1-c1ccc(CNC(=O)Cc2ccc(C=O)c(O)c2F)cc1]

Example 93 was prepared in a manner analogous to Example 1 (starting from Step 3) using Intermediate 74-1 in place of the amine starting material and Intermediate 21-2 in place of Intermediate 1-4. MS (ESI) calculated for C₂₇H₂₁FN₆O₃: 496.17 m/z, found 497.15 [M+H]⁺. ¹H NMR (400 MHz, DMSO-d₆) δ (ppm): 10.15 (s, 1H), 8.30-8.32 (m, 1H), 8.21-8.29 (m, 1H), 7.95-7.97 (m, 1H), 7.39-7.43 (m, 6H), 7.22-7.24 (m, 1H), 6.86-6.90 (m, 1H), 6.40-6.44 (m, 1H), 4.35-4.36 (m, 2H), 3.63-3.72 (m, 2H). ¹⁹F-NMR (400 MHz, DMSO-d₆) δ (ppm): −139.35.

### Example 94: N-({4-[2-(2-aminopyridin-3-yl)-5-phenylimidazo[4,5-b]pyridin-3-yl]phenyl}methyl)-2-(2-fluoro-4-formyl-3-hydroxyphenyl)acetamide

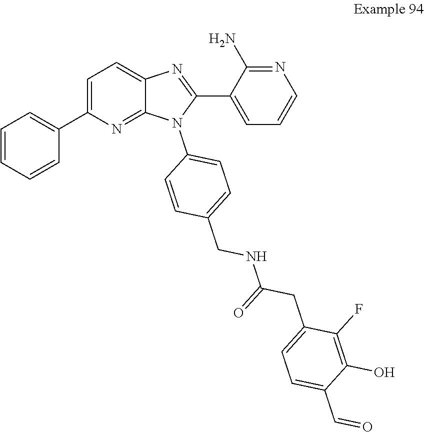 [SMILES: Nc1ncccc1-c1nc2ccc(-c3ccccc3)nc2n1-c1ccc(CNC(=O)Cc2ccc(C=O)c(O)c2F)cc1]

Example 94 was prepared in a manner analogous to Example 1 (starting from Step 3) using Intermediate 4-1 in place of the amine starting material and Intermediate 21-2 in place of Intermediate 1-4. MS (ESI) calculated for C₃₃H₂₅FN₆O₃: 572.20 m/z, found 573.20 [M+H]⁺. ¹H NMR (400 MHz, DMSO-d₆) δ (ppm): 10.15 (s, 1H), 8.31-8.33 (m, 1H), 7.99-8.04 (m, 4H), 7.79-7.99 (m, 1H), 7.39-7.77 (m, 8H), 6.82-6.95 (m, 2H), 4.20-4.60 (m, 2H), 3.62-3.74 (m, 2H). ¹⁹F-NMR (400 MHz, DMSO-d₆) δ (ppm): −139.30.

### Example 95: 4-(2-((4-(2-(2-aminopyridin-3-yl)-3H-imidazo[4,5-b]pyridin-3-yl)benzyl)amino)ethyl)-3-fluoro-2-hydroxybenzaldehyde

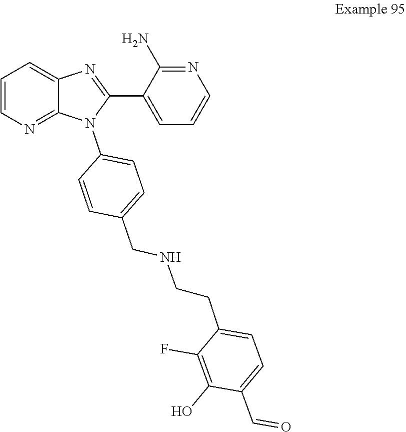 [SMILES: Nc1ncccc1-c1nc2cccnc2n1-c1ccc(CNCCc2ccc(C=O)c(O)c2F)cc1]

Example 95 was prepared in a manner analogous to Example 19 using Intermediate 74-1 in place of Intermediate 19-2, Intermediate 88-1 in place of Intermediate 19-3 and dichloromethane/2,2,2-trifluoroacetic acid (15:1) in place of 2,2,2-trifluoroacetic acid/methanesulfonic acid. MS (ESI) calculated for C₂₇H₂₃FN₆O₂: 482.19 m/z, found 483.20 [M+H]⁺. ¹H-NMR (300 MHz, DMSO-d₆) δ (ppm): 10.12-10.19 (m, 1H), 8.30-8.34 (m, 1H), 8.20-8.22 (m, 2H), 7.96-8.00 (m, 1H), 7.52-7.55 (m, 2H), 7.40-7.43 (m, 5H), 7.18-7.23 (m, 1H), 6.83-6.87 (m, 1H), 6.36-6.41 (m, 1H), 4.00-4.01 (m, 2H), 2.84-2.93 (m, 4H).

### Example 96: N-(4-(2-(2-aminopyridin-3-yl)-3H-imidazo[4,5-b]pyridin-3-yl)benzyl)-2-(4-formyl-3-hydroxyphenyl)acetamide

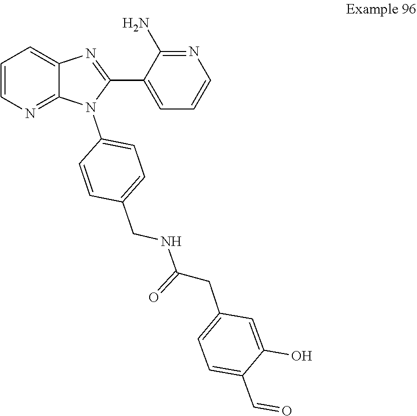 [SMILES: Nc1ncccc1-c1nc2cccnc2n1-c1ccc(CNC(=O)Cc2ccc(C=O)c(O)c2)cc1]

Example 96 was prepared in a manner analogous to Example 1 (starting from Step 3) using Intermediate 74-1 in place of the amine starting material. MS (ESI) calculated for C₂₇H₂₂N₆O₃: 478.18 m/z, found 479.15 [M+H]⁺. ¹H NMR (300 MHz, DMSO-d₆) δ (ppm): 10.19 (s, 1H), 8.33-8.35 (m, 1H), 8.14-8.23 (m, 1H), 8.00-8.02 (m, 1H), 7.60-7.62 (m, 1H), 7.33-7.43 (m, 6H), 6.89-6.96 (m, 2H), 6.46-6.51 (m, 1H), 4.30-4.36 (m, 2H), 3.46-3.54 (m, 2H).

### Example 97: 4-{2-[({4-[2-(2-aminopyridin-3-yl)-5-phenylimidazo[4,5-b]pyridin-3-yl]phenyl}methyl)amino]ethyl}-2-methoxybenzaldehyde

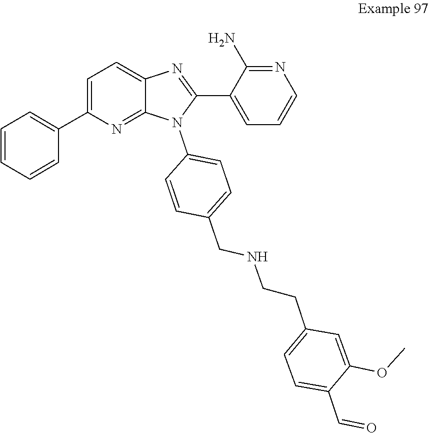 [SMILES: COc1cc(CCNCc2ccc(-n3c(-c4cccnc4N)nc4ccc(-c5ccccc5)nc43)cc2)ccc1C=O]

Example 97 was prepared in a manner analogous to Example 19 using Intermediate 4-1 in place of Intermediate 19-2, Intermediate 97-2 in place of Intermediate 19-3 and dichloromethane/2,2,2-trifluoroacetic acid (10:1) in place of 2,2,2-trifluoroacetic acid/methanesulfonic acid. MS (ESI) calculated for C₃₄H₃₀N₆O₂: 554.24 m/z, found 555.25 [M+H]⁺. ¹H NMR (400 MHz, DMSO-d₆) δ (ppm): 10.25 (s, 1H), 8.24-8.26 (m, 1H), 7.99-8.01 (m, 4H), 7.97-7.98 (m, 1H), 7.50-7.61 (m, 7H), 7.19-7.36 (m, 1H), 7.08-7.16 (m, 1H), 7.69-7.08 (m, 1H), 6.33-6.36 (m, 1H), 3.86-3.87 (m, 3H), 3.54-3.57 (m, 2H), 2.76-2.85 (m, 4H).

### Intermediate 97-2: 2-(4-(1,3-dioxolan-2-yl)-3-methoxyphenyl)acetaldehyde

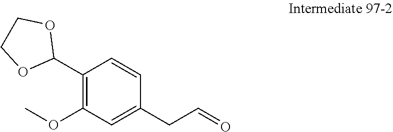 [SMILES: COc1cc(CC=O)ccc1C1OCCO1]

Intermediate 97-2 was prepared in a manner analogous to Intermediate 16-4 using Intermediate 97-1 in place of Intermediate 16-3. MS (ESI) calculated for C₁₂H₁₄O₄: 222.09 m/z, found 223.15 [M+H]⁺.

### Intermediate 97-1: 2-(4-bromo-2-methoxyphenyl)-1,3-dioxolane

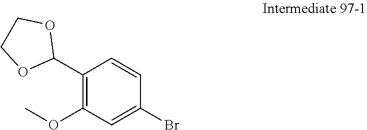

Intermediate 97-1 was prepared in a manner analogous to Intermediate 1-2 using 4-bromo-2-methoxybenzaldehyde in place of 4-bromo-2-hydroxybenzaldehyde. MS (ESI) calculated for C₁₀H₁₁BrO₃: 257.99 m/z, found 259.05 [M+H]⁺.

### Example 98: N-(5-{2-[({4-[2-(2-aminopyridin-3-yl)imidazo[4,5-b]pyridin-3-yl]phenyl}methyl)amino]ethyl}-2-formylphenyl)acetamide

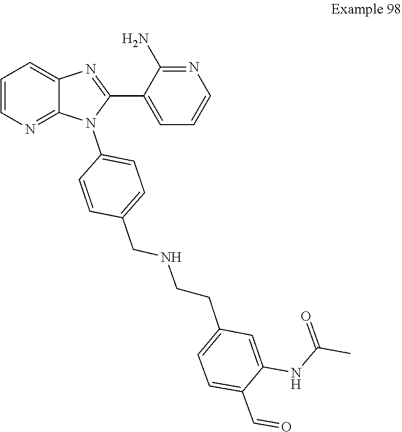 [SMILES: CC(=O)Nc1cc(CCNCc2ccc(-n3c(-c4cccnc4N)nc4cccnc43)cc2)ccc1C=O]

Example 98 was prepared in a manner analogous to Example 19 using Intermediate 74-1 in place of Intermediate 19-2, Intermediate 98-3 in place of Intermediate 19-3 and dichloromethane/2,2,2-trifluoroacetic acid (10:1) in place of 2,2,2-trifluoroacetic acid/methanesulfonic acid. MS (ESI) calculated for C₂₉H₂₇N₇O₂: 505.22 m/z, found 506.15 [M+H]⁺. ¹H NMR (300 MHz, DMSO-d₆) δ (ppm): 9.94 (s, 1H), 8.32-8.40 (m, 1H), 8.19-8.28 (m, 1H), 8.08-8.15 (m, 1H), 7.96-8.04 (m, 1H), 7.81-7.89 (m, 1H), 7.59-7.68 (m, 2H), 7.49-7.57 (m, 2H), 7.39-7.48 (m, 2H), 7.18-7.27 (m, 1H), 6.47-6.59 (m, 1H), 4.27 (s, 2H), 3.19-3.27 (m, 2H), 3.00-3.08 (m, 2H), 2.15 (s, 3H).

### Intermediate 98-3: N-(2-(1,3-dioxolan-2-yl)-5-(2-oxoethyl)phenyl)acetamide

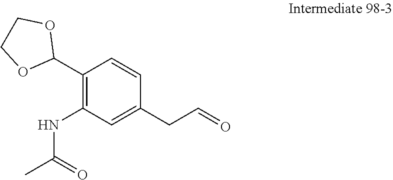 [SMILES: CC(=O)Nc1cc(CC=O)ccc1C1OCCO1]

Intermediate 98-3 was prepared in a manner analogous to Intermediate 16-4 using Intermediate 98-2 in place of Intermediate 16-3. MS (ESI) calculated for C₁₃H₁₅NO₄: 249.10 m/z, found 250.10 [M+H]⁺.

### Intermediate 98-2: N-(5-bromo-2-(1,3-dioxolan-2-yl)phenyl)acetamide

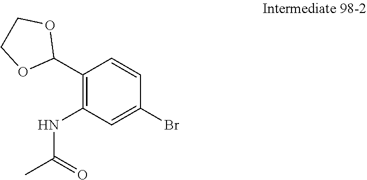 [SMILES: CC(=O)Nc1cc(Br)ccc1C1OCCO1]

### Synthetic Route:

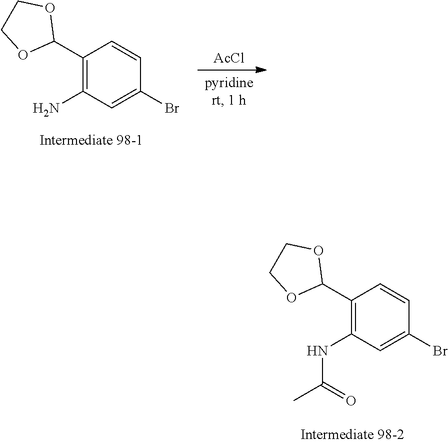 [SMILES: "AcCl" ; Nc1cc(Br)ccc1C1OCCO1 ; CC(=O)Nc1cc(Br)ccc1C1OCCO1]

Step 1: Synthesis of N-[5-bromo-2-(1,3-dioxolan-2-yl)phenyl]acetamide (Intermediate 98-1)

To a solution of 5-bromo-2-(1,3-dioxolan-2-yl)aniline (Intermediate 98-1) (9.00 g, 36.9 mmol, 1 equiv) in pyridine (180 mL) was added acetyl chloride (11.58 g, 147.5 mmol, 4 equiv) and the resulting mixture was stirred at room temperature for 1 h. The resulting mixture was concentrated in vacuo and the residue was purified by silica gel column chromatography to afford N-[5-bromo-2-(1,3-dioxolan-2-yl)phenyl]acetamide (Intermediate 98-2) (2 g, 19%) as a yellow oil. MS (ESI) calculated for C₁₁H₁₂BrNO₃: 285.00 m/z, found 286.05, 288.05 [M+H, M+H+2]⁺.

### Intermediate 98-1: 5-bromo-2-(1,3-dioxolan-2-yl)aniline

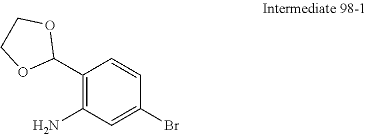 [SMILES: Nc1cc(Br)ccc1C1OCCO1]

### Synthetic Route:

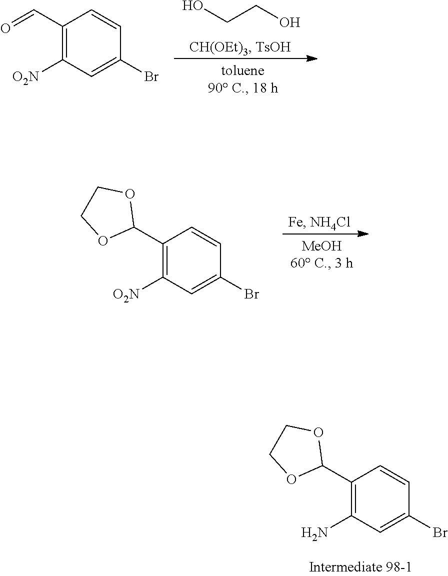

### Step 1: Synthesis of 2-(4-bromo-2-nitrophenyl)-1,3-dioxolane

To a solution of 4-bromo-2-nitrobenzaldehyde (5.00 g, 21.7 mmol, 1 equiv) in toluene (50 mL) were added ethylene glycol (6.75 g, 109 mmol, 5 equiv), triethyl orthoformate (9.66 g, 65.2 mmol, 3 equiv) and p-toluenesulfonic acid (0.37 g, 2.2 mmol, 0.1 equiv). The resulting mixture was stirred at 90° C. for 18 h under nitrogen atmosphere. The reaction was cooled to room temperature and quenched with water (30 mL), which was added dropwise over 10 min. The resulting mixture was extracted with dichloromethane (30 mL×2), and the combined extracts were washed with brine (10 mL), dried over anhydrous sodium sulfate, filtered, and concentrated in vacuo. The residue was purified by silica gel column chromatography to afford 2-(4-bromo-2-nitrophenyl)-1,3-dioxolane (1.89 g, 32%) as a yellow oil. MS (ESI) calculated for C₉HsBrNO₄: 272.96 m/z, found 273.90 [M+H]⁺.

### Step 2: Synthesis of 5-bromo-2-(1,3-dioxolan-2-yl)aniline (Intermediate 98-1)

To a solution of 2-(4-bromo-2-nitrophenyl)-1,3-dioxolane (650 mg, 2.37 mmol, 1 equiv) in methanol (10 mL) were added iron (1.32 g, 23.7 mmol, 10 equiv) and ammonium chloride (634 mg, 11.9 mmol, 5 equiv). The resulting mixture was stirred at 60° C. for 3 h. The mixture was then cooled to room temperature and filtered, rinsing with ethyl acetate. The filtrate was concentrated, and the residue was taken up into water (30 mL). The resulting mixture was extracted with ethyl acetate (3×30 mL). The organic layers were combined, dried over sodium sulfate, filtered, and concentrated in vacuo. The residue obtained was purified by silica gel chromatography using a 0-30% gradient of ethyl acetate in petroleum ether to afford 5-bromo-2-(1,3-dioxolan-2-yl)aniline (Intermediate 98-1) (300 mg, 52%) as a yellow solid. MS (ESI) calculated for C₉H₁₀BrNO₂: 242.99 m/z, found 243.98 [M+H]⁺.

### Example 99: 4-{2-[({4-[2-(2-aminopyridin-3-yl)-5-chloroimidazo[4,5-b]pyridin-3-yl]phenyl}methyl)amino]ethyl}-2-hydroxybenzaldehyde

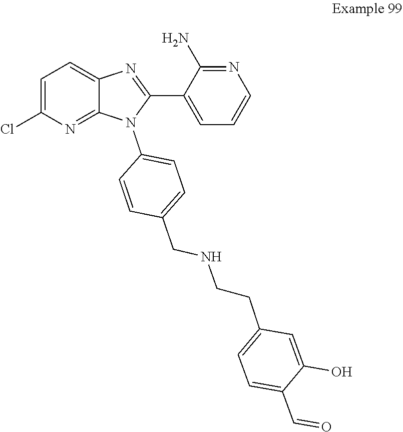 [SMILES: Nc1ncccc1-c1nc2ccc(Cl)nc2n1-c1ccc(CNCCc2ccc(C=O)c(O)c2)cc1]

Example 99 was prepared in a manner analogous to Example 19 using Intermediate 99-1 in place of Intermediate 19-2, Intermediate 16-4 in place of Intermediate 19-3 and tetrahydrofuran/triethylamine trihydrofluoride (10:1) in place of 2,2,2-trifluoroacetic acid/methanesulfonic acid. MS (ESI) calculated for C₂₇H₂₃ClN₆O₂: 498.16 m/z, found 499.15 [M+H]⁺. ¹H NMR (400 MHz, DMSO-d₆) δ (ppm): 10.19 (s, 1H), 8.32-8.35 (m, 1H), 8.04-8.05 (m, 1H), 7.52-7.69 (m, 8H), 6.90-6.92 (m, 2H), 6.67-6.68 (m, 1H), 4.28-4.29 (m, 2H), 3.25-3.27 (m, 2H), 2.97-2.98 (m, 2H).

### Intermediate 99-1: 3-(3-(4-(aminomethyl)phenyl)-5-chloro-3H-imidazo[4,5-b]pyridin-2-yl)pyridin-2-amine

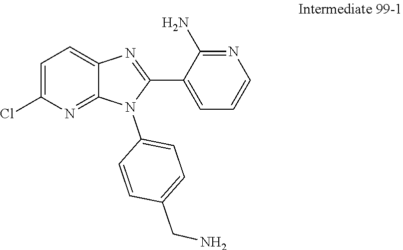 [SMILES: NCc1ccc(-n2c(-c3cccnc3N)nc3ccc(Cl)nc32)cc1]

Intermediate 99-1 was prepared in a manner analogous to Intermediate 24-2 using Intermediate 1-1 in place of Intermediate 24-1. MS (ESI) calculated for C₁₈H₁₅ClN₆: 350.10 m/z, found 351.05 [M+H]⁺.

### Example 100: N-(4-(2-(2-aminopyridin-3-yl)-5-phenyl-3H-imidazo[4,5-b]pyridin-3-yl)phenyl)-3-(4-formyl-3-hydroxyphenyl)-N-methylpropanamide

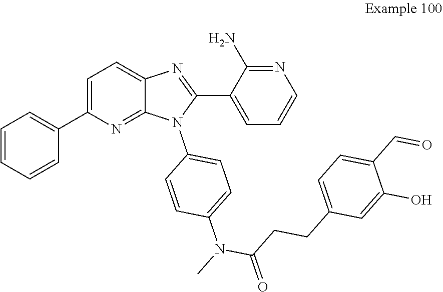 [SMILES: CN(C(=O)CCc1ccc(C=O)c(O)c1)c1ccc(-n2c(-c3cccnc3N)nc3ccc(-c4ccccc4)nc32)cc1]

Example 100 was prepared in a manner analogous to Example 1 (starting from Step 3) using Intermediate 100-1 in place of the amine starting material and Intermediate 100-2 in place of Intermediate 1-4. MS (ESI) calculated for C₃₄H₂₈N₆O₃: 568.22 m/z, found 569.20 [M+H]⁺. ¹H NMR (300 MHz, DMSO-d₆) δ (ppm): 10.16 (s, 1H), 8.27-8.29 (m, 1H), 8.01-8.03 (m, 3H), 7.91-7.98 (m, 1H), 7.42-7.58 (m, 8H), 7.10-7.13 (m, 1H), 6.71-6.77 (m, 2H), 6.10 (s, 1H), 3.20-3.26 (m, 3H), 2.75-2.89 (m, 2H), 2.36-2.52 (m, 2H).

### Intermediate 100-1: 3-(3-(4-(methylamino)phenyl)-5-phenyl-3H-imidazo[4,5-b]pyridin-2-yl)pyridin-2-amine

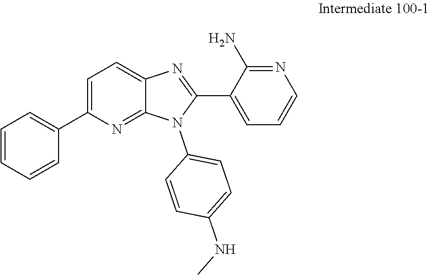 [SMILES: CNc1ccc(-n2c(-c3cccnc3N)nc3ccc(-c4ccccc4)nc32)cc1]

Intermediate 100-1 was prepared in a manner analogous to Intermediate 11-1 using tert-butyl N-(4-aminophenyl)-N-methylcarbamate in place of tert-butyl 2-(4-aminophenyl)acetate. MS (ESI) calculated for C₂₄H₂₀N₆: 392.17 m/z, found 393.25 [M+H]⁺.

### Intermediate 100-2: 3-(4-(1,3-dioxolan-2-yl)-3-((4-methoxybenzyl)oxy)phenyl)propanoic acid

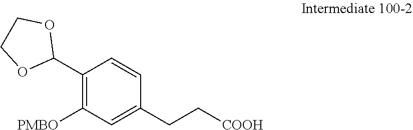

Intermediate 100-2 was prepared in a manner analogous to Intermediate 86-2 using Intermediate 1-3 in place of Intermediate 86-1. MS (ESI) calculated for C₂₀H₂₂O₆: 358.14 m/z, found 359.10 [M+H]⁺.

### Example 101: N-{3-[2-(2-aminopyridin-3-yl)-3-[4-({[2-(4-formyl-3-hydroxyphenyl) ethyl]amino}methyl)phenyl]imidazo[4,5-b]pyridin-5-yl]phenyl}methanesulfonamide

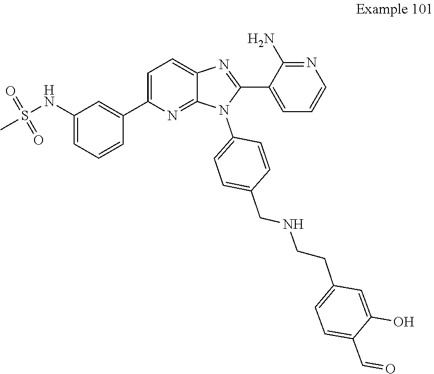 [SMILES: CS(=O)(=O)Nc1cccc(-c2ccc3nc(-c4cccnc4N)n(-c4ccc(CNCCc5ccc(C=O)c(O)c5)cc4)c3n2)c1]

Example 101 was prepared in a manner analogous to Example 19 using Intermediate 101-1 in place of Intermediate 19-2, Intermediate 16-4 in place of Intermediate 19-3 and tetrahydrofuran/triethylamine trihydrofluoride (10:1) in place of 2,2,2-trifluoroacetic acid/methanesulfonic acid. MS (ESI) calculated for C₃₄H₃₁N₇O₄S: 633.22 m/z, found 634.20 [M+H]⁺. ¹H NMR (300 MHz, DMSO-d₆) δ (ppm): 10.17 (s, 1H), 8.27 (s, 1H), 7.89-7.98 (m, 3H), 7.53-7.72 (m, 5H), 7.41 (s, 2H), 7.23 (s, 2H), 6.85-6.88 (m, 2H), 6.39 (s, 1H), 4.07-4.31 (m, 2H), 3.22-3.25 (s, 3H), 2.78-3.00 (m, 4H).

### Intermediate 101-1: N-(3-(3-(4-(aminomethyl)phenyl)-2-(2-aminopyridin-3-yl)-3H-imidazo[4,5-b]pyridin-5-yl)phenyl)methanesulfonamide

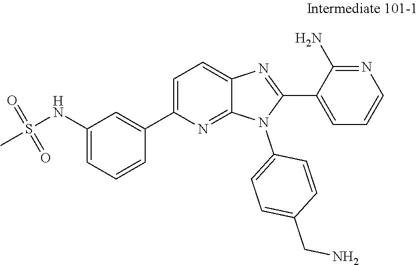 [SMILES: CS(=O)(=O)Nc1cccc(-c2ccc3nc(-c4cccnc4N)n(-c4ccc(CN)cc4)c3n2)c1]

Intermediate 101-1 was prepared in a manner analogous to Intermediate 12-1 using 3-methanesulfonamidophenylboronic acid in place of the boronic acid. MS (ESI) calculated for C₂₅H₂₃N₇O₂S: 485.16 m/z, found 486.15 [M+H]⁺.

### Example 102: N-{3-[2-(2-aminopyridin-3-yl)-3-[4-({[2-(2-fluoro-4-formyl-3-hydroxy phenyl)ethyl]amino}methyl)phenyl]imidazo[4,5-b]pyridin-5-yl]phenyl}acetamide

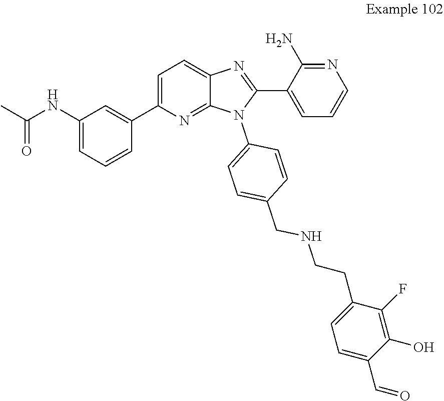 [SMILES: CC(=O)Nc1cccc(-c2ccc3nc(-c4cccnc4N)n(-c4ccc(CNCCc5ccc(C=O)c(O)c5F)cc4)c3n2)c1]

Example 102 was prepared in a manner analogous to Example 19 using Intermediate 102-1 in place of Intermediate 19-2 and Intermediate 88-1 in place of Intermediate 19-3. MS (ESI) calculated for C₃₅H₃₀FN₇O₃: 615.24 m/z, found 616.25 [M+H]⁺. ¹H NMR (400 MHz, DMSO-d₆) δ (ppm): 10.17 (s, 1H), 8.28-8.31 (m, 2H), 8.01-8.22 (m, 1H), 7.94-7.99 (m, 1H), 6.93-7.03 (m, 2H), 7.65-7.71 (m, 6H), 7.59-7.62 (m, 2H), 7.41-7.56 (m, 1H), 6.45-6.46 (m, 1H), 6.41-6.43 (m, 1H), 4.18 (s, 2H), 3.10-3.11 (m, 2H), 2.98 -2.99 (m, 2H), 2.05-2.08 (m, 3H). ¹⁹F NMR (400 MHz, DMSO-d₆) δ (ppm): −140.71.

### Intermediate 102-1: N-(3-(3-(4-(aminomethyl)phenyl)-2-(2-aminopyridin-3-yl)-3H-imidazo[4,5-b]pyridin-5-yl)phenyl)acetamide

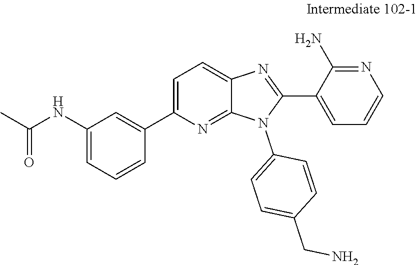 [SMILES: CC(=O)Nc1cccc(-c2ccc3nc(-c4cccnc4N)n(-c4ccc(CN)cc4)c3n2)c1]

Intermediate 102-1 was prepared in a manner analogous to Intermediate 12-1 using 3-acetamidophenylboronic acid in place of the boronic acid. MS (ESI) calculated for C₂₆H₂₃N₇O: 449.20 m/z, found 450.20 [M+H]⁺.

### Example 103: 4-{2-[({4-[2-(2-aminopyridin-3-yl)-5-(pyridin-2-yl)imidazo[4,5-b]pyridin-3-yl]phenyl}methyl)amino]ethyl}-2-hydroxybenzaldehyde

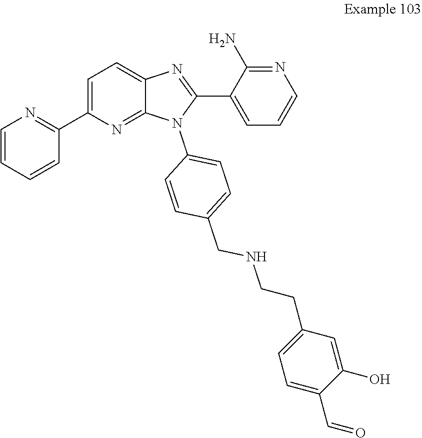 [SMILES: Nc1ncccc1-c1nc2ccc(-c3ccccn3)nc2n1-c1ccc(CNCCc2ccc(C=O)c(O)c2)cc1]

Example 103 was prepared in a manner analogous to Example 16 using Intermediate 103-1 in place of Intermediate 16-2. MS (ESI) calculated for C₃₂H₂₇N₇O₂: 541.22 m/z, found 542.25 [M+H]⁺. ¹H NMR (400 MHz, DMSO-d₆) δ (ppm): 10.10-10.21 (m, 1H), 8.67-8.74 (m, 1H), 8.43-8.54 (m, 1H), 8.34-8.43 (m, 1H), 8.18-8.24 (m, 1H), 8.00-8.09 (m, 1H), 7.86-7.96 (m, 1H), 7.63-7.75 (m, 5H), 7.54-7.60 (m, 1H), 7.44-7.52 (m, 1H), 6.90-6.99 (m, 2H), 6.66-6.70 (m, 1H), 4.28-4.36 (m, 2H), 3.23-3.33 (m, 2H), 2.98-3.06 (m, 2H).

### Intermediate 103-1: 3-(3-(4-(aminomethyl)phenyl)-5-(pyridin-2-yl)-3H-imidazo[4,5-b]pyridin-2-yl)pyridin-2-amine

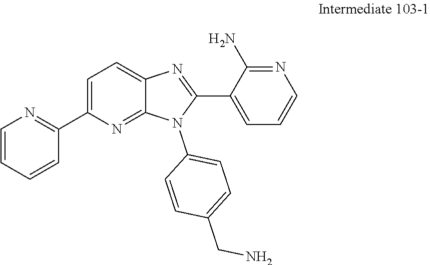 [SMILES: NCc1ccc(-n2c(-c3cccnc3N)nc3ccc(-c4ccccn4)nc32)cc1]

### Synthetic Route:

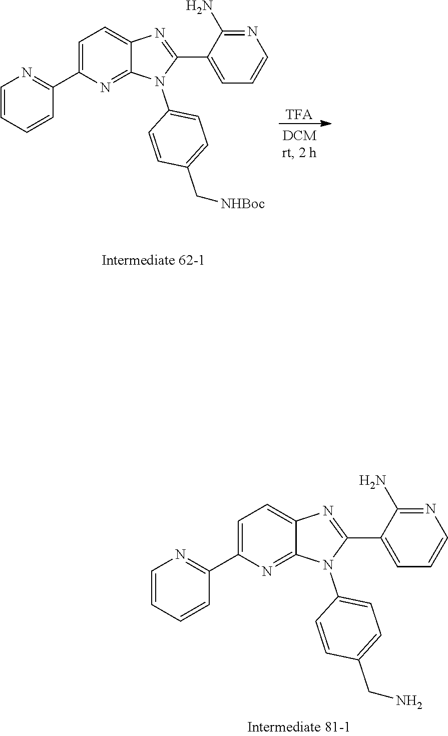 [SMILES: CC(C)(C)OC(=O)NCc1ccc(-n2c(-c3cccnc3N)nc3ccc(-c4ccccn4)nc32)cc1 ; NCc1ccc(-n2c(-c3cccnc3N)nc3ccc(-c4ccccn4)nc32)cc1 ; "TFA"]

Step 1: Synthesis of 3-(3-(4-(aminomethyl)phenyl)-5-(pyridin-2-yl)-3H-imidazo[4,5-b]pyridin-2-yl)pyridin-2-amine (Intermediate 103-1)

tert-Butyl (4-(2-(2-aminopyridin-3-yl)-5-(pyridin-2-yl)-3H-imidazo[4,5-b]pyridin-3-yl)benzyl)carbamate (Intermediate 62-1) (150 mg, 0.304 mmol, 1 equiv) was dissolved in dichloromethane (5 mL) and 2,2,2-trifluoroacetic acid (1 mL) was added. The resulting mixture was stirred at room temperature for 2 h. The mixture was concentrated in vacuo and the residue was purified by reverse-phase flash column chromatography using a 6-60% gradient of acetonitrile in water (+0.05% ammonium bicarbonate) to afford 3-{3-[4-(aminomethyl)phenyl]-5-(pyridin-2-yl)imidazo[4,5-b]pyridin-2-yl}pyridin-2-amine (Intermediate 103-1) (100 mg, 84%) as a yellow solid. MS (ESI) calculated for C₂₃H₁₉N₇: 393.17 m/z, found 394.15 [M+H]⁺.

### Example 104: 4-(2-(2-aminopyridin-3-yl)-5-phenyl-3H-imidazo[4,5-b]pyridin-3-yl)-N-(3-(4-formyl-3-hydroxyphenyl)prop-2-yn-1-yl)benzamide

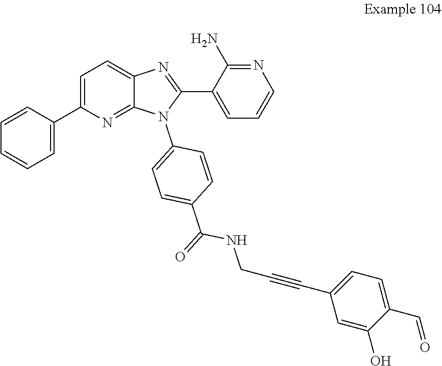 [SMILES: Nc1ncccc1-c1nc2ccc(-c3ccccc3)nc2n1-c1ccc(C(=O)NCC#Cc2ccc(C=O)c(O)c2)cc1]

### Synthetic Route:

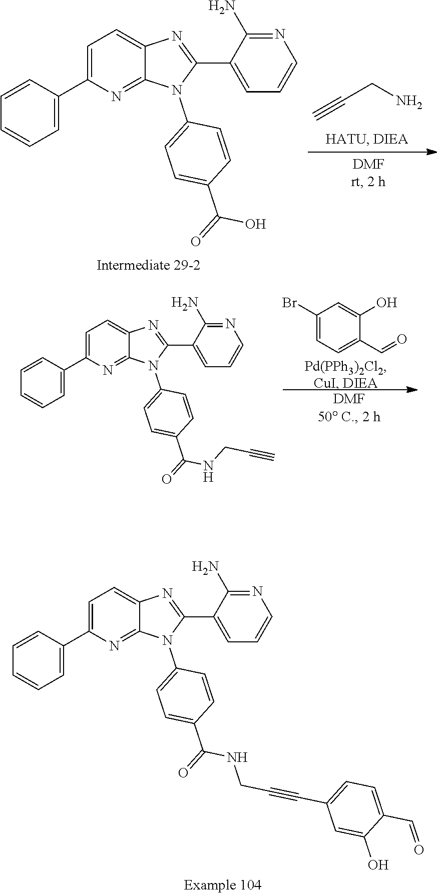

### Step 1: Synthesis of 4-(2-(2-aminopyridin-3-yl)-5-phenyl-3H-imidazo[4,5-b]pyridin-3-yl)-N-(prop-2-yn-1-yl)benzamide

To a solution of 4-(2-(2-aminopyridin-3-yl)-5-phenyl-3H-imidazo[4,5-b]pyridin-3-yl)benzoic acid (Intermediate 29-2) (1.0 g, 2.5 mmol, 1 equiv) in N,N-dimethylformamide (30 mL) were added 2-propynylamine (0.20 g, 3.7 mmol, 1.5 equiv), N,N-diisopropylethylamine (0.95 g, 7.4 mmol, 3.0 equiv), and HATU (1.03 g, 2.70 mmol, 1.1 equiv). The resulting solution was stirred at room temperature for 2 h. The reaction was quenched by the addition of saturated aqueous ammonium chloride (20 mL) at 0° C. The aqueous layer was extracted with ethyl acetate (3×20 mL). The combined organic layers were concentrated under reduced pressure and the residue was purified by silica gel column chromatography eluting with methanol/dichloromethane (1:20) to afford 4-(2-(2-aminopyridin-3-yl)-5-phenyl-3H-imidazo[4,5-b]pyridin-3-yl)-N-(prop-2-yn-1-yl)benzamide (500 mg, 45.83%) as a yellow solid. MS (ESI) calculated for C₂₇H₂₀N₆O: 444.17 m/z, found 445.20 [M+H]⁺.

### Step 2: Synthesis of 4-(2-(2-aminopyridin-3-yl)-5-phenyl-3H-imidazo[4,5-b]pyridin-3-yl)-N-(3-(4-formyl-3-hydroxyphenyl)prop-2-yn-1-yl)benzamide (Example 104)

A mixture of 4-[2-(2-aminopyridin-3-yl)-5-phenylimidazo[4,5-b]pyridin-3-yl]-N-(prop-2-yn-1-yl)benzamide (250 mg, 0.562 mmol, 1 equiv), 4-bromo-2-hydroxybenzaldehyde (226 mg, 1.12 mmol, 2.0 equiv), bis(triphenylphosphine)palladium(II) dichloride (39 mg, 0.056 mmol, 0.1 equiv), and copper (I) iodide (11 mg, 0.056 mmol, 0.1 equiv) was dissolved in degassed N,N-diisopropylethylamine (218 mg, 1.69 mmol, 3 equiv) and N,N-dimethylformamide (5 mL) under nitrogen atmosphere. The resulting solution was stirred at 50° C. for 2 h. The reaction was quenched with saturated aqueous ammonium chloride (10 mL). The mixture was extracted with ethyl acetate (3×20 mL) and the combined organic layers were concentrated under reduced pressure. The crude product was purified by Preparative HPLC on a XBridge Shield RP18 OBD Column using a 52-73% gradient of acetonitrile in water (+0.1% formic acid) to afford 4-(2-(2-aminopyridin-3-yl)-5-phenyl-3H-imidazo[4,5-b]pyridin-3-yl)-N-(3-(4-formyl-3-hydroxyphenyl)prop-2-yn-1-yl)benzamide (Example 104) (45 mg, 14%) as a yellow solid. MS (ESI) calculated for C₃₄H₂₄N₆O₃: 564.19 m/z, found 565.15 [M+H]⁺. ¹H NMR (400 MHz, DMSO-d₆) δ (ppm): 11.32 (br s, 1H), 10.24 (s, 1H), 10.07 (s, 1H), 9.10 (s, 1H), 8.93-8.95 (m, 1H), 8.76-8.78 (m, 1H), 8.36-8.38 (m, 1H), 8.22-8.24 (m, 1H), 8.17-8.19 (m, 2H), 7.92-7.94 (m, 2H), 7.84-7.86 (m, 2H), 7.73-7.75 (m, 1H), 7.55-7.61 (m, 3H), 7.48-7.52 (m, 1H), 7.24-7.28 (m, 1H), 7.00 (s, 1H), 6.92-6.94 (m, 1H), 4.34-4.35 (m, 2H).

### Example 105: (R)-5-(2-((1-(4-(2-(2-aminopyridin-3-yl)-5-phenyl-3H-imidazo[4,5-b]pyridin-3-yl)phenyl)ethyl)amino)ethyl)-2-hydroxybenzaldehyde

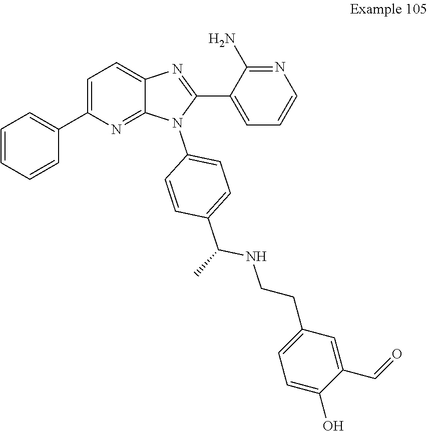 [SMILES: C[C@@H](NCCc1ccc(O)c(C=O)c1)c1ccc(-n2c(-c3cccnc3N)nc3ccc(-c4ccccc4)nc32)cc1]

Example 105 was prepared in a manner analogous to Example 92 (via Intermediates 92-2 and 92-1) starting from of (R)-1-(4-nitrophenyl)ethan-1-amine in place of (S)-1-(4-nitrophenyl)ethan-1-amine. MS (ESI) calculated for C₃₄H₃₀N₆O₂: 554.25 m/z, found 555.15 [M+H]⁺. ¹H NMR (300 MHz, DMSO-d₆) δ (ppm): 10.23 (s, 1H), 9.10 (s, 1H), 8.78-8.79 (m, 1H), 8.38-8.42 (m, 1H), 8.20-8.23 (m, 1H), 8.13-8.15 (m, 2H), 7.84-7.87 (m, 2H), 7.68-7.70 (m, 1H), 7.49-7.54 (m, 6H), 7.36-7.39 (m, 1H), 7.27-7.31 (m, 1H), 6.94-6.97 (m, 1H), 4.36-4.41 (m, 1H), 3.06-3.09 (m, 1H), 2.87-2.90 (m, 3H), 1.58-1.60 (m, 3H).

### Example 106: 4-[2-(2-aminopyridin-3-yl)-5-phenylimidazo[4,5-b]pyridin-3-yl]-N-[2-(4-formyl-3-hydroxyphenyl)ethyl]benzenesulfonamide

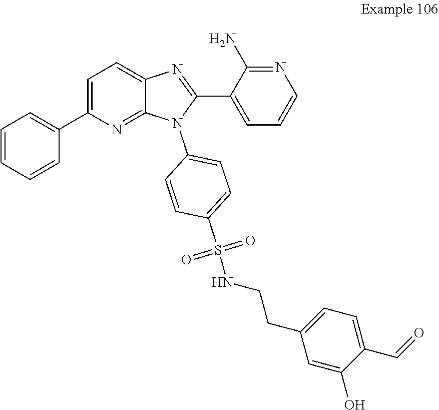 [SMILES: Nc1ncccc1-c1nc2ccc(-c3ccccc3)nc2n1-c1ccc(S(=O)(=O)NCCc2ccc(C=O)c(O)c2)cc1]

### Synthetic Route:

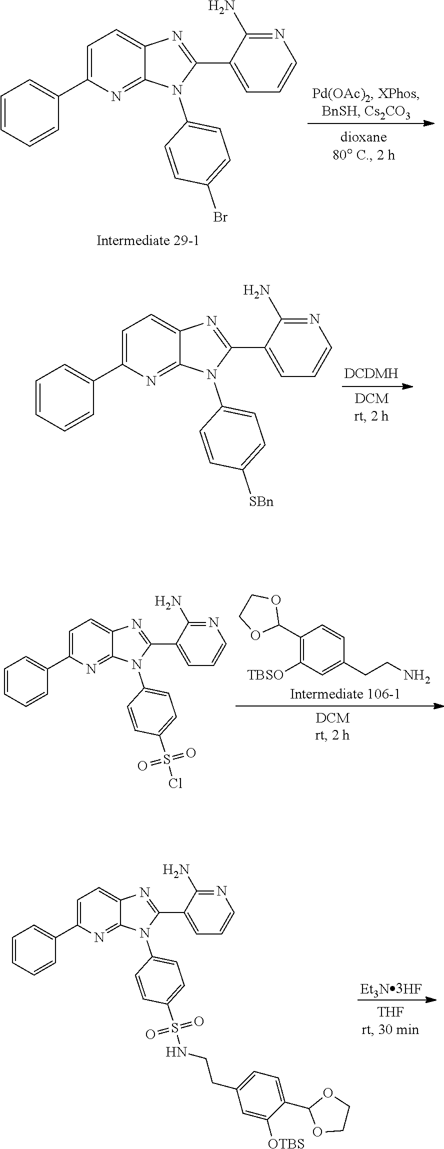

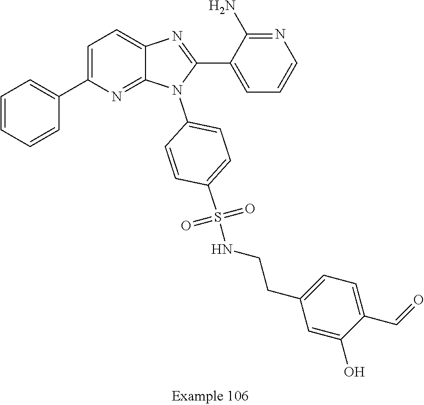 [SMILES: Nc1ncccc1-c1nc2ccc(-c3ccccc3)nc2n1-c1ccc(S(=O)(=O)NCCc2ccc(C=O)c(O)c2)cc1]

### Step 1: Synthesis of 3-(3-(4-(benzylthio)phenyl)-5-phenyl-3H-imidazo[4,5-b]pyridin-2-yl)pyridin-2-amine

To a solution of 3-(3-(4-bromophenyl)-5-phenyl-3H-imidazo[4,5-b]pyridin-2-yl)pyridin-2-amine (Intermediate 29-1) (0.44 g, 1.0 mmol, 1 equiv) in 1,4-dioxane (10 mL) were added palladium(II) acetate (17 mg, 0.10 mmol, 0.1 equiv), benzyl thiol (541 mg, 5.0 mmol, 5.0 equiv) and XPhos (95 mg, 0.20 mmol, 0.2 equiv). After stirring for 2 h at 80° C. under nitrogen atmosphere, the reaction was cooled to 0° C. and quenched by the addition of water (10 mL). The resulting mixture was extracted with ethyl acetate (20 L×3). The combined organic layers were washed with water (10 mL×3), dried over anhydrous sodium sulfate, filtered, and concentrated under reduced pressure. The resulting residue was purified by reverse-phase flash column chromatography on C18 silica gel using a 20-95% gradient of acetonitrile in water (+0.05% ammonium bicarbonate) to afford 3-(3-(4-(benzylthio)phenyl)-5-phenyl-3H-imidazo[4,5-b]pyridin-2-yl)pyridin-2-amine (300 mg, 62%). MS (ESI) calculated for C₃₀H₂₃N₅S: 485.17 m/z, found 486.10 [M+H]⁺.

### Step 2: Synthesis of 4-[2-(2-aminopyridin-3-yl)-5-phenylimidazo[4,5-b]pyridin-3-yl]benzenesulfonyl chloride

To a solution of 3-{3-[4-(benzylsulfanyl)phenyl]-5-phenylimidazo[4,5-b]pyridin-2-yl}pyridin-2-amine (200 mg, 0.412 mmol, 1 equiv) in dichloromethane (1.5 mL), acetic acid (0.2 mL) and water (0.1 mL) was added 1,3-dichloro-5,5-dimethylimidazolidine-2,4-dione (243 mg, 1.24 mmol, 3 equiv) and the resulting mixture was stirred at room temperature for 1 h. The reaction mixture was concentrated in vacuo and the residue was purified by silica gel column chromatography using a 0-10% gradient of methanol in dichloromethane to provide 4-[2-(2-aminopyridin-3-yl)-5-phenylimidazo[4,5-b]pyridin-3-yl]benzenesulfonyl chloride (160 mg, 84%) as a yellow solid. MS (ESI) calculated for C₂₃H₁₆ClN₅O₂S: 461.07 m/z, found 461.95 [M+H]⁺.

### Step 3: Synthesis of 4-[2-(2-aminopyridin-3-yl)-5-phenylimidazo[4,5-b]pyridin-3-yl]-N-(2-{3-[(tert-butyldimethylsilyl)oxy]-4-(1,3-dioxolan-2-yl)phenyl}ethyl)benzenesulfonamide

To a solution of 4-[2-(2-aminopyridin-3-yl)-5-phenylimidazo[4,5-b]pyridin-3-yl]benzenesulfonyl chloride (150 mg, 0.325 mmol, 1 equiv) in dichloromethane (3 mL) was added 2-{4-[(tert-butyldimethylsilyl)oxy]-3-(1,3-dioxolan-2-yl)phenyl}ethanamine (Intermediate 106-1) (105 mg, 0.325 mmol, 1 equiv). The resulting mixture was stirred at room temperature for 2 h. After concentration, the residue was purified by reverse-phase flash column chromatography using a 10-95% gradient of acetonitrile in water (+0.05% 2,2,2-trifluoroacetic acid) to afford 4-[2-(2-aminopyridin-3-yl)-5-phenylimidazo[4,5-b]pyridin-3-yl]-N-(2-{3-[(tert-butyldimethylsilyl) oxy]-4-(1,3-dioxolan-2-yl)phenyl}ethyl)benzenesulfonamide (50 mg, 21%) as a yellow solid. MS (ESI) calculated for C₄₀H₄₄N₆O₅SSi: 748.29 m/z, found 747.10 [M−H]⁻.

### Step 4: Synthesis of 4-[2-(2-aminopyridin-3-yl)-5-phenylimidazo[4,5-b]pyridin-3-yl]-N-[2-(4-formyl-3-hydroxyphenyl)ethyl]benzenesulfonamide (Example 106)

To a solution of 4-[2-(2-aminopyridin-3-yl)-5-phenylimidazo[4,5-b]pyridin-3-yl]-N-(2-{3-[(tert-butyldimethylsilyl)oxy]-4-(1,3-dioxolan-2-yl)phenyl}ethyl)benzenesulfonamide (50 mg, 0.067 mmol, 1 equiv) in 1,4-dioxane (5 mL) was added triethylamine trihydrofluoride (108 mg, 0.670 mmol, 10 equiv). The resulting mixture was stirred at room temperature for 30 min. The mixture was then concentrated in vacuo and purified by preparative HPLC on a XBridge Prep Phenyl OBD Column using a 28-48% gradient of acetonitrile in water to afford 4-[2-(2-aminopyridin-3-yl)-5-phenylimidazo[4,5-b]pyridin-3-yl]-N-[2-(4-formyl-3-hydroxyphenyl) ethyl]benzenesulfonamide (Example 106) (6.9 mg, 17%) as a light-yellow solid. MS (ESI) calculated for C₃₂H₂₆N₆O₄S: 590.17 m/z, found 591.15 [M+H]⁺. ¹H NMR (300 MHz, DMSO-d₆) δ (ppm): 9.85 (s, 1H), 8.32-8.41 (m, 1H), 8.04-8.15 (m, 6H), 7.81-7.92 (m, 3H), 7.64-7.73 (m, 1H), 7.40-7.60 (m, 5H), 6.78-6.89 (m, 1H), 2.95-3.17 (m, 4H).

### Intermediate 106-1: 2-(3-((tert-butyldimethylsilyl)oxy)-4-(1,3-dioxolan-2-yl)phenyl)ethan-1-amine

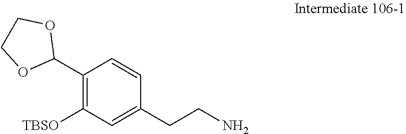 [SMILES: CC(C)(C)[Si](C)(C)Oc1cc(CCN)ccc1C1OCCO1]

### Synthetic Route:

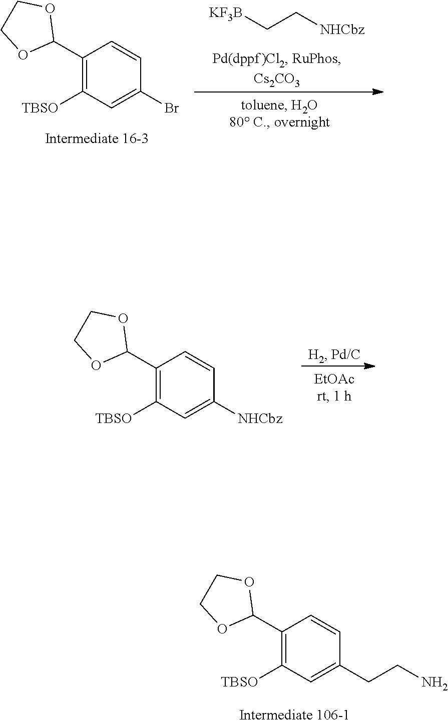

### Step 1: Synthesis of benzyl (3-((tert-butyldimethylsilyl)oxy)-4-(1,3-dioxolan-2-yl)phenethyl)carbamate

A mixture of 5-bromo-2-(1,3-dioxolan-2-yl)phenoxy(tert-butyl)dimethylsilane (Intermediate 16-3) (1.00 g, 2.78 mmol, 1 equiv), benzyl N-[2-(trifluoro-lambda4-boranyl)ethyl]carbamate potassium (952 mg, 3.34 mmol, 1.2 equiv), cesium carbonate (1.81 mg, 5.57 mmol, 2 equiv), [1,1′-bis(diphenylphosphino)ferrocene]dichloropalladium(II) (204 mg, 0.278 mmol, 0.1 equiv) and RuPhos (130 mg, 0.278 mmol, 0.1 equiv) in toluene (10 mL) and water (1 mL) was stirred overnight at 80° C. under nitrogen atmosphere. The mixture was cooled to room temperature, diluted with ethyl acetate (100 mL), washed with water (50 mL×3), dried over anhydrous sodium sulfate, filtered, and concentrated under reduced pressure. The resulting residue was purified by silica gel column chromatography using a 0-100% gradient of ethyl acetate in petroleum ether to afford benzyl (3-((tert-butyldimethylsilyl)oxy)-4-(1,3-dioxolan-2-yl)phenethyl)carbamate (800 mg, 63%) as a colorless oil. MS (ESI) calculated for C₂₅H₃₅NO₅Si: 457.23 m/z, found 458.25 [M+H]⁺.

### Step 2: Synthesis of 2-(3-((tert-butyldimethylsilyl)oxy)-4-(1,3-dioxolan-2-yl)phenyl)ethan-1-amine (Intermediate 106-1)

To a solution of benzyl N-(2-{3-[(tert-butyldimethylsilyl)oxy]-4-(1,3-dioxolan-2-yl)phenyl}ethyl)carbamate (800 mg, 1.75 mmol, 1 equiv) in methanol (40 mL) and ethyl acetate (40 mL) was added 10% palladium on carbon (400 mg, 0.20 equiv) under nitrogen atmosphere. The mixture was stirred for 1 h at room temperature under hydrogen atmosphere. The resulting mixture was filtered through celite, and the filtrate was concentrated in vacuo to afford 2-(3-((tert-butyldimethylsilyl)oxy)-4-(1,3-dioxolan-2-yl)phenyl)ethan-1-amine (Intermediate 106-1) (1.2 g, 85%) as a yellow oil. MS (ESI) calculated for C₁₇H₂₉NO₃Si: 323.19 m/z, found 324.20 [M+H]⁺.

### Example 107: 4-(2-((4-(2-(2-aminopyridin-3-yl)-5-morpholino-3H-imidazo[4,5-b]pyridin-3-yl)benzyl)amino)ethyl)-2-hydroxybenzaldehyde

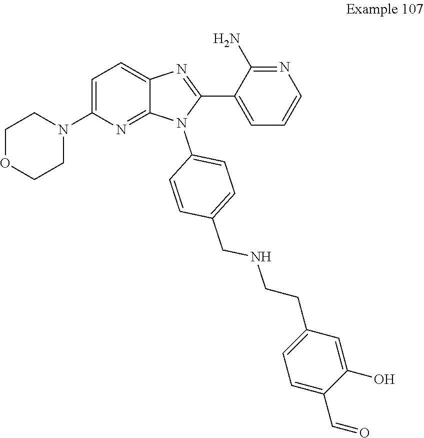 [SMILES: Nc1ncccc1-c1nc2ccc(N3CCOCC3)nc2n1-c1ccc(CNCCc2ccc(C=O)c(O)c2)cc1]

Example 107 was prepared in a manner analogous to Example 19 using Intermediate 3-1 in place of Intermediate 19-2 and Intermediate 16-4 in place of Intermediate 19-3. MS (ESI) calculated for C₃₁H₃₁N₇O₃: 549.25 m/z, found 550.15 [M+H]⁺. ¹H-NMR (300 MHz, DMSO-d₆) δ (ppm): 10.20 (s, 1H), 7.95-8.03 (m, 2H), 7.60-7.69 (m, 3H), 7.46-7.49 (m, 2H), 7.12-7.14 (m, 1H), 6.89-6.94 (m, 3H), 6.36-6.40 (m, 1H), 4.26-4.27 (m, 2H), 3.61-3.68 (m, 4H), 3.31-3.41 (m, 4H), 3.20-3.25 (m, 2H), 2.94-2.99 (m, 2H).

### Example 108: 4-(2-((4-(2-(2-aminopyridin-3-yl)-5-(3-morpholinophenyl)-3H-imidazo[4,5-b]pyridin-3-yl)benzyl)amino)ethyl)-2-hydroxybenzaldehyde

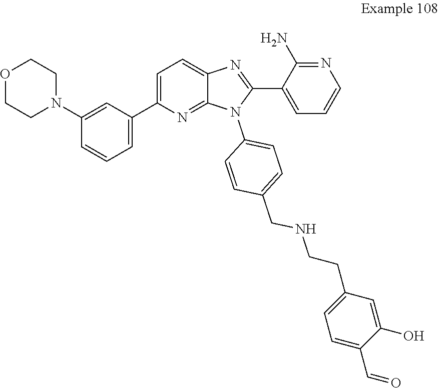 [SMILES: Nc1ncccc1-c1nc2ccc(-c3cccc(N4CCOCC4)c3)nc2n1-c1ccc(CNCCc2ccc(C=O)c(O)c2)cc1]

Example 108 was prepared in a manner analogous to Example 16 using Intermediate 12-1 in place of Intermediate 16-2. MS (ESI) calculated for C₃₇H₃₅N₇O₃: 625.28 m/z, found 626.30 [M+H]⁺. ¹H NMR (300 MHz, DMSO-d₆) δ (ppm): 10.19 (s, 1H), 8.30-8.33 (m, 1H), 8.03-8.07 (m, 2H), 7.58-7.69 (m, 7H), 7.46-7.49 (m, 1H), 7.30-7.35 (m, 1H), 6.90-7.03 (m, 3H), 6.64-6.69 (m, 1H), 4.30 (s, 2H), 3.73-3.79 (m, 4H), 3.26-3.28 (m, 2H), 3.16-3.23 (m, 4H), 2.96-3.01 (m, 2H).

### Example 109: N-(1-{3-[2-(2-aminopyridin-3-yl)-3-[4-({[2-(4-formyl-3-hydroxyphenyl) ethyl]amino}methyl)phenyl]imidazo[4,5-b]pyridin-5-yl]phenyl}piperidin-4-yl)-N-methylacetamide

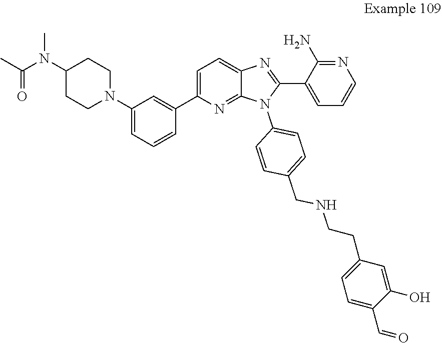 [SMILES: CC(=O)N(C)C1CCN(c2cccc(-c3ccc4nc(-c5cccnc5N)n(-c5ccc(CNCCc6ccc(C=O)c(O)c6)cc5)c4n3)c2)CC1]

Example 109 was prepared in a manner analogous to Example 42 (via Intermediate 42-1) starting from N-methyl-N-(piperidin-4-yl)acetamide in place of 1-(piperidin-4-yl)pyrrolidin-2-one and using 1,3-dibromobenzene in place of 1,3-dibromo-2-fluorobenzene. MS (ESI) calculated for C₄₁H₄₂N₈O₃: 694.34 m/z, found 695.25 [M+H]⁺. ¹H NMR (300 MHz, DMSO-d₆) δ (ppm): 10.21 (s, 1H), 8.27-8.30 (s, 1H), 8.00-8.04 (m, 2H), 7.60-7.69 (m, 6H), 7.26-7.45 (m, 3H), 7.01-7.12 (m, 1H), 6.88-6.90 (m, 2H), 6.52-6.54 (m, 1H), 4.42-4.44 (m, 1H), 4.30 (s, 2H), 3.83-3.87 (m, 2H), 3.21-3.26 (m, 2H), 2.95-3.00 (m, 2H), 2.82 (s, 3H), 2.74 (s, 2H), 2.09 (s, 1H), 2.00 (s, 2H), 1.75-1.87 (m, 3H), 1.56-1.60 (m, 1H).

### Example 110: N-(5-(2-((4-(2-(2-aminopyridin-3-yl)-5-phenyl-3H-imidazo[4,5-b]pyridin-3-yl)benzyl)amino)ethyl)-2-formylphenyl)acetamide

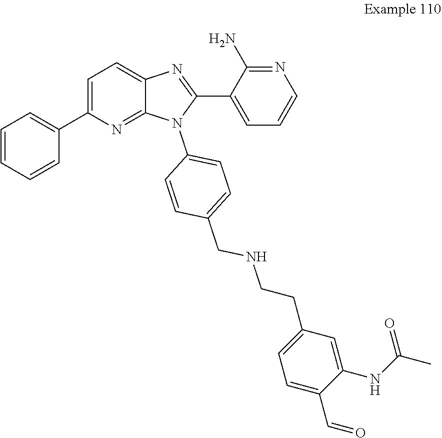 [SMILES: CC(=O)Nc1cc(CCNCc2ccc(-n3c(-c4cccnc4N)nc4ccc(-c5ccccc5)nc43)cc2)ccc1C=O]

Example 110 was prepared in a manner analogous to Example 19 using Intermediate 4-1 in place of Intermediate 19-3 and Intermediate 98-3 in place of Intermediate 19-3. MS (ESI) calculated for C₃₅H₃₁N₇O₂: 581.25, found 582.15 [M+H]⁺. ¹H-NMR (300 MHz, DMSO-d₆) δ (ppm): 9.92 (s, 1H), 8.22-8.29 (m, 1H), 8.09 (s, 1H), 7.98-8.03 (m, 4H), 7.78-7.80 (m, 1H), 7.37-7.54 (m, 8H), 7.18-7.20 (m, 2H), 6.34-6.38 (m, 1H), 3.89 (s, 2H), 2.82-2.86 (m, 4H), 2.10-2.14 (m, 3H).

### Example 111: N-(2-{4-[2-(2-aminopyridin-3-yl)-5-phenylimidazo[4,5-b]pyridin-3-yl]phenyl}ethyl)-4-formyl-3-hydroxybenzamide

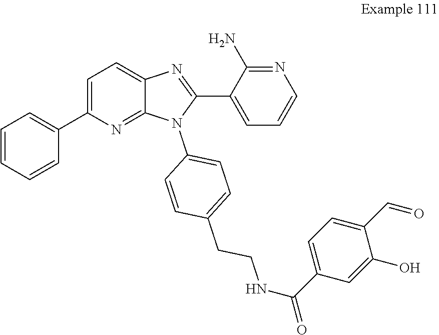 [SMILES: Nc1ncccc1-c1nc2ccc(-c3ccccc3)nc2n1-c1ccc(CCNC(=O)c2ccc(C=O)c(O)c2)cc1]

### Synthetic Route:

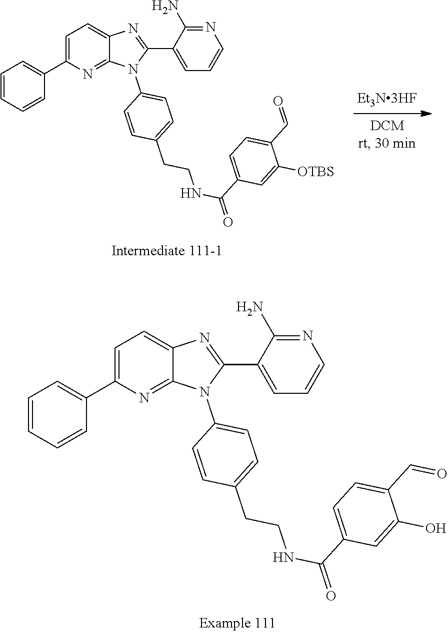 [SMILES: CC(C)(C)[Si](C)(C)Oc1cc(C(=O)NCCc2ccc(-n3c(-c4cccnc4N)nc4ccc(-c5ccccc5)nc43)cc2)ccc1C=O ; Nc1ncccc1-c1nc2ccc(-c3ccccc3)nc2n1-c1ccc(CCNC(=O)c2ccc(C=O)c(O)c2)cc1]

### Step 1: Synthesis of N-(2-{4-[2-(2-aminopyridin-3-yl)-5-phenylimidazo[4,5-b]pyridin-3-yl]phenyl}ethyl)-4-formyl-3-hydroxybenzamide (Example 111)

Triethylamine trihydrofluoride (45 mg, 0.28 mmol) was added to a solution of N-(2-{4-[2-(2-aminopyridin-3-yl)-5-phenylimidazo[4,5-b]pyridin-3-yl]phenyl}ethyl)-3-[(tert-butyldimethylsilyl)oxy]-4-(1,3-dioxolan-2-yl)benzamide (Intermediate 111-2) (90 mg, 0.14 mmol) in tetrahydrofuran (1 mL) and the mixture was stirred at room temperature for 30 min. The solvent was evaporated under reduced pressure and the crude product was purified by preparative HPLC on a XSelect CSH C18 OBD Column using a 27-47% gradient of acetonitrile in water to afford N-(2-{4-[2-(2-aminopyridin-3-yl)-5-phenylimidazo[4,5-b]pyridin-3-yl]phenyl}ethyl)-4-formyl-3-hydroxybenzamide (Example 111) (26 mg, 33%) as a yellow solid. MS (ESI) calculated for C₃₃H₂₆N₆O₃: 554.21 m/z, found 555.15 [M+H]⁺. ¹HNMR (400 MHz, DMSO-d₆) δ (ppm): 10.30 (s, 1H), 8.31-8.33 (m, 1H), 8.00-8.04 (m, 4H), 7.71-7.73 (m, 1H), 7.57-7.59 (m, 1H), 7.33-7.49 (m, 8H), 7.31-7.32 (m, 1H), 6.65-6.68 (m, 1H), 3.55-3.60 (m, 2H), 2.95-2.98 (m, 2H).

### Intermediate 111-1: N-(4-(2-(2-aminopyridin-3-yl)-5-phenyl-3H-imidazo[4,5-b]pyridin-3-yl)phenethyl)-3-((tert-butyldimethylsilyl)oxy)-4-formylbenzamide

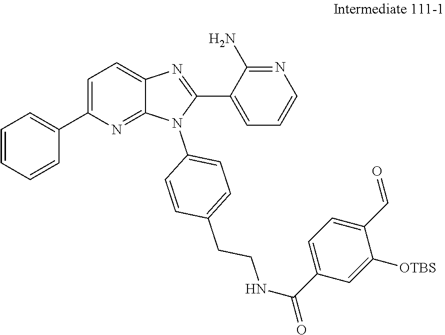 [SMILES: CC(C)(C)[Si](C)(C)Oc1cc(C(=O)NCCc2ccc(-n3c(-c4cccnc4N)nc4ccc(-c5ccccc5)nc43)cc2)ccc1C=O]

Intermediate 111-1 was prepared in a manner analogous to Example 1 (starting from Step 3) using Intermediate 36-1 in place of the amine starting material and Intermediate 111-2 in place of Intermediate 1-4. MS (ESI) calculated for C₃₉H₄₀N₆O₃Si: 668.29 m/z, found 669.00 [M+H]⁺.

### Intermediate 111-2: 3-((tert-butyldimethylsilyl)oxy)-4-(1,3-dioxolan-2-yl)benzoic acid

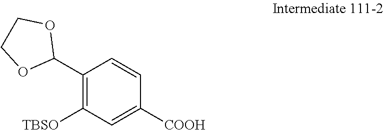 [SMILES: CC(C)(C)[Si](C)(C)Oc1cc(C(=O)O)ccc1C1OCCO1]

Intermediate 111-2 was prepared in a manner analogous to Intermediate 3-4 using Intermediate 16-3 in place of Intermediate 3-3. MS (ESI) calculated for C₁₈H₂₁ClN₄O₄: 392.13 m/z, found 393.15 [M+H]⁺.

### Example 112: 6-[2-(2-aminopyridin-3-yl)-5-phenylimidazo[4,5-b]pyridin-3-yl]-N-[2-(4-formyl-3-hydroxyphenyl)ethyl]pyridine-3-carboxamide

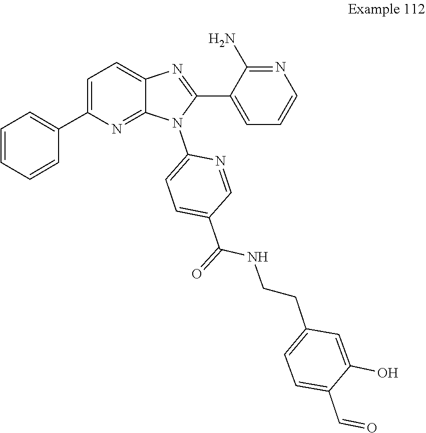 [SMILES: Nc1ncccc1-c1nc2ccc(-c3ccccc3)nc2n1-c1ccc(C(=O)NCCc2ccc(C=O)c(O)c2)cn1]

Example 112 was prepared in a manner analogous to Example 1 (starting from Step 3) using Intermediate 106-1 in place of the amine starting material, Intermediate 112-1 in place of Intermediate 1-4, HATU in place of PyBOP and tetrahydrofuran/triethylamine trihydrofluoride (10:1) in place of 2,2,2-trifluoroacetic acid/methanesulfonic acid. MS (ESI) calculated for C₃₂H₂₅N₇O₃: 555.20 m/z, found 556.20 [M+H]⁺. ¹H NMR (400 MHz, DMSO-d₆) δ (ppm): 10.10 (s, 1H), 8.71-8.76 (m, 1H), 8.47-8.60 (m, 3H), 8.17-8.25 (m, 2H), 8.11-8.17 (m, 2H), 7.93-8.00 (m, 1H), 7.63-7.69 (m, 1H), 7.54-7.62 (m, 2H), 7.46-7.53 (m, 1H), 7.23-7.30 (m, 1H), 6.94-7.00 (m, 1H), 6.89-6.93 (m, 1H), 3.51-3.61 (m, 2H), 2.87-2.95 (m, 2H).

### Intermediate 112-1: 6-(2-(2-aminopyridin-3-yl)-5-phenyl-3H-imidazo[4,5-b]pyridin-3-yl)nicotinic acid

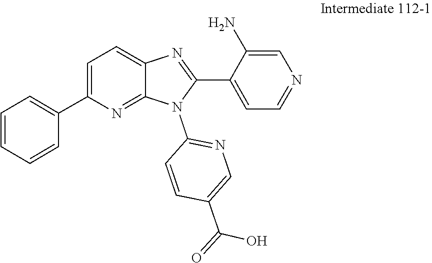 [SMILES: Nc1cnccc1-c1nc2ccc(-c3ccccc3)nc2n1-c1ccc(C(=O)O)cn1]

### Synthetic Route:

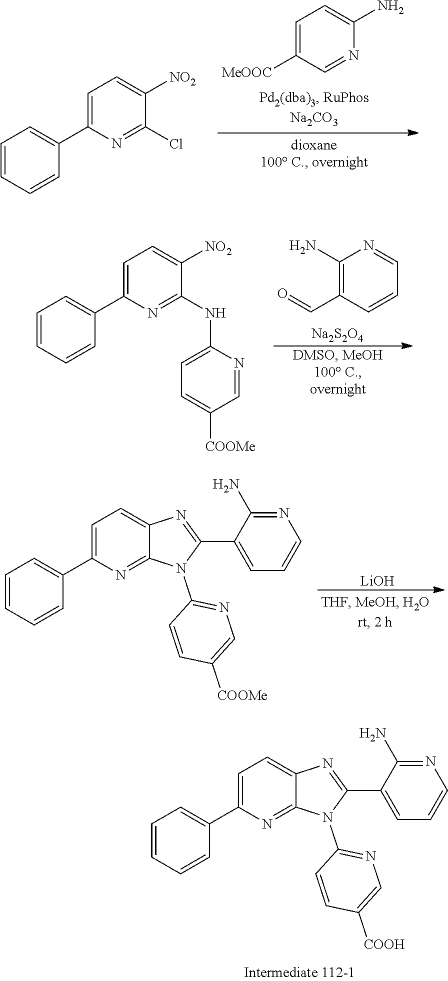

### Step 1: Synthesis of methyl 6-((3-nitro-6-phenylpyridin-2-yl)amino)nicotinate

A solution of methyl 6-aminonicotinate (1.52 g, 1.00 mmol, 1 equiv), 2-chloro-3-nitro-6-phenylpyridine (2.34 g, 1.00 mmol, 1 equiv), tris(dibenzylideneacetone)dipalladium(0) (46 mg, 0.050 mmol, 0.05 equiv), RuPhos (47 mg, 0.1 mmol, 0.1 equiv), and sodium carbonate (2.08 g, 2.00 mmol, 2.0 equiv) in 1,4-dioxane (100 mL) was stirred at 100° C. overnight under nitrogen atmosphere. The mixture was cooled to room temperature and quenched with water (30 mL). The resulting mixture was extracted with ethyl acetate (100 mL×3). The combined organic layers were washed with brine (30 mL), dried over anhydrous sodium sulfate, filtered, and concentrated under reduced pressure. The residue obtained was purified by silica gel chromatography using a 0-50% gradient of ethyl acetate in petroleum ether to afford methyl 6-((3-nitro-6-phenylpyridin-2-yl)amino)nicotinate as a yellow solid (1.8 g, 51%). MS (ESI) calculated for C₁₈H₁₄N₄O₄: 350.10 m/z, found 351.10 [M+H]⁺.

### Step 2: Synthesis of methyl 6-(2-(2-aminopyridin-3-yl)-5-phenyl-3H-imidazo[4,5-b]pyridin-3-yl)nicotinate

A solution of methyl 6-((3-nitro-6-phenylpyridin-2-yl)amino)nicotinate (1.8 g, 5.1 mmol, 1 equiv), 2-aminonicotinaldehyde (0.752 g, 6.17 mmol, 1.20 equiv) and sodium dithionite (2.68 g, 15.4 mmol, 3.0 equiv) in dimethyl sulfoxide (15 mL) and methanol (3 mL) was refluxed at 100° C. overnight. The resulting mixture was cooled to room temperature and quenched with water (50 mL). The mixture was extracted with ethyl acetate (3×100 mL). The organic layers were combined, dried over anhydrous sodium sulfate, filtered, and concentrated in vacuo. The residue obtained was purified by silica gel chromatography using a 0-80% gradient of ethyl acetate in petroleum ether to afford methyl 6-(2-(2-aminopyridin-3-yl)-5-phenyl-3H-imidazo[4,5-b]pyridin-3-yl)nicotinate as a yellow solid (1.5 g, 69%). MS (ESI) calculated for C₂₄H₁₈N₆O₂: 422.15 m/z, found 423.10 [M+H]⁺.

### Step 3: Synthesis of 6-(2-(2-aminopyridin-3-yl)-5-phenyl-3H-imidazo[4,5-b]pyridin-3-yl)nicotinic acid (Intermediate 112-1)

To a solution of methyl 6-(2-(2-aminopyridin-3-yl)-5-phenyl-3H-imidazo[4,5-b]pyridin-3-yl)nicotinate (1.5.0 g, 3.55 mmol, 1 equiv) in tetrahydrofuran (30 mL) and water (30 mL) was added lithium hydroxide (5.0 mL, 2 M in water). The resulting mixture was stirred at room temperature for 1 h. The mixture was concentrated and purified by reverse-phase flash column chromatography on C18 silica gel to afford 6-(2-(2-aminopyridin-3-yl)-5-phenyl-3H-imidazo[4,5-b]pyridin-3-yl)nicotinic acid (Intermediate 112-1) (1.0 g, 69%) as a yellow solid. MS (ESI) calculated for C₂₃H₁₆N₆O₂: 408.13 m/z, found 409.10 [M+H]⁺.

### Example 113: 4-(2-((4-(2-(2-aminopyridin-3-yl)-5-(pyridin-3-yl)-3H-imidazo[4,5-b]pyridin-3-yl)benzyl)amino)ethyl)-2-hydroxybenzaldehyde

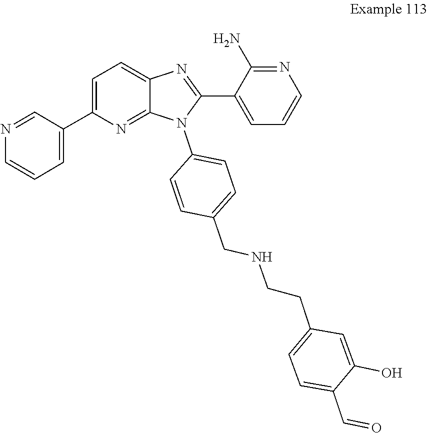 [SMILES: Nc1ncccc1-c1nc2ccc(-c3cccnc3)nc2n1-c1ccc(CNCCc2ccc(C=O)c(O)c2)cc1]

Example 113 was prepared in a manner analogous to Example 19 using Intermediate 113-1 in place of Intermediate 19-3 and Intermediate 67-1 in place of Intermediate 19-2. MS (ESI) calculated for C₃₂H₂₇N₇O₂: 541.61 m/z, found 542.25 [M+H]⁺. ¹H NMR (400 MHz, DMSO-d₆) δ (ppm): 10.19 (s, 1H), 9.26 (s, 1H), 8.65-8.66 (m, 1H), 8.50-8.52 (m, 1H), 8.40-8.42 (m, 1H), 8.14-8.16 (m, 1H), 8.07-8.08 (m, 1H), 7.65-7.68 (m, 7H), 6.89-6.90 (m, 2H), 6.69-6.70 (m, 1H), 4.29 (s, 2H), 3.23-3.25 (m, 2H), 2.95-2.97 (m, 2H).

### Intermediate 113-1: 2-(4-(1,3-dioxolan-2-yl)-3-((4-methoxybenzyl)oxy)phenyl)acetaldehyde

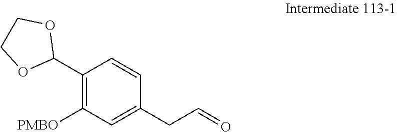

Intermediate 113-1 was prepared in a manner analogous to Intermediate 16-4 using Intermediate 1-3 in place of Intermediate 16-3. MS (ESI) calculated for C₁₉H₂₀O₅: 328.13 m/z, found 329.15 [M+H]⁺.

### Example 114: 4-(2-((4-(2-(2-aminopyridin-3-yl)-3H-imidazo[4,5-b]pyridin-3-yl)benzyl)amino)ethyl)-2-hydroxybenzaldehyde

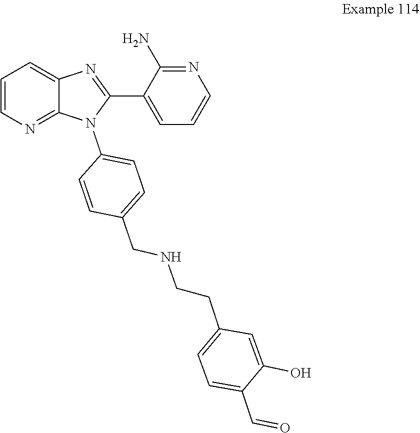 [SMILES: Nc1ncccc1-c1nc2cccnc2n1-c1ccc(CNCCc2ccc(C=O)c(O)c2)cc1]

Example 114 was prepared in a manner analogous to Example 19 using Intermediate 74-1 in place of Intermediate 19-2 and Intermediate 113-1 in place of Intermediate 19-3. MS (ESI) calculated for C₂₇H₂₄N₆O₂: 464.20 m/z, found 465.20 [M+H]⁺. ¹H NMR (300 MHz, DMSO-d₆) δ (ppm): 10.21 (s, 1H), 8.39-8.40 (m, 1H), 8.27-8.29 (m, 1H), 8.05-8.07 (m, 1H), 7.56-7.69 (m, 6H), 7.44-7.48 (m, 1H), 6.89-6.91 (m, 2H), 6.62-6.64 (m, 1H), 4.29 (s, 2H), 3.22-3.27 (m, 2H), 2.95-3.00 (m, 2H).

### Example 115: N-(1-(4-(2-(2-aminopyridin-3-yl)-5-phenyl-3H-imidazo[4,5-b]pyridin-3-yl)phenyl)ethyl)-2-(3-formyl-4-hydroxyphenyl)acetamide

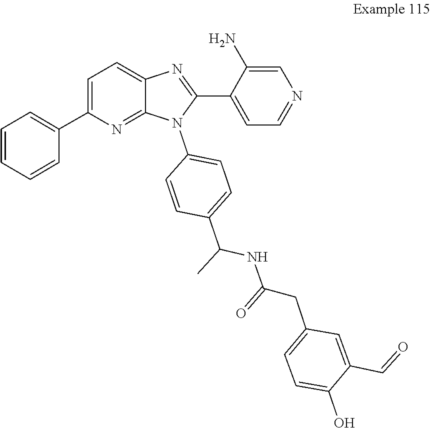 [SMILES: CC(NC(=O)Cc1ccc(O)c(C=O)c1)c1ccc(-n2c(-c3ccncc3N)nc3ccc(-c4ccccc4)nc32)cc1]

Example 115 was prepared in a manner analogous to Example 1 (starting from Step 3) using Intermediate 115-1 in place of the amine starting material and Intermediate 28-2 in place of Intermediate 1-4. MS (ESI) calculated for C₃₄H₂₈N₆O₃: 568.22 m/z, found 569.20 [M+H]⁺. ¹H-NMR (300 MHz, DMSO-d₆) δ (ppm): 10.21 (s, 1H), 8.71 (s, 1H), 8.02-8.25 (m, 4H), 7.44-7.58 (m, 9H), 7.20-7.27 (m, 1H), 6.92-6.95 (m, 1H), 6.37-6.39 (m, 1H), 5.01 (s, 1H), 3.34 (s, 2H), 1.53 (s, 3H).

### Intermediate 115-1: 3-(3-(4-(1-aminoethyl)phenyl)-5-phenyl-3H-imidazo[4,5-b]pyridin-2-yl)pyridin-2-amine

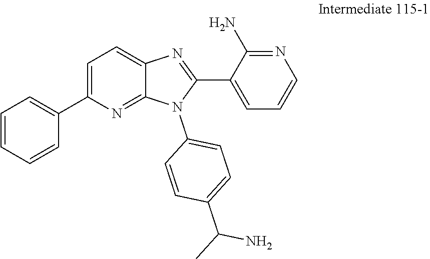 [SMILES: CC(N)c1ccc(-n2c(-c3cccnc3N)nc3ccc(-c4ccccc4)nc32)cc1]

Intermediate 115-1 was prepared in a manner analogous to Intermediate 11-1 using tert-butyl (1-(4-aminophenyl)ethyl)carbamate in place of tert-butyl 2-(4-aminophenyl)acetate. MS (ESI) calculated for C₂₅H₂₂N₆: 406.19 m/z, found: 407.20 [M+H]⁺.

### Example 116: N-(1-{4-[2-(2-aminopyridin-3-yl)-5-phenylimidazo[4,5-b]pyridin-3-yl]phenyl}ethyl)-2-(4-formyl-3-hydroxyphenyl)acetamide

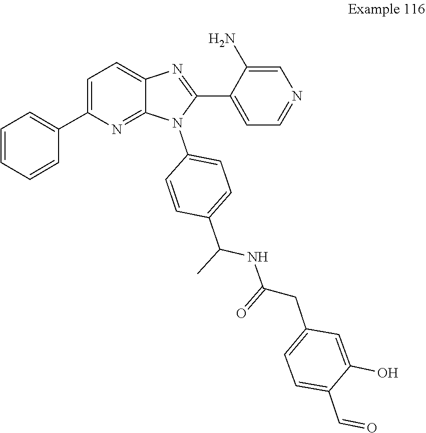 [SMILES: CC(NC(=O)Cc1ccc(C=O)c(O)c1)c1ccc(-n2c(-c3ccncc3N)nc3ccc(-c4ccccc4)nc32)cc1]

Example 116 was prepared in a manner analogous to Example 1 (starting from Step 3) using Intermediate 115-1 in place of the amine starting material. MS (ESI) calculated for C₃₄H₂₈N₆O₃: 568.22 m/z, found 569.15 [M+H]⁺. ¹H NMR (300 MHz, DMSO-d₆) δ (ppm): 10.61-10.75 (m, 1H), 10.08-10.31 (m, 1H), 8.64-8.73 (m, 1H), 8.23-8.37 (m, 1H), 7.93-8.09 (m, 4H), 7.73-7.90 (m, 1H), 7.33-7.65 (m, 10H), 6.81-6.96 (m, 2H), 6.60-6.71 (m, 1H), 4.95-5.11 (m, 1H), 3.40-3.57 (m, 2H), 1.34-1.51 (m, 3H).

### Example 117: N-({4-[2-(2-aminopyridin-3-yl)-5-phenylimidazo[4,5-b]pyridin-3-yl]phenyl}methyl)-3-(4-formyl-3-hydroxyphenyl)propenamide

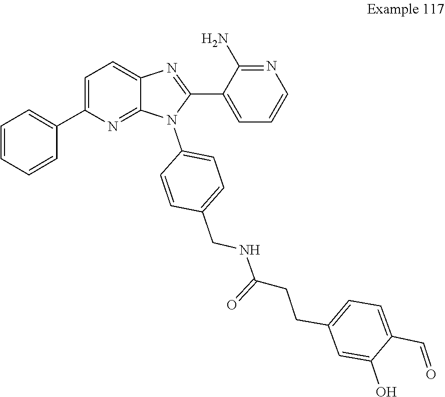 [SMILES: Nc1ncccc1-c1nc2ccc(-c3ccccc3)nc2n1-c1ccc(CNC(=O)CCc2ccc(C=O)c(O)c2)cc1]

Example 117 was prepared in a manner analogous to Example 1 (starting from Step 3) using Intermediate 4-1 in place of the amine starting material and Intermediate 100-2 in place of Intermediate 1-4. MS (ESI) calculated for C₃₄H₂₈N₆O₃: 568.22 m/z, found 569.20 [M+H]⁺. ¹H NMR (300 MHz, DMSO-d₆) δ (ppm): 10.03 (s, 1H), 8.34-8.36 (m, 1H), 8.01-8.06 (m, 4H), 7.75-7.77 (m, 1H), 7.59-7.61 (m, 1H), 7.47-7.56 (m, 3H), 7.38-7.40 (m, 2H), 7.21-7.23 (m, 2H), 6.84-6.91 (m, 2H), 6.80-6.82 (m, 1H), 4.34-4.41 (m, 2H), 2.89-2.93 (m, 2H), 2.49-2.50 (m, 2H).

### Example 118: N-(4-(2-(2-aminopyridin-3-yl)-5-phenyl-3H-imidazo[4,5-b]pyridin-3-yl)phenyl)-3-(4-formyl-3-hydroxyphenyl)propanamide

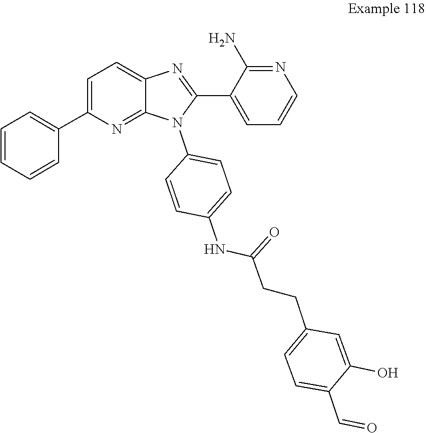 [SMILES: Nc1ncccc1-c1nc2ccc(-c3ccccc3)nc2n1-c1ccc(NC(=O)CCc2ccc(C=O)c(O)c2)cc1]

Example 118 was prepared in a manner analogous to Example 1 (starting from Step 3) using Intermediate 28-1 in place of the amine starting material, Intermediate 100-2 in place of Intermediate 1-4 and T3P in place of PyBOP. MS (ESI) calculated for C₃₃H₂₆N₆O₃: 554.21 m/z, found 555.20 [M+H]⁺. ¹H NMR (400 MHz, DMSO-d₆) δ (ppm): 10.71 (s, 1H), 10.14-10.29 (m, 2H), 8.26-8.35 (m, 1H), 8.01-8.11 (m, 4H), 7.73-7.89 (m, 4H), 7.60-7.65 (m, 2H), 7.40-7.54 (m, 5H), 6.85-6.95 (m, 2H), 6.68-6.75 (m, 1H), 2.90-3.00 (m, 2H), 2.63-2.76 (m, 2H).

### Example 119: 4-{2-[({4-[2-(2-aminopyridin-3-yl)-5-(pyridin-4-yl)imidazo[4,5-b]pyridin-3-yl]phenyl}methyl)amino]ethyl}-2-hydroxybenzaldehyde

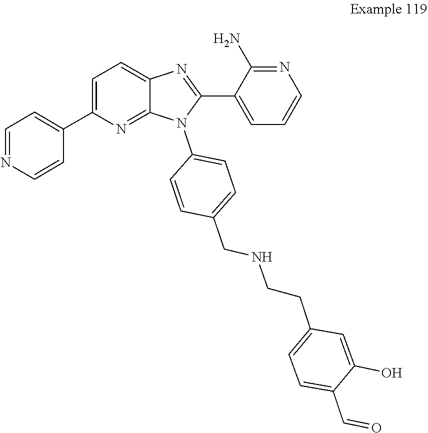 [SMILES: Nc1ncccc1-c1nc2ccc(-c3ccncc3)nc2n1-c1ccc(CNCCc2ccc(C=O)c(O)c2)cc1]

Example 119 was prepared in a manner analogous to Example 19 using Intermediate 119-1 in place of Intermediate 19-2 and Intermediate 113-1 in place of Intermediate 19-3. MS (ESI) calculated for C₃₂H₂₇N₇O₂: 541.22 m/z, found 542.20 [M+H]⁺. ¹H NMR (300 MHz, methanol-d₄) δ (ppm): 8.74 (s, 2H), 8.42-8.46 (m, 3H), 8.32-8.35 (m, 1H), 8.01-8.03 (m, 1H), 7.76-7.78 (m, 2H), 7.67-7.73 (m, 3H), 7.30-7.33 (m, 1H), 6.66-6.79 (m, 3H), 5.57 (s, 1H), 4.38 (s, 2H), 3.35-3.37 (m, 2H), 2.95-3.03 (m, 2H). Note that the aldehyde proton is not visible for some compounds when dissolved in methanol-d₄.

### Intermediate 119-1: 3-(3-(4-(aminomethyl)phenyl)-5-(pyridin-4-yl)-3H-imidazo[4,5-b]pyridin-2-yl)pyridin-2-amine

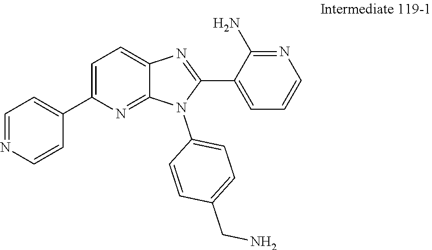 [SMILES: NCc1ccc(-n2c(-c3cccnc3N)nc3ccc(-c4ccncc4)nc32)cc1]

Intermediate 119-1 was prepared in a manner analogous to Intermediate 12-1 using pyridin-4-ylboronic acid in place of (3-morpholinophenyl)boronic acid, [1,1′-bis(di-tert-butylphosphino)ferrocene]dichloropalladium(II)/tribasic potassium phosphate in place of tetrakis(triphenylphosphine)palladium(0)/sodium bicarbonate and 1,4-dioxane in place of toluene/ethanol. MS (ESI) calculated for C₂₃H₁₉N₇: 393.17 m/z, found 394.20 [M+H]⁺.

### Example 120: 4-{2-[({4-[2-(2-aminopyridin-3-yl)-5-phenylimidazo[4,5-b]pyridin-3-yl]phenyl}methyl)amino]ethyl}-3-chloro-2-hydroxybenzaldehyde

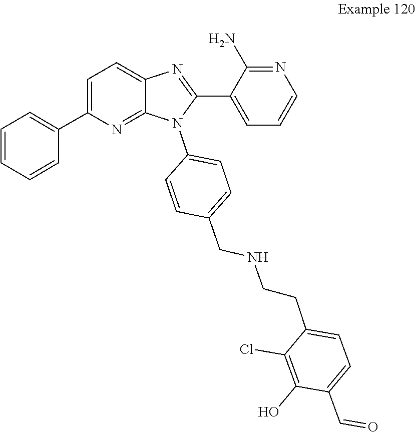 [SMILES: Nc1ncccc1-c1nc2ccc(-c3ccccc3)nc2n1-c1ccc(CNCCc2ccc(C=O)c(O)c2Cl)cc1]

Example 120 was prepared in a manner analogous to Example 19 using Intermediate 4-1 in place of Intermediate 19-2 and Intermediate 120-1 in place of Intermediate 19-3. MS (ESI) calculated for C₃₃H₂₇ClN₆O₂: 574.19 m/z, found 575.20 [M+H]⁺. ¹H NMR (300 MHz, DMSO-d₆) δ (ppm): 11.09-11.46 (m, 1H), 10.14 (s, 1H), 9.05-9.32 (m, 2H), 8.24-8.42 (m, 1H), 7.95-8.17 (m, 4H), 7.63-7.82 (m, 5H), 7.36-7.60 (m, 5H), 7.03-7.20 (m, 1H), 6.44-6.64 (m, 1H), 4.31-4.44 (m, 2H), 3.15-3.37 (m, 4H).

### Intermediate 120-1: 2-(2-chloro-4-(1,3-dioxolan-2-yl)-3-((4-methoxybenzyl)oxy) phenyl)acetaldehyde

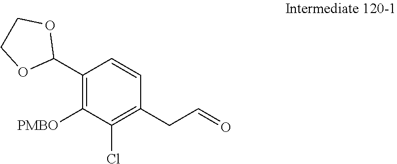

Intermediate 120-1 was prepared in a manner analogous to Intermediate 16-4 using Intermediate 13-1 in place of Intermediate 16-3. MS (ESI) calculated for C₁₉H₁₉ClO₅: 362.09 m/z, found 361.05 [M−H]⁻.

### Example 121: N-{3-[2-(2-aminopyridin-3-yl)-3-[4-({[2-(4-formyl-3-hydroxyphenyl)ethyl]amino}methyl)phenyl]imidazo[4,5-b]pyridin-5-yl]phenyl}acetamide

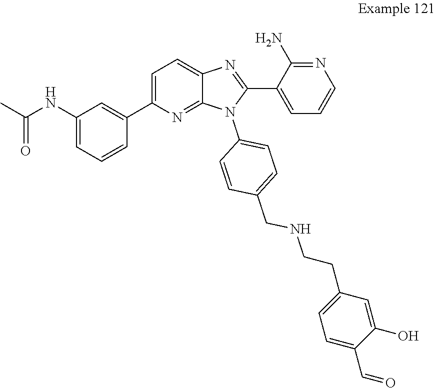 [SMILES: CC(=O)Nc1cccc(-c2ccc3nc(-c4cccnc4N)n(-c4ccc(CNCCc5ccc(C=O)c(O)c5)cc4)c3n2)c1]

Example 121 was prepared in a manner analogous to Example 19 using Intermediate 102-1 in place of Intermediate 19-2 and Intermediate 113-1 in place of Intermediate 19-3. MS (ESI) calculated for C₃₅H₃₁N₇O₃: 597.25 m/z, found 598.25 [M+H]⁺. ¹H NMR (300 MHz, methanol-d₄) δ (ppm): 8.40 (s, 1H), 8.26-8.29 (m, 1H), 7.95-7.98 (m, 2H), 7.65-7.75 (m, 6H), 7.40-7.48 (m, 1H), 7.35-7.39 (m, 1H), 7.29-7.32 (m, 1H), 6.74-6.81 (m, 2H), 6.62-6.69 (m, 1H), 5.57 (s, 1H), 4.36 (s, 2H), 3.34-3.40 (m, 2H), 2.91-3.05 (m, 2H), 2.14 (s, 3H). Note that the aldehyde signal is not visible for some compounds dissolved in methanol-d₄.

### Example 122: 4-{2-[({4-[2-(2-aminopyridin-3-yl)-5-phenylimidazo[4,5-b]pyridin-3-yl]phenyl}methyl)amino]ethyl}-3-fluoro-2-hydroxybenzaldehyde

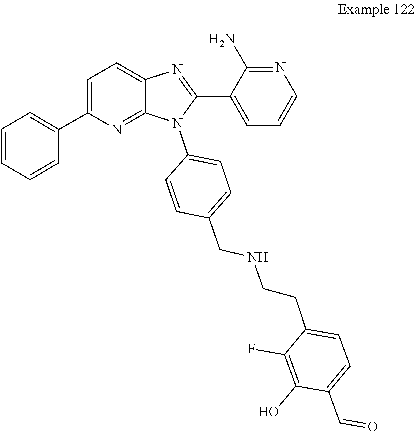 [SMILES: Nc1ncccc1-c1nc2ccc(-c3ccccc3)nc2n1-c1ccc(CNCCc2ccc(C=O)c(O)c2F)cc1]

Example 122 was prepared in a manner analogous to Example 19 using Intermediate 4-1 in place of Intermediate 19-2 and Intermediate 88-1 in place of Intermediate 19-3. MS (ESI) calculated for C₃₃H₂₇FN₆O₂: 558.22 m/z, found 559.20 [M+H]⁺. ¹H NMR (300 MHz, DMSO-d₆) δ (ppm): 10.98-11.02 (s, 1H), 10.25 (s, 1H), 9.10-9.12 (m, 2H), 8.31-8.34 (m, 1H), 7.95-8.05 (m, 4H), 7.60-7.70 (m, 4H), 7.41-7.50 (m, 6H), 6.90-6.95 (m, 1H), 6.54 (s, 1H), 4.32-4.37 (m, 2H), 3.24 (s, 2H), 3.00-3.09 (m, 2H).

### Example 123: 4-(2-((4-(2-(2-aminopyridin-3-yl)-5-phenyl-3H-imidazo[4,5-b]pyridin-3-yl)benzyl)amino)ethyl)-2-fluoro-6-hydroxybenzaldehyde

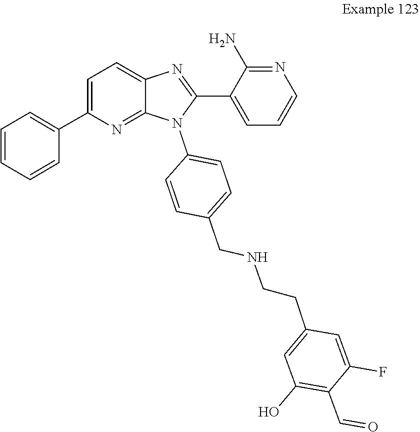 [SMILES: Nc1ncccc1-c1nc2ccc(-c3ccccc3)nc2n1-c1ccc(CNCCc2cc(O)c(C=O)c(F)c2)cc1]

Example 123 was prepared in a manner analogous to Example 19 using Intermediate 4-1 in place of Intermediate 19-2 and Intermediate 123-2 in place of Intermediate 19-3. MS (ESI) calculated for C₃₃H₂₇FN₆O₂: 558.22 m/z, found 559.23 [M+H]⁺. ¹H NMR (300 MHz, DMSO-d₆) δ (ppm) 11.32 (s, 1H), 10.24 (s, 1H), 9.04 (s, 2H), 8.33 (d, J=8.4 Hz, 1H), 8.05-8.00 (m, 4H), 7.73-7.60 (m, 4H), 7.53-7.36 (m, 5H), 6.78 (d, J=10.3 Hz, 2H), 6.56 (dd, J=7.6, 5.3 Hz, 1H), 4.30 (s, 2H), 3.29-3.25 (m, 2H), 2.98 (t, J=7.9 Hz, 2H).

### Intermediate 123-2: 2-(4-(1,3-dioxolan-2-yl)-3-fluoro-5-((4-methoxybenzyl)oxy) phenyl)acetaldehyde

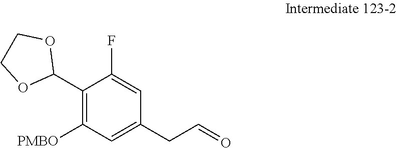

Intermediate 123-2 was prepared in a manner analogous to Intermediate 16-4 using Intermediate 123-1 in place of Intermediate 16-3. MS (ESI) calculated for C₁₉H₁₉FO₅: 346.12 m/z, found 347.14 [M+H]⁺.

### Intermediate 123-1: 2-(4-bromo-2-fluoro-6-((4-methoxybenzyl)oxy)phenyl)-1,3-dioxolane

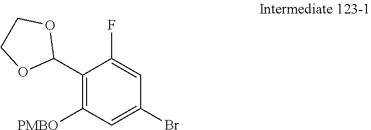

Intermediate 123-1 was prepared in a manner analogous to Intermediate 1-3 (via Intermediate 1-2) starting from 4-bromo-2-fluoro-6-hydroxybenzaldehyde in place of 4-bromo-2-hydroxybenzaldehyde. MS (ESI) calculated for C₁₇H₁₆BrFO₄: 382.02 m/z, found 383.00 [M+H]⁺.

### Example 124: 4-(2-((4-(2-(2-aminopyridin-3-yl)-6-phenyl-3H-imidazo[4,5-b]pyridin-3-yl)benzyl)amino)ethyl)-2-hydroxybenzaldehyde

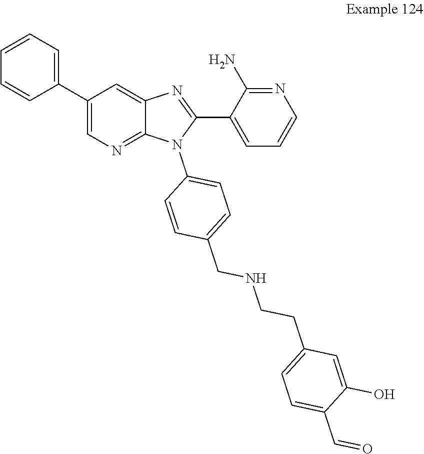 [SMILES: Nc1ncccc1-c1nc2cc(-c3ccccc3)cnc2n1-c1ccc(CNCCc2ccc(C=O)c(O)c2)cc1]

Example 124 was prepared in a manner analogous to Intermediate 19 using Intermediate 124-2 in place of Intermediate 19-2 and Intermediate 113-1 in place of Intermediate 19-3. MS (ESI) calculated for C₃₃H₂₈N₆O₂: 540.62 m/z, found 541.20 [M+H]⁺. ¹H NMR (400 MHz, DMSO-d₆) δ (ppm): 10.20 (s, 1H), 8.69-8.70 (m, 1H), 8.52-8.53 (m, 1H), 8.05-8.07 (m, 1H), 7.79-7.81 (m, 2H), 7.62-7.69 (m, 3H), 7.60-7.62 (m, 3H), 7.55-7.57 (m, 2H), 7.43-7.53 (m, 1H), 6.91-6.93 (m, 2H), 6.64-6.68 (m, 1H), 4.30 (s, 2H), 3.24-3.29 (m, 2H), 2.97-3.00 (m, 2H).

### Intermediate 124-2: 3-(3-(4-(aminomethyl)phenyl)-6-phenyl-3H-imidazo[4,5-b]pyridin-2-yl)pyridin-2-amine

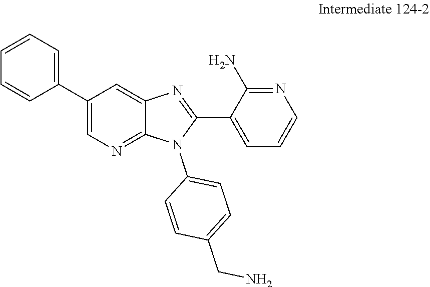 [SMILES: NCc1ccc(-n2c(-c3cccnc3N)nc3cc(-c4ccccc4)cnc32)cc1]

Intermediate 124-2 was prepared in a manner analogous to Intermediate 12-1 using Intermediate 124-1 in place of Intermediate 1-1 and phenyl boronic acid in place of (3-morpholinophenyl)boronic acid. MS (ESI) calculated for C₂₄H₂₀N₆: 392.17 m/z, found 393.15 [M+H]⁺.

### Intermediate 124-1: tert-butyl (4-(2-(2-aminopyridin-3-yl)-6-chloro-3H-imidazo[4,5-b]pyridin-3-yl)benzyl)carbamate

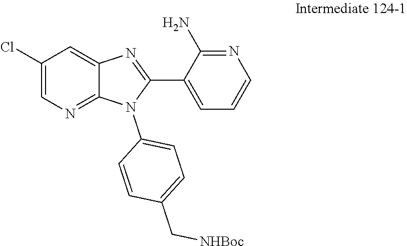 [SMILES: CC(C)(C)OC(=O)NCc1ccc(-n2c(-c3cccnc3N)nc3cc(Cl)cnc32)cc1]

Intermediate 124-1 was prepared in a manner analogous to Intermediate 1-1 using 2,5-dichloro-3-nitropyridine in place of 2,6-dichloro-3-nitropyridine. MS (ESI) calculated for C₂₃H₂₃ClN₆O₂: 450.16 m/z, found 451.15 [M+H]⁺.

### Example 125: 4-(2-((4-(2-(2-aminopyridin-3-yl)-5-phenyl-3H-imidazo[4,5-b]pyridin-3-yl)benzyl)amino)ethyl)benzaldehyde

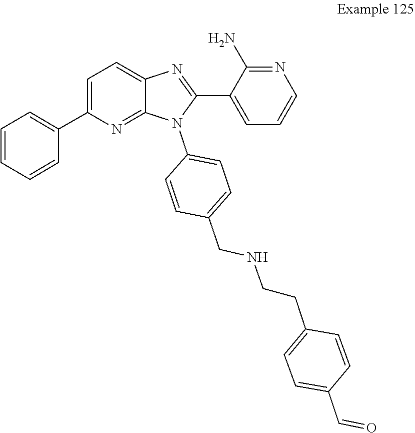 [SMILES: Nc1ncccc1-c1nc2ccc(-c3ccccc3)nc2n1-c1ccc(CNCCc2ccc(C=O)cc2)cc1]

Example 125 was prepared in a manner analogous to Example 19 using Intermediate 4-1 in place of Intermediate 19-2 and Intermediate 125-2 in place of Intermediate 19-3. MS (ESI) calculated for C₃₃H₂₈N₆O: 524.23 m/z, found 525.20 [M+H]⁺. ¹H-NMR (400 MHz, DMSO-d₆) δ (ppm): 9.95 (s, 1H), 8.21-8.28 (m, 1H), 7.84-8.04 (m, 4H), 7.79-7.83 (m, 2H), 7.28-7.57 (m, 10H), 7.17-7.19 (m, 1H), 7.01-7.15 (m, 2H), 6.33-6.37 (m, 1H), 3.86 (s, 2H), 2.68-2.88 (m, 4H).

### Intermediate 125-2: 2-(4-(1,3-dioxolan-2-yl)phenyl)acetaldehyde

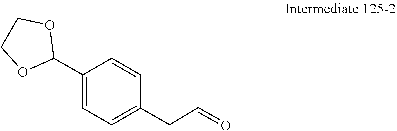 [SMILES: O=CCc1ccc(C2OCCO2)cc1]

Intermediate 125-2 was prepared in a manner analogous to Intermediate 16-4 using Intermediate 125-1 in place of Intermediate 16-3. MS (ESI) calculated for C₁₁H₁₂O₃: 192.08 m/z, found 193.15 [M+H]⁺.

### Intermediate 125-1: 2-(4-bromophenyl)-1,3-dioxolane

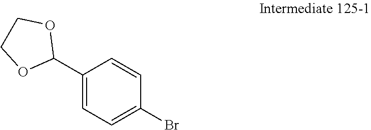 [SMILES: Brc1ccc(C2OCCO2)cc1]

Intermediate 125-1 was prepared in a manner analogous to Intermediate 1-2 using 4-bromobenzaldehyde in place of 4-bromo-2-hydroxybenzaldehyde. MS (ESI) calculated for C₉H₉BrO₂: 227.98 m/z, found 229.00 [M+H]⁺.

### Example 126: 5-(2-((1-(4-(2-(2-aminopyridin-3-yl)-5-phenyl-3H-imidazo[4,5-b]pyridin-3-yl) phenyl) ethyl) amino) ethyl)-2-hydroxybenzaldehyde

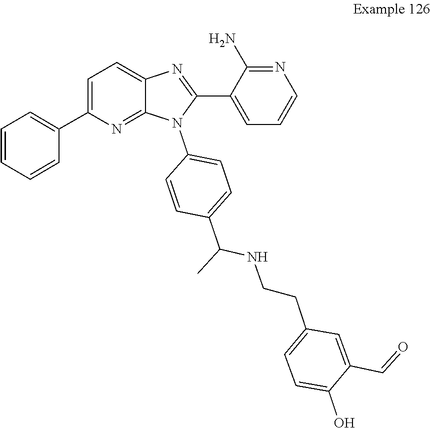 [SMILES: CC(NCCc1ccc(O)c(C=O)c1)c1ccc(-n2c(-c3cccnc3N)nc3ccc(-c4ccccc4)nc32)cc1]

Example 126 was prepared in a manner analogous to Example 19 using Intermediate 115-1 in place of Intermediate 19-2 and Intermediate 92-3 in place of Intermediate 19-3. MS (ESI) calculated for C₃₄H₃₀N₆O₂, 554.24 m/z: found 555.25 [M+H]⁺. ¹H NMR (300 MHz, methanol-d₄) δ (ppm) 10.02 (s, 1H), 8.53 (s, 1H), 8.23 (d, J=8.4 Hz, 1H), 8.08-8.00 (m, 2H), 8.00-7.94 (m, 2H), 7.68-7.52 (m, 4H), 7.48-7.30 (m, 5H), 6.93 (d, J=8.5 Hz, 1H), 6.41 (dd, J=7.7, 5.0 Hz, 1H), 4.30 (d, J=6.7 Hz, 1H), 3.09-2.99 (m, 1H), 2.80-2.99 (m, 3H), 1.65 (d, J=6.7 Hz, 3H).

### Example 127: 4v-(2-((1-(4-(2-(2-aminopyridin-3-yl)-5-phenyl-3H-imidazo[4,5-b]pyridin-3-yl) phenyl) ethyl) amino) ethyl)-2-hydroxybenzaldehyde

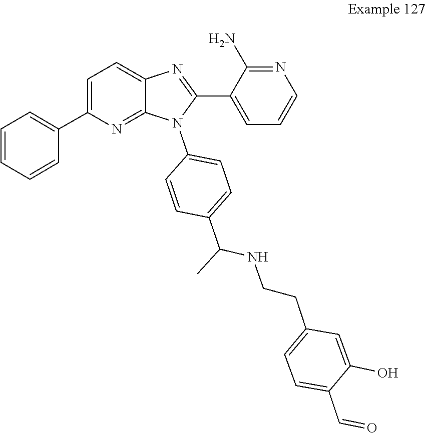 [SMILES: CC(NCCc1ccc(C=O)c(O)c1)c1ccc(-n2c(-c3cccnc3N)nc3ccc(-c4ccccc4)nc32)cc1]

Example 127 was prepared in a manner analogous to Example 19 using Intermediate 115-1 in place of Intermediate 19-2 and Intermediate 113-1 in place of Intermediate 19-3. MS (ESI) calculated for C₃₆H₃₀N₆O₂: 580.26 m/z, found 581.25 [M+H]⁺. ¹H NMR (300 MHz, methanol-d₄) δ (ppm) 8.30 (d, J=8.4 Hz, 1H), 8.09-7.93 (m, 4H), 7.65-7.75 (m, 6H), 7.48-7.38 (m, 3H), 7.32 (d, J=7.8 Hz, 1H), 6.72 (d, J=8.8 Hz, 1H), 6.58-6.50 (m, 1H), 5.59 (s, 1H), 4.61 (d, J=6.8 Hz, 1H), 3.13-2.87 (m, 4H), 1.81 (d, J=6.8 Hz, 3H). Note that the aldehyde signal is not visible for some compounds dissolved in methanol-d₄.

### Example 128: 4-(2-((4-(2-(2-aminopyridin-3-yl)-5-phenyl-3H-imidazo[4,5-b]pyridin-3-yl)benzyl)amino)ethyl)-5-fluoro-2-hydroxybenzaldehyde

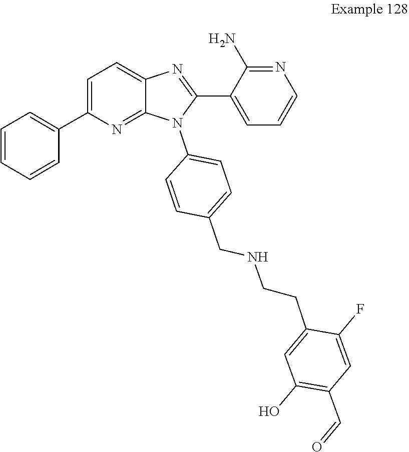 [SMILES: Nc1ncccc1-c1nc2ccc(-c3ccccc3)nc2n1-c1ccc(CNCCc2cc(O)c(C=O)cc2F)cc1]

Example 128 was prepared in a manner analogous to Example 19 using Intermediate 4-1 in place of Intermediate 19-2 and Intermediate 128-3 in place of Intermediate 19-3. MS (ESI) calculated for C₃₃H₂₇FN₆O₂: 558.22 m/z, found 559.25 [M+H]⁺. ¹H NMR (300 MHz, DMSO-d₆) δ (ppm) 10.87 (br s, 1H), 10.23 (d, J=2.7 Hz, 1H), 9.29-9.06 (m, 2H), 8.34 (d, J=8.4 Hz, 1H), 8.12-8.00 (m, 4H), 7.73-7.60 (m, 41H), 7.57-7.34 (m, 6H), 6.98 (d, J=6.2 Hz, 1H), 6.66-6.55 (in 1H), 4.37-4.27 (m, 2H), 3.31-3.17 (m, 2H), 3.07-2.94 (m, 2H).

### Intermediate 128-3: 2-(4-(1,3-dioxolan-2-yl)-2-fluoro-5-((4-methoxybenzyl)oxy) phenyl)acetaldehyde

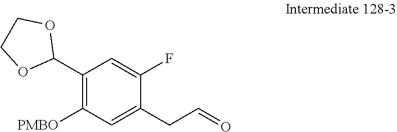

Intermediate 128-3 was prepared in a manner analogous to Intermediate 16-4 using Intermediate 128-2 in place of Intermediate 16-3. MS (ESI) calculated for C₁₉H₁₉FO₅: 346.12 m/z, found 369.00 [M+Na]⁺.

### Intermediate 128-2: 2-(4-bromo-5-fluoro-2-((4-methoxybenzyl)oxy)phenyl)-1,3-dioxolane

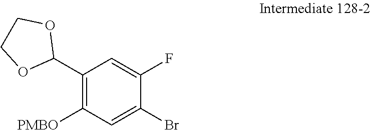

Intermediate 128-2 was prepared in a manner analogous to Intermediate 1-3 (via Intermediate 1-2) starting from Intermediate 128-1 in place of 4-bromo-2-hydroxybenzaldehyde. ¹H NMR (300 MHz, chloroform-d) 6 (ppm) 7.41-7.25 (m, 3H), 7.13 (d, J=5.5 Hz, 1H), 7.01-6.86 (m, 2H), 6.11 (d, J=1.4 Hz, 1H), 5.03 (s, 2H), 4.21-3.95 (m, 4H), 3.84 (s, 3H).

### Intermediate 128-1: 4-bromo-5-fluoro-2-hydroxybenzaldehyde

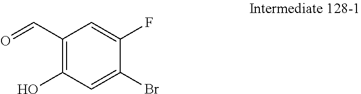 [SMILES: O=Cc1cc(F)c(Br)cc1O]

### Synthetic Route:

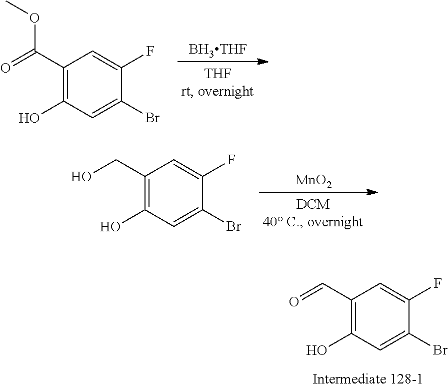

### Step 1: Synthesis of 5-bromo-4-fluoro-2-(hydroxymethyl)phenol

To a cooled (0° C.) solution of methyl 4-bromo-5-fluoro-2-hydroxybenzoate (2.00 g, 8.03 mmol, 1 equiv) in tetrahydrofuran (10 mL) was added borane-tetrahydrofuran complex (1M in tetrahydrofuran, 40 mL, 40 mmol, 5 equiv) and the resulting mixture was stirred at room temperature overnight. The reaction was quenched with ice/water (50 mL) and the resulting mixture was extracted with ethyl acetate (3×80 mL). The organic layers were combined, dried over sodium sulfate, filtered, and concentrated in vacuo to give 5-bromo-4-fluoro-2-(hydroxymethyl) phenol (2 g, crude quant.) as a yellow oil, which was used without further purification in the next step. MS (ESI) calculated for C₇H₆BrFO₂: 219.95 m/z, found 243.90 [M+Na]⁺.

### Step 2: Synthesis of 4-bromo-5-fluoro-2-hydroxybenzaldehyde (Intermediate 128-1)

To a solution of 5-bromo-4-fluoro-2-(hydroxymethyl) phenol (2.00 g, 9.05 mmol, 1 equiv) in dichloromethane (40 mL) was added manganese (IV) oxide (6.29 g, 72.4 mmol, 8 equiv). The reaction mixture was stirred overnight at 40° C. The resulting mixture was cooled to room temperature and concentrated in vacuo. The resulting crude material was purified by silica gel column chromatography using a 0-60% gradient of ethyl acetate in petroleum ether to give 4-bromo-5-fluoro-2-hydroxybenzaldehyde (Intermediate 128-1) (1 g, 50%) as an off-white solid. ¹H NMR (300 MHz, chloroform-d) 6 (ppm) 10.86 (s, 1H), 9.84 (d, J=0.6 Hz, 1H), 7.32 (d, J=7.4 Hz, 1H), 7.30-7.25 (m, 1H).

### Example 129: 5-(3-((4-(2-(2-aminopyridin-3-yl)-5-phenyl-3H-imidazo[4,5-b]pyridin-3-yl)benzyl)amino)propyl)-2-hydroxybenzaldehyde

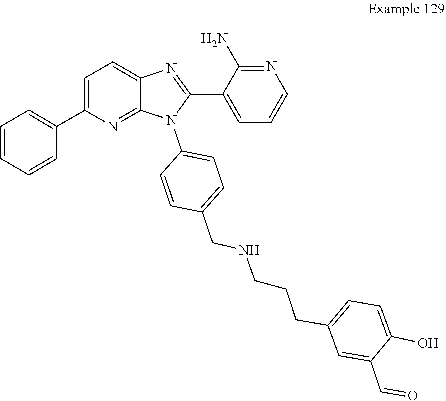 [SMILES: Nc1ncccc1-c1nc2ccc(-c3ccccc3)nc2n1-c1ccc(CNCCCc2ccc(O)c(C=O)c2)cc1]

Example 129 was prepared in a manner analogous to Example 19 using Intermediate 4-1 in place of Intermediate 19-2 and Intermediate 129-1 in place of Intermediate 19-3. MS (ESI) calculated for C₃₄H₃₀N₆O₂: 554.24 m/z, found 555.20 [M+H]⁺. ¹H NMR (300 MHz, DMSO-d₆) δ (ppm) 10.24 (s, 1H), 8.28 (d, J=8.4 Hz, 1H), 8.08-7.95 (m, 4H), 7.59-7.53 (m, 2H), 7.51-7.36 (m, 6H), 7.18 (d, J=7.4 Hz, 1H), 6.96 (d, J=22.5 Hz, 2H), 6.37 (s, 1H), 3.91 (s, 2H), 2.62-2.63 (s, 2H), 2.50-2.46 (m, 2H), 1.79-1.80 (m, 2H).

### Intermediate 129-1: 3-(3-(1,3-dioxolan-2-yl)-4-((4-methoxybenzyl)oxy)phenyl)propanal

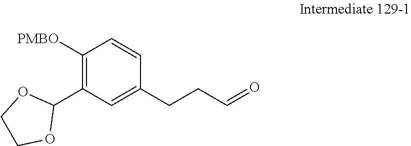

### Synthetic Route:

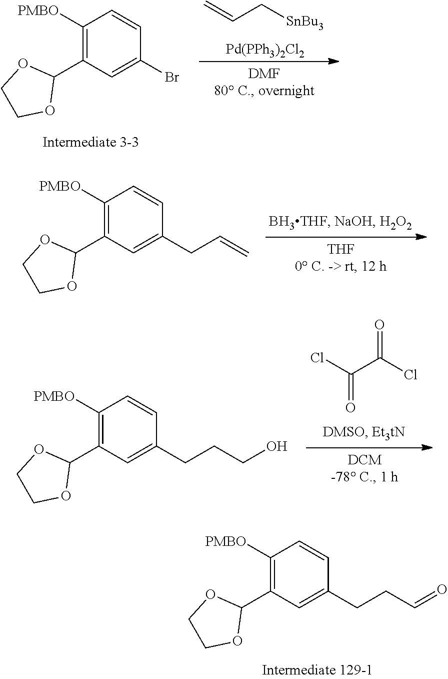

### Step 1: Synthesis of 2-(5-allyl-2-((4-methoxybenzyl)oxy)phenyl)-1,3-dioxolane

A mixture of 2-{5-bromo-2-[(4-methoxyphenyl)methoxy]phenyl}-1,3-dioxolane (Intermediate 3-3) (2.00 g, 5.48 mmol, 1 equiv), tributyl(prop-2-en-1-yl)stannane (3.63 g, 11.0 mmol, 2 equiv) and bis(triphenylphosphine)palladium(II) dichloride (0.38 g, 0.55 mmol, 0.1 equiv) in N,N-dimethylformamide (25 mL) was stirred at 80° C. for 1 h under nitrogen atmosphere. The reaction was quenched with water (50 mL) and extracted with ethyl acetate (3×50 mL). The combined organic layers were washed with brine (50 mL), dried over anhydrous sodium sulfate, filtered, and concentrated in vacuo. The resulting residue was purified by silica gel column chromatography using a 0-75% gradient of ethyl acetate in petroleum ether to afford 2-{2-[(4-methoxyphenyl)methoxy]-5-(prop-2-en-1-yl)phenyl}-1,3-dioxolane (1.5 g, 84%) as a yellow solid. MS (ESI) calculated for C₂₀H₂₂O₄: 326.15 m/z, found 327.20 [M+H]⁺.

### Step 2: Synthesis of 3-(3-(1,3-dioxolan-2-yl)-4-((4-methoxybenzyl)oxy)phenyl)propan-1-ol

To a cooled (0° C.) solution of 2-{2-[(4-methoxyphenyl)methoxy]-5-(prop-2-en-1-yl)phenyl}-1,3-dioxolane (600 mg, 1.84 mmol, 1 equiv) in tetrahydrofuran (25 mL) was added BH₃·tetrahydrofuran (1M in tetrahydrofuran, 2 mL, 2.0 mmol, 1.1 equiv), sodium hydroxide (368 mg, 9.19 mmol, 5 equiv) and hydrogen peroxide (313 mg, 9.19 mmol, 5 equiv). The resulting mixture was stirred at room temperature for 12 h. The reaction was quenched with water (50 mL) and the mixture was extracted with ethyl acetate (3×50 mL). The combined organic layers were washed with brine (50 mL), dried over anhydrous sodium sulfate, filtered, and concentrated in vacuo. The resulting residue was purified by silica gel column chromatography using a 0-54% gradient of ethyl acetate in petroleum ether to afford 3-[3-(1,3-dioxolan-2-yl)-4-[(4-methoxyphenyl)methoxy]phenyl]propan-1-ol (360 mg, 57%) as a light-yellow oil. MS (ESI) calculated for C₂₀H₂₄O₅: 344.16 m/z, found 367.05 [M+Na]⁺.

### Step 3: Synthesis of 3-(3-(1,3-dioxolan-2-yl)-4-((4-methoxybenzyl)oxy)phenyl)propanal (Intermediate 129-1)

To a mixture of oxalyl chloride (193 mg, 1.52 mmol, 1.5 equiv) in dichloromethane (6 mL) at −78° C. was added dimethyl sulfoxide (238 mg, 3.05 mmol, 3 equiv) dropwise over 10 min under nitrogen atmosphere and the resulting mixture was stirred at −78° C. for 0.5 h. A solution of 3-[3-(1,3-dioxolan-2-yl)-4-[(4-methoxyphenyl)methoxy]phenyl]propan-1-ol (350 mg, 1.02 mmol, 1 equiv) in dichloromethane was added dropwise. The mixture was stirred at −78° C. for additional 0.5 h. Triethylamine (514 mg, 5.08 mmol, 5 equiv) was added. The mixture was then allowed to warm to room temperature and the reaction was quenched by the addition of water (20 mL). The resulting mixture was extracted with ethyl acetate (3×50 mL). The combined organic layers were washed with brine (50 mL), dried over anhydrous sodium sulfate, filtered, and concentrated in vacuo. The obtained residue was purified by silica gel column chromatography using a 0-42% gradient of ethyl acetate in petroleum ether to afford 3-[3-(1,3-dioxolan-2-yl)-4-[(4-methoxyphenyl)methoxy]phenyl]propanal (Intermediate 129-1) (180 mg, 52%) as a white solid. MS (ESI) calculated for C₂₀H₂₂O₅: 342.15 m/z, found 365.05 [M+Na]⁺.

### Example 130: 4-(3-((4-(2-(2-aminopyridin-3-yl)-5-phenyl-3H-imidazo[4,5-b]pyridin-3-yl)benzyl)amino)propyl)-2-hydroxybenzaldehyde

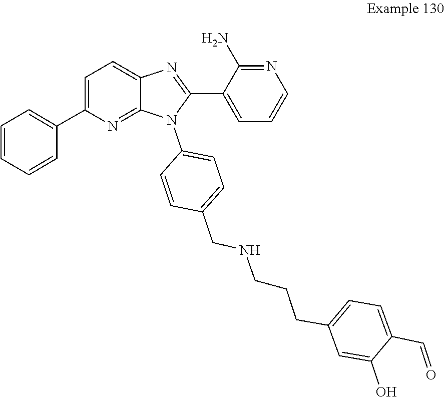 [SMILES: Nc1ncccc1-c1nc2ccc(-c3ccccc3)nc2n1-c1ccc(CNCCCc2ccc(C=O)c(O)c2)cc1]

Example 130 was prepared in a manner analogous to Example 19 using Intermediate 4-1 in place of Intermediate 19-2 and Intermediate 130-1 in place of Intermediate 19-3. MS (ESI): mass calculated for C₃₄H₃₀N₆O₂: 554.24 m/z, found 555.25 [M+H]⁺. ¹H NMR (300 MHz, DMSO-d₆) δ (ppm) 10.81 (s, 1H), 10.19 (s, 1H), 8.96 (s, 2H), 8.34 (d, J=8.4 Hz, 1H), 8.13-8.00 (m, 4H), 7.74-7.57 (m, 5H), 7.56-7.39 (m, 5H), 6.86 (d, J=6.6 Hz, 2H), 6.64 (dd, J=7.6, 5.5 Hz, 1H), 4.28 (d, J=6.3 Hz, 2H), 2.69 (t, J=7.6 Hz, 2H), 2.62-2.56 (m, 2H), 2.05-1.89 (m, 2H).

### Intermediate 130-1: 3-(4-(1,3-dioxolan-2-yl)-3-((4-methoxybenzyl)oxy)phenyl)propanal

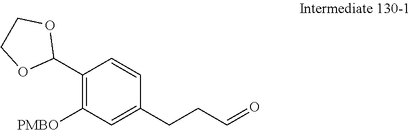

Intermediate 130-1 was prepared in a manner analogous to Intermediate 129-1 starting from Intermediate 1-3 in place of Intermediate 3-3. MS (ESI): mass calculated for C₂₀H₂₂O₅ 342.15 m/z, found 365.10 [M+Na]⁺.

### Example 131: 4-(2-(2-aminopyridin-3-yl)-5-phenyl-3H-imidazo[4,5-b]pyridin-3-yl)-N-(4-formyl-3-hydroxyphenethyl)benzamide

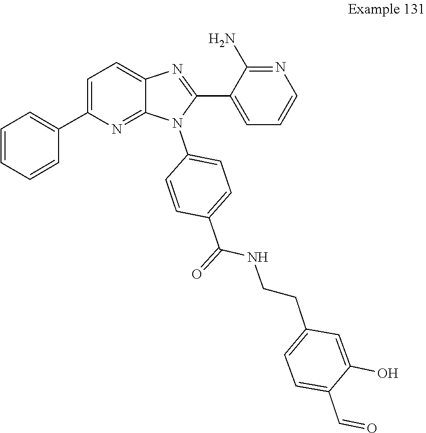 [SMILES: Nc1ncccc1-c1nc2ccc(-c3ccccc3)nc2n1-c1ccc(C(=O)NCCc2ccc(C=O)c(O)c2)cc1]

Example 131 was prepared in a manner analogous to Example 1 (starting from Step 3) using Intermediate 131-2 in place of the amine component, Intermediate 29-2 in place of Intermediate 1-4 and tetrahydrofuran/triethylamine trihydrofluoride in place of 2,2,2-trifluoroacetic acid/methanesulfonic acid. MS (ESI) calculated for C₃₃H₂₆N₆O₃: 554.21 m/z, found 555.15 [M+H]⁺. ¹H NMR (300 MHz, DMSO-d₆) δ (ppm) 10.71 (s, 1H), 10.20 (s, 1H), 8.77 (t, J=5.6 Hz, 1H), 8.35 (d, J=8.4 Hz, 2H), 8.13-8.02 (m, 4H), 8.08-7.93 (m, 2H), 7.77-7.57 (m, 4H), 7.61-7.37 (m, 4H), 6.97-6.81 (m, 2H), 6.75 (dd, J=7.6, 5.7 Hz, 1H), 3.61-3.48 (m, 1H), 2.88 (t, J=7.2 Hz, 2H), 2.21 (s, 1H).

### Intermediate 131-2: 2-(4-((tert-butyldimethylsilyl)oxy)-3-(1,3-dioxolan-2-yl)phenyl)ethan-1-amine

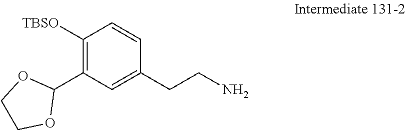 [SMILES: CC(C)(C)[Si](C)(C)Oc1ccc(CCN)cc1C1OCCO1]

Intermediate 131-2 was prepared in a manner analogous to Intermediate 106-1 using Intermediate 131-1 in place of Intermediate 16-3. MS (ESI) calculated for C₁₇H₂₉NO₃Si: 323.19 m/z, found 324.20 [M+H]⁺.

### Intermediate 131-1: (4-bromo-2-(1,3-dioxolan-2-yl)phenoxy)(tert-butyl)dimethylsilane

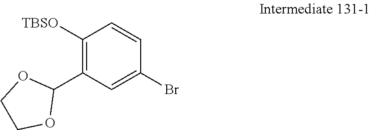 [SMILES: CC(C)(C)[Si](C)(C)Oc1ccc(Br)cc1C1OCCO1]

Intermediate 131-1 was prepared in a manner analogous to Intermediate 16-3 starting from Intermediate 3-2 in place of Intermediate 1-2. MS (ESI) calculated for C₁₅H₂₃BrO₃Si: 358.06 m/z, found 359.05 [M+H]⁺.

### Example 132: N-(4-(2-(2-aminopyridin-3-yl)-5-phenyl-3H-imidazo[4,5-b]pyridin-3-yl)benzyl)-2-(4-formyl-3-hydroxyphenyl)acetamide

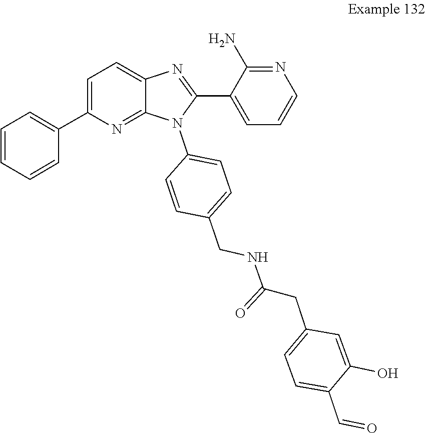 [SMILES: Nc1ncccc1-c1nc2ccc(-c3ccccc3)nc2n1-c1ccc(CNC(=O)Cc2ccc(C=O)c(O)c2)cc1]

Example 132 was prepared in a manner analogous to Example 1 (starting from Step 3) using Intermediate 4-1 in place of the amine starting material. MS (ESI) calculated for C₃₃H₂₆N₆O₃: 554.21 m/z, found 555.20 [M+H]⁺. ¹H NMR (300 MHz, DMSO-d₆) δ (ppm): 10.17 (s, 1H), 8.71-8.74 (m, 1H), 8.24-8.27 (m, 1H), 7.99-8.02 (m, 4H), 7.59-7.61 (m, 1H), 7.40-7.60 (m, 7H), 7.19-7.20 (m, 1H), 6.91 (s, 1H), 6.85-6.89 (m, 1H), 6.39-6.42 (m, 1H), 4.38 (s, 2H), 3.56 (s, 2H).

### Example 133: 4-(2-((4-(2-(2-aminopyridin-3-yl)-5-phenyl-3H-imidazo[4,5-b]pyridin-3-yl)benzyl)amino)ethyl)-2-hydroxybenzaldehyde

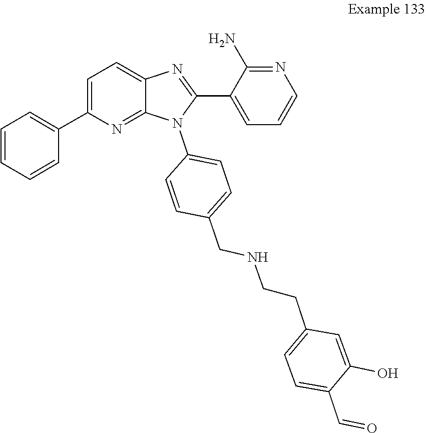 [SMILES: Nc1ncccc1-c1nc2ccc(-c3ccccc3)nc2n1-c1ccc(CNCCc2ccc(C=O)c(O)c2)cc1]

Example 133 was prepared in a manner analogous to Example 16 using Intermediate 4-1 in place of Intermediate 16-2. MS (ESI) calculated for C₃₃H₂₈N₆O₂: 540.23 m/z, found 541.25 [M+H]⁺. ¹H NMR (300 MHz, DMSO-d₆) δ (ppm): 10.19 (s, 1H), 9.10 (s, 1H), 8.25-8.30 (m, 1H), 8.00-8.10 (m, 4H), 7.20-7.70 (m, 9H), 6.85-6.93 (m, 2H), 6.39-6.43 (m, 1H), 4.00-4.16 (m, 2H), 2.90-3.15 (m, 4H).

### Example 134: 4-(2-((1-(4-(2-(2-aminopyridin-3-yl)-5-phenyl-3H-imidazo[4,5-b]pyridin-3-yl)phenyl)cyclobutyl)amino)ethyl)-2-hydroxybenzaldehyde

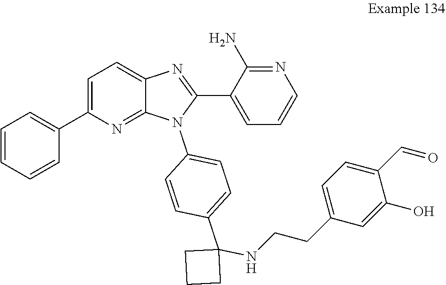 [SMILES: Nc1ncccc1-c1nc2ccc(-c3ccccc3)nc2n1-c1ccc(C2(NCCc3ccc(C=O)c(O)c3)CCC2)cc1]

### Synthetic Route:

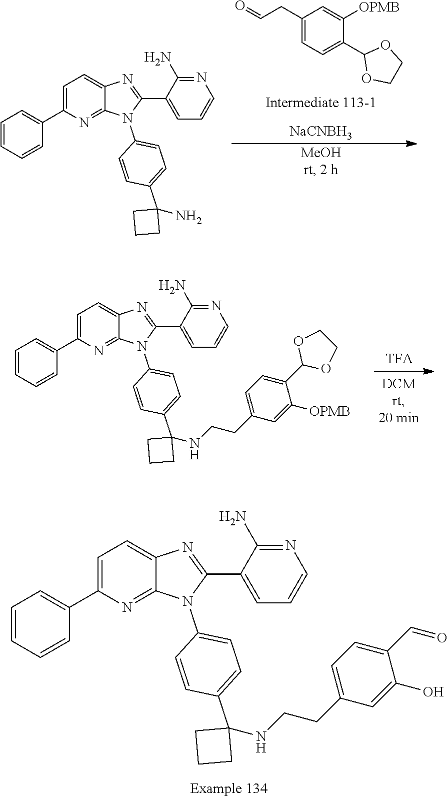

### Step 1: Synthesis of 3-(3-{4-[1-({2-[4-(1,3-dioxolan-2-yl)-3-[(4-methoxyphenyl)methoxy]phenyl]ethyl}amino)cyclobutyl]phenyl}-5-phenylimidazo[4,5-b]pyridin-2-yl)pyridin-2-amine

To a mixture of 3-{3-[4-(1-aminocyclobutyl)phenyl]-5-phenylimidazo[4,5-b]pyridin-2-yl}pyridin-2-amine (80 mg, 0.19 mmol, 1 equiv) and 2-{3-[(tert-butyldimethylsilyl)oxy]-4-(1,3-dioxolan-2-yl)phenyl}acetaldehyde (Intermediate 113-1) (60 mg, 0.19 mmol, 1.0 equiv) in methanol (3 mL) was added sodium cyanoborohydride (58 mg, 0.93 mmol, 5.0 equiv). The mixture was stirred at room temperature for 1 h under nitrogen atmosphere. The reaction was quenched with water (10 mL) and extracted with ethyl acetate (3×10 mL). The combined organic layers were dried over sodium sulfate, filtered, and concentrated in vacuo. The resulting oil was purified by silica gel column chromatography using a 0-20% gradient of methanol in dichloromethane to provide 3-(3-{4-[1-({2-[4-(1,3-dioxolan-2-yl)-3-[(4-methoxyphenyl)methoxy]phenyl]ethyl}amino)cyclobutyl]phenyl}-5-phenylimidazo [4,5-b]pyridin-2-yl)pyridin-2-amine (30 mg, 22%) as a yellow solid. MS (ESI) calculated for C₄₆H₄₄N₆O₄, 744.34 m/z, found 745.30 [M+H]⁺.

### Step 2: Synthesis of 4-{2-[(1-{4-[2-(2-aminopyridin-3-yl)-5-phenylimidazo[4,5-b]pyridin-3-yl]phenyl}cyclobutyl)amino]ethyl}-2-hydroxybenzaldehyde (Example 134)

To a solution of 3-(3-{4-[1-({2-[4-(1,3-Dioxolan-2-yl)-3-[(4-methoxyphenyl)methoxy]phenyl]ethyl}amino)cyclobutyl]phenyl}-5-phenylimidazo[4,5-b]pyridin-2-yl)pyridin-2-amine (30 mg, 0.040 mmol, 1 equiv) in dichloromethane (2.8 mL) was added 2,2,2-trifluoroacetic acid (0.2 mL) and the resulting mixture was stirred at room temperature for 30 min. The resulting mixture was concentrated in vacuo and the resulting oil was purified by preparative HPLC on a XSelect CSH C18 OBD Column using a 10-24% gradient of acetonitrile in water (+0.05% 2,2,2-trifluoroacetic acid) to afford 4-(2-((1-(4-(2-(2-aminopyridin-3-yl)-5-phenyl-3H-imidazo[4,5-b]pyridin-3-yl)phenyl)cyclobutyl)amino)ethyl)-2-hydroxybenzaldehyde (Example 134) (7.0 mg, 25%) as a yellow solid. MS (ESI) calculated for C₃₆H₃₂N₆O₂: 580.26 m/z, found 581.30 [M+H]⁺. ¹H NMR (300 MHz, DMSO-d₆) δ (ppm) 10.00 (s, 1H), 8.30 (d, J=8.4 Hz, 1H), 7.98 (d, J=8.4 Hz, 1H), 7.95-7.89 (m, 2H), 7.86 (dd, J==6.0, 1.7 Hz, 2H), 7.76-7.69 (m, 2H), 7.65-7.53 (m, 3H), 7.41-7.32 (m, 3H), 6.80-6.69 (m, 2H), 6.58-6.49 (m, 1H), 2.82-2.62 (m, 8H), 2.18-2.03 (m, 1H), 1.86-1.71 (m, 1H).

### Example 135: 5-(2-((4-(2-(2-aminopyridin-3-yl)-5-phenyl-3H-imidazo[4,5-b]pyridin-3-yl) benzyl) amino) ethyl)-2-hydroxybenzaldehyde

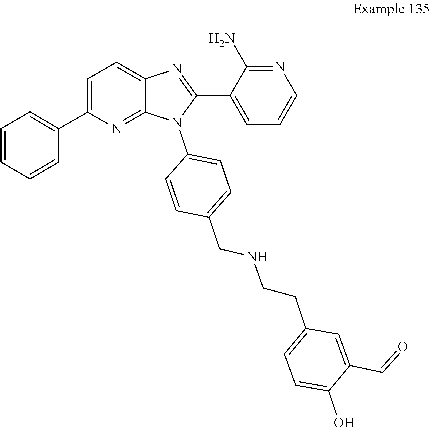 [SMILES: Nc1ncccc1-c1nc2ccc(-c3ccccc3)nc2n1-c1ccc(CNCCc2ccc(O)c(C=O)c2)cc1]

### Synthetic Route:

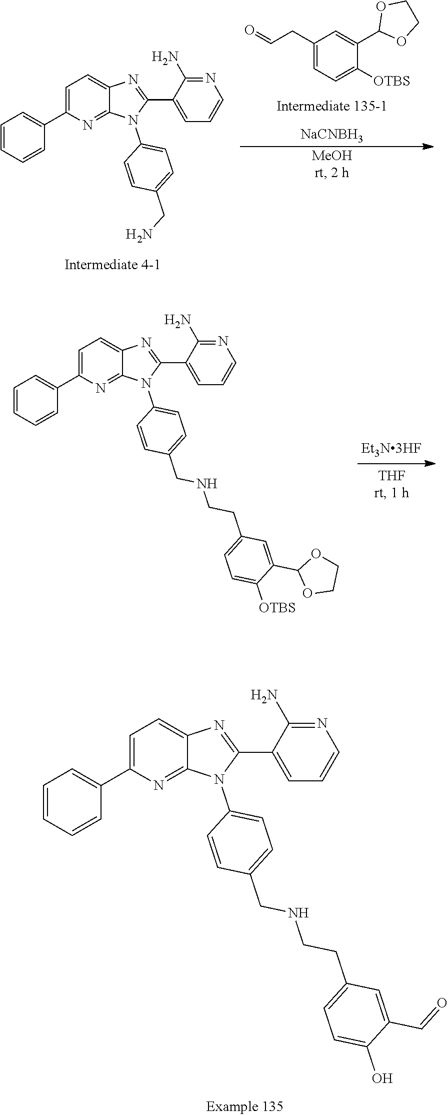 [SMILES: NCc1ccc(-n2c(-c3cccnc3N)nc3ccc(-c4ccccc4)nc32)cc1 ; CC(C)(C)[Si](C)(C)Oc1ccc(CC=O)cc1C1OCCO1 ; "NaCNBH3" ; "MeOHrt, 2 h" ; CC(C)(C)[Si](C)(C)Oc1ccc(CCNCc2ccc(-n3c(-c4cccnc4N)nc4ccc(-c5ccccc5)nc43)cc2)cc1C1OCCO1 ; Nc1ncccc1-c1nc2ccc(-c3ccccc3)nc2n1-c1ccc(CNCCc2ccc(O)c(C=O)c2)cc1]

### Step 1: Synthesis of 3-(3-(4-(((4-((tert-butyldimethylsilyl) oxy)-3-(1,3-dioxolan-2-yl) phenethyl) amino) methyl) phenyl)-5-phenyl-3H-imidazo[4,5-b]pyridin-2-yl) pyridin-2-amine

A mixture of 3-(3-(4-(aminomethyl) phenyl)-5-phenyl-3H-imidazo[4,5-b]pyridin-2-yl) pyridin-2-amine (Intermediate 4-1) (200 mg, 0.51 mmol, 1 equiv), 2-{4-[(tert-butyldimethylsilyl) oxy]-3-(1,3-dioxolan-2-yl) phenyl}acetaldehyde (Intermediate 135-1) (197 mg, 0.612 mmol, 1.2 equiv) and sodium cyanoborohydride (640 mg, 10.2 mmol, 20 equiv) in methanol (10 mL) was stirred at room temperature for 2 h. The reaction was quenched with water (20 mL) and the resulting mixture was extracted with ethyl acetate (20 mL×3). The combined organic extracts were washed with brine, dried over anhydrous sodium sulfate, filtered, and concentrated in vacuo. The resulting residue was purified by flash column chromatography on silica gel using a 0-100% gradient of ethyl acetate in petroleum ether to afford 3-(3-(4-(((4-((tert-butyldimethylsilyl) oxy)-3-(1,3-dioxolan-2-yl) phenethyl) amino) methyl) phenyl)-5-phenyl-3H-imidazo[4,5-b]pyridin-2-yl) pyridin-2-amine (192 mg, 54%) as a white oil. MS (ESI) calculated for C₄₁H₄₆N₆O₃Si: 698.94 m/z, found 699.15 [M+H]⁺

### Step 2: Synthesis of 5-(2-((4-(2-(2-aminopyridin-3-yl)-5-phenyl-3H-imidazo[4,5-b]pyridin-3-yl) benzyl) amino) ethyl)-2-hydroxybenzaldehyde (Example 135)

Triethylamine trihydrofluoride (1 mL) was added to a solution of 3-(3-(4-(((4-((tert-butyldimethylsilyl) oxy)-3-(1,3-dioxolan-2-yl) phenethyl) amino) methyl) phenyl)-5-phenyl-3H-imidazo[4,5-b]pyridin-2-yl) pyridin-2-amine (90 mg, 0.14 mmol, 1 equiv) in tetrahydrofuran (10 mL). The mixture was stirred at room temperature for 1 h. The mixture was concentrated in vacuo and the resulting residue was purified by preparative HPLC on a XSelect CSH Fluoro Phenyl column using a 8-38% gradient of acetonitrile in water (+0.05% 2,2,2-trifluoroacetic acid) to afford 5-(2-((4-(2-(2-aminopyridin-3-yl)-5-phenyl-3H-imidazo[4,5-b]pyridin-3-yl) benzyl) amino) ethyl)-2-hydroxybenzaldehyde (Example 135) (5.9 mg, 5.94%) as an off-white solid. MS (ESI) calculated for C₃₃H₂₈N₆O₂: 540.23 m/z, found 541.25 [M+H]⁺. ¹H NMR (300 MHz, methanol-d₄) δ (ppm) 8.31 (d, J=8.5 Hz, 1H), 8.15-7.89 (m, 4H), 7.89-7.61 (m, 5H), 7.51-7.30 (m, 4H), 6.86-6.64 (m, 3H), 5.60 (s, 1H), 4.39 (s, 2H), 3.03 (s, 2H), 2.98 (d, J=7.9 Hz, 2H). Note that the aldehyde signal is not visible for some compounds dissolve in methanol-d₄.

### Intermediate 135-1: 2-(4-((tert-butyldimethylsilyl)oxy)-3-(1,3-dioxolan-2-yl)phenyl) acetaldehyde

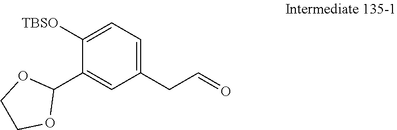 [SMILES: CC(C)(C)[Si](C)(C)Oc1ccc(CC=O)cc1C1OCCO1]

Intermediate 135-1 was prepared in a manner analogous to Intermediate 16-4 using Intermediate 131-1 in place of Intermediate 16-3. MS (ESI) calculated for C₁₇H₂₆O₄Si: 322.16 m/z, found 323.15 [M+H]⁺.

### Example 136: 5-(1-(4-(2-(2-aminopyridin-3-yl)-5-phenyl-3H-imidazo[4,5-b]pyridin-3-yl) benzyl) piperidin-4-yl)-2-hydroxybenzaldehyde

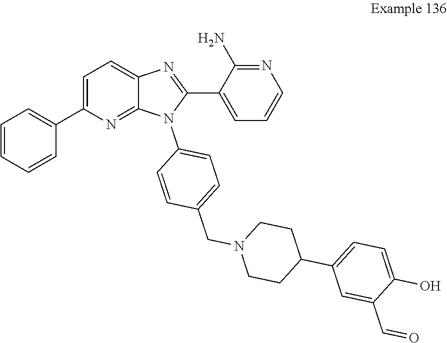 [SMILES: Nc1ncccc1-c1nc2ccc(-c3ccccc3)nc2n1-c1ccc(CN2CCC(c3ccc(O)c(C=O)c3)CC2)cc1]

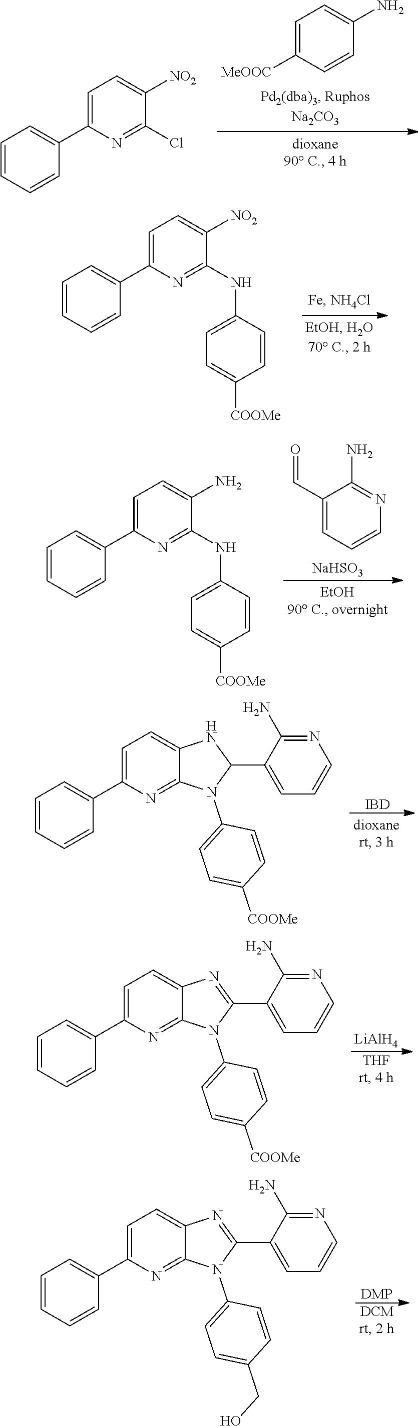

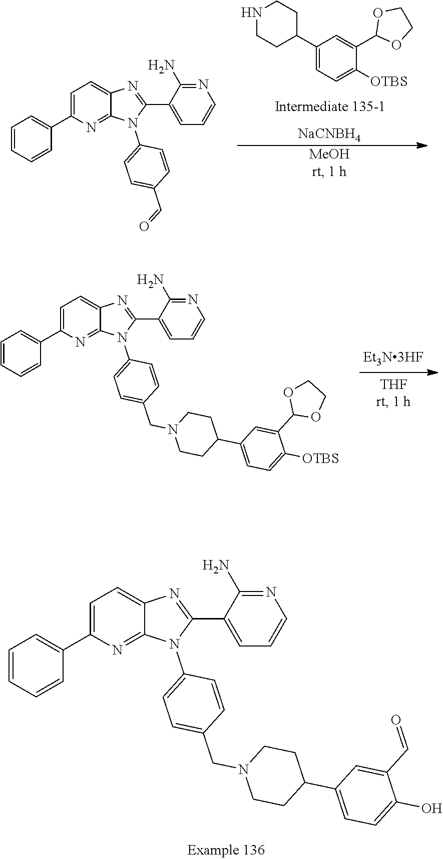 [SMILES: Nc1ncccc1-c1nc2ccc(-c3ccccc3)nc2n1-c1ccc(C=O)cc1 ; CC(C)(C)[Si](C)(C)Oc1ccc(C2CCNCC2)cc1C1OCCO1 ; "NaCNBH4" ; "MeOHrt, 1 h" ; CC(C)(C)[Si](C)(C)Oc1ccc(C2CCN(Cc3ccc(-n4c(-c5cccnc5N)nc5ccc(-c6ccccc6)nc54)cc3)CC2)cc1C1OCCO1 ; Nc1ncccc1-c1nc2ccc(-c3ccccc3)nc2n1-c1ccc(CN2CCC(c3ccc(O)c(C=O)c3)CC2)cc1]

### Step 1: Synthesis of methyl 4-((3-nitro-6-phenylpyridin-2-yl) amino) benzoate

A mixture of 2-chloro-3-nitro-6-phenylpyridine (10.0 g, 42.6 mmol, 1 equiv), methyl 4-aminobenzoate (7.73 g, 51.1 mmol, 1.2 equiv), tris(dibenzylideneacetone)dipalladium(0) (3.90 g, 4.26 mmol, 0.1 equiv), RuPhos (1.99 g, 4.26 mmol, 0.1 equiv) and sodium carbonate (9.03 g, 85.2 mmol, 2.0 equiv) in 1,4-dioxane (200 mL) was stirred for 4 h at 90° C. under nitrogen atmosphere. The mixture was cooled to room temperature and quenched with water (200 mL). The resulting mixture was extracted with ethyl acetate (100 mL×3). The combined organic layers were washed with brine (100 mL), dried over anhydrous sodium sulfate, filtered, and concentrated in vacuo. The resulting residue was purified by silica gel column chromatography using a 0-100% gradient of ethyl acetate in petroleum ether to afford methyl 4-[(3-nitro-6-phenylpyridin-2-yl) amino]benzoate (14.0 g, 81%) as a red/brown solid. MS (ESI) calculated for C₁₉H₁₅N₃O₄: 349.11 m/z, found 350.05 [M+H]⁺.

### Step 2: Synthesis of methyl 4-((3-amino-6-phenylpyridin-2-yl) amino) benzoate

To a stirred mixture of methyl 4-[(3-nitro-6-phenylpyridin-2-yl) amino]benzoate (14.0 g, 40.1 mmol, 1.0 equiv) in ethanol (100 mL) and saturated aqueous ammonium chloride (100 mL) was added iron (11.19 g, 200.4 mol, 5.0 equiv) in portions under nitrogen atmosphere and the resulting mixture was stirred for 2 h at 70° C. The mixture was cooled to room temperature and extracted with ethyl acetate (100 mL×3). The combined organic layers were washed with brine (100 mL), dried over anhydrous sodium sulfate, filtered, and concentrated in vacuo. The resulting residue was purified by silica gel column chromatography using a 0-50% gradient of ethyl acetate in petroleum ether to provide methyl 4-[(3-amino-6-phenylpyridin-2-yl) amino]benzoate (11.0 g, 85%) as a yellow solid. MS (ESI) calculated for C₁₉H₁₇N₃O₂: 319.13 m/z, found 320.05 [M+H]⁺.

### Step 3: Synthesis of methyl 4-(2-(2-aminopyridin-3-yl)-5-phenyl-1,2-dihydro-3H-imidazo[4,5-b]pyridin-3-yl)benzoate

To a stirred solution of methyl 4-[(3-amino-6-phenylpyridin-2-yl) amino]benzoate (11 g, 34 mmol, 1.0 equiv) and 2-aminopyridine-3-carbaldehyde (4.63 g, 37.9 mmol, 1.1 equiv) in anhydrous ethanol (350 mL) was added sodium bisulfite (10.75 g, 103.3 mmol, 3 equiv). The resulting mixture was stirred overnight at 90° C. The resulting mixture was cooled to room temperature and quenched with water (300 mL). The precipitated solids were collected by filtration and washed with ethyl acetate (3×50 mL). The resulting product was dried to afford methyl 4-(2-(2-aminopyridin-3-yl)-5-phenyl-1,2-dihydro-3H-imidazo[4,5-b]pyridin-3-yl)benzoate (12.0 g, 82%) as an orange solid. MS (ESI) calculated for C₂₅H₂₁N₅O₂: 423.17 m/z, found 424.25 [M+H]⁺.

### Step 4: Synthesis of methyl 4-(2-(2-aminopyridin-3-yl)-5-phenyl-3H-imidazo[4,5-b]pyridin-3-yl) benzoate

To a stirred solution of methyl 4-(2-(2-aminopyridin-3-yl)-5-phenyl-1,2-dihydro-3H-imidazo[4,5-b]pyridin-3-yl)benzoate (12.0 g, 28.3 mmol, 1.0 equiv) in anhydrous 1,4-dioxane was added (diacetoxyiodo)benzene (27.38 g, 85.01 mmol, 3 equiv) at room temperature and the resulting mixture was stirred at room temperature for 3 h. The reaction was quenched with water (200 mL) and the precipitated solids were collected by filtration and dried by air to afford methyl 4-(2-(2-aminopyridin-3-yl)-5-phenyl-3H-imidazo[4,5-b]pyridin-3-yl) benzoate (11.0 g, 91%) as a yellow solid. MS (ESI) calculated for C₂₅H₁₉N₅O₂: 421.15 m/z, found 422.10 [M+H]⁺.

### Step 5: Synthesis of (4-(2-(2-aminopyridin-3-yl)-5-phenyl-3H-imidazo[4,5-b]pyridin-3-yl) phenyl) methanol

To a cooled (0° C.) solution of methyl 4-(2-(2-aminopyridin-3-yl)-5-phenyl-3H-imidazo[4,5-b]pyridin-3-yl) benzoate (1.00 g, 2.37 mmol, 1 equiv) in tetrahydrofuran (70 mL) was added dropwise lithium aluminum hydride (2.5 M in tetrahydrofuran, 2.8 mL, 7.2 mmol) under nitrogen atmosphere. The mixture was stirred for 4 h at room temperature. The reaction was quenched with saturated aqueous ammonium chloride (40 mL) at 0° C. and the resulted mixture was extracted with chloroform/isopropanol (3:1, 2×40 mL). The combined organic extracts were washed with brine (50 mL), dried over anhydrous sodium sulfate, filtered, and concentrated in vacuo to give (4-(2-(2-aminopyridin-3-yl)-5-phenyl-3H-imidazo[4,5-b]pyridin-3-yl) phenyl) methanol (900 mg, 82%) as a brown solid. MS (ESI) calculated for C₂₄H₁₉N₅O: 393.16 m/z, found 394.25 [M+H]⁺.

### Step 6: Synthesis of 4-(2-(2-aminopyridin-3-yl)-5-phenyl-3H-imidazo[4,5-b]pyridin-3-yl) benzaldehyde

To a cooled (0° C.) solution of (4-(2-(2-aminopyridin-3-yl)-5-phenyl-3H-imidazo[4,5-b]pyridin-3-yl) phenyl) methanol (950 mg, 2.42 mmol, 1 equiv) in anhydrous dichloromethane (100 mL) was added Dess-Martin periodinane (1.23 g, 2.90 mmol, 1.2 equiv) and the mixture was stirred for 2 h at room temperature. The reaction mixture was cooled to 0° C. and quenched with saturated aqueous sodium bicarbonate. The resulting mixture was extracted with dichloromethane (100 mL×2). The combined organic layers were washed with brine (100 mL), dried over anhydrous sodium sulfate, filtered, and concentrated in vacuo. The crude residue was purified by silica gel column chromatography using a 0-5% gradient of methanol in dichloromethane to afford 4-(2-(2-aminopyridin-3-yl)-5-phenyl-3H-imidazo[4,5-b]pyridin-3-yl) benzaldehyde (650 mg, 65%) as a brown solid. MS (ESI) calculated for C₂₄H₁₇N₅O: 391.14 m/z, found 392.25 [M+H]⁺.

### Step 7: Synthesis of 3-(3-(4-((4-(4-((tert-butyl dimethylsilyl) oxy)-3-(1,3-dioxolan-2-yl) phenyl) piperidin-1-yl) methyl) phenyl)-5-phenyl-3H-imidazo[4,5-b]pyridin-2-yl) pyridin-2-amine

A mixture of 4-(2-(2-aminopyridin-3-yl)-5-phenyl-3H-imidazo[4,5-b]pyridin-3-yl) benzaldehyde (200 mg, 0.48 mmol, 1 equiv), 4-(4-((tert-butyldimethylsilyl) oxy)-3-(1,3-dioxolan-2-yl) phenyl) piperidine (Intermediate 136-1) (188 mg, 0.524 mmol, 1.1 equiv) and sodium cyanoborohydride (299 mg, 4.76 mmol, 10 equiv) in methanol (10 mL) was stirred at room temperature for 1 hour. The reaction was quenched with water (20 mL) and extracted with ethyl acetate (20 mL×3). The combined organic extracts were washed with brine, filtered, and concentrated in vacuo. The resulting residue was purified by flash column chromatography on silica gel using a 0-100% gradient of ethyl acetate in petroleum ether to afford 3-(3-(4-((4-(4-((tert-butyl dimethylsilyl) oxy)-3-(1,3-dioxolan-2-yl) phenyl) piperidin-1-yl) methyl) phenyl)-5-phenyl-3H-imidazo[4,5-b]pyridin-2-yl) pyridin-2-amine (171 mg, 49%) as a white solid. MS (ESI) calculated for C₄₄H₅₀N₆O₃Si: 738.37 m/z, found 739.40 [M+H]⁺.

### Step 8: Synthesis of 5-(1-(4-(2-(2-aminopyridin-3-yl)-5-phenyl-3H-imidazo[4,5-b]pyridin-3-yl) benzyl) piperidin-4-yl)-2-hydroxybenzaldehyde (Example 136)

Triethylamine trihydrofluoride (1 mL) was added to a solution of 3-(3-(4-((4-(4-((tert-butyl dimethylsilyl) oxy)-3-(1,3-dioxolan-2-yl) phenyl) piperidin-1-yl) methyl) phenyl)-5-phenyl-3H-imidazo[4,5-b]pyridin-2-yl) pyridin-2-amine (100 mg, 0.13 mmol, 1 equiv) in tetrahydrofuran (10 mL). The mixture was stirred at room temperature for 1 hour. The mixture was concentrated in vacuo and the resulting residue was purified by preparative HPLC on a XBridge Prep OBD C18 Column using a 17-42% gradient of acetonitrile in water (+10 mmol/L ammonium bicarbonate) to afford 5-(1-(4-(2-(2-aminopyridin-3-yl)-5-phenyl-3H-imidazo[4,5-b]pyridin-3-yl) benzyl) piperidin-4-yl)-2-hydroxybenzaldehyde (Example 136) (10.3 mg, 10.96%) as an off-white solid. MS (ESI) calculated for C₃₆H₃₁N₆O₂: 580.26 m/z, found 581.25 [M+H]⁺. ¹H NMR (300 MHz, methanol-d₄) δ (ppm) 8.30 (d, J=8.5 Hz, 1H), 8.10-7.98 (m, 4H), 7.85-7.69 (m, 4H), 7.69-7.62 (m, 1H), 7.55-7.36 (m, 4H), 7.28 (s, 1H), 7.11 (d, J=8.3 Hz, 1H), 6.82 (dd, J=13.6, 8.5 Hz, 1H), 6.71 (dd, J=7.7, 5.7 Hz, 1H), 5.62 (d, J=6.5 Hz, 1H), 4.49 (s, 2H), 3.69 (d, J=12.0 Hz, 2H), 3.23 (d, J=12.5 Hz, 2H), 2.89 (d, J=15.5 Hz, 1H), 2.14 (d, J=14.4 Hz, 2H), 1.98 (t, J=12.9 Hz, 2H). Note that the aldehyde signal is not visible for some compounds dissolved in methanol-d₄.

### Intermediate 136-1: 4-(4-((tert-butyldimethylsilyl)oxy)-3-(1,3-dioxolan-2-yl)phenyl)piperidine

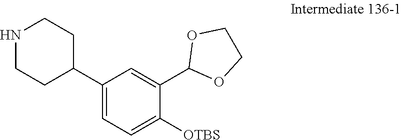 [SMILES: CC(C)(C)[Si](C)(C)Oc1ccc(C2CCNCC2)cc1C1OCCO1]

### Synthetic Route:

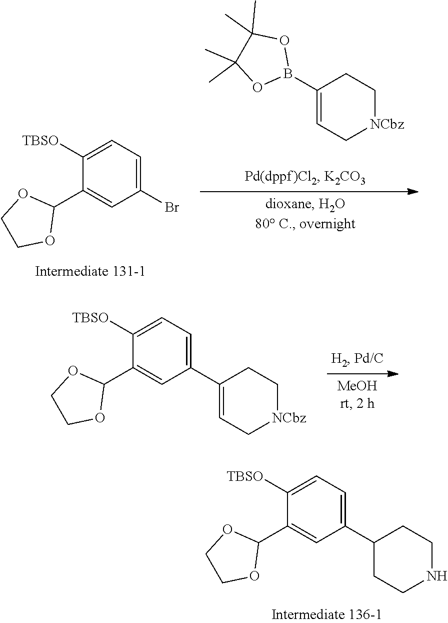

### Step 1: Synthesis of benzyl 4-(4-((tert-butyldimethylsilyl)oxy)-3-(1,3-dioxolan-2-yl)phenyl)piperidine-1-carboxylate

A mixture of 4-bromo-2-(1,3-dioxolan-2-yl)phenoxy(tert-butyl)dimethylsilane (Intermediate 131-1) (500 mg, 1.39 mmol, 1 equiv), benzyl 4-(4,4,5,5-tetramethyl-1,3,2-dioxaborolan-2-yl)-3,6-dihydro-2H-pyridine-1-carboxylate (525 mg, 1.53 mmol, 1.1 equiv), potassium carbonate (577 mg, 4.17 mmol, 3 equiv) and [1,1′-bis(diphenylphosphino)ferrocene]dichloropalladium(II) (102 mg, 0.139 mmol, 0.1 equiv) in 1,4-dioxane (15 mL) and water (0.1 mL) was stirred at 80° C. for 16 h under nitrogen atmosphere. The reaction mixture was cooled to room temperature and quenched with water (20 mL). The resulting mixture was extracted with ethyl acetate (20 mL×3). The combined organic extracts were washed with brine, dried over anhydrous sodium sulfate, filtered, and concentrated under reduced pressure. The resulting residue was purified by silica gel column chromatography using a 0-100% gradient of ethyl acetate in petroleum ether to afford benzyl 4-(4-((tert-butyldimethylsilyl)oxy)-3-(1,3-dioxolan-2-yl)phenyl)piperidine-1-carboxylate (300 mg, 43%) as a yellow solid. MS (ESI) calculated for C₂₈H₃₉NO₅Si: 497.26 m/z, found 498.20 [M+H]⁺.

### Step 2: Synthesis of 4-(4-((tert-butyldimethylsilyl)oxy)-3-(1,3-dioxolan-2-yl)phenyl) piperidine (Intermediate 136-1)

A solution of benzyl 4-(4-((tert-butyldimethylsilyl)oxy)-3-(1,3-dioxolan-2-yl)phenyl)piperidine-1-carboxylate (200 mg, 0.403 mmol, 1 equiv) in methanol (10 mL) was treated with 10% palladium on carbon (15 mg, 0.14 mmol, 0.35 equiv) and the resulting mixture was stirred at room temperature under hydrogen atmosphere for 2 h. The resulting mixture was filtered rinsing with methanol (20 mL×5). The filtrate was concentrated under reduced pressure to afford 4-(4-((tert-butyldimethylsilyl)oxy)-3-(1,3-dioxolan-2-yl)phenyl)piperidine (Intermediate 136-1) (120 mg, 812%) as yellow oil, which was used in subsequent transformations without further purification. MS (ESI) calculated for C₂₀H₃₃NO₃Si: 363.22 m/z, found 364.20 [M+H]⁺.

### Example 137: 4-(2-(2-aminopyridin-3-yl)-5-phenyl-3H-imidazo[4,5-b]pyridin-3-yl)-N-(4-formyl-3-hydroxybenzyl)benzamide

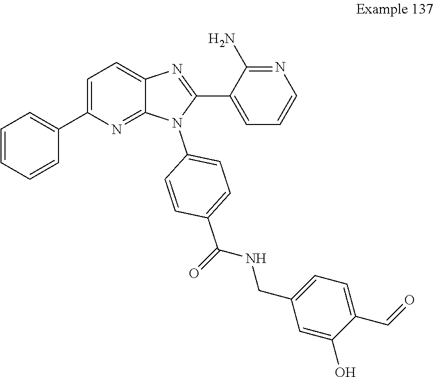 [SMILES: Nc1ncccc1-c1nc2ccc(-c3ccccc3)nc2n1-c1ccc(C(=O)NCc2ccc(C=O)c(O)c2)cc1]

### Synthetic Route:

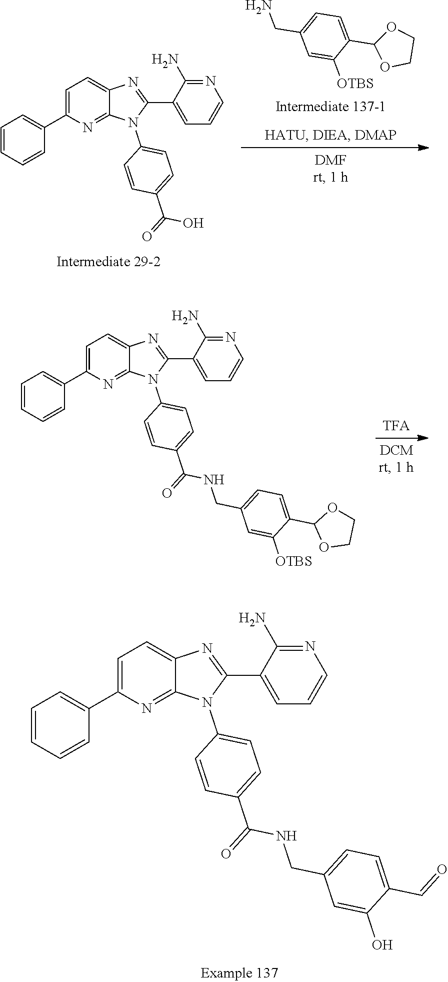 [SMILES: Nc1ncccc1-c1nc2ccc(-c3ccccc3)nc2n1-c1ccc(C(=O)O)cc1 ; CC(C)(C)[Si](C)(C)Oc1cc(CN)ccc1C1OCCO1 ; CC(C)(C)[Si](C)(C)Oc1cc(CNC(=O)c2ccc(-n3c(-c4cccnc4N)nc4ccc(-c5ccccc5)nc43)cc2)ccc1C1OCCO1 ; Nc1ncccc1-c1nc2ccc(-c3ccccc3)nc2n1-c1ccc(C(=O)NCc2ccc(C=O)c(O)c2)cc1]

### Step 1: Synthesis of 4-(2-(2-aminopyridin-3-yl)-5-phenyl-3H-imidazo[4,5-b]pyridin-3-yl)-N-(3-((tert-butyldimethylsilyl)oxy)-4-(1,3-dioxolan-2-yl)benzyl)benzamide

4-[2-(2-Aminopyridin-3-yl)-5-phenylimidazo[4,5-b]pyridin-3-yl]benzoic acid (Intermediate 29-2) (100 mg, 0.245 mmol, 1 equiv) and N,N-dimethylaminopyridine (0.28 g, 2.3 mmol, 5 equiv) were added to a solution of 1-{3-[(tert-butyldimethylsilyl)oxy]-4-(1,3-dioxolan-2-yl)phenyl}methanamine (Intermediate 137-1) (91 mg, 0.29 mmol, 1.2 equiv), N,N-diisopropylethylamine (159 mg, 1.23 mmol, 5 equiv) and HATU (187 mg, 0.490 mmol, 2 equiv) in N,N-dimethylformamide (2 mL). The resulting mixture was stirred at room temperature for 1 h. The reaction was quenched with water (10 mL) and extracted with ethyl acetate (10 mL×3). The combined extracts were washed with water and brine, dried over sodium sulfate, filtered, and concentrated in vacuo. The resulting residue was purified by flash column chromatography on silica gel using a 0-100% gradient of ethyl acetate in petroleum ether to afford 4-[2-(2-aminopyridin-3-yl)-5-phenylimidazo[4,5-b]pyridin-3-yl]-N-({3-[(tert-butyldimethylsilyl)oxy]-4-(1,3-dioxolan-2-yl)phenyl}methyl)benzamide (80 mg, 47%) as a white solid. MS (ESI) calculated for C₄₀H₄₂N₆O₄Si: 698.30 m/z, found 699.20 [M+H]⁺.

### Step 2: Synthesis of 4-(2-(2-aminopyridin-3-yl)-5-phenyl-3H-imidazo[4,5-b]pyridin-3-yl)-N-(4-formyl-3-hydroxybenzyl)benzamide (Example 137)

4-(2-(2-aminopyridin-3-yl)-5-phenyl-3H-imidazo[4,5-b]pyridin-3-yl)-N-(3-((tert-butyldimethylsilyl)oxy)-4-(1,3-dioxolan-2-yl)benzyl)benzamide (80 mg, 0.11 mmol, 1 equiv) was dissolved in dichloromethane (5 mL) and 2,2,2-trifluoroacetic acid (1 mL). The reaction mixture was stirred at room temperature for 1 h. The resulting mixture was concentrated in vacuo and purified by preparative HPLC on a YMC-Actus Triart C18 ExRS column using a 17-42% gradient of acetonitrile in water (+10 mmol/L ammonium bicarbonate) to afford 4-[2-(2-aminopyridin-3-yl)-5-phenylimidazo[4,5-b]pyridin-3-yl]-N-[(4-formyl-3-hydroxyphenyl)methyl]benzamide (Example 137) (8.7 mg, 14%) as a yellow solid. MS (ESI) calculated for C₃₂H₂₄N₆O₃: 540.19 m/z, found 541.20 [M+H]⁺. ¹H NMR (300 MHz, DMSO-d₆) δ (ppm): 10.02 (s, 1H), 8.23 (d, J=8.4 Hz, 1H), 8.12-7.95 (m, 6H), 7.75-7.61 (m, 3H), 7.51-7.36 (m, 4H), 7.07 (d, J=8.3 Hz, 1H), 6.99 (s, 1H), 6.56 (in, J=7.7, 5.0 Hz, 1H), 4.64 (s, 2H).

### Intermediate 137-1: (3-((tert-butyldimethylsilyl)oxy)-4-(1,3-dioxolan-2-yl)phenyl) methanamine

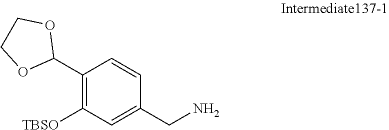 [SMILES: CC(C)(C)[Si](C)(C)Oc1cc(CN)ccc1C1OCCO1]

### Synthetic Route:

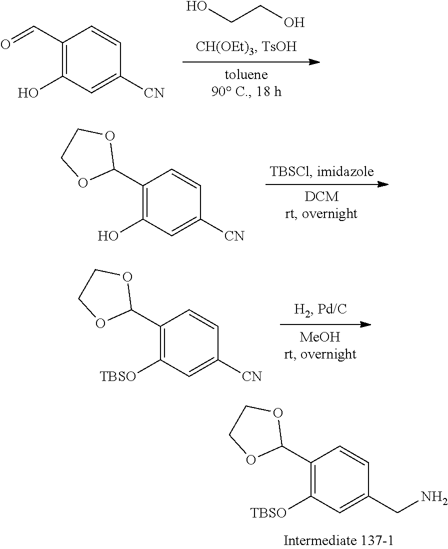

### Step 1: Synthesis of 4-bromo-2-(1,3-dioxolan-2-yl)phenol

To a solution of 4-formyl-3-hydroxybenzonitrile (7.31 g, 49.7 mmol, 1 equiv) in toluene (100 mL) was added p-toluenesulfonic acid (0.86 g, 5.0 mmol, 0.1 equiv), ethylene glycol (15.44 g, 248.7 mmol, 5 equiv) and triethyl orthoformate (22.12 g, 149.2 mmol, 3 equiv). The resulting solution was stirred at room temperature for 10 min then at 90° C. for 18 h. The resulting mixture was cooled to 0° C. and quenched by the addition of saturated aqueous ammonium chloride (80 mL) The resulting mixture was extracted with ethyl acetate (100 mL×3). The combined organic layers were washed with water (100 mL×3), dried over anhydrous sodium sulfate, filtered, and concentrated under reduced pressure. The residue was purified by silica gel column chromatography using a 0-7% gradient of ethyl acetate in petroleum ether to afford 4-(1,3-dioxolan-2-yl)-3-hydroxybenzonitrile (5.0 g, 53%) as a light-yellow oil. MS (ESI) calculated for C₁₀H₉NO₃: 191.06 m/z, found 192.10 [M+H]⁺.

### Step 2: Synthesis of 3-((tert-butyldimethylsilyl)oxy)-4-(1,3-dioxolan-2-yl)benzonitrile

To a solution of 4-(1,3-dioxolan-2-yl)-3-hydroxybenzonitrile (1.48 g, 7.75 mmol, 1 equiv) and imidazole (1.05 g, 15.5 mmol, 2 equiv) in dichloromethane (200 mL) was added tert-butyldimethylsilyl chloride (1.64 g, 10.9 mmol, 1.4 equiv). After stirring overnight at room temperature, the reaction was quenched by the addition of water (200 mL) at 0° C. The resulting mixture was extracted with ethyl acetate (300 mL×3). The combined organic layers were washed with water (300 mL×3), dried over anhydrous sodium sulfate, filtered, and concentrated under reduced pressure. The residue was purified by silica gel column chromatography using a 0-7% gradient of ethyl acetate in petroleum ether to afford 3-((tert-butyldimethylsilyl)oxy)-4-(1,3-dioxolan-2-yl)benzonitrile (1.8 g, 79%) as a colorless oil. MS (ESI) calculated for C₁₆H₂₃NO₃Si: 305.14 m/z, found 306.15 [M+H]⁺.

### Step 3: Synthesis of (3-((tert-butyldimethylsilyl)oxy)-4-(1,3-dioxolan-2-yl)phenyl) methanamine (Intermediate 137-1)

A solution of 3-[(tert-butyldimethylsilyl)oxy]-4-(1,3-dioxolan-2-yl)benzonitrile (200 mg, 0.655 mmol, 1 equiv) in methanol (10 mL) was treated with 10% palladium on carbon (15 mg, 0.14 mmol, 0.46 equiv) and the resulting mixture was stirred at room temperature under hydrogen atmosphere overnight. The resulting mixture was filtered, rinsing with methanol (20 mL×5). The filtrate was concentrated under reduced pressure to afford (3-((tert-butyldimethylsilyl)oxy)-4-(1,3-dioxolan-2-yl)phenyl)methanamine (Intermediate 137-1) (160 mg, 79%) as yellow oil, which was used in subsequent transformations without further purification. MS (ESI) calculated for C₁H₂₇NO₃Si: 309.18 m/z, found 310.20 [M+H]⁺.

### Example 138: N-(4-(2-(2-aminopyridin-3-yl)-5-phenyl-3H-imidazo[4,5-b]pyridin-3-yl)benzyl)-4-formyl-3-hydroxybenzamide

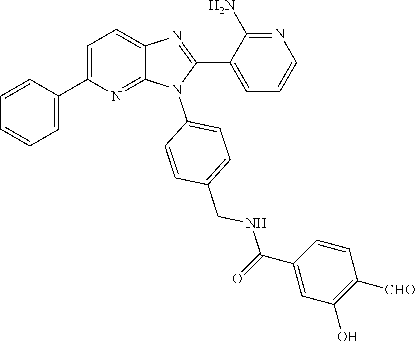 [SMILES: Nc1ncccc1-c1nc2ccc(-c3ccccc3)nc2n1-c1ccc(CNC(=O)c2ccc(C=O)c(O)c2)cc1]

### Synthetic Route:

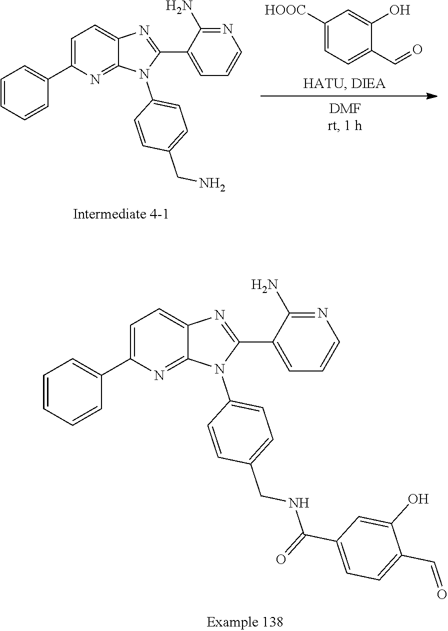 [SMILES: NCc1ccc(-n2c(-c3cccnc3N)nc3ccc(-c4ccccc4)nc32)cc1 ; O=Cc1ccc(C(=O)O)cc1O ; Nc1ncccc1-c1nc2ccc(-c3ccccc3)nc2n1-c1ccc(CNC(=O)c2ccc(C=O)c(O)c2)cc1]

### Step 1: Synthesis of N-({4-[2-(2-aminopyridin-3-yl)-5-phenylimidazo[4,5-b]pyridin-3-yl]phenyl}methyl)-4-formyl-3-hydroxybenzamide (Example 138)

A mixture of 3-{3-[4-(Aminomethyl)phenyl]-5-phenylimidazo[4,5-b]pyridin-2-yl}pyridin-2-amine (Intermediate 4-1) (170 mg, 0.433 mmol, 1 equiv), 4-formyl-3-hydroxybenzoic acid (108 mg, 0.650 mmol, 1.5 equiv), HATU (247 mg, 0.650 mmol, 1.5 equiv) and N,N-diisopropylethylamine (0.4 mL, 2.3 mmol, 5.3 equiv) in N,N-dimethylformamide (5 mL) was stirred at room temperature for 1 h. The reaction was quenched with water (5 mL). The resulting mixture was extracted with ethyl acetate (3×50 mL). The organic layers were combined, dried over anhydrous sodium sulfate, filtered, and concentrated in vacuo. The residue obtained was purified by preparative HPLC on a XBridge Shield RP18 OBD Column using a 33-58% gradient of acetonitrile in water (+10 mmol/L ammonium bicarbonate) to afford N-({4-[2-(2-aminopyridin-3-yl)-5-phenylimidazo[4,5-b]pyridin-3-yl]phenyl}methyl)-4-formyl-3-hydroxybenzamide (Example 138) (5.1 mg, 2.2%) as a yellow solid. MS (ESI) calculated for C₃₂H₂₄N₆O₃: 540.19 m/z, found 541.15 [M+H]⁺. ¹H NMR (300 MHz, DMSO-d₆) δ (ppm) 10.31 (s, 1H), 9.30-9.23 (m, 1H), 8.28-8.22 (m, 1H), 8.06-7.92 (m, 4H), 7.74-7.70 (m, 1H), 7.51-7.49 (m, 1H), 7.49-7.37 (m, 8H), 7.42-7.27 (m, 1H), 7.23-7.17 (m, 1H), 6.93 (s, 2H), 6.43-6.38 (m, 1H), 4.61-4.56 (m, 2H).

### Example 139: N-(1-{4-[2-(2-aminopyridin-3-yl)-5-methylimidazo[4,5-b]pyridin-3-yl]phenyl}cyclopropyl)-2-(2-fluoro-4-formyl-3-hydroxyphenyl)acetamide

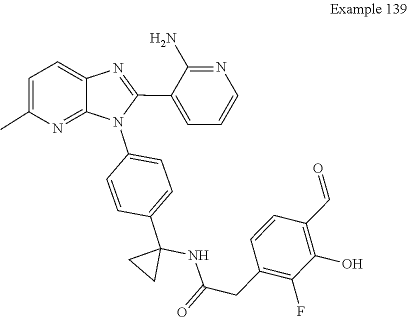 [SMILES: Cc1ccc2nc(-c3cccnc3N)n(-c3ccc(C4(NC(=O)Cc5ccc(C=O)c(O)c5F)CC4)cc3)c2n1]

Example 139 was prepared in a manner analogous to Example 1 (starting from Step 3) using Intermediate 139-1 in place of the amine starting material and Intermediate 21-2 in place of Intermediate 1-4. MS (ESI) calculated for C₃₀H₂₅FN₆O₃: 536.20 m/z, found 537.20 [M+H]⁺. ¹H-NMR (400 MHz, DMSO-d₆) δ (ppm): 10.19 (s, 1H), 8.99 (s, 1H), 8.11-8.13 (m, 1H), 8.01-8.03 (m, 1H), 7.67-7.70 (m, 1H), 7.43-7.46 (m, 1H), 7.30-7.36 (m, 3H), 7.22-7.24 (m, 2H), 6.91-6.95 (m, 1H), 6.73-6.77 (m, 1H), 3.64 (s, 2H), 2.50-2.52 (m, 3H), 1.21-1.30 (m, 4H). ¹⁹F-NMR (400 MHz, DMSO-d₆) δ (ppm): −139.30.

### Intermediate 139-1: 3-(3-(4-(1-aminocyclopropyl)phenyl)-5-methyl-3H-imidazo[4,5-b]pyridin-2-yl)pyridin-2-amine

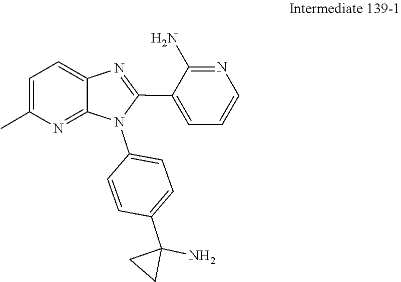 [SMILES: Cc1ccc2nc(-c3cccnc3N)n(-c3ccc(C4(N)CC4)cc3)c2n1]

Intermediate 139-1 was prepared in a manner analogous to Intermediate 10-1 (starting from Step 2) using 6-methyl-3-nitropyridin-2-amine in place of the amine starting material and tert-butyl N-[1-(4-bromophenyl)cyclopropyl]carbamate in place of tert-butyl N-[(4-bromophenyl)methyl]carbamate. MS (ESI) calculated for C₂₁H₂₀N₆: 356.17 m/z, found 357.15 [M+H]⁺.

### Example 140: N-(1-{4-[2-(2-aminopyridin-3-yl)-5-(morpholin-4-yl)imidazo[4,5-b]pyridin-3-yl]phenyl}cyclopropyl)-2-(2-fluoro-4-formyl-3-hydroxyphenyl)acetamide

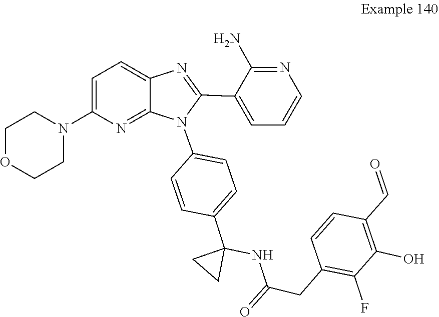 [SMILES: Nc1ncccc1-c1nc2ccc(N3CCOCC3)nc2n1-c1ccc(C2(NC(=O)Cc3ccc(C=O)c(O)c3F)CC2)cc1]

Example 140 was prepared in a manner analogous to Example 1 (starting from Step 3) using Intermediate 140-2 in place of the amine starting material and Intermediate 21-1 in place of Intermediate 1-4. MS (ESI) calculated for C₃₃H₃₀FN₇O₄: 607.23 m/z, found 608.20 [M+H]⁺. ¹H-NMR (400 MHz, DMSO-d₆) δ (ppm): 10.19 (s, 1H), 7.91-8.09 (m, 2H), 7.39-7.59 (m, 2H), 7.29-7.38 (m, 2H), 7.15-7.21 (m, 2H), 6.85-6.99 (m, 2H), 6.70-6.79 (m, 1H), 3.65-3.76 (m, 6H), 3.30-3.42 (m, 4H), 1.15-1.35 (m, 4H). ¹⁹F-NMR (400 MHz, DMSO-d₆) δ (ppm): −139.29.

### Intermediate 140-2: 3-(3-(4-(1-aminocyclopropyl)phenyl)-5-morpholino-3H-imidazo[4,5-b]pyridin-2-yl)pyridin-2-amine

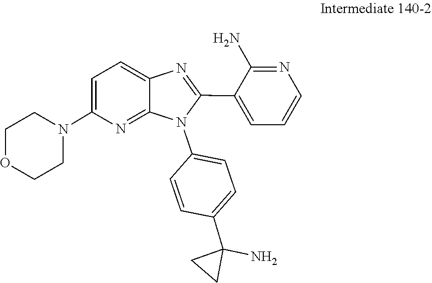 [SMILES: Nc1ncccc1-c1nc2ccc(N3CCOCC3)nc2n1-c1ccc(C2(N)CC2)cc1]

Intermediate 140-2 was prepared in a manner analogous to Intermediate 10-1 (starting from Step 2) using Intermediate 140-1 in place of the amine starting material and tert-butyl N-[1-(4-bromophenyl)cyclopropyl]carbamate in place of tert-butyl N-[(4-bromophenyl)methyl]carbamate. MS (ESI) calculated for C₂₄H₂₅N₇O: 427.21 m/z, found 428.20 [M+H]⁺.

### Intermediate 140-1: 6-morpholino-3-nitropyridin-2-amine

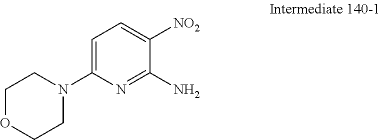 [SMILES: Nc1nc(N2CCOCC2)ccc1[N+](=O)[O-]]

### Synthetic Route:

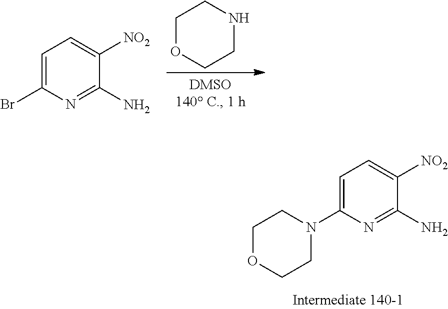 [SMILES: Nc1nc(Br)ccc1[N+](=O)[O-] ; C1COCCN1 ; Nc1nc(N2CCOCC2)ccc1[N+](=O)[O-]]

### Step 1: Synthesis of 6-(morpholin-4-yl)-3-nitropyridin-2-amine (Intermediate 140-1)

To a solution of 6-bromo-3-nitropyridin-2-amine (2.0 g, 9.2 mmol, 1 equiv) in dimethyl sulfoxide (16 mL) was added morpholine (7.99 g, 91.6 mmol, 10 equiv). The resulting mixture was stirred at 140° C. for 1 h. The mixture was concentrated in vacuo to afford 6-(morpholin-4-yl)-3-nitropyridin-2-amine (Intermediate 140-1) (1.92 g, 93%) as a yellow solid, which was used without purification in subsequent transformations. MS (ESI) calculated for C₉H₁₂N₄O₃: 224.09 m/z, found 225.05 [M+H]⁺.

### Example 141: N-(1-{4-[2-(2-aminopyridin-3-yl)-5-(pyrazol-1-yl)imidazo[4,5-b]pyridin-3-yl]phenyl}cyclopropyl)-2-(2-fluoro-4-formyl-3-hydroxyphenyl)acetamide

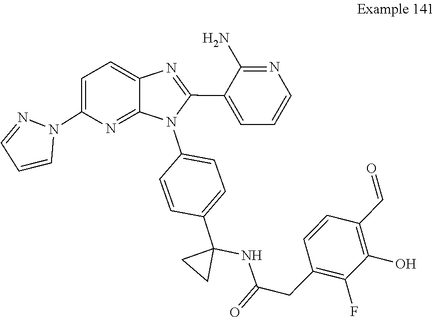 [SMILES: Nc1ncccc1-c1nc2ccc(-n3cccn3)nc2n1-c1ccc(C2(NC(=O)Cc3ccc(C=O)c(O)c3F)CC2)cc1]

Example 141 was prepared in a manner analogous to Example 1 (starting from Step 3) using Intermediate 141-2 in place of the amine starting material and Intermediate 21-1 in place of Intermediate 1-4. MS (ESI) calculated for C₃₂H₂₅FN₈03: 588.20 m/z, found 589.20 [M+H]⁺. ¹H NMR (400 MHz, DMSO-d₆) δ (ppm): 10.12 (s, 1H), 8.38-8.46 (m, 1H), 8.29-8.32 (m, 1H), 7.91-8.05 (m, 2H), 7.75-7.87 (m, 2H), 7.39-7.51 (m, 3H), 7.19-7.23 (m, 2H), 6.88-6.92 (m, 1H), 6.79-6.82 (m, 1H), 6.47-6.53 (m, 1H), 3.55-3.63 (m, 2H), 1.14-1.33 (m, 4H).

### Intermediate 141-2: 3-(3-(4-(1-aminocyclopropyl)phenyl)-5-(1H-pyrazol-1-yl)-3H-imidazo[4,5-b]pyridin-2-yl)pyridin-2-amine

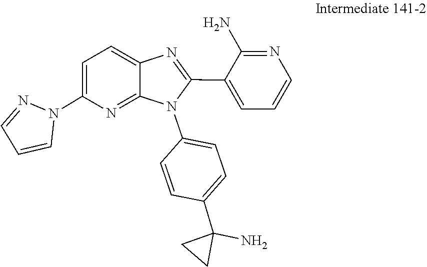 [SMILES: Nc1ncccc1-c1nc2ccc(-n3cccn3)nc2n1-c1ccc(C2(N)CC2)cc1]

Intermediate 141-2 was prepared in a manner analogous to Intermediate 10-1 (starting from Step 2) using Intermediate 141-1 in place of the amine starting material and tert-butyl N-[1-(4-bromophenyl)cyclopropyl]carbamate in place of tert-butyl N-[(4-bromophenyl)methyl]carbamate. MS (ESI) calculated for C₂₃H₂₀N₈: 408.18 m/z, found 409.20 [M+H]⁺.

### Intermediate 141-1: 3-nitro-6-(1H-pyrazol-1-yl)pyridin-2-amine

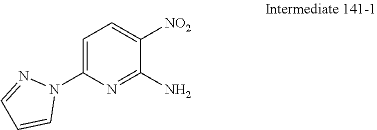 [SMILES: Nc1nc(-n2cccn2)ccc1[N+](=O)[O-]]

### Synthetic Route:

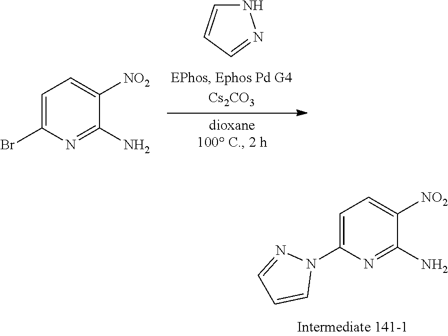 [SMILES: c1cn[nH]c1 ; Nc1nc(Br)ccc1[N+](=O)[O-] ; Nc1nc(-n2cccn2)ccc1[N+](=O)[O-]]

### Step 1: Synthesis of 3-nitro-6-(1H-pyrazol-1-yl)pyridin-2-amine (Intermediate 141-1)

A suspension of 6-bromo-3-nitropyridin-2-amine (2.0 g, 9.2 mmol), pyrazole (937 mg, 13.8 mmol), EPhos (736 mg, 1.38 mmol), EPhos Pd G4 (843 mg, 0.918 mmol) and cesium carbonate (6.0 g, 18 mmol) in 1,4-dioxane (20 mL) was stirred at 100° C. for 2 h under nitrogen atmosphere. The resulting mixture was cooled to room temperature and diluted with water (100 mL). The resulting mixture was extracted with ethyl acetate (100 mL×3). The combined extracts were concentrated under vacuum and the resulting oil was purified by silica gel column chromatography eluting with dichloromethane to afford 3-nitro-6-(pyrazol-1-yl)pyridin-2-amine (Intermediate 141-1) as brown solid (1.72 g, 91%). MS (ESI) calculated for C₈H₇N₅O₂: 205.06 m/z, found 206.05 [M+H]⁺.

### Example 142: Protein Expression and Purification

Unless specified otherwise, the following protein sequences and protein expression protocols were used for experimental data collection. Amino acid mutation numbers are based on the naturally occurring human AKT1 amino acid sequence and not the genetically modified sequence (e.g., the E17K mutation is shown as the 29^{th} residue in the following sequences).

AKT1 WT Amino Acid Sequence:
HHHHHHENLYFQGSDVAIVKEGWLHKRGEYIKTWRPRYFLLKNDGTFIG
YKERPQDVDQREAPLNNFSVAQCQLMKTERPRPNTFIIRCLQWTTVIER
TFHVETPEEREEWTTAIQTVADGLKKQEEEEMDFRSGSPSDNSGAEEME
VSLAKPKHRVTMNEFEYLKLLGKGTFGKVILVKEKATGRYYAMKILKKE
VIVAKDEVAHTLTENRVLQNSRHPFLTALKYSFQTHDRLCFVMEYANGG
ELFFHLSRERVFSEDRARFYGAEIVSALDYLHSEKNVVYRDLKLENLML
DKDGHIKITDFGLCKEGIKDGATMKTFCGTPEYLAPEVLEDNDYGRAVD
WWGLGVVMYEMMCGRLPFYNQDHEKLFELILMEEIRFPRTLGPEAKSLL
SGLLKKDPKQRLGGGSEDAKEIMQHRFFAGIVWQHVYEKKLSPPFKPQV
TSETDTRYFDEEFTAQMITITPPDQDDSMECVDSERRPHFPQFSYSASG
TA
AKT1 E17K Amino Acid Sequence:
HHHHHHENLYFQGSDVAIVKEGWLHKRGKYIKTWRPRYFLLKNDGTFIG
YKERPQDVDQREAPLNNFSVAQCQLMKTERPRPNTFIIRCLQWTTVIER
TFHVETPEEREEWTTAIQTVADGLKKQEEEEMDFRSGSPSDNSGAEEME
VSLAKPKHRVTMNEFEYLKLLGKGTFGKVILVKEKATGRYYAMKILKKE
VIVAKDEVAHTLTENRVLQNSRHPFLTALKYSFQTHDRLCFVMEYANGG
ELFFHLSRERVFSEDRARFYGAEIVSALDYLHSEKNVVYRDLKLENLML
DKDGHIKITDFGLCKEGIKDGATMKTFCGTPEYLAPEVLEDNDYGRAVD
WWGLGVVMYEMMCGRLPFYNQDHEKLFELILMEEIRFPRTLGPEAKSLL
SGLLKKDPKQRLGGGSEDAKEIMQHRFFAGIVWQHVYEKKLSPPFKPQV
TSETDTRYFDEEFTAQMITITPPDQDDSMECVDSERRPHFPQFSYSASG
TA

The human AKT1 (WT or E17K) with the N-terminal affinity 6-His-tag followed by a cleavable TEV-tag was expressed in Sf9 insect cells using baculovirus-insect cell expression system, which were infected in 2 million per milliliter at 300 K. The cells were harvested after an incubation time of 72 hours at 300 K and stored at 193 K until lysis.

The cell paste from 1 L Sf9 cell pellet were lysed in (50 mM Tris, 500 mM NaCl, 1 mM TCEP, 10% glycerol, 0.1% Triton X-100, pH 8.0) supplemented with cOmplete EDTA-free protease inhibitor tablet (Roche). Purification was performed by Ni-NTA capture of the His-tagged protein in buffer A, washed with (50 mM Tris, 500 mM NaCl, 1 mM TCEP, 10% glycerol, pH 8.0, 20 mM imidazole), and eluted in two steps with (50 mM Tris, 500 mM NaCl, 1 mM TCEP, 10% glycerol, pH 8.0, 50 and 250 mM imidazole). Fraction eluted with 50 mM imidazole was dialyzed against 4 L dialysis buffer and digested by His-tagged TEV (TEV:Protein=1:15) at 4° C. overnight and then loaded onto 2nd Ni-NTA column. The flow through were pooled and diluted to 50 mM NaCl, and then load onto Superdex 75 100/300 GL column, separately using SEC buffer (25 mM Tris, 100 mM NaCl, 5 mM DTT, 10% glycerol, pH 7.5). The tag cleaved AKT1 (2-480, WT or E17K) were concentrated in final storage buffer (25 mM Tris, 100 mM NaCl, 5 mM DTT, 10% glycerol, pH 7.5) and stored in −80° C.

### Example 143: Intact Mass Spectrometry and Competition Studies

### Identification of Covalent Binding by Intact Mass Spectrometry for Compounds

AKT1 WT or AKT1 E17K (1 μM) was treated with a compound from Table 1 (1.5 μM) for 2 hrs at 37° C., with and without ARQ092 (50 μM) competitor as a specificity control. Reversible imines were captured by reductive amination with 5 mM NaBH4 for 5 min. Reduction reactions were quenched by diluting the sample in equal volume acetonitrile+1% TFA, then analyzed by LCMS.

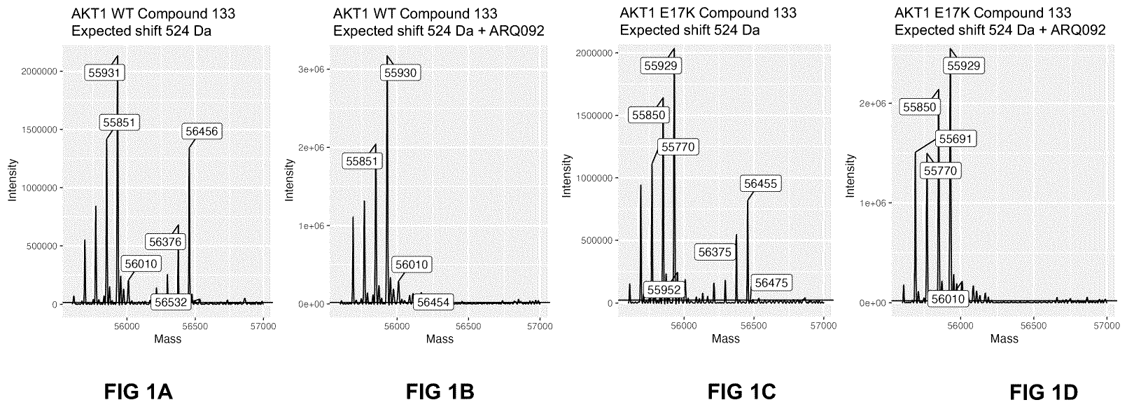

FIGS. 1 to 25 provide intact mass spectra for AKT1 WT protein and AKT1 E17K mutant protein after incubation with compounds of the present disclosure and subsequent borohydride reduction. Sodium borohydride was added to reduce the reversible imine bond between the AKT1 protein and compounds of the present disclosure, thereby forming a more stable C—N single bond between the protein and the compound and allowing detection of a covalent complex by intact mass spec. Further, FIGS. 1 to 10, 12, and 14 to 25 each provide intact mass spectra following the incubation of an AKT1 protein with a compound of the present disclosure both in the absence of a competitor molecule (i.e., ARQ-092, a molecule known to bind to and inhibit the function of AKT1); and in the presence of a significant stoichiometric excess of a competitor molecule.

FIG. 1 provides intact mass spectra following incubation of Compound 133 with AKT1 WT protein both in the absence of any competitor molecule (FIG. 1A) and in the presence of a significant molar excess of competitor molecule ARQ-092 (FIG. 1B); and intact mass spectra following incubation of Compound 133 with AKT1 E17K mutant protein both in the absence of any competitor molecule (FIG. 1C) and in the presence of a significant molar excess of competitor molecule ARQ-092 (FIG. 1D). As shown in FIG. 1A, incubation of Compound 133 with AKT1 WT protein and AKT1 E17K mutant protein resulted in covalent complex formation between Compound 133 and the AKT1 protein. The data provided demonstrates two different sets of m/z peaks, which correspond to the mass of the unmodified AKT1 WT protein and the covalent complex between Compound 133 and AKT1 WT protein. The first set of peaks on the left side of the chart (between mass values of 55500 and 56000 Da, and including specific observed mass values of 55851 and 55931 Da) corresponds to the unmodified AKT1 WT protein, with the different individual peaks corresponding to different phosphorylation states of the protein. The second set of peaks to the right of the first set (between mass values of 56100 and 56500 Da, and including specific observed mass values of 56376 and 56456 Da) corresponds to the covalent complex formed between Compound 133 and AKT1 WT protein, with the different individual peaks corresponding to different phosphorylation states of the protein-compound complex. Thus, as seen in FIG. 1A a noticeable level of covalent complex was formed between Compound 133 and the AKT1 protein, indicated by relative intensity of the peaks corresponding to the covalent construct compared to those corresponding to the unmodified protein. Furthermore, the extent of covalent complex formation was likely to be even higher than shown in FIG. 1A, as some of the peak intensity for the peaks corresponding to unmodified protein was attributable to fragmentation of the C—N bond between Compound 133 and the AKT1 protein (formed by borohydride reduction of the imine bond in the covalent complex) during ionization and detection. In contrast, FIG. 1B shows essentially no covalent complex formation when AKT1 WT protein was incubated with Compound 133 in the presence of a significant stoichiometric excess of a competitor molecule ARQ-092, and instead only mass peaks corresponding to the unmodified protein (set of peaks between mass values of 55500 and 56000 Da, and including specific observed mass values of 55851 and 55930 Da). Thus, the data provided in FIGS. 1A and 1B show that (1) Compound 133 formed a covalent complex with AKT1 WT protein, and (2) Compound 133 bound to the same binding pocket of AKT1 as the competitor molecule, as shown by the significant reduction in observed complex formation in the presence of a stoichiometric excess of competitor molecule (compare FIG. 1B to FIG. 1A). As seen in FIG. 1C, a covalent complex between Compound 133 and AKT1 E17K mutant protein was observed (set of peaks between mass values of 56100 and 56500 Da, and including specific observed mass values of 56375 and 56455 Da), with unmodified AKT1 E17K mutant protein (or fragmentation of the covalent complex), indicated by the set of peaks between mass values of 55500 and 56000 Da, including specific observed mass values of 55770, 55850, and 55929 Da. In contrast, FIG. 1D shows essentially no covalent complex formation when AKT1 E17K was incubated with Compound 133 in the presence of a significant stoichiometric excess of a competitor molecule ARQ-092, and instead only mass peaks corresponding to the unmodified protein (set of peaks between mass values of 55500 and 56000 Da, and including specific observed mass values of 55691, 55770, 55850, and 55929 Da. Thus, the data provided in FIGS. 1C and 1D show that (1) Compound 133 formed a covalent complex with AKT1 E17K, and (2) Compound 133 bound to the same binding pocket of AKT1 E17K as the competitor molecule, as shown by the significant reduction in observed complex formation in the presence of a stoichiometric excess of competitor molecule (compare FIG. 1D to FIG. 1C).

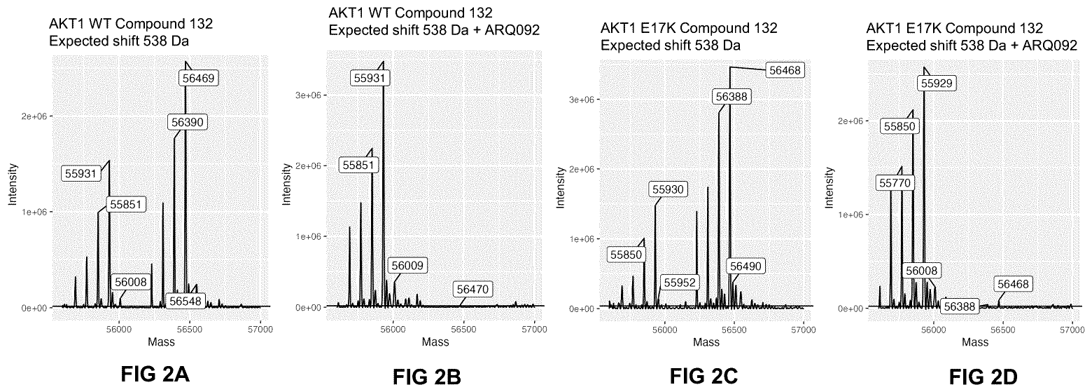

FIG. 2 provides intact mass spectra following incubation of Compound 132 with AKT1 WT protein or AKT1 E17K mutant protein, both in the absence of any competitor molecule (FIGS. 2A, 2C) and in the presence of a significant molar excess of competitor molecule ARQ-092 (FIGS. 2B, 2D). FIG. 2A shows the presence of covalent complex with AKT1 WT protein (peaks corresponding to masses of 56390 and 56469 Da), and some unmodified protein (peaks corresponding to masses of 55851 and 55931 Da). Essentially no covalent complex formation was observed in FIG. 2B, indicating that Compound 132 bound to the same AKT1 WT binding pocket as the competitor molecule. FIG. 2C shows the presence of covalent complex with AKT1 E17K mutant protein (peaks corresponding to masses of 56388 and 56468 Da), and some unmodified mutant protein (peaks corresponding to masses of 55850 and 55930 Da). Essentially no covalent complex formation was observed in FIG. 2D, indicating that Compound 132 bound to the same AKT1 E17K binding pocket as the competitor molecule.

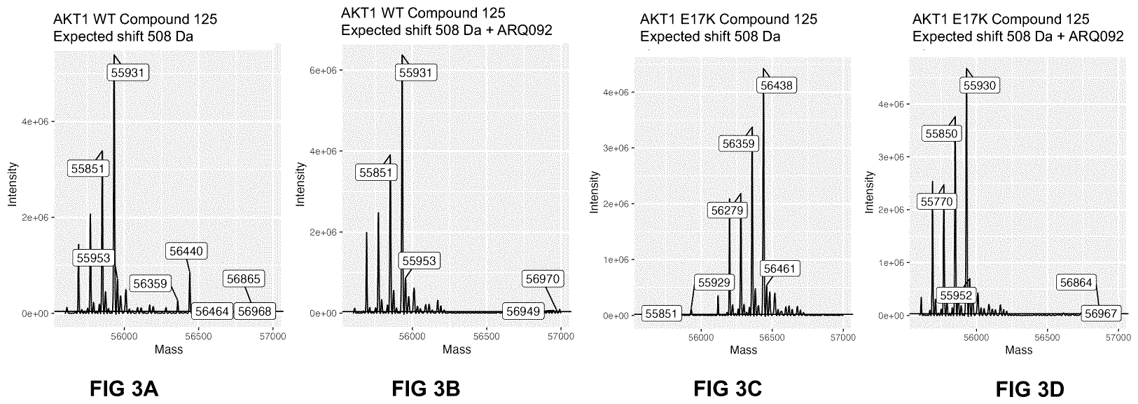

FIG. 3 provides intact mass spectra following incubation of Compound 125 with AKT1 WT protein or AKT1 E17K mutant protein, both in the absence of any competitor molecule (FIGS. 3A, 3C) and in the presence of a significant molar excess of competitor molecule ARQ-092 (FIGS. 3B, 3D). For the AKT1 WT protein ender both conditions, only peaks corresponding to unmodified WT protein (peaks corresponding to masses 55953, 55851, and 55931 Da) were observed for Compound 125, indicating that either little to no covalent complex with AKT1 WT was formed, or the resulting C—N bond completely fragmented during ionization and/or detection. FIG. 3C shows the presence of covalent complex of Compound 125 with AKT1 E17K mutant protein (peaks corresponding to masses of 56279, 56359, and 56438 Da), and essentially no unmodified mutant protein. Essentially no covalent complex formation was observed in FIG. 3D, indicating that Compound 125 bound to the same AKT1 E17K binding pocket as the competitor molecule.

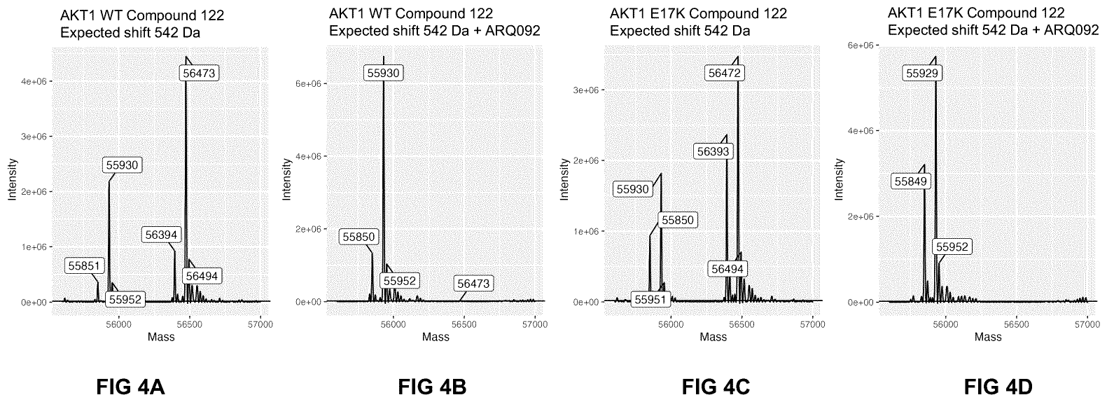

FIG. 4 provides intact mass spectra following incubation of Compound 122 with AKT1 WT protein or AKT1 E17K mutant protein, both in the absence of any competitor molecule (FIGS. 4A, 4C) and in the presence of a significant molar excess of competitor molecule ARQ-092 (FIGS. 4B, 4D). FIG. 4A shows the presence of covalent complex of Compound 122 with AKT1 WT protein (peaks corresponding to masses of 56394 and 56473 Da), and some unmodified protein (peaks corresponding to masses of 55851 and 55930 Da). Essentially no covalent complex formation was observed in FIG. 4B, indicating that Compound 122 bound to the same AKT1 WT binding pocket as the competitor molecule. FIG. 4C shows the presence of covalent complex with AKT1 E17K mutant protein (peaks corresponding to masses of 56393 and 56472 Da), and some unmodified mutant protein (peaks corresponding to masses of 55850 and 55930 Da). Essentially no covalent complex formation was observed in FIG. 4D, indicating that Compound 122 bound to the same AKT1 E17K binding pocket as the competitor molecule.

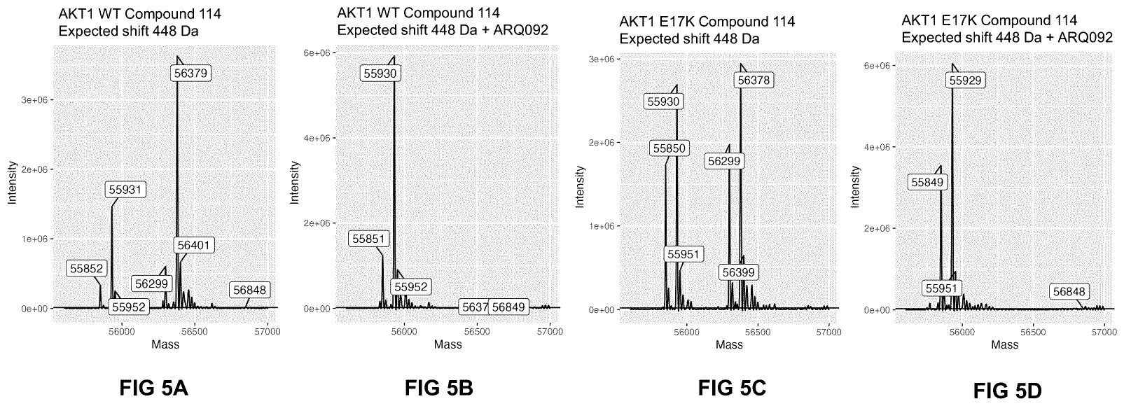

FIG. 5 provides intact mass spectra following incubation of Compound 114 with AKT1 WT protein or AKT1 E17K mutant protein, both in the absence of any competitor molecule (FIGS. 5A, 5C) and in the presence of a significant molar excess of competitor molecule ARQ-092 (FIGS. 5B, 5D). FIG. 5A shows the presence of covalent complex of Compound 114 with AKT1 WT protein (peaks corresponding to masses of 56299 and 56379 Da), and some unmodified protein (peaks corresponding to masses of 55852 and 55931 Da). Essentially no covalent complex formation was observed in FIG. 5B, indicating that Compound 114 bound to the same AKT1 WT binding pocket as the competitor molecule. FIG. 5C shows the presence of covalent complex with AKT1 E17K mutant protein (peaks corresponding to masses of 56299 and 56378 Da), and unmodified mutant protein (peaks corresponding to masses of 55850 and 55930 Da). Essentially no covalent complex formation was observed in FIG. 5D, indicating that Compound 114 bound to the same AKT1 E17K binding pocket as the competitor molecule.

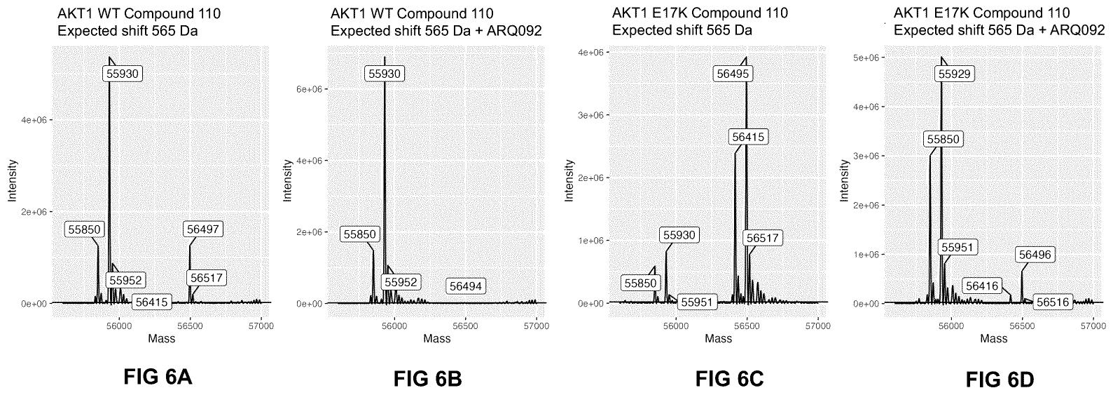

FIG. 6 provides intact mass spectra following incubation of Compound 110 with AKT1 WT protein or AKT1 E17K mutant protein, both in the absence of any competitor molecule (FIGS. 6A, 6C) and in the presence of a significant molar excess of competitor molecule ARQ-092 (FIGS. 6B, 6D). FIG. 6A shows a small presence of covalent complex of Compound 110 with AKT1 WT protein (peak corresponding to mass of 56497 Da), and some unmodified protein (peaks corresponding to masses of 55850 and 55930 Da). Essentially no covalent complex formation was observed in FIG. 6B, indicating that Compound 110 bound to the same AKT1 WT binding pocket as the competitor molecule. FIG. 6C shows significant presence of a covalent complex of Compound 110 with AKT1 ET7K mutant protein (peaks corresponding to masses of 56415 and 56495 Da), and a small level of unmodified mutant protein (peaks corresponding to masses of 55850 and 55930 Da). Essentially little to no covalent complex formation was observed in FIG. 6D, indicating that Compound 110 bound to the same AKT1 ET7K binding pocket as the competitor molecule. Comparing FIG. 6A to FIG. 6C shows that Compound 110 is selective for complex formation with AKT1 E17K.

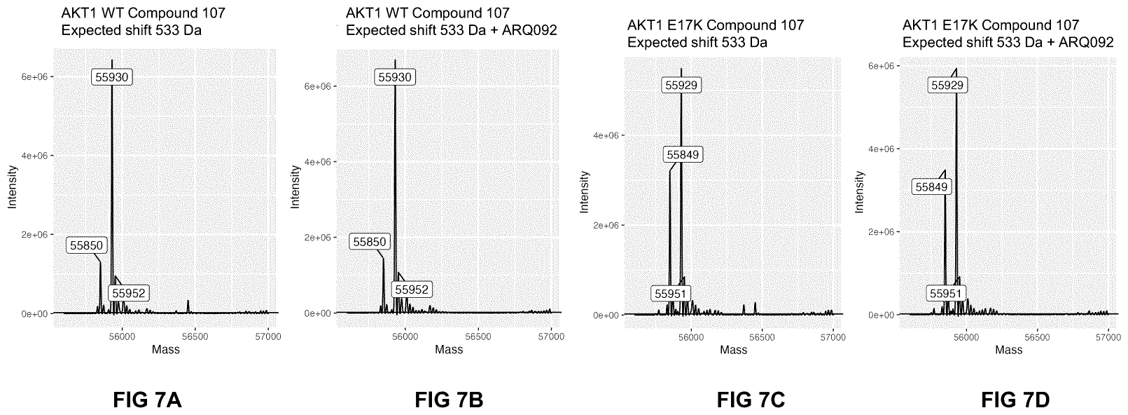

FIG. 7 provides intact mass spectra following incubation of Compound 107 with AKT1 WT protein or AKT1 E17K mutant protein, both in the absence of any competitor molecule (FIGS. 7A, 7C) and in the presence of a significant molar excess of competitor molecule ARQ-092 (FIGS. 7B, 7D). For the AKT1 WT protein under both conditions, FIG. 7A and 7B show only peaks corresponding to unmodified WT protein (55850 and 55930 Da) for Compound 107, indicating that either little to no covalent complex of Compound 107 with AKT1 WT was formed, or the resulting C—N bond completely fragmented during ionization and/or detection. For the AKT1 E17K mutant protein under both conditions, FIGS. 7C and 7D show only peaks corresponding to unmodified mutant protein (55849 and 55929 Da) for Compound 107, indicating that either little to no covalent complex of Compound 107 with AKT1 E17K was formed, or the resulting C—N bond completely fragmented during ionization and/or detection.

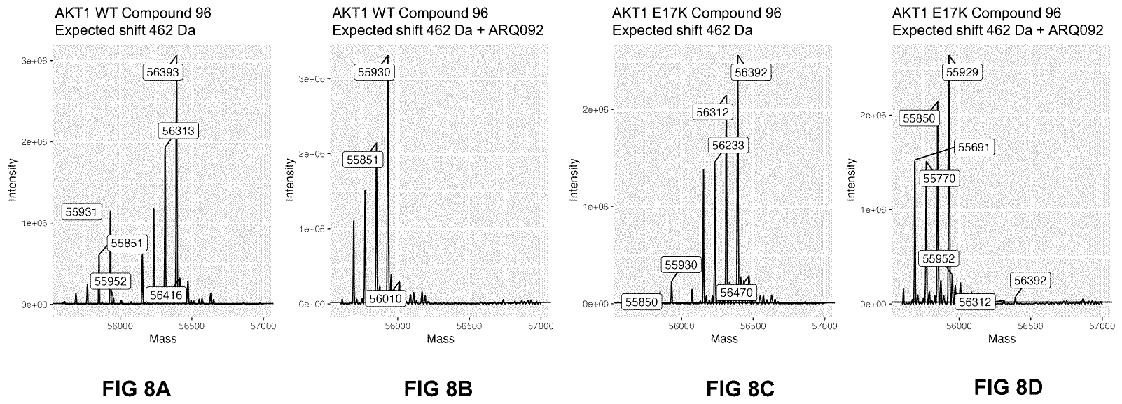

FIG. 8 provides intact mass spectra following incubation of Compound 96 with AKT1 WT protein or AKT1 E17K mutant protein, both in the absence of any competitor molecule (FIGS. 8A, 8C) and in the presence of a significant molar excess of competitor molecule ARQ-092 (FIGS. 8B, 8D). FIG. 8A shows a significant presence of covalent complex of Compound 96 with AKT1 WT protein (peaks corresponding to masses of 56303 and 56393 Da), and a small level of unmodified protein (peaks corresponding to masses of 55851 and 55931 Da). Essentially no covalent complex formation was observed in FIG. 8B, indicating that Compound 96 bound to the same AKT1 WT binding pocket as the competitor molecule. FIG. 8C shows significant presence of a covalent complex of Compound 96 with AKT1 E17K mutant protein (peaks corresponding to masses of 56233, 5656312, and 56392 Da), and almost no unmodified mutant protein (peaks corresponding to masses of 55850 and 55930 Da). Essentially little to no covalent complex formation was observed in FIG. 8D, indicating that 43 bound to the same AKT1 E17K binding pocket as the competitor molecule.

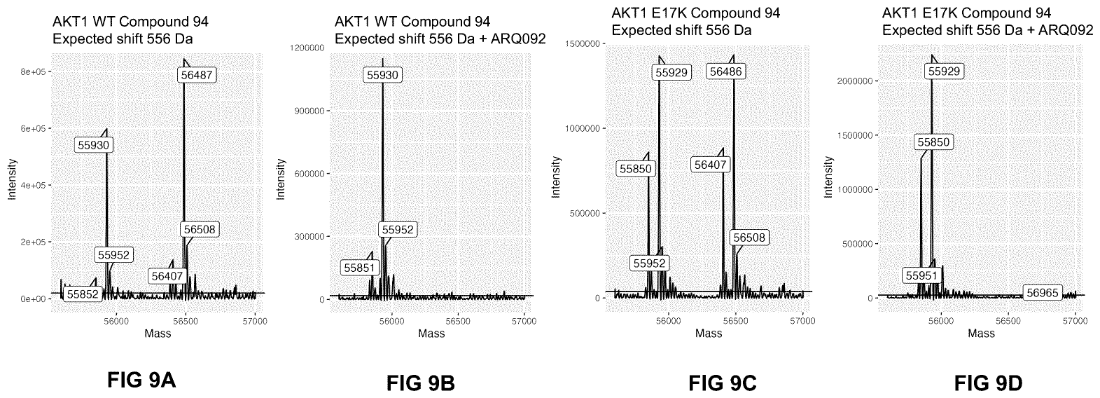

FIG. 9 provides intact mass spectra following incubation of Compound 94 with AKT1 WT protein or AKT1 E17K mutant protein, both in the absence of any competitor molecule (FIGS. 9A, 9C) and in the presence of a significant molar excess of competitor molecule ARQ-092 (FIGS. 9B, 9D). FIG. 9A shows the presence of a covalent complex of Compound 94 with AKT1 WT protein (peaks corresponding to masses of 56487 and 56508 Da), and some unmodified protein (peaks corresponding to masses of 55930 and 55952 Da). Essentially no covalent complex formation was observed in FIG. 9B, indicating that Compound 94 bound to the same AKT1 WT binding pocket as the competitor molecule. FIG. 9C shows the presence of a covalent complex of Compound 94 with AKT1 E17K mutant protein (peaks corresponding to masses of 56407 and 56486 Da), and unmodified mutant protein (peaks corresponding to masses of 55850 and 55929 Da). Essentially no covalent complex formation was observed in FIG. 9D, indicating that Compound 94 bound to the same AKT1 E17K binding pocket as the competitor molecule.

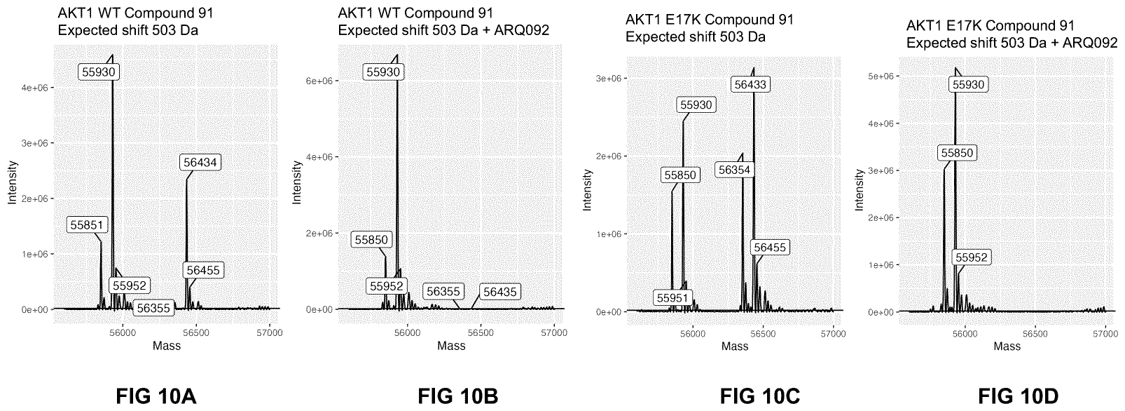

FIG. 10 provides intact mass spectra following incubation of Compound 91 with AKT1 WT protein or AKT1 E17K mutant protein, both in the absence of any competitor molecule (FIGS. 10A, 10C) and in the presence of a significant molar excess of competitor molecule ARQ-092 (FIGS. 10B, 10D). FIG. 10A shows the presence of a covalent complex of Compound 91 with AKT1 WT protein (peaks corresponding to masses of 56434 and 56455 Da), and some unmodified protein (peaks corresponding to masses of 55851 and 55930 Da). Essentially no covalent complex formation was observed in FIG. 10B, indicating that Compound 91 bound to the same AKT1 WT binding pocket as the competitor molecule. FIG. 10C shows the presence of a covalent complex of Compound 91 with AKT1 E17K mutant protein (peaks corresponding to masses of 56354 and 56433 Da), and unmodified mutant protein (peaks corresponding to masses of 55850 and 55930 Da). Essentially no covalent complex formation was observed in FIG. 10D, indicating that Compound 91 bound to the same AKT1 E17K binding pocket as the competitor molecule.

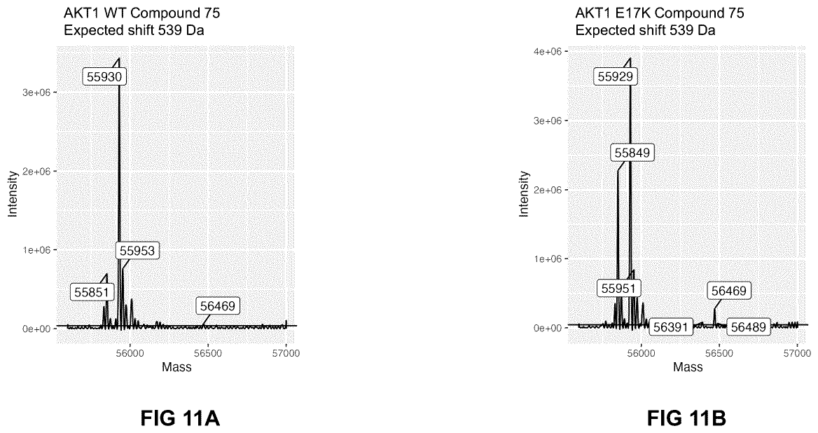

FIG. 11 provides intact mass spectra following incubation of Compound 75 with AKT1 WT protein (FIG. 11A) or AKT1 E17K mutant protein (FIG. 11B). For both the AKT1 WT protein and AKT1 E17K mutant protein, FIGS. 11A and 11B show only peaks corresponding to unmodified WT/mutant protein (55850/55849 and 55930/55929 Da) for Compound 75, indicating that either little to no covalent complex of Compound 75 with AKT1 WT or AKT1 E17K was formed, or the resulting C—N bond completely fragmented during ionization and/or detection.

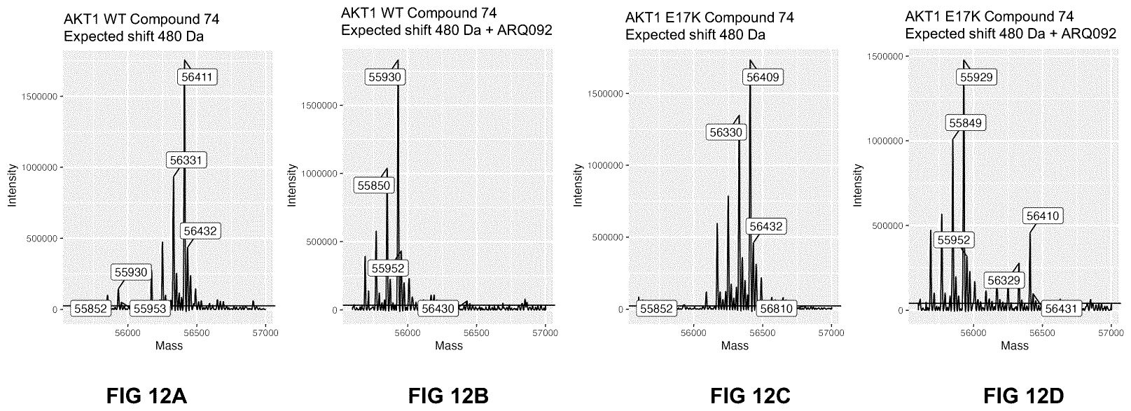

FIG. 12 provides intact mass spectra following incubation of Compound 74 with AKT1 WT protein or AKT1 E17K mutant protein, both in the absence of any competitor molecule (FIGS. 12A, 12C) and in the presence of a significant molar excess of competitor molecule ARQ-092 (FIGS. 12B, 12D). FIG. 12A shows a significant presence of covalent complex of Compound 74 with AKT1 WT protein (peaks corresponding to masses of 56331 and 56411 Da), and essentially no unmodified WT protein. Essentially no covalent complex formation was observed in FIG. 12B, indicating that Compound 74 bound to the same AKT1 WT binding pocket as the competitor molecule. FIG. 12C shows significant presence of a covalent complex of Compound 74 with AKT1 E17K mutant protein (peaks corresponding to masses of 56330 and 56409 Da), and essentially no unmodified mutant protein. Essentially little to no covalent complex formation was observed in FIG. 12D, indicating that Compound 74 bound to the same AKT1 E17K binding pocket as the competitor molecule.

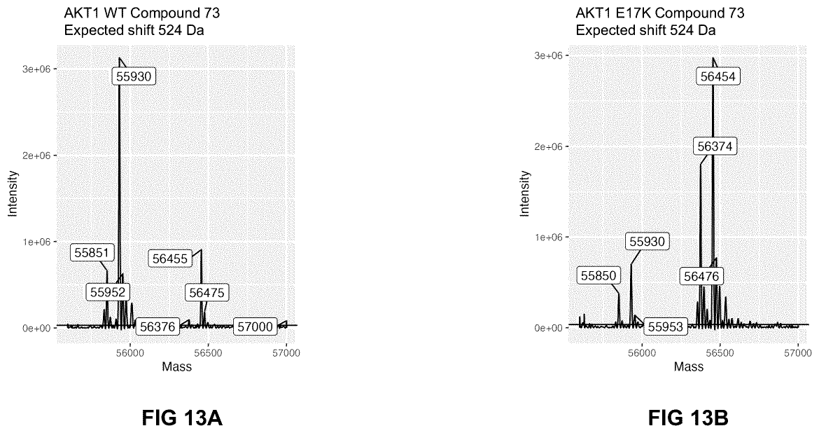

FIG. 13 provides intact mass spectra following incubation of Compound 73 with AKT1 WT protein (FIG. 13A) or AKT1 E17K mutant protein (FIG. 13B). For the AKT1 WT protein, FIG. 13A shows significant peaks corresponding to unmodified WT protein (55851 and 55930 Da) with a small level of a covalent complex of Compound 73 with AKT1 WT protein (peak corresponding to mass of 56455 Da), indicating that either a low level of covalent complex of Compound 73 with AKT1 WT was formed, or the resulting C—N bond mostly fragmented during ionization and/or detection. FIG. 13B shows the presence of a covalent complex of Compound 73 with AKT1 E17K mutant protein (peaks corresponding to masses of 56374 and 56454 Da), and a small level of unmodified mutant protein (peaks corresponding to masses of 55850 and 55930 Da). By comparing FIG. 13A to FIG. 13B shows that Compound 73 is selective for complex formation with AKT1 E17K.

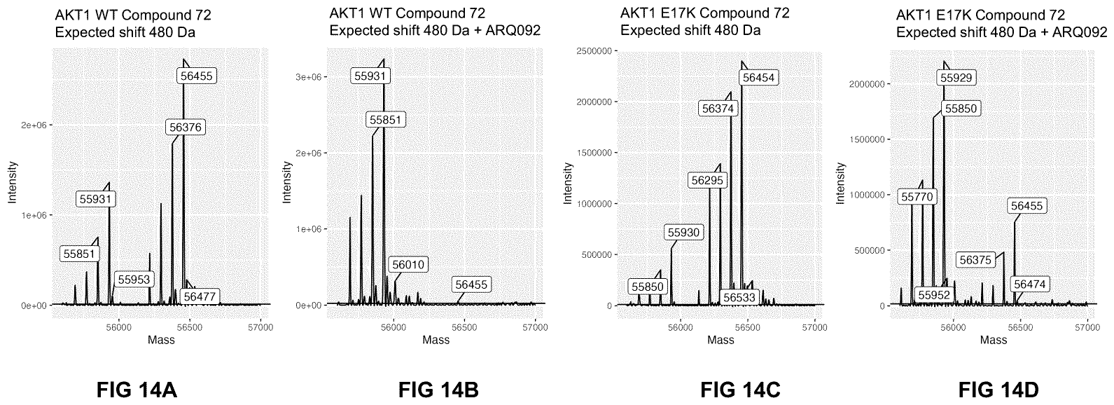

FIG. 14 provides intact mass spectra following incubation of Compound 72 with AKT1 WT protein or AKT1 E17K mutant protein, both in the absence of any competitor molecule (FIGS. 14A, 14C) and in the presence of a significant molar excess of competitor molecule ARQ-092 (FIGS. 14B, 14D). FIG. 14A shows a significant presence of a covalent complex of Compound 72 with AKT1 WT protein (peaks corresponding to masses of 56376 and 56455 Da), and some unmodified WT protein (peaks corresponding to masses of 55851 and 55931 Da). Essentially no covalent complex formation was observed in FIG. 14B, indicating that Compound 72 bound to the same AKT1 WT binding pocket as the competitor molecule. FIG. 14C shows a significant presence of a covalent complex of Compound 72 with AKT1 E17K mutant protein (peaks corresponding to masses of 56295,56374, and 56454 Da), and a small level of unmodified mutant protein (peaks corresponding to masses of 55850 and 55930 Da). In contrast, a significantly smaller level of covalent complex formation was observed in FIG. 14D, suggesting that Compound 72 bound to the same AKT1 E17K binding pocket as the competitor molecule.

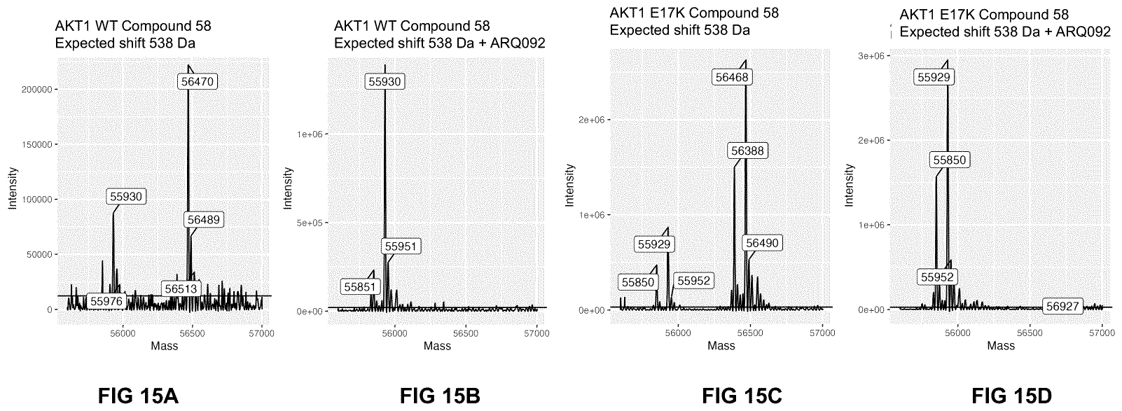

FIG. 15 provides intact mass spectra following incubation of Compound 58 with AKT1 WT protein or AKT1 E17K mutant protein, both in the absence of any competitor molecule (FIGS. 15A, 15C) and in the presence of a significant molar excess of competitor molecule ARQ-092 (FIGS. 15B, 15D). FIG. 15A shows the presence of a covalent complex of Compound 58 with AKT1 WT protein (peaks corresponding to masses of 56470 and 56489 Da), and some unmodified WT protein (peak corresponding to mass of 55930 Da). Essentially no covalent complex formation was observed in FIG. 15B, indicating that Compound 58 bound to the same AKT1 WT binding pocket as the competitor molecule. FIG. 15C shows a significant presence of a covalent complex of Compound 58 with AKT1 E17K mutant protein (peaks corresponding to masses of 56388 and 56468 Da), and unmodified mutant protein (peaks corresponding to masses of 55850 and 55929 Da). Essentially no covalent complex formation was observed in FIG. 15D, indicating that Compound 58 bound to the same AKT1 E17K binding pocket as the competitor molecule.

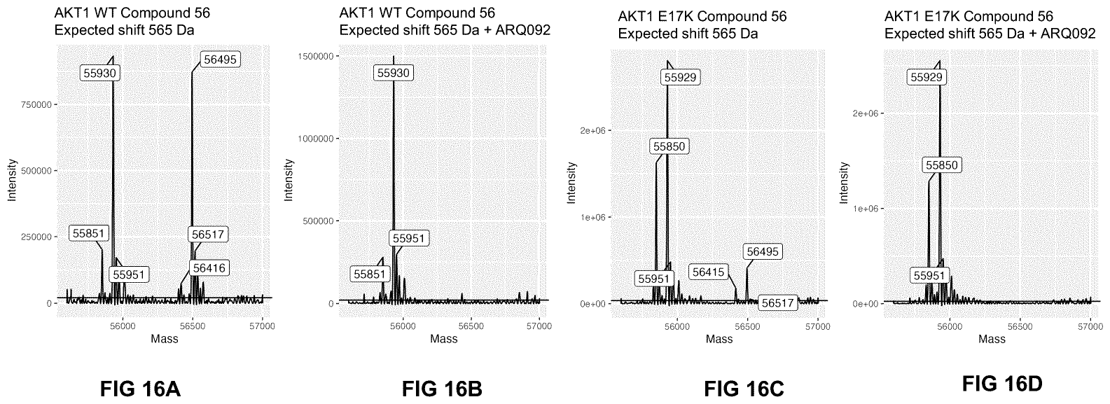

FIG. 16 provides intact mass spectra following incubation of Compound 56 with AKT1 WT protein or AKT1 E17K mutant protein, both in the absence of any competitor molecule (FIGS. 16A, 16C) and in the presence of a significant molar excess of competitor molecule ARQ-092 (FIGS. 16B, 16D). FIG. 16A shows the presence of a covalent complex of Compound 56 with AKT1 WT protein (peaks corresponding to masses of 56517 and 56495 Da), and unmodified protein (peaks corresponding to masses of 55851 and 55930 Da). Essentially no covalent complex formation was observed in FIG. 16B, indicating that Compound 56 bound to the same AKT1 WT binding pocket as the competitor molecule. FIG. 16C shows a small presence of a covalent complex of Compound 56 with AKT1 E17K mutant protein (peaks corresponding to masses of 56415 and 56495 Da), and a significant level of unmodified mutant protein (peaks corresponding to masses of 55850 and 55929 Da), indicating that either a low level of covalent complex was formed, or the resulting C—N bond mostly fragmented during ionization and/or detection. Essentially no covalent complex formation was observed in FIG. 16D.

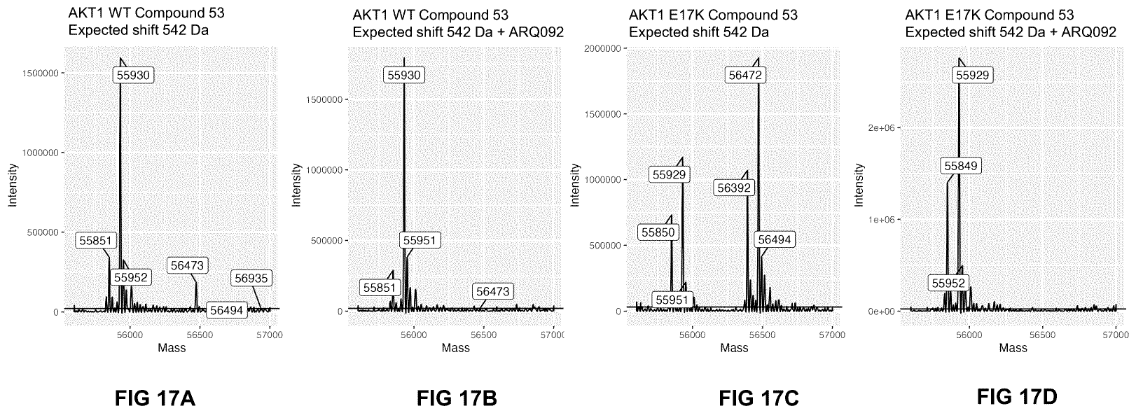

FIG. 17 provides intact mass spectra following incubation of Compound 53 with AKT1 WT protein or AKT1 E17K mutant protein, both in the absence of any competitor molecule (FIGS. 17A, 17C) and in the presence of a significant molar excess of competitor molecule ARQ-092 (FIGS. 17B, 17D). For the AKT1 WT protein ender both conditions, only peaks corresponding to unmodified WT protein (peaks corresponding to masses 55851 and 55930 Da) were observed for Compound 53, indicating that either little to no covalent complex with AKT1 WT was formed, or the resulting C—N bond completely fragmented during ionization and/or detection. FIG. 17C shows the presence of a covalent complex of Compound 53 with AKT1 E17K mutant protein (peaks corresponding to masses of 56392 and 56472 Da), and unmodified mutant protein (peaks corresponding to masses of 55850 and 55929 Da). Essentially no covalent complex formation was observed in FIG. 17D, indicating that Compound 53 bound to the same AKT1 E17K binding pocket as the competitor molecule. Comparing FIG. 17A to FIG. 17C shows that Compound 53 is selective for complex formation with AKT1 E17K.

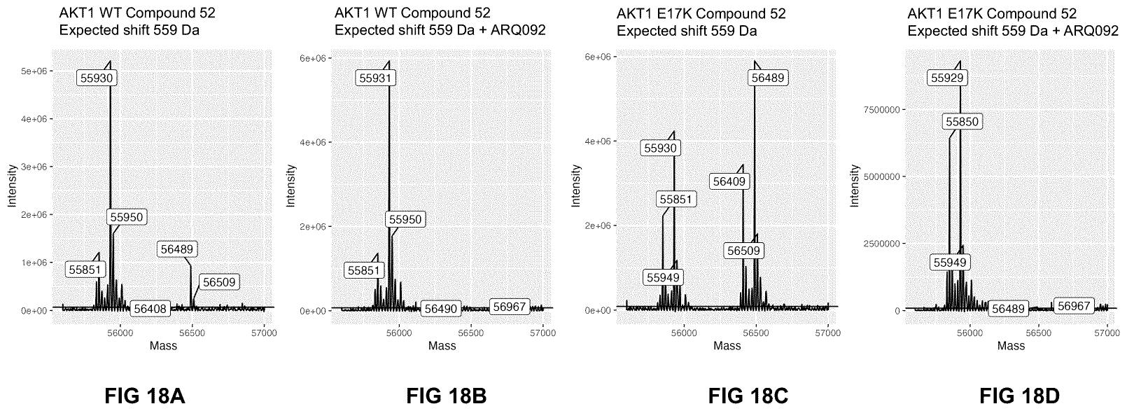

FIG. 18 provides intact mass spectra following incubation of Compound 52 with AKT1 WT protein or AKT1 E17K mutant protein, both in the absence of any competitor molecule (FIGS. 18A, 18C) and in the presence of a significant molar excess of competitor molecule ARQ-092 (FIGS. 18B, 18D). For the AKT1 WT protein ender both conditions, only peaks corresponding to unmodified WT protein (peaks corresponding to masses 55851 and 55930 Da) were observed for Compound 52, indicating that either little to no covalent complex with AKT1 WT was formed, or the resulting C—N bond completely fragmented during ionization and/or detection. FIG. 18C shows the presence of a covalent complex of Compound 52 with AKT1 E17K mutant protein (peaks corresponding to masses of 56409 and 56489 Da), and unmodified mutant protein (peaks corresponding to masses of 55851 and 55930 Da). Essentially no covalent complex formation was observed in FIG. 18D, indicating that Compound 52 bound to the same AKT1 E17K binding pocket as the competitor molecule. Comparing FIG. 18A to FIG. 18C shows that Compound 52 is selective for complex formation with AKT1 E17K.

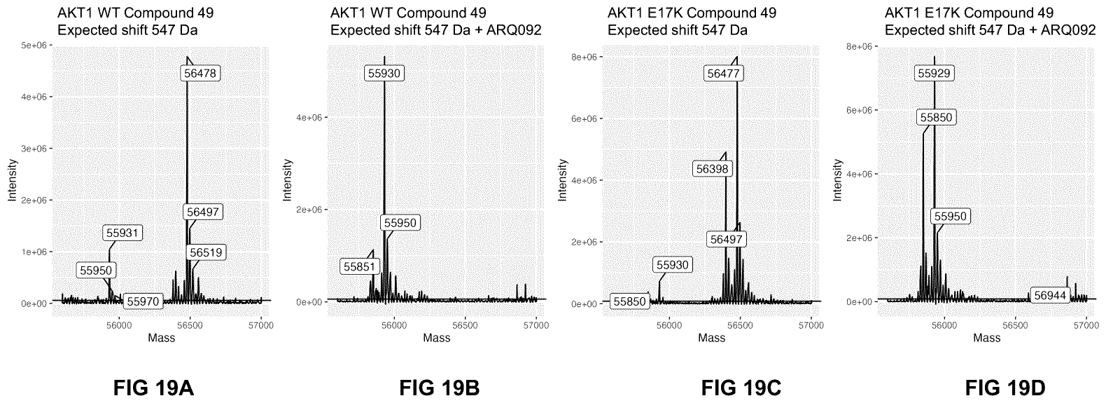

FIG. 19 provides intact mass spectra following incubation of Compound 49 with AKT1 WT protein or AKT1 E17K mutant protein, both in the absence of any competitor molecule (FIGS. 19A, 19C) and in the presence of a significant molar excess of competitor molecule ARQ-092 (FIGS. 19B, 19D). FIG. 19A shows a significant presence of covalent complex of Compound 49 with AKT1 WT protein (peaks corresponding to masses of 56478 and 56497 Da), and essentially little to no unmodified WT protein (peak corresponding to mass of 55931 Da). Essentially no covalent complex formation was observed in FIG. 19B, indicating that Compound 49 bound to the same AKT1 WT binding pocket as the competitor molecule. FIG. 19C shows a significant presence of a covalent complex of Compound 49 with AKT1 E17K mutant protein (peaks corresponding to masses of 56398 and 56477 Da), and essentially little to no unmodified mutant protein (peak corresponding to mass of 55930 Da). Essentially little to no covalent complex formation was observed in FIG. 19D, indicating that Compound 49 bound to the same AKT1 E17K binding pocket as the competitor molecule.

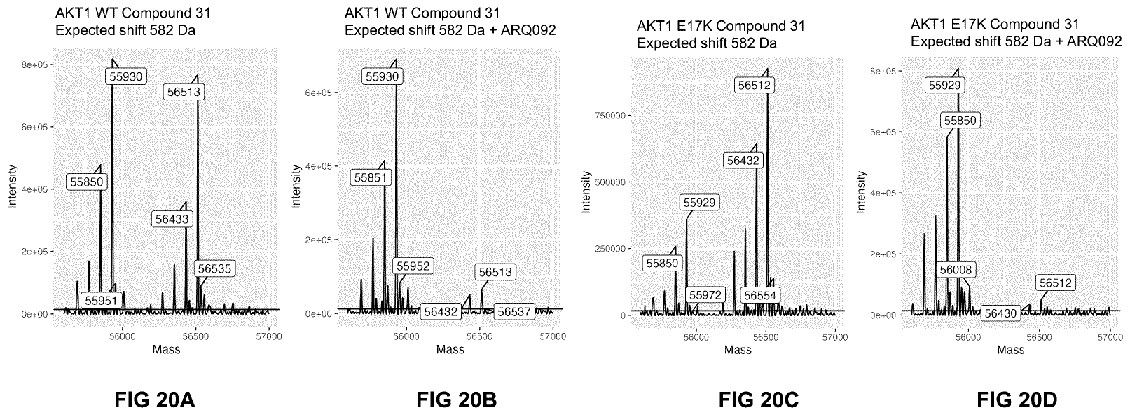

FIG. 20 provides intact mass spectra following incubation of Compound 31 with AKT1 WT protein or AKT1 E17K mutant protein, both in the absence of any competitor molecule (FIGS. 20A, 20C) and in the presence of a significant molar excess of competitor molecule ARQ-092 (FIGS. 20B, 20D). FIG. 20A shows presence of a covalent complex of Compound 31 with AKT1 WT protein (peaks corresponding to masses of 56433 and 56513 Da), and unmodified WT protein (peaks corresponding to masses of 55850 and 55930 Da). Essentially little to no covalent complex formation was observed in FIG. 20B, indicating that Compound 31 bound to the same AKT1 WT binding pocket as the competitor molecule. FIG. 20C shows a significant presence of a covalent complex of Compound 31 with AKT1 E17K mutant protein (peaks corresponding to masses of 56432 and 56512 Da), and a lower level of unmodified mutant protein (peaks corresponding to masses of 55850 and 55929 Da). Essentially little to no covalent complex formation was observed in FIG. 20D, suggesting that Compound 31 bound to the same AKT1 E17K binding pocket as the competitor molecule.

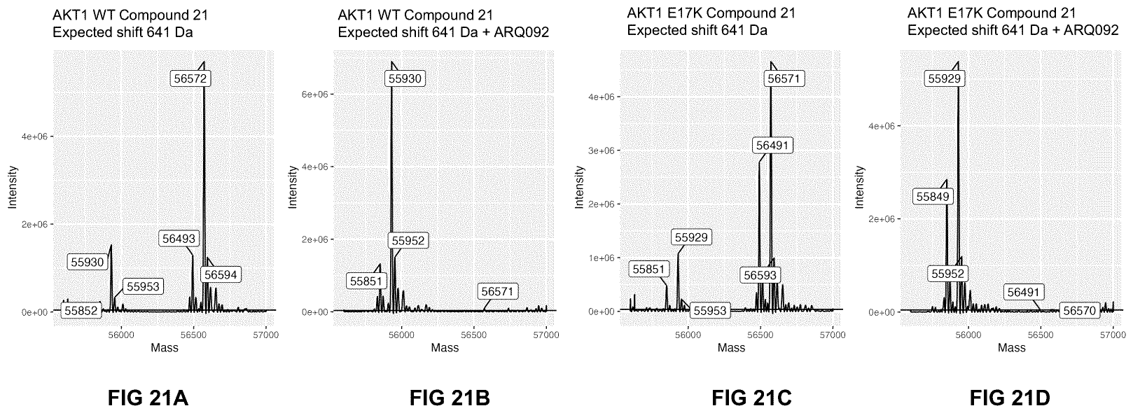

FIG. 21 provides intact mass spectra following incubation of Compound 21 with AKT1 WT protein or AKT1 E17K mutant protein, both in the absence of any competitor molecule (FIGS. 21A, 21C) and in the presence of a significant molar excess of competitor molecule ARQ-092 (FIGS. 21B, 21D). FIG. 21A shows a significant presence of a covalent complex of Compound 21 with AKT1 WT protein (peaks corresponding to masses of 56493 and 56572 Da), and a small level of unmodified WT protein (peak corresponding to mass of 55930 Da). Essentially no covalent complex formation was observed in FIG. 21B, indicating that Compound 21 bound to the same AKT1 WT binding pocket as the competitor molecule. FIG. 21C shows a significant presence of a covalent complex of Compound 21 with AKT1 E17K mutant protein (peaks corresponding to masses of 56491 and 56571 Da), and a small level of unmodified mutant protein (peaks corresponding to masses of 55851 and 55929 Da). Essentially no covalent complex formation was observed in FIG. 21D, suggesting that Compound 21 bound to the same AKT1 E17K binding pocket as the competitor molecule.

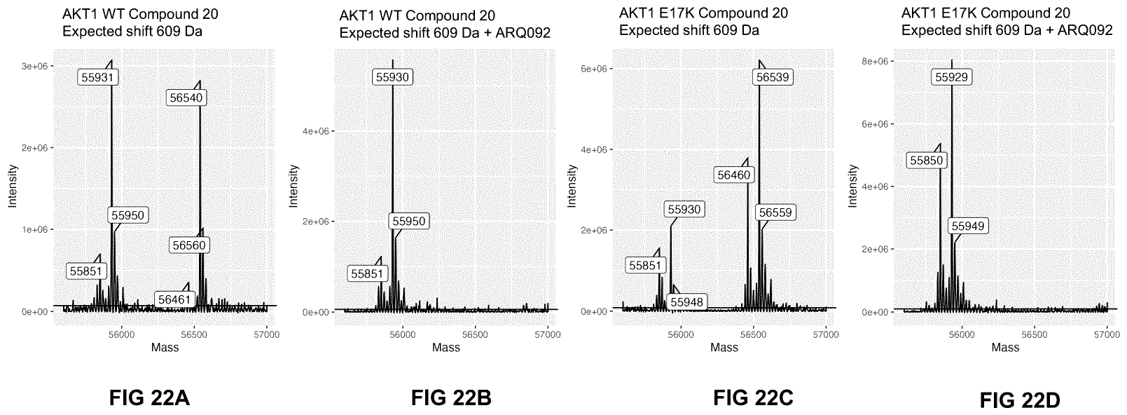

FIG. 22 provides intact mass spectra following incubation of Compound 20 with AKT1 WT protein or AKT1 E17K mutant protein, both in the absence of any competitor molecule (FIGS. 22A, 22C) and in the presence of a significant molar excess of competitor molecule ARQ-092 (FIGS. 22B, 22D). FIG. 22A shows presence of a covalent complex of Compound 20 with AKT1 WT protein (peaks corresponding to masses of 56540 and 56560 Da), and unmodified WT protein (peaks corresponding to masses of 55851 and 55931 Da). Essentially no covalent complex formation was observed in FIG. 22B, indicating that Compound 20 bound to the same AKT1 WT binding pocket as the competitor molecule. FIG. 22C shows a significant presence of a covalent complex of Compound 20 with AKT1 E17K mutant protein (peaks corresponding to masses of 56460 and 56539 Da), and a small level of unmodified mutant protein (peaks corresponding to masses of 55851 and 55930 Da). Essentially no covalent complex formation was observed in FIG. 22D, suggesting that Compound 20 bound to the same AKT1 E17K binding pocket as the competitor molecule. Comparing FIG. 22A to FIG. 22C shows that Compound 20 is selective for complex formation with AKT1 E17K.

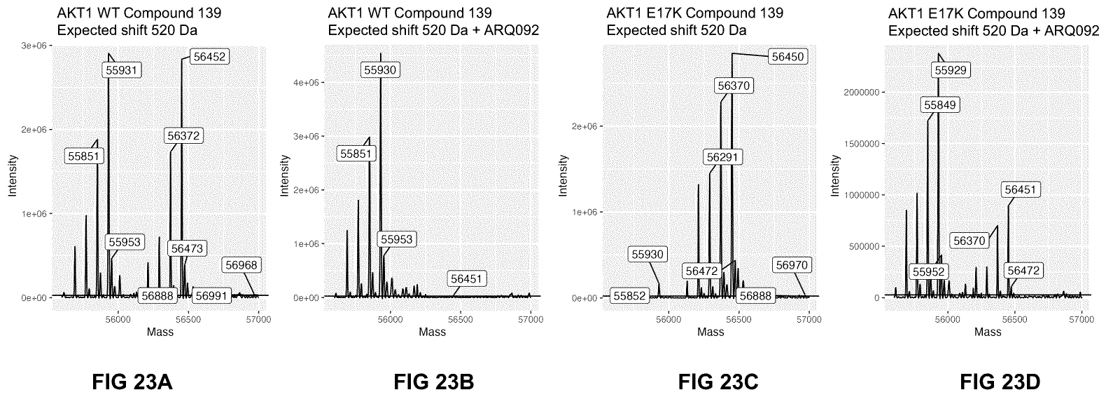

FIG. 23 provides intact mass spectra following incubation of Compound 139 with AKT1 WT protein or AKT1 E17K mutant protein, both in the absence of any competitor molecule (FIGS. 23A, 23C) and in the presence of a significant molar excess of competitor molecule ARQ-092 (FIGS. 23B, 23D). FIG. 23A shows presence of a covalent complex of Compound 139 with AKT1 WT protein (peaks corresponding to masses of 56372 and 56452 Da), and unmodified WT protein (peaks corresponding to masses of 55851 and 55931 Da). Essentially no covalent complex formation was observed in FIG. 23B, indicating that Compound 139 bound to the same AKT1 WT binding pocket as the competitor molecule. FIG. 23C shows a significant presence of a covalent complex of Compound 139 with AKT1 E17K mutant protein (peaks corresponding to masses of 56291, 56370 and 56450 Da), and a little to no unmodified mutant protein. In contrast, a lower level of covalent complex formation was observed in FIG. 23D, suggesting that Compound 139 bound to the same AKT1 E17K binding pocket as the competitor molecule. Comparing FIG. 23A to FIG. 23C shows that Compound 139 is selective for complex formation with AKT1 E17K.

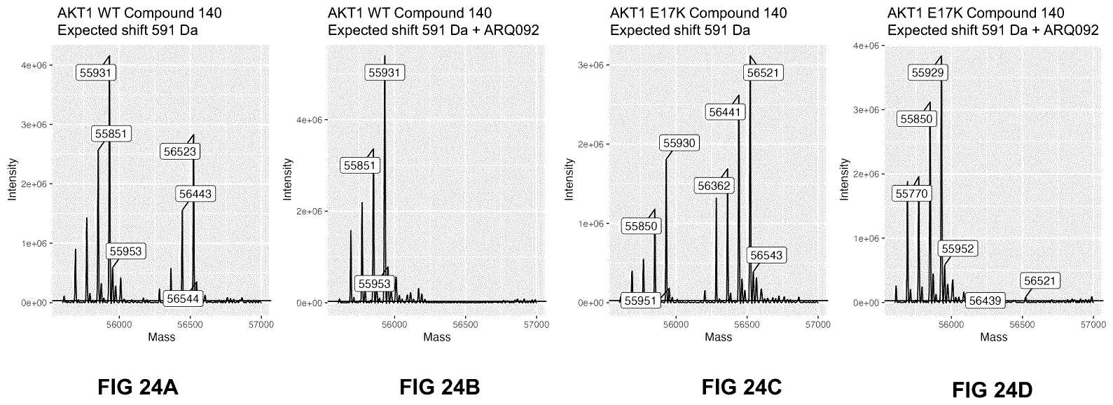

FIG. 24 provides intact mass spectra following incubation of Compound 140 with AKT1 WT protein or AKT1 E17K mutant protein, both in the absence of any competitor molecule (FIGS. 24A, 24C) and in the presence of a significant molar excess of competitor molecule ARQ-092 (FIGS. 24B, 24D). FIG. 24A shows presence of a covalent complex of Compound 140 with AKT1 WT protein (peaks corresponding to masses of 56443 and 56523 Da), and unmodified WT protein (peaks corresponding to masses of 55851 and 55931 Da). Essentially no covalent complex formation was observed in FIG. 24B, indicating that Compound 140 bound to the same AKT1 WT binding pocket as the competitor molecule. FIG. 24C shows a significant presence of a covalent complex of Compound 140 with AKT1 E17K mutant protein (peaks corresponding to masses of 56362, 56441, and 56521 Da), and unmodified mutant protein (peaks corresponding to masses of 55850 and 55930 Da). Essentially no covalent complex formation was observed in FIG. 24D, suggesting that Compound 140 bound to the same AKT1 E17K binding pocket as the competitor molecule.

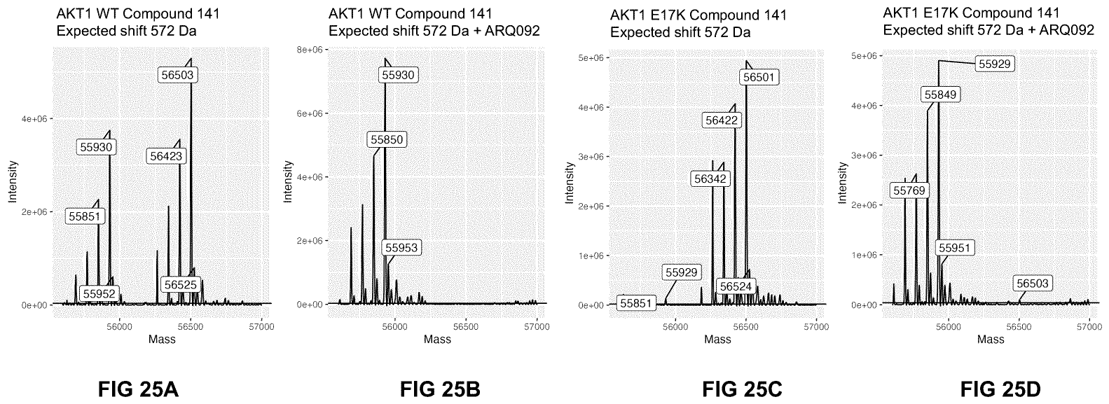

FIG. 25 provides intact mass spectra following incubation of Compound 141 with AKT1 WT protein or AKT1 E17K mutant protein, both in the absence of any competitor molecule (FIGS. 25A, 25C) and in the presence of a significant molar excess of competitor molecule ARQ-092 (FIGS. 25B, 25D). FIG. 25A shows presence of a covalent complex of Compound 141 with AKT1 WT protein (peaks corresponding to masses of 56423 and 56503 Da), and unmodified WT protein (peaks corresponding to masses of 55851 and 55931 Da). Essentially no covalent complex formation was observed in FIG. 25B, indicating that Compound 141 bound to the same AKT1 WT binding pocket as the competitor molecule. FIG. 25C shows a significant presence of a covalent complex of Compound 141 with AKT1 E17K mutant protein (peaks corresponding to masses of 56342, 56422, and 56501 Da), and essentially no unmodified mutant protein. Essentially no covalent complex formation was observed in FIG. 25D, suggesting that Compound 141 bound to the same AKT1 E17K binding pocket as the competitor molecule. Comparing FIG. 25A to FIG. 25C shows that Compound 141 is selective for complex formation with AKT1 E17K.

### Example 144: Dissociation Rate Data for compounds with AKT1 WT and AKT1 E17K

AKT1 WT or AKT1 E17K (1 μM) were treated with a compound of Table 1 (1.5 μM) for 2 hours at 37° C., then ARQ092 (50 μM final concentration) was added to prevent aldehyde-containing compound rebinding after initial dissociation. At various timepoints, aliquots were removed and quenched with 5 mM NaBH4 for 5 min to capture the reversible imine through reductive amination. Reduction reactions were quenched by diluting the sample in equal volume acetonitrile+1% TFA, then analyzed by LCMS. Deconvoluted monoisotopic intensities of unmodified vs. compound-modified mass peaks were compared.

TABLE 2
Half-life data for compounds

| | AKT1 | AKT1 | Off-rate |
|---|---|---|---|
| | (WT) | E17K | Selectivity |
| Compound No. | t_{1/2} (mins) | t_{1/2} (mins) | E17K/WT |
| 133 | 22.7 | 126 | 5.6 |
| 125 | 45.3 | 176 | 3.9 |
| 110 | 4.9 | 43.2 | 8.8 |
| 96 | 10.7 | 55.5 | 5.2 |
| 94 | 24.2 | 111 | 4.6 |
| 74 | 12 | 73.2 | 6.1 |
| 73 | 25 | 94.2 | 3.8 |
| 31 | 85.9 | 151 | 1.8 |
| 21 | 45.3 | 68.2 | 1.5 |
| 139 | 47.8 | — | — |

### Example 145: Crystallography Studies Showing Covalent Complex Formation

### Protein Expression and Purification

The following protein sequences were used for the described crystallography studies. Amino acid mutation numbers are based on the naturally occurring human AKT1 amino acid sequence and not the genetically modified sequence (e.g., the E17K mutation is shown as the 34^{th} residue in the following sequences).

AKT1-WT Amino Acid Sequence:
MSHHHHHHHHGSENLYFQSDVAIVKEGWLHKRGEYIKTWRPRYFLLKND
GTFIGYKERPQDVDQREAPLNNFSVAQCQLMKTERPRPNTFIIRCLQWT
TVIERTFHVETPEEREEWTTAIQTVADGLKKQEEEEMDASAEHTDMEVS
LAKPKHRVTMNEFEYLKLLGKGTFGKVILVKEKATGRYYAMKILKKEVI
VAKDEVAHTLTENRVLQNSRHPFLTALKYSFQTHDRLCFVMEYANGGEL
FFHLSRERVFSEDRARFYGAEIVSALDYLHSEKNVVYRDLKLENLMLDK
DGHIKITDFGLCKEGIKDGATMKTFCGTPEYLAPEVLEDNDYGRAVDWW
GLGVVMYEMMCGRLPFYNQDHEKLFELILMEEIRFPRTLGPEAKSLLSG
LLKKDPKQRLGGGSEDAKEIMQHRFFAGIVWQHVYEKKLSPPFKPQVTS
ETDTRYFDEEFTAQM
AKT1-E17K Sequence:
MSHHHHHHHHGSENLYFQSDVAIVKEGWLHKRGKYIKTWRPRYFLLKND
GTFIGYKERPQDVDQREAPLNNFSVAQCQLMKTERPRPNTFIIRCLQWT
TVIERTFHVETPEEREEWTTAIQTVADGLKKQEEEEMDASAEHTDMEVS
LAKPKHRVTMNEFEYLKLLGKGTFGKVILVKEKATGRYYAMKILKKEVI
VAKDEVAHTLTENRVLQNSRHPFLTALKYSFQTHDRLCFVMEYANGGEL
FFHLSRERVFSEDRARFYGAEIVSALDYLHSEKNVVYRDLKLENLMLDK
DGHIKITDFGLCKEGIKDGATMKTFCGTPEYLAPEVLEDNDYGRAVDWW
GLGVVMYEMMCGRLPFYNQDHEKLFELILMEEIRFPRTLGPEAKSLLSG
LLKKDPKQRLGGGSEDAKEIMQHRFFAGIVWQHVYEKKLSPPFKPQVTS
ETDTRYFDEEFTAQM
NB41 Amino Acid Sequence:
QVQLQESGGGLVQAGGSLRLSCAASGIDVRIKTMAWYRQAPGKQRELLA
SVLVSGSTNYADPVKGRFTISRDNAKNTVYLQMNKLIPDDTAVYYCNTY
GRLRRDVWGPGTQVTVSSHHHHHHEPEA

AKT1 WT and AKT1 E17K were gene synthesized and cloned by Gibson assembly into the pFastBac vector. The plasmid was transformed into DH10Bac cells and the recombinant bacmid was isolated and used to generate baculovirus in Sf9 cells.

Expression of AKT1 was induced in Sf9 cells by infection of 1.0 L of cultured cells (1.1 M/mL) with 20 mL of baculovirus solution and incubated for 72 h. The cells were harvested (3500 rpm, 10 min), and the pellets were resuspended in 100 mL lysis buffer (25 mM HEPES, pH 8.0, 150 mM NaCl, 1.0 mM TCEP) supplemented with protease inhibitor cocktail (Roche). The cells were lysed by sonication and the cell debris pelleted by centrifugation (20000 rpm, 30 min). The clarified lysate was incubated with NiNTA (3.0 mL) and the resin was washed with lysis buffer (3×10 mL) and then 5 mL wash buffer (40 mM imidazole, 25 mM HEPES, pH 8.0, 150 mM NaCl, 1.0 mM TCEP). AKT1 was eluted in 6 mL of elution buffer (400 mM imidazole, 25 mM Hepes, pH 8.0, 150 mM NaCl, 1.0 mM TCEP). To the eluted protein was added 1.5 mg TEV protease. After 1 hour, LCMS showed complete TEV cleavage. The resulting solution was diluted to 60 mL with 20 mM Tris pH 8.0, 150 mM NaCl and then passed through Ni-NTA (2 mL) and the flowthrough collected. The resin was further washed with 5 mL of 25 mM Tris pH 7.5, 100 mM NaCl, 40 mM imidazole buffer to recover the remaining AKT1 which was combined with the flowthrough. The protein was buffer exchanged into 20 mM Tris pH 8.0 20 mM NaCl and purified by ion exchange chromatography (Q HiTrap, gradient from 20 to 250 mM NaCl). The resulting protein was purified further by gel filtration on a Superdex 200 (16/60) column (gel filtration buffer: 25 mM HEPES pH 8.0, 150 mM NaCl, 1 mM TCEP). The protein was then concentrated to 5 mg/mL, flash-frozen in liquid nitrogen and stored at −80° C. The final yield was ˜4 mg AKT1.

NB41 was gene synthesized and cloned by Gibson assembly into a PET26b vector containing an N-terminal PelB signal sequence. BL21 cells were transformed with the NB41 PET26b plasmid. The transformed bacteria were grown in 2 L TB+kanamycin at 37° C. until it reached OD600=0.66. The culture was cooled to 18° C., induced with 0.25 mM IPTG and expressed overnight at 18° C. The bacteria were harvested (5000 rpm, 10 min) and the resulting pellet frozen.

The pellet was warmed to 4° C. and lysed in 15 mL TS buffer (TS buffer=200 mM Tris pH 8.0, 500 mM sucrose) with rotation. After 1 hour, 30 mL of TS/4 buffer was added (TS/4 buffer=50 mM Tris, 125 mM Sucrose). After a further 45 min rotation at 4° C., the sample was centrifuged for 30 min at 7500 g. The supernatant was collected and passed over 3 mL NiNTA resin. The resin was washed with 3×10 mL wash buffer (40 mM imidazole, 25 mM HEPES, pH 8.0, 150 mM NaCl). NB41 was eluted in 6 mL of elution buffer (400 mM imidazole, 25 mM Hepes, pH 8.0, 150 mM NaCl). The resulting protein was purified further by gel filtration on a Superdex 75 (16/60) column (gel filtration buffer: 20 mM Tris pH 7.5, 100 mM NaCl, 1 mM TCEP). The protein was then concentrated to 5 mg/mL and flash-frozen in liquid nitrogen and stored at −80° C.

### AKT1-Inhibitor-NB41 Complex Formation

To 1 mg of AKT1 WT or AKT1 E17K in 10 mL of 20 mM HEPES (pH 8.0), 150 mM NaCl, 1 mM TCEP buffer was added 200 μL Compound 110 or Compound 133 (0.5 mM stock in DMSO) to give a final concentration of 10 μM. After 10 min, the sample was concentrated to 1 mL and then 0.55 mg NB41 was added and incubated for 15 min. The resulting complex was purified by gel filtration on a Superdex 200 (16/60) column (gel filtration buffer: 20 mM HEPES pH 8.0, 20 mM NaCl, 1 mM TCEP). The protein was then concentrated to 6.2-7.5 mg/mL and used directly for crystal formation.

### Crystallization, Data Collection and Structure Determination

AKT1-E17K/Compound 133: AKT1 E17K at 3 mg/ml were incubated with Compound 133 at 1:5 molar ratio on ice for 1 hr. Co-crystals of AKT1-E17K/Compound 133 were obtained at 277K from hanging drops using reservoir solutions containing 1.25 mM Na-acetate 6.0, 3.75 mM sodium citrate 7.5, 15% PEG MME 2000. Co-crystals were flash cooled in liquid nitrogen after addition of 20% ethylene glycol supplemented to the mother liquor. Diffraction data of AKT1-E17K/Compound 133 was obtained at 100 K at beamline 08B1 in CLS, using an Pilatus3 S 6M detector (DECTRIS Ltd.). These datasets were further processed with autoPROC (Global Phasing). The initial structures were determined through molecular replacement with Phaser in CCP4 suite with the structure of PDB ID 5kcv as a search model. A chemical restraint dictionary was generated using ACEDRG and CCP4i2. The models were manually adjusted using Coot, refined with REFMAC5 to a final resolution of 3.0 Å for the AKT1-E17K/Compound 133 complex.

AKT1-WT/Compound 133-NB41: The protein solution: AKT1-WT/Compound 133-NB41 (7.5 mg/mL) in 20 mM HEPES (pH 8.0), 20 mM NaCl, 1 mM TCEP, 0.4 mM iodoacetamide; and the reservoir solution: 19% PEG3350, 200 mM Li₂SO₄, 100 mM BisTris Propane pH 7.2, 10% ethylene glycol; were mixed in a 1:1 ratio and the crystals grown overnight and were cryoprotected in the reservoir solution containing 25% ethylene glycol.

AKT1-E17K/Compound 110-NB41: The protein solution: AKT1-E17K/Compound 110-NB41 (6.2 mg/mL) in 20 mM HEPES (pH 8.0), 20 mM NaCl, 1 mM TCEP; and reservoir solution: 18% PEG3350, 200 mM Li2SO4, 100 mM BisTris Propane pH 7.8, 10% ethylene glycol; were mixed in a 1:1 ratio and the crystals grown overnight were cryoprotected in the reservoir solution containing 25% ethylene glycol.

Diffraction data were collected at beamline 2.0.1 of the Advanced Light Source (Lawrence Berkeley National Laboratory) and indexed and integrated using iMosflm, scaled with Scala and solved by molecular replacement using Phaser. The structure was manually refined with Coot and PHENIX.

TABLE 3
Data collection and refinement statistics:

| | AKT1-E17K/ | | AKT1-WT/ | | AKT1-E17K/ | |
|---|---|---|---|---|---|---|
| | Compound 133 | | Compound 133-NB41 | | Compound 110-NB41 | |
| Wavelength | 1.181 | | 1.00 | | 1.00 | |
| Resolution range | 45.79-2.7 | (2.797-2.7) | 57.57-1.97 | (2.04-1.97) | 43.60-2.45 | (2.54-2.45) |
| Space group | P 2₁ 2₁ 2₁ | | C 2 2 2₁ | | C 2 2 2₁ | |
| Unit cell | 70.61 71.08 91.58 | | 76.31 87.71 198.50 | | 77.27 89.35 199.64 | |
| | 90 90 90 | | 90 90 90 | | 90 90 90 | |
| Total reflections | 116944 | (7620) | 617827 | (46653) | 174097 | (17796) |
| Unique reflections | 12864 | (1167) | 47021 | (4345) | 25808 | (2523) |
| Multiplicity | 9.1 | (6.5) | 13.1 | (10.7) | 6.7 | (7.1) |
| Completeness (%) | 97.07 | (86.43) | 99.2 | (92.0) | 97.3 | (80.4) |
| Mean I/sigma(I) | 6.47 | (0.56) | 24.42 | (1.10) | 8.69 | (0.42) |
| Wilson B-factor | 64.96 | | 46.49 | | 65.37 | |
| R-merge | 0.2577 | (2.996) | 0.071 | (1.953) | 0.235 | (5.082) |
| R-meas | 0.2736 | (3.254) | 0.074 | (2.051) | 0.255 | (5.486) |
| R-pim | 0.08991 | (1.223) | 0.020 | (0.609) | 0.098 | (2.049) |
| CC1/2 | 0.995 | (0.212) | 1.000 | (0.442) | 0.993 | (0.054) |
| CC* | 0.999 | (0.591) | 1.000 | (0.783) | 0.998 | (0.320) |
| Reflections used | 12792 | (1102) | 46975 | (4310) | 25124 | (2028) |
| in refinement | | | | | | |
| Reflections used | 638 | (57) | 2000 | (185) | 1231 | (102) |
| for R-free | | | | | | |
| R-work | 0.2724 | (0.3665) | 0.217 | (0.329) | 0.227 | (0.367) |
| R-free | 0.3339 | (0.4882) | 0.261 | (0.359) | 0.299 | (0.368) |
| CC(work) | 0.915 | (0.433) | 0.950 | (0.643) | 0.957 | (0.270) |
| CC(free) | 0.865 | (0.379) | 0.925 | (0.543) | 0.928 | (0.170) |

| | | | |
|---|---|---|---|
| Number of | 3333 | 4342 | 4170 |
| non-hydrogen | | | |
| macromolecules | 3261 | 4160 | 4111 |
| ligands | 40 | 63 | 58 |
| solvent | 32 | 119 | 7 |
| Protein | 394 | 515 | 512 |
| residues | | | |
| RMS(bonds) | 0.005 | 0.009 | 0.009 |
| RMS(angles) | 1.04 | 1.12 | 1.06 |
| Ramachandran | 88.86 | 95.01 | 89.40 |
| favored (%) | | | |
| Ramachandran | 9.07 | 4.59 | 10.20 |
| allowed (%) | | | |
| Ramachandran | 2.07 | 0.40 | 0.40 |
| outliers (%) | | | |
| Rotamer | 2.57 | 2.05 | 6.45 |
| outliers (%) | | | |
| Clashscore | 11.30 | 9.37 | 27.08 |
| Average | 61.90 | 58.90 | 73.64 |
| B-factor | | | |
| macromolecules | 62.29 | 59.04 | 73.76 |
| ligands | 39.71 | 60.47 | 66.13 |
| solvent | 49.90 | 53.44 | 56.33 |

Statistics for the highest-resolution shell are shown in parentheses.

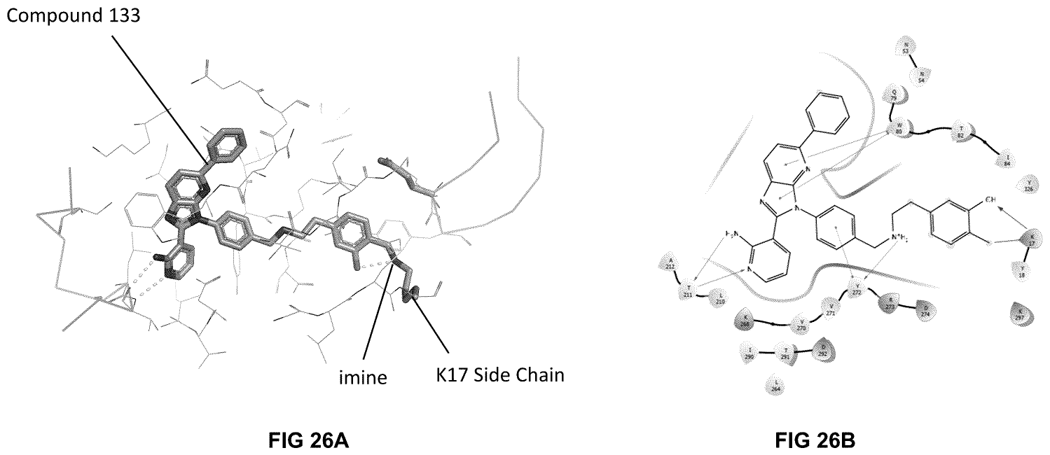

FIG. 26A is a close-up from the crystal structure of co-crystallization of AKT ET17K and Compound 133). In FIG. 26A, Compound 133 and the side chain of K17 of AKT1 E17K are represented by thicker stick model and the adjacent residues of AKT1 E17K are represented by a thinner stick model. Hydrogen bonds are depicted by dashed lines. FIG. 26A shows the imine bond formed between the nitrogen of K17 and the aldehyde of Compound 133, as well as the hydrogen bond between the imine nitrogen lone pair and the adjacent phenol hydrogen. The crystal structure provided in FIG. 26A thereby depicts the formation of a covalent complex between Compound 133 and K17 of AKT1 E17K.

FIG. 26B provides a 2-D diagram detailing the interactions between Compound 133 and residues of the AKT1 E17K protein. The imine bond is depicted by the line between K17 and the methylene of Compound 133. The hydrogen bond between the imine of K17 and the phenol of Compound 133 is depicted by the adjacent arrow. In addition to the covalent bond between K17 and Compound 133, additional hydrogen bonds are identified, including those between T211 and the aminopyridine nitrogen lone pair, as well as the adjacent primary amine group on Compound 133; and between Y272 and the secondary amine group on Compound 133. Pi stacking interactions are shown connecting W80 to the benzimidazole core and also connecting the Y272 and the phenylene. Further, Van der Waals interactions between the binding pocket residues and Compound 133 are also depicted.

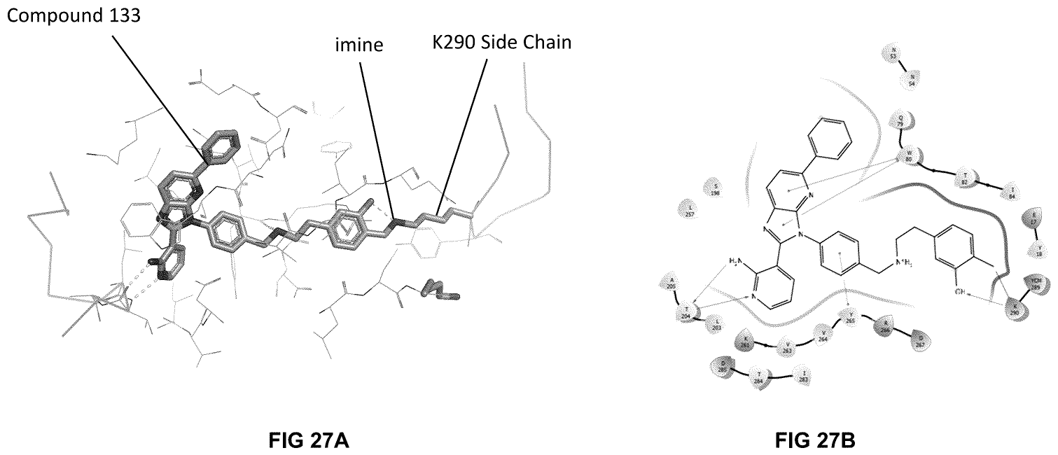

FIG. 27A is a close-up from the crystal structure of co-crystallization of AKT1 WT, Compound 133, and NB41). In FIG. 27A, Compound 133 and the side chain of K290 of AKT1 WT are represented by thicker stick model and the adjacent residues of AKT1 WT are represented by a thinner stick model. Hydrogen bonds are depicted by dashed lines. FIG. 27A shows the imine bond formed between the nitrogen of K290 and the aldehyde of Compound 133, as well as the hydrogen bond between the imine nitrogen lone pair and the adjacent phenol hydrogen. The crystal structure provided in FIG. 27A thereby depicts the formation of a covalent complex between Compound 133 and K290 of AKT1 WT.

FIG. 27B provides a 2-D diagram detailing the interactions between Compound 133 and residues of the AKT1 WT protein. The imine bond is depicted by the line between K290 and the methylene of Compound 133. The hydrogen bond between the imine of K290 and the phenol of Compound 133 is depicted by the adjacent arrow. In addition to the covalent bond between K290 and Compound 133, additional hydrogen bonds are identified, including those between T204 and the aminopyridine nitrogen lone pair. Pi stacking interactions are shown connecting W80 to the benzimidazole core and also connecting the Y265 and the phenylene. Further, Van der Waals interactions between the binding pocket residues and Compound 133 are also depicted.

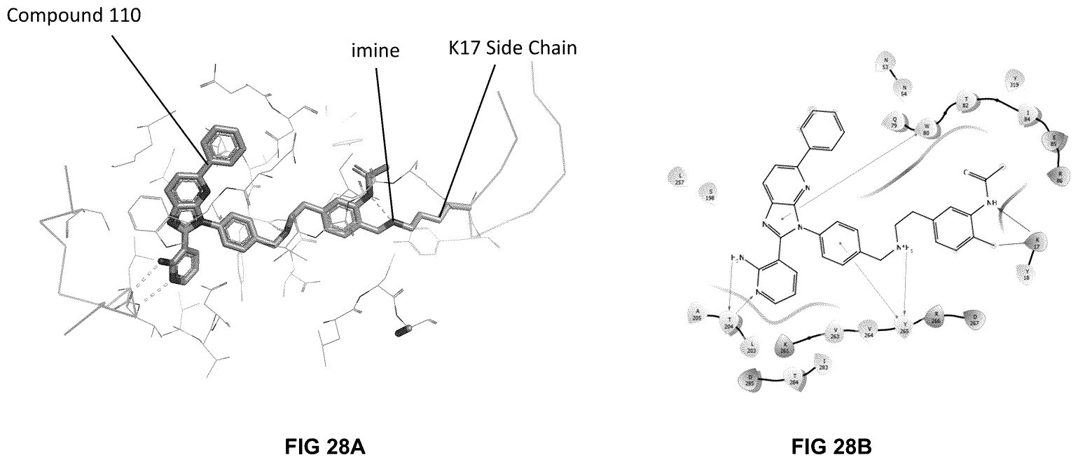

FIG. 28A is a close-up from the crystal structure of co-crystallization of AKT1 E17K and Compound 110). In FIG. 28A, Compound 110 and the side chain of K17 of AKT1 E17K are represented by thicker stick model and the adjacent residues of AKT1 E17K are represented by a thinner stick model. Hydrogen bonds are depicted by dashed lines. FIG. 28A shows the imine bond formed between the nitrogen of K17 and the aldehyde of Compound 110, as well as the hydrogen bond between the imine nitrogen lone pair and the adjacent amide hydrogen. The crystal structure provided in FIG. 28A thereby depicts the formation of a covalent complex between Compound 110 and K17 of AKT1 E17K.

FIG. 28B provides a 2-D diagram detailing the interactions between Compound 110 and residues of the AKT1 E17K protein. The imine bond is depicted by the line between K17 and the methylene of Compound 110. The hydrogen bond between the imine of K17 and the amide of Compound 110 is depicted by the adjacent arrow. In addition to the covalent bond between K17 and Compound 110, additional hydrogen bonds are identified, including those between T204 and the aminopyridine nitrogen lone pair, as well as the adjacent primary amine group on Compound 110; and between Y265 and the secondary amine group on Compound 110. Pi stacking interactions are shown connecting W80 to the benzimidazole core and also connecting the Y265 and the phenylene. Further, Van der Waals interactions between the binding pocket residues and Compound 110 are also depicted.

### Example 146: AKT1 Inhibition Data

Live cell target engagement assays were performed as described (Vasta, J. D. et al Cell Chem Biol 2018, 25(11), 206-214). Briefly, HEK293 cells were transfected with plasmids encoding kinase-NanoLuciferase fusion proteins overnight (Promega). Cells were then treated with serial dilutions of compound and an ˜EC50 concentration of fluorescently-tagged ATP-competitive tracer (Promega). After incubation for 2 hours at 37° C., luciferase substrate and extracellular luciferase inhibitor were added to all wells, and luminescent intensity at 460 nm and 600 nm were measured on a multimode plate-reader. The ratio of E₆₀₀/E₄₆₀ was calculated to give the tracer engagement signal (BRET). IC50 values were calculated by fitting BRET values to a log(inhibitor) vs. response Hill equation.

TABLE 4
IC₅₀ values and AKT1 E17K selectivity for compounds,
with compounds less than or equal to 100 nM as A; 100
nM > B ≥ 500 nM as B; and greater than 500 nM as C

| | AKT1 | AKT1 | | AKT1 E17K | AKT1 E17K |
|---|---|---|---|---|---|
| | E17K | WT | AKT2 | IC₅₀: | IC₅₀: |
| Compound | IC₅₀ | IC₅₀ | IC₅₀ | Selectivity | Selectivity |
| # | (nM) | (nM) | (nM) | vs AKT2 | vs AKT1 WT |
| 1 | A | A | C | 85 | 6.1 |
| 2 | A | B | C | 19 | 3.1 |
| 3 | B | B | C | 36 | 2.2 |
| 4 | A | A | C | 38 | 2 |
| 5 | A | A | C | 32 | 2.9 |
| 6 | B | C | C | 18 | 8.7 |
| 7 | A | B | C | 110 | 5.8 |
| 8 | A | B | C | 120 | 12 |
| 9 | A | C | C | >160 | 18 |
| 10 | A | A | C | 160 | 3.9 |
| 11 | A | A | B | 28 | 3.1 |
| 12 | A | A | B | 5.2 | 1.5 |
| 13 | A | A | A | 3 | 1.2 |
| 14 | A | A | B | 36 | 3.3 |
| 15 | A | B | C | 13 | 2 |
| 16 | A | B | C | 41 | 2.4 |
| 17 | A | C | C | >110 | 10 |
| 18 | A | B | C | 150 | 6.1 |
| 19 | A | A | A | 4.9 | 1.7 |
| 20 | A | A | A | 6 | 2 |
| 21 | A | A | A | 4.8 | 1.5 |
| 22 | A | A | C | 190 | 3.1 |
| 23 | A | A | C | 270 | 4.8 |
| 24 | A | A | C | 210 | 4.3 |
| 25 | A | A | C | 120 | 4.3 |
| 26 | A | A | C | 190 | 2.4 |
| 27 | A | B | C | 110 | 3.8 |
| 28 | A | A | B | 17 | 1.3 |
| 29 | A | A | B | 23 | 3 |
| 30 | A | B | C | 6.2 | 1.9 |
| 31 | A | A | A | 5.8 | 1.4 |
| 32 | A | B | C | 110 | 9 |
| 33 | A | B | C | 400 | 23 |
| 34 | A | B | C | 40 | 10 |
| 35 | A | A | B | 140 | 3.4 |
| 36 | A | A | B | 9.2 | 2 |
| 37 | A | A | C | 180 | 11 |
| 38 | B | B | C | 15 | 1.5 |
| 39 | A | B | B | 5.2 | 1.5 |
| 40 | A | A | B | 6.7 | 2.2 |
| 41 | C | C | C | 0.77 | 0.24 |
| 42 | A | A | A | 4.8 | 1.7 |
| 43 | A | B | C | >150 | 4.5 |
| 44 | B | C | C | 34 | 3.6 |
| 45 | C | C | C | >7.7 | 2.7 |
| 46 | C | C | C | <=>1.0 | <=>1.0 |
| 47 | C | C | C | 3.7 | 1.8 |
| 48 | A | B | C | 120 | 8.5 |
| 49 | A | A | C | 1500 | 20 |
| 50 | A | A | B | 32 | 2.1 |
| 51 | A | B | C | 90 | 3.8 |
| 52 | A | A | B | 11 | 1.2 |
| 53 | A | A | B | 48 | 2.7 |
| 54 | A | A | B | 64 | 1.8 |
| 55 | A | A | A | 6.8 | 0.93 |
| 56 | A | C | C | 490 | 93 |
| 57 | B | B | C | 13 | 2.7 |
| 58 | A | A | C | 410 | 8.6 |
| 59 | B | C | C | 39 | 5.7 |
| 60 | A | B | C | 45 | 8.2 |
| 61 | A | A | C | 66 | 3 |
| 62 | A | A | C | 47 | 2.7 |
| 63 | A | A | B | 8.3 | 1.2 |
| 64 | A | A | B | 2 | 0.5 |
| 65 | A | A | B | 37 | 1.2 |
| 66 | A | A | C | 830 | 3.5 |
| 67 | A | C | C | >240 | 27 |
| 68 | A | A | C | 150 | 4 |
| 69 | A | C | C | >160 | 16 |
| 70 | A | B | C | 65 | 7.7 |
| 71 | A | A | B | 37 | 5.8 |
| 72 | A | A | A | 18 | 1.7 |
| 73 | A | A | B | 22 | 2.1 |
| 74 | A | A | C | 130 | 5.5 |
| 75 | B | B | C | 3.7 | 1.7 |
| 76 | B | B | B | 4.7 | 1.6 |
| 77 | A | B | C | 39 | 4.3 |
| 78 | A | B | C | 43 | 2.2 |
| 79 | A | A | C | 88 | 4.4 |
| 80 | B | B | C | 2.5 | 0.47 |
| 81 | B | B | B | 1.7 | 0.51 |
| 82 | A | C | C | >400 | 29 |
| 83 | A | B | C | 99 | 10 |
| 84 | C | B | C | 4.8 | 0.22 |
| 85 | A | A | B | 6.5 | 1.3 |
| 86 | A | C | C | 74 | 25 |
| 87 | C | C | C | 1.6 | 0.72 |
| 88 | A | A | B | 55 | 3.9 |
| 89 | C | B | C | 2.1 | 0.14 |
| 90 | A | A | A | 1.6 | 0.65 |
| 91 | A | B | C | 120 | 5 |
| 92 | B | B | B | 0.75 | 0.26 |
| 93 | A | B | C | 50 | 3.9 |
| 94 | A | A | B | 22 | 2.5 |
| 95 | C | C | C | 2.5 | 1.8 |
| 96 | A | A | C | 64 | 4.6 |
| 97 | A | A | B | 1.9 | 0.56 |
| 98 | A | A | C | 38 | 2.5 |
| 99 | A | B | C | 8.4 | 3.7 |
| 100 | B | B | C | 13 | 1.9 |
| 101 | A | A | B | 5.9 | 1.8 |
| 102 | B | B | C | 2 | 1.1 |
| 103 | A | A | B | 11 | 2.5 |
| 104 | C | C | C | 1 | 0.72 |
| 105 | C | C | C | >1.0 | 1 |
| 106 | C | C | C | 0.91 | 0.85 |
| 107 | A | B | C | 76 | 3.5 |
| 108 | A | A | A | 2.1 | 1.2 |
| 109 | A | A | A | 0.6 | 0.63 |
| 110 | A | A | A | 5.3 | 1.2 |
| 111 | A | A | B | 6.3 | 0.83 |
| 112 | C | C | C | 1 | 1 |
| 113 | A | B | C | 57 | 2.5 |
| 114 | A | B | C | 14 | 2.2 |
| 115 | A | B | B | 4.9 | 1.2 |
| 116 | A | A | B | 10 | 1.9 |
| 117 | B | A | B | 2 | 0.47 |
| 118 | A | A | B | 29 | 1.6 |
| 119 | A | B | C | 18 | 2.9 |
| 120 | A | A | B | 2.3 | 0.86 |
| 121 | A | A | B | 6.2 | 2 |
| 122 | A | A | A | 3.2 | 1.3 |
| 123 | A | A | B | 2.2 | 1 |
| 124 | B | C | C | 13 | 3.1 |
| 125 | B | B | B | 0.39 | 0.28 |
| 126 | A | A | B | 12 | 5 |
| 127 | A | A | B | 7.7 | 2.6 |
| 128 | B | A | B | 0.93 | 0.15 |
| 129 | A | A | A | 2.5 | 0.51 |
| 130 | A | A | B | 7 | 1.5 |
| 131 | A | A | B | 13 | 1.6 |
| 132 | A | A | B | 23 | 3.2 |
| 133 | A | A | B | 4.7 | 1.4 |
| 134 | C | C | C | 4.3 | 2.3 |
| 135 | A | A | A | 0.72 | 0.16 |
| 136 | A | A | A | 1.9 | 1.3 |
| 137 | A | A | B | 4.1 | 0.91 |
| 138 | A | A | | | 0.52 |
| 139 | A | A | B | 20 | 1.7 |
| 140 | A | A | B | 10 | 1.4 |
| 141 | A | A | A | 4.4 | 1.2 |

### Example 147: Covalent Labeling Studies

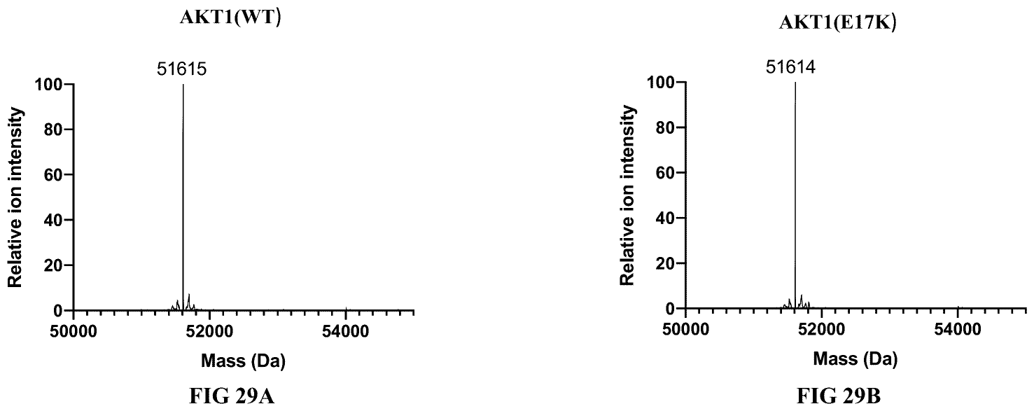

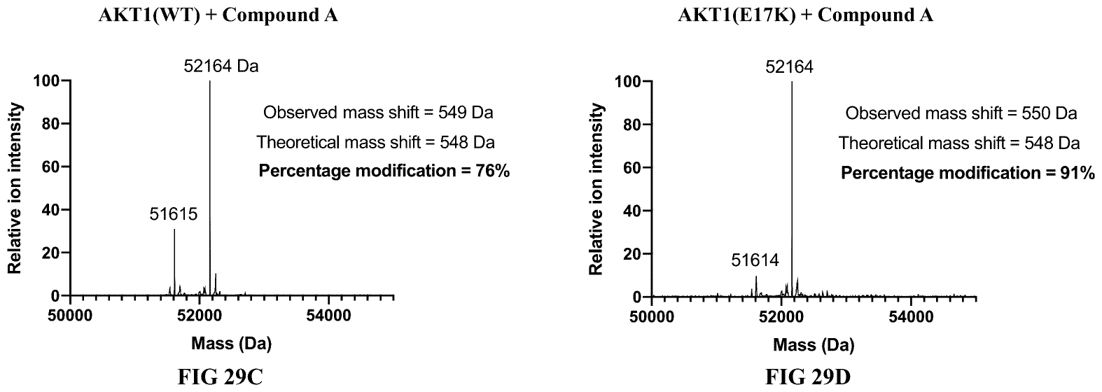

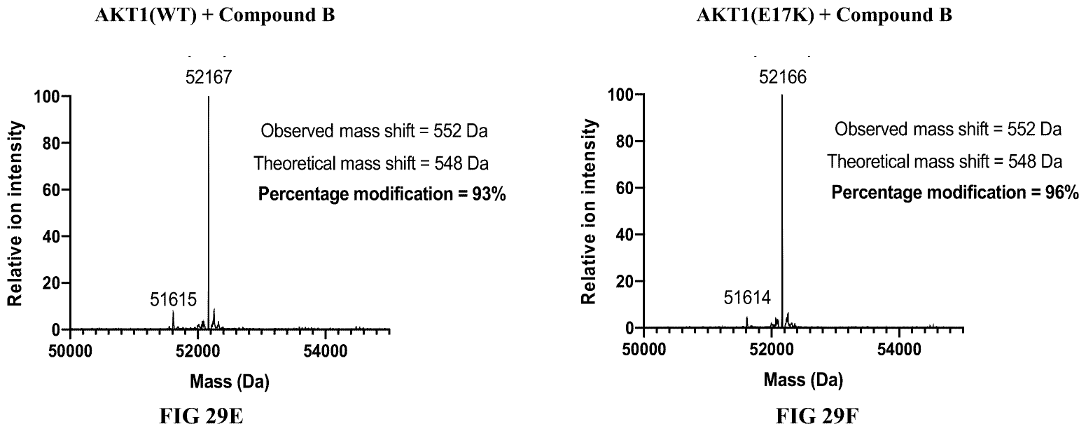

Covalent labelling selectivity were measured by intact-protein mass spectrometry. AKT1(WT) or AKT1(E17K) (1 μM) was incubated with inhibitor (5 μM) for 15 minutes. The covalent imine adducts were reduced with 5 mM NaBH₄ and the percentage labelling determined by LCMS. FIG. 29A-29F, provide tandem mass spectra for various experimental conditions. FIG. 29A-29B illustrate tandem mass spectra for AKT1 wild-type and AKT1 E17K, respectively. FIG. 29C illustrates tandem mass spectra for AKT1 wild-type and Compound A. FIG. 29D illustrates tandem mass spectra for AKT1 E17K and Compound A. FIG. 29E illustrates tandem mass spectra for AKT1 wild-type and Compound B. FIG. 29F illustrates tandem mass spectra for AKT1 E17K and Compound B.

### Example 148: Covalent Labeling Study of AKT1 E17K and Compound A

Identification of the sites of covalent modification by tandem mass spectrometry: AKT1(E17K) (5 μM) was incubated with excess compound (Compound A) (50 μM) for 15 minutes and then treated with NaBH₄ (5 mM) to capture the reversible imine through reductive amination. The labelled protein was digested with trypsin and the peptides analyzed by tandem mass spectrometry. MS/MS spectra were searched using a database comprised of the peptide sequence for the recombinant AKT1, along with the reverse sequence as decoy. In addition to typical variable modifications, a variable modification on lysine corresponding to compound addition (minus H2O) was allowed (e.g. 564.605 Da: 18 Da=546.605 Da). Peptides bearing lysine residues modified with Compound A were identified by spectral matching. Parent Ion: [GK(Compound A)YIKTWRPR]⁺⁵ m/z=371.3965 (z=5); −0.69 ppm.

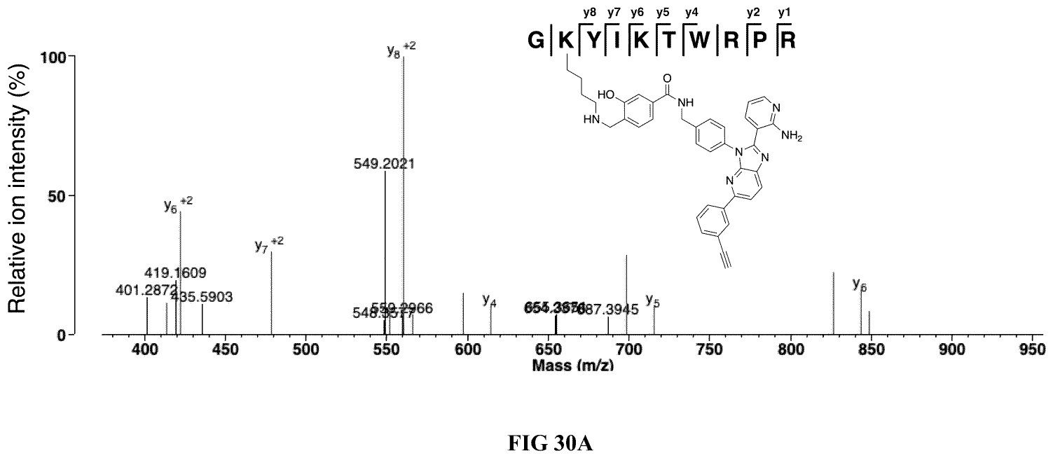

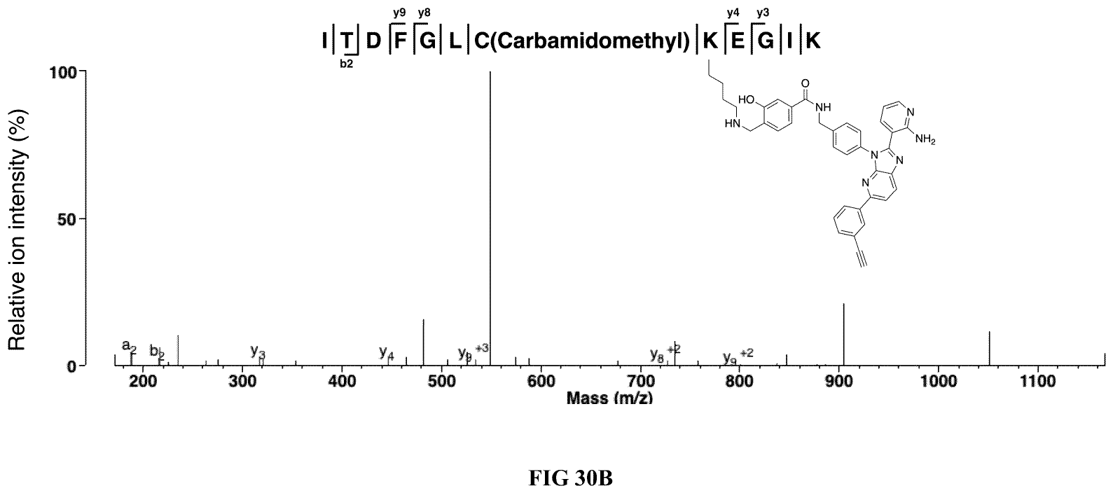

Table 5 provides the calculated ion fragments for the covalent modification of K17 in AKT1(E17K) by Compound A. FIG. 30A illustrates_{MS/MS} spectra of AKT1(E17K) and Compound A which demonstrate K17 modification. Observed fragment ions m/z (y): 843.4948, 715.3998, 614.3521, 272.1717, and 175.1190. Observed fragment ions m/z (y⁺²): 560.3247, 478.7931, 422.2510, and 358.2035. The observed fragment at m/z=549.2021, corresponds to

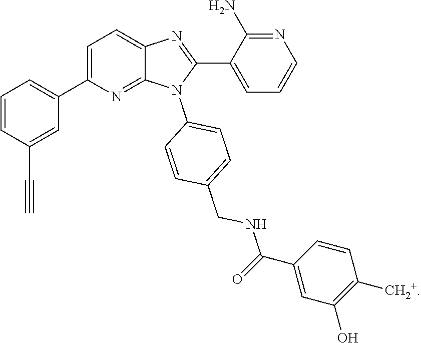

Table 6 provides the calculated ion fragments for the covalent modification of K297 in AKT1(E17K) by Compound A. FIG. 30A illustrates_{MS/MS} spectra of AKT1(E17K) and Compound A which demonstrate K297 modification. Observed fragment ions m/z (b): 215.139. Observed fragment ions m/z (y): 446.2609, and 317.2183. Observed fragment ions m/z (y⁺²): 800.3819 and 726.8477. Observed fragment ions m/z (y⁺³): 533.9238. The observed fragment at m/z=549.2021, corresponds to

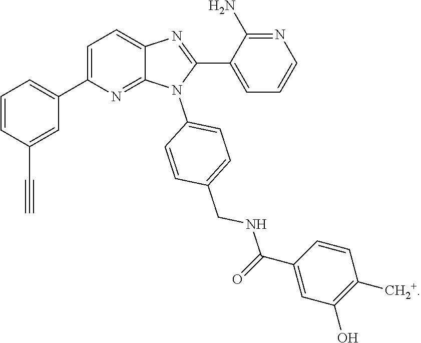

TABLE 5
Calculated m/z ion fragments for AKT1
E17K and Compound A: K17 modification.

| b, m/z | b⁺², m/z | bₓ | | yₓ | y, m/z | y⁺², m/z |
|---|---|---|---|---|---|---|
| — | — | 1 | G | 10 | — | — |
| 734.3198 | 367.6635 | 2 | K(Compound | 9 | 1795.9332 | 898.4702 |
| | | | A) | | | |
| 897.3831 | 449.1952 | 3 | Y | 8 | 1119.6422 | 560.3247 |
| 1010.4672 | 505.7372 | 4 | I | 7 | 956.5788 | 478.7931 |
| 1138.5621 | 569.7847 | 5 | K | 6 | 843.4948 | 422.2510 |
| 1239.6098 | 620.3085 | 6 | T | 5 | 715.3998 | 358.2035 |
| 1425.6891 | 713.3482 | 7 | W | 4 | 614.3521 | 307.6797 |
| 1581.7902 | 791.3988 | 8 | R | 3 | 428.2728 | 214.6401 |
| 1678.8430 | 839.9251 | 9 | P | 2 | 272.1717 | 136.5895 |
| — | — | 10 | R | 1 | 175.1190 | 88.0631 |

TABLE 6
Calculated m/z ion fragments for AKT1
E17K and Compound A: K297 modification.

| b, m/z | b⁺², m/z | bₓ | | yₓ | y, m/z | y⁺², m/z | y⁺³, m/z |
|---|---|---|---|---|---|---|---|
| — | — | 1 | I | 12 | — | — | 643.6433 |
| 215.139 | — | 2 | T | 11 | 1815.8312 | 908.4192 | 605.9486 |
| 330.166 | — | 3 | D | 10 | 1714.7835 | 857.8954 | 572.2661 |
| 477.2344 | — | 4 | F | 9 | 1599.7566 | 800.3819 | 533.9238 |
| 534.2558 | — | 5 | G | 8 | 1452.6881 | 726.8477 | 484.901 |
| 647.3399 | — | 6 | L | 7 | 1395.6667 | 698.337 | 465.8938 |
| 807.3706 | — | 7 | C(Carbamido- | 6 | 1282.5826 | 641.7949 | 42.1991 |
| | | | methyl) | | | | |
| 1483.662 | 742.3344 | 8 | K(Compound | 5 | 1122.552 | 561.7796 | 374.8556 |
| | | | A) | | | | |
| 1612.704 | 808.8557 | 9 | E | 4 | 446.2609 | 223.6341 | 149.4252 |
| 1669.726 | 835.3665 | 10 | G | 3 | 317.2183 | 159.1128 | 106.411 |
| 1782.81 | 891.9085 | 11 | I | 2 | 260.1969 | 130.6021 | 87.4039 |
| — | — | 12 | K | 1 | 147.1128 | 74.06 | 49.7092 |

As seen in FIG. 30A, a peptide comprising K(17 and proximate amino acid residues was identified via tandem mass spectrometry. This peptide is covalently bound to Compound A via the side chain nitrogen of 1K17 in the manner shown in FIG. 30A. The covalent adduct shown in FIG. 2A was formed by sodium borohydride reduction of the imine connecting K(17 to Compound A. These results demonstrate that Compound A forms a reversible covalent bond to the side chain of 1K17 via an imine functional group.

As seen in FIG. 301B, a peptide comprising 1(297 and proximate amino acid residues was identified via tandem mass spectrometry. This peptide is covalently bound to Compound A via the side chain nitrogen of 1(297 in the manner shown in FIG. 30B. The covalent adduct shown in FIG. 30B was formed by sodium borohydride reduction of the imine connecting 1(297 to Compound A. These results demonstrate that Compound A forms a reversible covalent bond to the side chain of 1(297 via an imine functional group.

### Example 149: Washout Treatment with Compound B

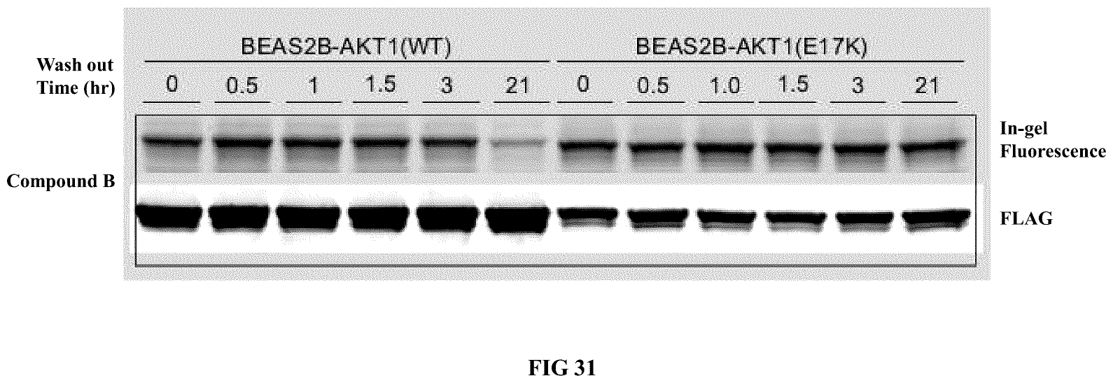

FIG. 31 shows the results of the washout treatment following incubation of Compound B with wild-type AKT1 (WT-AKT1) (left 6 lanes) and E17K AKT1 (right 6 lanes). As shown by the in-gel fluorescence levels in FIG. 31 (left), following 21 hours of dilution, the extent of Compound B binding to WT-AKT1 is markedly diminished compared to the extent of Compound B binding to WT-AKT1 following, e.g., 1 hour of dilution. These results indicate that a significant amount of Compound B binding to WT-AKT1 is either not covalent in nature or is reversible at a relatively rapid rate. However, even after 21 hours of dilution, a detectable amount of Compound B remains bound to WT-AKT1, indicating some degree of covalent binding between Compound B and WT-AKT1. In contrast, FIG. 31 (right) shows that the extent of Compound B binding to E17K AKT-1 after 21 hour of washout treatment is roughly equivalent to binding after 1 hour of washout. These results indicate a substantial extent of covalent adduct formation between Compound B and E17K AKT1. Further, the difference between Compound B binding to WT-AKT1 and E17K AKT1 demonstrates that Compound B binds with substantial selectively to K17 in E17K AKT1.

### Example 150: AKT1 Inhibition Data for Compound A and Compound B

Determination of compound dissociation rate by intact protein mass spectrometry:Recombinant AKT1 containing appropriate mutations (1 μM) was treated with inhibitor (5 μM) at room temperature. After 15 minutes, ARQ-092 (50 μM final concentration) was added to prevent aldehyde-containing compound rebinding after initial dissociation. At various timepoints, aliquots were removed and quenched with 5 mM NaBH₄ to capture the reversible imine through reductive amination, then analyzed by LCMS. Deconvoluted monoisotopic intensities of unmodified vs. compound-modified mass peaks were compared.

Identification of site of covalent modification by tandem mass spectrometry: AKT1(WT) or AKT1(E17K) (5 μM) was incubated with excess compound (Compound A or Compound B) (50 μM) for 15 minutes and then treated with NaBH₄ (5 mM) to capture the reversible imine through reductive amination. The labelled protein was digested with trypsin and the peptides analyzed by tandem mass spectrometry. MS/MS spectra were searched using a database comprised of the peptide sequence for the recombinant AKT1, along with the reverse sequence as decoy. In addition to typical variable modifications, a variable modification on lysine corresponding to compound addition (minus H2O) was allowed (e.g. 564.605 Da: 18 Da=546.605 Da).

Measurement of target engagement durability in intact cells: Beas-2b cells stably over-expressing either Flag-AKT1 WT or Flag-AKT1 E17K were treated with compound for 30 minutes at 37 C. The media was then aspirated and replaced with fresh media and the cells were incubated at 37° C. At various timepoints, cells were washed with PBS and then lysed in buffer containing 5 mM NaBH₄. After 1 h, the lysate was precipitated in MeOH/CHCl₃, resuspended in 1% SDS in PBS and then subjected to copper catalyzed click chemistry with TAMRA-biotin-azide. The samples were precipitated again and then resuspended in 1.5× SDS loading buffer, separated by SDS-PAGE and analyzed by in-gel fluorescence and western blot.

Measurement of phospho-AKT inhibition and treatment durability in intact cells: Cells were treated for 2 hrs with compound, then washed twice with fresh media with a 5 min 37 C incubation between washes. After additional incubation for 0, 8 or 24 h at 37 C, cells were washed with PBS and lysed in buffer containing protease and phosphatase inhibitors. Lysates were analyzed by SDS-PAGE/western blot using standard methods.

Determining live cell AKT1 E17K, AKT1 WT, and AKT2 target engagement by NanoBRET competitive probe displacement: Live cell target engagement assays were performed as described (Vasta, J. D. et al. Cell Chem Biol 2018, 25(11), 206-214). Briefly, HEK293 cells were transfected with plasmids encoding kinase-NanoLuciferase fusion proteins overnight. Cells were then treated with serial dilutions of compound and an ˜EC50 concentration of fluorescently-tagged ATP-competitive tracer (Promega). After incubation for 2 h at 37 C, luciferase substrate and extracellular luciferase inhibitor were added to all wells, and luminescent intensity at 460 nm and 600 nm were measured on a multimode plate-reader. The ratio of E₆₀₀/E₄₆₀ was calculated to give the tracer engagement signal (BRET).

Table 7 provides IC₅₀ values for Compound A, Compound B, and Miransertib (ARQ-092). Table 7 includes IC₅₀ values for the selected compounds; with compounds having a IC₅₀ of less than or equal to 100 nM as A; 100 nM>B≥500 nM as B; and greater than 500 nM as C.

TABLE 7
IC₅₀ values for Compound A, Compound B, and Miransertib

| | AKT1 E17K | AKT1 WT | AKT2 |
|---|---|---|---|
| | IC₅₀ | IC₅₀ | IC₅₀ |
| Compound | (nM) | (nM) | (nM) |
| Miransertib (ARQ-092) | A | A | A |
| 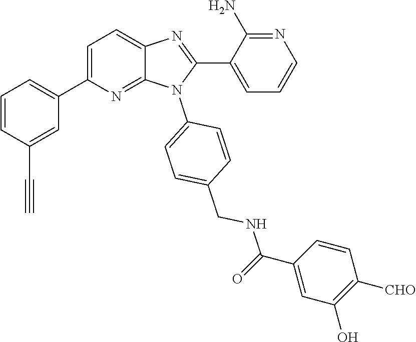 [SMILES: C#Cc1cccc(-c2ccc3nc(-c4cccnc4N)n(-c4ccc(CNC(=O)c5ccc(C=O)c(O)c5)cc4)c3n2)c1] | B | A | C |
| Compound A | | | |
| 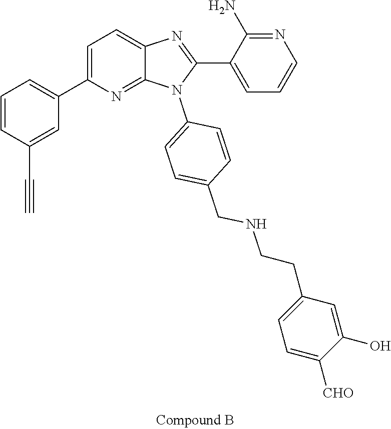 [SMILES: C#Cc1cccc(-c2ccc3nc(-c4cccnc4N)n(-c4ccc(CNCCc5ccc(C=O)c(O)c5)cc4)c3n2)c1] | A | B | C |

### Example 151: Dissociation Rate Data for Compound a and Compound B in Wild-Type AKT1 and AKT1 E17K

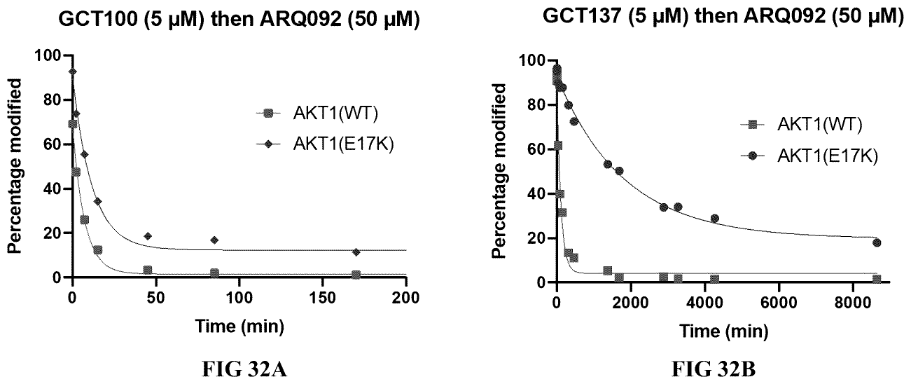

AKT1(E17K or WT) (1 μM) was incubated with Compound A or Compound B (5 μM) for 15 minutes in 25 mM HEPES pH 8.0, 150 mM NaCl buffer. Then ARQ-092 (50 μM) was added and aliquots quenched with 5 mM NaBH₄ at a series of timepoints and the percentage labelling determined by LCMS. FIG. 32A and FIG. 32B provide competition data (dissociation rates) for Compound A and Compound B in both wild-type AKT1 and AKT1 E17K against ARQ-092. Table 8 provides half-life data based on the dissociation studies described herein.

TABLE 8
Half-life data for Compound A and Compound B

| | AKT1 (WT) | AKT1 E17K | Off-rate Selectivity |
|---|---|---|---|
| | t_{1/2} (mins) | t_{1/2} (mins) | E17K/WT |
| Compound A | 5 | 8.2 | 1.6 |
| Compound B | 72.7 | 1037 | 14.7 |

1.-89. (canceled)

90. An AKT1 protein covalently bound to a compound, wherein the compound is covalently bound to a lysine residue of the AKT1 protein.

91. The AKT1 protein of claim 90, wherein the AKT1 protein comprises a E17K mutation, a E40K mutation, or a E49K mutation.

92. The AKT1 protein of claim 90, wherein the lysine residue is selected from K17, K40, K49, K158, K163, K179, K267, and K297.

93. An in vivo engineered AKT1 protein comprising a non-naturally occurring reversible covalent modification at a lysine residue, the reversible covalent modification being generated from an in vivo nucleophilic reaction between an exogenous aromatic aldehyde and a lysine residue of AKT1, wherein the exogenous aromatic aldehyde undergoes a nucleophilic addition with the amine functional group on the lysine residue and forming a carbon-nitrogen double bond between the exogenous aromatic aldehyde and the amine functional group on the lysine residue.

94. The in vivo engineered AKT1 protein of claim 93, wherein the AKT1 protein comprises a E17K mutation.

95. The in vivo engineered AKT1 protein of claim 93, wherein the lysine residue is selected from K17, K40, K49, K158, K163, K179, K267, and K297.

96. A compound of Formula (II): 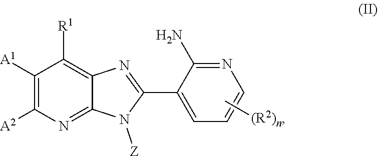
or a pharmaceutically acceptable salt thereof; wherein,
R¹ and R² are each independently selected from hydrogen, halogen, C₁₋₄ alkyl, C₁₋₄ haloalkyl, —OR¹⁰, —SR¹⁰, —N(R¹⁰)₂, —NO₂, and —CN;
n is selected from 0, 1, 2, and 3;
A¹ and A² are each independently selected from:
hydrogen, halogen, —OR¹¹, —SR¹¹, —N(R¹¹)₂, —C(O)R¹¹, —C(O)N(R¹¹)₂, —N(R¹¹)C(O)R¹¹, —NO₂, and —CN;
C₁₋₆ alkyl, C₂₋₆ alkenyl, and C₂₋₆ alkynyl, any of which is optionally substituted with one or more substituents independently selected from halogen, —OR¹¹, —SR¹¹, —N(R¹¹)₂, —C(O)N(R¹¹)₂, —N(R¹¹)C(O)R¹¹, ═O, ═S, ═N(R¹¹), and —CN; and
3- to 10-membered heterocycle and C₃₋₁₀ carbocycle, any one of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —S(O)₂N(R¹¹)₂, —NO₂, ═O, ═S, ═N(R¹¹), and —CN;
C₁₋₆ alkyl, C₂₋₆ alkenyl, and C₃₋₆ alkynyl, any one of which is optionally substituted with one or more substituents independently selected from:
halogen, —OR¹¹, —SR¹¹, —N(R¹¹)₂, —C(O)R¹¹, —C(O)N(R¹¹)₂, —N(R¹¹)C(O)R¹¹, —N(R¹¹)S(O)₂R¹¹, —C(O)OR¹¹, —OC(O)R¹¹, —S(O)R¹¹, —S(O)₂R¹¹, —NO₂, ═O, ═S, ═N(R¹¹), and —CN; and
C₃₋₁₀ carbocycle and 3- to 10-membered heterocycle; any one of which is optionally substituted with one or more substituents selected from halogen, C₁₋₄ alkyl, C₁₋₄ haloalkyl, —OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O;
Z is represented by 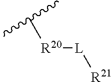
wherein,
R²⁰ is selected from 5- to 6-membered heterocyclene and phenylene, any of which is optionally substituted with one or more substituents independently selected from halogen, C₁₋₄ alkyl, C₁₋₄ haloalkyl —OR¹², —SR¹², —N(R¹²)₂, —NO₂, ═O, ═S, ═N(R¹²), and —CN;
L is a bond or represented by -L¹-L²-L³-L⁴-, wherein each L¹, L², L³, and L⁴ is independently selected from (a) and (b):
(a) —O—, —S—, —S(O)—, —S(O)₂—, —N(R¹⁴)—, —N(R¹⁴)C(O)—, —N(R¹⁴)C(O)O—, —N(R¹⁴)S(O)₂—, —N(R¹⁴)S(O)₂N(R¹⁴)—, —N(R¹⁴)N(R¹⁴)—, (R¹⁴)NC(O)N(R¹⁴), and (R¹⁴)NC(O)N(R¹⁴)N(R¹⁴); and
(b) C₁₋₆ alkylene, C₂₋₆ alkenylene, C₂₋₆ alkynylene, C₃₋₈ carbocyclene, and 3- to 8-membered heterocyclene, any one of which is optionally substituted with one or more substituents independently selected from halogen, —OR¹³, —SR¹³, —N(R¹³)₂, ═O, ═S, and —CN;
wherein L², L³, and L⁴ are each optionally absent;
wherein no more than two of L¹, L², L³, and L⁴ are selected from (a) and the two selected are not adjacent to each other;
R²¹ is selected from 5- to 6-membered heterocycle and C₃₋₆ carbocycle, any of which is substituted with —C(O)H, wherein R²¹ is further optionally substituted with one or more substituents independently selected from:
halogen, C₁₋₄ alkyl, C₁₋₄ haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, ═O, ═S, ═N(R¹⁵), and —CN; and
R¹⁰, R¹¹, R¹², R¹³, R¹⁴, and R¹⁵ are each independently selected at each occurrence from hydrogen, C₁₋₄ alkyl, C₁₋₄ haloalkyl, C₃₋₈ carbocycle, and 3- to 8-membered heterocycle, wherein the C₃₋₈ carbocycle and 3- to 8-membered heterocycle are each optionally substituted with one or more substituents independently selected from halogen, C₁₋₄ alkyl, C₁₋₄ haloalkyl, and ═O.

97. The compound or salt of claim 96, wherein R¹ is selected from hydrogen, halogen, C₁₋₄ alkyl, and C₁₋₄ haloalkyl.

98. The compound or salt of claim 96, wherein n is selected from 0 and 1.

99. The compound or salt of claim 96, wherein A¹ is selected from hydrogen, halogen, optionally substituted C₃₋₁₀ carbocycle and optionally substituted 3- to 10-membered heterocycle.

100. The compound or salt of claim 96, wherein A² is selected from:
hydrogen, fluoro, chloro, bromo, —OR¹¹, —N(R¹¹)₂;
C₁₋₄ alkyl and C₂₋₄ alkynyl, any of which is optionally substituted with one or more substituents independently selected from halogen and —OR¹¹; and
C₃₋₁₀ carbocycle and 3- to 10-membered heterocycle, any of which is optionally substituted with one or more substituents independently selected from:
halogen, —OR¹¹, —SR¹¹, —N(R¹)₂, —C(O)R¹¹, —C(O)N(R¹)₂, —N(R¹¹)C(O)R¹¹, —N(R¹¹)S(O)₂R¹¹, —C(O)OR¹, —OC(O)R¹¹, —S(O)R¹¹, —S(O)₂R¹¹, —NO₂, ═O, ═S, ═N(R¹¹), —CN, C₁₋₆ alkyl, and C₁₋₆ haloalkyl; and
C₃₋₁₀ carbocycle and 3- to 10-membered heterocycle; any of which is optionally substituted with one or more substituents selected from: halogen, OR¹¹, —N(R¹¹)₂, —C(O)R¹¹, —C(O)N(R¹¹)₂, —N(R¹¹)C(O)R¹¹, —C(O)OR¹¹, —OC(O)R¹¹, —NO₂, ═O, ═S, ═N(R¹¹), —CN, C₃₋₆ carbocycle, and 3- to 6-membered heterocycle, wherein the C₃₋₆ carbocycle and 3- to 6-membered heterocycle are each optionally substituted with one or more substituents independently selected from halogen, C₁₋₄ alkyl, and ═O; and
R¹¹ is independently selected from: hydrogen, C₁₋₃ alkyl, C₁₋₃ haloalkyl, and C₃₋₄ carbocycle.

101. The compound or salt of claim 100, wherein A² is selected from hydrogen, fluoro, chloro

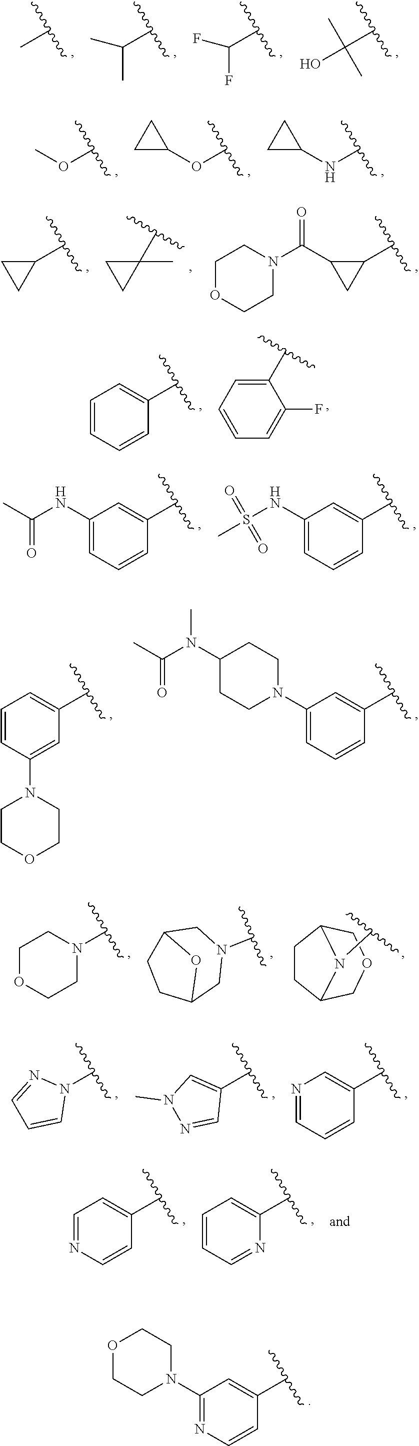

102. The compound or salt of claim 96, wherein R²⁰ is selected from 5- to 6-membered heterocyclene and phenylene, any of which is optionally substituted with one or more substituents independently selected from halogen, C₁₋₄ alkyl, C₁₋₄ haloalkyl, —OR¹², —N(R¹²)₂, —C(O)R¹², —C(O)N(R¹²)₂, —N(R¹²)C(O)R¹², ═O, and —CN.

103. The compound or salt of claim 96, wherein L is selected from:

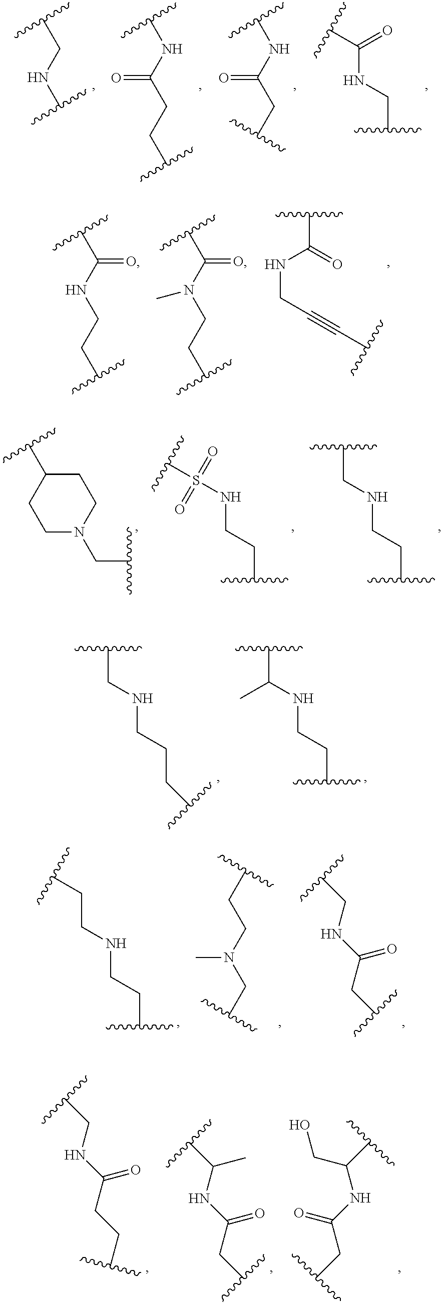

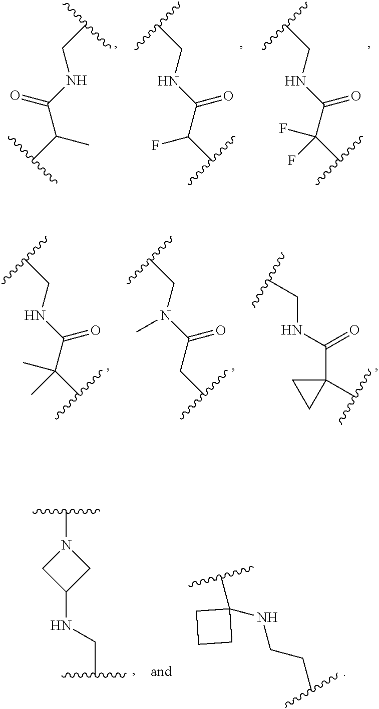

104. The compound or salt of claim 96, wherein R²¹ is selected from 5- to 6-membered heterocycle and C₅₋₆ carbocycle, any of which is substituted with —C(O)H, wherein R²¹ is further optionally substituted with one or more substituents independently selected from: halogen, C₁₋₄ alkyl, C₁₋₄ haloalkyl, —OR¹⁵, —SR¹⁵, —N(R¹⁵)₂, —B(OR¹⁵)₂, —C(O)R¹⁵, —C(O)N(R¹⁵)₂, —C(O)OR¹⁵, —OC(O)R¹⁵, —N(R¹⁵)C(O)R¹⁵, —N(R¹⁵)S(O)₂R¹⁵, —N(R¹⁵)C(O)N(R¹⁵)₂, —S(O)R¹⁵, —S(O)₂R¹⁵, —NO₂, and —CN.

105. The compound or salt of claim 104, wherein R²¹ is selected from:

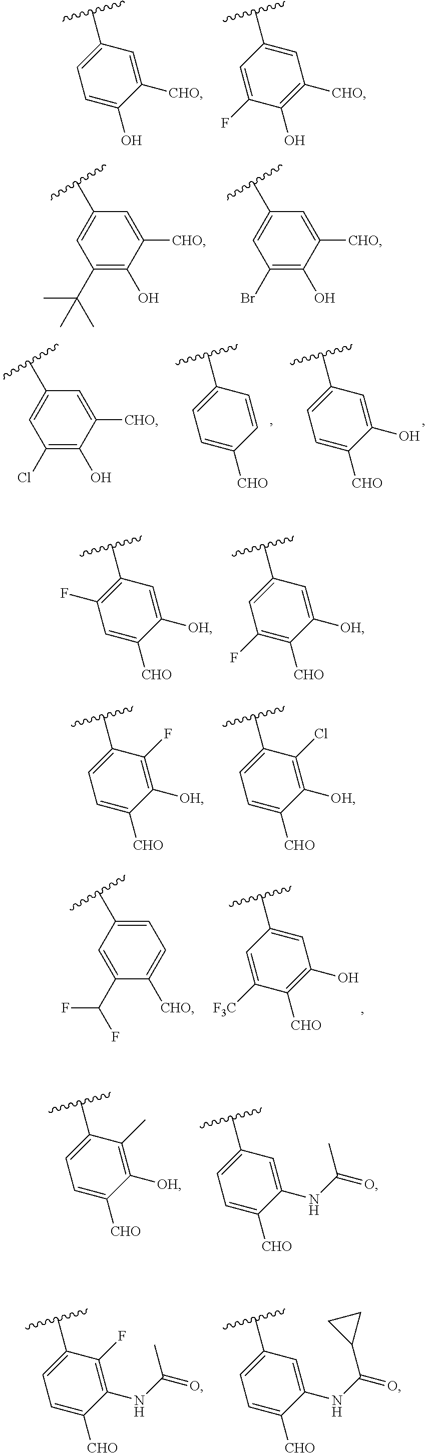

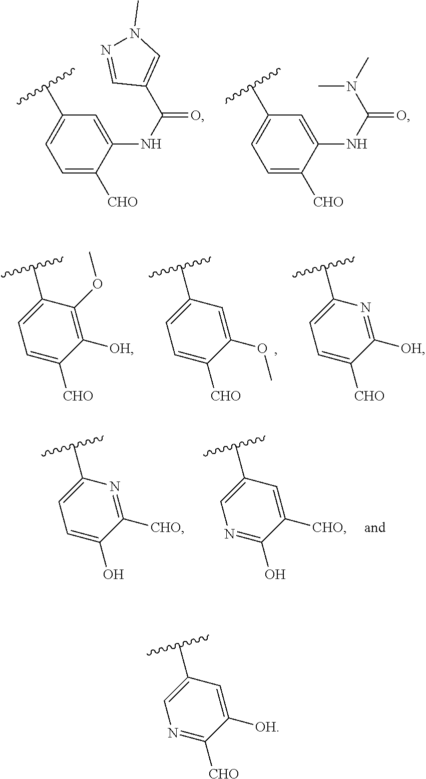

106. The compound of claim 96, wherein the compound of Formula (II) is a compound of Table 1, or a salt of any one thereof.

107. A pharmaceutical composition comprising a pharmaceutically acceptable excipient and a compound or salt of claim 96.

108. A method of selectively modulating activity a mutant AKT1 over a wild type AKT comprising administering to a subject in need thereof a compound or salt of claim 96, or a pharmaceutical composition of claim 107, wherein the wild type AKT is selected from wild type AKT1 and wild type AKT2.

109. A method of treating cancer in a subject in need thereof, the method comprising administering to the subject a compound or salt of claim 96, or a pharmaceutical composition of claim 107.


SEQUENCE LISTING (9 sequences)

SEQ ID NO: 1 - protein, 492 aa, Homo sapiens
HHHHHHENLYFQGSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASGTA

SEQ ID NO: 2 - protein, 492 aa, synthetic construct
HHHHHHENLYFQGSDVAIVKEGWLHKRGKYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASGTA

SEQ ID NO: 3 - protein, 6 aa, synthetic construct
HHHHHH

SEQ ID NO: 4 - protein, 456 aa, Homo sapiens
MSHHHHHHHHGSENLYFQSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDASAEHTDMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQM

SEQ ID NO: 5 - protein, 456 aa, synthetic construct
MSHHHHHHHHGSENLYFQSDVAIVKEGWLHKRGKYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDASAEHTDMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQM

SEQ ID NO: 6 - protein, 126 aa, synthetic construct
QVQLQESGGGLVQAGGSLRLSCAASGIDVRIKTMAWYRQAPGKQRELLASVLVSGSTNYADPVKGRFTISRDNAKNTVYLQMNKLIPDDTAVYYCNTYGRLRRDVWGPGTQVTVSSHHHHHHEPEA

SEQ ID NO: 7 - protein, 10 aa, synthetic construct
  SITE 2: note = N-(4-(2-(2-aminopyridin-3-yl)-5-(3-ethynylphenyl)-3 H-imidazo[4,5-b]pyridin-3-yl)benzyl)-4-formyl-3-hydroxyben zamide modified residue
GKYIKTWRPR

SEQ ID NO: 8 - protein, 5 aa, synthetic construct
VVYRD

SEQ ID NO: 9 - protein, 12 aa, synthetic construct
  SITE 7: note = Carbamidomethyl modified residue
  SITE 8: note = N-(4-(2-(2-aminopyridin-3-yl)-5-(3-ethynylphenyl)-3 H-imidazo[4,5-b]pyridin-3-yl)benzyl)-4-formyl-3-hydroxyben zamide modified residue
ITDFGLCKEGIK